(19) United States
(12) Patent Application Publication
(10) Pub. No.: US 2024/0292741 A1
(43) Pub. Date: Aug. 29, 2024

(54) HETEROCYCLIC COMPOUND AND ORGANIC LIGHT-EMITTING DEVICE USING SAME

(71) Applicant: LT MATERIALS CO., LTD., Yongin-si, Gyeonggi-do (KR)

(72) Inventors: Nam-Jin LEE, Yongin-si (KR); Na-Yeong KIM, Yongin-si (KR); Hyo-Kyun HAM, Yongin-si (KR); Won-Jang JEONG, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/564,217

(22) PCT Filed: Aug. 23, 2022
(86) PCT No.: PCT/KR2022/012593

§ 371 Date: Nov. 27, 2023

(30) Foreign Application Priority Data
Aug. 23, 2021 (KR) 10-2021-0111078

(51) Int. Cl. H10K 85/60; C07D 307/91; C07D 333/76; C07D 409/12; C07D 409/14; C09K 11/06; H10K 50/15; H10K 50/18

(52) U.S. Cl. CPC H10K 85/636; C07D 307/91; C07D 333/76; C07D 409/12; C07D 409/14; C09K 11/06; H10K 85/633; C09K 2211/1018; H10K 50/15; H10K 50/181; H10K 85/615; H10K 85/622; H10K 85/626; H10K 85/6574; H10K 85/6576

(57) ABSTRACT

The present application provides a heterocyclic compound which may significantly improve a service life, efficiency, electrochemical stability, and thermal stability of an organic light emitting device, and an organic light emitting device in which the heterocyclic compound is contained in an organic material layer.

### TECHNICAL FIELD

This application claims priority to and the benefit of Korean Patent Application No. 10-2021-0111078 filed in the Korean Intellectual Property Office on Aug. 23, 2021, the entire contents of which are incorporated herein by reference.

The present application relates to a heterocyclic compound and an organic light emitting device using the same.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multi layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENT

### Patent Document

U.S. Pat. No. 4,356,429

### DISCLOSURE

### Technical Problem

The present invention has been made in an effort to provide a heterocyclic compound and an organic light emitting device including the same.

### Technical Solution

An exemplary embodiment of the present application provides a heterocyclic compound represented by the following Chemical Formula 1.

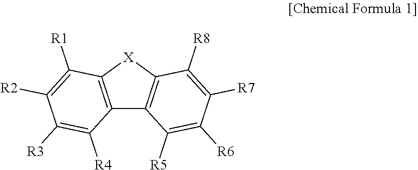

In Chemical Formula 1,
X is O; or S,
one of R1 to R8 is the following Chemical Formula A, one of the others is the following Chemical Formula B, and the others are the same as or different from each other, and are each independently selected from the group consisting of hydrogen; deuterium; a halogen group; a cyano group; a substituted or unsubstituted alkyl group having 1 to 60 carbon atoms; a substituted or unsubstituted aryl group having 6 to 60 carbon atoms; and a substituted or unsubstituted heteroaryl group having 2 to 60 carbon atoms,

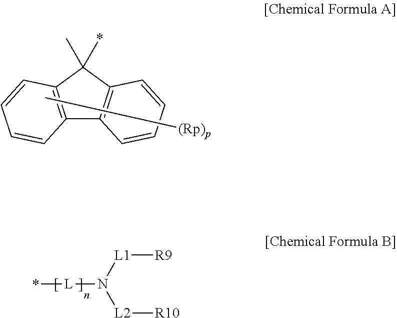

in Chemical Formulae A and B,
Rp is hydrogen; deuterium; a halogen group; a cyano group; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms, p is an integer from 0 to 8, and when p is 2 or higher, Rp's in the parenthesis are the same as or different from each other,
L, L1 and L2 are a direct bond; a substituted or unsubstituted arylene group having 6 to 60 carbon atoms; or a substituted or unsubstituted heteroarylene group having 2 to 60 carbon atoms,
R9 and R10 are the same as or different from each other, and are each independently a substituted or unsubstituted aryl group having 6 to 60 carbon atoms; or a substituted or unsubstituted heteroaryl group having 2 to 60 carbon atoms,
n is an integer from 0 to 3, and when n is 2 or higher, L's in the parenthesis are the same as or different from each other,
*is a position where Chemical Formula 1, Chemical Formula A and Chemical Formula B are each bonded, and
when R1 is Chemical Formula A, X is S.

Further, another exemplary embodiment of the present application provides an organic light emitting device including a first electrode, a second electrode and an organic material layer having one or more layers provided between the first electrode and the second electrode, in which one or more layers of the organic material layer include the heterocyclic compound represented by Chemical Formula 1.

### Advantageous Effects

A heterocyclic compound according to an exemplary embodiment of the present application can be used as a material for an organic material layer of an organic light emitting device. The heterocyclic compound can be used as a material for a hole injection layer, a hole transport layer, a light emitting layer, an electron transport layer, an electron injection layer, a charge generation layer, and the like in an organic light emitting device. In particular, the heterocyclic compound represented by Chemical Formula 1 can be used as a material for a hole transport layer or electron blocking layer of an organic light emitting device. In addition, when the heterocyclic compound represented by Chemical Formula 1 is used for an organic light emitting device, the driving voltage of the device can be lowered, the light efficiency of the device can be improved, and the service life characteristics of the device can be improved due to the thermal stability of the compound.

### DESCRIPTION OF DRAWINGS

FIGS. 1 to 3 each are views schematically illustrating a stacking structure of an organic light emitting device according to an exemplary embodiment of the present application.

### MODE FOR INVENTION

Hereinafter, the present application will be described in detail.

An exemplary embodiment of the present application provides a heterocyclic compound represented by the following Chemical Formula 1.

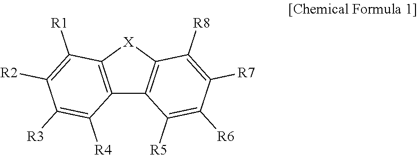

In Chemical Formula 1,
X is O; or S,
one of R1 to R8 is the following Chemical Formula A, one of the others is the following Chemical Formula B, and the others are the same as or different from each other, and are each independently selected from the group consisting of hydrogen; deuterium; a halogen group; a cyano group; a substituted or unsubstituted alkyl group having 1 to 60 carbon atoms; a substituted or unsubstituted aryl group having 6 to 60 carbon atoms; and a substituted or unsubstituted heteroaryl group having 2 to 60 carbon atoms,

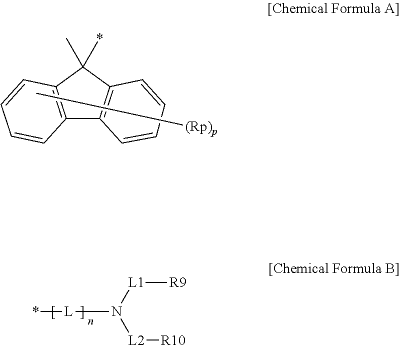

in Chemical Formulae A and B,
Rp is hydrogen; deuterium; a halogen group; a cyano group; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms, p is an integer from 0 to 8, and when p is 2 or higher, Rp's in the parenthesis are the same as or different from each other,
L, L1 and L2 are the same as or different from each other, and are each independently a direct bond; a substituted or unsubstituted arylene group having 6 to 60 carbon atoms; or a substituted or unsubstituted heteroarylene group having 2 to 60 carbon atoms,
R9 and R10 are the same as or different from each other, and are each independently a substituted or unsubstituted aryl group having 6 to 60 carbon atoms; or a substituted or unsubstituted heteroaryl group having 2 to 60 carbon atoms,
n is an integer from 0 to 3, and when n is 2 or higher, L's in the parenthesis are the same as or different from each other,
*is a position where Chemical Formula 1, Chemical Formula A and Chemical Formula B are each bonded, and
when R1 is Chemical Formula A, X is S.

Since the compound represented by Chemical Formula 1 has excellent hole transport ability by having an amine group having hole characteristics as a substituent in a dibenzofuran group or dibenzothiophene group, and suppresses the pi-pi stacking of an aromatic ring and has a high LUMO level and a wide band gap and stability by having a specific substituent including a fluorene group, there is an effect of lowering the driving voltage of an organic light emitting device and exhibiting excellent efficiency and service life characteristics when the compound represented by Chemical Formula 1 is used in the organic light emitting device.

In the present specification, the term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more substituents are substituted, the two or more substituents may be the same as or different from each other.

In the present specification, “substituted or unsubstituted” means being unsubstituted or substituted with one or more substituents selected from the group consisting of deuterium; a halogen group; a cyano group; a straight-chained or branched alkyl having 1 to 60 carbon atoms; a straight-chained or branched alkenyl having 2 to 60 carbon atoms; a straight-chained or branched alkynyl having 2 to 60 carbon atoms; a monocyclic or polycyclic cycloalkyl having 3 to 60 carbon atoms; a monocyclic or polycyclic heterocycloalkyl having 2 to 60 carbon atoms; a monocyclic or polycyclic aryl having 6 to 60 carbon atoms; a monocyclic or polycyclic heteroaryl having 2 to 60 carbon atoms; —SiRR′R″; —P(═O)RR′; an alkylamine having 1 to 20 carbon atoms; a monocyclic or polycyclic arylamine having 6 to 60 carbon atoms; and a monocyclic or polycyclic heteroarylamine having 2 to 60 carbon atoms, or being unsubstituted or substituted with a substituent to which two or more substituents selected from the above exemplified substituents are linked, and means that R, R′ and R″ are the same as or different from each other, and are each independently hydrogen; deuterium; a halogen; a substituted or unsubstituted alkyl having 1 to 60 carbon atoms; a substituted or unsubstituted aryl having 6 to 60 carbon atoms; or a substituted or unsubstituted heteroaryl having 2 to 60 carbon atoms.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen; or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or 2H.

In an exemplary embodiment of the present application, the isotope means an atom with the same atomic number (Z), but different mass numbers (A), and the isotope may be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, the deuterium content of 20% in a phenyl group represented by

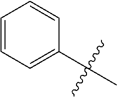 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

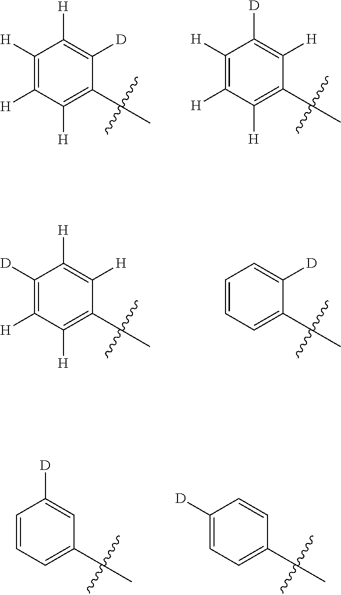 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, the alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, the alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group, and the like, but are not limited thereto.

In the present specification, the alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, an alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, the cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group includes 0, S, Se, N or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, the aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, a phosphine oxide group is represented by —P(═O)R101R102, and R101 and R102 are the same as or different from each other, and may be each independently a substituent composed of at least one of hydrogen; deuterium; a halogen group; an alkyl group; an alkenyl group; an alkoxy group; a cycloalkyl group; an aryl group; and a heterocyclic group. Specific examples of the phosphine oxide group include a diphenylphosphine oxide group, dinaphthylphosphine oxide, and the like, but are not limited thereto.

In the present specification, a silyl group includes Si and is a substituent to which the Si atom is directly linked as a radical, and is represented by —SiR104R105R106, and R104 to R106 are the same as or different from each other, and may be each independently a substituent composed of at least one of hydrogen; deuterium; a halogen group; an alkyl group; an alkenyl group; an alkoxy group; a cycloalkyl group; an aryl group; and a heterocyclic group. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, the fluorenyl group may be substituted, and adjacent substituents may be bonded to each other to form a ring.

In the present specification, the spiro group is a group including a spiro structure, and may have 15 to 60 carbon atoms. For example, the spiro group may include a structure in which a 2,3-dihydro-1H-indene group or a cyclohexane group is spiro-bonded to a fluorenyl group. Specifically, the spiro group may include any one of the groups of the following structural formulae.

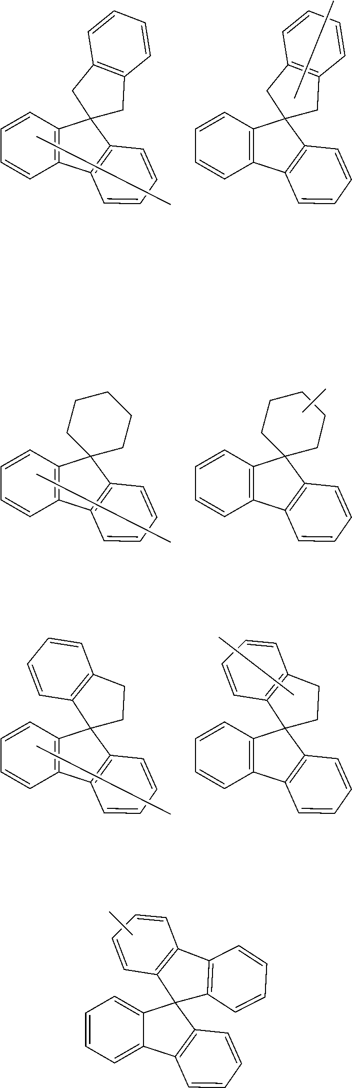

In the present specification, the heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b, f]azepin group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrodibenzo[b,e][1, 4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, an arylene group means that there are two bonding positions in an aryl group, that is, a divalent group. The above-described description on the aryl group may be applied to the arylene group, except that the arylene groups are each a divalent group. Further, a heteroarylene group means that there are two bonding positions in a heteroaryl group, that is, a divalent group. The above-described description on the heteroaryl group may be applied to the arylene group, except that heteroarylene groups are each a divalent group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted with the same carbon in an aliphatic ring may be interpreted as groups which are “adjacent” to each other.

A heterocyclic compound according to an exemplary embodiment of the present application is represented by Chemical Formula 1. More specifically, the heterocyclic compound represented by Chemical Formula 1 may be used as a material for an organic material layer of an organic light emitting device by the structural characteristics of the core structure and the substituent as described above.

In an exemplary embodiment of the present application, the deuterium content of the compound represented by Chemical Formula 1 may be 0% or more and 100% or less.

In an exemplary embodiment of the present application, the deuterium content of the compound represented by Chemical Formula 1 may be 10% or more and 100% or less.

In an exemplary embodiment of the present application, the deuterium content of the compound represented by Chemical Formula 1 may be 20% or more and 100% or less.

In an exemplary embodiment of the present application, the deuterium content of the compound represented by Chemical Formula 1 may be 40% or more and 100% or less.

In an exemplary embodiment of the present application, one of R1 to R8 of Chemical Formula 1 is Chemical Formula A, one of the others is Chemical Formula B, and the other are the same as or different from each other, and may be each independently selected from the group consisting of hydrogen; deuterium; a halogen group; a cyano group; a substituted or unsubstituted alkyl group having 1 to 60 carbon atoms; a substituted or unsubstituted aryl group having 6 to 60 carbon atoms; and a substituted or unsubstituted heteroaryl group having 2 to 60 carbon atoms.

In an exemplary embodiment of the present application, one of R1 to R8 is Chemical Formula A, one of the others is Chemical Formula B, and the others are the same as or different from each other, and may be each independently selected from the group consisting of hydrogen; deuterium; a halogen group; a cyano group; a substituted or unsubstituted alkyl group having 1 to 40 carbon atoms; a substituted or unsubstituted aryl group having 6 to 40 carbon atoms; and a substituted or unsubstituted heteroaryl group having 2 to 40 carbon atoms.

In an exemplary embodiment of the present application, one of R1 to R8 is the following Chemical Formula A, one of the others is Chemical Formula B, and the others are the same as or different from each other, and may be each independently selected from the group consisting of hydrogen; deuterium; a halogen group; a cyano group; a substituted or unsubstituted alkyl group having 1 to 20 carbon atoms; a substituted or unsubstituted aryl group having 6 to 20 carbon atoms; and a substituted or unsubstituted heteroaryl group having 2 to 20 carbon atoms.

In an exemplary embodiment of the present application, one of R1 to R8 is Chemical Formula A, one of the others is Chemical Formula B, and the others are the same as or different from each other, and may be each independently hydrogen; or deuterium.

In an exemplary embodiment of the present application, one of R1 to R8 is Chemical Formula A, one of the others is Chemical Formula B, and the other are hydrogen; or deuterium.

In an exemplary embodiment of the present application, one of R1 to R8 is Chemical Formula A, one of the others is Chemical Formula B, and the other are hydrogen.

In an exemplary embodiment of the present application, one of R1 to R8 is Chemical Formula A, one of the others is Chemical Formula B, and the others are deuterium.

In an exemplary embodiment of the present application, Rp of Chemical Formula A may be hydrogen; deuterium; a halogen group; a cyano group; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms.

In an exemplary embodiment of the present application, Rp may be hydrogen; deuterium; a halogen group; a cyano group; a substituted or unsubstituted alkyl group having 1 to 40 carbon atoms; a substituted or unsubstituted aryl group having 6 to 40 carbon atoms; or a substituted or unsubstituted heteroaryl group having 2 to 40 carbon atoms.

In an exemplary embodiment of the present application, Rp may be hydrogen; deuterium; a halogen group; a cyano group; a substituted or unsubstituted alkyl group having 1 to 20 carbon atoms; a substituted or unsubstituted aryl group having 6 to 20 carbon atoms; or a substituted or unsubstituted heteroaryl group having 2 to 20 carbon atoms.

In an exemplary embodiment of the present application, Rp is hydrogen; or deuterium.

In an exemplary embodiment of the present application, Rp is hydrogen.

In an exemplary embodiment of the present application, Rp is deuterium.

In an exemplary embodiment of the present application, p of Chemical Formula A is an integer from 0 to 8, and when p is 2 or higher, Rp's in the parenthesis are the same as or different from each other.

In an exemplary embodiment of the present application, p is 0.

In an exemplary embodiment of the present application, p is 1.

In an exemplary embodiment of the present application, p is 2.

In an exemplary embodiment of the present application, p is 3.

In an exemplary embodiment of the present application, p is 4.

In an exemplary embodiment of the present application, p is 5.

In an exemplary embodiment of the present application, p is 6.

In an exemplary embodiment of the present application, p is 7.

In an exemplary embodiment of the present application, p is 8.

In an exemplary embodiment of the present application, when p is 2 or higher, Rp's in the parenthesis are the same as or different from each other.

In an exemplary embodiment of the present application, X of Chemical Formula 1 is O; or S.

In an exemplary embodiment of the present application, X is O.

In an exemplary embodiment of the present application, X is S.

In an exemplary embodiment of the present application, when R1 of Chemical Formula 1 is Chemical Formula A, X is S. When X of Chemical Formula 1 is S, the compound has a higher glass transition temperature (Tg) than when X is O. Accordingly, when a thin film is manufactured using the compound, there is an advantage in that thermal stability is excellent.

In an exemplary embodiment of the present application, L, L1 and L2 of Chemical Formula B are the same as or different from each other, and may be each independently a direct bond; a substituted or unsubstituted arylene group having 6 to 60 carbon atoms; or a substituted or unsubstituted heteroarylene group having 2 to 60 carbon atoms.

In an exemplary embodiment of the present application, L, L1 and L2 are the same as or different from each other, and may be each independently a direct bond; a substituted or unsubstituted arylene group having 6 to 40 carbon atoms; or a substituted or unsubstituted heteroarylene group having 2 to 40 carbon atoms.

In an exemplary embodiment of the present application, L, L1 and L2 are the same as or different from each other, and may be each independently a direct bond; a substituted or unsubstituted arylene group having 6 to 20 carbon atoms; or a substituted or unsubstituted heteroarylene group having 2 to 20 carbon atoms.

In an exemplary embodiment of the present application, L may be a direct bond; or a substituted or unsubstituted arylene group having 6 to 20 carbon atoms.

In an exemplary embodiment of the present application, L may be a direct bond; a substituted or unsubstituted phenylene group; or a substituted or unsubstituted biphenylene group.

In an exemplary embodiment of the present application, L may be a direct bond; a phenylene group unsubstituted or substituted with one or more deuteriums; or a biphenylene group unsubstituted or substituted with one or more deuteriums.

In an exemplary embodiment of the present application, n of Chemical Formula B is an integer from 0 to 3, and when n is 2 or higher, L's in the parenthesis may be the same as or different from each other.

In an exemplary embodiment of the present application, n is 0.

In an exemplary embodiment of the present application, n is 1.

In an exemplary embodiment of the present application, n is 2.

In an exemplary embodiment of the present application, n is 3.

In an exemplary embodiment of the present application, when n is 2 or higher, L's in the parenthesis are the same as or different from each other.

In an exemplary embodiment of the present application, L1 and L2 are the same as or different from each other, and may be each independently a direct bond; a substituted or unsubstituted phenylene group.

In an exemplary embodiment of the present application, L1 and L2 are the same as or different from each other, and may be each independently a direct bond; a phenylene group unsubstituted or substituted with one or more deuteriums.

In an exemplary embodiment of the present application, R9 and R10 of Chemical Formula B are the same as or different from each other, and may be each independently a substituted or unsubstituted aryl group having 6 to 60 carbon atoms; or a substituted or unsubstituted heteroaryl group having 2 to 60 carbon atoms.

In an exemplary embodiment of the present application, R9 and R10 are the same as or different from each other, and may be each independently a substituted or unsubstituted aryl group having 6 to 40 carbon atoms; or a substituted or unsubstituted heteroaryl group having 2 to 40 carbon atoms.

In an exemplary embodiment of the present application, R9 and R10 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; a substituted or unsubstituted triphenylenyl group; a substituted or unsubstituted fluorenyl group; a substituted or unsubstituted spirobifluorenyl group; a substituted or unsubstituted dibenzofuran group; or a substituted or unsubstituted dibenzothiophene group.

In an exemplary embodiment of the present application, R9 and R10 are the same as or different from each other, and may be each independently a phenyl group unsubstituted or substituted with one or more deuteriums; a biphenyl group unsubstituted or substituted with one or more deuteriums; a terphenyl group unsubstituted or substituted with one or more deuteriums; a naphthyl group unsubstituted or substituted with one or more deuteriums; a phenanthrenyl group unsubstituted or substituted with one or more deuteriums; a triphenylenyl group unsubstituted or substituted with one or more deuteriums; a fluorenyl group unsubstituted or substituted with one or more substituents selected from the group consisting of an alkyl group having 1 to 10 carbon atoms, an aryl group having 6 to 20 carbon atoms and deuterium; a spirobifluorenyl group unsubstituted or substituted with one or more deuteriums; a dibenzofuran group unsubstituted or substituted with one or more deuteriums; or a dibenzothiophene group unsubstituted or substituted with one or more deuteriums.

In an exemplary embodiment of the present application, R9 and R10 are the same as or different from each other, and are each independently a substituted or unsubstituted aryl group having 6 to 60 carbon atoms.

In an exemplary embodiment of the present application, R9 and R10 are the same as or different from each other, and are each independently a substituted or unsubstituted heteroaryl group having 2 to 60 carbon atoms.

In an exemplary embodiment of the present application, one of R9 and R10 is a substituted or unsubstituted aryl group having 6 to 60 carbon atoms, and the other is a substituted or unsubstituted heteroaryl group having 2 to 60 carbon atoms.

In an exemplary embodiment of the present application, Chemical Formula 1 may be represented by any one of the following Chemical Formulae 2 to 5.

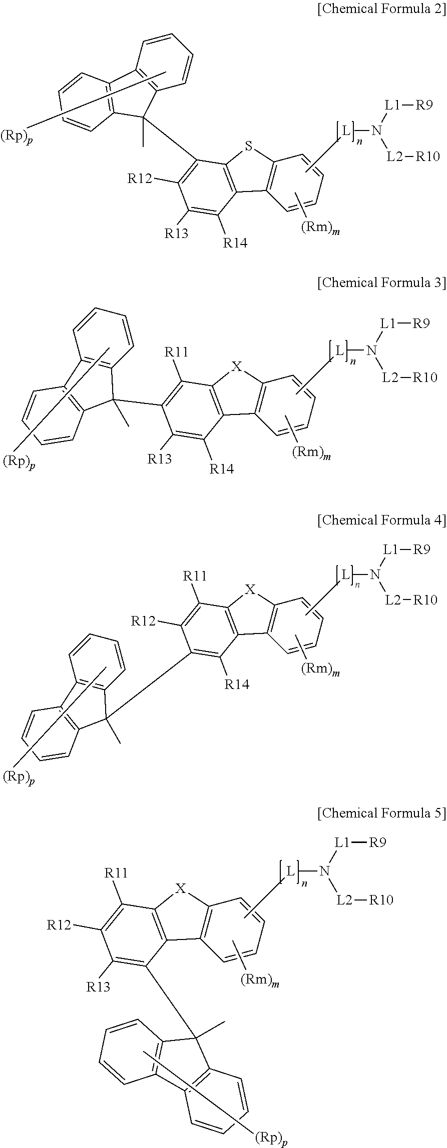

In Chemical Formulae 2 to 5,
R11 to R14 and Rm are the same as or different from each other, and are each independently hydrogen; deuterium; a halogen group; a cyano group; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms, m is an integer from 0 to 3, and when m is 2 or higher, Rm's in the parenthesis are the same as or different from each other, and the definitions of X, L, L1, L2, R9, R10, Rp, n and p are the same as those in Chemical Formula 1.

In an exemplary embodiment of the present application, R11 to R14 and Rm may be hydrogen; deuterium; a halogen group; a cyano group; a substituted or unsubstituted alkyl group having 1 to 40 carbon atoms; a substituted or unsubstituted aryl group having 6 to 40 carbon atoms; or a substituted or unsubstituted heteroaryl group having 2 to 40 carbon atoms.

In an exemplary embodiment of the present application, R11 to R14 and Rm may be hydrogen; deuterium; a halogen group; a cyano group; a substituted or unsubstituted alkyl group having 1 to 20 carbon atoms; a substituted or unsubstituted aryl group having 6 to 20 carbon atoms; or a substituted or unsubstituted heteroaryl group having 2 to 20 carbon atoms.

In an exemplary embodiment of the present application, R11 to R14 and Rm may be hydrogen; deuterium; a halogen group; or a cyano group.

In an exemplary embodiment of the present application, R11 to R14 and Rm are hydrogen; or deuterium.

In an exemplary embodiment of the present application, m is an integer from 0 to 3, and when m is 2 or higher, Ro's in the parenthesis are the same as or different from each other.

In an exemplary embodiment of the present application, m is 0.

In an exemplary embodiment of the present application, m is 1.

In an exemplary embodiment of the present application, m is 2.

In an exemplary embodiment of the present application, m is 3.

In an exemplary embodiment of the present application, when m is 2 or higher, Rm's in the parenthesis are the same as or different from each other.

In an exemplary embodiment of the present application, Chemical Formula 1 may be represented by any one of the following Chemical Formulae 6 to 9.

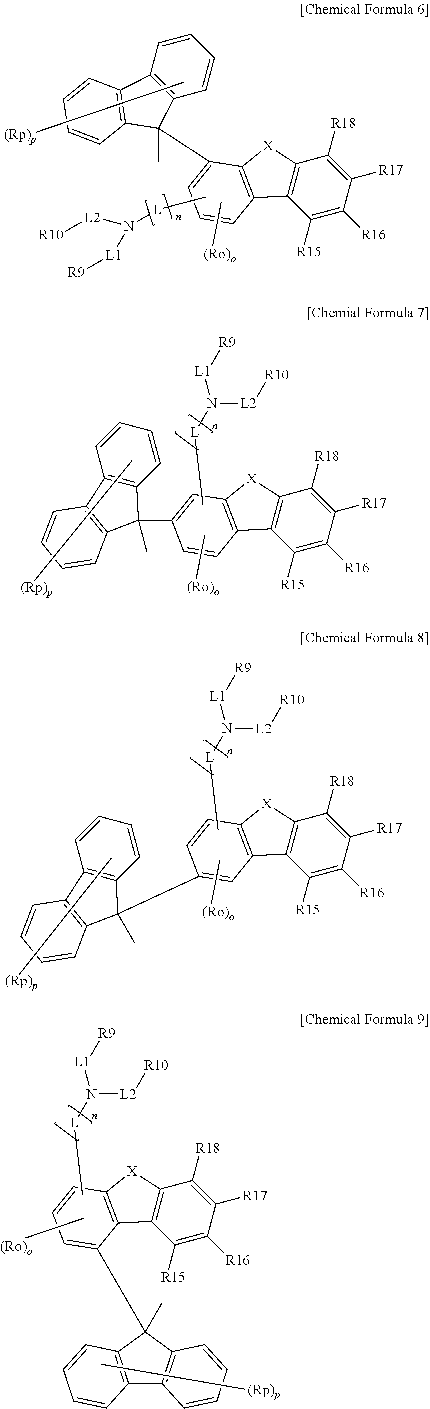

In Chemical Formulae 6 to 9,
R15 to R18 and Ro are the same as or different from each other, and are each independently hydrogen; deuterium; a halogen group; a cyano group; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms, o is an integer from 0 to 3, and when o is 2 or higher, Ro's in the parenthesis are the same as or different from each other, and
the definitions of X, L, L1, L2, R9, R10, Rp, n and p are the same as those in Chemical Formula 1.

In an exemplary embodiment of the present application, R15 to R18 and Ro may be hydrogen; deuterium; a halogen group; a cyano group; a substituted or unsubstituted alkyl group having 1 to 40 carbon atoms; a substituted or unsubstituted aryl group having 6 to 40 carbon atoms; or a substituted or unsubstituted heteroaryl group having 2 to 40 carbon atoms.

In an exemplary embodiment of the present application, R15 to R18 and Ro may be hydrogen; deuterium; a halogen group; a cyano group; a substituted or unsubstituted alkyl group having 1 to 20 carbon atoms; a substituted or unsubstituted aryl group having 6 to 20 carbon atoms; or a substituted or unsubstituted heteroaryl group having 2 to 20 carbon atoms.

In an exemplary embodiment of the present application, R15 to R18 and Ro may be hydrogen; deuterium; a halogen group; or a cyano group.

In an exemplary embodiment of the present application, R15 to R18 and Ro are hydrogen; or deuterium.

In an exemplary embodiment of the present application, o is an integer from 0 to 3, and when o is 2 or higher, Ro's in the parenthesis are the same as or different from each other.

In an exemplary embodiment of the present application, o is 0.

In an exemplary embodiment of the present application, o is 1.

In an exemplary embodiment of the present application, o is 2.

In an exemplary embodiment of the present application, o is 3.

In an exemplary embodiment of the present application, when o is 2 or higher, Ro's in the parenthesis are the same as or different from each other.

According to an exemplary embodiment of the present application, Chemical Formula 1 may be represented by any one of the following compounds, but is not limited thereto.

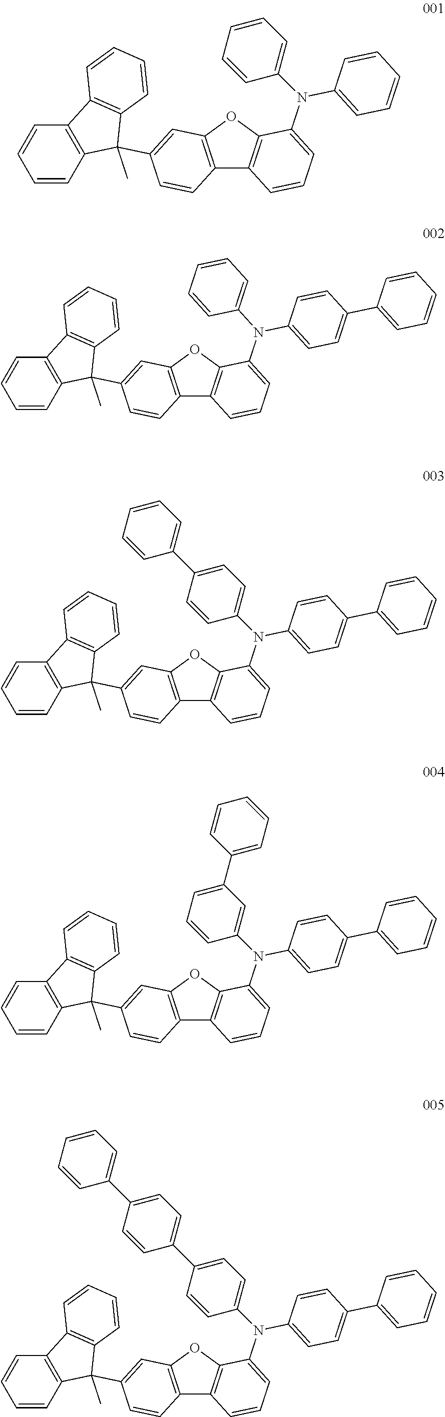 [SMILES: CC1(c2ccc3c(c2)oc2c(N(c4ccccc4)c4ccccc4)cccc23)c2ccccc2-c2ccccc21 ; CC1(c2ccc3c(c2)oc2c(N(c4ccccc4)c4ccc(-c5ccccc5)cc4)cccc23)c2ccccc2-c2ccccc21 ; CC1(c2ccc3c(c2)oc2c(N(c4ccc(-c5ccccc5)cc4)c4ccc(-c5ccccc5)cc4)cccc23)c2ccccc2-c2ccccc21 ; CC1(c2ccc3c(c2)oc2c(N(c4ccc(-c5ccccc5)cc4)c4cccc(-c5ccccc5)c4)cccc23)c2ccccc2-c2ccccc21 ; CC1(c2ccc3c(c2)oc2c(N(c4ccc(-c5ccccc5)cc4)c4ccc(-c5ccc(-c6ccccc6)cc5)cc4)cccc23)c2ccccc2-c2ccccc21]

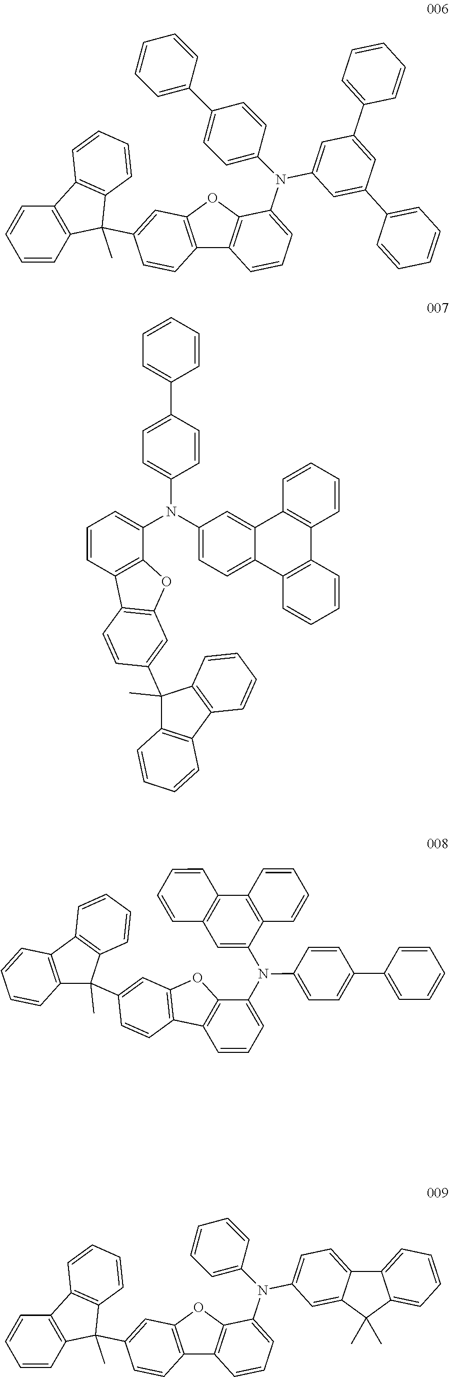 [SMILES: CC1(c2ccc3c(c2)oc2c(N(c4ccc(-c5ccccc5)cc4)c4cc(-c5ccccc5)cc(-c5ccccc5)c4)cccc23)c2ccccc2-c2ccccc21 ; CC1(c2ccc3c(c2)oc2c(N(c4ccc(-c5ccccc5)cc4)c4ccc5c6ccccc6c6ccccc6c5c4)cccc23)c2ccccc2-c2ccccc21 ; CC1(c2ccc3c(c2)oc2c(N(c4ccc(-c5ccccc5)cc4)c4cc5ccccc5c5ccccc45)cccc23)c2ccccc2-c2ccccc21 ; CC1(C)c2ccccc2-c2ccc(N(c3ccccc3)c3cccc4c3oc3cc(C5(C)c6ccccc6-c6ccccc65)ccc34)cc21]

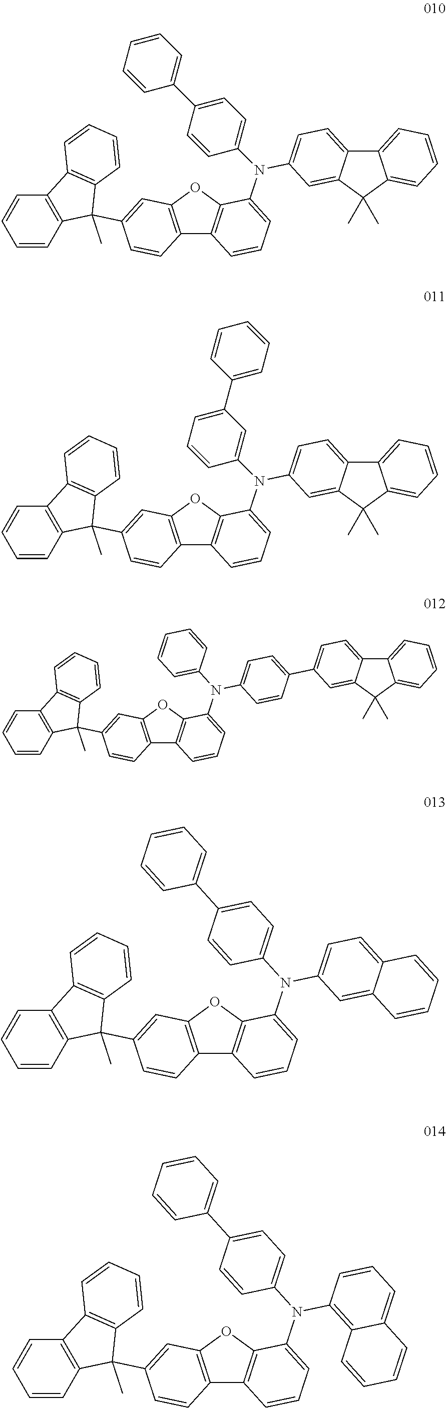 [SMILES: CC1(C)c2ccccc2-c2ccc(N(c3ccc(-c4ccccc4)cc3)c3cccc4c3oc3cc(C5(C)c6ccccc6-c6ccccc65)ccc34)cc21 ; CC1(C)c2ccccc2-c2ccc(N(c3cccc(-c4ccccc4)c3)c3cccc4c3oc3cc(C5(C)c6ccccc6-c6ccccc65)ccc34)cc21 ; CC1(C)c2ccccc2-c2ccc(-c3ccc(N(c4ccccc4)c4cccc5c4oc4cc(C6(C)c7ccccc7-c7ccccc76)ccc45)cc3)cc21 ; CC1(c2ccc3c(c2)oc2c(N(c4ccc(-c5ccccc5)cc4)c4ccc5ccccc5c4)cccc23)c2ccccc2-c2ccccc21 ; CC1(c2ccc3c(c2)oc2c(N(c4ccc(-c5ccccc5)cc4)c4cccc5ccccc45)cccc23)c2ccccc2-c2ccccc21]

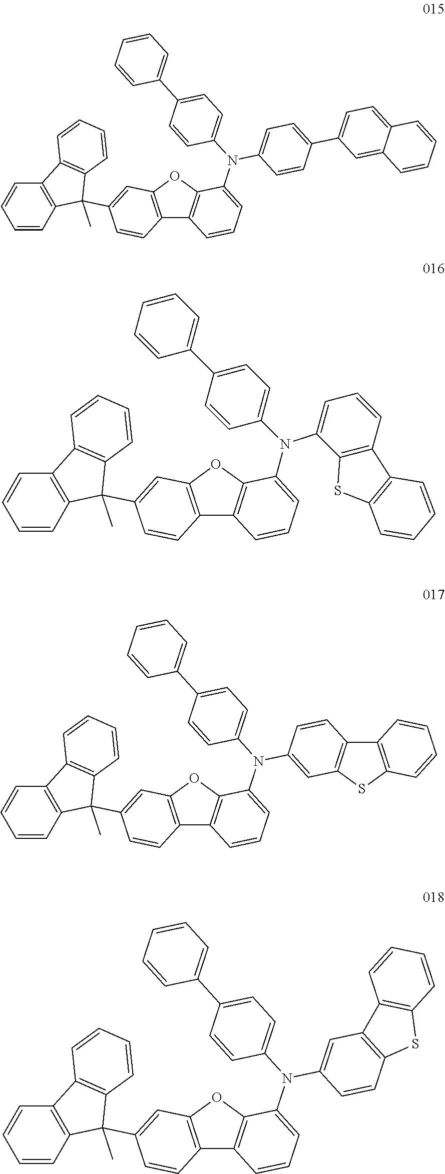 [SMILES: CC1(c2ccc3c(c2)oc2c(N(c4ccc(-c5ccccc5)cc4)c4ccc(-c5ccc6ccccc6c5)cc4)cccc23)c2ccccc2-c2ccccc21 ; CC1(c2ccc3c(c2)oc2c(N(c4ccc(-c5ccccc5)cc4)c4cccc5c4sc4ccccc45)cccc23)c2ccccc2-c2ccccc21 ; CC1(c2ccc3c(c2)oc2c(N(c4ccc(-c5ccccc5)cc4)c4ccc5c(c4)sc4ccccc45)cccc23)c2ccccc2-c2ccccc21 ; CC1(c2ccc3c(c2)oc2c(N(c4ccc(-c5ccccc5)cc4)c4ccc5sc6ccccc6c5c4)cccc23)c2ccccc2-c2ccccc21]

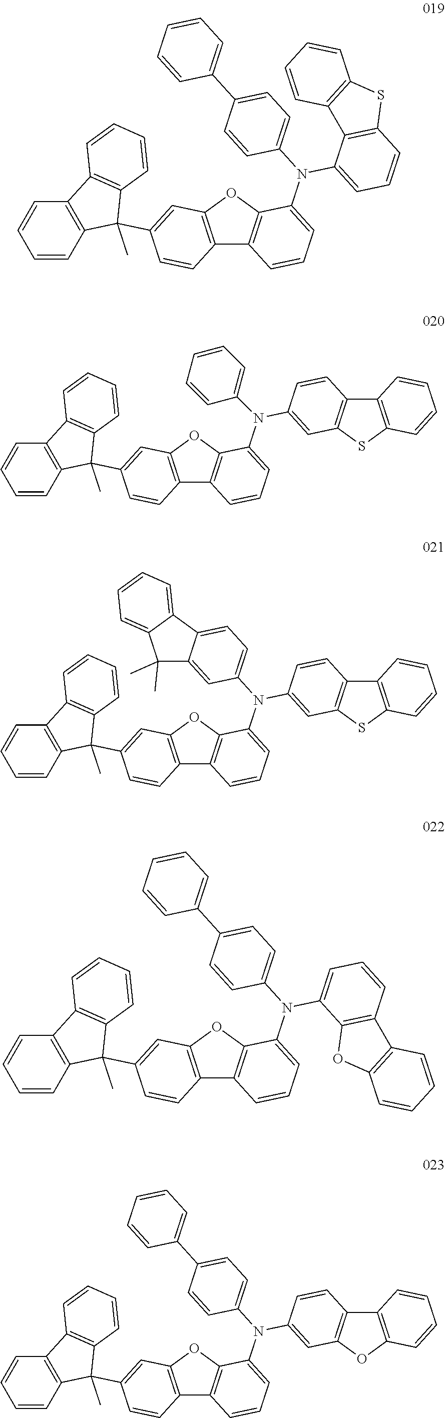 [SMILES: CC1(c2ccc3c(c2)oc2c(N(c4ccc(-c5ccccc5)cc4)c4cccc5sc6ccccc6c45)cccc23)c2ccccc2-c2ccccc21 ; CC1(c2ccc3c(c2)oc2c(N(c4ccccc4)c4ccc5c(c4)sc4ccccc45)cccc23)c2ccccc2-c2ccccc21 ; CC1(C)c2ccccc2-c2ccc(N(c3ccc4c(c3)sc3ccccc34)c3cccc4c3oc3cc(C5(C)c6ccccc6-c6ccccc65)ccc34)cc21 ; CC1(c2ccc3c(c2)oc2c(N(c4ccc(-c5ccccc5)cc4)c4cccc5c4oc4ccccc45)cccc23)c2ccccc2-c2ccccc21 ; CC1(c2ccc3c(c2)oc2c(N(c4ccc(-c5ccccc5)cc4)c4ccc5c(c4)oc4ccccc45)cccc23)c2ccccc2-c2ccccc21]

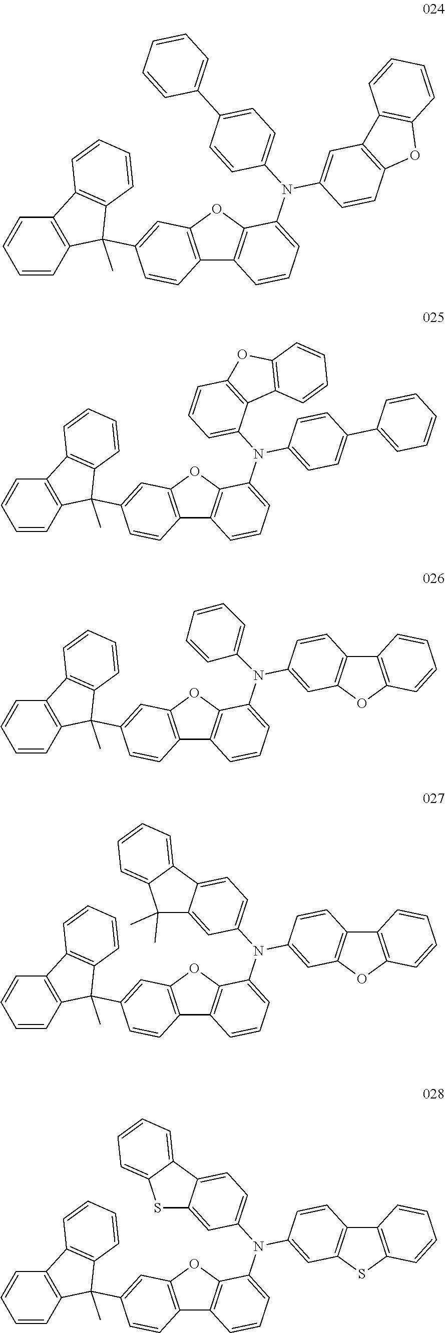 [SMILES: CC1(c2ccc3c(c2)oc2c(N(c4ccc(-c5ccccc5)cc4)c4ccc5oc6ccccc6c5c4)cccc23)c2ccccc2-c2ccccc21 ; CC1(c2ccc3c(c2)oc2c(N(c4ccc(-c5ccccc5)cc4)c4cccc5oc6ccccc6c45)cccc23)c2ccccc2-c2ccccc21 ; CC1(c2ccc3c(c2)oc2c(N(c4ccccc4)c4ccc5c(c4)oc4ccccc45)cccc23)c2ccccc2-c2ccccc21 ; CC1(C)c2ccccc2-c2ccc(N(c3ccc4c(c3)oc3ccccc34)c3cccc4c3oc3cc(C5(C)c6ccccc6-c6ccccc65)ccc34)cc21 ; CC1(c2ccc3c(c2)oc2c(N(c4ccc5c(c4)sc4ccccc45)c4ccc5c(c4)sc4ccccc45)cccc23)c2ccccc2-c2ccccc21]

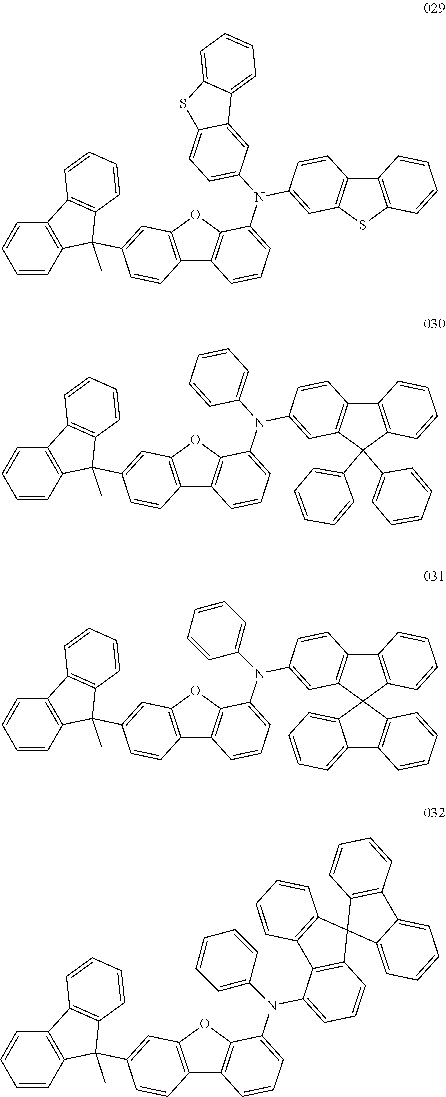 [SMILES: CC1(c2ccc3c(c2)oc2c(N(c4ccc5c(c4)sc4ccccc45)c4ccc5sc6ccccc6c5c4)cccc23)c2ccccc2-c2ccccc21 ; CC1(c2ccc3c(c2)oc2c(N(c4ccccc4)c4ccc5c(c4)C(c4ccccc4)(c4ccccc4)c4ccccc4-5)cccc23)c2ccccc2-c2ccccc21 ; CC1(c2ccc3c(c2)oc2c(N(c4ccccc4)c4ccc5c(c4)C4(c6ccccc6-c6ccccc64)c4ccccc4-5)cccc23)c2ccccc2-c2ccccc21 ; CC1(c2ccc3c(c2)oc2c(N(c4ccccc4)c4cccc5c4-c4ccccc4C54c5ccccc5-c5ccccc54)cccc23)c2ccccc2-c2ccccc21]

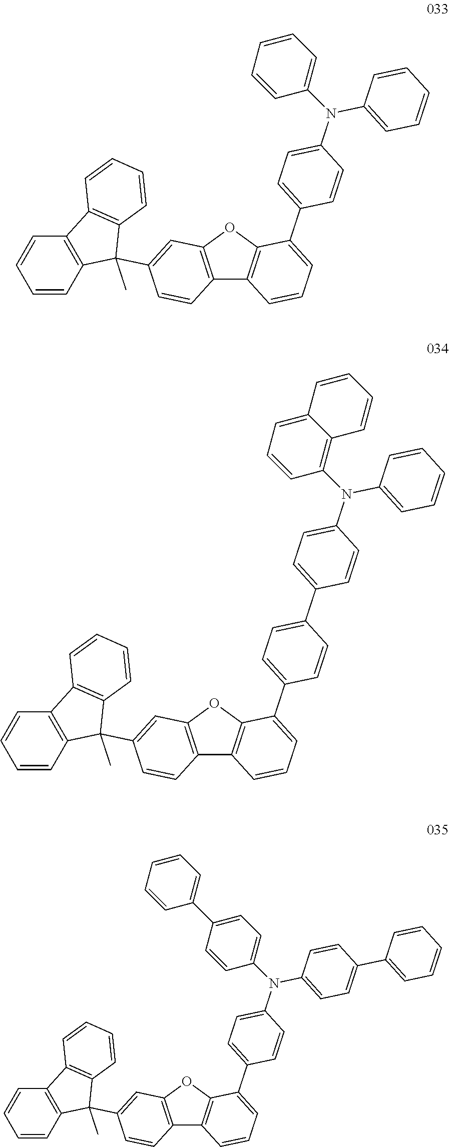 [SMILES: CC1(c2ccc3c(c2)oc2c(-c4ccc(N(c5ccccc5)c5ccccc5)cc4)cccc23)c2ccccc2-c2ccccc21 ; CC1(c2ccc3c(c2)oc2c(-c4ccc(-c5ccc(N(c6ccccc6)c6cccc7ccccc67)cc5)cc4)cccc23)c2ccccc2-c2ccccc21 ; CC1(c2ccc3c(c2)oc2c(-c4ccc(N(c5ccc(-c6ccccc6)cc5)c5ccc(-c6ccccc6)cc5)cc4)cccc23)c2ccccc2-c2ccccc21]

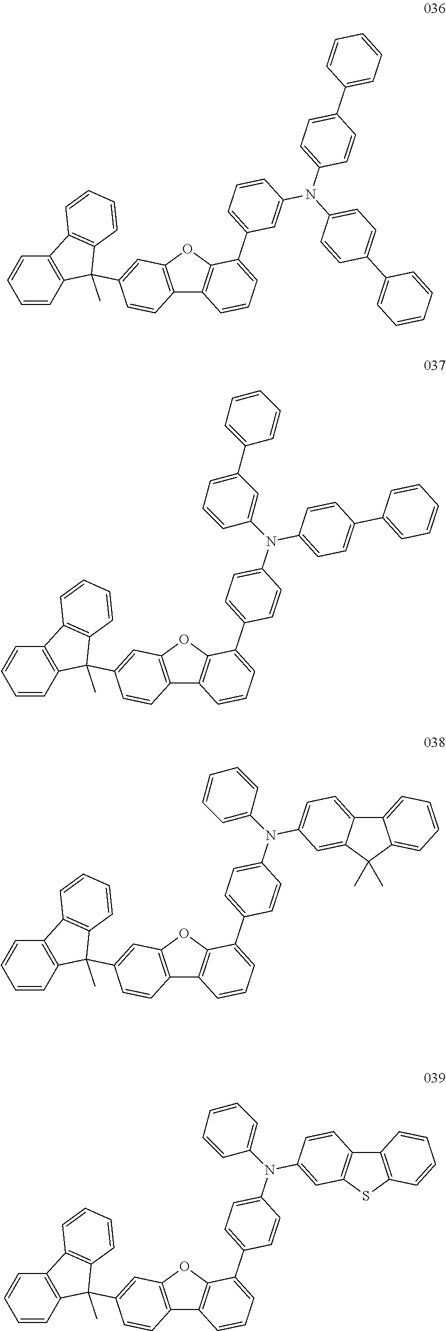 [SMILES: CC1(c2ccc3c(c2)oc2c(-c4cccc(N(c5ccc(-c6ccccc6)cc5)c5ccc(-c6ccccc6)cc5)c4)cccc23)c2ccccc2-c2ccccc21 ; CC1(c2ccc3c(c2)oc2c(-c4ccc(N(c5ccc(-c6ccccc6)cc5)c5cccc(-c6ccccc6)c5)cc4)cccc23)c2ccccc2-c2ccccc21 ; CC1(C)c2ccccc2-c2ccc(N(c3ccccc3)c3ccc(-c4cccc5c4oc4cc(C6(C)c7ccccc7-c7ccccc76)ccc45)cc3)cc21 ; CC1(c2ccc3c(c2)oc2c(-c4ccc(N(c5ccccc5)c5ccc6c(c5)sc5ccccc56)cc4)cccc23)c2ccccc2-c2ccccc21]

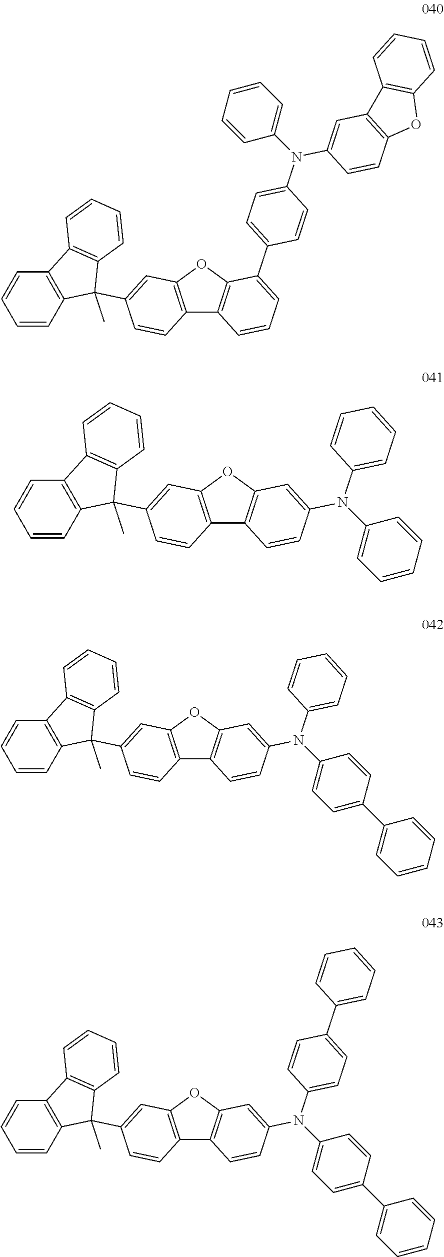 [SMILES: CC1(c2ccc3c(c2)oc2c(-c4ccc(N(c5ccccc5)c5ccc6oc7ccccc7c6c5)cc4)cccc23)c2ccccc2-c2ccccc21 ; CC1(c2ccc3c(c2)oc2cc(N(c4ccccc4)c4ccccc4)ccc23)c2ccccc2-c2ccccc21 ; CC1(c2ccc3c(c2)oc2cc(N(c4ccccc4)c4ccc(-c5ccccc5)cc4)ccc23)c2ccccc2-c2ccccc21 ; CC1(c2ccc3c(c2)oc2cc(N(c4ccc(-c5ccccc5)cc4)c4ccc(-c5ccccc5)cc4)ccc23)c2ccccc2-c2ccccc21]

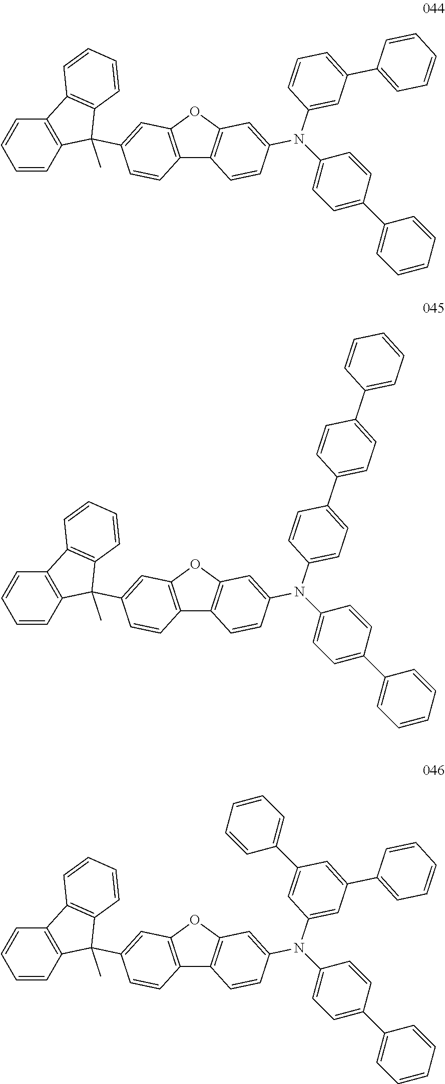 [SMILES: CC1(c2ccc3c(c2)oc2cc(N(c4ccc(-c5ccccc5)cc4)c4cccc(-c5ccccc5)c4)ccc23)c2ccccc2-c2ccccc21 ; CC1(c2ccc3c(c2)oc2cc(N(c4ccc(-c5ccccc5)cc4)c4ccc(-c5ccc(-c6ccccc6)cc5)cc4)ccc23)c2ccccc2-c2ccccc21 ; CC1(c2ccc3c(c2)oc2cc(N(c4ccc(-c5ccccc5)cc4)c4cc(-c5ccccc5)cc(-c5ccccc5)c4)ccc23)c2ccccc2-c2ccccc21]

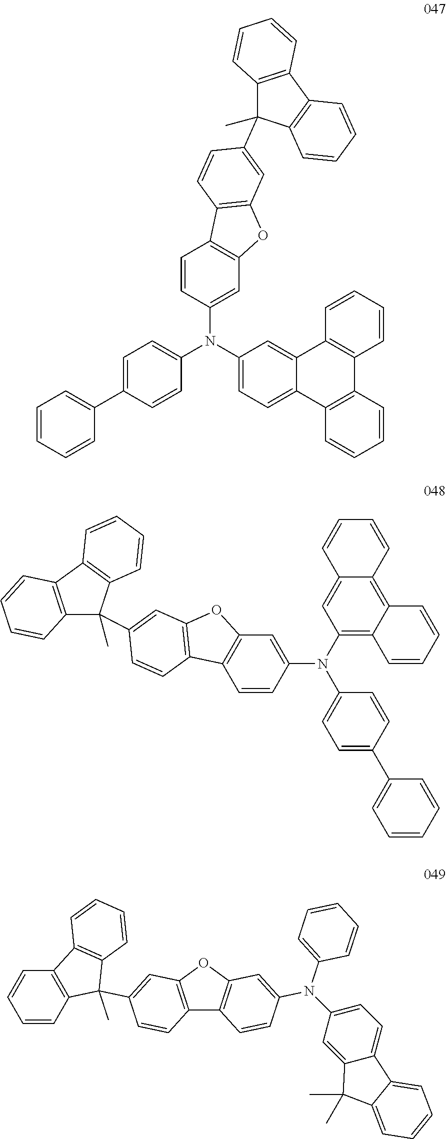 [SMILES: CC1(c2ccc3c(c2)oc2cc(N(c4ccc(-c5ccccc5)cc4)c4ccc5c6ccccc6c6ccccc6c5c4)ccc23)c2ccccc2-c2ccccc21 ; CC1(c2ccc3c(c2)oc2cc(N(c4ccc(-c5ccccc5)cc4)c4cc5ccccc5c5ccccc45)ccc23)c2ccccc2-c2ccccc21 ; CC1(C)c2ccccc2-c2ccc(N(c3ccccc3)c3ccc4c(c3)oc3cc(C5(C)c6ccccc6-c6ccccc65)ccc34)cc21]

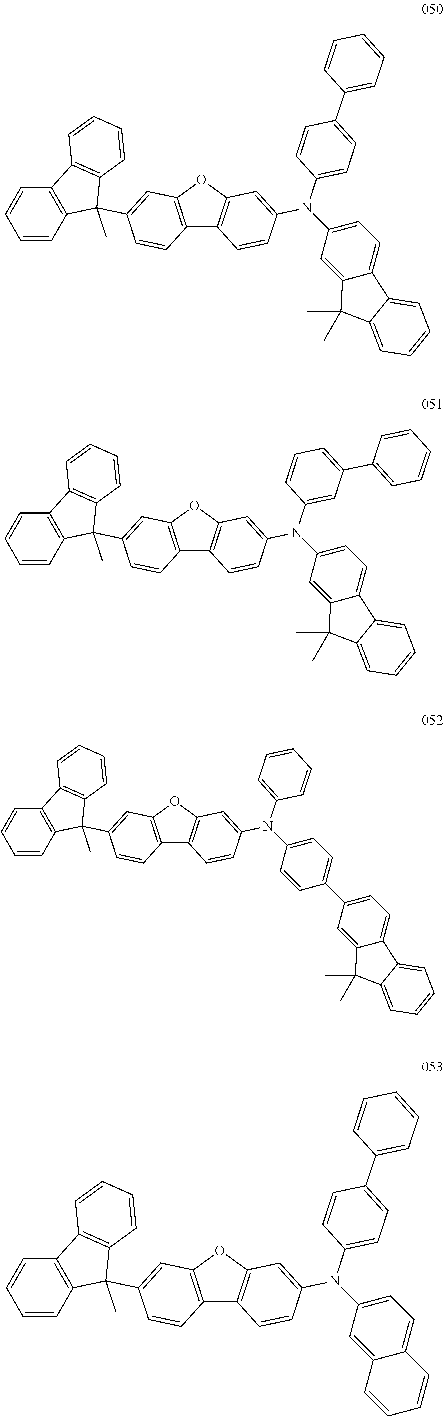 [SMILES: CC1(C)c2ccccc2-c2ccc(N(c3ccc(-c4ccccc4)cc3)c3ccc4c(c3)oc3cc(C5(C)c6ccccc6-c6ccccc65)ccc34)cc21 ; CC1(C)c2ccccc2-c2ccc(N(c3cccc(-c4ccccc4)c3)c3ccc4c(c3)oc3cc(C5(C)c6ccccc6-c6ccccc65)ccc34)cc21 ; CC1(C)c2ccccc2-c2ccc(-c3ccc(N(c4ccccc4)c4ccc5c(c4)oc4cc(C6(C)c7ccccc7-c7ccccc76)ccc45)cc3)cc21 ; CC1(c2ccc3c(c2)oc2cc(N(c4ccc(-c5ccccc5)cc4)c4ccc5ccccc5c4)ccc23)c2ccccc2-c2ccccc21]

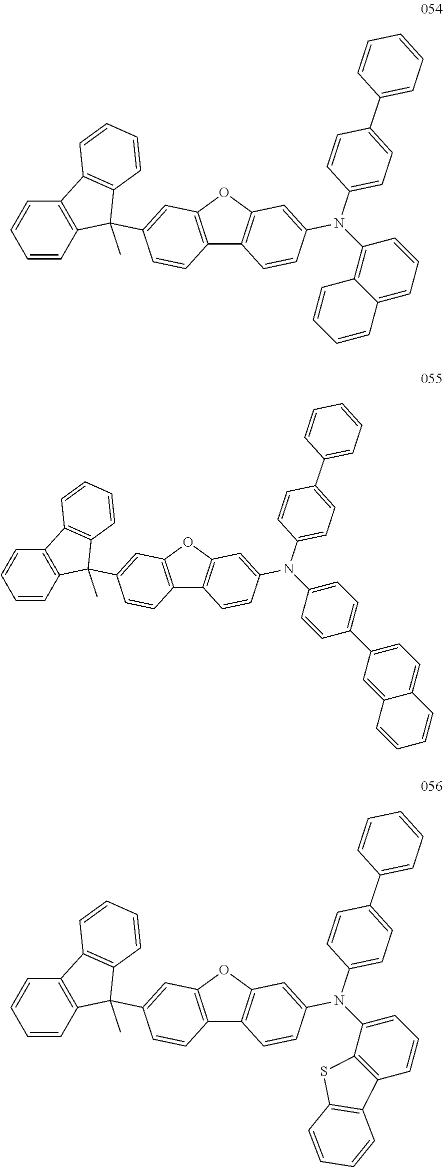 [SMILES: CC1(c2ccc3c(c2)oc2cc(N(c4ccc(-c5ccccc5)cc4)c4cccc5ccccc45)ccc23)c2ccccc2-c2ccccc21 ; CC1(c2ccc3c(c2)oc2cc(N(c4ccc(-c5ccccc5)cc4)c4ccc(-c5ccc6ccccc6c5)cc4)ccc23)c2ccccc2-c2ccccc21 ; CC1(c2ccc3c(c2)oc2cc(N(c4ccc(-c5ccccc5)cc4)c4cccc5c4sc4ccccc45)ccc23)c2ccccc2-c2ccccc21]

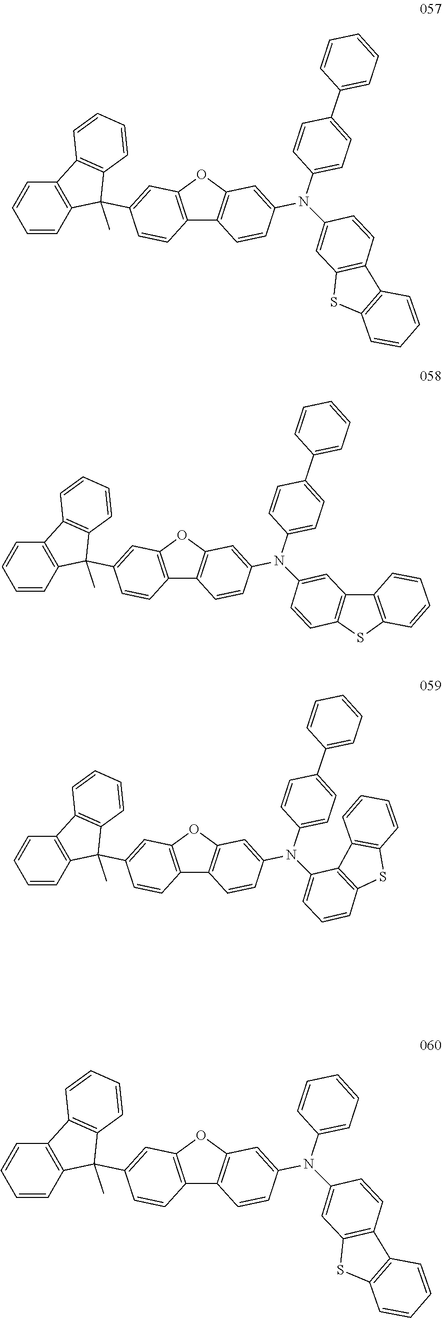 [SMILES: CC1(c2ccc3c(c2)oc2cc(N(c4ccc(-c5ccccc5)cc4)c4ccc5c(c4)sc4ccccc45)ccc23)c2ccccc2-c2ccccc21 ; CC1(c2ccc3c(c2)oc2cc(N(c4ccc(-c5ccccc5)cc4)c4ccc5sc6ccccc6c5c4)ccc23)c2ccccc2-c2ccccc21 ; CC1(c2ccc3c(c2)oc2cc(N(c4ccc(-c5ccccc5)cc4)c4cccc5sc6ccccc6c45)ccc23)c2ccccc2-c2ccccc21 ; CC1(c2ccc3c(c2)oc2cc(N(c4ccccc4)c4ccc5c(c4)sc4ccccc45)ccc23)c2ccccc2-c2ccccc21]

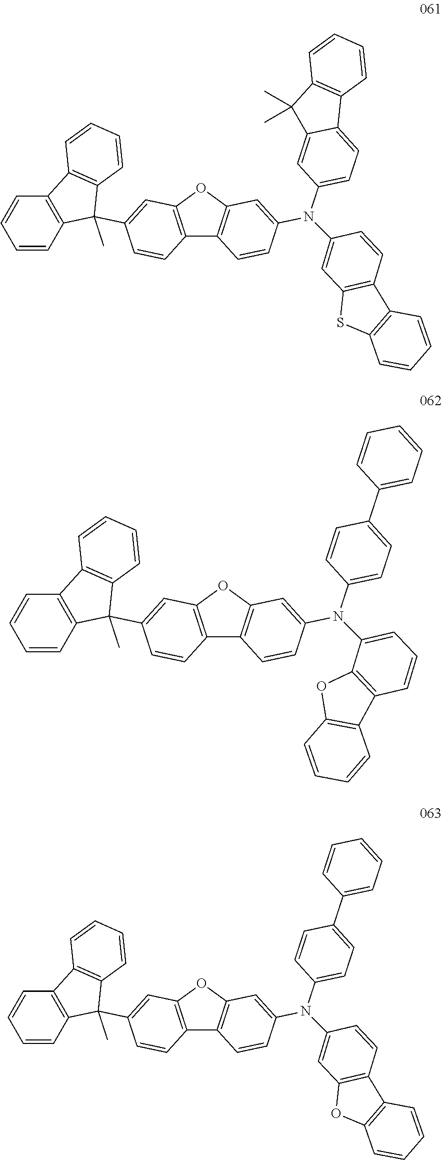 [SMILES: CC1(C)c2ccccc2-c2ccc(N(c3ccc4c(c3)oc3cc(C5(C)c6ccccc6-c6ccccc65)ccc34)c3ccc4c(c3)sc3ccccc34)cc21 ; CC1(c2ccc3c(c2)oc2cc(N(c4ccc(-c5ccccc5)cc4)c4cccc5c4oc4ccccc45)ccc23)c2ccccc2-c2ccccc21 ; CC1(c2ccc3c(c2)oc2cc(N(c4ccc(-c5ccccc5)cc4)c4ccc5c(c4)oc4ccccc45)ccc23)c2ccccc2-c2ccccc21]

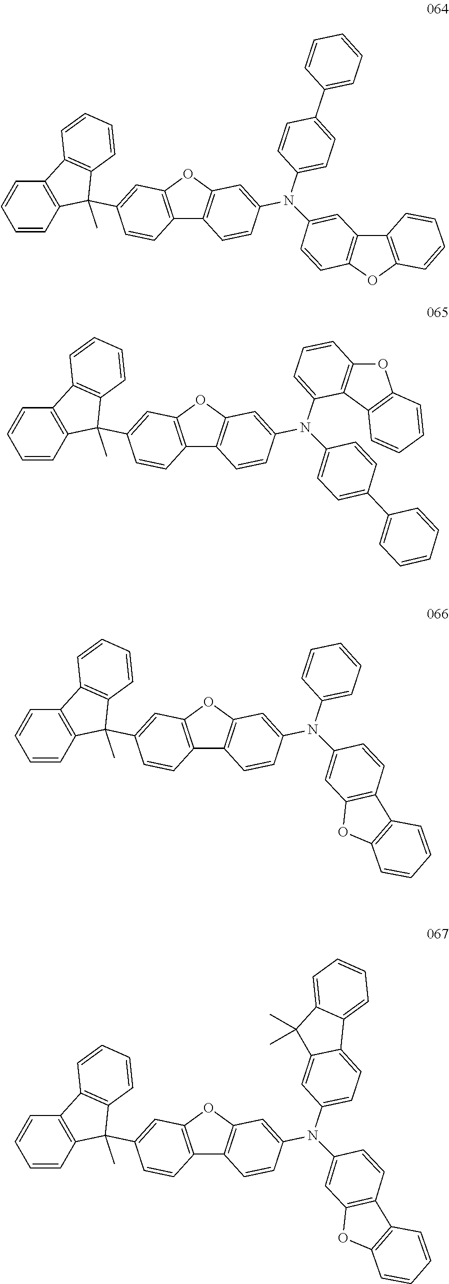 [SMILES: CC1(c2ccc3c(c2)oc2cc(N(c4ccc(-c5ccccc5)cc4)c4ccc5oc6ccccc6c5c4)ccc23)c2ccccc2-c2ccccc21 ; CC1(c2ccc3c(c2)oc2cc(N(c4ccc(-c5ccccc5)cc4)c4cccc5oc6ccccc6c45)ccc23)c2ccccc2-c2ccccc21 ; CC1(c2ccc3c(c2)oc2cc(N(c4ccccc4)c4ccc5c(c4)oc4ccccc45)ccc23)c2ccccc2-c2ccccc21 ; CC1(C)c2ccccc2-c2ccc(N(c3ccc4c(c3)oc3ccccc34)c3ccc4c(c3)oc3cc(C5(C)c6ccccc6-c6ccccc65)ccc34)cc21]

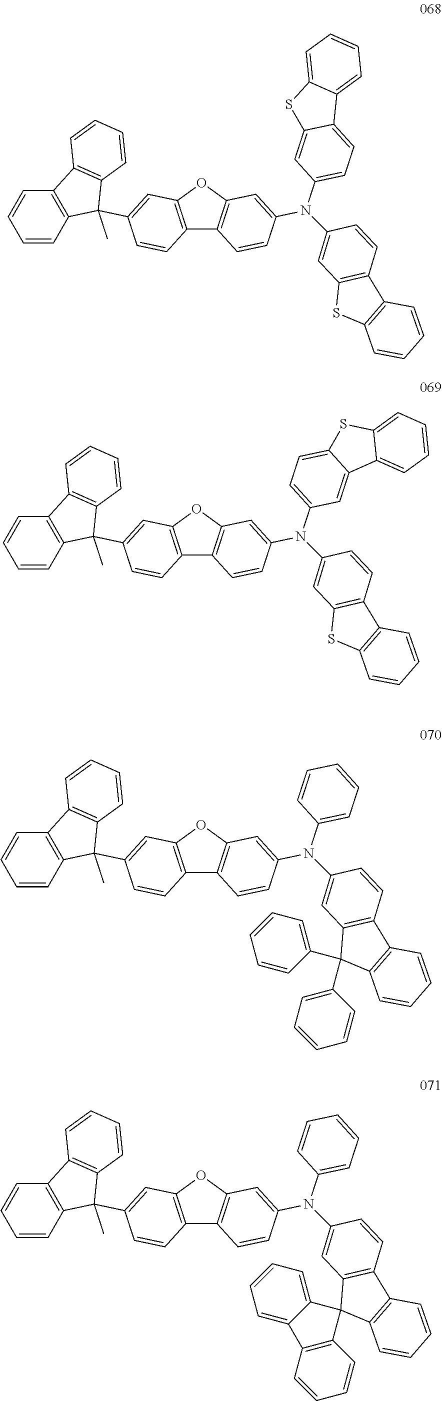 [SMILES: CC1(c2ccc3c(c2)oc2cc(N(c4ccc5c(c4)sc4ccccc45)c4ccc5c(c4)sc4ccccc45)ccc23)c2ccccc2-c2ccccc21 ; CC1(c2ccc3c(c2)oc2cc(N(c4ccc5c(c4)sc4ccccc45)c4ccc5sc6ccccc6c5c4)ccc23)c2ccccc2-c2ccccc21 ; CC1(c2ccc3c(c2)oc2cc(N(c4ccccc4)c4ccc5c(c4)C(c4ccccc4)(c4ccccc4)c4ccccc4-5)ccc23)c2ccccc2-c2ccccc21 ; CC1(c2ccc3c(c2)oc2cc(N(c4ccccc4)c4ccc5c(c4)C4(c6ccccc6-c6ccccc64)c4ccccc4-5)ccc23)c2ccccc2-c2ccccc21]

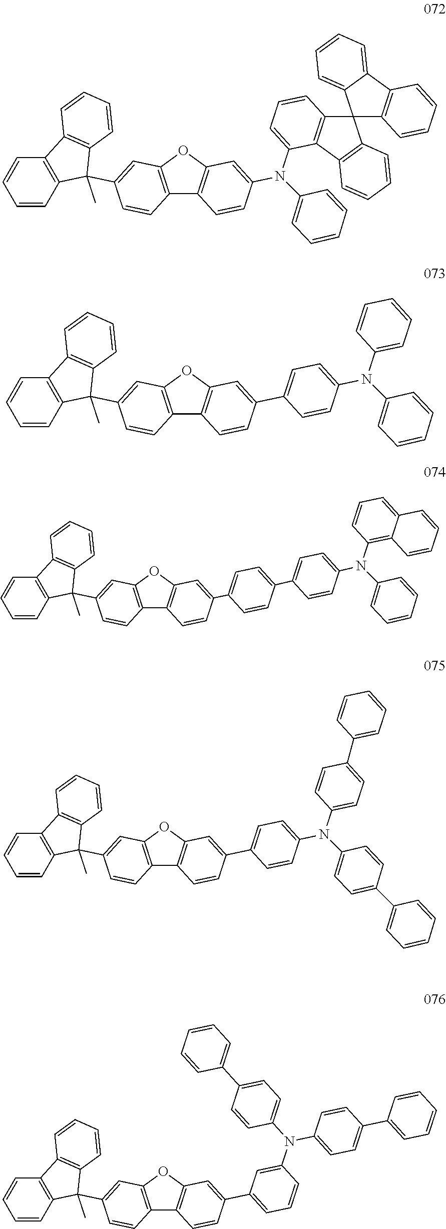 [SMILES: CC1(c2ccc3c(c2)oc2cc(N(c4ccccc4)c4cccc5c4-c4ccccc4C54c5ccccc5-c5ccccc54)ccc23)c2ccccc2-c2ccccc21 ; CC1(c2ccc3c(c2)oc2cc(-c4ccc(N(c5ccccc5)c5ccccc5)cc4)ccc23)c2ccccc2-c2ccccc21 ; CC1(c2ccc3c(c2)oc2cc(-c4ccc(-c5ccc(N(c6ccccc6)c6cccc7ccccc67)cc5)cc4)ccc23)c2ccccc2-c2ccccc21 ; CC1(c2ccc3c(c2)oc2cc(-c4ccc(N(c5ccc(-c6ccccc6)cc5)c5ccc(-c6ccccc6)cc5)cc4)ccc23)c2ccccc2-c2ccccc21 ; CC1(c2ccc3c(c2)oc2cc(-c4cccc(N(c5ccc(-c6ccccc6)cc5)c5ccc(-c6ccccc6)cc5)c4)ccc23)c2ccccc2-c2ccccc21]

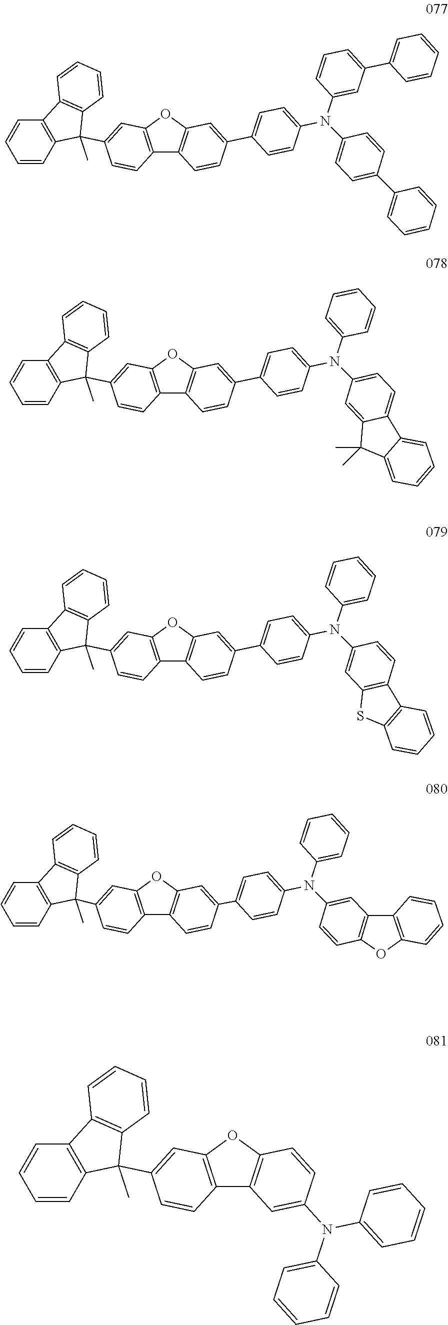 [SMILES: CC1(c2ccc3c(c2)oc2cc(-c4ccc(N(c5ccc(-c6ccccc6)cc5)c5cccc(-c6ccccc6)c5)cc4)ccc23)c2ccccc2-c2ccccc21 ; CC1(C)c2ccccc2-c2ccc(N(c3ccccc3)c3ccc(-c4ccc5c(c4)oc4cc(C6(C)c7ccccc7-c7ccccc76)ccc45)cc3)cc21 ; CC1(c2ccc3c(c2)oc2cc(-c4ccc(N(c5ccccc5)c5ccc6c(c5)sc5ccccc56)cc4)ccc23)c2ccccc2-c2ccccc21 ; CC1(c2ccc3c(c2)oc2cc(-c4ccc(N(c5ccccc5)c5ccc6oc7ccccc7c6c5)cc4)ccc23)c2ccccc2-c2ccccc21 ; CC1(c2ccc3c(c2)oc2ccc(N(c4ccccc4)c4ccccc4)cc23)c2ccccc2-c2ccccc21]

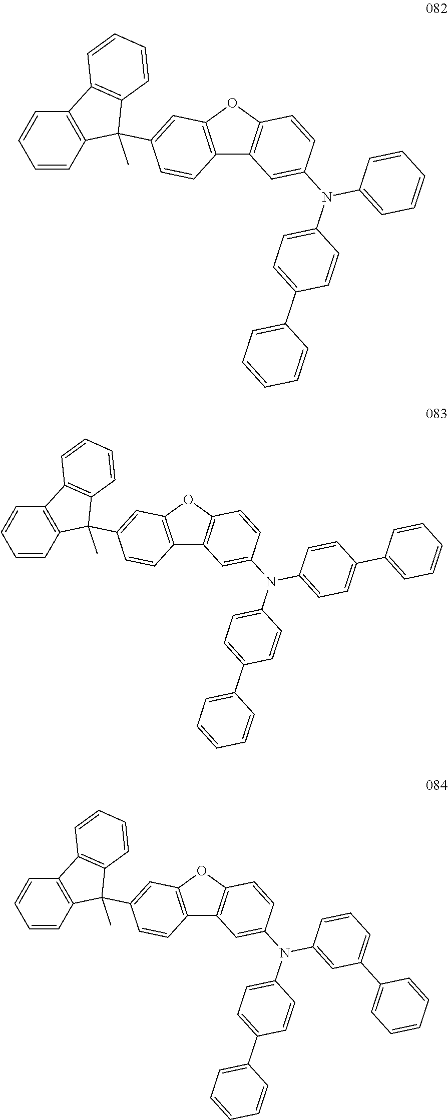 [SMILES: CC1(c2ccc3c(c2)oc2ccc(N(c4ccccc4)c4ccc(-c5ccccc5)cc4)cc23)c2ccccc2-c2ccccc21 ; CC1(c2ccc3c(c2)oc2ccc(N(c4ccc(-c5ccccc5)cc4)c4ccc(-c5ccccc5)cc4)cc23)c2ccccc2-c2ccccc21 ; CC1(c2ccc3c(c2)oc2ccc(N(c4ccc(-c5ccccc5)cc4)c4cccc(-c5ccccc5)c4)cc23)c2ccccc2-c2ccccc21]

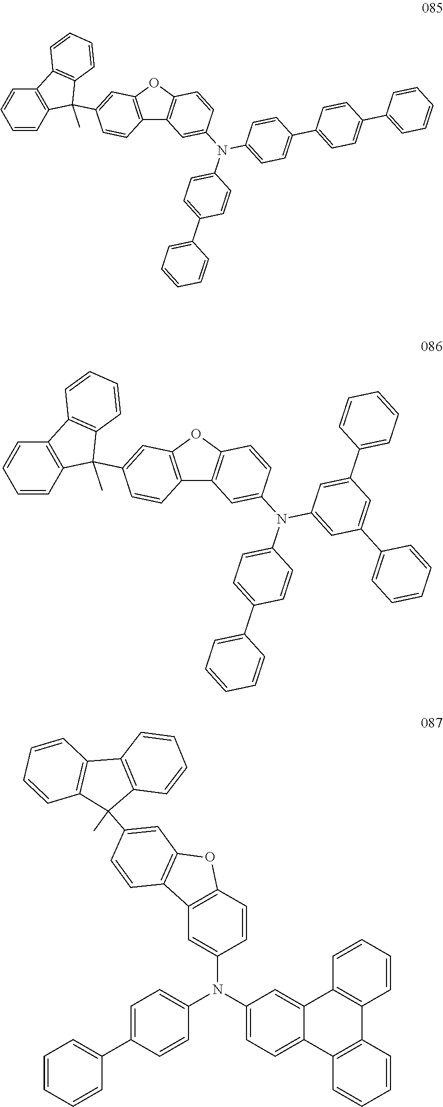 [SMILES: CC1(c2ccc3c(c2)oc2ccc(N(c4ccc(-c5ccccc5)cc4)c4ccc(-c5ccc(-c6ccccc6)cc5)cc4)cc23)c2ccccc2-c2ccccc21 ; CC1(c2ccc3c(c2)oc2ccc(N(c4ccc(-c5ccccc5)cc4)c4cc(-c5ccccc5)cc(-c5ccccc5)c4)cc23)c2ccccc2-c2ccccc21 ; CC1(c2ccc3c(c2)oc2ccc(N(c4ccc(-c5ccccc5)cc4)c4ccc5c6ccccc6c6ccccc6c5c4)cc23)c2ccccc2-c2ccccc21]

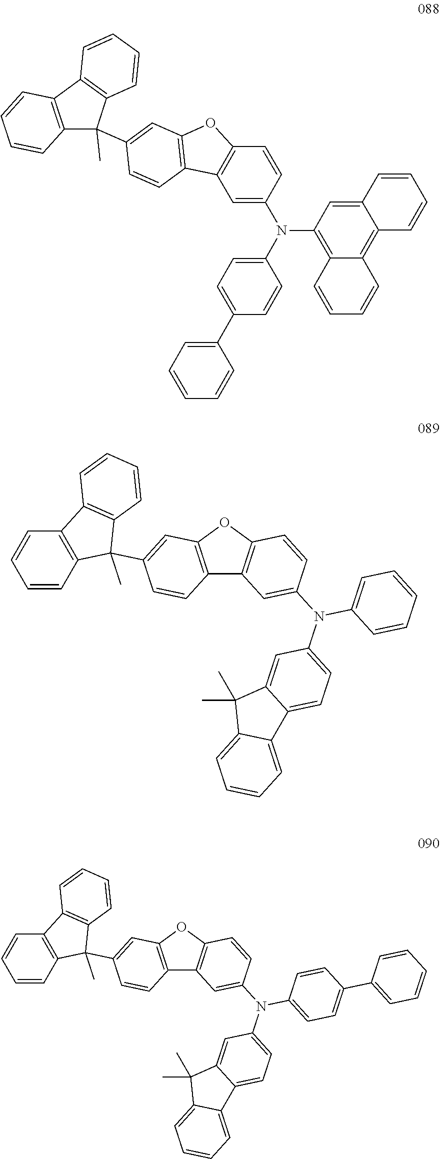 [SMILES: CC1(c2ccc3c(c2)oc2ccc(N(c4ccc(-c5ccccc5)cc4)c4cc5ccccc5c5ccccc45)cc23)c2ccccc2-c2ccccc21 ; CC1(C)c2ccccc2-c2ccc(N(c3ccccc3)c3ccc4oc5cc(C6(C)c7ccccc7-c7ccccc76)ccc5c4c3)cc21 ; CC1(C)c2ccccc2-c2ccc(N(c3ccc(-c4ccccc4)cc3)c3ccc4oc5cc(C6(C)c7ccccc7-c7ccccc76)ccc5c4c3)cc21]

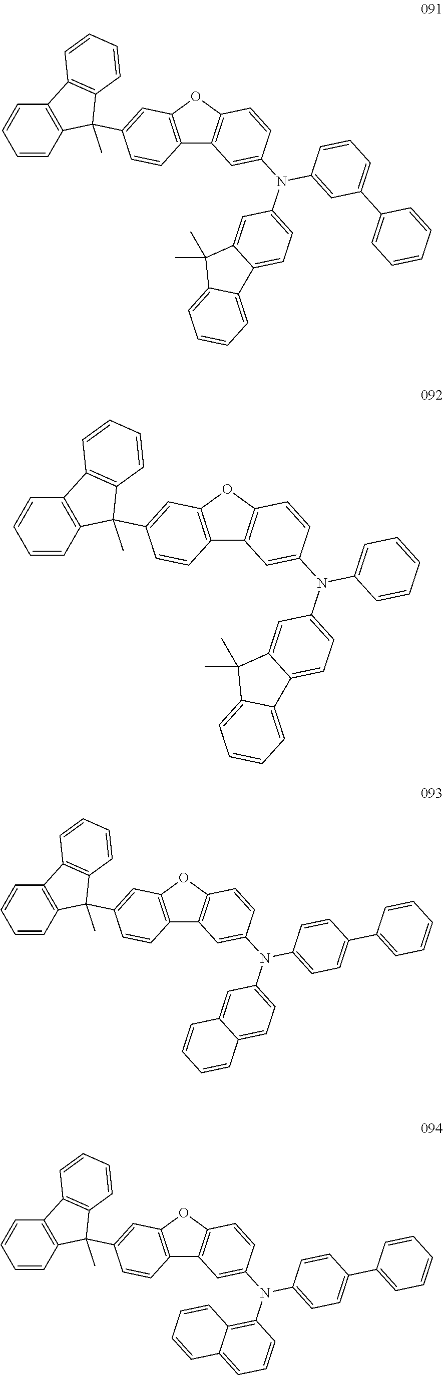 [SMILES: CC1(C)c2ccccc2-c2ccc(N(c3cccc(-c4ccccc4)c3)c3ccc4oc5cc(C6(C)c7ccccc7-c7ccccc76)ccc5c4c3)cc21 ; CC1(C)c2ccccc2-c2ccc(N(c3ccccc3)c3ccc4oc5cc(C6(C)c7ccccc7-c7ccccc76)ccc5c4c3)cc21 ; CC1(c2ccc3c(c2)oc2ccc(N(c4ccc(-c5ccccc5)cc4)c4ccc5ccccc5c4)cc23)c2ccccc2-c2ccccc21 ; CC1(c2ccc3c(c2)oc2ccc(N(c4ccc(-c5ccccc5)cc4)c4cccc5ccccc45)cc23)c2ccccc2-c2ccccc21]

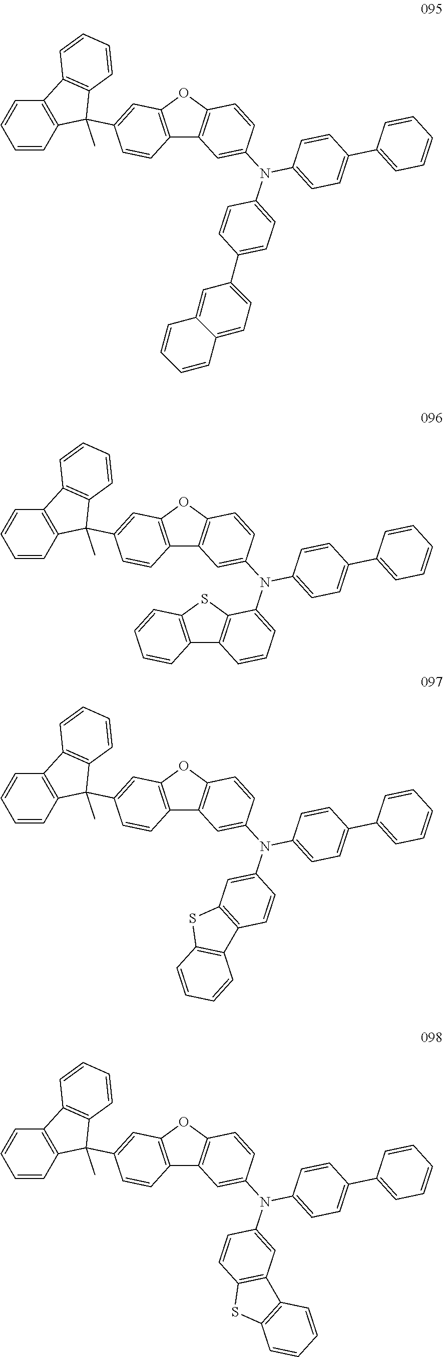 [SMILES: CC1(c2ccc3c(c2)oc2ccc(N(c4ccc(-c5ccccc5)cc4)c4ccc(-c5ccc6ccccc6c5)cc4)cc23)c2ccccc2-c2ccccc21 ; CC1(c2ccc3c(c2)oc2ccc(N(c4ccc(-c5ccccc5)cc4)c4cccc5c4sc4ccccc45)cc23)c2ccccc2-c2ccccc21 ; CC1(c2ccc3c(c2)oc2ccc(N(c4ccc(-c5ccccc5)cc4)c4ccc5c(c4)sc4ccccc45)cc23)c2ccccc2-c2ccccc21 ; CC1(c2ccc3c(c2)oc2ccc(N(c4ccc(-c5ccccc5)cc4)c4ccc5sc6ccccc6c5c4)cc23)c2ccccc2-c2ccccc21]

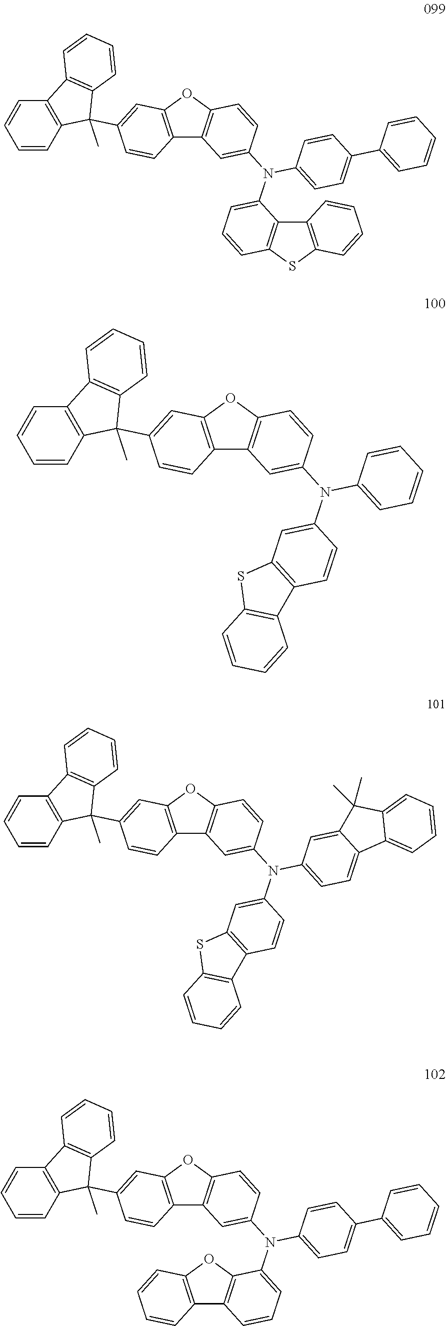 [SMILES: CC1(c2ccc3c(c2)oc2ccc(N(c4ccc(-c5ccccc5)cc4)c4cccc5sc6ccccc6c45)cc23)c2ccccc2-c2ccccc21 ; CC1(c2ccc3c(c2)oc2ccc(N(c4ccccc4)c4ccc5c(c4)sc4ccccc45)cc23)c2ccccc2-c2ccccc21 ; CC1(C)c2ccccc2-c2ccc(N(c3ccc4c(c3)sc3ccccc34)c3ccc4oc5cc(C6(C)c7ccccc7-c7ccccc76)ccc5c4c3)cc21 ; CC1(c2ccc3c(c2)oc2ccc(N(c4ccc(-c5ccccc5)cc4)c4cccc5c4oc4ccccc45)cc23)c2ccccc2-c2ccccc21]

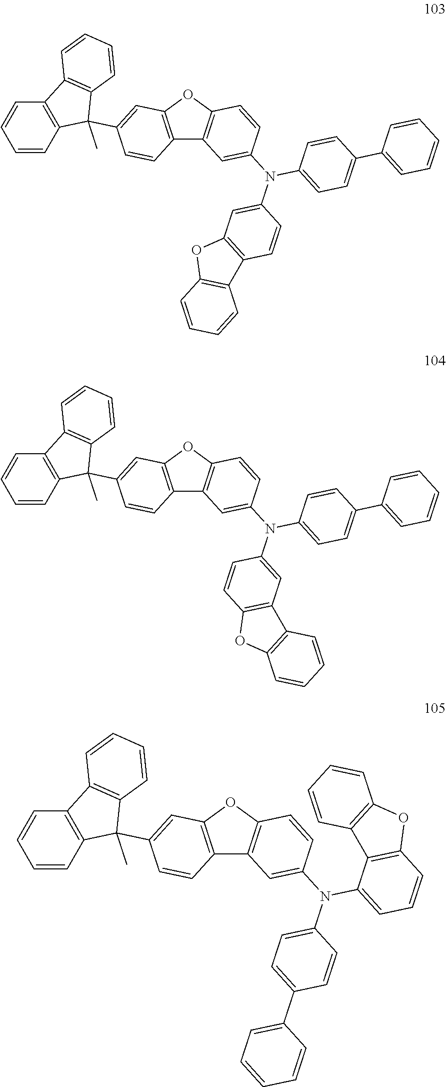 [SMILES: CC1(c2ccc3c(c2)oc2ccc(N(c4ccc(-c5ccccc5)cc4)c4ccc5c(c4)oc4ccccc45)cc23)c2ccccc2-c2ccccc21 ; CC1(c2ccc3c(c2)oc2ccc(N(c4ccc(-c5ccccc5)cc4)c4ccc5oc6ccccc6c5c4)cc23)c2ccccc2-c2ccccc21 ; CC1(c2ccc3c(c2)oc2ccc(N(c4ccc(-c5ccccc5)cc4)c4cccc5oc6ccccc6c45)cc23)c2ccccc2-c2ccccc21]

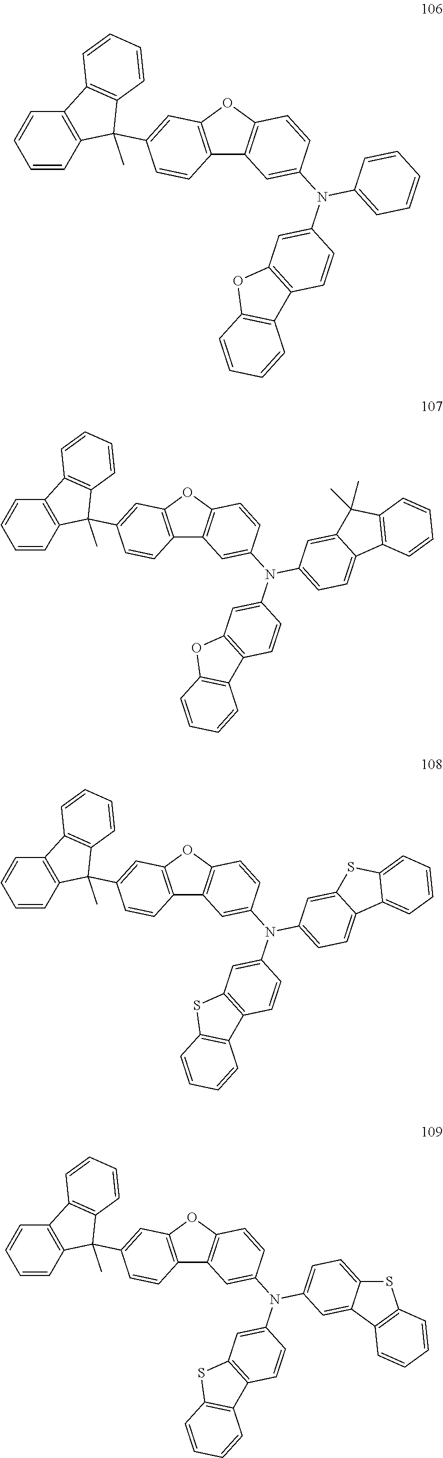 [SMILES: CC1(c2ccc3c(c2)oc2ccc(N(c4ccccc4)c4ccc5c(c4)oc4ccccc45)cc23)c2ccccc2-c2ccccc21 ; CC1(C)c2ccccc2-c2ccc(N(c3ccc4c(c3)oc3ccccc34)c3ccc4oc5cc(C6(C)c7ccccc7-c7ccccc76)ccc5c4c3)cc21 ; CC1(c2ccc3c(c2)oc2ccc(N(c4ccc5c(c4)sc4ccccc45)c4ccc5c(c4)sc4ccccc45)cc23)c2ccccc2-c2ccccc21 ; CC1(c2ccc3c(c2)oc2ccc(N(c4ccc5c(c4)sc4ccccc45)c4ccc5sc6ccccc6c5c4)cc23)c2ccccc2-c2ccccc21]

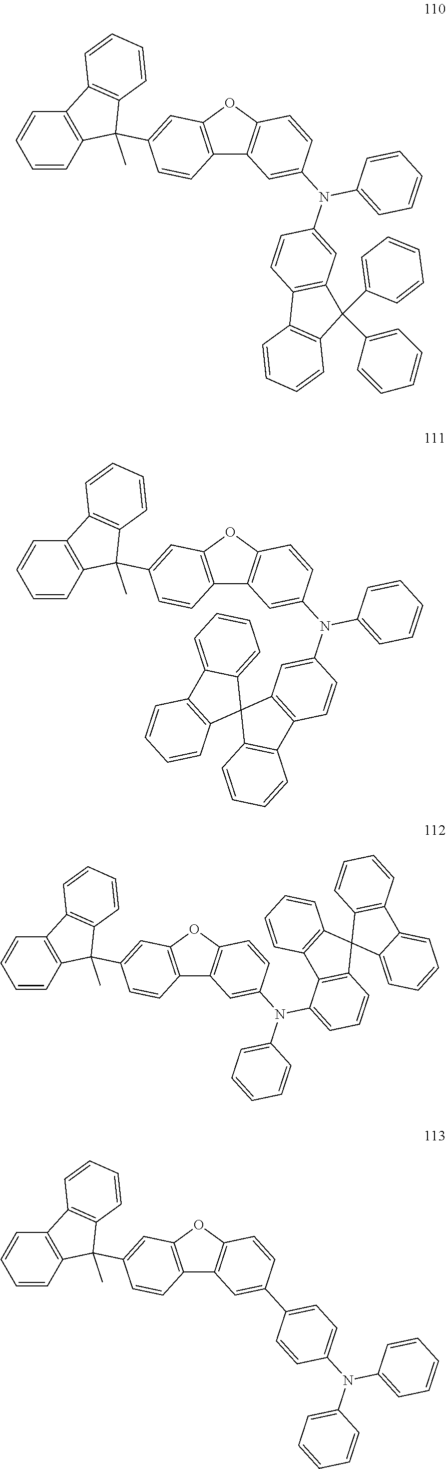 [SMILES: CC1(c2ccc3c(c2)oc2ccc(N(c4ccccc4)c4ccc5c(c4)C(c4ccccc4)(c4ccccc4)c4ccccc4-5)cc23)c2ccccc2-c2ccccc21 ; CC1(c2ccc3c(c2)oc2ccc(N(c4ccccc4)c4ccc5c(c4)C4(c6ccccc6-c6ccccc64)c4ccccc4-5)cc23)c2ccccc2-c2ccccc21 ; CC1(c2ccc3c(c2)oc2ccc(N(c4ccccc4)c4cccc5c4-c4ccccc4C54c5ccccc5-c5ccccc54)cc23)c2ccccc2-c2ccccc21 ; CC1(c2ccc3c(c2)oc2ccc(-c4ccc(N(c5ccccc5)c5ccccc5)cc4)cc23)c2ccccc2-c2ccccc21]

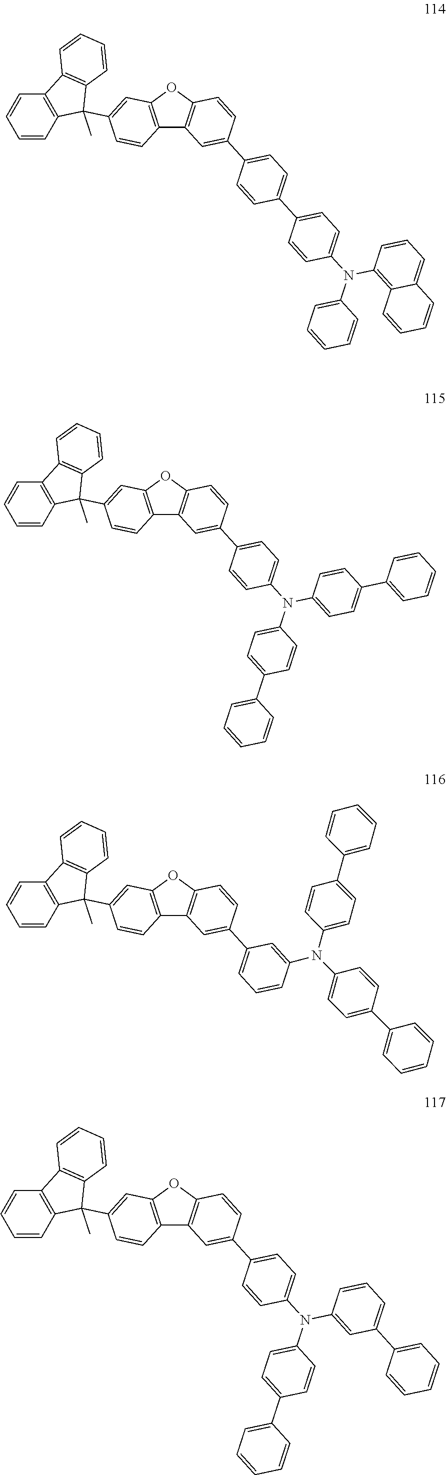 [SMILES: CC1(c2ccc3c(c2)oc2ccc(-c4ccc(-c5ccc(N(c6ccccc6)c6cccc7ccccc67)cc5)cc4)cc23)c2ccccc2-c2ccccc21 ; CC1(c2ccc3c(c2)oc2ccc(-c4ccc(N(c5ccc(-c6ccccc6)cc5)c5ccc(-c6ccccc6)cc5)cc4)cc23)c2ccccc2-c2ccccc21 ; CC1(c2ccc3c(c2)oc2ccc(-c4cccc(N(c5ccc(-c6ccccc6)cc5)c5ccc(-c6ccccc6)cc5)c4)cc23)c2ccccc2-c2ccccc21 ; CC1(c2ccc3c(c2)oc2ccc(-c4ccc(N(c5ccc(-c6ccccc6)cc5)c5cccc(-c6ccccc6)c5)cc4)cc23)c2ccccc2-c2ccccc21]

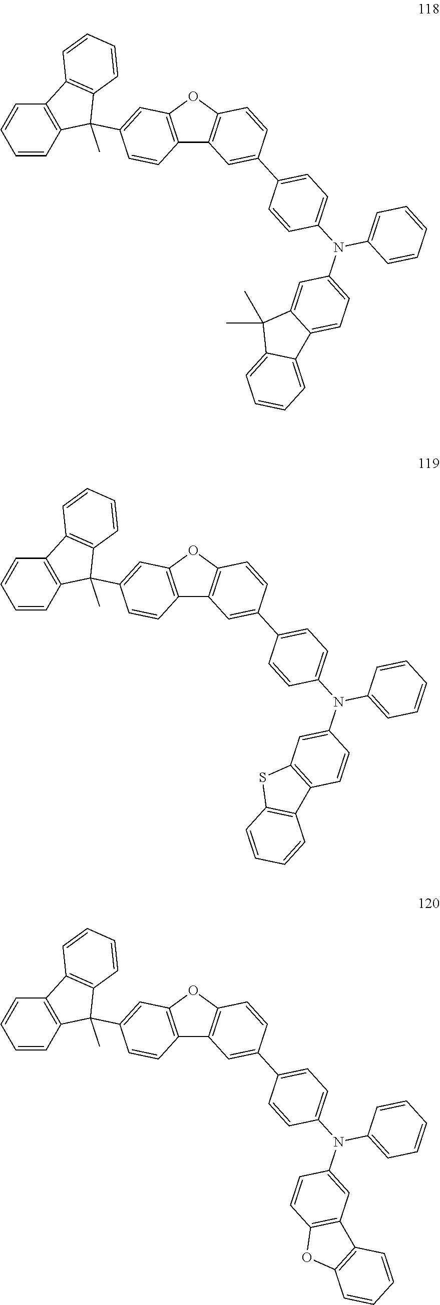 [SMILES: CC1(C)c2ccccc2-c2ccc(N(c3ccccc3)c3ccc(-c4ccc5oc6cc(C7(C)c8ccccc8-c8ccccc87)ccc6c5c4)cc3)cc21 ; CC1(c2ccc3c(c2)oc2ccc(-c4ccc(N(c5ccccc5)c5ccc6c(c5)sc5ccccc56)cc4)cc23)c2ccccc2-c2ccccc21 ; CC1(c2ccc3c(c2)oc2ccc(-c4ccc(N(c5ccccc5)c5ccc6oc7ccccc7c6c5)cc4)cc23)c2ccccc2-c2ccccc21]

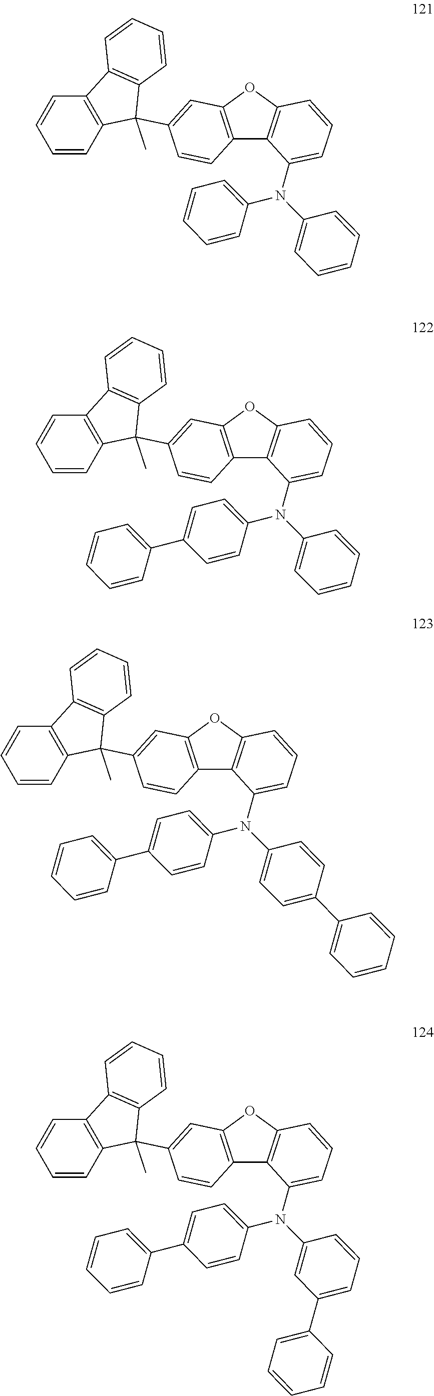 [SMILES: CC1(c2ccc3c(c2)oc2cccc(N(c4ccccc4)c4ccccc4)c23)c2ccccc2-c2ccccc21 ; CC1(c2ccc3c(c2)oc2cccc(N(c4ccccc4)c4ccc(-c5ccccc5)cc4)c23)c2ccccc2-c2ccccc21 ; CC1(c2ccc3c(c2)oc2cccc(N(c4ccc(-c5ccccc5)cc4)c4ccc(-c5ccccc5)cc4)c23)c2ccccc2-c2ccccc21 ; CC1(c2ccc3c(c2)oc2cccc(N(c4ccc(-c5ccccc5)cc4)c4cccc(-c5ccccc5)c4)c23)c2ccccc2-c2ccccc21]

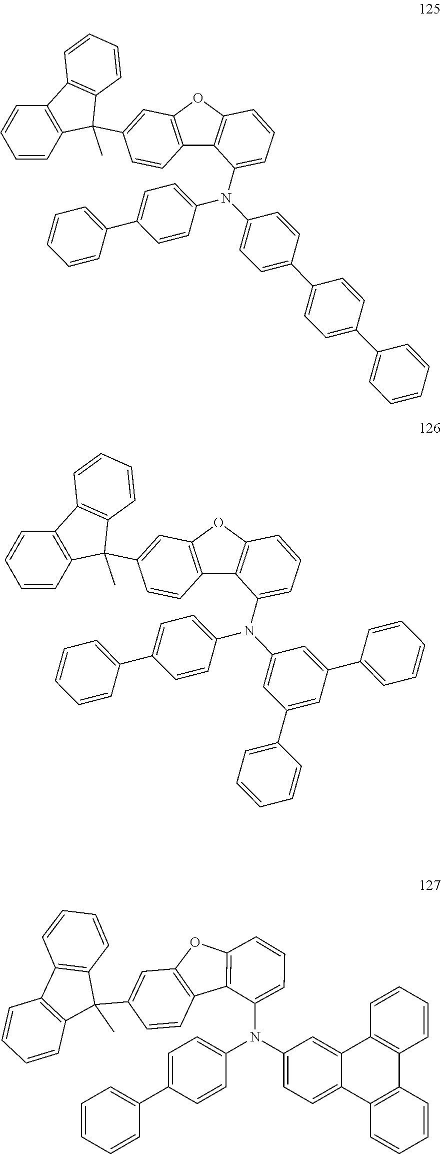 [SMILES: CC1(c2ccc3c(c2)oc2cccc(N(c4ccc(-c5ccccc5)cc4)c4ccc(-c5ccc(-c6ccccc6)cc5)cc4)c23)c2ccccc2-c2ccccc21 ; CC1(c2ccc3c(c2)oc2cccc(N(c4ccc(-c5ccccc5)cc4)c4cc(-c5ccccc5)cc(-c5ccccc5)c4)c23)c2ccccc2-c2ccccc21 ; CC1(c2ccc3c(c2)oc2cccc(N(c4ccc(-c5ccccc5)cc4)c4ccc5c6ccccc6c6ccccc6c5c4)c23)c2ccccc2-c2ccccc21]

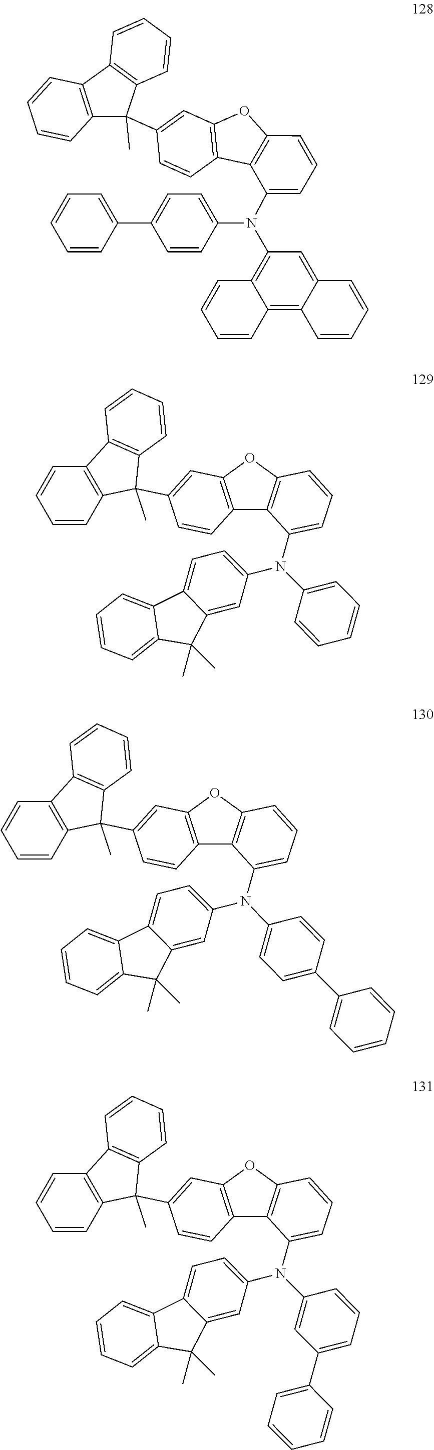 [SMILES: CC1(c2ccc3c(c2)oc2cccc(N(c4ccc(-c5ccccc5)cc4)c4cc5ccccc5c5ccccc45)c23)c2ccccc2-c2ccccc21 ; CC1(C)c2ccccc2-c2ccc(N(c3ccccc3)c3cccc4oc5cc(C6(C)c7ccccc7-c7ccccc76)ccc5c34)cc21 ; CC1(C)c2ccccc2-c2ccc(N(c3ccc(-c4ccccc4)cc3)c3cccc4oc5cc(C6(C)c7ccccc7-c7ccccc76)ccc5c34)cc21 ; CC1(C)c2ccccc2-c2ccc(N(c3cccc(-c4ccccc4)c3)c3cccc4oc5cc(C6(C)c7ccccc7-c7ccccc76)ccc5c34)cc21]

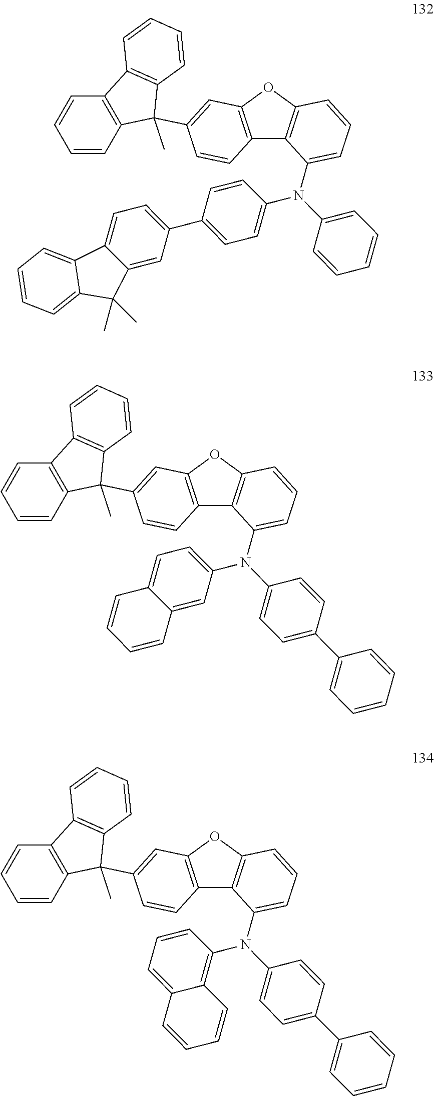 [SMILES: CC1(C)c2ccccc2-c2ccc(-c3ccc(N(c4ccccc4)c4cccc5oc6cc(C7(C)c8ccccc8-c8ccccc87)ccc6c45)cc3)cc21 ; CC1(c2ccc3c(c2)oc2cccc(N(c4ccc(-c5ccccc5)cc4)c4ccc5ccccc5c4)c23)c2ccccc2-c2ccccc21 ; CC1(c2ccc3c(c2)oc2cccc(N(c4ccc(-c5ccccc5)cc4)c4cccc5ccccc45)c23)c2ccccc2-c2ccccc21]

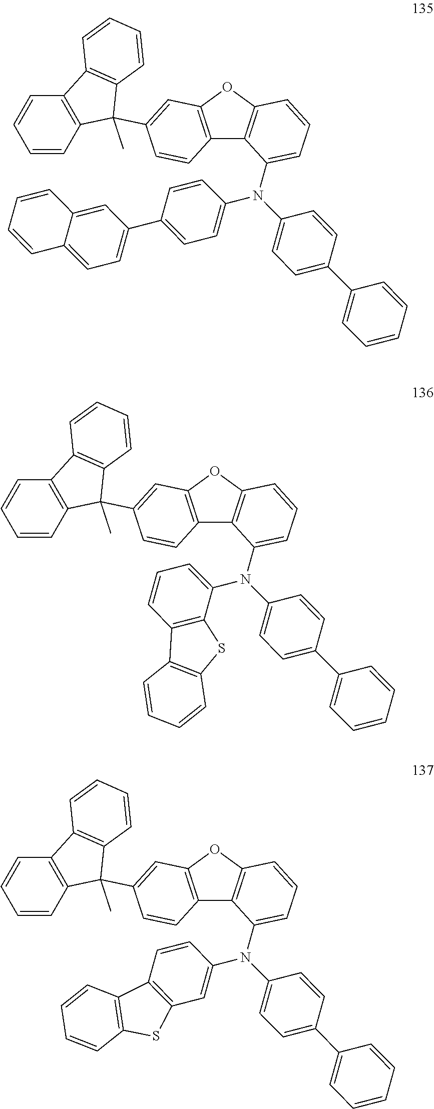 [SMILES: CC1(c2ccc3c(c2)oc2cccc(N(c4ccc(-c5ccccc5)cc4)c4ccc(-c5ccc6ccccc6c5)cc4)c23)c2ccccc2-c2ccccc21 ; CC1(c2ccc3c(c2)oc2cccc(N(c4ccc(-c5ccccc5)cc4)c4cccc5c4sc4ccccc45)c23)c2ccccc2-c2ccccc21 ; CC1(c2ccc3c(c2)oc2cccc(N(c4ccc(-c5ccccc5)cc4)c4ccc5c(c4)sc4ccccc45)c23)c2ccccc2-c2ccccc21]

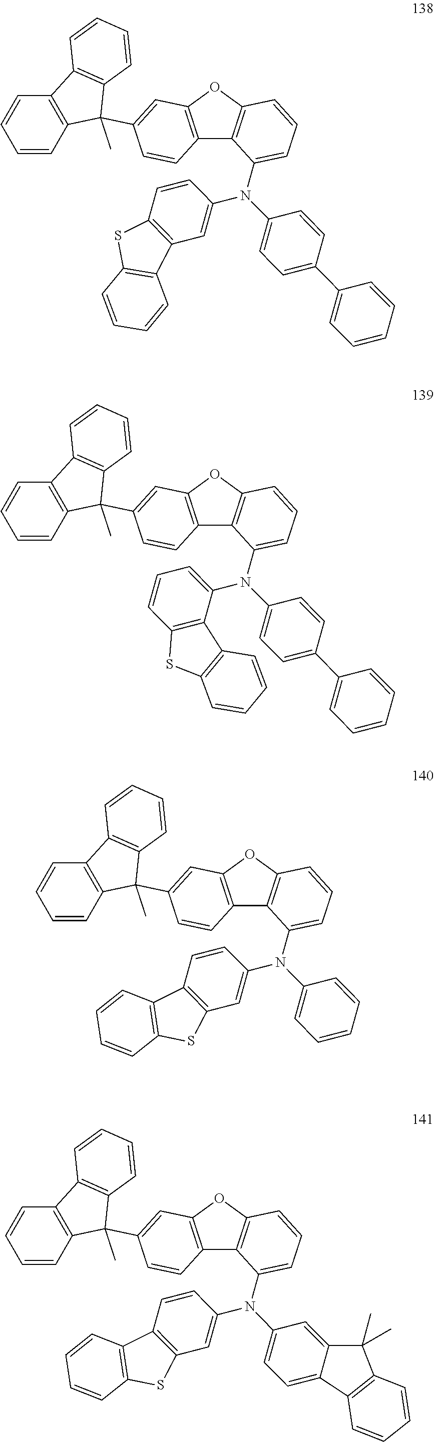 [SMILES: CC1(c2ccc3c(c2)oc2cccc(N(c4ccc(-c5ccccc5)cc4)c4ccc5sc6ccccc6c5c4)c23)c2ccccc2-c2ccccc21 ; CC1(c2ccc3c(c2)oc2cccc(N(c4ccc(-c5ccccc5)cc4)c4cccc5sc6ccccc6c45)c23)c2ccccc2-c2ccccc21 ; CC1(c2ccc3c(c2)oc2cccc(N(c4ccccc4)c4ccc5c(c4)sc4ccccc45)c23)c2ccccc2-c2ccccc21 ; CC1(C)c2ccccc2-c2ccc(N(c3ccc4c(c3)sc3ccccc34)c3cccc4oc5cc(C6(C)c7ccccc7-c7ccccc76)ccc5c34)cc21]

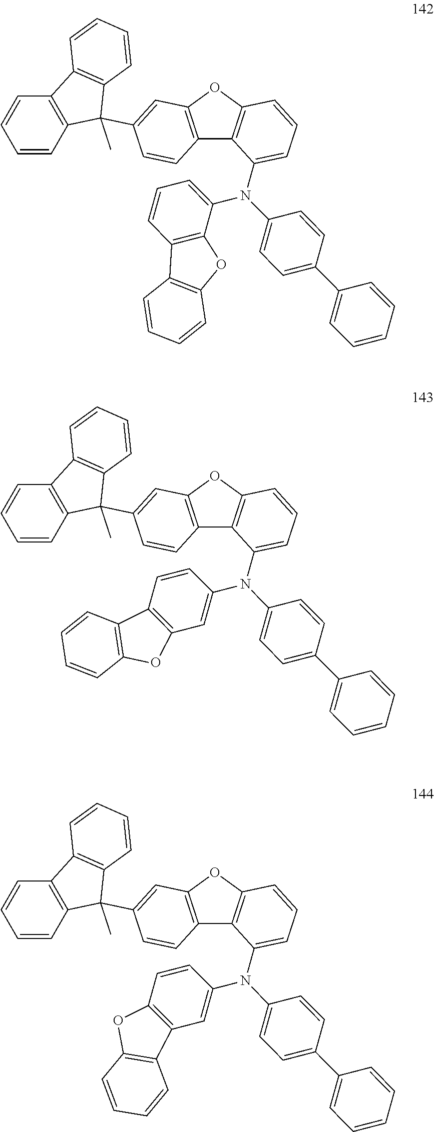 [SMILES: CC1(c2ccc3c(c2)oc2cccc(N(c4ccc(-c5ccccc5)cc4)c4cccc5c4oc4ccccc45)c23)c2ccccc2-c2ccccc21 ; CC1(c2ccc3c(c2)oc2cccc(N(c4ccc(-c5ccccc5)cc4)c4ccc5c(c4)oc4ccccc45)c23)c2ccccc2-c2ccccc21 ; CC1(c2ccc3c(c2)oc2cccc(N(c4ccc(-c5ccccc5)cc4)c4ccc5oc6ccccc6c5c4)c23)c2ccccc2-c2ccccc21]

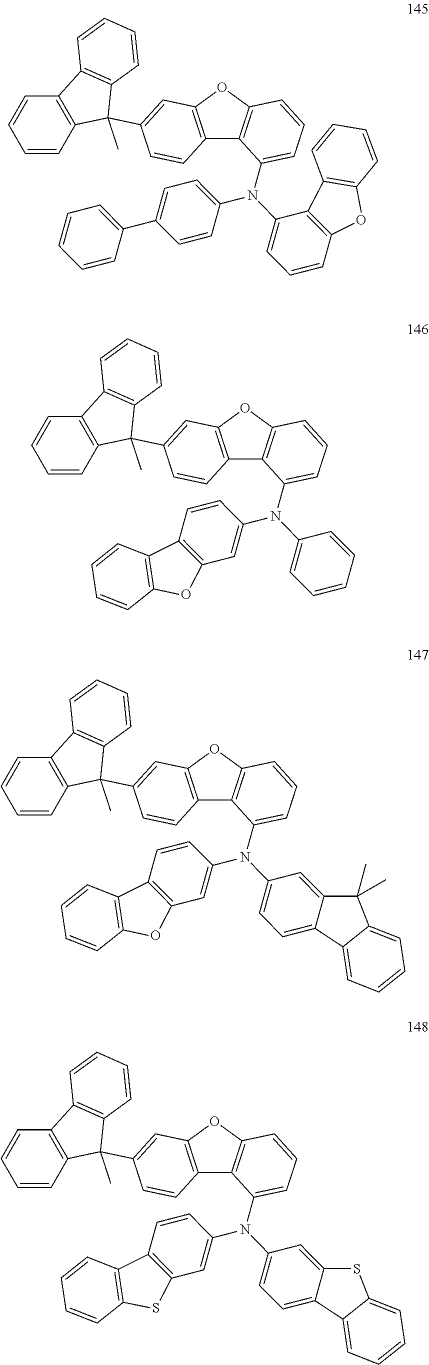 [SMILES: CC1(c2ccc3c(c2)oc2cccc(N(c4ccc(-c5ccccc5)cc4)c4cccc5oc6ccccc6c45)c23)c2ccccc2-c2ccccc21 ; CC1(c2ccc3c(c2)oc2cccc(N(c4ccccc4)c4ccc5c(c4)oc4ccccc45)c23)c2ccccc2-c2ccccc21 ; CC1(C)c2ccccc2-c2ccc(N(c3ccc4c(c3)oc3ccccc34)c3cccc4oc5cc(C6(C)c7ccccc7-c7ccccc76)ccc5c34)cc21 ; CC1(c2ccc3c(c2)oc2cccc(N(c4ccc5c(c4)sc4ccccc45)c4ccc5c(c4)sc4ccccc45)c23)c2ccccc2-c2ccccc21]

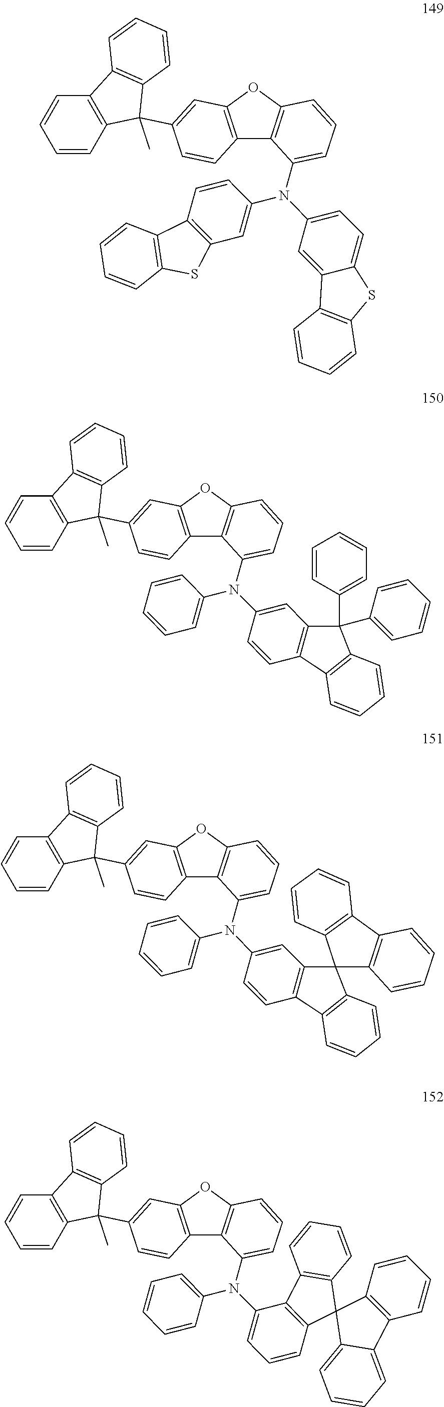 [SMILES: CC1(c2ccc3c(c2)oc2cccc(N(c4ccc5c(c4)sc4ccccc45)c4ccc5sc6ccccc6c5c4)c23)c2ccccc2-c2ccccc21 ; CC1(c2ccc3c(c2)oc2cccc(N(c4ccccc4)c4ccc5c(c4)C(c4ccccc4)(c4ccccc4)c4ccccc4-5)c23)c2ccccc2-c2ccccc21 ; CC1(c2ccc3c(c2)oc2cccc(N(c4ccccc4)c4ccc5c(c4)C4(c6ccccc6-c6ccccc64)c4ccccc4-5)c23)c2ccccc2-c2ccccc21 ; CC1(c2ccc3c(c2)oc2cccc(N(c4ccccc4)c4cccc5c4-c4ccccc4C54c5ccccc5-c5ccccc54)c23)c2ccccc2-c2ccccc21]

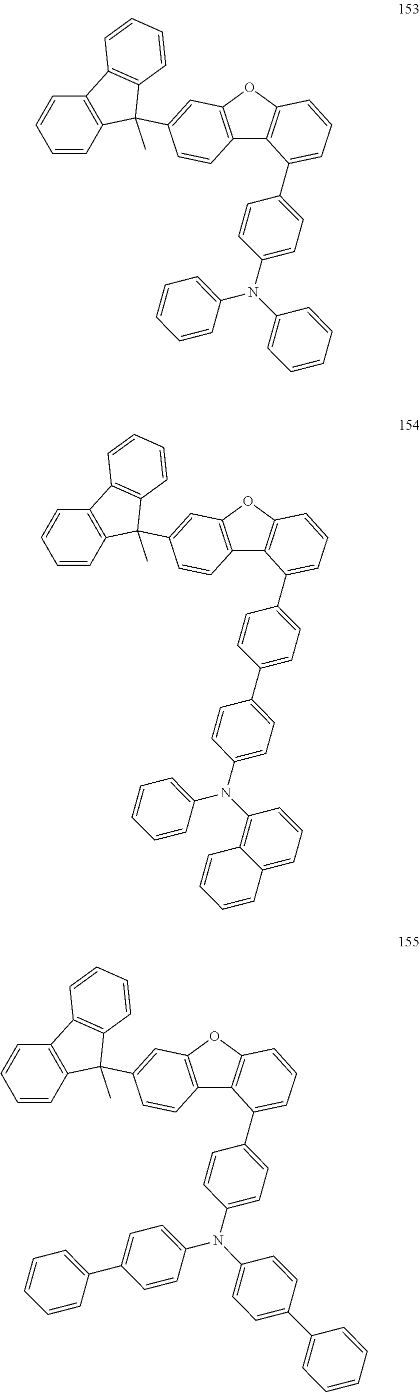 [SMILES: CC1(c2ccc3c(c2)oc2cccc(-c4ccc(N(c5ccccc5)c5ccccc5)cc4)c23)c2ccccc2-c2ccccc21 ; CC1(c2ccc3c(c2)oc2cccc(-c4ccc(-c5ccc(N(c6ccccc6)c6cccc7ccccc67)cc5)cc4)c23)c2ccccc2-c2ccccc21 ; CC1(c2ccc3c(c2)oc2cccc(-c4ccc(N(c5ccc(-c6ccccc6)cc5)c5ccc(-c6ccccc6)cc5)cc4)c23)c2ccccc2-c2ccccc21]

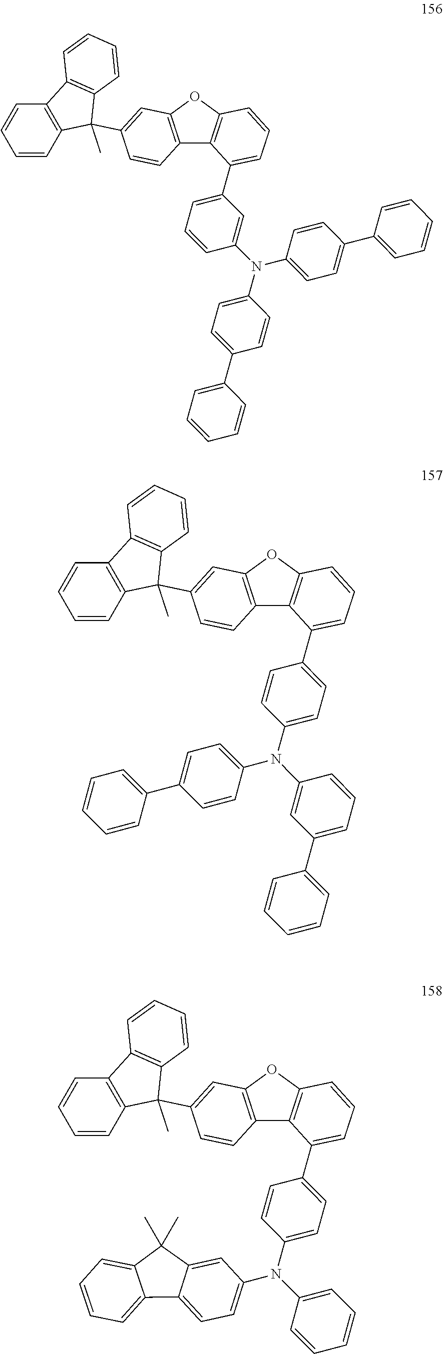 [SMILES: CC1(c2ccc3c(c2)oc2cccc(-c4cccc(N(c5ccc(-c6ccccc6)cc5)c5ccc(-c6ccccc6)cc5)c4)c23)c2ccccc2-c2ccccc21 ; CC1(c2ccc3c(c2)oc2cccc(-c4ccc(N(c5ccc(-c6ccccc6)cc5)c5cccc(-c6ccccc6)c5)cc4)c23)c2ccccc2-c2ccccc21 ; CC1(C)c2ccccc2-c2ccc(N(c3ccccc3)c3ccc(-c4cccc5oc6cc(C7(C)c8ccccc8-c8ccccc87)ccc6c45)cc3)cc21]

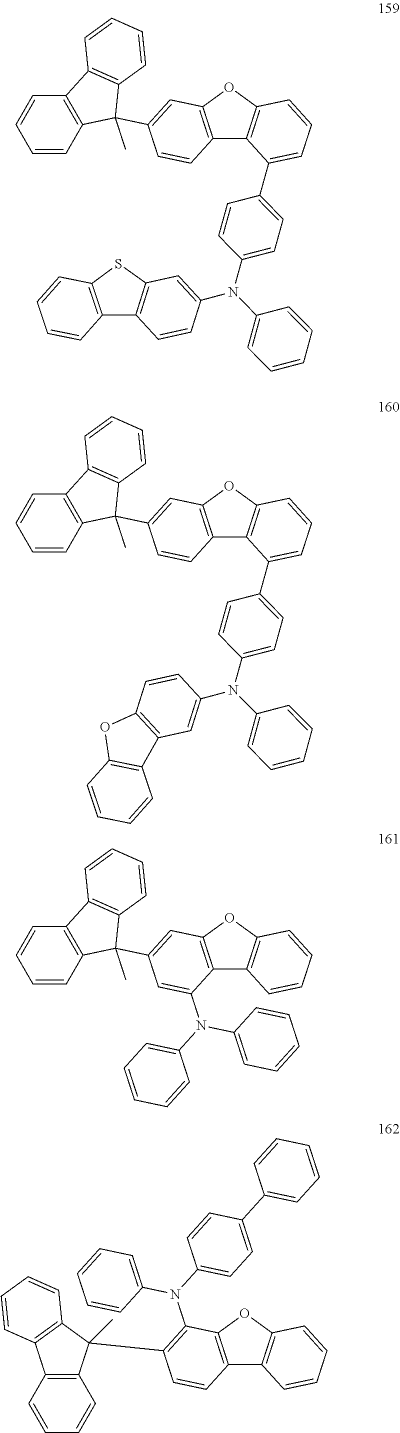

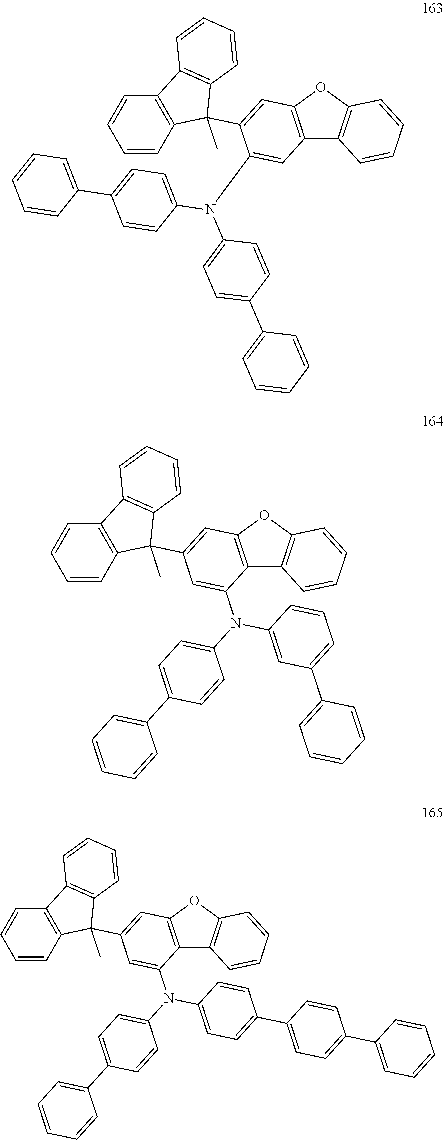 [SMILES: CC1(c2cc3oc4ccccc4c3cc2N(c2ccc(-c3ccccc3)cc2)c2ccc(-c3ccccc3)cc2)c2ccccc2-c2ccccc21 ; CC1(c2cc(N(c3ccc(-c4ccccc4)cc3)c3cccc(-c4ccccc4)c3)c3c(c2)oc2ccccc23)c2ccccc2-c2ccccc21 ; CC1(c2cc(N(c3ccc(-c4ccccc4)cc3)c3ccc(-c4ccc(-c5ccccc5)cc4)cc3)c3c(c2)oc2ccccc23)c2ccccc2-c2ccccc21]

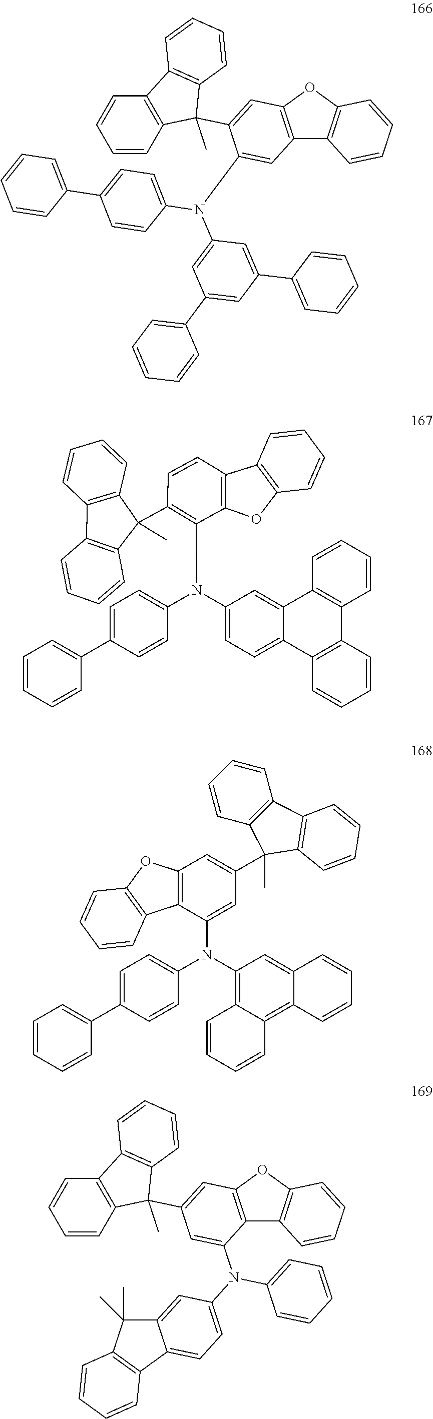 [SMILES: CC1(c2cc3oc4ccccc4c3cc2N(c2ccc(-c3ccccc3)cc2)c2cc(-c3ccccc3)cc(-c3ccccc3)c2)c2ccccc2-c2ccccc21 ; CC1(c2ccc3c(oc4ccccc43)c2N(c2ccc(-c3ccccc3)cc2)c2ccc3c4ccccc4c4ccccc4c3c2)c2ccccc2-c2ccccc21 ; CC1(c2cc(N(c3ccc(-c4ccccc4)cc3)c3cc4ccccc4c4ccccc34)c3c(c2)oc2ccccc23)c2ccccc2-c2ccccc21 ; CC1(C)c2ccccc2-c2ccc(N(c3ccccc3)c3cc(C4(C)c5ccccc5-c5ccccc54)cc4oc5ccccc5c34)cc21]

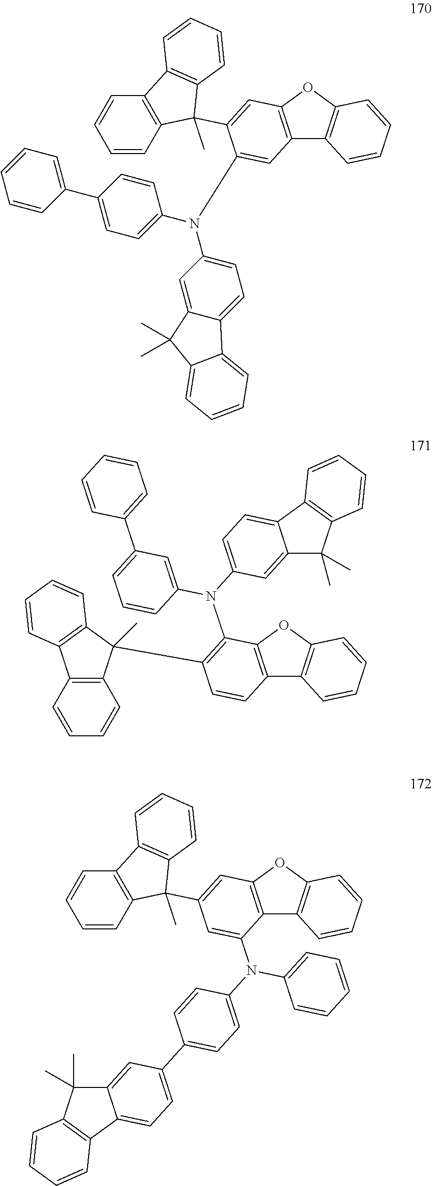 [SMILES: CC1(C)c2ccccc2-c2ccc(N(c3ccc(-c4ccccc4)cc3)c3cc4c(cc3C3(C)c5ccccc5-c5ccccc53)oc3ccccc34)cc21 ; CC1(C)c2ccccc2-c2ccc(N(c3cccc(-c4ccccc4)c3)c3c(C4(C)c5ccccc5-c5ccccc54)ccc4c3oc3ccccc34)cc21 ; CC1(C)c2ccccc2-c2ccc(-c3ccc(N(c4ccccc4)c4cc(C5(C)c6ccccc6-c6ccccc65)cc5oc6ccccc6c45)cc3)cc21]

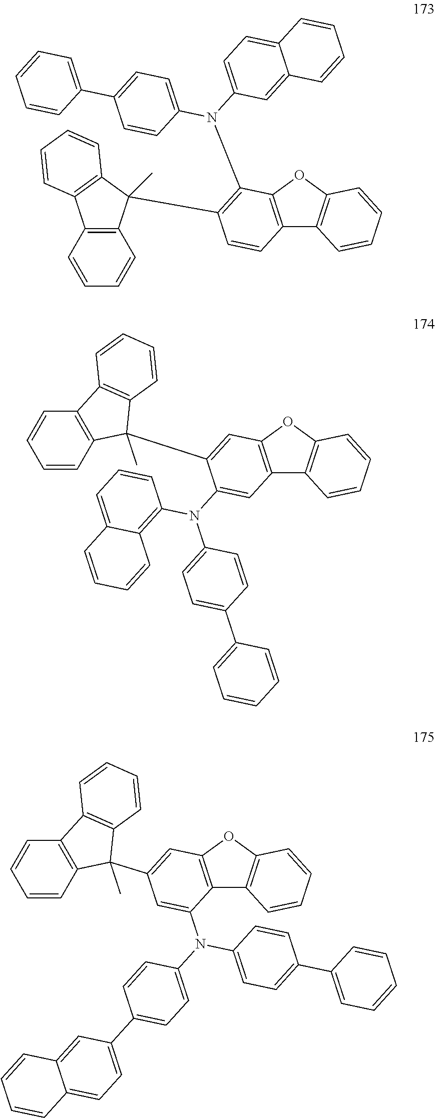 [SMILES: CC1(c2ccc3c(oc4ccccc43)c2N(c2ccc(-c3ccccc3)cc2)c2ccc3ccccc3c2)c2ccccc2-c2ccccc21 ; CC1(c2cc3oc4ccccc4c3cc2N(c2ccc(-c3ccccc3)cc2)c2cccc3ccccc23)c2ccccc2-c2ccccc21 ; CC1(c2cc(N(c3ccc(-c4ccccc4)cc3)c3ccc(-c4ccc5ccccc5c4)cc3)c3c(c2)oc2ccccc23)c2ccccc2-c2ccccc21]

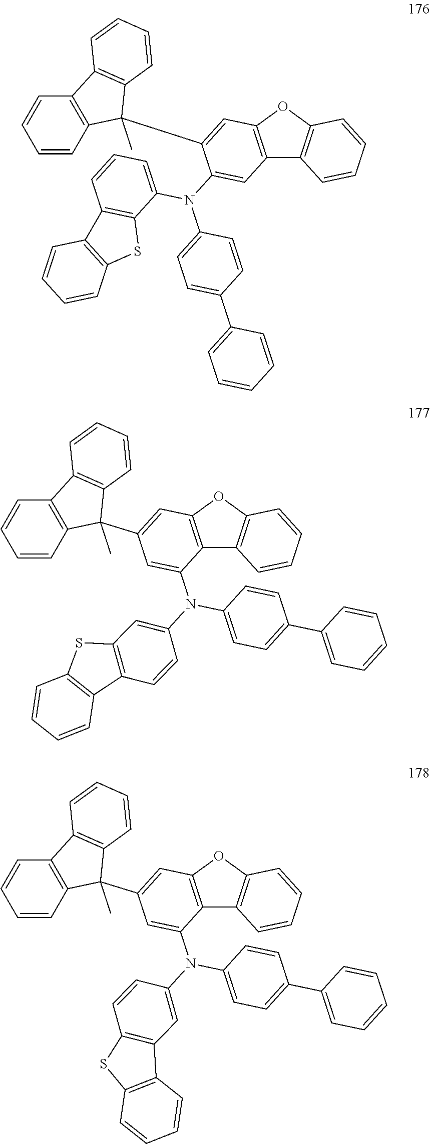 [SMILES: CC1(c2cc3oc4ccccc4c3cc2N(c2ccc(-c3ccccc3)cc2)c2cccc3c2sc2ccccc23)c2ccccc2-c2ccccc21 ; CC1(c2cc(N(c3ccc(-c4ccccc4)cc3)c3ccc4c(c3)sc3ccccc34)c3c(c2)oc2ccccc23)c2ccccc2-c2ccccc21 ; CC1(c2cc(N(c3ccc(-c4ccccc4)cc3)c3ccc4sc5ccccc5c4c3)c3c(c2)oc2ccccc23)c2ccccc2-c2ccccc21]

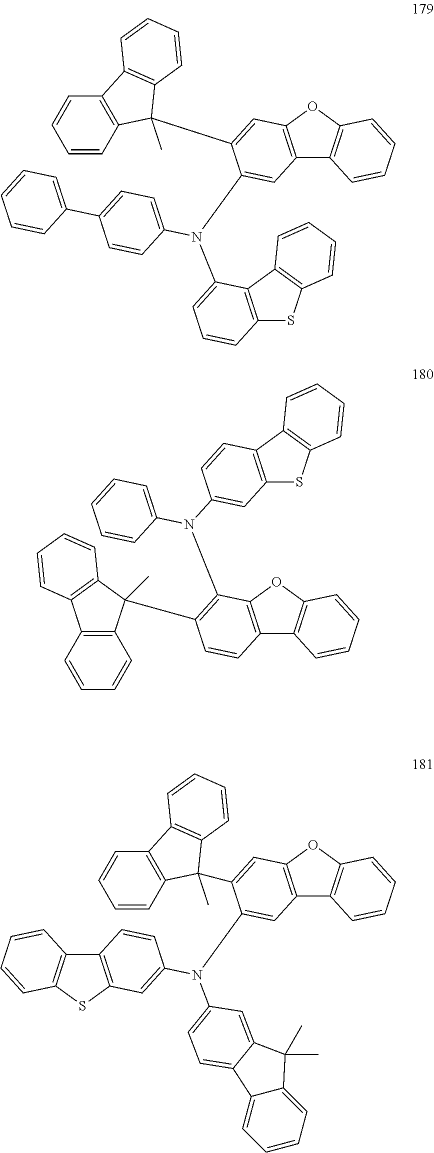 [SMILES: CC1(c2cc3oc4ccccc4c3cc2N(c2ccc(-c3ccccc3)cc2)c2cccc3sc4ccccc4c23)c2ccccc2-c2ccccc21 ; CC1(c2ccc3c(oc4ccccc43)c2N(c2ccccc2)c2ccc3c(c2)sc2ccccc23)c2ccccc2-c2ccccc21 ; CC1(C)c2ccccc2-c2ccc(N(c3ccc4c(c3)sc3ccccc34)c3cc4c(cc3C3(C)c5ccccc5-c5ccccc53)oc3ccccc34)cc21]

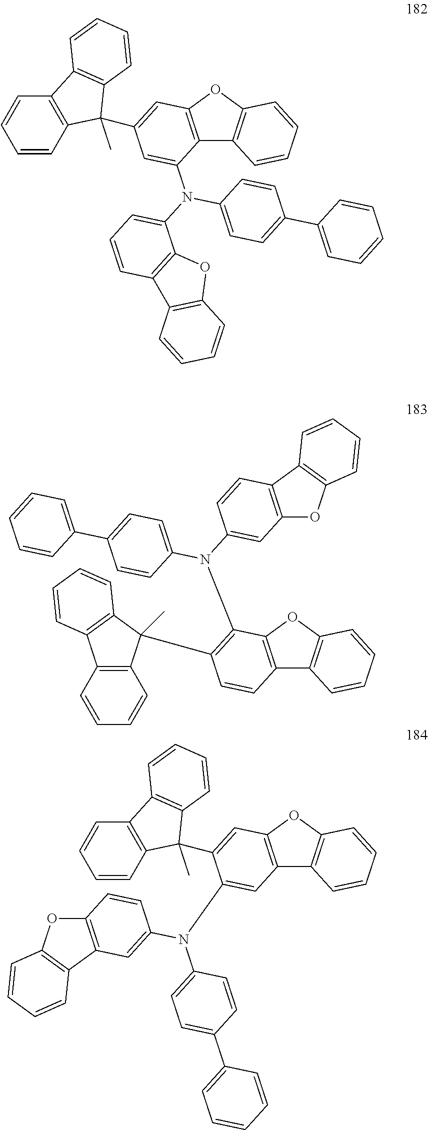

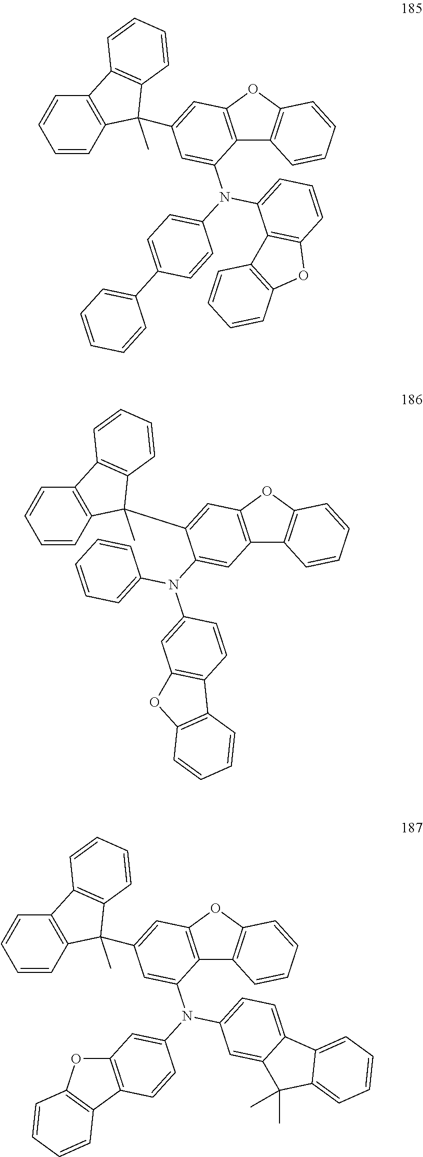 [SMILES: CC1(c2cc(N(c3ccc(-c4ccccc4)cc3)c3cccc4oc5ccccc5c34)c3c(c2)oc2ccccc23)c2ccccc2-c2ccccc21 ; CC1(c2cc3oc4ccccc4c3cc2N(c2ccccc2)c2ccc3c(c2)oc2ccccc23)c2ccccc2-c2ccccc21 ; CC1(C)c2ccccc2-c2ccc(N(c3ccc4c(c3)oc3ccccc34)c3cc(C4(C)c5ccccc5-c5ccccc54)cc4oc5ccccc5c34)cc21]

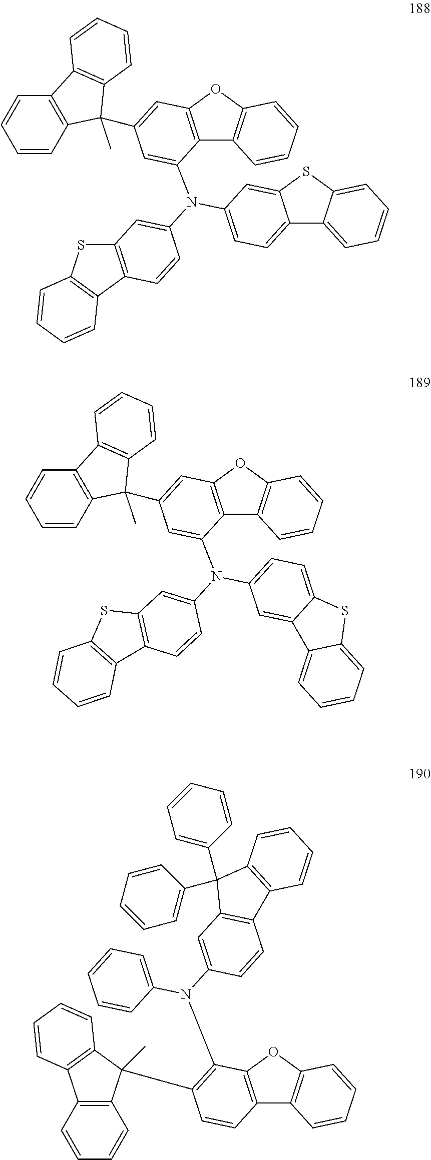 [SMILES: CC1(c2cc(N(c3ccc4c(c3)sc3ccccc34)c3ccc4c(c3)sc3ccccc34)c3c(c2)oc2ccccc23)c2ccccc2-c2ccccc21 ; CC1(c2cc(N(c3ccc4c(c3)sc3ccccc34)c3ccc4sc5ccccc5c4c3)c3c(c2)oc2ccccc23)c2ccccc2-c2ccccc21 ; CC1(c2ccc3c(oc4ccccc43)c2N(c2ccccc2)c2ccc3c(c2)C(c2ccccc2)(c2ccccc2)c2ccccc2-3)c2ccccc2-c2ccccc21]

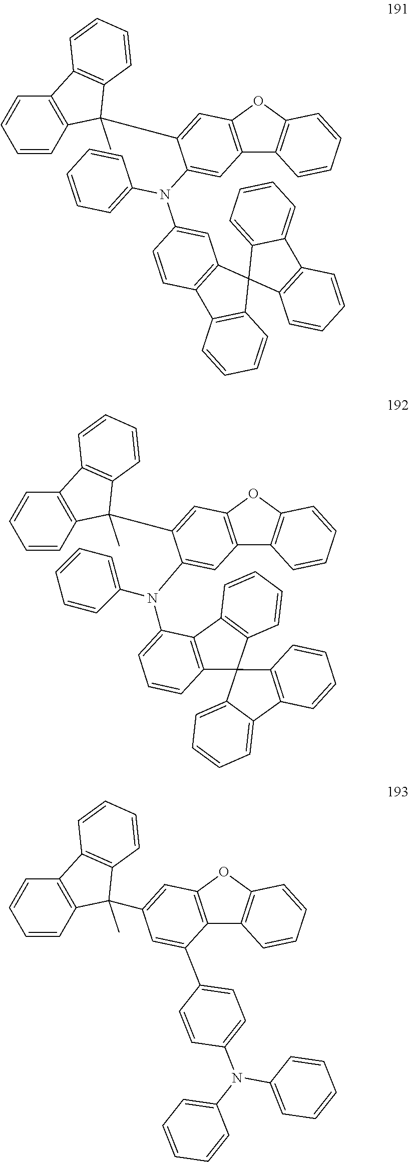 [SMILES: CC1(c2cc3oc4ccccc4c3cc2N(c2ccccc2)c2ccc3c(c2)C2(c4ccccc4-c4ccccc42)c2ccccc2-3)c2ccccc2-c2ccccc21 ; CC1(c2cc3oc4ccccc4c3cc2N(c2ccccc2)c2cccc3c2-c2ccccc2C32c3ccccc3-c3ccccc32)c2ccccc2-c2ccccc21 ; CC1(c2cc(-c3ccc(N(c4ccccc4)c4ccccc4)cc3)c3c(c2)oc2ccccc23)c2ccccc2-c2ccccc21]

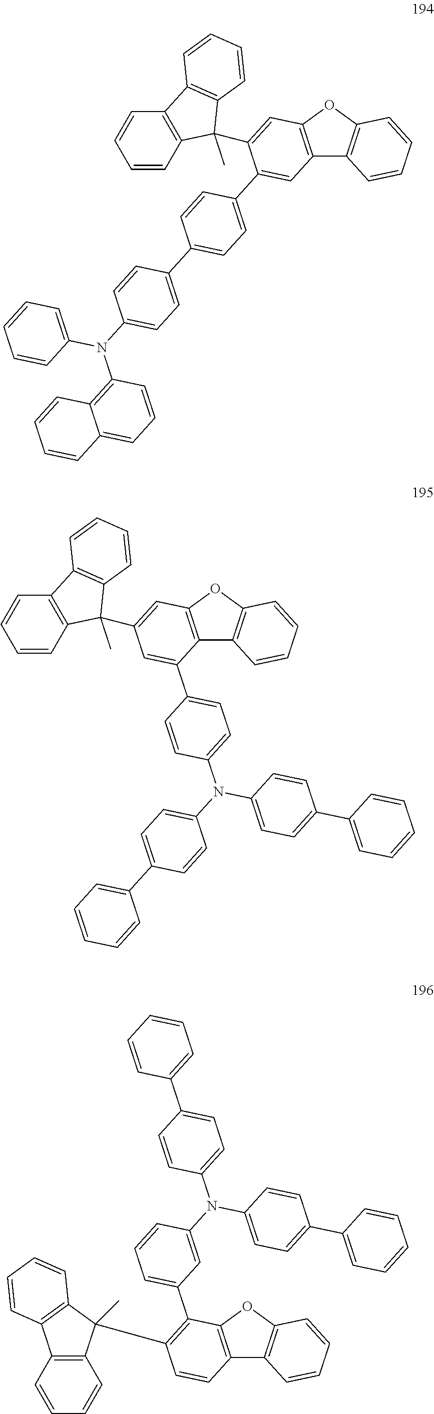 [SMILES: CC1(c2cc3oc4ccccc4c3cc2-c2ccc(-c3ccc(N(c4ccccc4)c4cccc5ccccc45)cc3)cc2)c2ccccc2-c2ccccc21 ; CC1(c2cc(-c3ccc(N(c4ccc(-c5ccccc5)cc4)c4ccc(-c5ccccc5)cc4)cc3)c3c(c2)oc2ccccc23)c2ccccc2-c2ccccc21 ; CC1(c2ccc3c(oc4ccccc43)c2-c2cccc(N(c3ccc(-c4ccccc4)cc3)c3ccc(-c4ccccc4)cc3)c2)c2ccccc2-c2ccccc21]

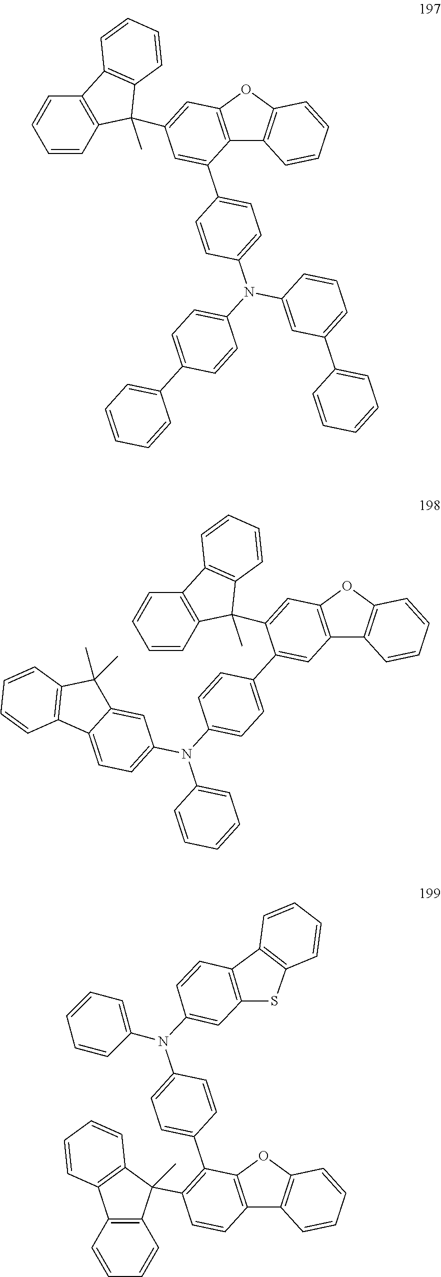 [SMILES: CC1(c2cc(-c3ccc(N(c4ccc(-c5ccccc5)cc4)c4cccc(-c5ccccc5)c4)cc3)c3c(c2)oc2ccccc23)c2ccccc2-c2ccccc21 ; CC1(C)c2ccccc2-c2ccc(N(c3ccccc3)c3ccc(-c4cc5c(cc4C4(C)c6ccccc6-c6ccccc64)oc4ccccc45)cc3)cc21 ; CC1(c2ccc3c(oc4ccccc43)c2-c2ccc(N(c3ccccc3)c3ccc4c(c3)sc3ccccc34)cc2)c2ccccc2-c2ccccc21]

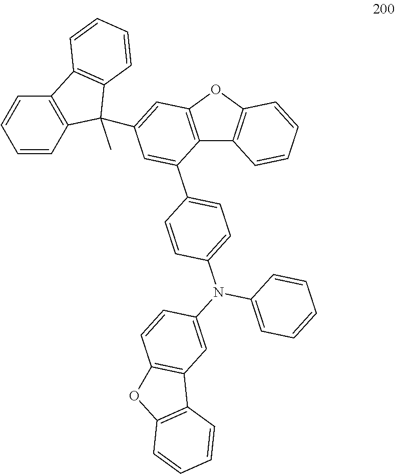 [SMILES: CC1(c2cc(-c3ccc(N(c4ccccc4)c4ccc5oc6ccccc6c5c4)cc3)c3c(c2)oc2ccccc23)c2ccccc2-c2ccccc21]

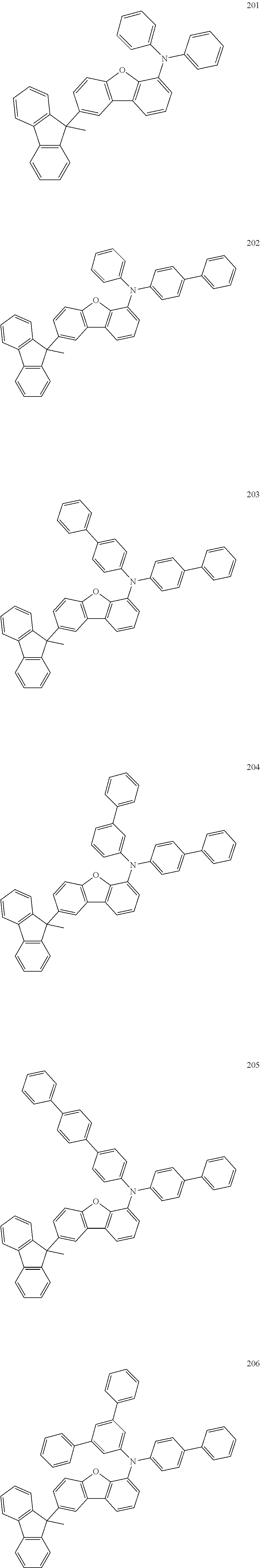

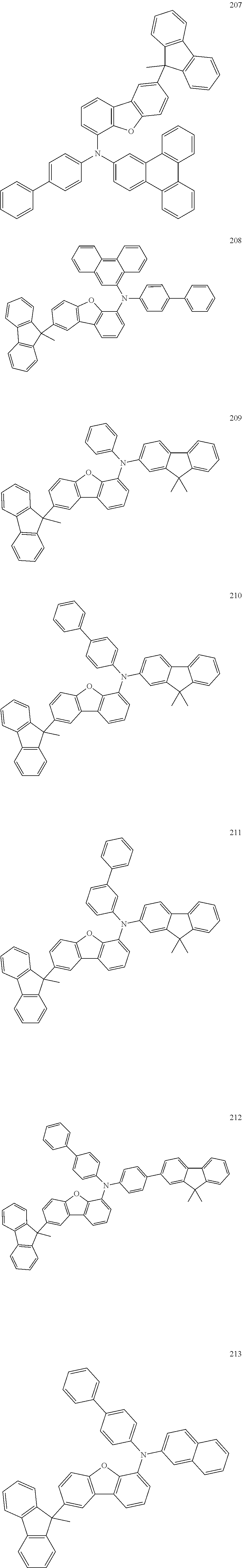

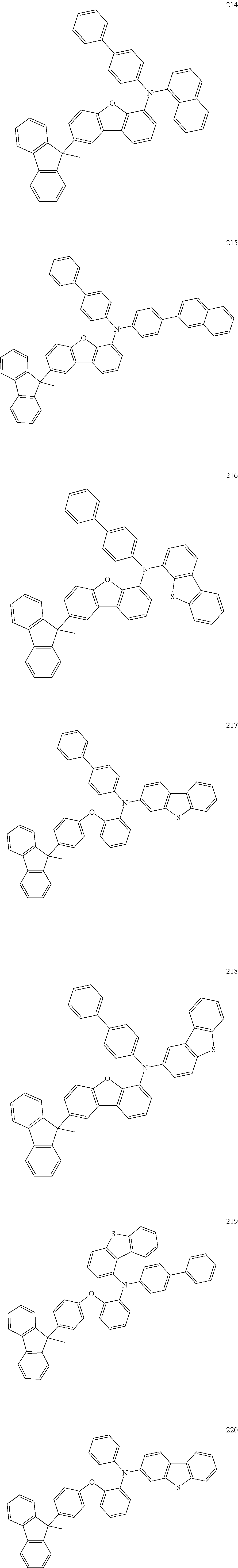

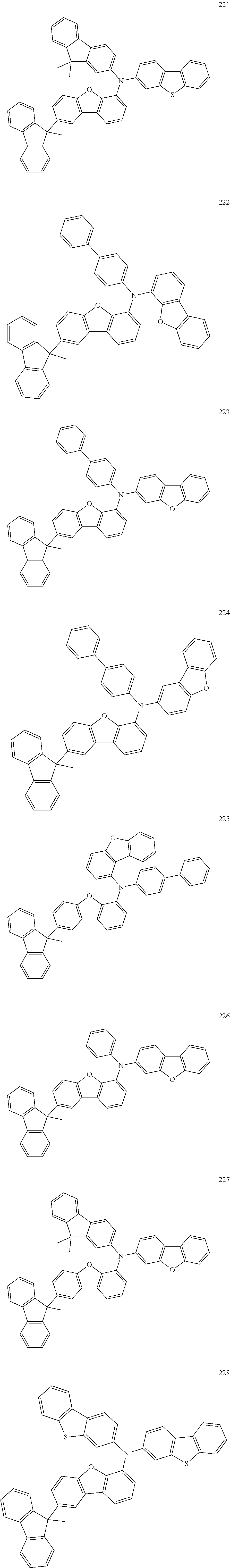

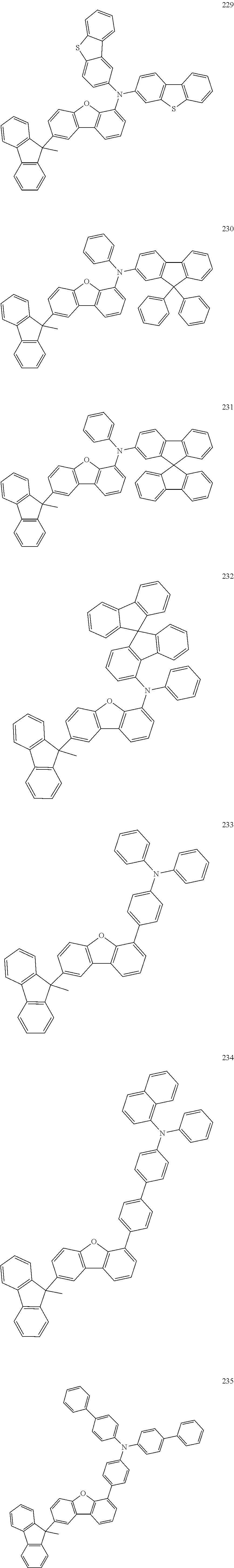

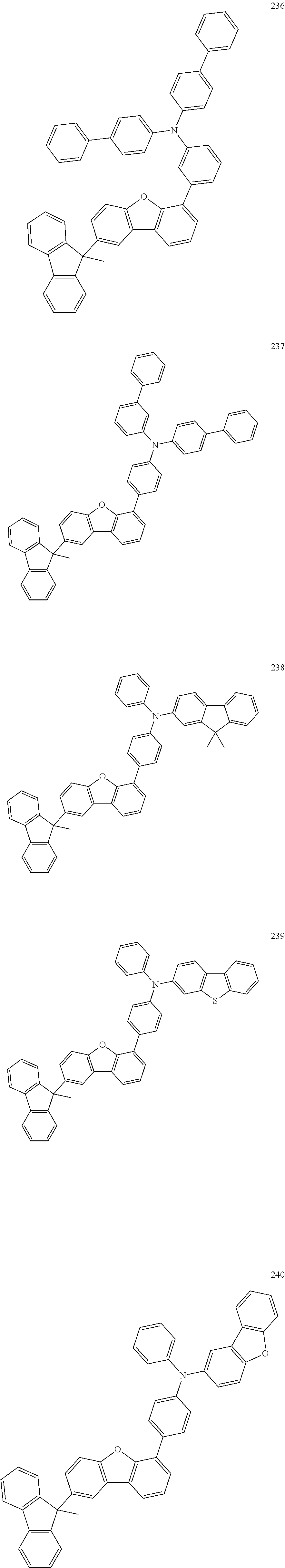 [SMILES: CC1(c2ccc3oc4c(-c5cccc(N(c6ccc(-c7ccccc7)cc6)c6ccc(-c7ccccc7)cc6)c5)cccc4c3c2)c2ccccc2-c2ccccc21 ; CC1(c2ccc3oc4c(-c5ccc(N(c6ccc(-c7ccccc7)cc6)c6cccc(-c7ccccc7)c6)cc5)cccc4c3c2)c2ccccc2-c2ccccc21 ; CC1(C)c2ccccc2-c2ccc(N(c3ccccc3)c3ccc(-c4cccc5c4oc4ccc(C6(C)c7ccccc7-c7ccccc76)cc45)cc3)cc21 ; CC1(c2ccc3oc4c(-c5ccc(N(c6ccccc6)c6ccc7c(c6)sc6ccccc67)cc5)cccc4c3c2)c2ccccc2-c2ccccc21 ; CC1(c2ccc3oc4c(-c5ccc(N(c6ccccc6)c6ccc7oc8ccccc8c7c6)cc5)cccc4c3c2)c2ccccc2-c2ccccc21]

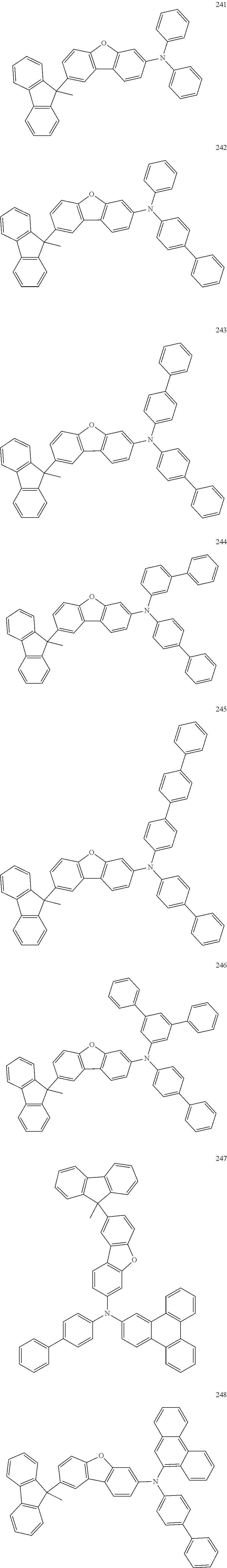

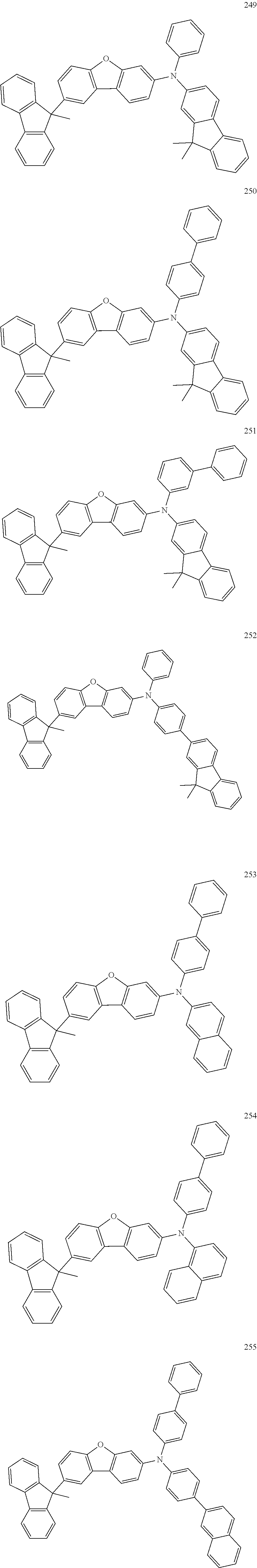

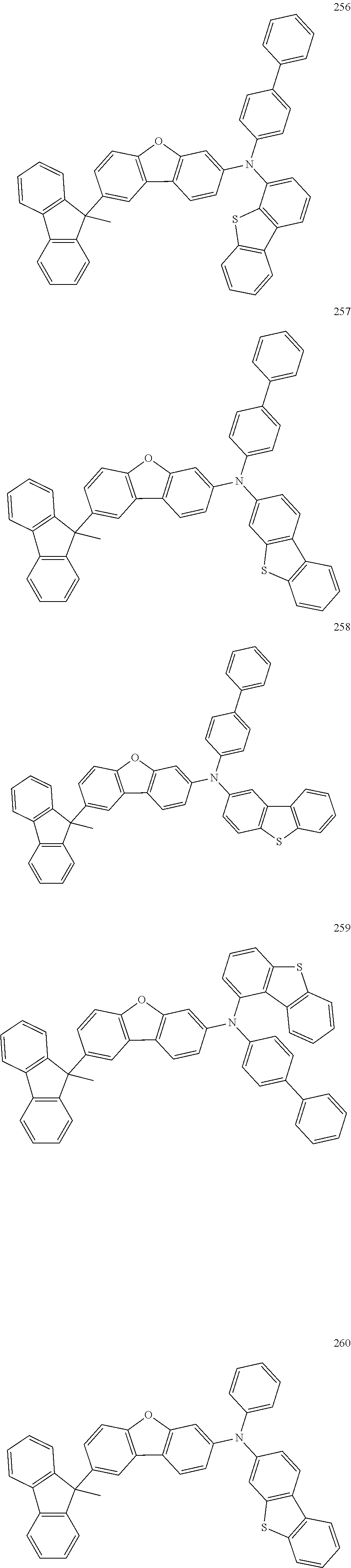 [SMILES: CC1(c2ccc3oc4cc(N(c5ccc(-c6ccccc6)cc5)c5cccc6c5sc5ccccc56)ccc4c3c2)c2ccccc2-c2ccccc21 ; CC1(c2ccc3oc4cc(N(c5ccc(-c6ccccc6)cc5)c5ccc6c(c5)sc5ccccc56)ccc4c3c2)c2ccccc2-c2ccccc21 ; CC1(c2ccc3oc4cc(N(c5ccc(-c6ccccc6)cc5)c5ccc6sc7ccccc7c6c5)ccc4c3c2)c2ccccc2-c2ccccc21 ; CC1(c2ccc3oc4cc(N(c5ccc(-c6ccccc6)cc5)c5cccc6sc7ccccc7c56)ccc4c3c2)c2ccccc2-c2ccccc21 ; CC1(c2ccc3oc4cc(N(c5ccccc5)c5ccc6c(c5)sc5ccccc56)ccc4c3c2)c2ccccc2-c2ccccc21]

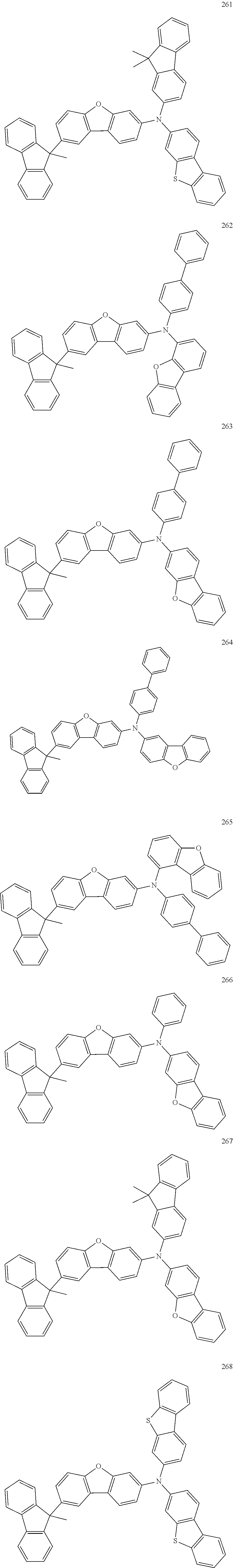

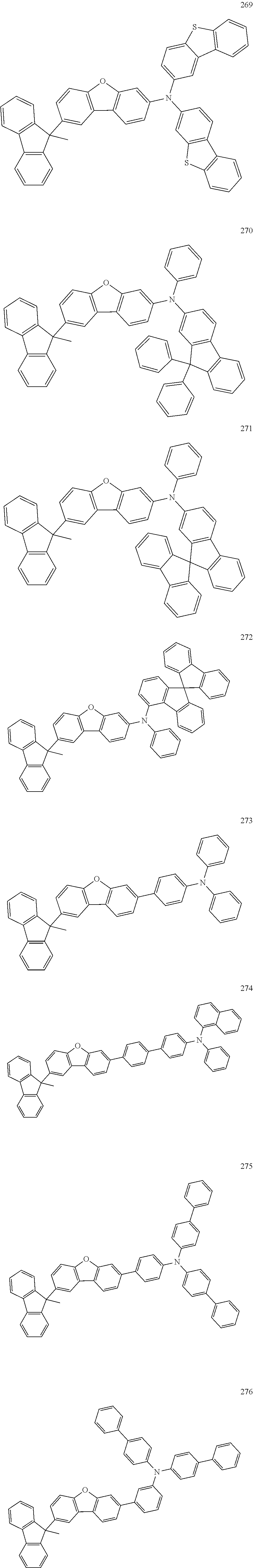

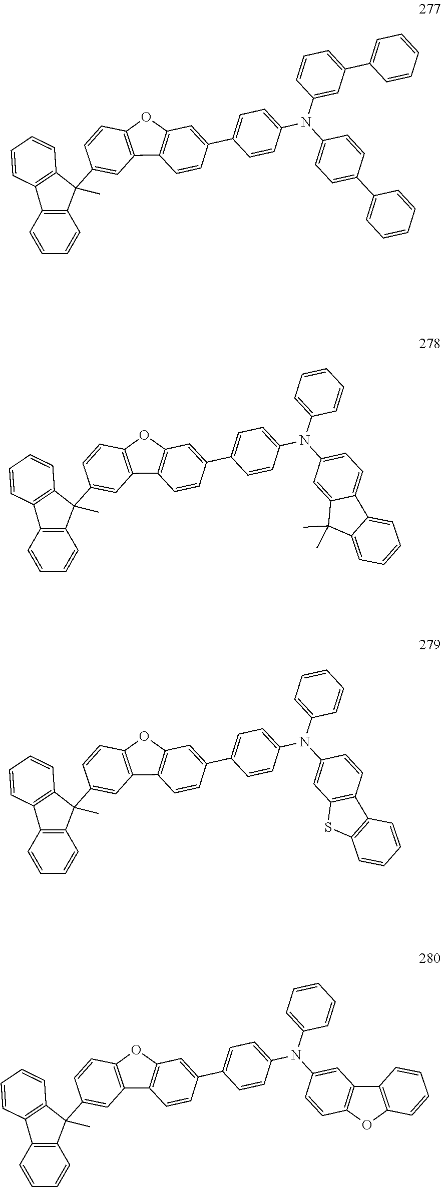 [SMILES: CC1(c2ccc3oc4cc(-c5ccc(N(c6ccc(-c7ccccc7)cc6)c6cccc(-c7ccccc7)c6)cc5)ccc4c3c2)c2ccccc2-c2ccccc21 ; CC1(C)c2ccccc2-c2ccc(N(c3ccccc3)c3ccc(-c4ccc5c(c4)oc4ccc(C6(C)c7ccccc7-c7ccccc76)cc45)cc3)cc21 ; CC1(c2ccc3oc4cc(-c5ccc(N(c6ccccc6)c6ccc7c(c6)sc6ccccc67)cc5)ccc4c3c2)c2ccccc2-c2ccccc21 ; CC1(c2ccc3oc4cc(-c5ccc(N(c6ccccc6)c6ccc7oc8ccccc8c7c6)cc5)ccc4c3c2)c2ccccc2-c2ccccc21]

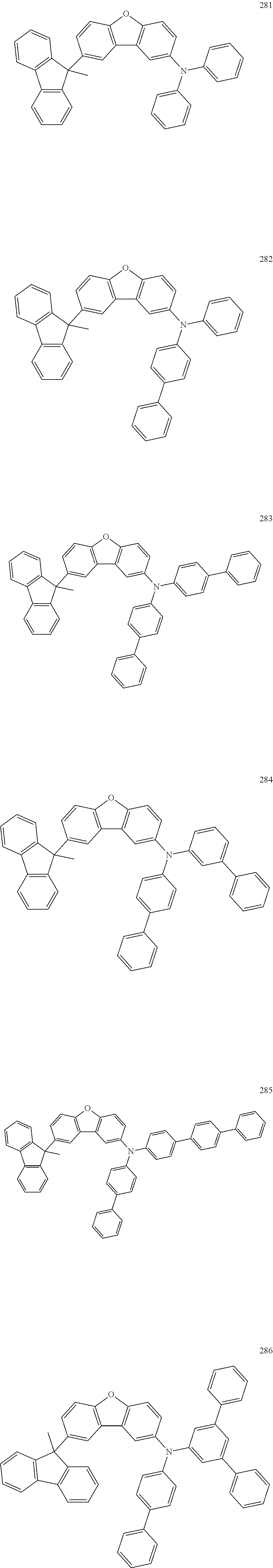

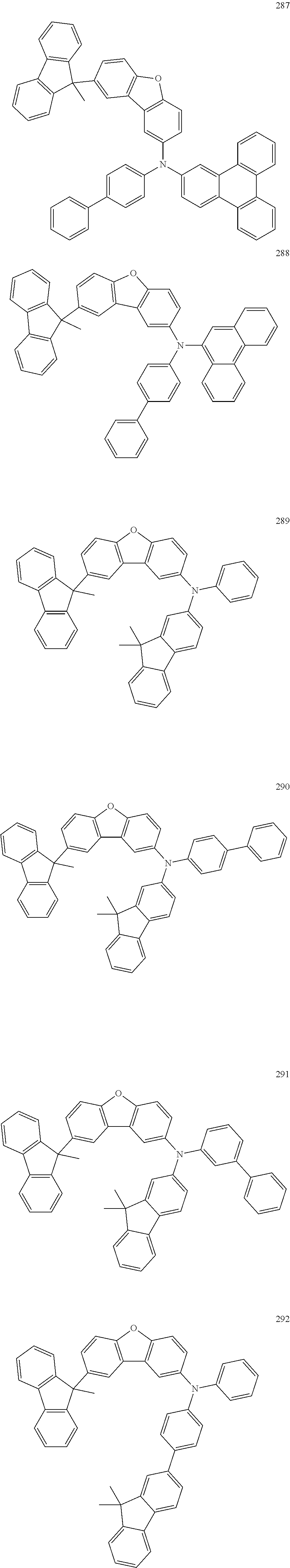

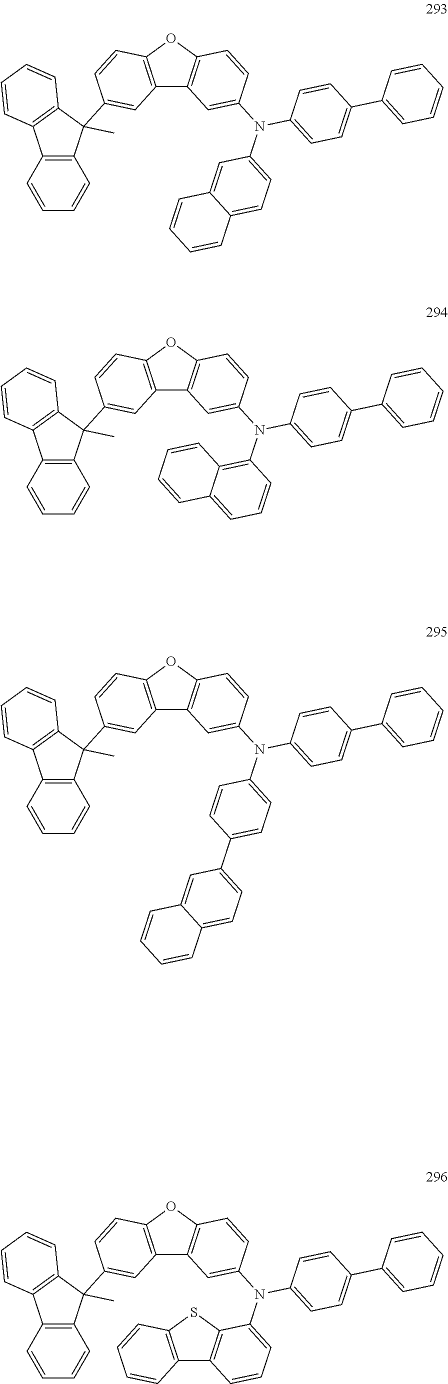 [SMILES: CC1(c2ccc3oc4ccc(N(c5ccc(-c6ccccc6)cc5)c5ccc6ccccc6c5)cc4c3c2)c2ccccc2-c2ccccc21 ; CC1(c2ccc3oc4ccc(N(c5ccc(-c6ccccc6)cc5)c5cccc6ccccc56)cc4c3c2)c2ccccc2-c2ccccc21 ; CC1(c2ccc3oc4ccc(N(c5ccc(-c6ccccc6)cc5)c5ccc(-c6ccc7ccccc7c6)cc5)cc4c3c2)c2ccccc2-c2ccccc21 ; CC1(c2ccc3oc4ccc(N(c5ccc(-c6ccccc6)cc5)c5cccc6c5sc5ccccc56)cc4c3c2)c2ccccc2-c2ccccc21]

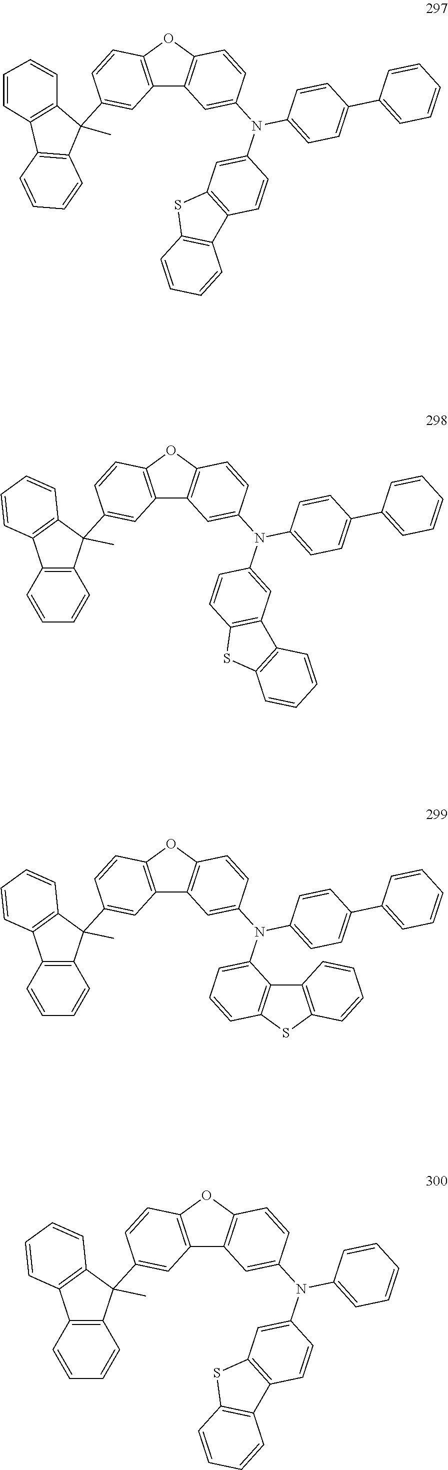 [SMILES: CC1(c2ccc3oc4ccc(N(c5ccc(-c6ccccc6)cc5)c5ccc6c(c5)sc5ccccc56)cc4c3c2)c2ccccc2-c2ccccc21 ; CC1(c2ccc3oc4ccc(N(c5ccc(-c6ccccc6)cc5)c5ccc6sc7ccccc7c6c5)cc4c3c2)c2ccccc2-c2ccccc21 ; CC1(c2ccc3oc4ccc(N(c5ccc(-c6ccccc6)cc5)c5cccc6sc7ccccc7c56)cc4c3c2)c2ccccc2-c2ccccc21 ; CC1(c2ccc3oc4ccc(N(c5ccccc5)c5ccc6c(c5)sc5ccccc56)cc4c3c2)c2ccccc2-c2ccccc21]

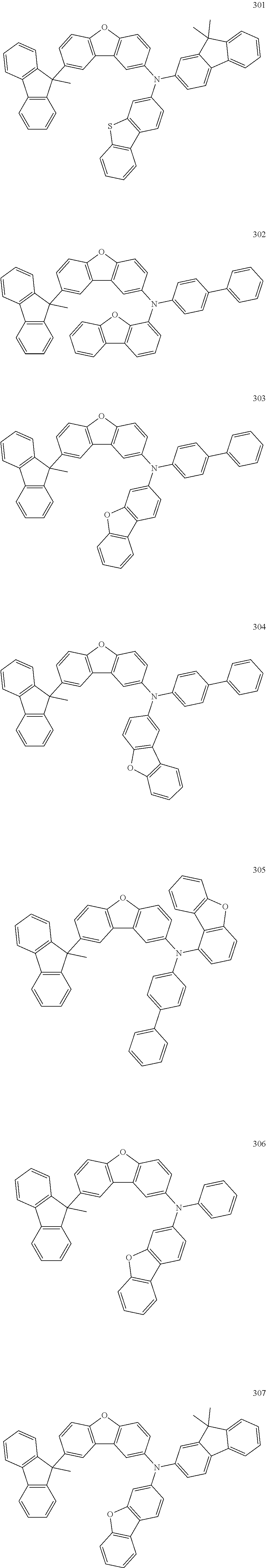

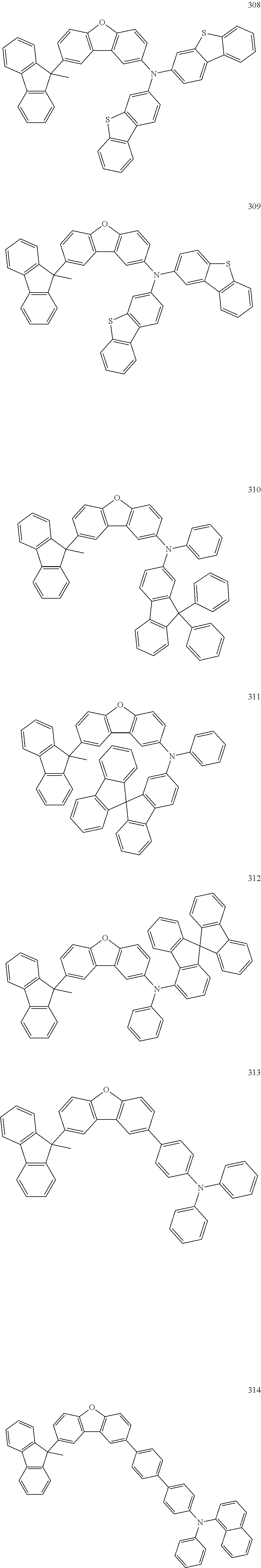

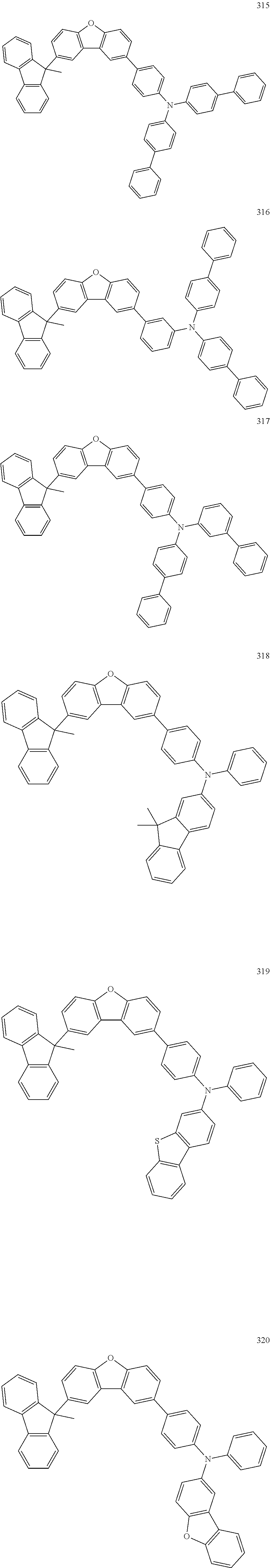

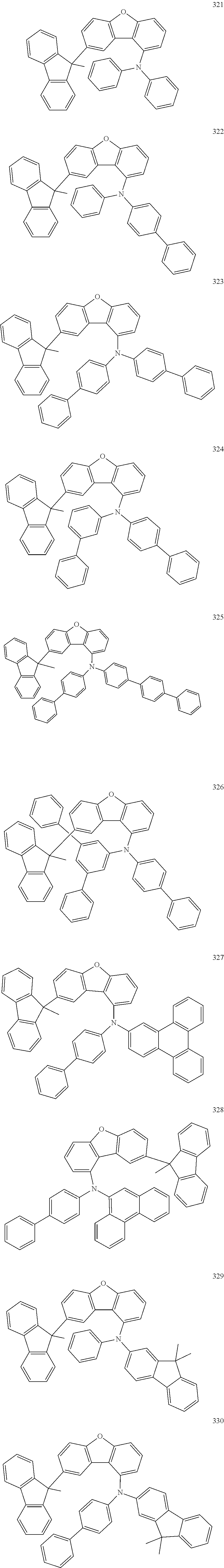

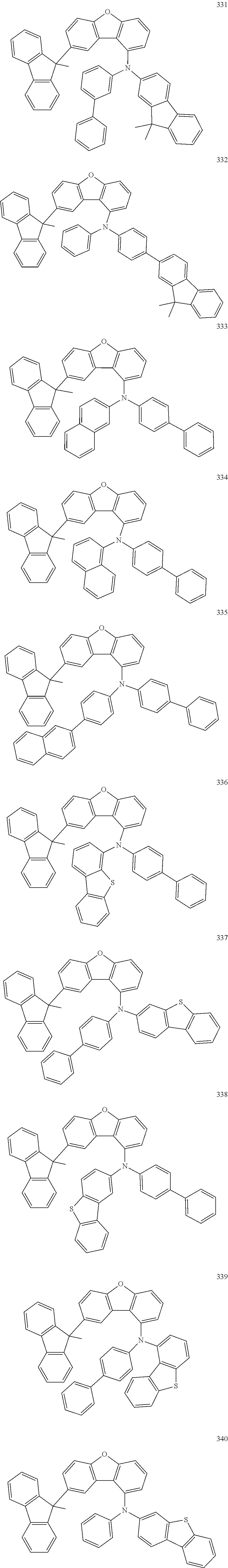

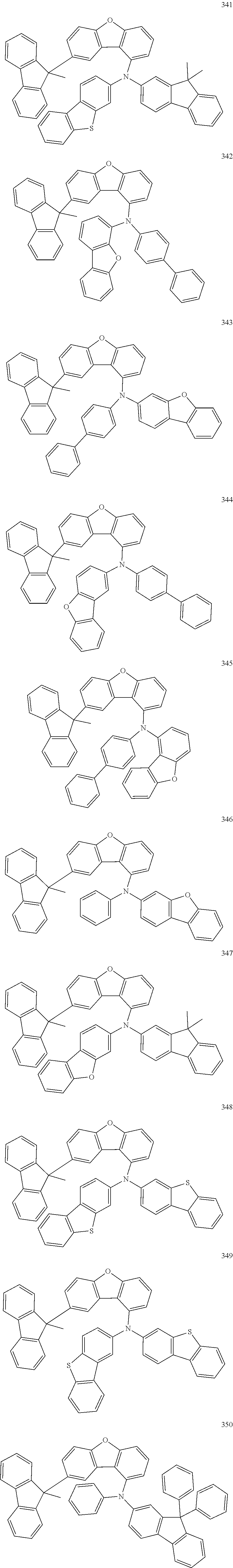

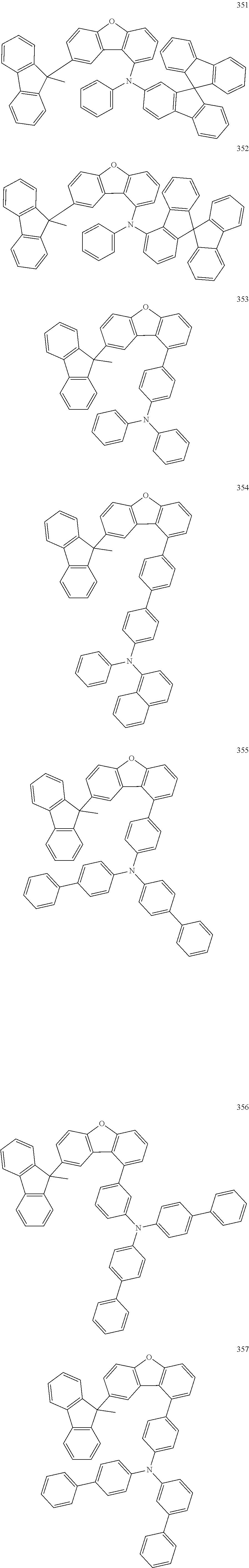

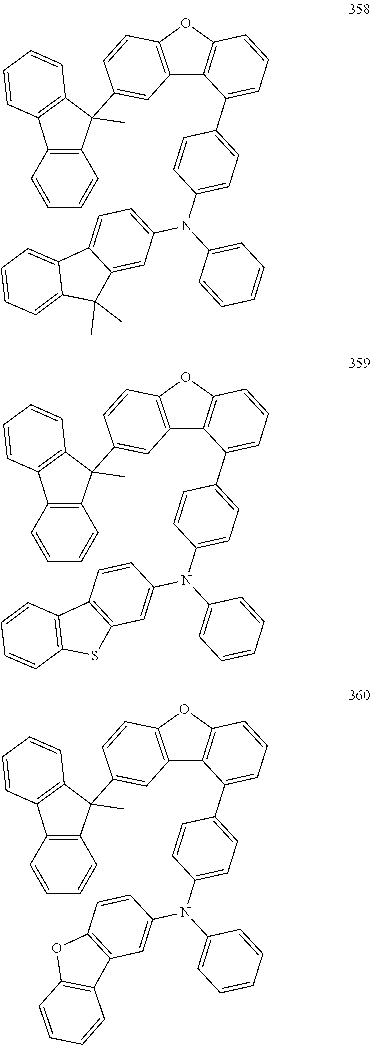 [SMILES: CC1(C)c2ccccc2-c2ccc(N(c3ccccc3)c3ccc(-c4cccc5oc6ccc(C7(C)c8ccccc8-c8ccccc87)cc6c45)cc3)cc21 ; CC1(c2ccc3oc4cccc(-c5ccc(N(c6ccccc6)c6ccc7c(c6)sc6ccccc67)cc5)c4c3c2)c2ccccc2-c2ccccc21 ; CC1(c2ccc3oc4cccc(-c5ccc(N(c6ccccc6)c6ccc7oc8ccccc8c7c6)cc5)c4c3c2)c2ccccc2-c2ccccc21]

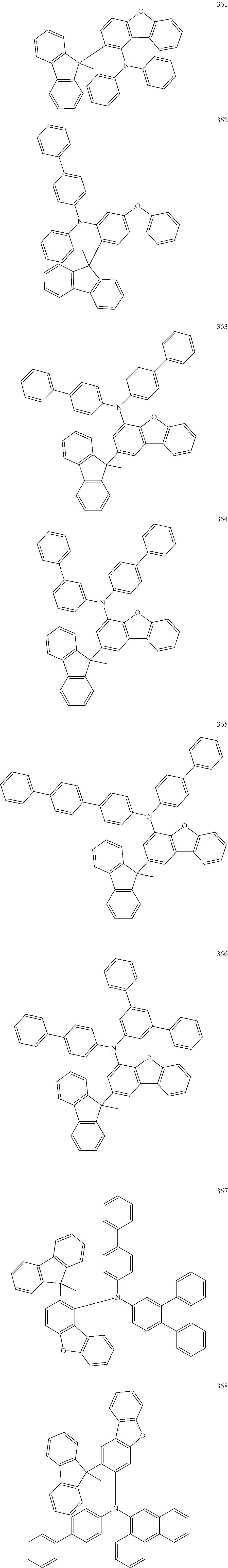

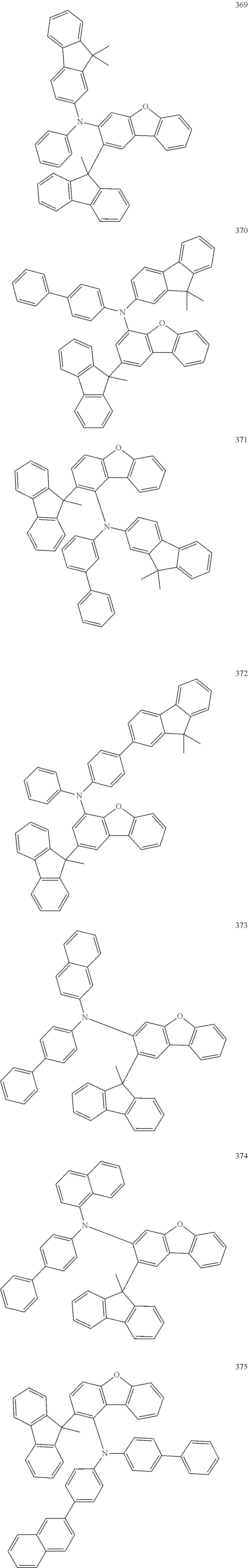

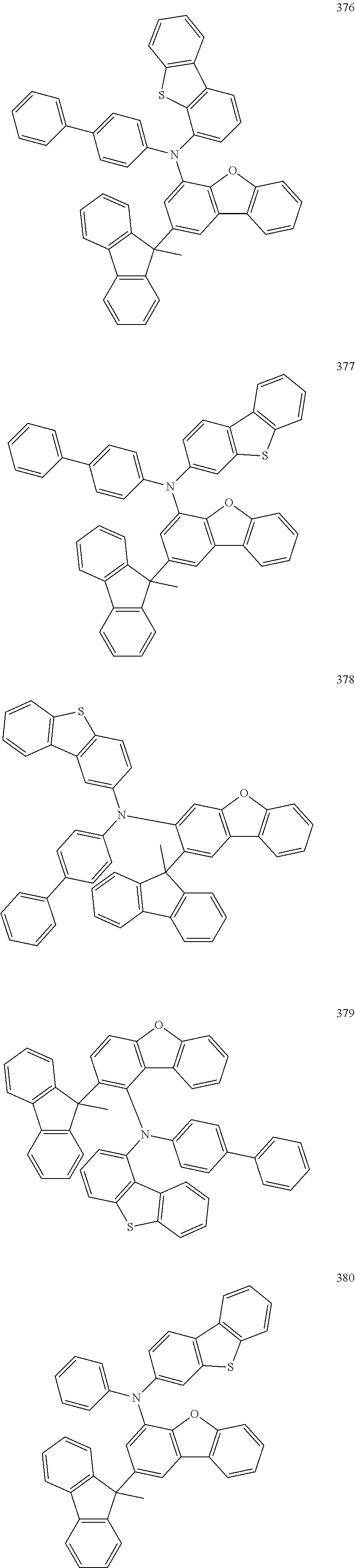 [SMILES: CC1(c2cc(N(c3ccc(-c4ccccc4)cc3)c3cccc4c3sc3ccccc34)c3oc4ccccc4c3c2)c2ccccc2-c2ccccc21 ; CC1(c2cc(N(c3ccc(-c4ccccc4)cc3)c3ccc4c(c3)sc3ccccc34)c3oc4ccccc4c3c2)c2ccccc2-c2ccccc21 ; CC1(c2cc3c(cc2N(c2ccc(-c4ccccc4)cc2)c2ccc4sc5ccccc5c4c2)oc2ccccc23)c2ccccc2-c2ccccc21 ; CC1(c2ccc3oc4ccccc4c3c2N(c2ccc(-c3ccccc3)cc2)c2cccc3sc4ccccc4c23)c2ccccc2-c2ccccc21 ; CC1(c2cc(N(c3ccccc3)c3ccc4c(c3)sc3ccccc34)c3oc4ccccc4c3c2)c2ccccc2-c2ccccc21]

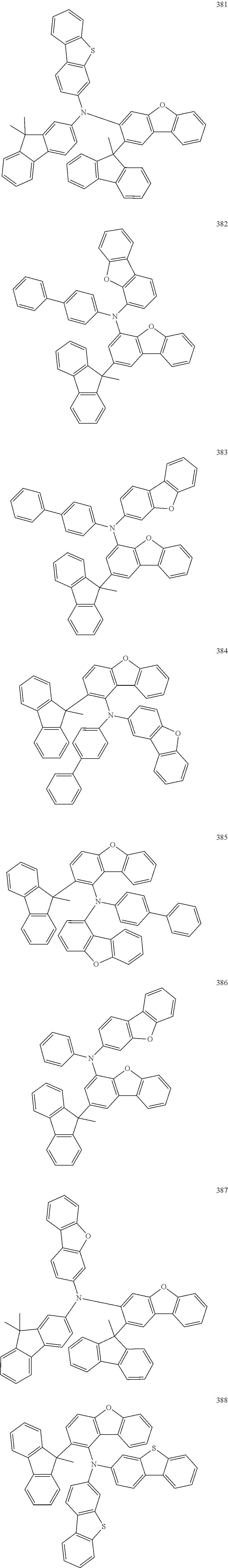

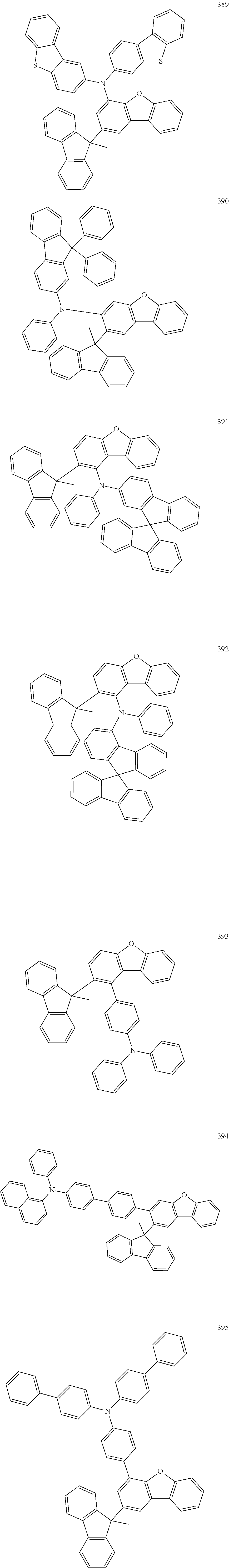

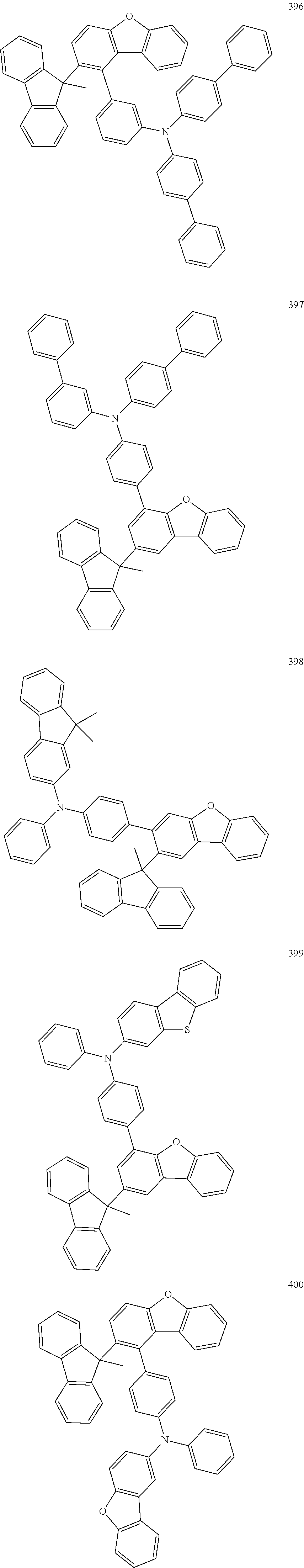 [SMILES: CC1(c2ccc3oc4ccccc4c3c2-c2cccc(N(c3ccc(-c4ccccc4)cc3)c3ccc(-c4ccccc4)cc3)c2)c2ccccc2-c2ccccc21 ; CC1(c2cc(-c3ccc(N(c4ccc(-c5ccccc5)cc4)c4cccc(-c5ccccc5)c4)cc3)c3oc4ccccc4c3c2)c2ccccc2-c2ccccc21 ; CC1(C)c2ccccc2-c2ccc(N(c3ccccc3)c3ccc(-c4cc5oc6ccccc6c5cc4C4(C)c5ccccc5-c5ccccc54)cc3)cc21 ; CC1(c2cc(-c3ccc(N(c4ccccc4)c4ccc5c(c4)sc4ccccc45)cc3)c3oc4ccccc4c3c2)c2ccccc2-c2ccccc21 ; CC1(c2ccc3oc4ccccc4c3c2-c2ccc(N(c3ccccc3)c3ccc4oc5ccccc5c4c3)cc2)c2ccccc2-c2ccccc21]

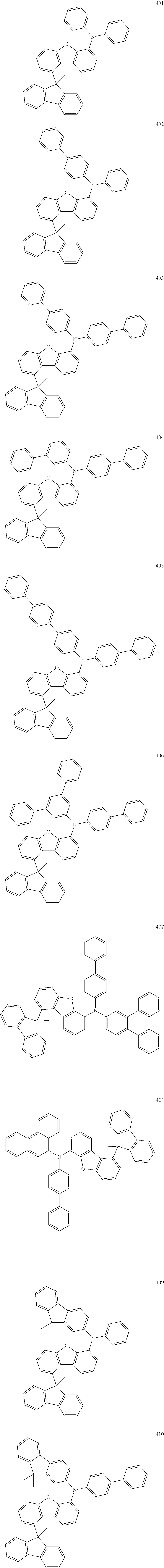

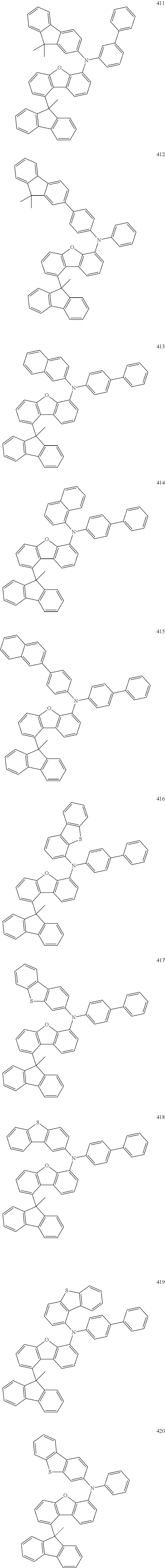

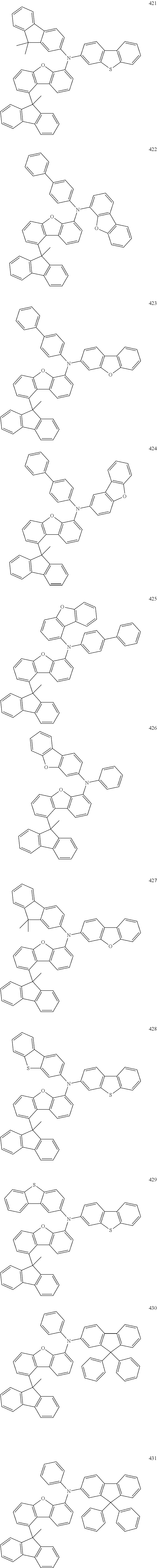

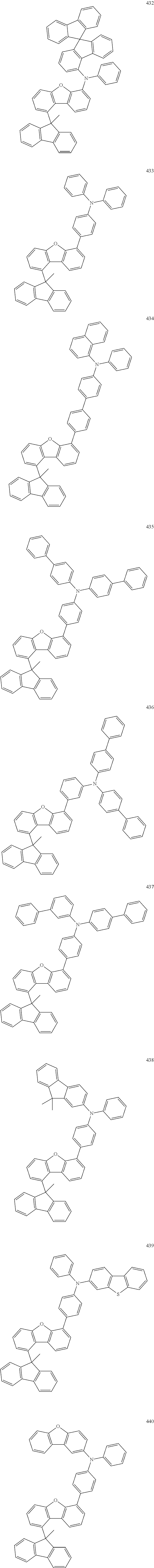

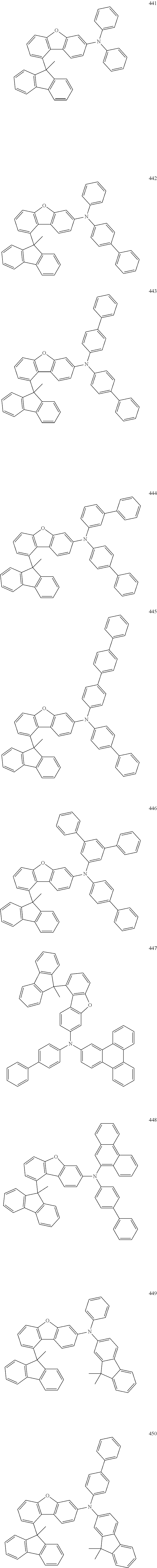

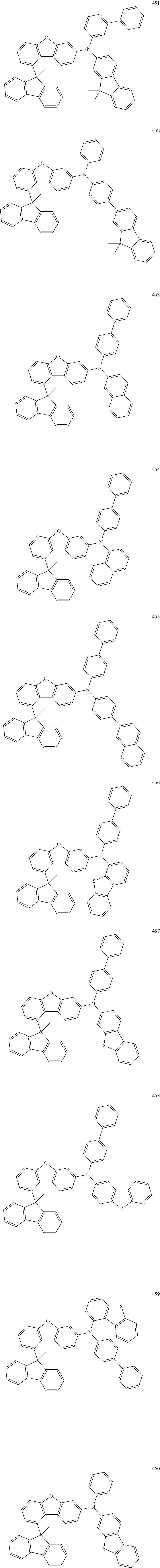

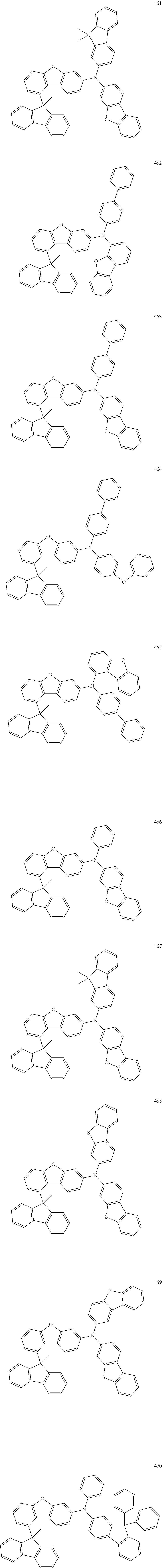

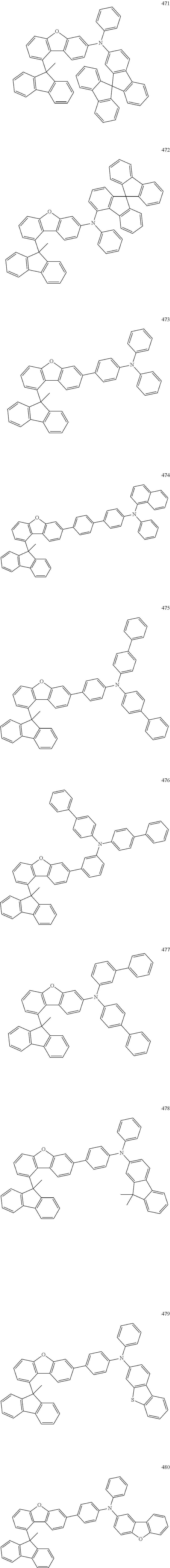

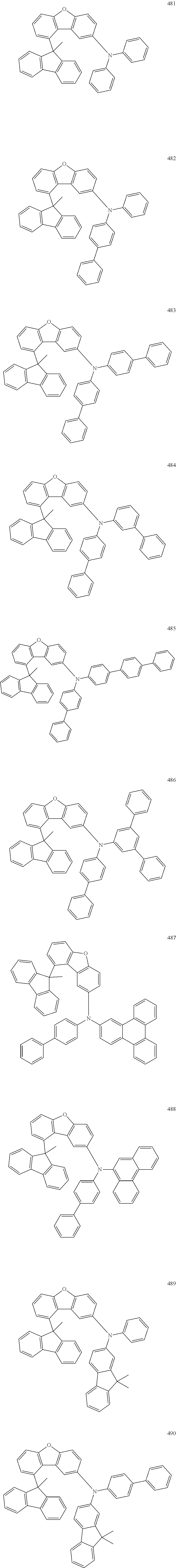

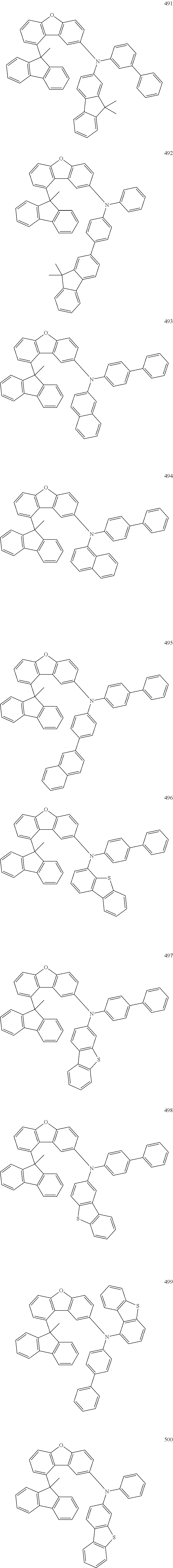

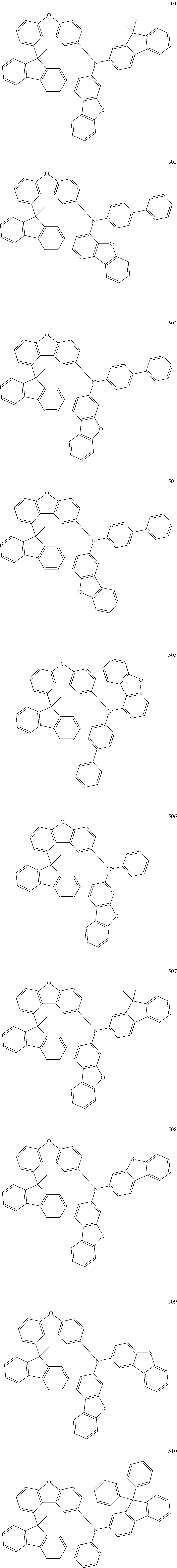

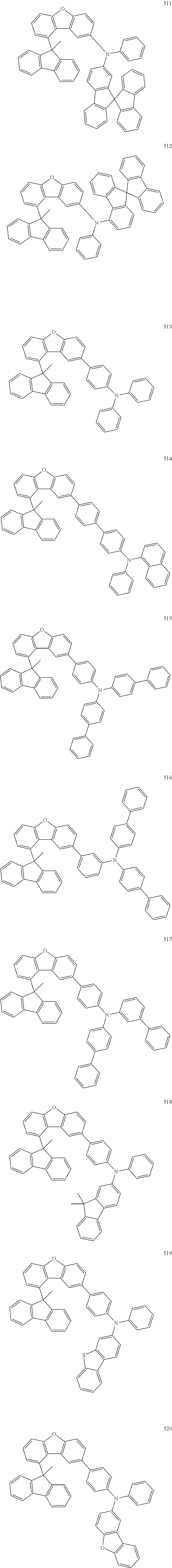

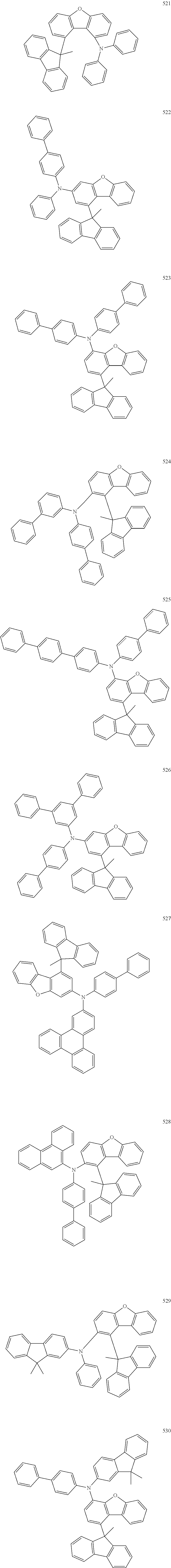

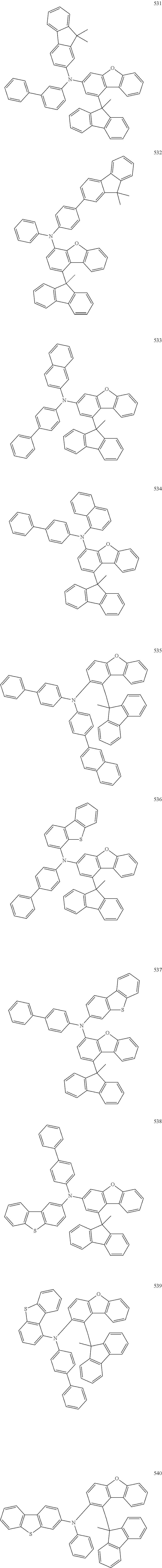

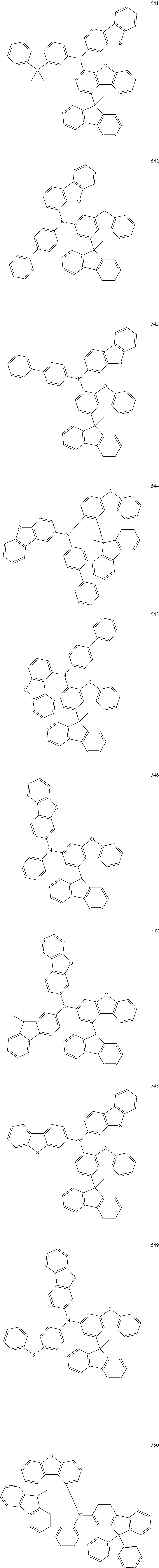

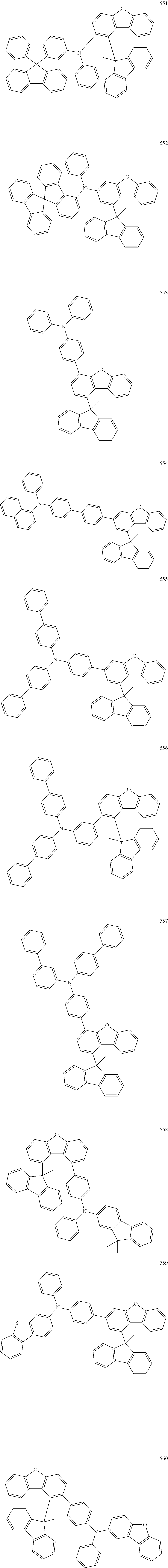

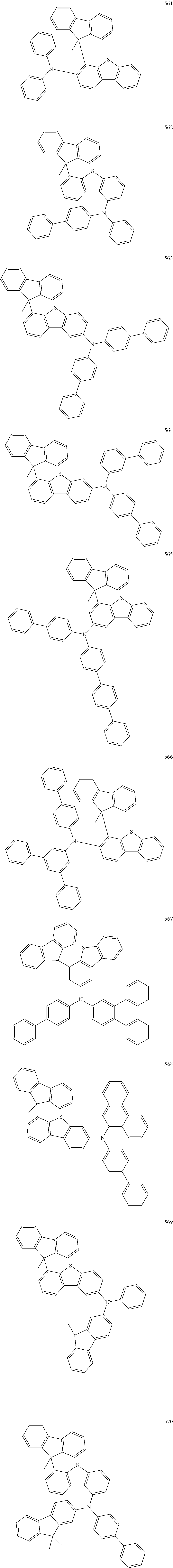

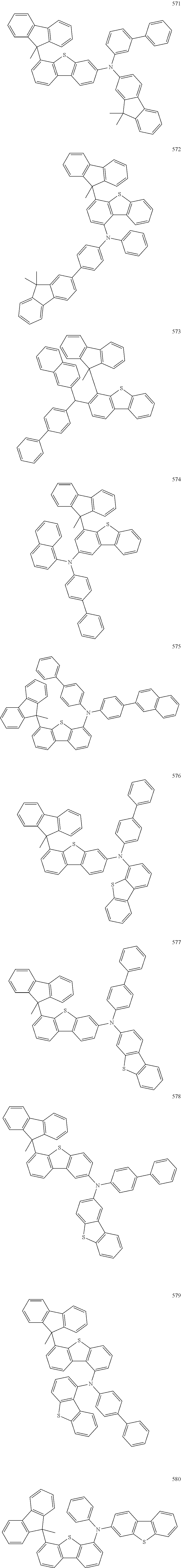

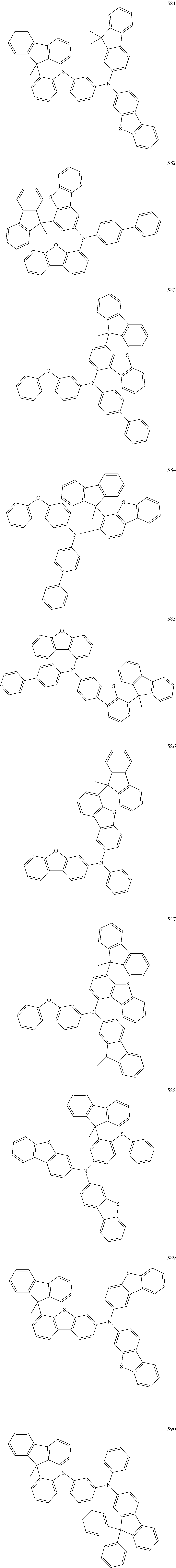

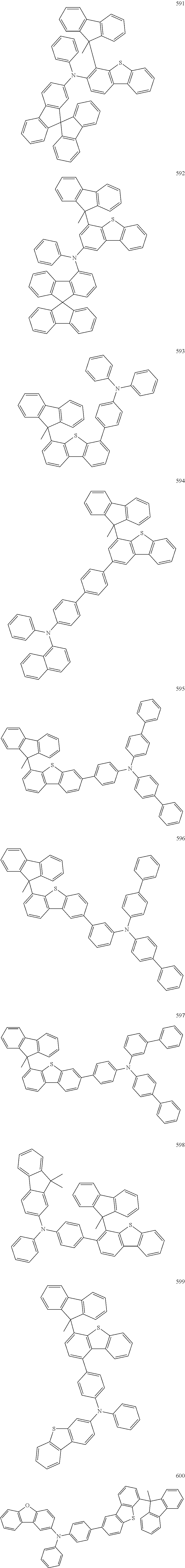

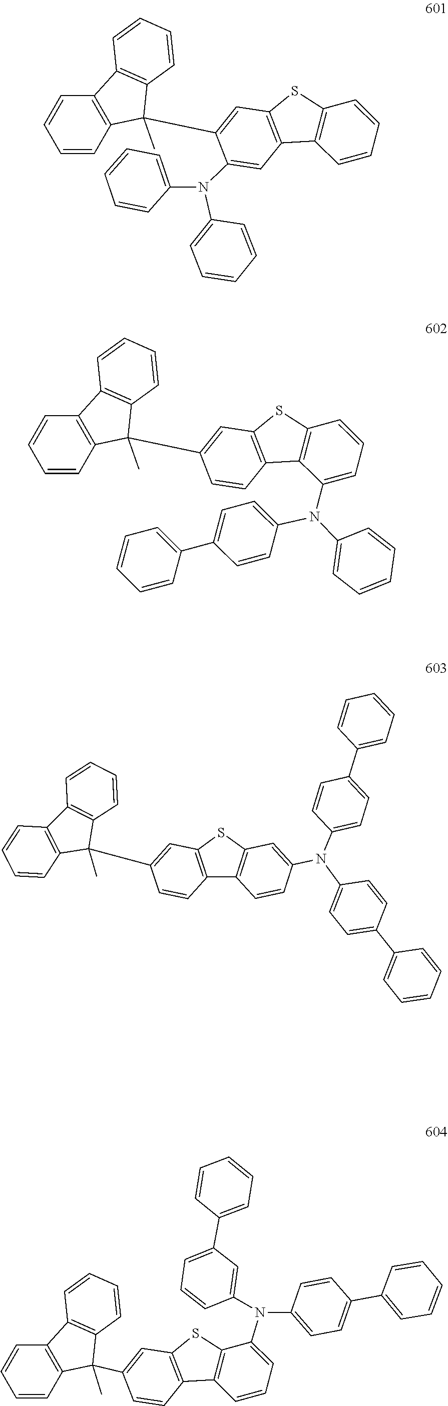 [SMILES: CC1(c2cc3sc4ccccc4c3cc2N(c2ccccc2)c2ccccc2)c2ccccc2-c2ccccc21 ; CC1(c2ccc3c(c2)sc2cccc(N(c4ccccc4)c4ccc(-c5ccccc5)cc4)c23)c2ccccc2-c2ccccc21 ; CC1(c2ccc3c(c2)sc2cc(N(c4ccc(-c5ccccc5)cc4)c4ccc(-c5ccccc5)cc4)ccc23)c2ccccc2-c2ccccc21 ; CC1(c2ccc3c(c2)sc2c(N(c4ccc(-c5ccccc5)cc4)c4cccc(-c5ccccc5)c4)cccc23)c2ccccc2-c2ccccc21]

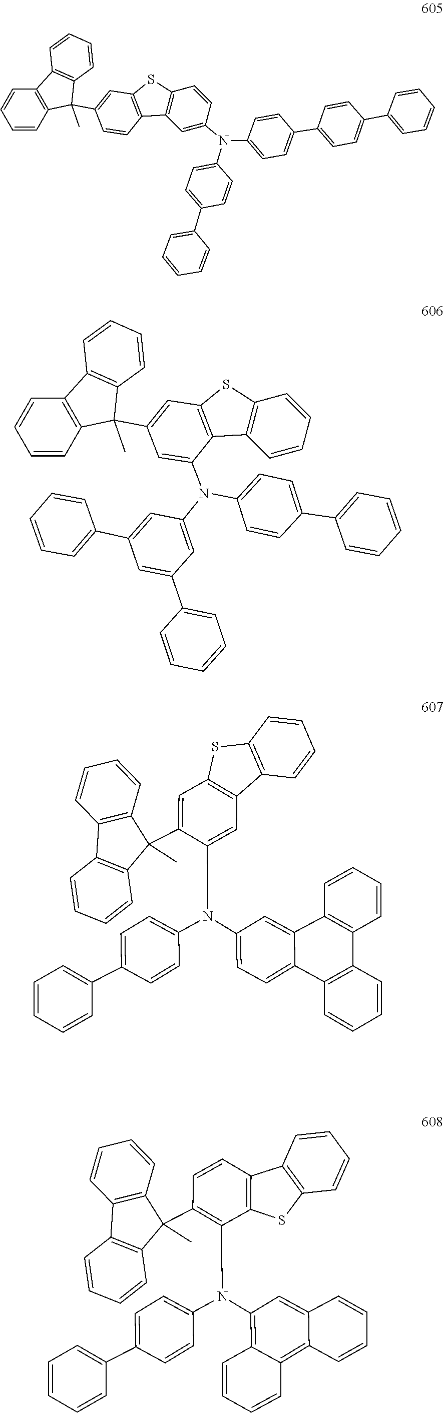 [SMILES: CC1(c2ccc3c(c2)sc2ccc(N(c4ccc(-c5ccccc5)cc4)c4ccc(-c5ccc(-c6ccccc6)cc5)cc4)cc23)c2ccccc2-c2ccccc21 ; CC1(c2cc(N(c3ccc(-c4ccccc4)cc3)c3cc(-c4ccccc4)cc(-c4ccccc4)c3)c3c(c2)sc2ccccc23)c2ccccc2-c2ccccc21 ; CC1(c2cc3sc4ccccc4c3cc2N(c2ccc(-c3ccccc3)cc2)c2ccc3c4ccccc4c4ccccc4c3c2)c2ccccc2-c2ccccc21 ; CC1(c2ccc3c(sc4ccccc43)c2N(c2ccc(-c3ccccc3)cc2)c2cc3ccccc3c3ccccc23)c2ccccc2-c2ccccc21]

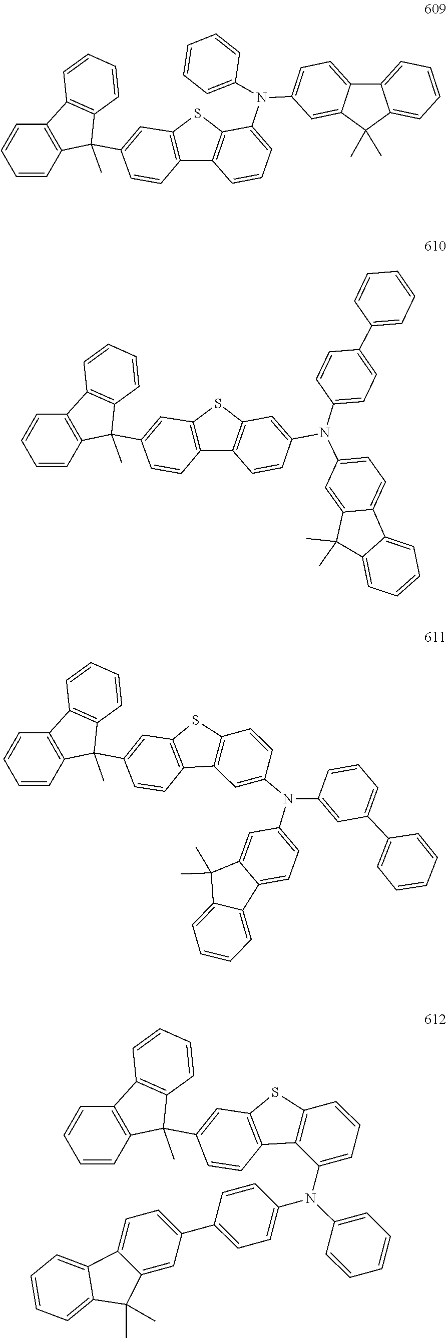 [SMILES: CC1(C)c2ccccc2-c2ccc(N(c3ccccc3)c3cccc4c3sc3cc(C5(C)c6ccccc6-c6ccccc65)ccc34)cc21 ; CC1(C)c2ccccc2-c2ccc(N(c3ccc(-c4ccccc4)cc3)c3ccc4c(c3)sc3cc(C5(C)c6ccccc6-c6ccccc65)ccc34)cc21 ; CC1(C)c2ccccc2-c2ccc(N(c3cccc(-c4ccccc4)c3)c3ccc4sc5cc(C6(C)c7ccccc7-c7ccccc76)ccc5c4c3)cc21 ; CC1(C)c2ccccc2-c2ccc(-c3ccc(N(c4ccccc4)c4cccc5sc6cc(C7(C)c8ccccc8-c8ccccc87)ccc6c45)cc3)cc21]

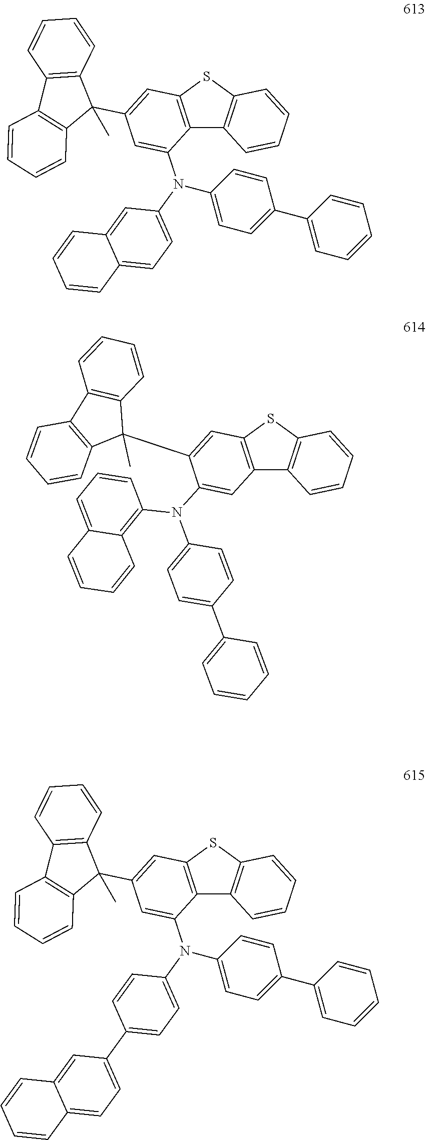

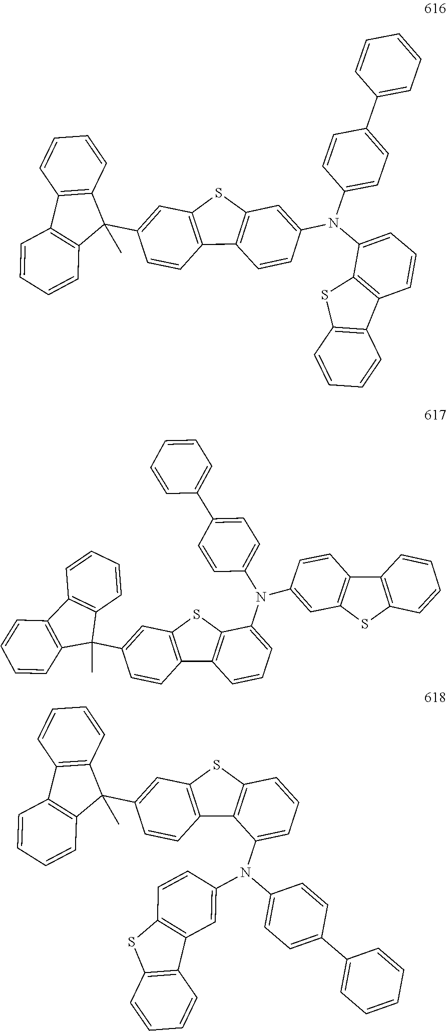

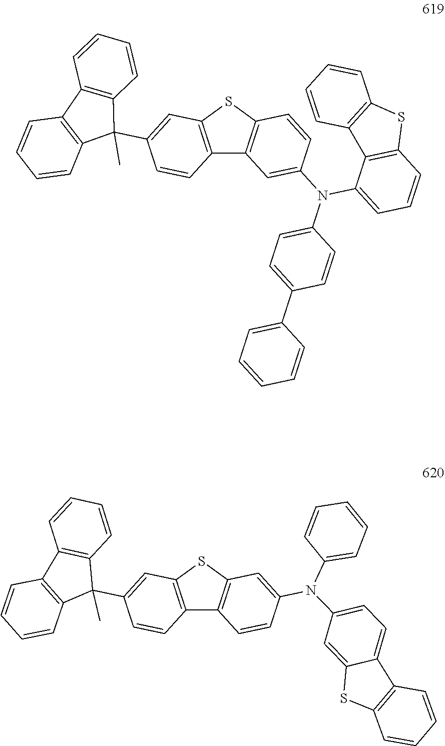 [SMILES: CC1(c2ccc3c(c2)sc2ccc(N(c4ccc(-c5ccccc5)cc4)c4cccc5sc6ccccc6c45)cc23)c2ccccc2-c2ccccc21 ; CC1(c2ccc3c(c2)sc2cc(N(c4ccccc4)c4ccc5c(c4)sc4ccccc45)ccc23)c2ccccc2-c2ccccc21]

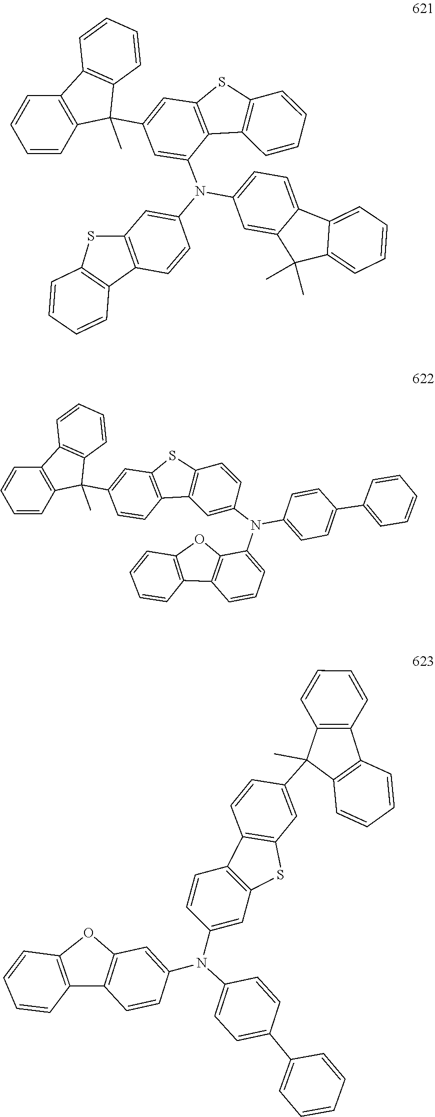 [SMILES: CC1(C)c2ccccc2-c2ccc(N(c3ccc4c(c3)sc3ccccc34)c3cc(C4(C)c5ccccc5-c5ccccc54)cc4sc5ccccc5c34)cc21 ; CC1(c2ccc3c(c2)sc2ccc(N(c4ccc(-c5ccccc5)cc4)c4cccc5c4oc4ccccc45)cc23)c2ccccc2-c2ccccc21 ; CC1(c2ccc3c(c2)sc2cc(N(c4ccc(-c5ccccc5)cc4)c4ccc5c(c4)oc4ccccc45)ccc23)c2ccccc2-c2ccccc21]

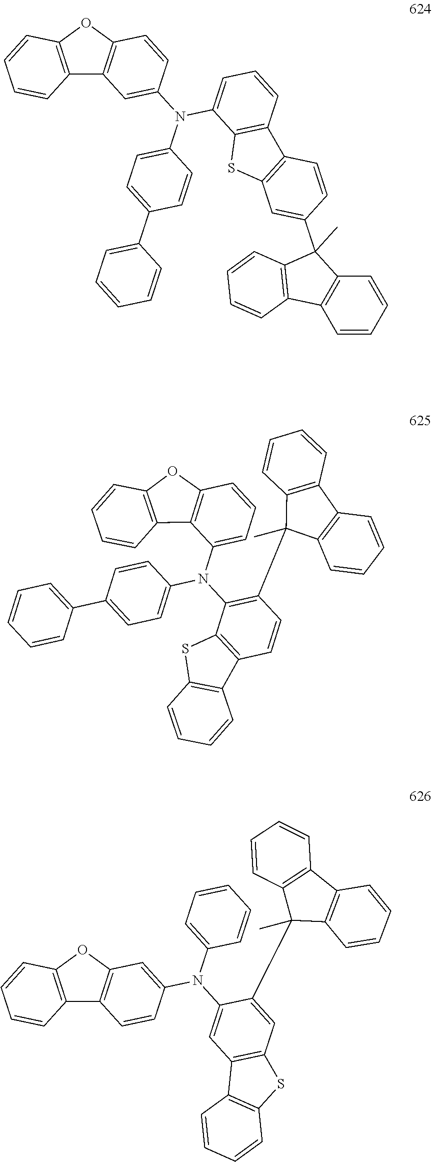 [SMILES: CC1(c2ccc3c(c2)sc2c(N(c4ccc(-c5ccccc5)cc4)c4ccc5oc6ccccc6c5c4)cccc23)c2ccccc2-c2ccccc21 ; CC1(c2ccc3c(sc4ccccc43)c2N(c2ccc(-c3ccccc3)cc2)c2cccc3oc4ccccc4c23)c2ccccc2-c2ccccc21 ; CC1(c2cc3sc4ccccc4c3cc2N(c2ccccc2)c2ccc3c(c2)oc2ccccc23)c2ccccc2-c2ccccc21]

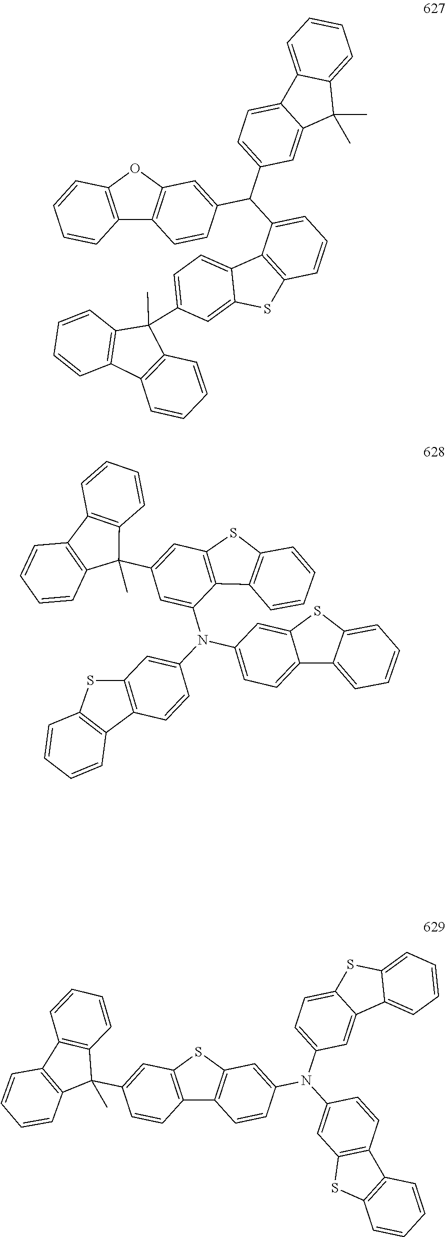 [SMILES: CC1(C)c2ccccc2-c2ccc(C(c3ccc4c(c3)oc3ccccc34)c3cccc4sc5cc(C6(C)c7ccccc7-c7ccccc76)ccc5c34)cc21 ; CC1(c2cc(N(c3ccc4c(c3)sc3ccccc34)c3ccc4c(c3)sc3ccccc34)c3c(c2)sc2ccccc23)c2ccccc2-c2ccccc21 ; CC1(c2ccc3c(c2)sc2cc(N(c4ccc5c(c4)sc4ccccc45)c4ccc5sc6ccccc6c5c4)ccc23)c2ccccc2-c2ccccc21]

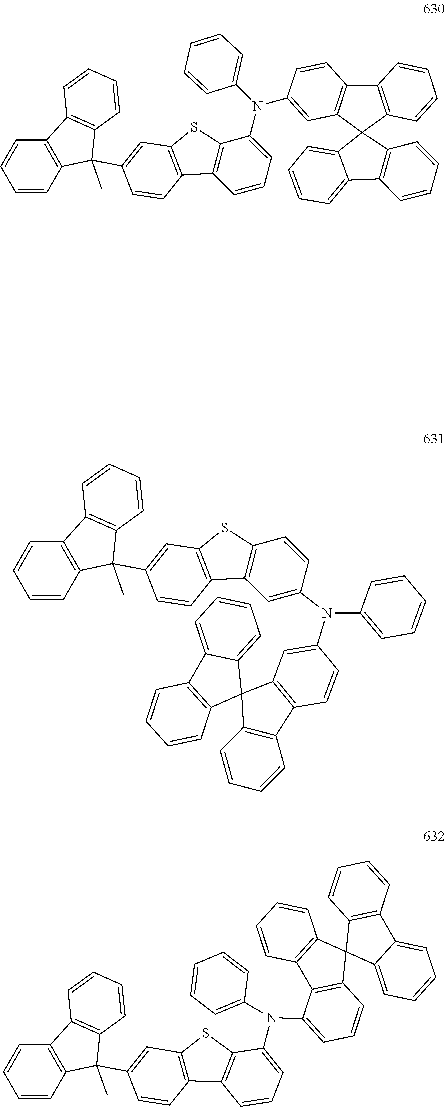 [SMILES: CC1(c2ccc3c(c2)sc2c(N(c4ccccc4)c4ccc5c(c4)C4(c6ccccc6-c6ccccc64)c4ccccc4-5)cccc23)c2ccccc2-c2ccccc21 ; CC1(c2ccc3c(c2)sc2ccc(N(c4ccccc4)c4ccc5c(c4)C4(c6ccccc6-c6ccccc64)c4ccccc4-5)cc23)c2ccccc2-c2ccccc21 ; CC1(c2ccc3c(c2)sc2c(N(c4ccccc4)c4cccc5c4-c4ccccc4C54c5ccccc5-c5ccccc54)cccc23)c2ccccc2-c2ccccc21]

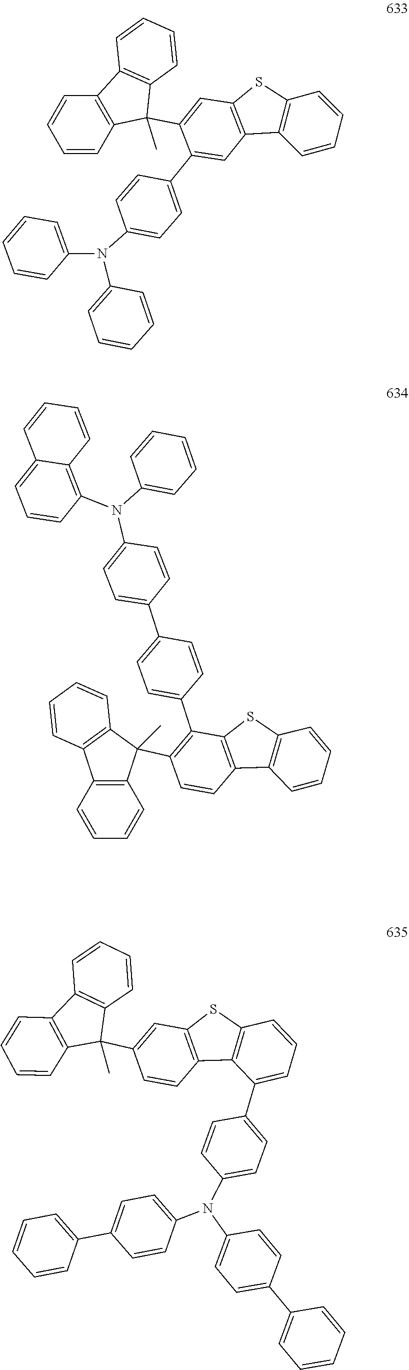 [SMILES: CC1(c2cc3sc4ccccc4c3cc2-c2ccc(N(c3ccccc3)c3ccccc3)cc2)c2ccccc2-c2ccccc21 ; CC1(c2ccc3c(sc4ccccc43)c2-c2ccc(-c3ccc(N(c4ccccc4)c4cccc5ccccc45)cc3)cc2)c2ccccc2-c2ccccc21 ; CC1(c2ccc3c(c2)sc2cccc(-c4ccc(N(c5ccc(-c6ccccc6)cc5)c5ccc(-c6ccccc6)cc5)cc4)c23)c2ccccc2-c2ccccc21]

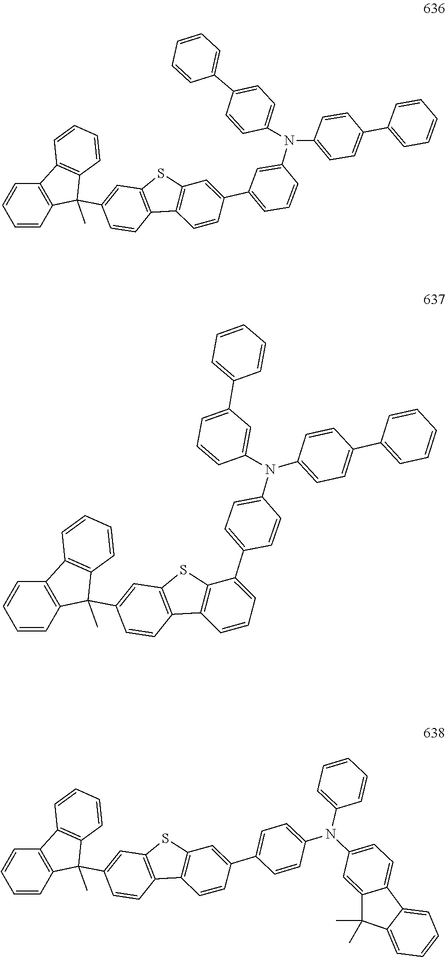 [SMILES: CC1(c2ccc3c(c2)sc2cc(-c4cccc(N(c5ccc(-c6ccccc6)cc5)c5ccc(-c6ccccc6)cc5)c4)ccc23)c2ccccc2-c2ccccc21 ; CC1(c2ccc3c(c2)sc2c(-c4ccc(N(c5ccc(-c6ccccc6)cc5)c5cccc(-c6ccccc6)c5)cc4)cccc23)c2ccccc2-c2ccccc21 ; CC1(C)c2ccccc2-c2ccc(N(c3ccccc3)c3ccc(-c4ccc5c(c4)sc4cc(C6(C)c7ccccc7-c7ccccc76)ccc45)cc3)cc21]

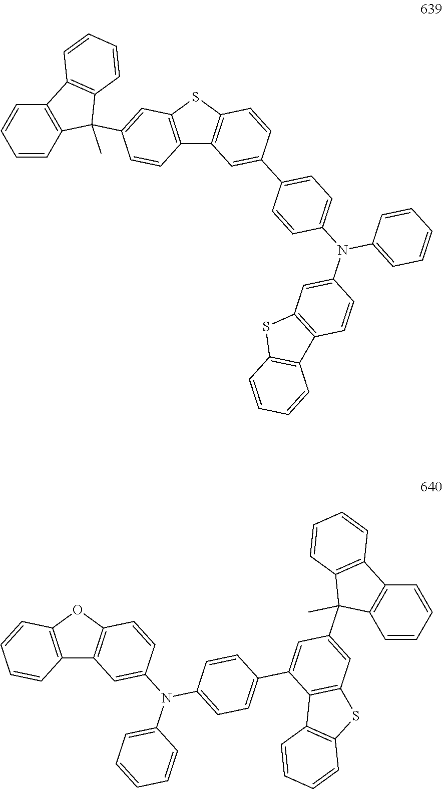 [SMILES: CC1(c2ccc3c(c2)sc2ccc(-c4ccc(N(c5ccccc5)c5ccc6c(c5)sc5ccccc56)cc4)cc23)c2ccccc2-c2ccccc21 ; CC1(c2cc(-c3ccc(N(c4ccccc4)c4ccc5oc6ccccc6c5c4)cc3)c3c(c2)sc2ccccc23)c2ccccc2-c2ccccc21]

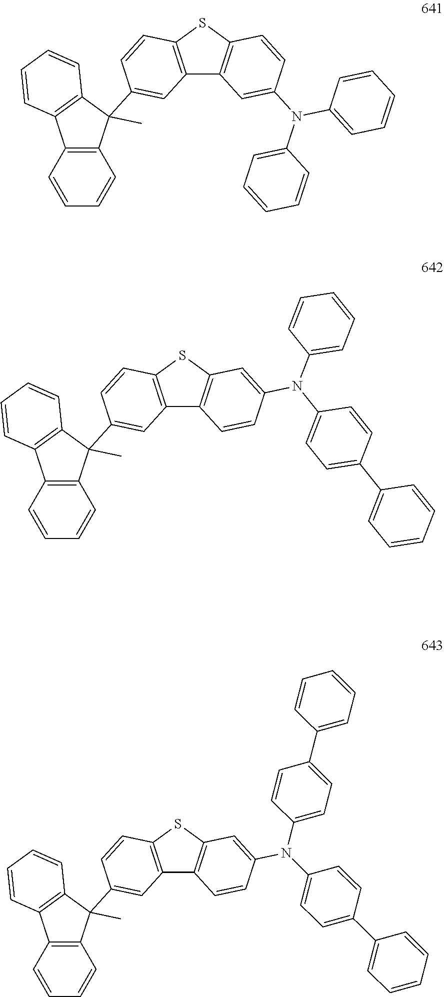 [SMILES: CC1(c2ccc3sc4ccc(N(c5ccccc5)c5ccccc5)cc4c3c2)c2ccccc2-c2ccccc21 ; CC1(c2ccc3sc4cc(N(c5ccccc5)c5ccc(-c6ccccc6)cc5)ccc4c3c2)c2ccccc2-c2ccccc21 ; CC1(c2ccc3sc4cc(N(c5ccc(-c6ccccc6)cc5)c5ccc(-c6ccccc6)cc5)ccc4c3c2)c2ccccc2-c2ccccc21]

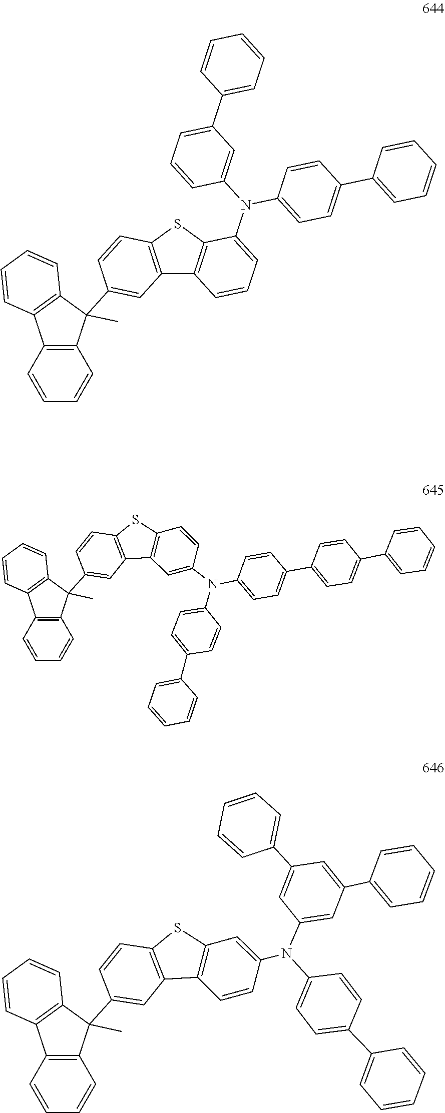 [SMILES: CC1(c2ccc3sc4c(N(c5ccc(-c6ccccc6)cc5)c5cccc(-c6ccccc6)c5)cccc4c3c2)c2ccccc2-c2ccccc21 ; CC1(c2ccc3sc4ccc(N(c5ccc(-c6ccccc6)cc5)c5ccc(-c6ccc(-c7ccccc7)cc6)cc5)cc4c3c2)c2ccccc2-c2ccccc21 ; CC1(c2ccc3sc4cc(N(c5ccc(-c6ccccc6)cc5)c5cc(-c6ccccc6)cc(-c6ccccc6)c5)ccc4c3c2)c2ccccc2-c2ccccc21]

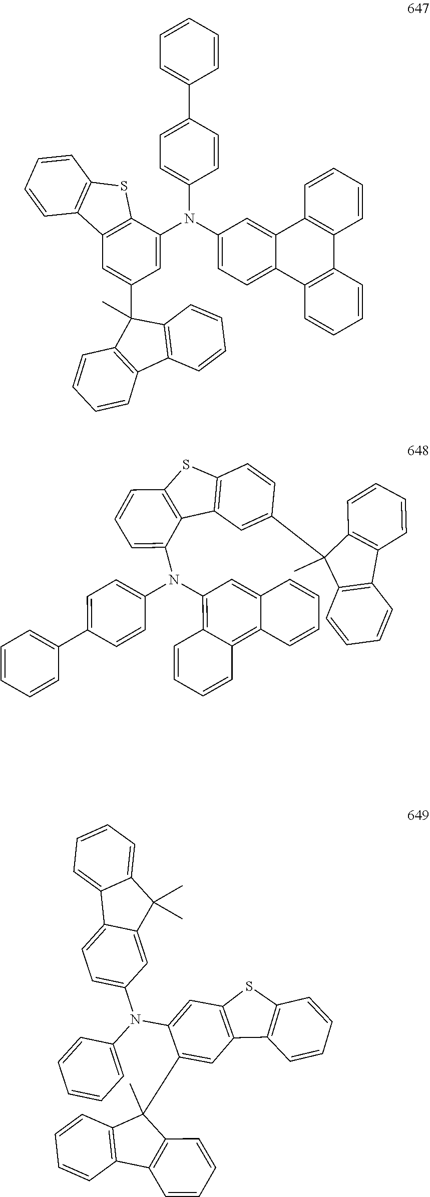 [SMILES: CC1(c2cc(N(c3ccc(-c4ccccc4)cc3)c3ccc4c5ccccc5c5ccccc5c4c3)c3sc4ccccc4c3c2)c2ccccc2-c2ccccc21 ; CC1(c2ccc3sc4cccc(N(c5ccc(-c6ccccc6)cc5)c5cc6ccccc6c6ccccc56)c4c3c2)c2ccccc2-c2ccccc21 ; CC1(C)c2ccccc2-c2ccc(N(c3ccccc3)c3cc4sc5ccccc5c4cc3C3(C)c4ccccc4-c4ccccc43)cc21]

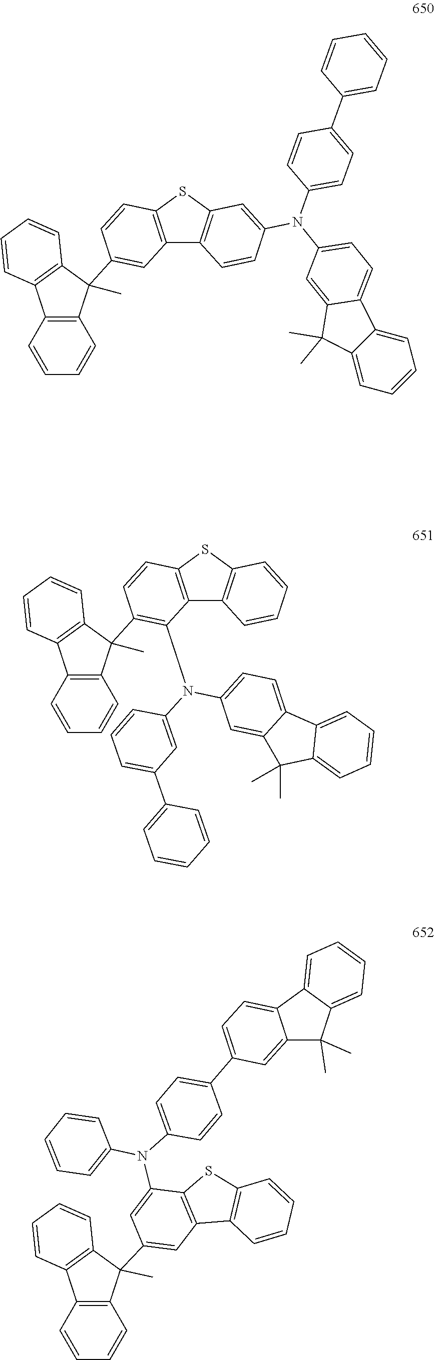 [SMILES: CC1(C)c2ccccc2-c2ccc(N(c3ccc(-c4ccccc4)cc3)c3ccc4c(c3)sc3ccc(C5(C)c6ccccc6-c6ccccc65)cc34)cc21 ; CC1(C)c2ccccc2-c2ccc(N(c3cccc(-c4ccccc4)c3)c3c(C4(C)c5ccccc5-c5ccccc54)ccc4sc5ccccc5c34)cc21 ; CC1(C)c2ccccc2-c2ccc(-c3ccc(N(c4ccccc4)c4cc(C5(C)c6ccccc6-c6ccccc65)cc5c4sc4ccccc45)cc3)cc21]

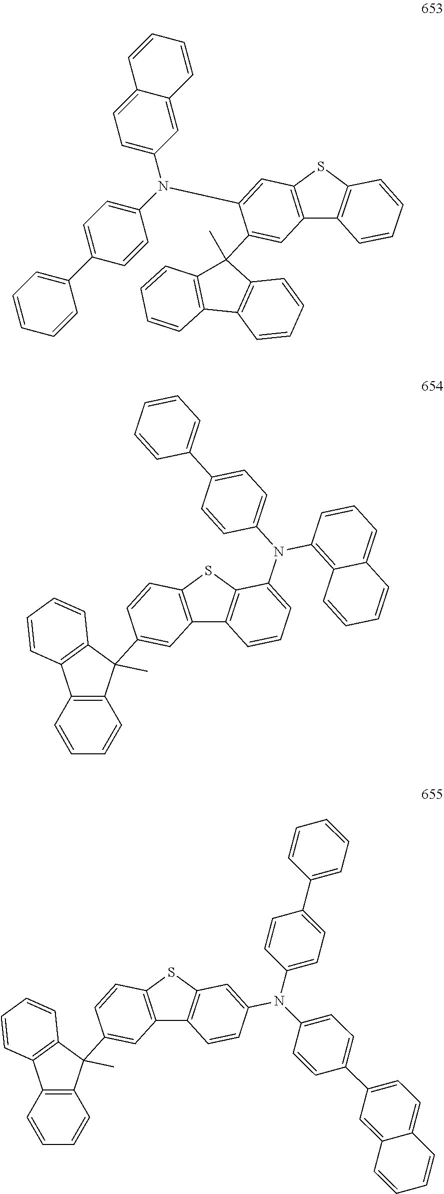 [SMILES: CC1(c2cc3c(cc2N(c2ccc(-c4ccccc4)cc2)c2ccc4ccccc4c2)sc2ccccc23)c2ccccc2-c2ccccc21 ; CC1(c2ccc3sc4c(N(c5ccc(-c6ccccc6)cc5)c5cccc6ccccc56)cccc4c3c2)c2ccccc2-c2ccccc21 ; CC1(c2ccc3sc4cc(N(c5ccc(-c6ccccc6)cc5)c5ccc(-c6ccc7ccccc7c6)cc5)ccc4c3c2)c2ccccc2-c2ccccc21]

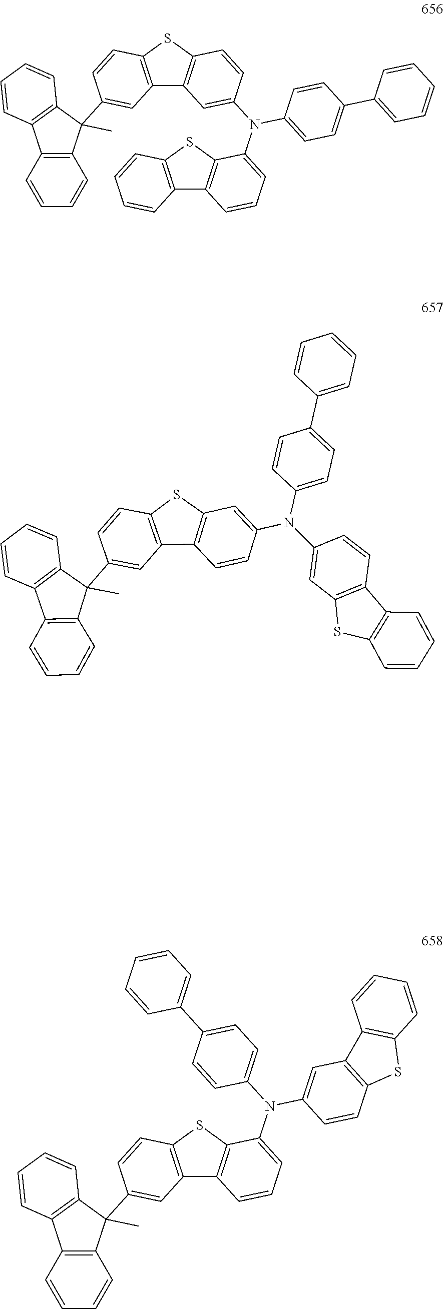 [SMILES: CC1(c2ccc3sc4ccc(N(c5ccc(-c6ccccc6)cc5)c5cccc6c5sc5ccccc56)cc4c3c2)c2ccccc2-c2ccccc21 ; CC1(c2ccc3sc4cc(N(c5ccc(-c6ccccc6)cc5)c5ccc6c(c5)sc5ccccc56)ccc4c3c2)c2ccccc2-c2ccccc21 ; CC1(c2ccc3sc4c(N(c5ccc(-c6ccccc6)cc5)c5ccc6sc7ccccc7c6c5)cccc4c3c2)c2ccccc2-c2ccccc21]

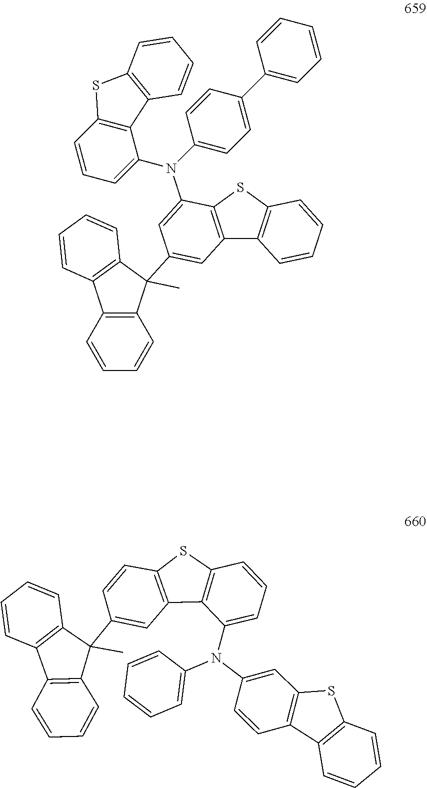 [SMILES: CC1(c2cc(N(c3ccc(-c4ccccc4)cc3)c3cccc4sc5ccccc5c34)c3sc4ccccc4c3c2)c2ccccc2-c2ccccc21 ; CC1(c2ccc3sc4cccc(N(c5ccccc5)c5ccc6c(c5)sc5ccccc56)c4c3c2)c2ccccc2-c2ccccc21]

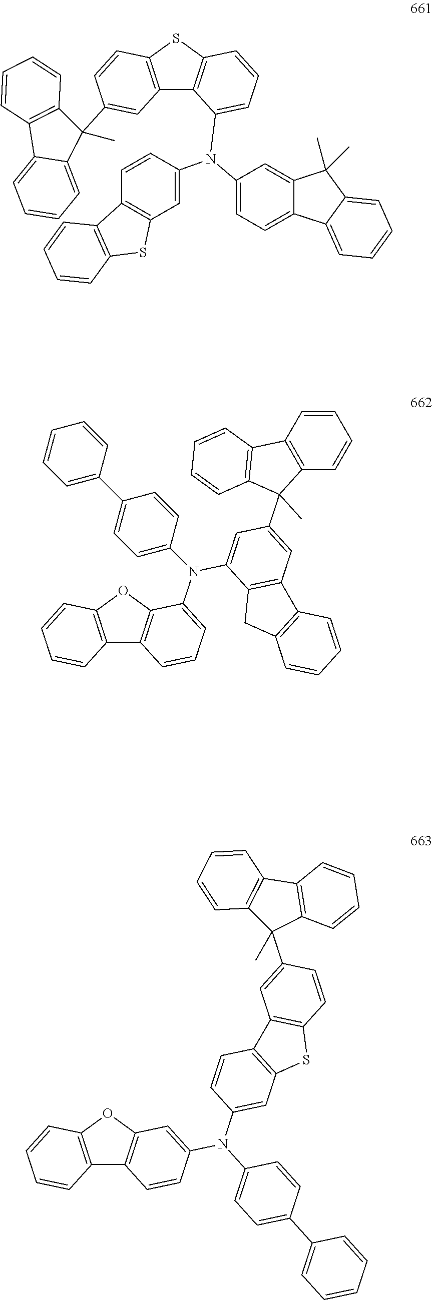 [SMILES: CC1(C)c2ccccc2-c2ccc(N(c3ccc4c(c3)sc3ccccc34)c3cccc4sc5ccc(C6(C)c7ccccc7-c7ccccc76)cc5c34)cc21 ; CC1(c2cc3c(c(N(c4ccc(-c5ccccc5)cc4)c4cccc5c4oc4ccccc45)c2)Cc2ccccc2-3)c2ccccc2-c2ccccc21 ; CC1(c2ccc3sc4cc(N(c5ccc(-c6ccccc6)cc5)c5ccc6c(c5)oc5ccccc56)ccc4c3c2)c2ccccc2-c2ccccc21]

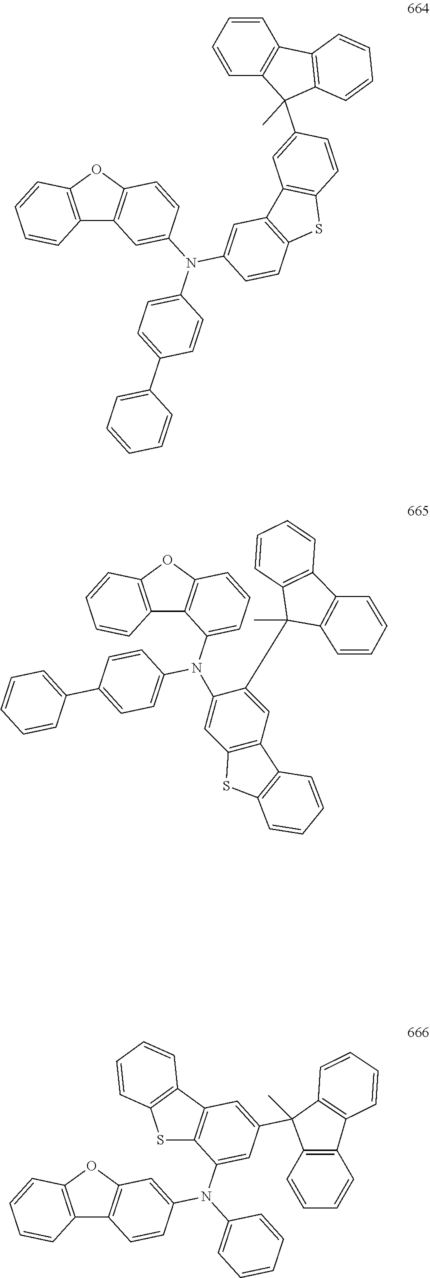 [SMILES: CC1(c2ccc3sc4ccc(N(c5ccc(-c6ccccc6)cc5)c5ccc6oc7ccccc7c6c5)cc4c3c2)c2ccccc2-c2ccccc21 ; CC1(c2cc3c(cc2N(c2ccc(-c4ccccc4)cc2)c2cccc4oc5ccccc5c24)sc2ccccc23)c2ccccc2-c2ccccc21 ; CC1(c2cc(N(c3ccccc3)c3ccc4c(c3)oc3ccccc34)c3sc4ccccc4c3c2)c2ccccc2-c2ccccc21]

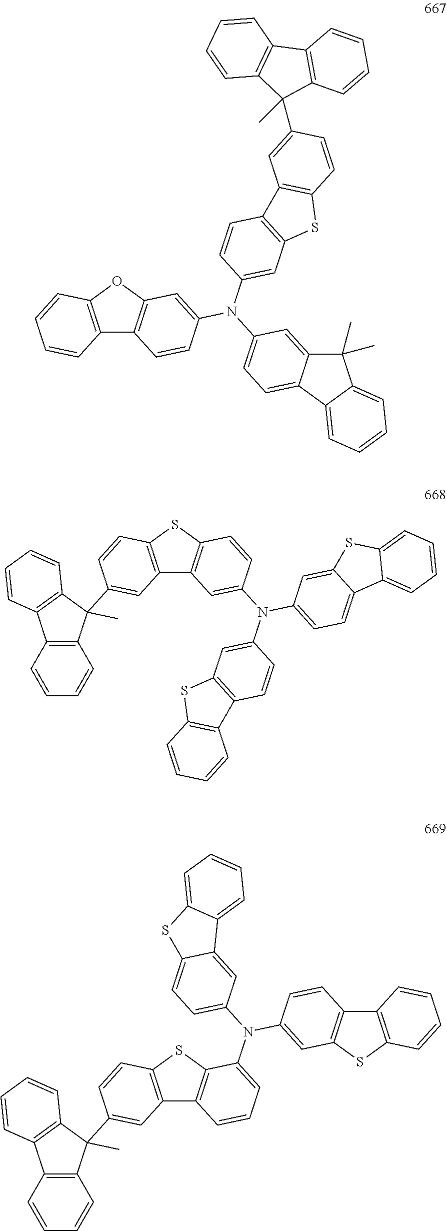 [SMILES: CC1(C)c2ccccc2-c2ccc(N(c3ccc4c(c3)oc3ccccc34)c3ccc4c(c3)sc3ccc(C5(C)c6ccccc6-c6ccccc65)cc34)cc21 ; CC1(c2ccc3sc4ccc(N(c5ccc6c(c5)sc5ccccc56)c5ccc6c(c5)sc5ccccc56)cc4c3c2)c2ccccc2-c2ccccc21 ; CC1(c2ccc3sc4c(N(c5ccc6c(c5)sc5ccccc56)c5ccc6sc7ccccc7c6c5)cccc4c3c2)c2ccccc2-c2ccccc21]

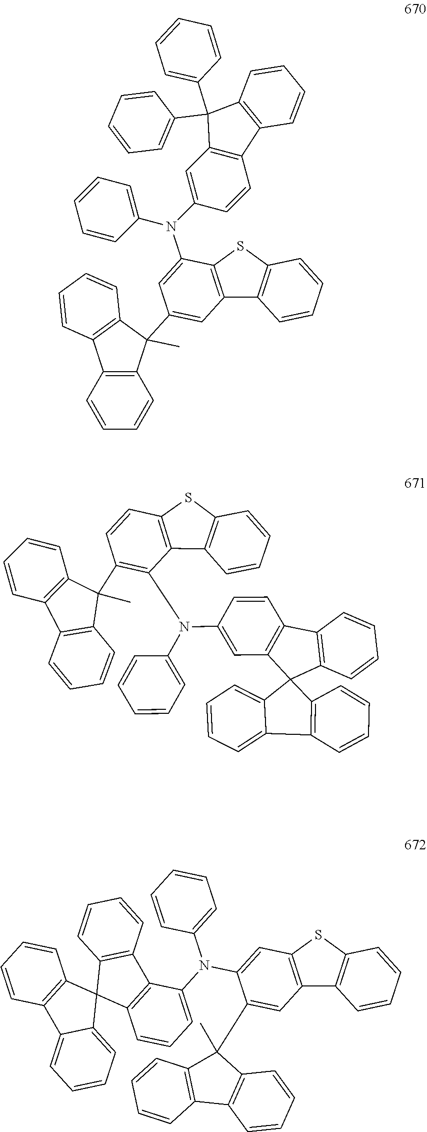 [SMILES: CC1(c2cc(N(c3ccccc3)c3ccc4c(c3)C(c3ccccc3)(c3ccccc3)c3ccccc3-4)c3sc4ccccc4c3c2)c2ccccc2-c2ccccc21 ; CC1(c2ccc3sc4ccccc4c3c2N(c2ccccc2)c2ccc3c(c2)C2(c4ccccc4-c4ccccc42)c2ccccc2-3)c2ccccc2-c2ccccc21 ; CC1(c2cc3c(cc2N(c2ccccc2)c2cccc4c2-c2ccccc2C42c4ccccc4-c4ccccc42)sc2ccccc23)c2ccccc2-c2ccccc21]

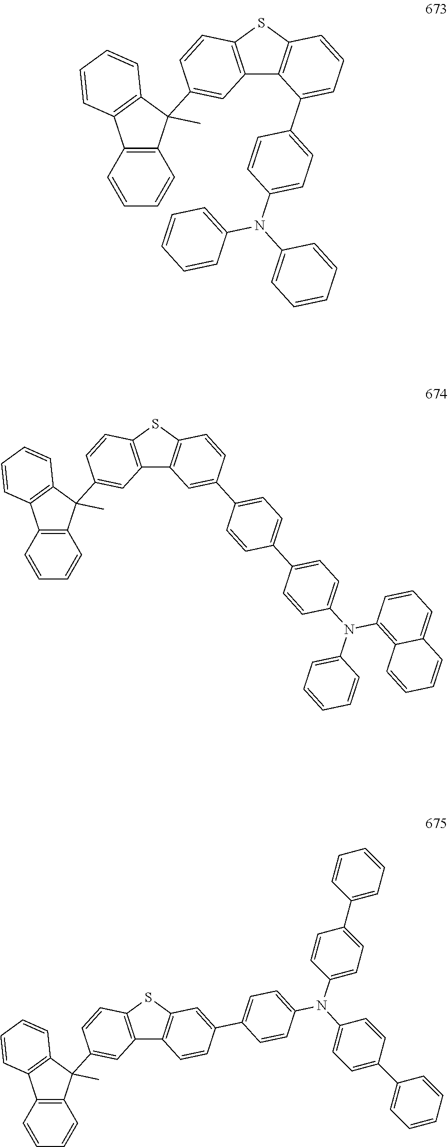 [SMILES: CC1(c2ccc3sc4cccc(-c5ccc(N(c6ccccc6)c6ccccc6)cc5)c4c3c2)c2ccccc2-c2ccccc21 ; CC1(c2ccc3sc4ccc(-c5ccc(-c6ccc(N(c7ccccc7)c7cccc8ccccc78)cc6)cc5)cc4c3c2)c2ccccc2-c2ccccc21 ; CC1(c2ccc3sc4cc(-c5ccc(N(c6ccc(-c7ccccc7)cc6)c6ccc(-c7ccccc7)cc6)cc5)ccc4c3c2)c2ccccc2-c2ccccc21]

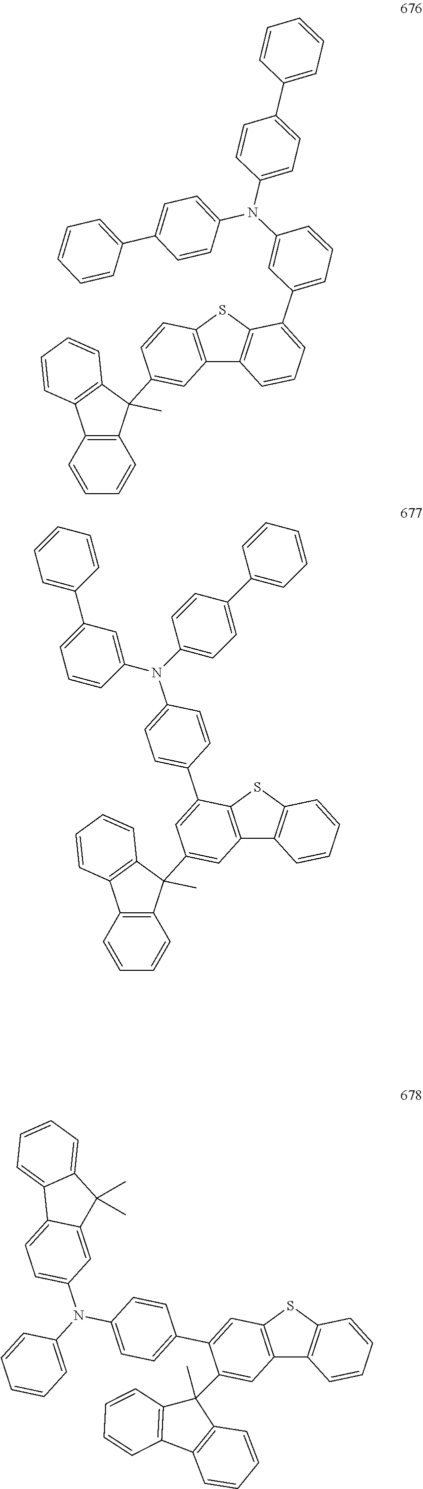

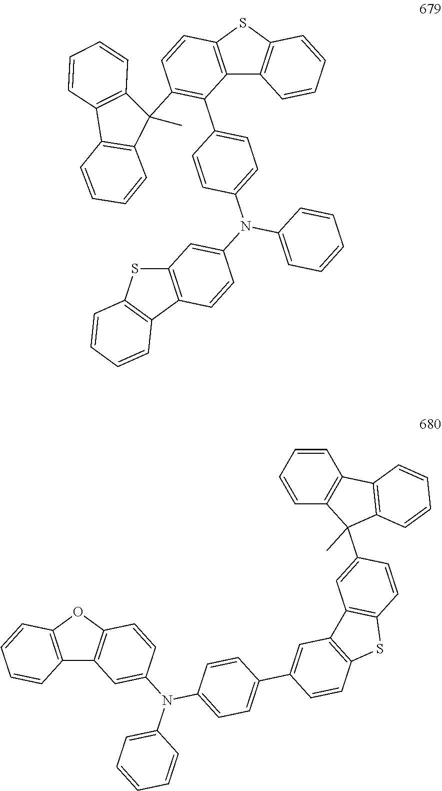 [SMILES: CC1(c2ccc3sc4ccccc4c3c2-c2ccc(N(c3ccccc3)c3ccc4c(c3)sc3ccccc34)cc2)c2ccccc2-c2ccccc21 ; CC1(c2ccc3sc4ccc(-c5ccc(N(c6ccccc6)c6ccc7oc8ccccc8c7c6)cc5)cc4c3c2)c2ccccc2-c2ccccc21]

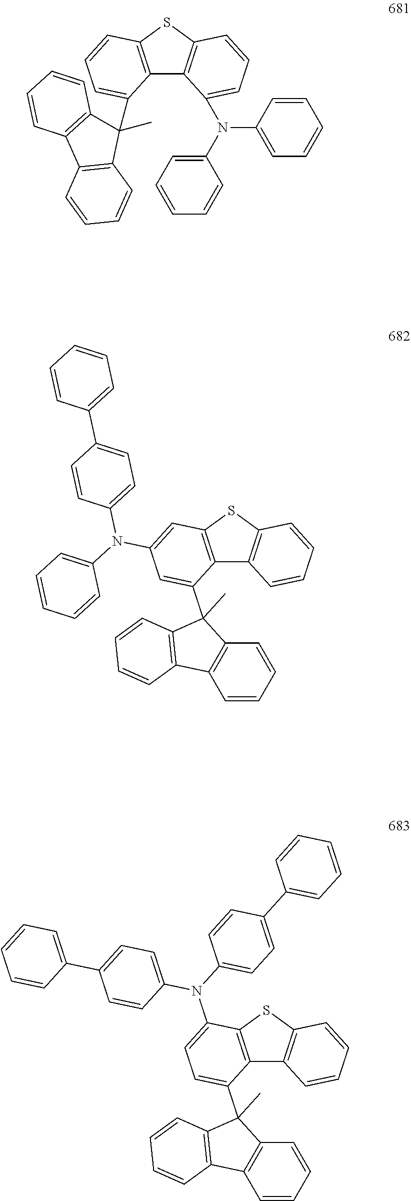 [SMILES: CC1(c2cccc3sc4cccc(N(c5ccccc5)c5ccccc5)c4c23)c2ccccc2-c2ccccc21 ; CC1(c2cc(N(c3ccccc3)c3ccc(-c4ccccc4)cc3)cc3sc4ccccc4c23)c2ccccc2-c2ccccc21 ; CC1(c2ccc(N(c3ccc(-c4ccccc4)cc3)c3ccc(-c4ccccc4)cc3)c3sc4ccccc4c23)c2ccccc2-c2ccccc21]

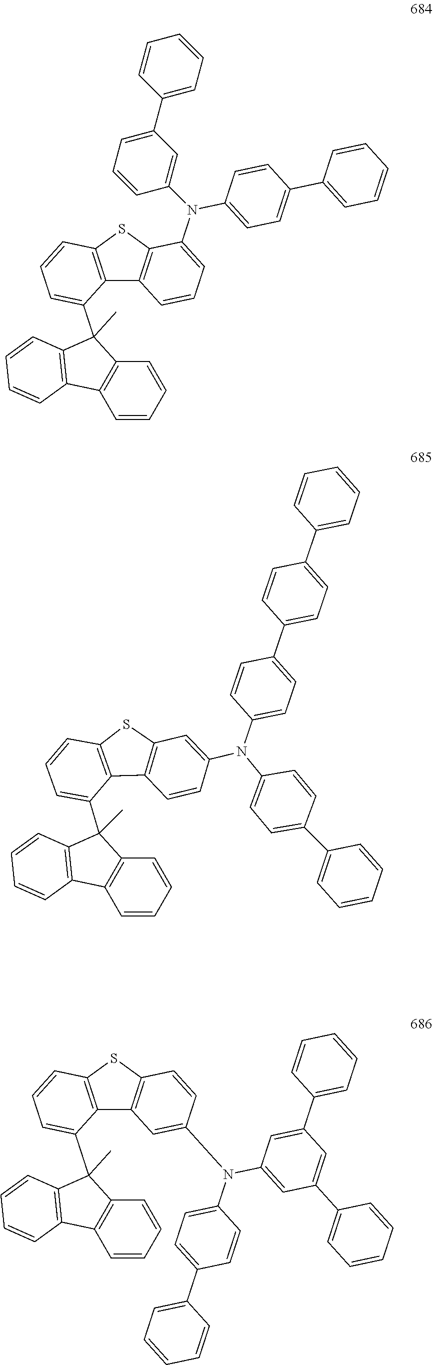 [SMILES: CC1(c2cccc3sc4c(N(c5ccc(-c6ccccc6)cc5)c5cccc(-c6ccccc6)c5)cccc4c23)c2ccccc2-c2ccccc21 ; CC1(c2cccc3sc4cc(N(c5ccc(-c6ccccc6)cc5)c5ccc(-c6ccc(-c7ccccc7)cc6)cc5)ccc4c23)c2ccccc2-c2ccccc21 ; CC1(c2cccc3sc4ccc(N(c5ccc(-c6ccccc6)cc5)c5cc(-c6ccccc6)cc(-c6ccccc6)c5)cc4c23)c2ccccc2-c2ccccc21]

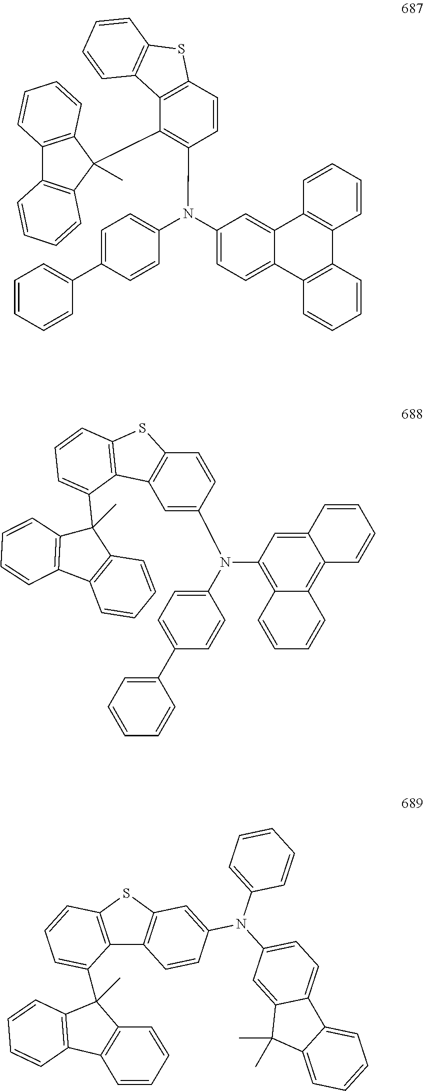 [SMILES: CC1(c2c(N(c3ccc(-c4ccccc4)cc3)c3ccc4c5ccccc5c5ccccc5c4c3)ccc3sc4ccccc4c23)c2ccccc2-c2ccccc21 ; CC1(c2cccc3sc4ccc(N(c5ccc(-c6ccccc6)cc5)c5cc6ccccc6c6ccccc56)cc4c23)c2ccccc2-c2ccccc21 ; CC1(C)c2ccccc2-c2ccc(N(c3ccccc3)c3ccc4c(c3)sc3cccc(C5(C)c6ccccc6-c6ccccc65)c34)cc21]

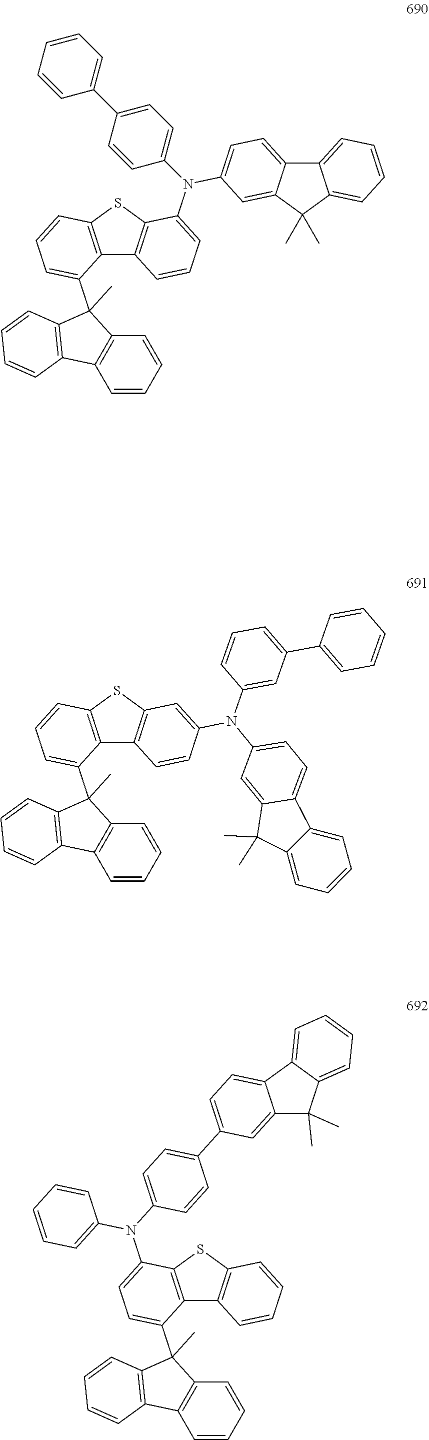 [SMILES: CC1(C)c2ccccc2-c2ccc(N(c3ccc(-c4ccccc4)cc3)c3cccc4c3sc3cccc(C5(C)c6ccccc6-c6ccccc65)c34)cc21 ; CC1(C)c2ccccc2-c2ccc(N(c3cccc(-c4ccccc4)c3)c3ccc4c(c3)sc3cccc(C5(C)c6ccccc6-c6ccccc65)c34)cc21 ; CC1(C)c2ccccc2-c2ccc(-c3ccc(N(c4ccccc4)c4ccc(C5(C)c6ccccc6-c6ccccc65)c5c4sc4ccccc45)cc3)cc21]

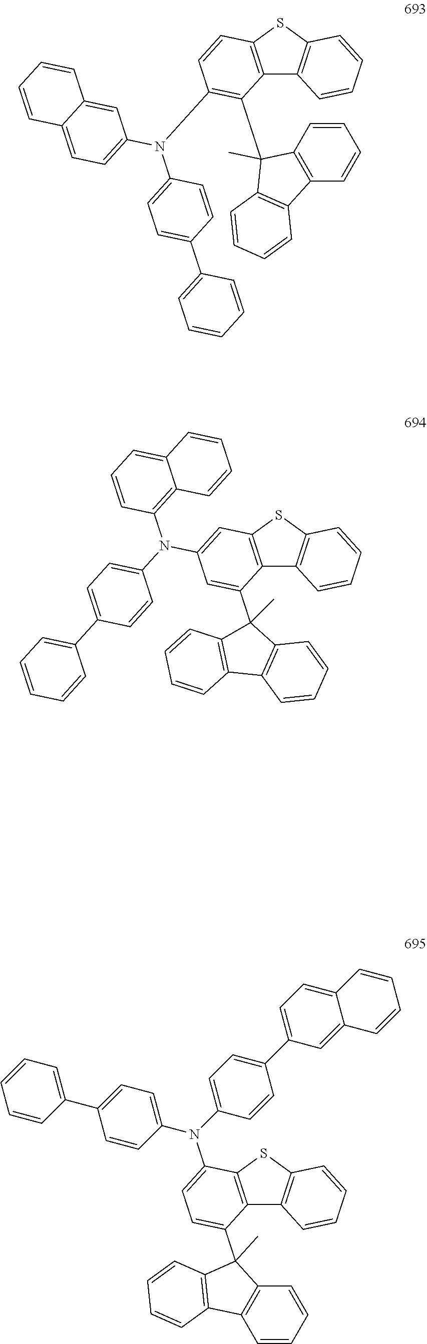 [SMILES: CC1(c2c(N(c3ccc(-c4ccccc4)cc3)c3ccc4ccccc4c3)ccc3sc4ccccc4c23)c2ccccc2-c2ccccc21 ; CC1(c2cc(N(c3ccc(-c4ccccc4)cc3)c3cccc4ccccc34)cc3sc4ccccc4c23)c2ccccc2-c2ccccc21 ; CC1(c2ccc(N(c3ccc(-c4ccccc4)cc3)c3ccc(-c4ccc5ccccc5c4)cc3)c3sc4ccccc4c23)c2ccccc2-c2ccccc21]

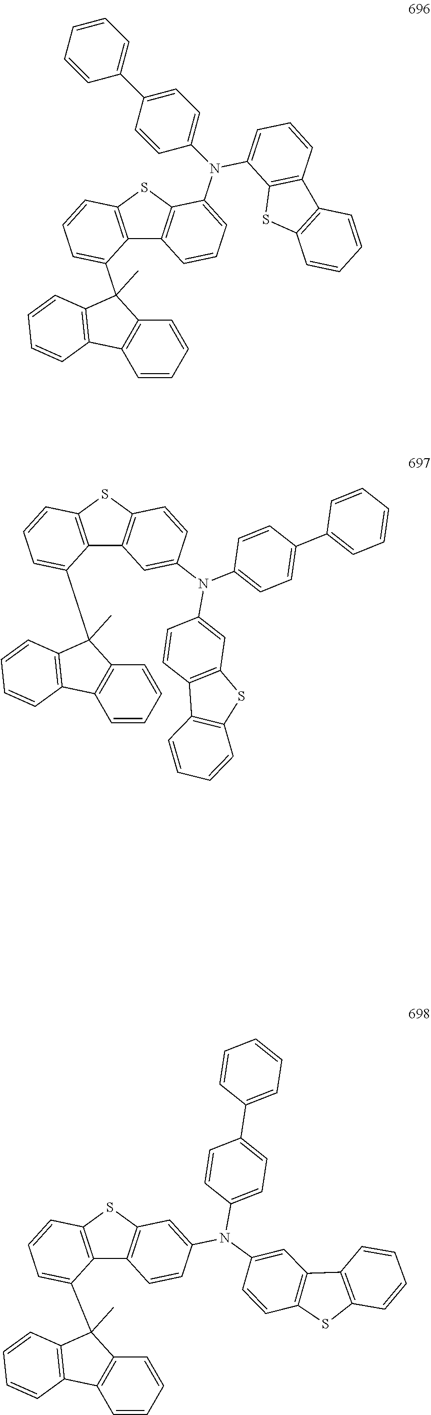 [SMILES: CC1(c2cccc3sc4c(N(c5ccc(-c6ccccc6)cc5)c5cccc6c5sc5ccccc56)cccc4c23)c2ccccc2-c2ccccc21 ; CC1(c2cccc3sc4ccc(N(c5ccc(-c6ccccc6)cc5)c5ccc6c(c5)sc5ccccc56)cc4c23)c2ccccc2-c2ccccc21 ; CC1(c2cccc3sc4cc(N(c5ccc(-c6ccccc6)cc5)c5ccc6sc7ccccc7c6c5)ccc4c23)c2ccccc2-c2ccccc21]

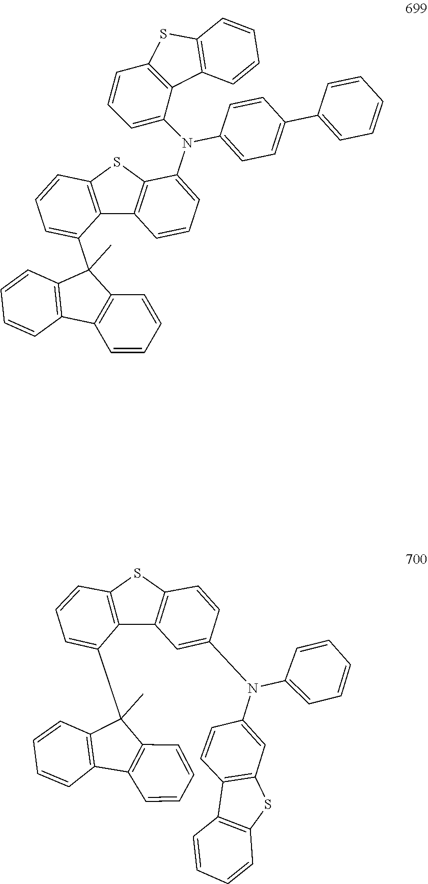 [SMILES: CC1(c2cccc3sc4c(N(c5ccc(-c6ccccc6)cc5)c5cccc6sc7ccccc7c56)cccc4c23)c2ccccc2-c2ccccc21 ; CC1(c2cccc3sc4ccc(N(c5ccccc5)c5ccc6c(c5)sc5ccccc56)cc4c23)c2ccccc2-c2ccccc21]

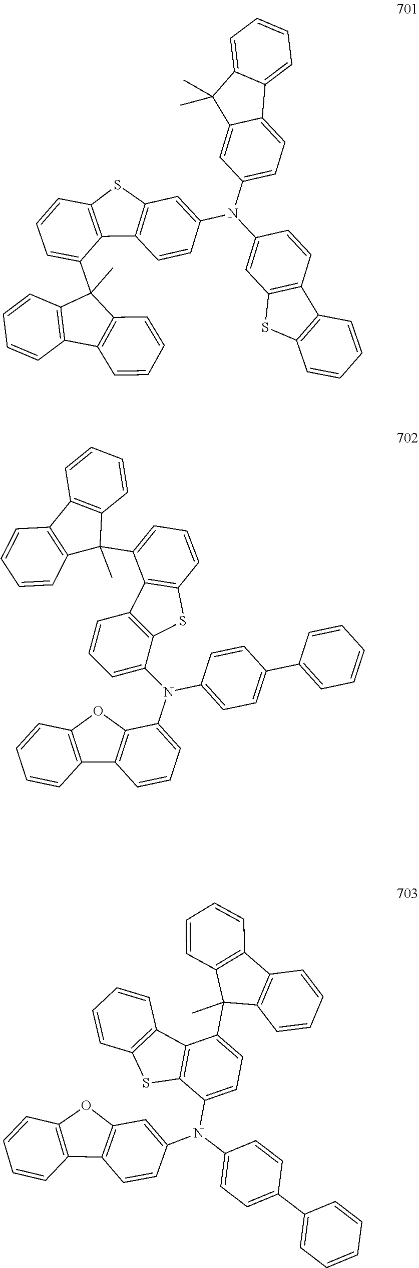 [SMILES: CC1(C)c2ccccc2-c2ccc(N(c3ccc4c(c3)sc3ccccc34)c3ccc4c(c3)sc3cccc(C5(C)c6ccccc6-c6ccccc65)c34)cc21 ; CC1(c2cccc3sc4c(N(c5ccc(-c6ccccc6)cc5)c5cccc6c5oc5ccccc56)cccc4c23)c2ccccc2-c2ccccc21 ; CC1(c2ccc(N(c3ccc(-c4ccccc4)cc3)c3ccc4c(c3)oc3ccccc34)c3sc4ccccc4c23)c2ccccc2-c2ccccc21]

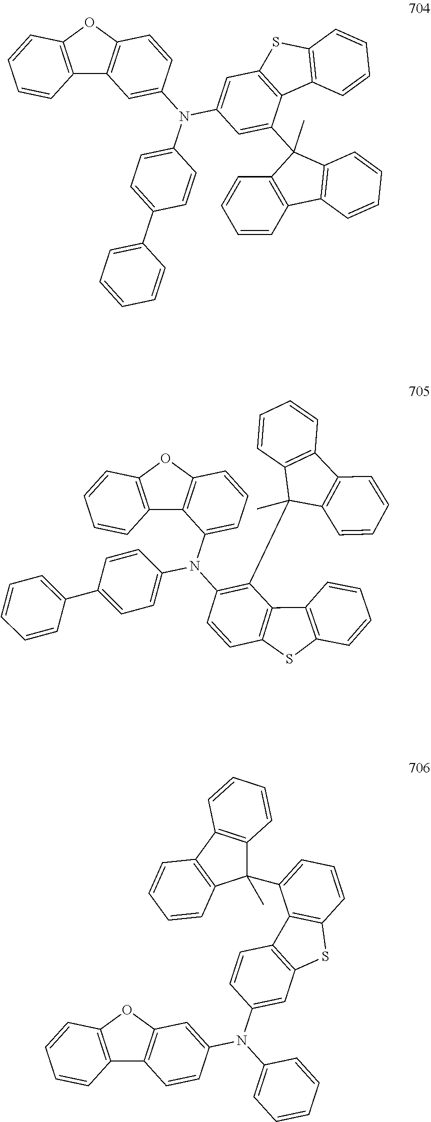 [SMILES: CC1(c2cc(N(c3ccc(-c4ccccc4)cc3)c3ccc4oc5ccccc5c4c3)cc3sc4ccccc4c23)c2ccccc2-c2ccccc21 ; CC1(c2c(N(c3ccc(-c4ccccc4)cc3)c3cccc4oc5ccccc5c34)ccc3sc4ccccc4c23)c2ccccc2-c2ccccc21 ; CC1(c2cccc3sc4cc(N(c5ccccc5)c5ccc6c(c5)oc5ccccc56)ccc4c23)c2ccccc2-c2ccccc21]

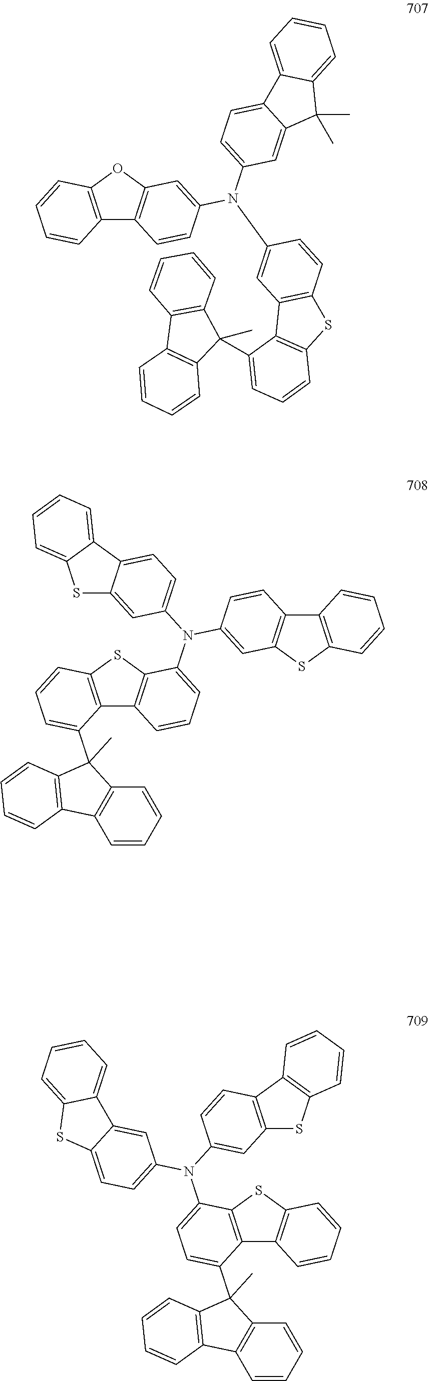 [SMILES: CC1(C)c2ccccc2-c2ccc(N(c3ccc4c(c3)oc3ccccc34)c3ccc4sc5cccc(C6(C)c7ccccc7-c7ccccc76)c5c4c3)cc21 ; CC1(c2cccc3sc4c(N(c5ccc6c(c5)sc5ccccc56)c5ccc6c(c5)sc5ccccc56)cccc4c23)c2ccccc2-c2ccccc21 ; CC1(c2ccc(N(c3ccc4c(c3)sc3ccccc34)c3ccc4sc5ccccc5c4c3)c3sc4ccccc4c23)c2ccccc2-c2ccccc21]

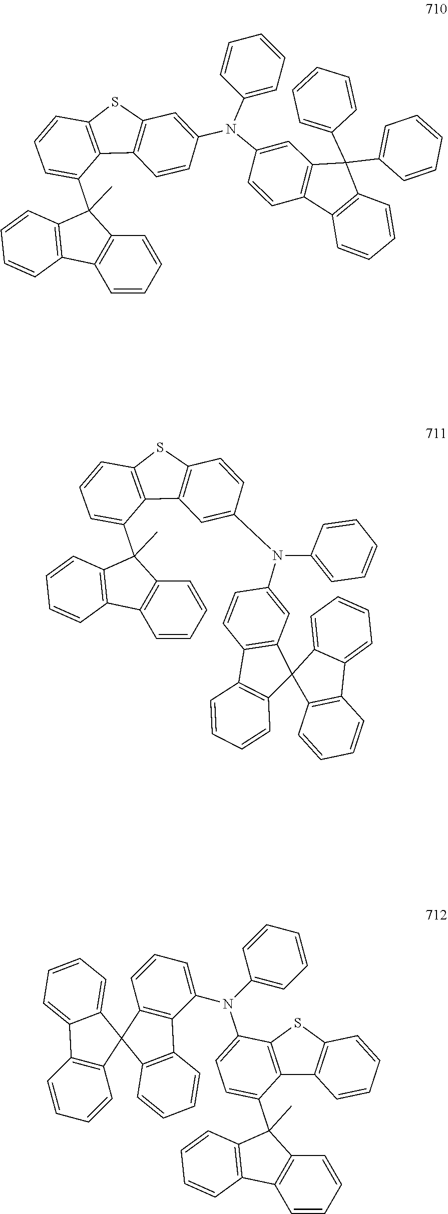 [SMILES: CC1(c2cccc3sc4cc(N(c5ccccc5)c5ccc6c(c5)C(c5ccccc5)(c5ccccc5)c5ccccc5-6)ccc4c23)c2ccccc2-c2ccccc21 ; CC1(c2cccc3sc4ccc(N(c5ccccc5)c5ccc6c(c5)C5(c7ccccc7-c7ccccc75)c5ccccc5-6)cc4c23)c2ccccc2-c2ccccc21 ; CC1(c2ccc(N(c3ccccc3)c3cccc4c3-c3ccccc3C43c4ccccc4-c4ccccc43)c3sc4ccccc4c23)c2ccccc2-c2ccccc21]

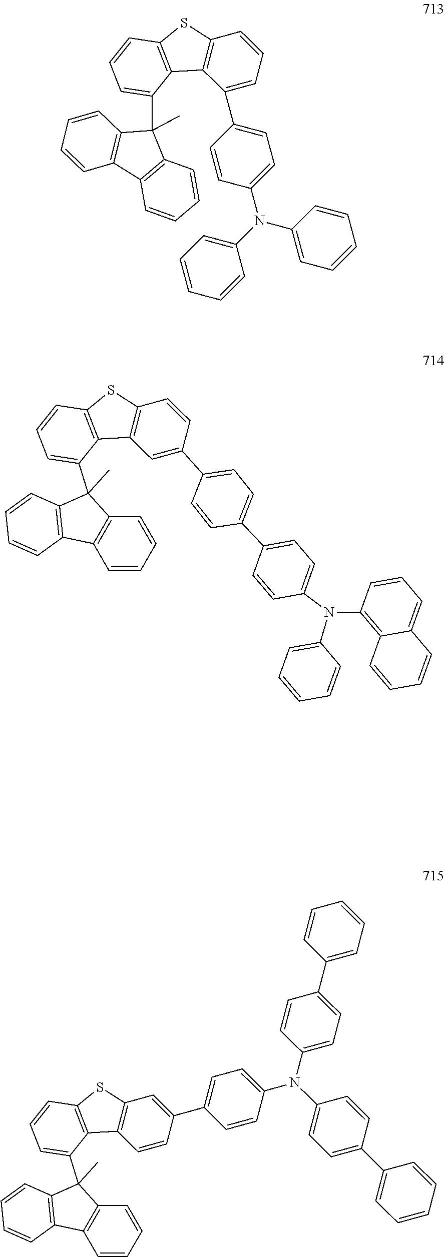 [SMILES: CC1(c2cccc3sc4cccc(-c5ccc(N(c6ccccc6)c6ccccc6)cc5)c4c23)c2ccccc2-c2ccccc21 ; CC1(c2cccc3sc4ccc(-c5ccc(-c6ccc(N(c7ccccc7)c7cccc8ccccc78)cc6)cc5)cc4c23)c2ccccc2-c2ccccc21 ; CC1(c2cccc3sc4cc(-c5ccc(N(c6ccc(-c7ccccc7)cc6)c6ccc(-c7ccccc7)cc6)cc5)ccc4c23)c2ccccc2-c2ccccc21]

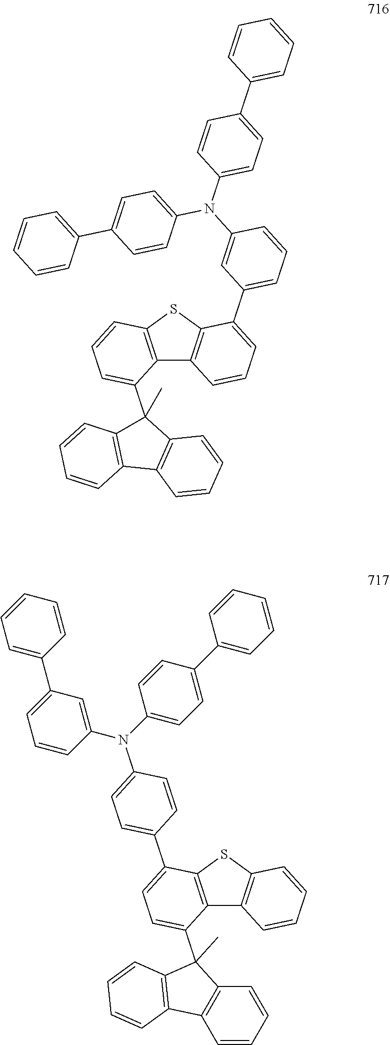 [SMILES: CC1(c2cccc3sc4c(-c5cccc(N(c6ccc(-c7ccccc7)cc6)c6ccc(-c7ccccc7)cc6)c5)cccc4c23)c2ccccc2-c2ccccc21 ; CC1(c2ccc(-c3ccc(N(c4ccc(-c5ccccc5)cc4)c4cccc(-c5ccccc5)c4)cc3)c3sc4ccccc4c23)c2ccccc2-c2ccccc21]

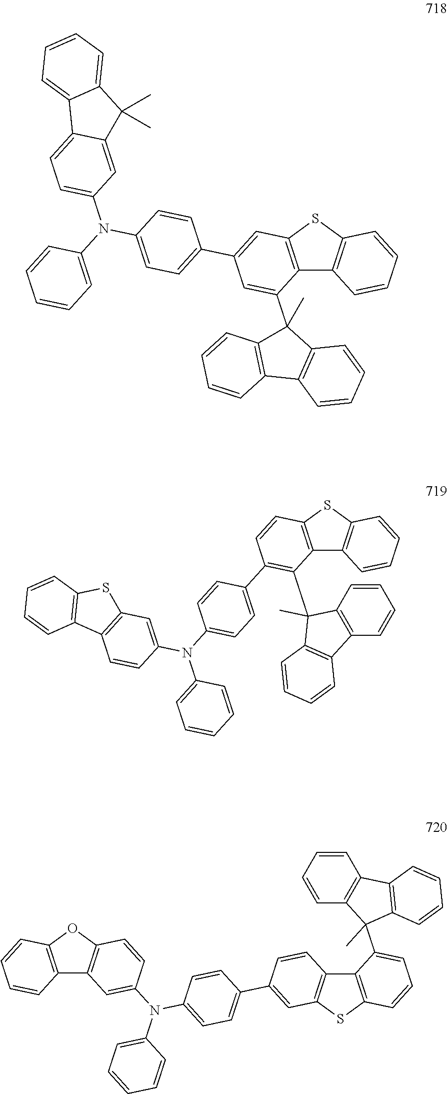 [SMILES: CC1(C)c2ccccc2-c2ccc(N(c3ccccc3)c3ccc(-c4cc(C5(C)c6ccccc6-c6ccccc65)c5c(c4)sc4ccccc45)cc3)cc21 ; CC1(c2c(-c3ccc(N(c4ccccc4)c4ccc5c(c4)sc4ccccc45)cc3)ccc3sc4ccccc4c23)c2ccccc2-c2ccccc21 ; CC1(c2cccc3sc4cc(-c5ccc(N(c6ccccc6)c6ccc7oc8ccccc8c7c6)cc5)ccc4c23)c2ccccc2-c2ccccc21]

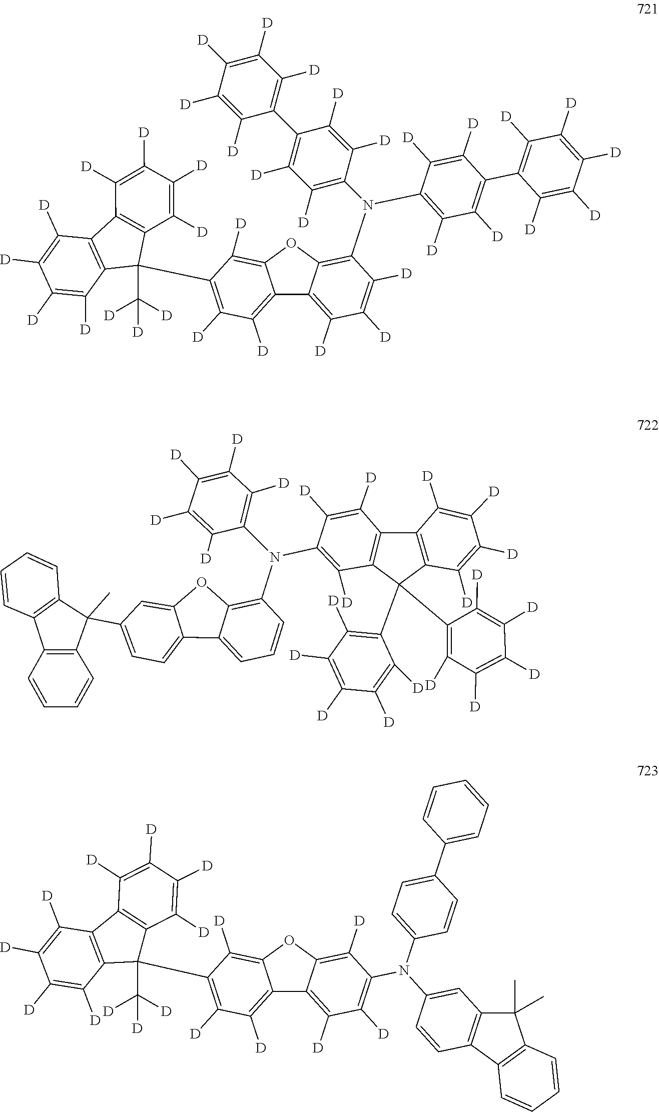

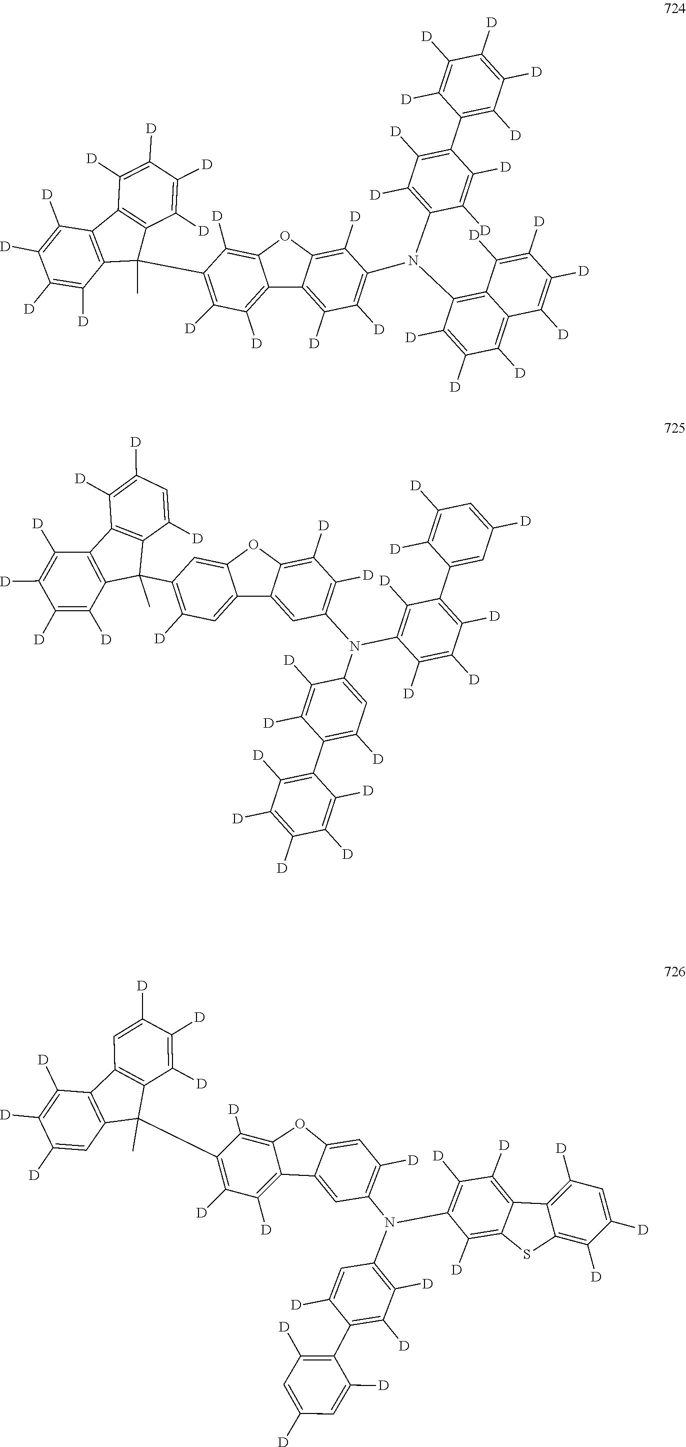

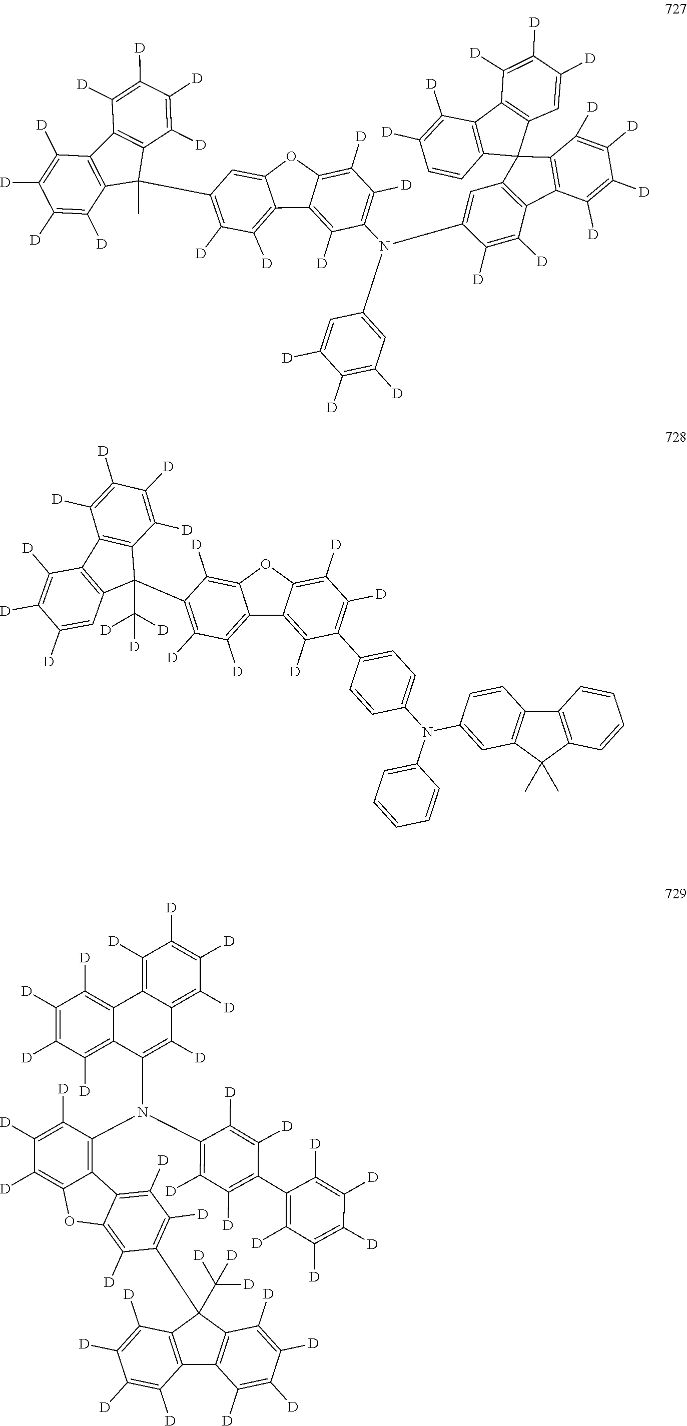

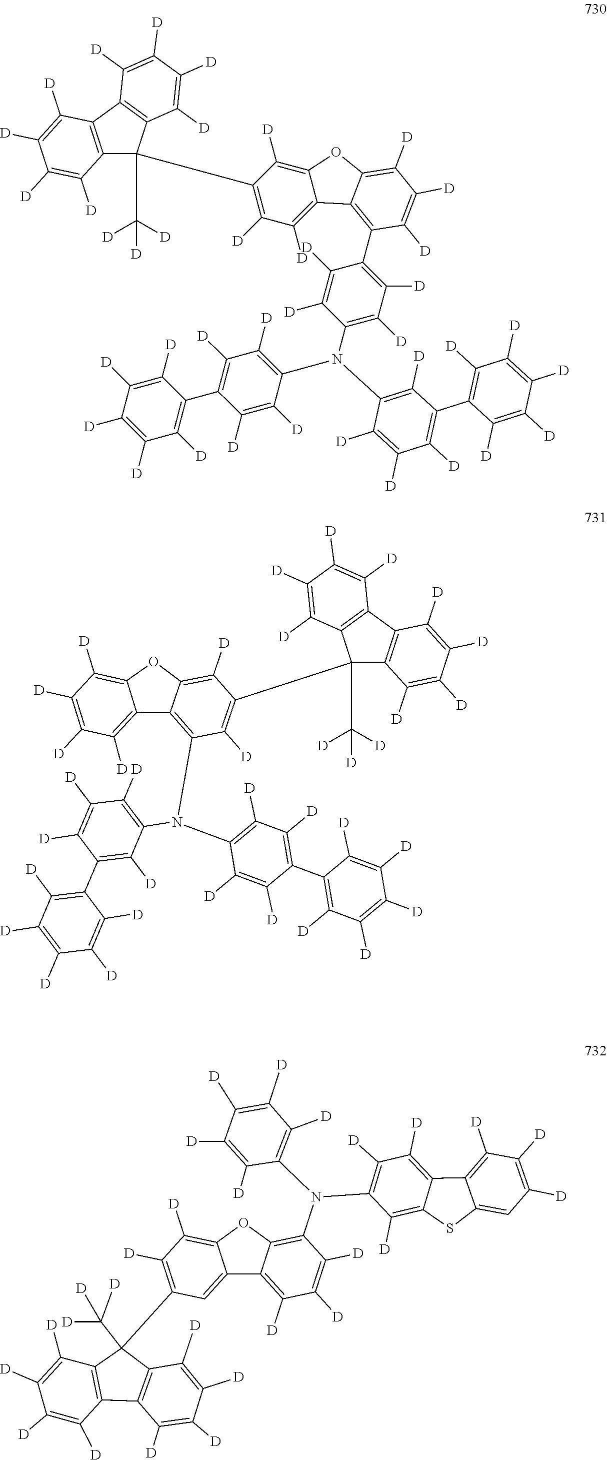

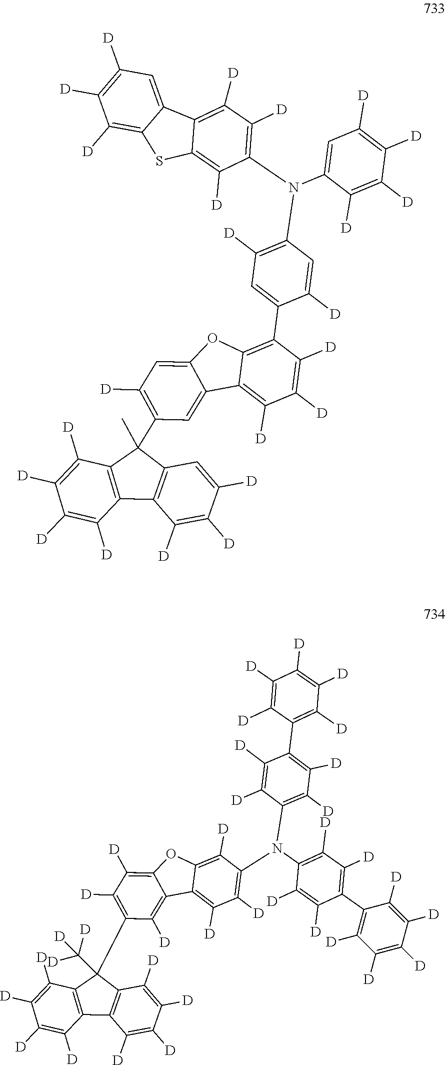

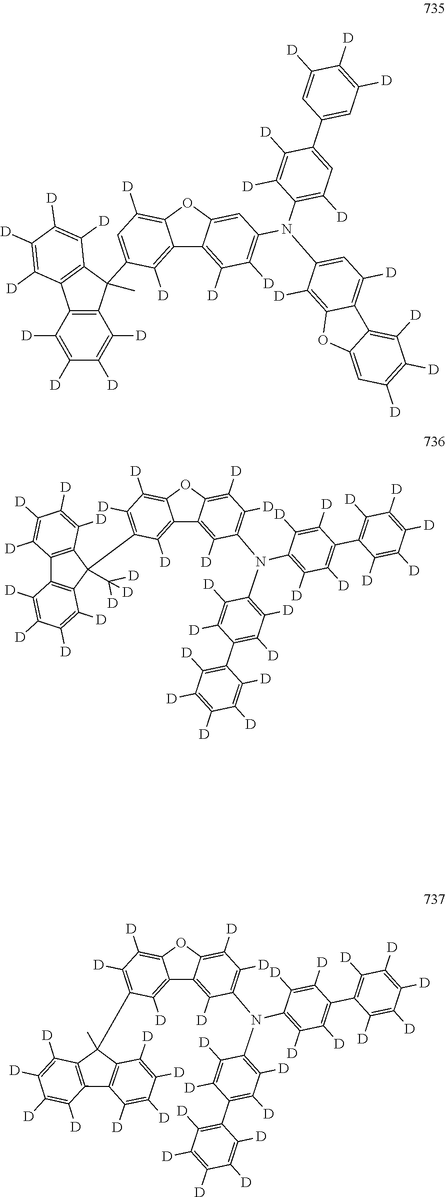

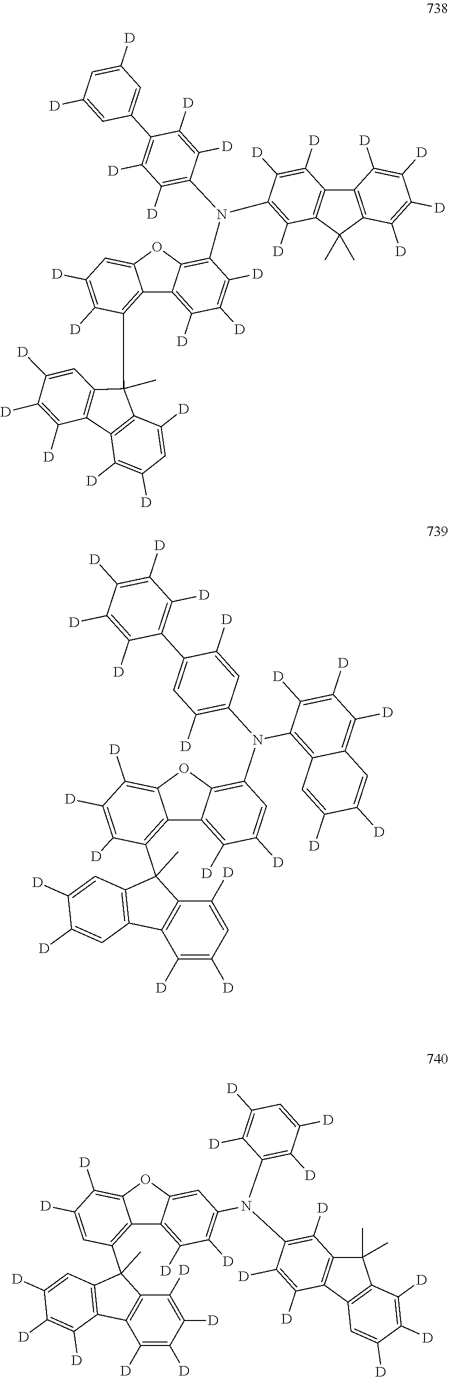

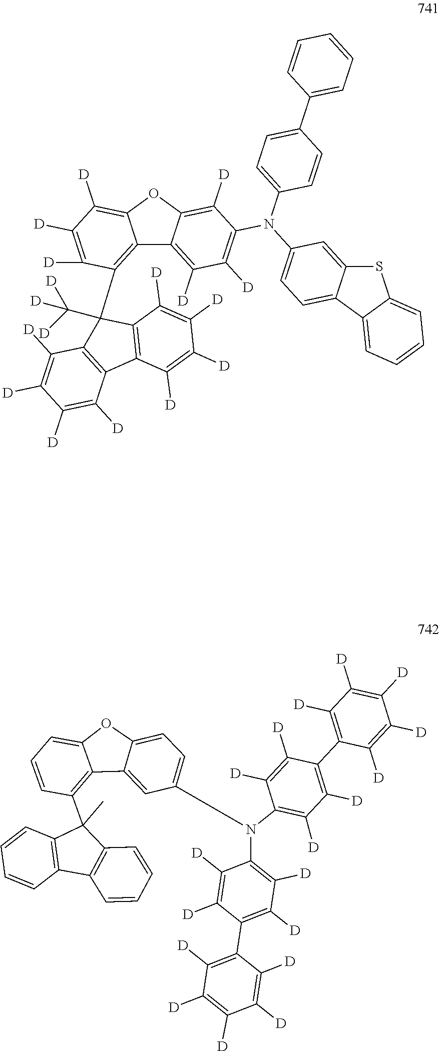 [SMILES: [2H]c1c([2H])c([2H])c2c(c1[2H])-c1c([2H])c([2H])c([2H])c([2H])c1C2(c1c([2H])c([2H])c([2H])c2oc3c([2H])c(N(c4ccc(-c5ccccc5)cc4)c4ccc5c(c4)sc4ccccc45)c([2H])c([2H])c3c12)C([2H])([2H])[2H] ; [2H]c1c([2H])c([2H])c(-c2c([2H])c([2H])c(N(c3ccc4oc5cccc(C6(C)c7ccccc7-c7ccccc76)c5c4c3)c3c([2H])c([2H])c(-c4c([2H])c([2H])c([2H])c([2H])c4[2H])c([2H])c3[2H])c([2H])c2[2H])c([2H])c1[2H]]

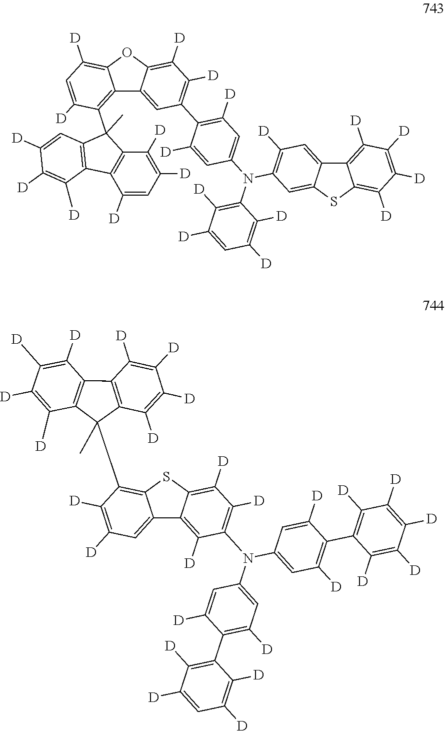 [SMILES: [2H]c1cc2c(cc1N(c1cc([2H])c(-c3cc4c(oc5c([2H])cc([2H])c(C6(C)c7cc([2H])c([2H])c([2H])c7-c7c([2H])cc([2H])c([2H])c76)c54)c([2H])c3[2H])c([2H])c1)c1c([2H])c([2H])cc([2H])c1[2H])sc1c([2H])c([2H])c([2H])c([2H])c12 ; [2H]c1cc([2H])c([2H])c(-c2c([2H])cc(N(c3cc([2H])c(-c4c([2H])c([2H])c([2H])c([2H])c4[2H])c([2H])c3)c3c([2H])c([2H])c4sc5c(C6(C)c7c([2H])c([2H])c([2H])c([2H])c7-c7c([2H])c([2H])c([2H])c([2H])c76)c([2H])c([2H])cc5c4c3[2H])cc2[2H])c1[2H]]

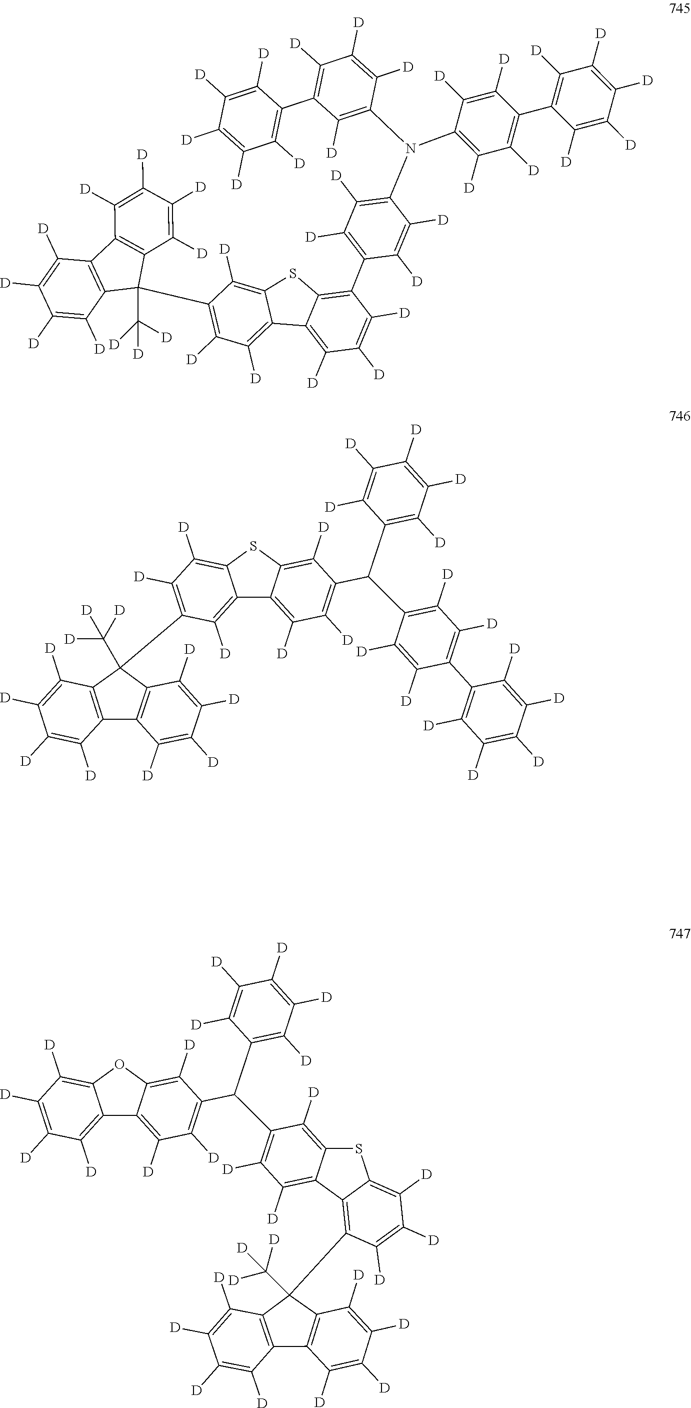

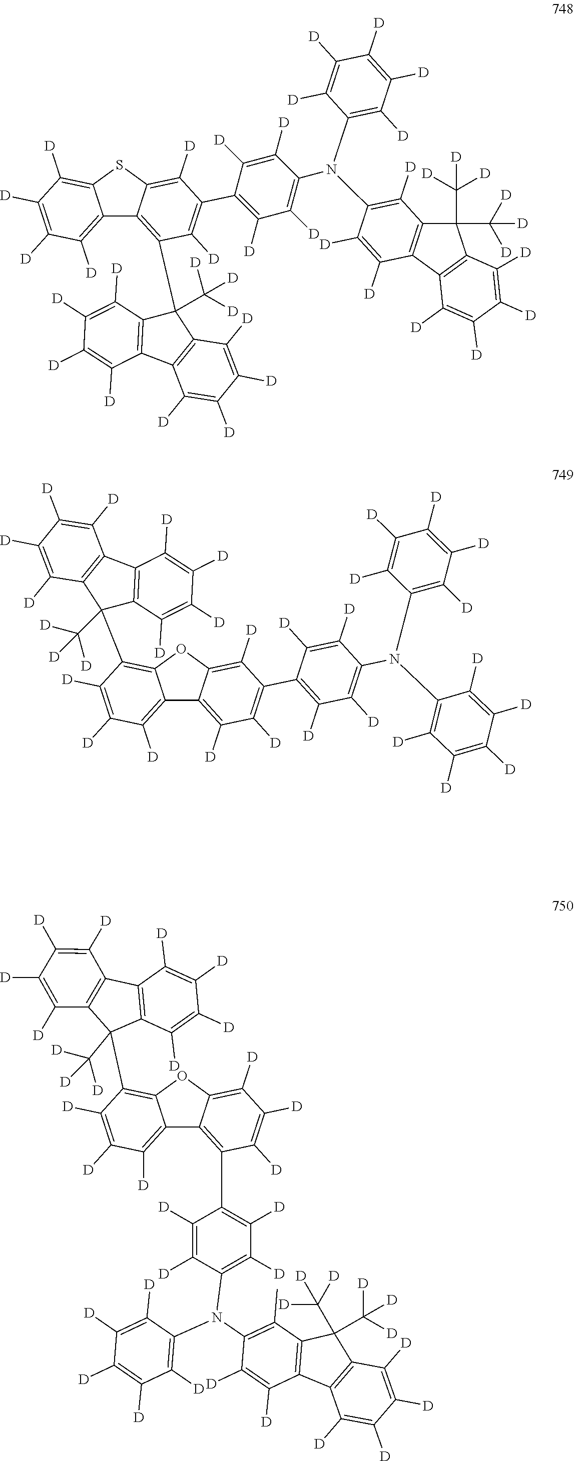

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a material for transporting holes, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

Meanwhile, the heterocyclic compound has a high glass transition temperature (Tg) and thus has excellent thermal stability. The increase in thermal stability becomes an important factor for providing a device with driving stability.

The heterocyclic compound according to an exemplary embodiment of the present application may be prepared by a multi-step chemical reaction. Some intermediate compounds are first prepared, and the compound of Chemical Formula 1 may be prepared from the intermediate compounds. More specifically, the heterocyclic compound according to an exemplary embodiment of the present application may be prepared based on Preparation Examples to be described below.

Another exemplary embodiment of the present application provides an organic light emitting device including the heterocyclic compound represented by Chemical Formula 1. The “organic light emitting device” may be expressed by terms such as “organic light emitting diode”, “organic light emitting diodes (OLEDs)”, “OLED device”, and “organic electroluminescence device”.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

Specifically, the organic light emitting device according to an exemplary embodiment of the present application includes a first electrode, a second electrode and an organic material layer having one or more layers provided between the first electrode and the second electrode, in which one or more layers of the organic material layer include the heterocyclic compound represented by Chemical Formula 1. When the heterocyclic compound represented by Chemical Formula 1 is included in the organic material layer, the light emitting efficiency and service life of the organic light emitting device are excellent.

In an exemplary embodiment of the present application, the first electrode may be a negative electrode, and the second electrode may be a positive electrode.

In an exemplary embodiment of the present application, the first electrode may be a positive electrode, and the second electrode may be a negative electrode.

Further, the organic material layer includes a hole transport layer, and the hole transport layer may include the heterocyclic compound represented by Chemical Formula 1. When the heterocyclic compound represented by Chemical Formula 1 is included in the hole transport layer among the organic material layers, the light emitting efficiency and service life of the organic light emitting device are better.

In the organic light emitting device of the present application, the organic material layer includes an electron blocking layer, and the electron blocking layer may include the heterocyclic compound. When the heterocyclic compound represented by Chemical Formula 1 is included in the electron blocking layer among the organic material layers, the light emitting efficiency and service life of the organic light emitting device are better.

In the organic light emitting device of the present application, the organic material layer includes a hole transport auxiliary layer, and the hole transport auxiliary layer may include the heterocyclic compound. When the heterocyclic compound represented by Chemical Formula 1 is included in the hole transport auxiliary layer among the organic material layers, the light emitting efficiency and service life of the organic light emitting device are better.

In addition, the organic material layer includes a light emitting layer, and the light emitting layer includes the heterocyclic compound represented by Chemical Formula 1. When the heterocyclic compound represented by Chemical Formula 1 is included in the light emitting layer among the organic material layers, the light emitting efficiency and service life of the organic light emitting device are better.

In the organic light emitting device of the present application, the light emitting layer may include a host material, and the host material may include the heterocyclic compound represented by Chemical Formula 1.

In the organic light emitting device of the present application, the light emitting layer may include two or more host materials. One or more of the host materials may include the heterocyclic compound represented by Chemical Formula 1.

In the organic light emitting device of the present application, the light emitting layer may include two or more host materials, and the two or more host materials may each include one or more p-type host materials and n-type host materials.

In the organic light emitting device of the present application, the light emitting layer may be used by pre-mixing two or more host materials. The pre-mixing means that for the light emitting layer, before two or more host materials are deposited onto an organic material layer, the materials are first mixed and the mixture is contained in one common container and mixed.

The organic light emitting device according to an exemplary embodiment of the present application may be manufactured by typical manufacturing methods and materials of the organic light emitting device, except that the above-described heterocyclic compound is used to form an organic material layer.

The organic light emitting device of the present invention may further include one or two or more layers selected from the group consisting of a light emitting layer, a hole injection layer, a hole transport layer, an electron blocking layer, an electron injection layer, an electron transport layer, a hole auxiliary layer, and a hole blocking layer.

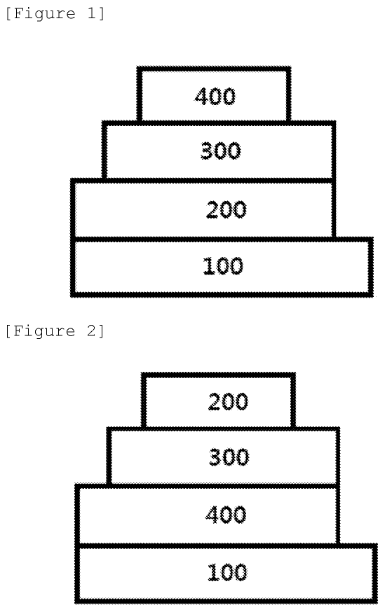

FIGS. 1 to 3 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

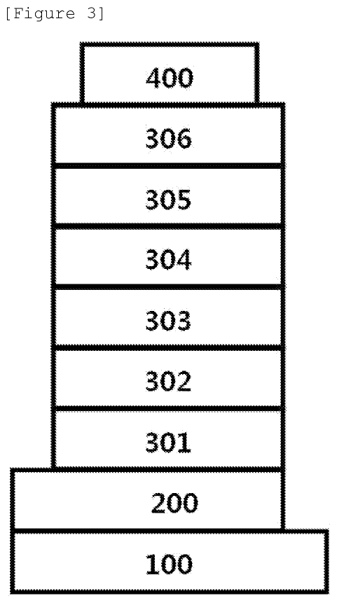

FIG. 3 exemplifies a case where an organic material layer is a multilayer. An organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

In the organic light emitting device according to an exemplary embodiment of the present application, materials other than the heterocyclic compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as Zno:Al or SnO₂:Sb; a conductive polymer such as poly(3-methylthiophene), poly[3,4-(ethylene-1,2-dioxy)thiophene](PEDT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like. As a hole transport material, a pyrazoline derivative, an arylamine-based derivative, a stilbene derivative, a triphenyldiamine derivative, and the like may be used, and a low-molecular weight or polymer material may also be used. As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices including organic solar cells, organic photoconductors, organic transistors, and the like, based on the principle similar to those applied to organic light emitting devices.

Hereinafter, the present specification will be described in more detail through Examples, but these Examples are provided only for exemplifying the present application, and are not intended to limit the scope of the present application.

### <Preparation Example 1> Preparation of Compound 003

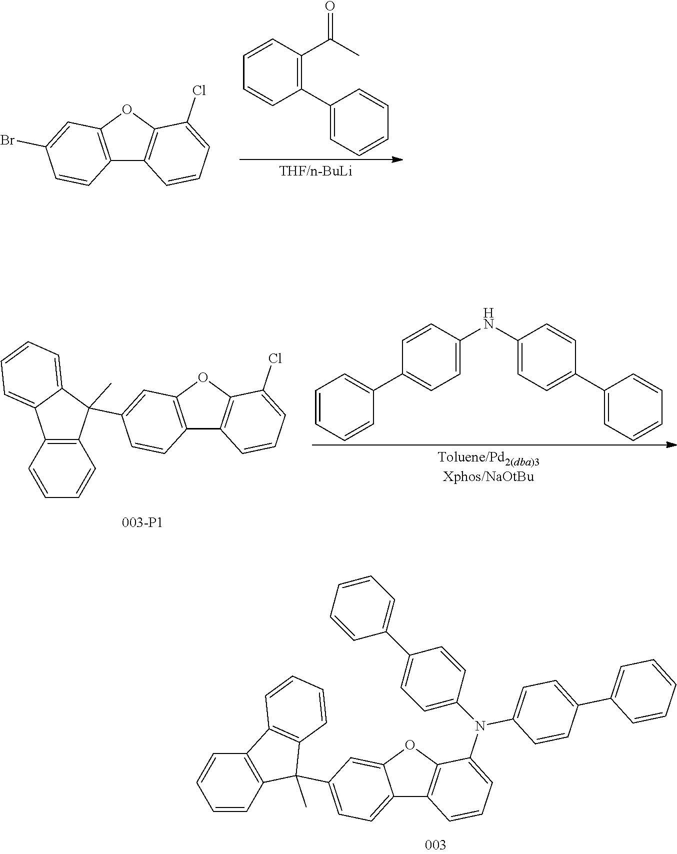

### 1) Preparation of Compound 003-P1

3-bromo-6-chlorodibenzo[b,d]furan (50 g, 177.60 mmol) was put into 500 ml of anhydrous tetrahydrofuran (hereinafter, THF) and the resulting mixture was purged with N₂. After the temperature of the reactor was lowered to −78° C., n-butyl lithium (n-BuLi) (78.1 ml, 2.5 M in hexane) was added dropwise and introduced into the reactor. After the mixture was stirred at the same temperature for 30 minutes, 1-([1,1′-biphenyl]-2-yl)ethanone (34.85 g, 177.60 mmol) was added thereto, and then the resulting mixture was stirred at room temperature for 12 hours (h). After the reaction was completed, extraction with ethyl acetate (hereinafter, EA) and distilled water (H₂O) was performed, the solvent was removed from the organic layer by a rotary evaporator, and then 500 ml of acetic acid and 50 ml of hydrochloric acid (HCl) were added thereto, and the resulting mixture was stirred under reflux for 3 hours. After the reaction was completed, dichloromethane was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 003-P1 (50 g, 74%).

### 2) Preparation of Compound 003

After Compound 003-P1 (10 g, 26.26 mmol) and di([1,1′-biphenyl]-4-yl)amine (8.86 g, 27.57 mmol) were dissolved in 100 ml of toluene, and then Pd₂(dba)₃ (1.20 g, 1.31 mmol), Xphos (1.25 g, 2.63 mmol), and sodium tert-butoxide (BuONa) (5.05 g, 52.51 mmol) were added thereto, and the resulting mixture was stirred under reflux for 2 hours. After the reaction was completed, dichloromethane was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 003 (14 g, 80%).

Further, a target compound in the following Table 1 was synthesized in the same manner as in the preparation method in Preparation Example 1, except that Compound A in the following Table 1 was used instead of 3-bromo-6-chlorodibenzo[b,d]furan and Compound B in the following Table 1 was used instead of di([1,1′-biphenyl]-4-yl)amine, in Preparation Example 1.

TABLE 1

| Com- | | | | |
|---|---|---|---|---|
| pound | | | | |
| No. | Compound A | Compound B | Target compound | yield |
| 010 | 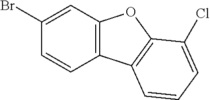 [SMILES: Clc1cccc2c1oc1cc(Br)ccc12] | 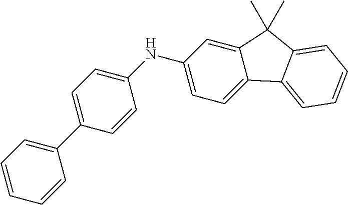 [SMILES: CC1(C)c2ccccc2-c2ccc(Nc3ccc(-c4ccccc4)cc3)cc21] | 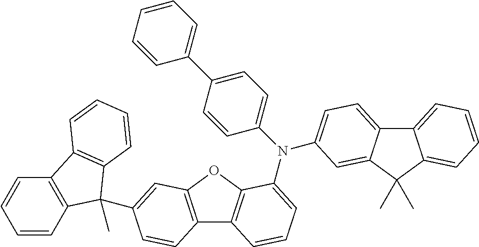 [SMILES: CC1(C)c2ccccc2-c2ccc(N(c3ccc(-c4ccccc4)cc3)c3cccc4c3oc3cc(C5(C)c6ccccc6-c6ccccc65)ccc34)cc21] | 72% |
| 011 | 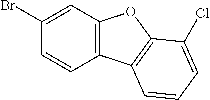 [SMILES: Clc1cccc2c1oc1cc(Br)ccc12] | 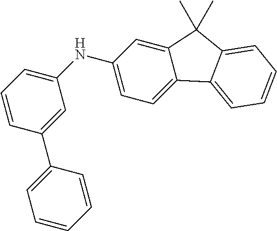 [SMILES: CC1(C)c2ccccc2-c2ccc(Nc3cccc(-c4ccccc4)c3)cc21] | 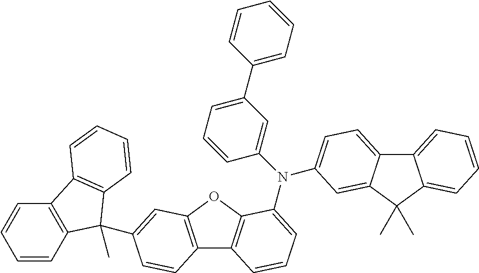 [SMILES: CC1(C)c2ccccc2-c2ccc(N(c3cccc(-c4ccccc4)c3)c3cccc4c3oc3cc(C5(C)c6ccccc6-c6ccccc65)ccc34)cc21] | 79% |
| 014 | 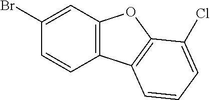 [SMILES: Clc1cccc2c1oc1cc(Br)ccc12] | 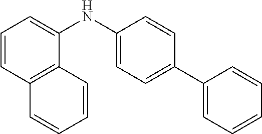 [SMILES: c1ccc(-c2ccc(Nc3cccc4ccccc34)cc2)cc1] | 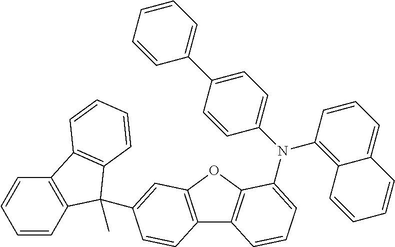 [SMILES: CC1(c2ccc3c(c2)oc2c(N(c4ccc(-c5ccccc5)cc4)c4cccc5ccccc45)cccc23)c2ccccc2-c2ccccc21] | 79% |
| 015 | 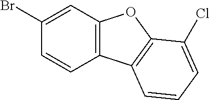 [SMILES: Clc1cccc2c1oc1cc(Br)ccc12] | 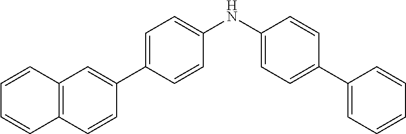 [SMILES: c1ccc(-c2ccc(Nc3ccc(-c4ccc5ccccc5c4)cc3)cc2)cc1] | 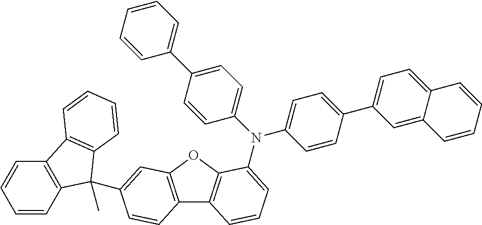 [SMILES: CC1(c2ccc3c(c2)oc2c(N(c4ccc(-c5ccccc5)cc4)c4ccc(-c5ccc6ccccc6c5)cc4)cccc23)c2ccccc2-c2ccccc21] | 68% |
| 023 | 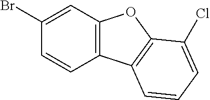 [SMILES: Clc1cccc2c1oc1cc(Br)ccc12] | 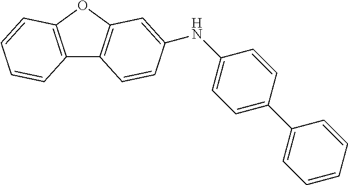 [SMILES: c1ccc(-c2ccc(Nc3ccc4c(c3)oc3ccccc34)cc2)cc1] | 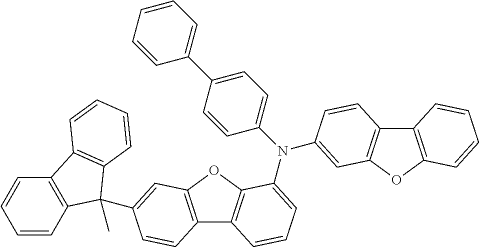 [SMILES: CC1(c2ccc3c(c2)oc2c(N(c4ccc(-c5ccccc5)cc4)c4ccc5c(c4)oc4ccccc45)cccc23)c2ccccc2-c2ccccc21] | 80% |
| 030 | 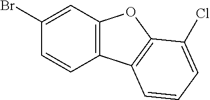 [SMILES: Clc1cccc2c1oc1cc(Br)ccc12] | 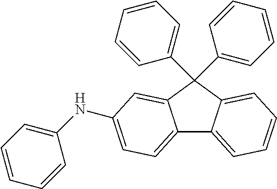 [SMILES: c1ccc(Nc2ccc3c(c2)C(c2ccccc2)(c2ccccc2)c2ccccc2-3)cc1] | 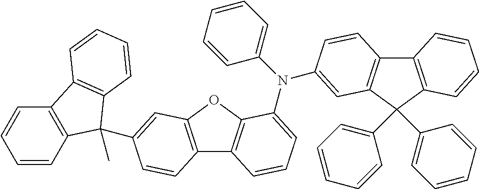 [SMILES: CC1(c2ccc3c(c2)oc2c(N(c4ccccc4)c4ccc5c(c4)C(c4ccccc4)(c4ccccc4)c4ccccc4-5)cccc23)c2ccccc2-c2ccccc21] | 75% |
| 042 | 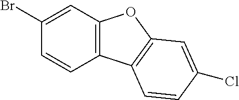 [SMILES: Clc1ccc2c(c1)oc1cc(Br)ccc12] | 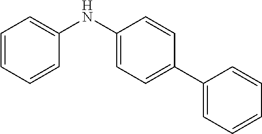 [SMILES: c1ccc(Nc2ccc(-c3ccccc3)cc2)cc1] | 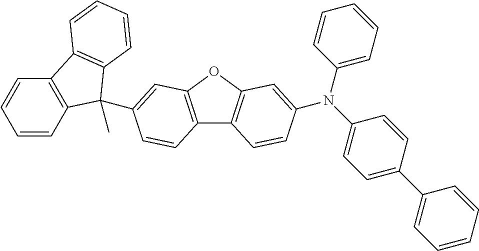 [SMILES: CC1(c2ccc3c(c2)oc2cc(N(c4ccccc4)c4ccc(-c5ccccc5)cc4)ccc23)c2ccccc2-c2ccccc21] | 73% |
| 048 | 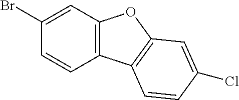 [SMILES: Clc1ccc2c(c1)oc1cc(Br)ccc12] | 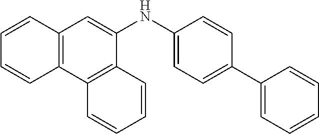 [SMILES: c1ccc(-c2ccc(Nc3cc4ccccc4c4ccccc34)cc2)cc1] | 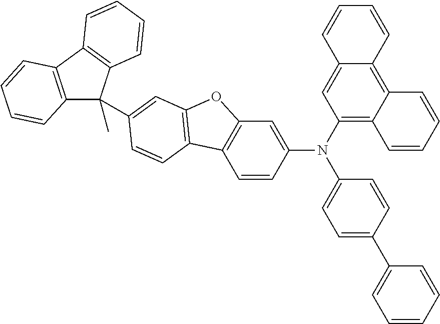 [SMILES: CC1(c2ccc3c(c2)oc2cc(N(c4ccc(-c5ccccc5)cc4)c4cc5ccccc5c5ccccc45)ccc23)c2ccccc2-c2ccccc21] | 79% |
| 058 | 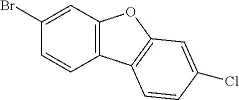 [SMILES: Clc1ccc2c(c1)oc1cc(Br)ccc12] | 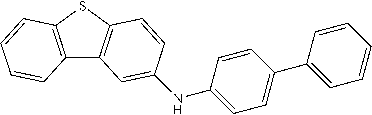 [SMILES: c1ccc(-c2ccc(Nc3ccc4sc5ccccc5c4c3)cc2)cc1] | 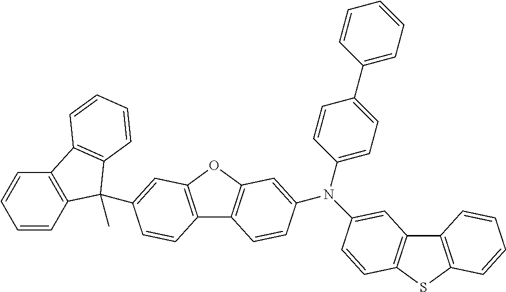 [SMILES: CC1(c2ccc3c(c2)oc2cc(N(c4ccc(-c5ccccc5)cc4)c4ccc5sc6ccccc6c5c4)ccc23)c2ccccc2-c2ccccc21] | 72% |
| 063 | 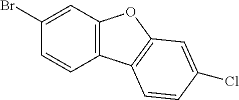 [SMILES: Clc1ccc2c(c1)oc1cc(Br)ccc12] | 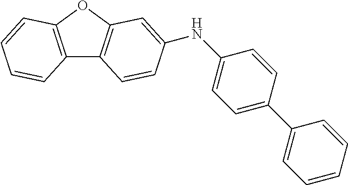 [SMILES: c1ccc(-c2ccc(Nc3ccc4c(c3)oc3ccccc34)cc2)cc1] | 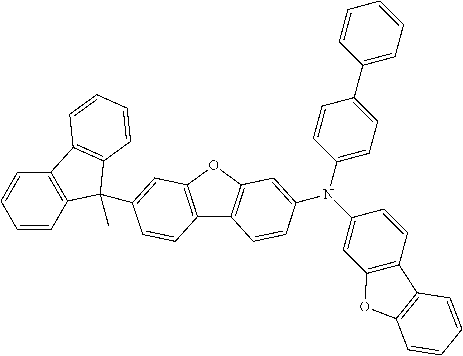 [SMILES: CC1(c2ccc3c(c2)oc2cc(N(c4ccc(-c5ccccc5)cc4)c4ccc5c(c4)oc4ccccc45)ccc23)c2ccccc2-c2ccccc21] | 78% |
| 069 | 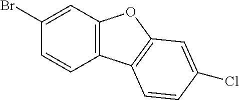 [SMILES: Clc1ccc2c(c1)oc1cc(Br)ccc12] | 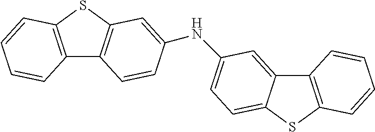 [SMILES: c1ccc2c(c1)sc1cc(Nc3ccc4sc5ccccc5c4c3)ccc12] | 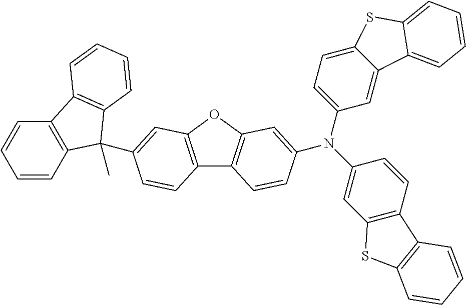 [SMILES: CC1(c2ccc3c(c2)oc2cc(N(c4ccc5c(c4)sc4ccccc45)c4ccc5sc6ccccc6c5c4)ccc23)c2ccccc2-c2ccccc21] | 68% |
| 082 | 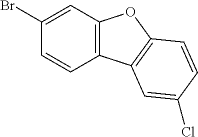 [SMILES: Clc1ccc2oc3cc(Br)ccc3c2c1] | 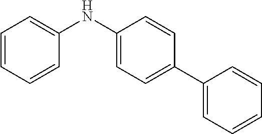 [SMILES: c1ccc(Nc2ccc(-c3ccccc3)cc2)cc1] | 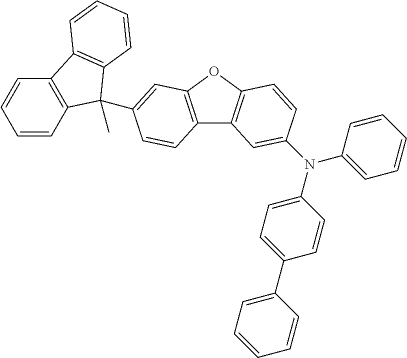 [SMILES: CC1(c2ccc3c(c2)oc2ccc(N(c4ccccc4)c4ccc(-c5ccccc5)cc4)cc23)c2ccccc2-c2ccccc21] | 73% |
| 083 | 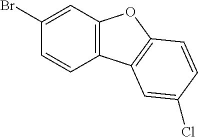 [SMILES: Clc1ccc2oc3cc(Br)ccc3c2c1] | 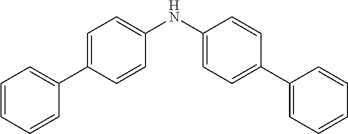 [SMILES: c1ccc(-c2ccc(Nc3ccc(-c4ccccc4)cc3)cc2)cc1] | 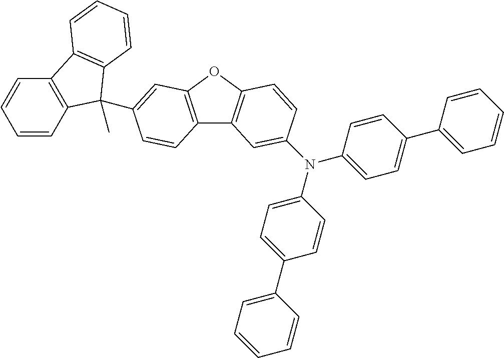 [SMILES: CC1(c2ccc3c(c2)oc2ccc(N(c4ccc(-c5ccccc5)cc4)c4ccc(-c5ccccc5)cc4)cc23)c2ccccc2-c2ccccc21] | 72% |
| 084 | 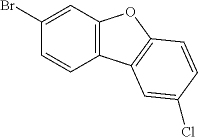 [SMILES: Clc1ccc2oc3cc(Br)ccc3c2c1] | 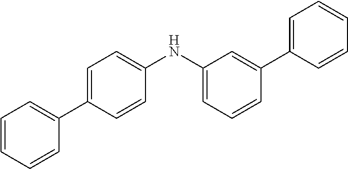 [SMILES: c1ccc(-c2ccc(Nc3cccc(-c4ccccc4)c3)cc2)cc1] | 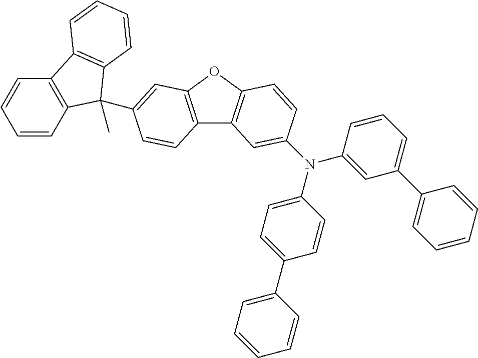 [SMILES: CC1(c2ccc3c(c2)oc2ccc(N(c4ccc(-c5ccccc5)cc4)c4cccc(-c5ccccc5)c4)cc23)c2ccccc2-c2ccccc21] | 75% |
| 097 | 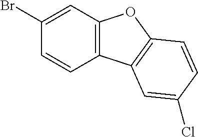 [SMILES: Clc1ccc2oc3cc(Br)ccc3c2c1] | 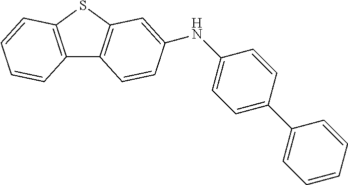 [SMILES: c1ccc(-c2ccc(Nc3ccc4c(c3)sc3ccccc34)cc2)cc1] | 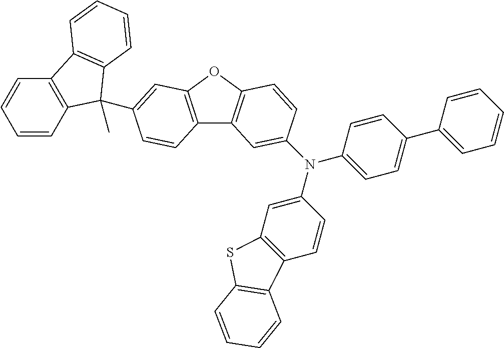 [SMILES: CC1(c2ccc3c(c2)oc2ccc(N(c4ccc(-c5ccccc5)cc4)c4ccc5c(c4)sc4ccccc45)cc23)c2ccccc2-c2ccccc21] | 80% |
| 110 | 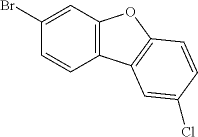 [SMILES: Clc1ccc2oc3cc(Br)ccc3c2c1] | 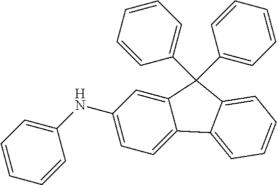 [SMILES: c1ccc(Nc2ccc3c(c2)C(c2ccccc2)(c2ccccc2)c2ccccc2-3)cc1] | 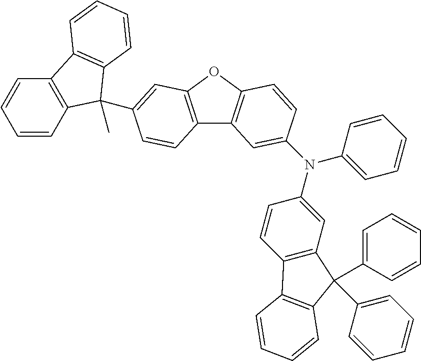 [SMILES: CC1(c2ccc3c(c2)oc2ccc(N(c4ccccc4)c4ccc5c(c4)C(c4ccccc4)(c4ccccc4)c4ccccc4-5)cc23)c2ccccc2-c2ccccc21] | 79% |
| 127 | 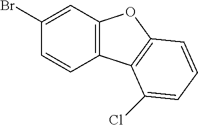 [SMILES: Clc1cccc2oc3cc(Br)ccc3c12] | 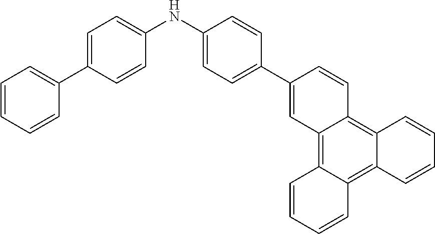 [SMILES: c1ccc(-c2ccc(Nc3ccc(-c4ccc5c6ccccc6c6ccccc6c5c4)cc3)cc2)cc1] | 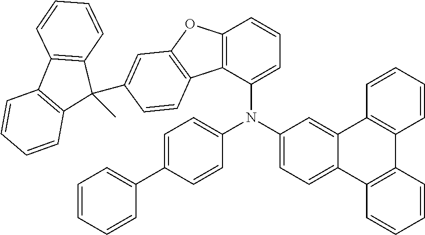 [SMILES: CC1(c2ccc3c(c2)oc2cccc(N(c4ccc(-c5ccccc5)cc4)c4ccc5c6ccccc6c6ccccc6c5c4)c23)c2ccccc2-c2ccccc21] | 74% |
| 135 | 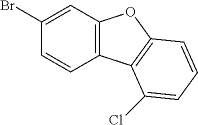 [SMILES: Clc1cccc2oc3cc(Br)ccc3c12] | 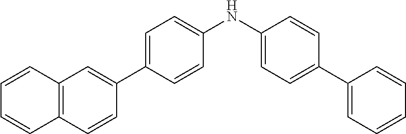 [SMILES: c1ccc(-c2ccc(Nc3ccc(-c4ccc5ccccc5c4)cc3)cc2)cc1] | 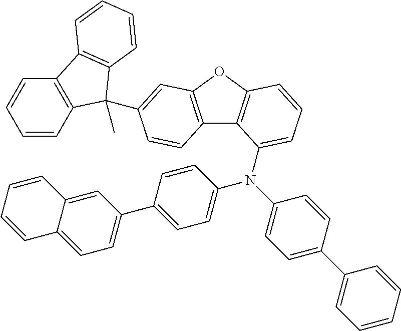 [SMILES: CC1(c2ccc3c(c2)oc2cccc(N(c4ccc(-c5ccccc5)cc4)c4ccc(-c5ccc6ccccc6c5)cc4)c23)c2ccccc2-c2ccccc21] | 71% |
| 138 | 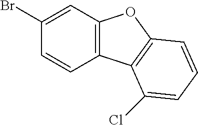 [SMILES: Clc1cccc2oc3cc(Br)ccc3c12] | 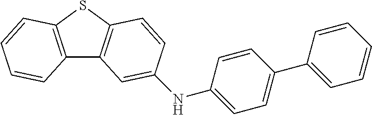 [SMILES: c1ccc(-c2ccc(Nc3ccc4sc5ccccc5c4c3)cc2)cc1] | 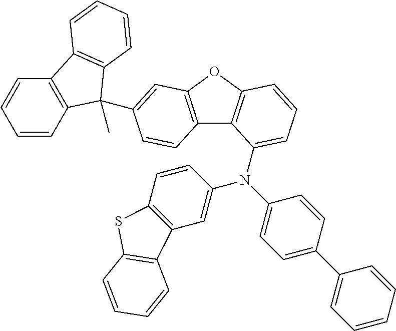 [SMILES: CC1(c2ccc3c(c2)oc2cccc(N(c4ccc(-c5ccccc5)cc4)c4ccc5sc6ccccc6c5c4)c23)c2ccccc2-c2ccccc21] | 76% |
| 175 | 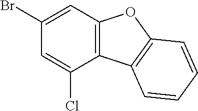 [SMILES: Clc1cc(Br)cc2oc3ccccc3c12] | 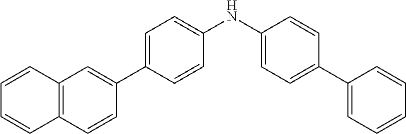 [SMILES: c1ccc(-c2ccc(Nc3ccc(-c4ccc5ccccc5c4)cc3)cc2)cc1] | 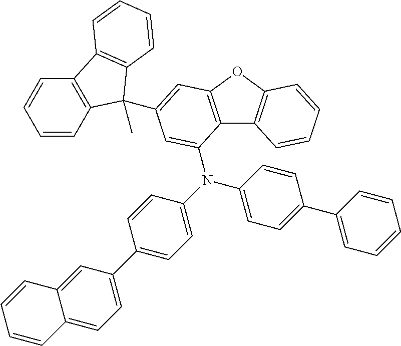 [SMILES: CC1(c2cc(N(c3ccc(-c4ccccc4)cc3)c3ccc(-c4ccc5ccccc5c4)cc3)c3c(c2)oc2ccccc23)c2ccccc2-c2ccccc21] | 73% |
| 203 | 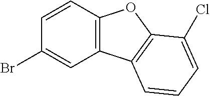 [SMILES: Clc1cccc2c1oc1ccc(Br)cc12] | 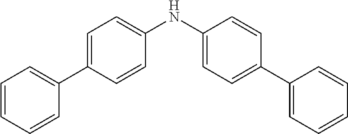 [SMILES: c1ccc(-c2ccc(Nc3ccc(-c4ccccc4)cc3)cc2)cc1] | 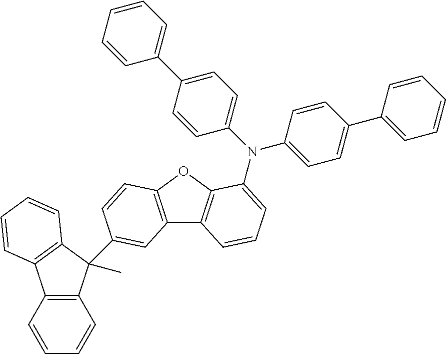 [SMILES: CC1(c2ccc3oc4c(N(c5ccc(-c6ccccc6)cc5)c5ccc(-c6ccccc6)cc5)cccc4c3c2)c2ccccc2-c2ccccc21] | 73% |
| 204 | 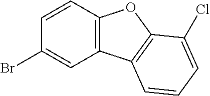 [SMILES: Clc1cccc2c1oc1ccc(Br)cc12] | 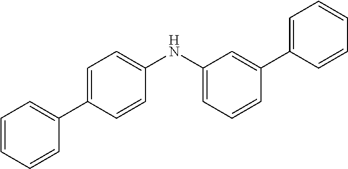 [SMILES: c1ccc(-c2ccc(Nc3cccc(-c4ccccc4)c3)cc2)cc1] | 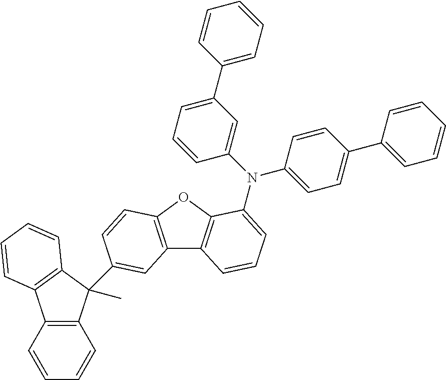 [SMILES: CC1(c2ccc3oc4c(N(c5ccc(-c6ccccc6)cc5)c5cccc(-c6ccccc6)c5)cccc4c3c2)c2ccccc2-c2ccccc21] | 78% |
| 250 | 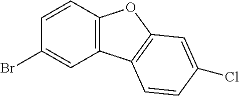 [SMILES: Clc1ccc2c(c1)oc1ccc(Br)cc12] | 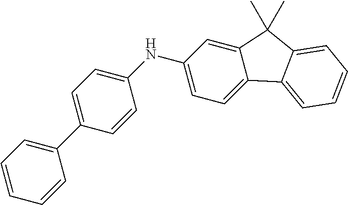 [SMILES: CC1(C)c2ccccc2-c2ccc(Nc3ccc(-c4ccccc4)cc3)cc21] | 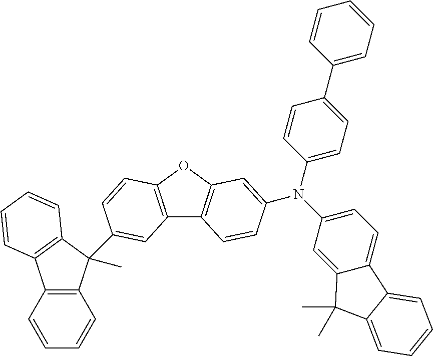 [SMILES: CC1(C)c2ccccc2-c2ccc(N(c3ccc(-c4ccccc4)cc3)c3ccc4c(c3)oc3ccc(C5(C)c6ccccc6-c6ccccc65)cc34)cc21] | 75% |
| 260 | 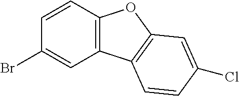 [SMILES: Clc1ccc2c(c1)oc1ccc(Br)cc12] | 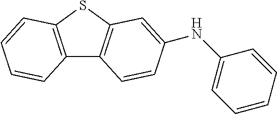 [SMILES: c1ccc(Nc2ccc3c(c2)sc2ccccc23)cc1] | 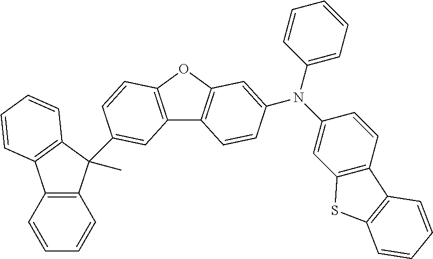 [SMILES: CC1(c2ccc3oc4cc(N(c5ccccc5)c5ccc6c(c5)sc5ccccc56)ccc4c3c2)c2ccccc2-c2ccccc21] | 81% |
| 284 | 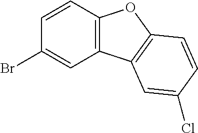 [SMILES: Clc1ccc2oc3ccc(Br)cc3c2c1] | 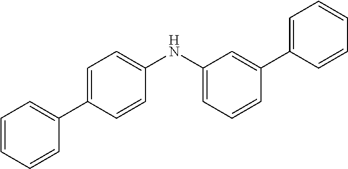 [SMILES: c1ccc(-c2ccc(Nc3cccc(-c4ccccc4)c3)cc2)cc1] | 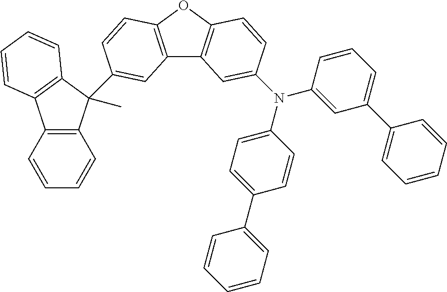 [SMILES: CC1(c2ccc3oc4ccc(N(c5ccc(-c6ccccc6)cc5)c5cccc(-c6ccccc6)c5)cc4c3c2)c2ccccc2-c2ccccc21] | 79% |
| 290 | 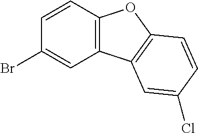 [SMILES: Clc1ccc2oc3ccc(Br)cc3c2c1] | 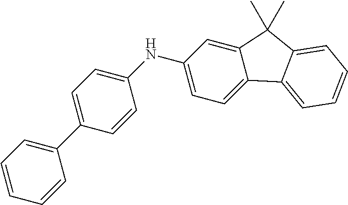 [SMILES: CC1(C)c2ccccc2-c2ccc(Nc3ccc(-c4ccccc4)cc3)cc21] | 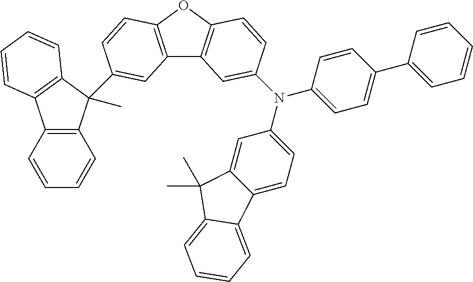 [SMILES: CC1(C)c2ccccc2-c2ccc(N(c3ccc(-c4ccccc4)cc3)c3ccc4oc5ccc(C6(C)c7ccccc7-c7ccccc76)cc5c4c3)cc21] | 77% |
| 293 | 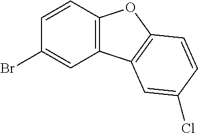 [SMILES: Clc1ccc2oc3ccc(Br)cc3c2c1] | 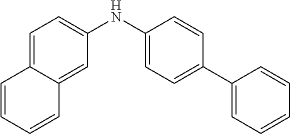 [SMILES: c1ccc(-c2ccc(Nc3ccc4ccccc4c3)cc2)cc1] | 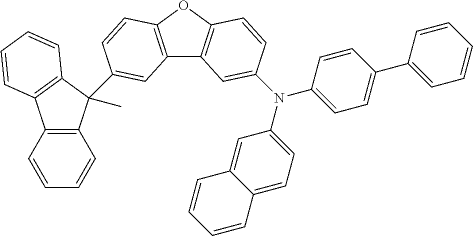 [SMILES: CC1(c2ccc3oc4ccc(N(c5ccc(-c6ccccc6)cc5)c5ccc6ccccc6c5)cc4c3c2)c2ccccc2-c2ccccc21] | 72% |
| 323 | 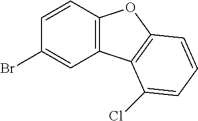 [SMILES: Clc1cccc2oc3ccc(Br)cc3c12] | 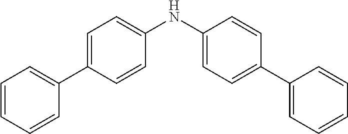 [SMILES: c1ccc(-c2ccc(Nc3ccc(-c4ccccc4)cc3)cc2)cc1] | 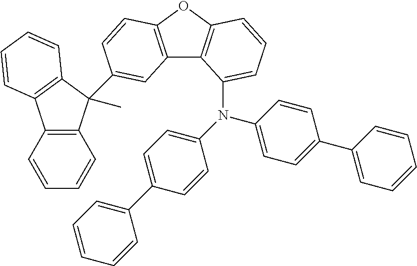 [SMILES: CC1(c2ccc3oc4cccc(N(c5ccc(-c6ccccc6)cc5)c5ccc(-c6ccccc6)cc5)c4c3c2)c2ccccc2-c2ccccc21] | 76% |
| 408 | 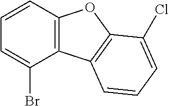 [SMILES: Clc1cccc2c1oc1cccc(Br)c12] | 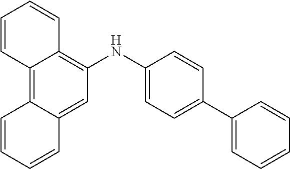 [SMILES: c1ccc(-c2ccc(Nc3cc4ccccc4c4ccccc34)cc2)cc1] | 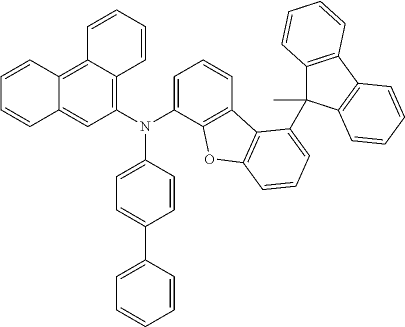 [SMILES: CC1(c2cccc3oc4c(N(c5ccc(-c6ccccc6)cc5)c5cc6ccccc6c6ccccc56)cccc4c23)c2ccccc2-c2ccccc21] | 73% |
| 422 | 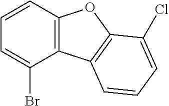 [SMILES: Clc1cccc2c1oc1cccc(Br)c12] | 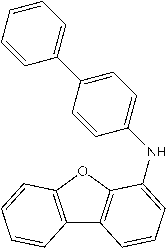 [SMILES: c1ccc(-c2ccc(Nc3cccc4c3oc3ccccc34)cc2)cc1] | 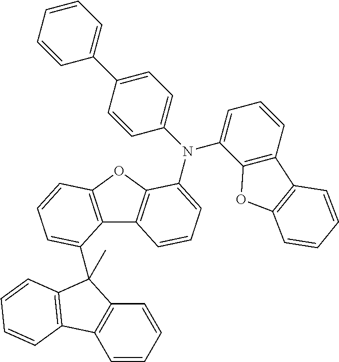 [SMILES: CC1(c2cccc3oc4c(N(c5ccc(-c6ccccc6)cc5)c5cccc6c5oc5ccccc56)cccc4c23)c2ccccc2-c2ccccc21] | 77% |
| 454 | 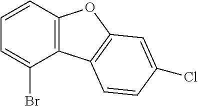 [SMILES: Clc1ccc2c(c1)oc1cccc(Br)c12] | 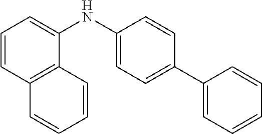 [SMILES: c1ccc(-c2ccc(Nc3cccc4ccccc34)cc2)cc1] | 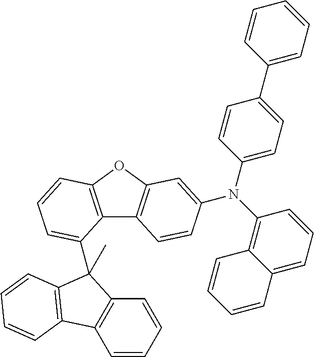 [SMILES: CC1(c2cccc3oc4cc(N(c5ccc(-c6ccccc6)cc5)c5cccc6ccccc56)ccc4c23)c2ccccc2-c2ccccc21] | 82% |
| 464 | 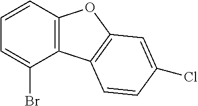 [SMILES: Clc1ccc2c(c1)oc1cccc(Br)c12] | 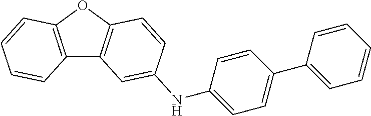 [SMILES: c1ccc(-c2ccc(Nc3ccc4oc5ccccc5c4c3)cc2)cc1] | 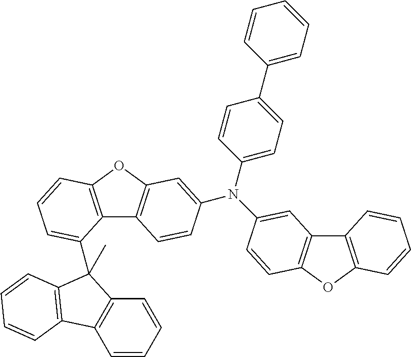 [SMILES: CC1(c2cccc3oc4cc(N(c5ccc(-c6ccccc6)cc5)c5ccc6oc7ccccc7c6c5)ccc4c23)c2ccccc2-c2ccccc21] | 79% |
| 482 | 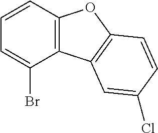 [SMILES: Clc1ccc2oc3cccc(Br)c3c2c1] | 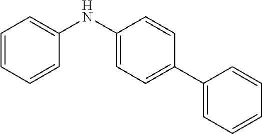 [SMILES: c1ccc(Nc2ccc(-c3ccccc3)cc2)cc1] | 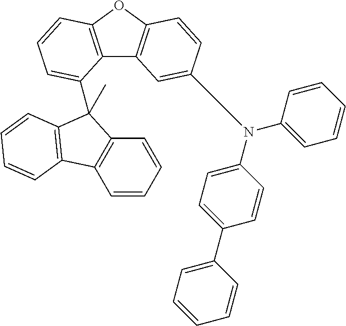 [SMILES: CC1(c2cccc3oc4ccc(N(c5ccccc5)c5ccc(-c6ccccc6)cc5)cc4c23)c2ccccc2-c2ccccc21] | 71% |
| 571 | 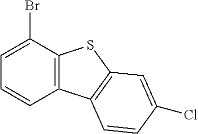 [SMILES: Clc1ccc2c(c1)sc1c(Br)cccc12] | 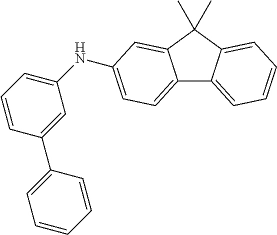 [SMILES: CC1(C)c2ccccc2-c2ccc(Nc3cccc(-c4ccccc4)c3)cc21] | 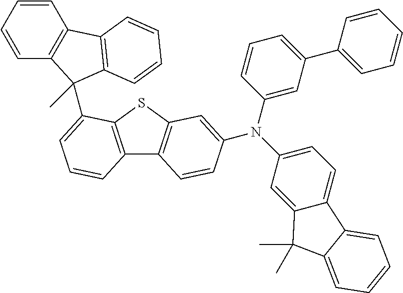 [SMILES: CC1(C)c2ccccc2-c2ccc(N(c3cccc(-c4ccccc4)c3)c3ccc4c(c3)sc3c(C5(C)c6ccccc6-c6ccccc65)cccc34)cc21] | 69% |
| 611 | 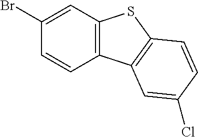 [SMILES: Clc1ccc2sc3cc(Br)ccc3c2c1] | 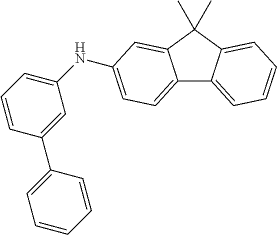 [SMILES: CC1(C)c2ccccc2-c2ccc(Nc3cccc(-c4ccccc4)c3)cc21] | 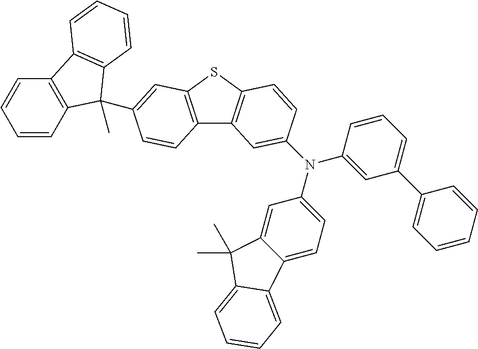 [SMILES: CC1(C)c2ccccc2-c2ccc(N(c3cccc(-c4ccccc4)c3)c3ccc4sc5cc(C6(C)c7ccccc7-c7ccccc76)ccc5c4c3)cc21] | 75% |
| 698 | 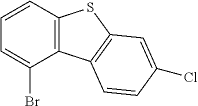 [SMILES: Clc1ccc2c(c1)sc1cccc(Br)c12] | 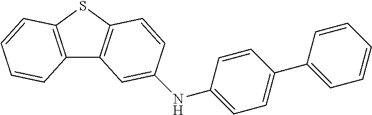 [SMILES: c1ccc(-c2ccc(Nc3ccc4sc5ccccc5c4c3)cc2)cc1] | 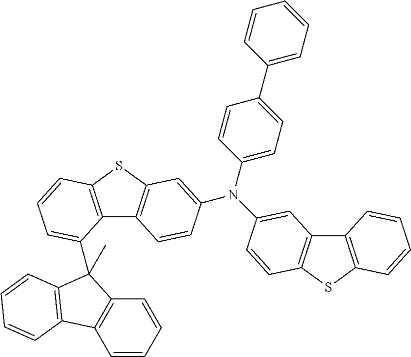 [SMILES: CC1(c2cccc3sc4cc(N(c5ccc(-c6ccccc6)cc5)c5ccc6sc7ccccc7c6c5)ccc4c23)c2ccccc2-c2ccccc21] | 79% |

### <Preparation Example 2> Preparation of Compound 035

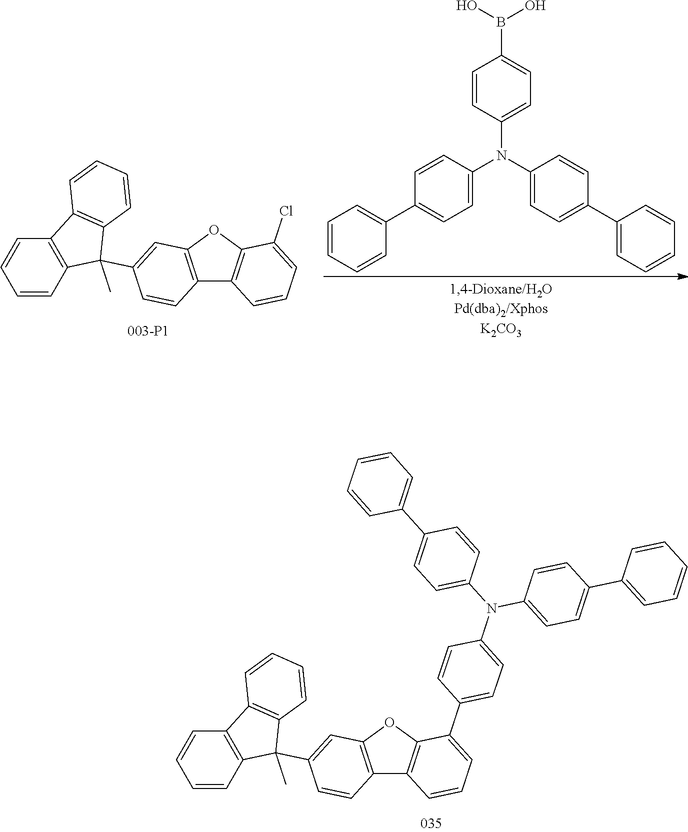

### 1) Preparation of Compound 035

After Compound 003-P1 (10 g, 26.26 mmol) and 4-(di([1,1′-biphenyl]-4-yl)amino)phenyl)boronic acid (11.59 g, 26.26 mmol) were dissolved in 100 ml of 1,4-dioxane and 20 ml of distilled water, Pd(dba)₂ (0.75 g, 1.31 mol), Xphos (1.25 g, 2.63 mmol) and K₂CO₃ (9.07 g, 65.64 mmol) were added thereto, and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 035 (15 g, 77%).

In addition, a target compound in the following Table 2 was synthesized in the same manner as in Preparation Example 2, except that Compound C in the following Table 2 was used instead of Compound 003-P1 and Compound D in the following Table 2 was used instead of 4-(di([1,1′-biphenyl]-4-yl)amino)phenyl)boronic acid, in Preparation Example 2.

TABLE 2

| Compound | | | | |
|---|---|---|---|---|
| No. | Compound C | Compound D | Target compound | yield |
| 117 | 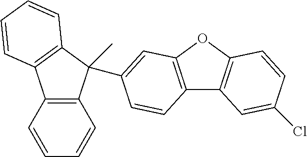 [SMILES: CC1(c2ccc3c(c2)oc2ccc(Cl)cc23)c2ccccc2-c2ccccc21] | 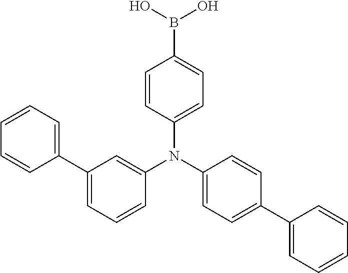 [SMILES: OB(O)c1ccc(N(c2ccc(-c3ccccc3)cc2)c2cccc(-c3ccccc3)c2)cc1] | 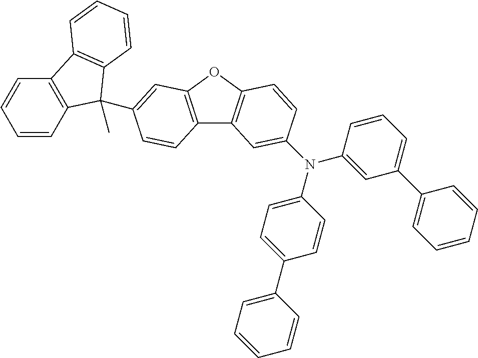 [SMILES: CC1(c2ccc3c(c2)oc2ccc(N(c4ccc(-c5ccccc5)cc4)c4cccc(-c5ccccc5)c4)cc23)c2ccccc2-c2ccccc21] | 81% |
| 155 | 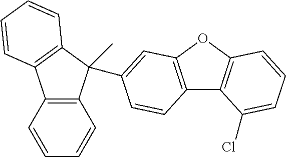 [SMILES: CC1(c2ccc3c(c2)oc2cccc(Cl)c23)c2ccccc2-c2ccccc21] | 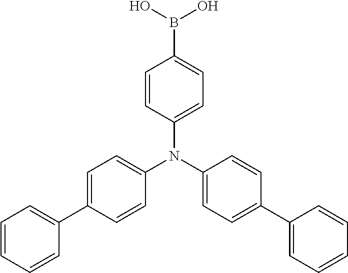 [SMILES: OB(O)c1ccc(N(c2ccc(-c3ccccc3)cc2)c2ccc(-c3ccccc3)cc2)cc1] | 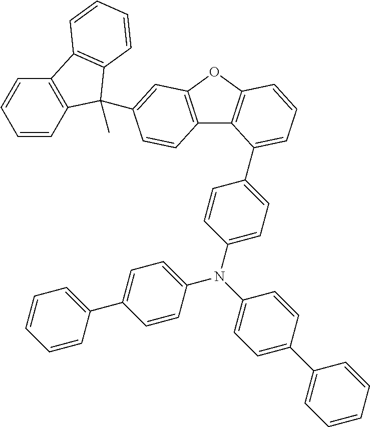 [SMILES: CC1(c2ccc3c(c2)oc2cccc(-c4ccc(N(c5ccc(-c6ccccc6)cc5)c5ccc(-c6ccccc6)cc5)cc4)c23)c2ccccc2-c2ccccc21] | 75% |
| 236 | 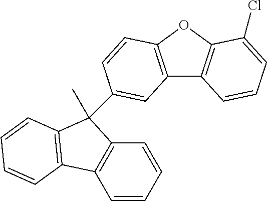 [SMILES: CC1(c2ccc3oc4c(Cl)cccc4c3c2)c2ccccc2-c2ccccc21] | 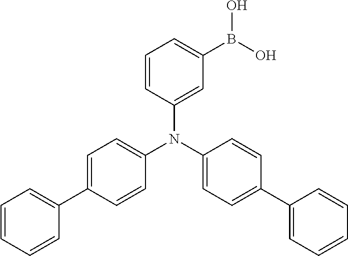 [SMILES: OB(O)c1cccc(N(c2ccc(-c3ccccc3)cc2)c2ccc(-c3ccccc3)cc2)c1] | 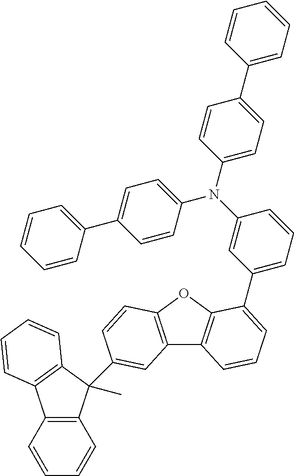 [SMILES: CC1(c2ccc3oc4c(-c5cccc(N(c6ccc(-c7ccccc7)cc6)c6ccc(-c7ccccc7)cc6)c5)cccc4c3c2)c2ccccc2-c2ccccc21] | 79% |
| 273 | 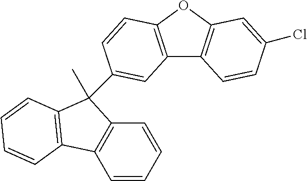 [SMILES: CC1(c2ccc3oc4cc(Cl)ccc4c3c2)c2ccccc2-c2ccccc21] | 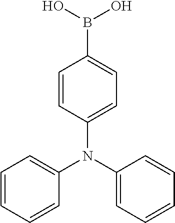 [SMILES: OB(O)c1ccc(N(c2ccccc2)c2ccccc2)cc1] | 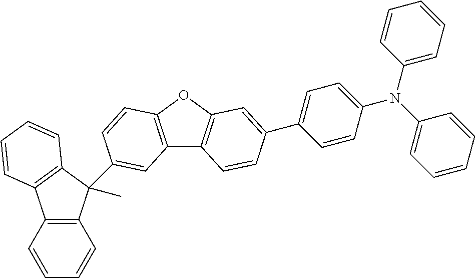 [SMILES: CC1(c2ccc3oc4cc(-c5ccc(N(c6ccccc6)c6ccccc6)cc5)ccc4c3c2)c2ccccc2-c2ccccc21] | 74% |
| 355 | 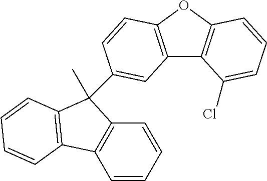 [SMILES: CC1(c2ccc3oc4cccc(Cl)c4c3c2)c2ccccc2-c2ccccc21] | 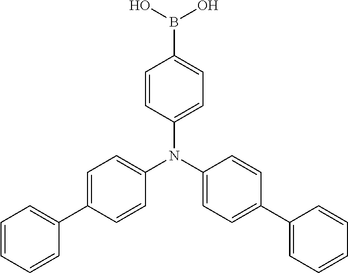 [SMILES: OB(O)c1ccc(N(c2ccc(-c3ccccc3)cc2)c2ccc(-c3ccccc3)cc2)cc1] | 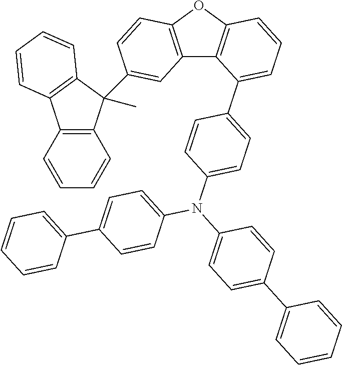 [SMILES: CC1(c2ccc3oc4cccc(-c5ccc(N(c6ccc(-c7ccccc7)cc6)c6ccc(-c7ccccc7)cc6)cc5)c4c3c2)c2ccccc2-c2ccccc21] | 82% |
| 397 | 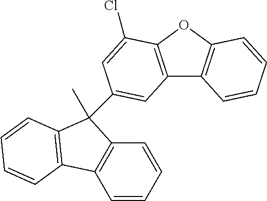 [SMILES: CC1(c2cc(Cl)c3oc4ccccc4c3c2)c2ccccc2-c2ccccc21] | 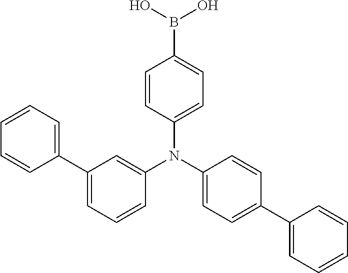 [SMILES: OB(O)c1ccc(N(c2ccc(-c3ccccc3)cc2)c2cccc(-c3ccccc3)c2)cc1] | 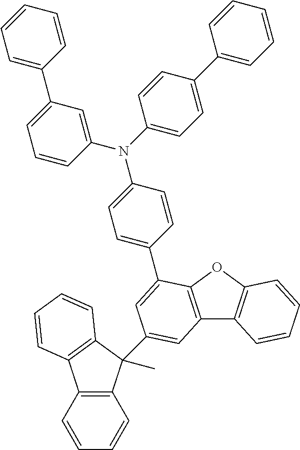 [SMILES: CC1(c2cc(-c3ccc(N(c4ccc(-c5ccccc5)cc4)c4cccc(-c5ccccc5)c4)cc3)c3oc4ccccc4c3c2)c2ccccc2-c2ccccc21] | 78% |
| 635 | 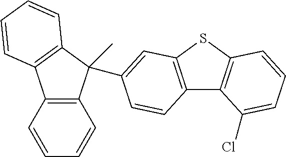 [SMILES: CC1(c2ccc3c(c2)sc2cccc(Cl)c23)c2ccccc2-c2ccccc21] | 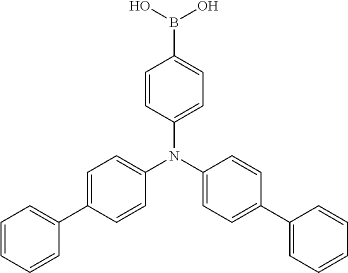 [SMILES: OB(O)c1ccc(N(c2ccc(-c3ccccc3)cc2)c2ccc(-c3ccccc3)cc2)cc1] | 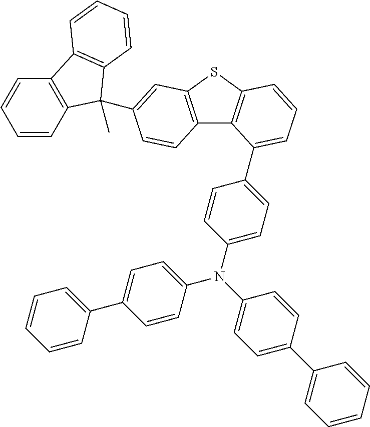 [SMILES: CC1(c2ccc3c(c2)sc2cccc(-c4ccc(N(c5ccc(-c6ccccc6)cc5)c5ccc(-c6ccccc6)cc5)cc4)c23)c2ccccc2-c2ccccc21] | 77% |
| 731 | 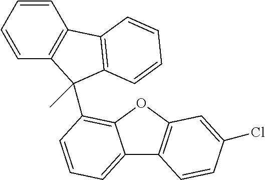 [SMILES: CC1(c2cccc3c2oc2cc(Cl)ccc23)c2ccccc2-c2ccccc21] | 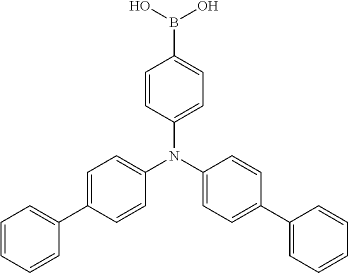 [SMILES: OB(O)c1ccc(N(c2ccc(-c3ccccc3)cc2)c2ccc(-c3ccccc3)cc2)cc1] | 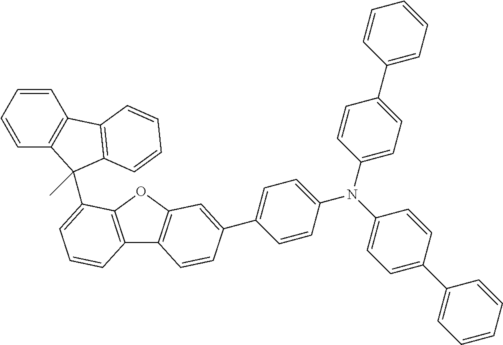 [SMILES: CC1(c2cccc3c2oc2cc(-c4ccc(N(c5ccc(-c6ccccc6)cc5)c5ccc(-c6ccccc6)cc5)cc4)ccc23)c2ccccc2-c2ccccc21] | 81% |
| 743 | 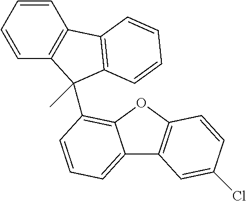 [SMILES: CC1(c2cccc3c2oc2ccc(Cl)cc23)c2ccccc2-c2ccccc21] | 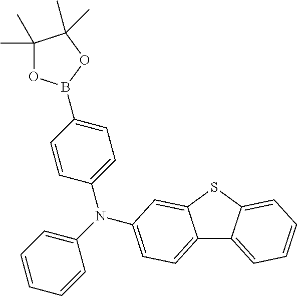 [SMILES: CC1(C)OB(c2ccc(N(c3ccccc3)c3ccc4c(c3)sc3ccccc34)cc2)OC1(C)C] | 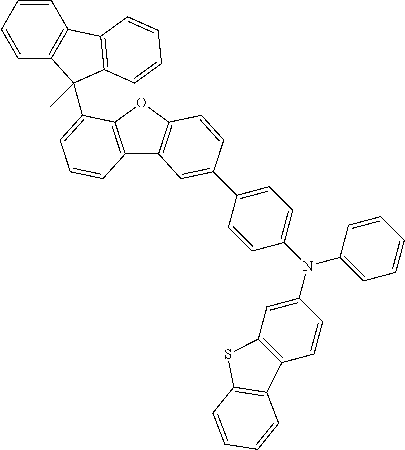 [SMILES: CC1(c2cccc3c2oc2ccc(-c4ccc(N(c5ccccc5)c5ccc6c(c5)sc5ccccc56)cc4)cc23)c2ccccc2-c2ccccc21] | 75% |

### <Preparation Example 3> Preparation of Compound 725

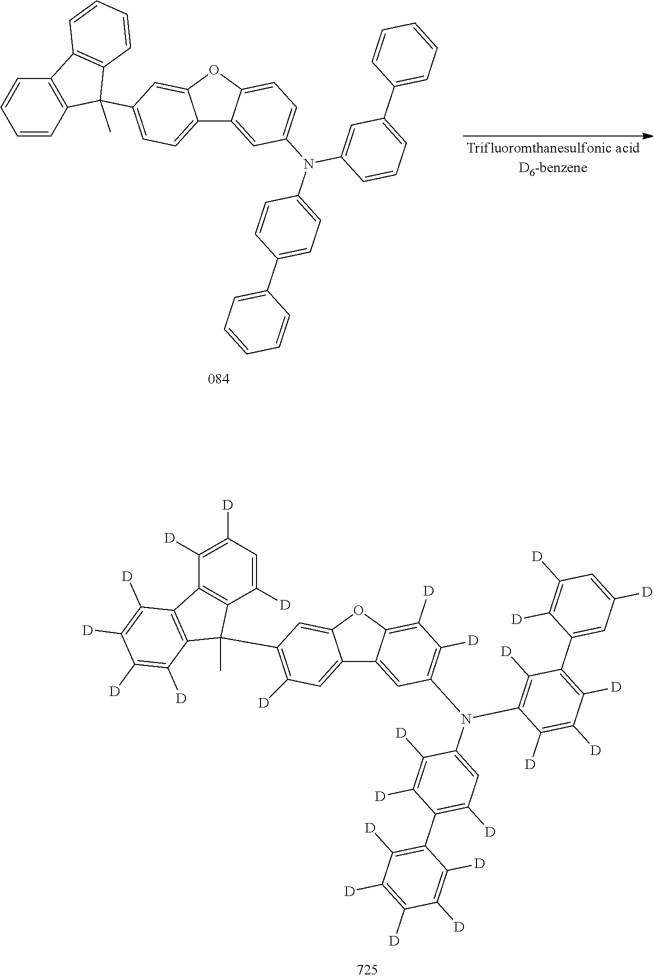 [SMILES: CC1(c2ccc3c(c2)oc2ccc(N(c4ccc(-c5ccccc5)cc4)c4cccc(-c5ccccc5)c4)cc23)c2ccccc2-c2ccccc21 ; [2H]c1cc([2H])c([2H])c(-c2c([2H])c([2H])c([2H])c(N(c3cc([2H])c(-c4c([2H])c([2H])c([2H])c([2H])c4[2H])c([2H])c3[2H])c3cc4c(oc5cc(C6(C)c7c([2H])cc([2H])c([2H])c7-c7c([2H])c([2H])c([2H])c([2H])c76)c([2H])cc54)c([2H])c3[2H])c2[2H])c1]

After Compound 084 (10 g, 15.02 mmol), trifluoromethanesulfonic acid (3.38 g, 22.53 mmol) and 200 ml of D₆-benzene were put into a reaction flask, the resulting mixture was stirred under reflux for 5 hours. After the reaction was completed, the reaction was terminated by adding water thereto, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 725 (8 g, 77%). It was confirmed by LC/MS analysis that 25 deuteriums were substituted on average.

### <Preparation Example 4> Preparation of Compound 741

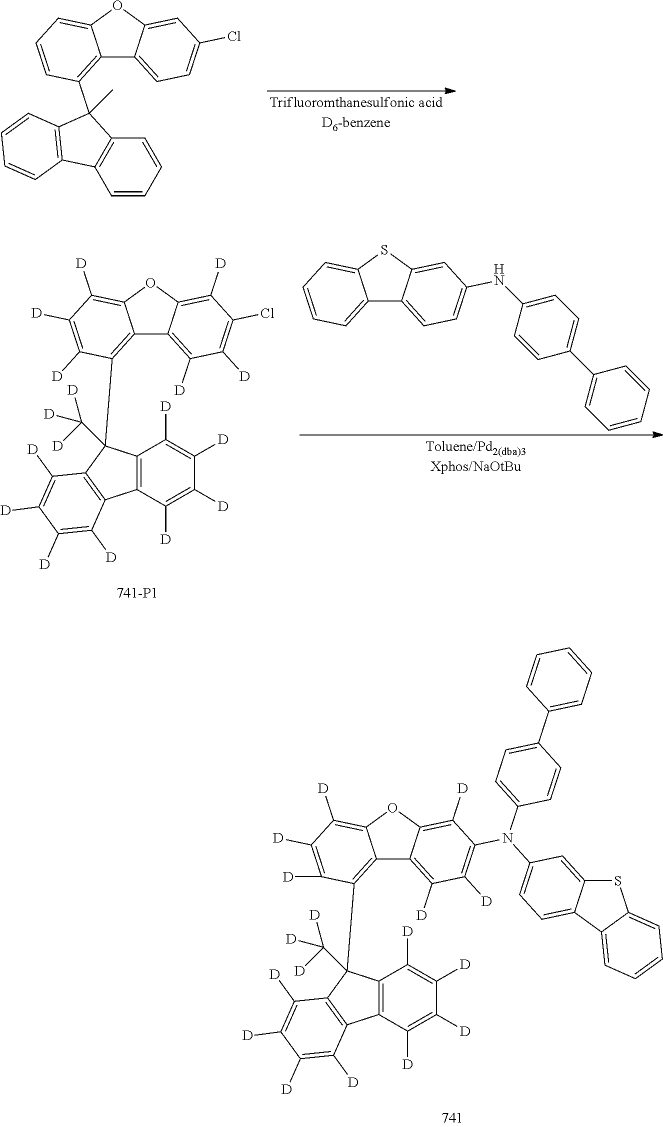

### 1) Preparation of Compound 741-P1

After 7-chloro-1-(9-methyl-9H-fluoren-9-yl)dibenzo[b,d]furan (15 g, 39.38 mmol), trifluoromethanesulfonic acid (8.87 g, 59.08 mmol) and 200 ml of De-benzene were put into a reaction flask, the resulting mixture was stirred under reflux for 5 hours. After the reaction was completed, the reaction was terminated by adding water thereto, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 741-P1 (13 g, 83%).

### 2) Preparation of Compound 741

After Compound 741-P1 (10 g, 25.13 mmol) and N-([1,1′-biphenyl]-4-yl)dibenzo[b,d]thiophen-3-amine (8.83 g, 25.13 mmol) were dissolved in 100 ml of toluene, Pd₂(dba)₃ (1.15 g, 1.26 mmol), Xphos (1.20 g, 2.51 mmol), and sodium tert-butoxide (BuONa) (4.83 g, 50.26 mmol) were added thereto, and the resulting mixture was stirred under reflux for 2 hours. After the reaction was completed, dichloromethane was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 741 (13 g, 73%).

### <Preparation Example 5> Preparation of Compound 742

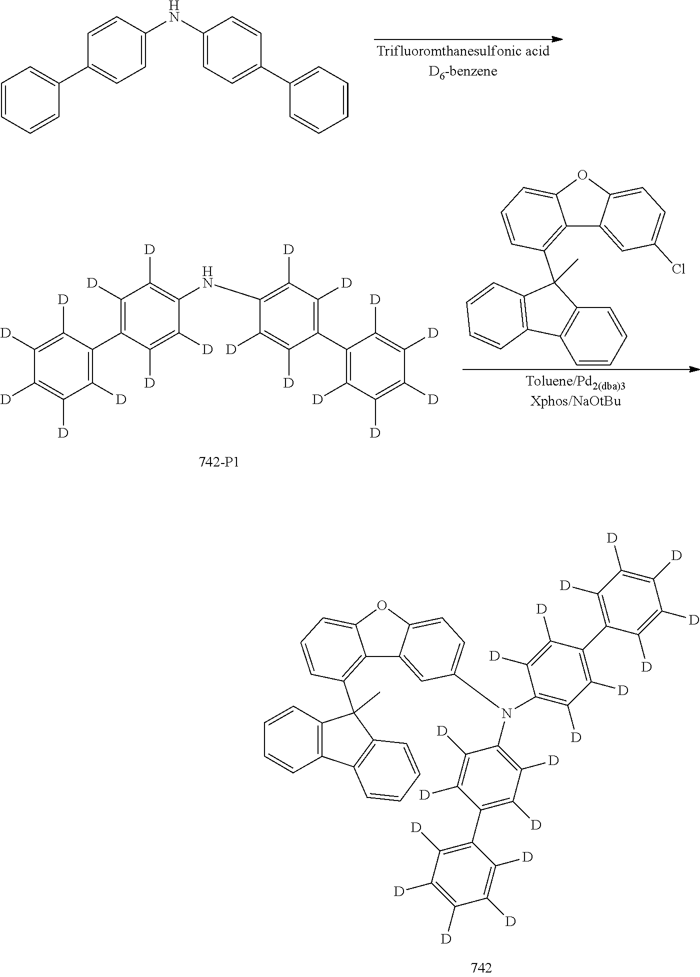

### 1) Preparation of Compound 742-P1

After di([1,1′-biphenyl]-4-yl)amine (15 g, 46.67 mmol), trifluoromethanesulfonic acid (10.51 g, 70 mmol) and 200 ml of De-benzene were put into a reaction flask, the resulting mixture was stirred under reflux for 5 hours. After the reaction was completed, the reaction was terminated by adding water thereto, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 742-P1 (12 g, 76%).

### 2) Preparation of Compound 742

After Compound 742-P1 (10 g, 29.45 mmol) and 8-chloro-1-(9-methyl-9H-fluoren-9-yl)dibenzo[b,d]furan (11.22 g, 29.45 mmol) were dissolved in 100 ml of toluene, Pd₂(dba)₃ (1.35 g, 1.47 mmol), Xphos (1.40 g, 2.95 mmol), and sodium tert-butoxide (BuONa) (5.66 g, 58.90 mmol) were added thereto, and the resulting mixture was stirred under reflux for 2 hours. After the reaction was completed, dichloromethane was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 742 (15 g, 74%).

The preparation examples and the synthesis confirmation results are shown in Tables 3 and 4. Table 3 is about the measurement values of 1H NMR (CDCl₃, 300 MHZ), and Table 4 is about the measurement values of field desorption mass spectrometry (FD-MS).

TABLE 3

| Compound | ¹H NMR(CDCl₃, 300 MHz) |
|---|---|
| 003 | δ = 7.87-7.84 (3H, m), 7.55-7.38 (18H, m), 7.28- |
| | 7.23 (4H, m), 7.07 (1H, t), 6.99 (1H, d), 6.69 |
| | (4H, d), 6.39 (1H, d), 2.28 (3H, s) |
| 010 | δ = 7.87-7.84 (4H, m), 6.72 (1H, d), 7.55-7.51 |
| | (9H, m), 7.41-7.38 (4H, m), 7.28-7.23 (5H, m), |
| | 7.07 (1H, t), 6.99 (1H, d), 6.75 (1H, s), 6.69 |
| | (2H, d), 6.58 (1H, d), 6.39 (1H, d), 2.28 (3H, |
| | s), 1.72 (6H, s) |
| 011 | δ = 7.87-7.84 (4H, m), 7.62 (1H, d), 7.55-7.38 |
| | (13H, m), 7.28-7.23 (5H, m), 7.07 (1H, t), 6.99 |
| | (1H, d), 6.89-6.88 (2H, m), 6.75 (1H, s), 6.59- |
| | 6.58 (2H, m), 6.39 (1H, d), 2.28 (3H, s), 1.72 |
| | (6H, s) |
| 014 | δ = 8.07 (1H, d), 8.02 (1H, d), 7.87(2H, d), 7.84 |
| | (1H, d), 7.57-7.38 (15H, m), 7.28-7.23 (4H, m), |
| | 7.07 (1H, t), 6.99-6.98 (2H, m), 6.69 (2H, d), |
| | 6.38 (1H, d) 2.28 (3H, s) |
| 015 | δ = 8.00 (2H, d), 7.92-7.84 (4H, m), 7.73 (1H, d), |
| | 7.59-7.41 (13H, m), 7.41-7.39 (3H, m), 7.28-7.23 |
| | (4H, m), 7.07 (1H, t), 6.99 (1H, d), 6.69 (4H, |
| | d), 6.39 (1H, d). |
| 023 | δ = 7.89-7.84 (4H, m), 7.66-7.64 (2H, m), 7.55- |
| | 7.28 (18H, m), 7.07 (1H, t), 6.99 (1H, d), 6.69 |
| | (2H, d), 6.39-6.33 (2H, m), 2.28 (3H, s) |
| 030 | δ = 7.87-7.84 (4H, m), 7.62 (1H, d), 7.55 (3H, d), |
| | 7.38-6.99 (22H, m), 6.81 (1H, t), 6.75 (1H, s), |
| | 6.63-6.58 (3H, m), 6.39 (1H, d), 2.28 (3H, s) |
| 035 | δ = 7.87-7.81 (5H, m), 7.55-7.38 (21H, m), 7.28 |
| | (2H, t) 7.23 (1H, s), 6.99 (1H, d), 6.69 (6H, d), |
| | 2.28 (3H, s) |
| 042 | δ = 7.87-7.84 (3H, m), 7.64 (1H, d), 7.55-7.38 |
| | (12H, m), 7.28-7.20 (5H, m), 6.99 (1H, d), 6.81 |
| | (1H, t), 6.69 (2H, d), 6.63 (2H, d), 6.33 (1H, |
| | d), 2.28 (3H, s) |
| 048 | δ = 8.93 (2H, d), 8.12 (2H, d), 7.88-7.82 (7H, m), |
| | 7.64 (1H, d), 7.55-7.38 (12H, m), 7.28 (2H, t), |
| | 7.23 (1H, s), 6.99 (1H, d), 6.91 (1H, s), 6.69 |
| | (2H, d), 6.33 (1H, d), 2.28 (3H, s) |
| 058 | δ = 8.45 (1H, d), 7.98 (1H, d), 7.87-7.84 (3H, m), |
| | 7.73 (1H, d), 7.64 (1H, d), 7.55-7.38 (15H, m), |
| | 7.28 (2H, t), 7.23 (1H, s) 6.99 (1H, d), 6.86 |
| | (1H, d), 6.69 (H, d), 6.33 (1H, d), 2.28 (3H, s) |
| 063 | δ = 7.89-7.84 (4H, m), 7.66-7.64 (3H, m), 7.55- |
| | 7.28 (18H, m), 6.99 (1H, d), 6.69 (2H, d), 6.33 |
| | (2H, d), 2.28 (3H, s) |
| 069 | δ = 8.45 (2H, d), 7.98 (2H, d), 7.87-7.73 (5H, m), |
| | 7.64 (1H, d), 7.55-7.23 (13H, m), 7.06 (1H, s), |
| | 6.99 (1H, d), 6.88-6.86 (2H, m), 6.33 (1H, d), |
| | 2.28 (3H, s) |
| 082 | δ = 7.87-7.84 (3H, m), 7.65 (1H, s), 7.55-7.38 |
| | (12H, m), 7.28-7.20 (5H, m), 6.99 (1H, d), 6.81 |
| | (1H, t), 6.69 (2H, d), 6.63 (2H, d), 6.39 (1H, |
| | d), 2.28 (3H, s) |
| 083 | δ = 7.87-7.84 (3H, m), 7.65 (1H, s), 7.55-7.38 |
| | (19H, m), 7.28 (2H, t), 7.23 (1H, d), 6.99 (1H, |
| | d), 6.69 (4H, d), 6.39 (1H, d), 2.28 (3H, s) |
| 084 | δ = 7.87-7.84 (3H, m), 7.65 (1H, s), 7.55-7.38 |
| | (18H, m), 7.28 (2H, t), 7.23 (1H, s), 6.99 (1H, |
| | d), 6.89-6.88 (2H, m), 6.69 (2H, d), 6.59 (1H, |
| | d), 6.39 (1H, d), 2.28 (3H, s) |
| 097 | δ = 8.45 (1H, d), 7.98 (1H, d), 7.87-7.80 (4H, m), |
| | 7.65 (H, s), 7.55-7.38 (14H, m), 7.28-7.23 (3H, |
| | m), 7.06 (1H, s), 6.88 (1H, d), 6.69 (2H, d), |
| | 6.39 (1H, d), 2.28 (3H, s) |
| 110 | δ = 7.87-7.84 (4H, m), 7.65-7.55 (5H, m), 7.41- |
| | 7.20 (20H, m), 6.99 (1H, d), 6.85-6.81 (2H, m), |
| | 6.63-6.58 (3H, m), 6.39 (1H, d), 2.28 (3H, s) |
| 117 | δ = 7.87-7.81 (4H, m), 7.72-7.71 (2H, m), 7.55- |
| | 7.38 (19H, m), 7.28 (2H, t), 7.23 (1H, s), 6.99 |
| | (1H, d), 6.89-6.88 (2H, m), 6.69 (4H, d), 6.59 |
| | (1H, d), 2.28 (3H, s) |
| 127 | δ = 8.93 (2H, d), 8.13-8.12 (3H, m), 7.88-7.82 |
| | (8H, m), 7.55-7.38 (11H, m), 7.28-7.23 (3H, m), |
| | 7.13 (1H, t), 7.02-6.99 (3H, m), 6.69 (2H, d), |
| | 6.33 (1H, d), 2.28 (3H, s) |
| 135 | δ = 8.00 (2H, d), 7.92-7.84 (4H, m), 7.73 (1H, d), |
| | 7.59-7.51 (13H, m), 7.41 (1H, t), 7.38 (2H, t), |
| | 7.28 (2H, t), 7.23 (1H, s), 7.13 (1H, t), 702- |
| | 6.99 (2H, m), 6.69 (4H, d), 6.33 (1H, d), 2.28 |
| | (3H, s) |
| 138 | δ = 8.45 (1H, d), 7.98 (1H, d), 7.87-7.84 (3H, m), |
| | 7.73 (1H, d), 7.55-7.38 (14H, m), 7.28-7.23 (3H, |
| | m), 7.13 (1H, t), 7.02-6.99 (2H, m), 6.86 (1H, |
| | d), 6.69 (2H, d), 6.33 (1H, d), 2.28 (3H, s) |
| 155 | δ = 7.87 (2H, d), 7.84 (1H, d), 7.75 (1H, d), |
| | 7.62-7.38 (22H, m), 7.28 (2H, t), 7.23 (1H, s), |
| | 6.99 (1H, d), 6.69 (6H, d), 2.28 (3H, s) |
| 175 | δ = 8.00 (2H, d), 7.92-7.87 (4H, m), 7.73-7.28 |
| | (22H, m), 6.69 (4H, d), 6.19 (1H, s), 2.28 (3H, s) |
| 203 | δ = 7.87 (2H, d), 7.61-7.35 (20H, m), 7.28 (2H, |
| | t), 7.25 (1H, d), 7.07-7.05 (2H, m), 6.69 (4H, |
| | d), 6.39 (1H, d), 2.28 (3H, s) |
| 204 | δ = 7.87 (2H, d), 7.61-7.35 (19H, m), 7.28 (2H, |
| | t), 7.25 (1H, d), 7.07-7.05 (2H, m), 6.89-6.88 |
| | (2H, m), 6.69 (2H, d), 6.59 (1H, d), 6.39 (1H, |
| | d), 2.28 (3H, s) |
| 236 | δ = 7.87-7.81 (4H, m), 7.61-7.38 (22H, m), 7.28 |
| | (2H, t), 7.05 (1H, d), 6.89-6.88 (2H, m), 6.69 |
| | (4H, d), 6.59 (1H, d), 2.28 (3H, s) |
| 250 | δ = 7.87 (3H, d), 7.64-7.28 (21H, m), 7.05 (1H, |
| | d), 6.75 (1H, s), 6.69 (2H, d), 6.58 (1H, d), |
| | 6.33 (1H, d), 2.28 (3H, s), 1.72 (6H, s) |
| 260 | δ = 8.45 (1H, d), 7.98 (1H d), 7.87 (2H, d), 7.80 |
| | (1H, d), 7.64-7.20 (14H, m), 7.06-7.05 (2H, m), |
| | 6.88-6.81 (2H, m), 6.63 (1H, d), 6.33 (1H, d), |
| | 2.28 (3H, s) |
| 273 | δ = 7.95 (1H, d), 7.87 (2H, d), 7.75 (1H, d), |
| | 7.64-7.54 (6H, m), 7.38-7.20 (9H, m), 7.05 (1H, |
| | d), 6.81 (2H, t), 6.63 (6H, d), 2.28 (3H, s) |
| 284 | δ = 7.87 (2H, d), 7.65-7.35 (21H, m), 7.28 (2H, |
| | t), 7.05 (1H, d), 6.89-6.88 (2H, m), 6.69 (2H, |
| | d), 6.59 (1H, d), 6.39 (1H, d), 2.28 (3H, s) |
| 290 | δ = 7.87 (3H, d), 7.65-7.28 (21H, m), 7.05 (1H, |
| | d), 6.75 (1H, s) 6.69 (2H, d), 6.58 (1H, d), 6.39 |
| | (1H, d), 2.28 (3H, s), 1.72 (6H, s) |
| 293 | δ = 7.88-7.74 (6H, m), 7.65-7.28 (20H, m), 7.05 |
| | (1H, d), 6.69 (2H, d), 6.39 (1H, d), 2.28 (3H, s) |
| 323 | δ = 7.87 (2H, d), 7.61-7.35 (20H, m), 7.28 (2H, |
| | t), 7.13 (1H, t), 7.05-7.02 (2H, m), 6.69 (4H, |
| | d), 6.33 (1H, d), 2.28 (3H, s) |
| 355 | δ = 7.87 (2H, d), 7.75 (1H, d), 7.62-7.38 (24H, |
| | m), 7.28 (2H, t), 7.05 (1H, d), 6.69 (6H, d), |
| | 2.28 (3H, s) |
| 397 | δ = 7.89-7.87 (3H, m), 7.66 (1H, d), 7.55-7.28 |
| | (25H, m), 6.89-6.88 (2H, m), 6.69 (4H, d), 6.59 |
| | (1H, d), 2.28 (3H, s) |
| 408 | δ = 8.93 (2H, d), 8.12 (2H, d), 7.88-7.82 (6H, m), |
| | 7.55-7.28 (16H, m), 7.07 (1H, t), 6.99 (1H, d), |
| | 6.91 (1H, s), 6.69 (2H, d), 6.39 (1H, d), 2.38 |
| | (3H, s) |
| 422 | δ = 7.89-7.87 (3H, m), 7.66 (1H, d), 7.55-7.25 |
| | (19H, m), 7.07 (2H, t), 6.99 (1H, d), 6.69 (2H, |
| | d), 6.39 (2H, d), 2.28 (3H, s) |
| 454 | δ = 8.07 (1H, d), 8.02 (1H, d), 7.87 (2H, d), |
| | 7.64-7.28 (21H, m), 6.99-6.98 (2H, m), 6.69 (2H, |
| | d), 6.33 (1H, d), 2.28 (3H, s) |
| 464 | δ = 7.89-7.87 (3H, m), 7.66-7.64 (3H, m), 7.55- |
| | 7.28 (19H, m), 6.99 (1H, d), 6.69 (2H, d), 6.39 |
| | (1H, d), 6.33 (1H, d), 2.28 (3H, s) |
| 482 | δ = 7.87 (2H, d), 7.65 (1H, s), 7.55-7.20 (18H, |
| | m), 6.99 (1H, d), 6.81 (1H, t), 6.69 (2H, d), |
| | 6.63 (2H, d), 6.39 (1H, d), 2.28 (3H, s) |
| 571 | δ = 8.27 (1H, d), 7.87 (3H, d), 7.80 (1H, d), |
| | 7.62-7.28 (18H, m), 7.08 (1H, s), 6.89-6.88 (3H, |
| | m), 6.75 (1H, s), 6.59-6.58 (2H, m), 2.28 (3H, |
| | s), 1.72 (6H, s) |
| 611 | δ = 8.00 (1H, d), 7.87 (2H, d), 7.73 (1H, d), 7.72 |
| | (1H, s), 7.55-7.28 (18H, m), 6.89-6.86 (3H, m), |
| | 6.75 (1H, s), 6.59-6.58 (2H, m), 2.28 (3H, s), |
| | 1.72 (6H, s) |
| 635 | δ = 8.00 (1H, d), 7.94 (1H, d), 7.87 (2H, d), 7.82 |
| | (1H, d), 7.72 (1H, s), 7.56-7.28 (24H, m), 6.69 |
| | (6H, d), 2.28 (3H, s) |
| 698 | δ = 8.45 (1H, d), 7.98 (1H, d), 7.87 (2H, d), 7.80 |
| | (2H, d), 7.73 (1H, d), 7.55-7.28 (18H, m), 7.06 |
| | (1H, s), 6.88-6.86 (2H, m), 6.69 (2H, d), 2.28 |
| | (3H, s) |
| 725 | δ = 7.75 (1H, s), 7.66 (1H, s), 7.65 ((1H, s), |
| | 7.40 (1H, s), 7.23 (1H, s), 7.07 (1H, s), 6.70 |
| | (1H, s), 2.28 (3H, s) |
| 741 | δ = 8.45 1(H, d), 7.98 (1H, d), 7.80 (1H, d), |
| | 7.54-7.41 (9H, m), 7.06 (1H, s), 6.88 (1H, d), |
| | 6.69 (2H, d) |
| 742 | δ = 7.87 (2H, d), 7.65 (1H, s), 7.55-7.28 (9H, m), |
| | 6.99 (1H, d), 6.39 (1H, d), 2.28 (3H, s) |

TABLE 4

| Com- | | Com- | |
|---|---|---|---|
| pound | FD-MS | pound | FD-MS |
| 003 | m/z = 665.82 | 010 | m/z = 705.88 |
| | (C₅₀H₃₅NO = 665.27) | | (C₅₃H₃₉NO = 705.30) |
| 011 | m/z = 705.88 | 014 | m/z = 639.78 |
| | (C₅₃H₃₉NO = 705.30) | | (C₄₈H₃₃NO = 639.26) |
| 015 | m/z = 715.88 | 023 | m/z = 679.80 |
| | (C₅₄H₃₇NO = 715.29) | | (C₅₀H₃₃NO₂ = 679.25) |
| 030 | m/z = 753.93 | 035 | m/z = 741.91 |
| | (C₅₇H₃₉NO = 753.30) | | (C₅₆H₃₉NO = 741.30) |
| 042 | m/z = 589.72 | 048 | m/z = 689.84 |
| | (C₄₄H₃₁NO = 589.24) | | (C₅₂H₃₅NO = 689.27) |
| 058 | m/z = 695.87 | 063 | m/z = 679.80 |
| | (C₅₀H₃₃NOS = 695.23) | | (C₅₀H₃₃NO₂ = 679.25) |
| 069 | m/z = 725.92 | 082 | m/z = 589.72 |
| | (C₅₀H₃₁NOS₂ = 725.18) | | (C₄₄H₃₁NO = 589.24) |
| 083 | m/z = 665.82 | 084 | m/z = 665.82 |
| | (C₅₀H₃₅NO = 665.27) | | (C₅₀H₃₅NO = 665.27) |
| 097 | m/z = 695.87 | 110 | m/z = 753.93 |
| | (C₅₀H₃₃NOS = 695.23) | | (C₅₇H₃₉NO = 753.30) |
| 117 | m/z = 741.91 | 127 | m/z = 739.90 |
| | (C₅₆H₃₉NO = 741.30) | | (C₅₆H₃₇NO = 739.29) |
| 135 | m/z = 715.88 | 138 | m/z = 695.87 |
| | (C₅₄H₃₇NO = 715.29) | | (C₅₀H₃₃NOS = 695.23) |
| 155 | m/z = 741.91 | 175 | m/z = 715.88 |
| | (C₅₆H₃₉NO = 741.30) | | (C₅₄H₃₇NO = 715.29) |
| 203 | m/z = 665.82 | 204 | m/z = 665.82 |
| | (C₅₀H₃₅NO = 665.27) | | (C₅₀H₃₅NO = 665.27) |
| 236 | m/z = 741.91 | 250 | m/z = 705.88 |
| | (C₅₆H₃₉NO = 741.30) | | (C₅₃H₃₉NO = 705.30) |
| 260 | m/z = 619.77 | 273 | m/z = 589.72 |
| | (C₄₄H₂₉NOS = 619.20) | | (C₄₄H₃₁NO = 589.24) |
| 284 | m/z = 665.82 | 290 | m/z = 705.88 |
| | (C₅₀H₃₅NO = 665.27) | | (C₅₃H₃₉NO = 705.30) |
| 293 | m/z = 639.78 | 323 | m/z = 665.82 |
| | (C₄₈H₃₃NO = 639.26) | | (C₅₀H₃₅NO = 665.27) |
| 355 | m/z = 741.91 | 397 | m/z = 741.91 |
| | (C₅₆H₃₉NO = 741.30) | | (C₅₆H₃₉NO = 741.30) |
| 408 | m/z = 689.84 | 422 | m/z = 679.80 |
| | (C₅₂H₃₅NO = 689.27) | | (C₅₀H₃₃NO₂ = 679.25) |
| 454 | m/z = 639.78 | 464 | m/z = 679.80 |
| | (C₄₈H₃₃NO = 639.26) | | (C₅₀H₃₃NO₂ = 679.25) |
| 482 | m/z = 589.72 | 571 | m/z = 721.95 |
| | (C₄₄H₃₁NO = 589.24) | | (C₅₃H₃₉NS = 721.28) |
| 611 | m/z = 721.95 | 635 | m/z = 757.98 |
| | (C₅₃H₃₉NS = 721.28) | | (C₅₆H₃₉NS = 757.28) |
| 698 | m/z = 711.93 | 725 | m/z = 690.97 |
| | (C₅₀H₃₃NS₂ = 711.21) | | (C₅₀H₁₀D₂₅NO = |
| | | | 690.43) |
| 741 | m/z = 712.97 | 742 | m/z = 683.93 |
| | (C₅₀H₁₆D₁₇NOS = | | (C₅₀H₁₇D₁₈NO = |
| | 712.33) | | 683.38) |

### EXPERIMENTAL EXAMPLES

### Experimental Example 1

### (1) Manufacture of Organic Light Emitting Device

A glass substrate thinly coated with ITO to have a thickness of 1,500 Å was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

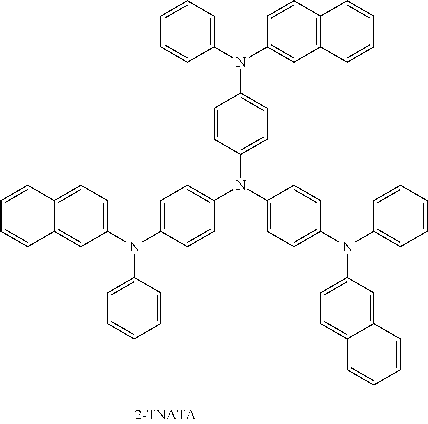 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 1000 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB.

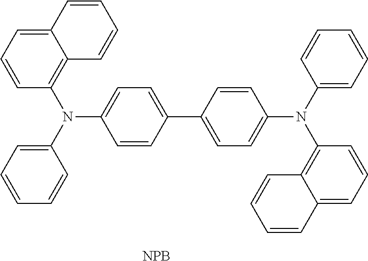 [SMILES: c1ccc(N(c2ccc(-c3ccc(N(c4ccccc4)c4cccc5ccccc45)cc3)cc2)c2cccc3ccccc23)cc1 ; "NPB"]

A light emitting layer was thermally vacuum deposited thereon as follows. The light emitting layer was deposited by depositing a compound 9-[4-(4,6-diphenyl-1,3,5-triazin-2-yl)phenyl]-9′-phenyl-3,3′-Bi-9H-carbazole as a host to have a thickness of 400 Å and doping the deposited layer with Ir(ppy)₃ as a green phosphorescent dopant by 7% of the thickness. Thereafter, bathocuproin (BCP) as a hole blocking layer was deposited to have a thickness of 60 Å, and E1 as an electron transport layer was deposited to have a thickness of 300 Å thereon.

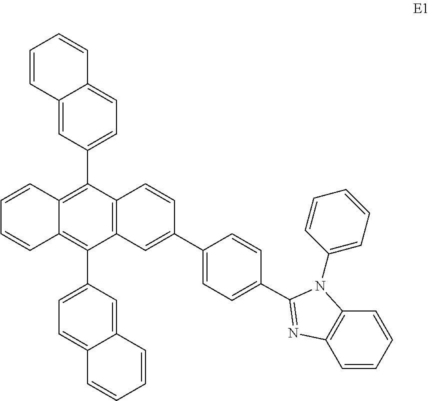 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

Finally, lithium fluoride (LiF) was deposited to have a thickness of 10 Å on the electron transport layer to form an electron injection layer, and then aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10⁻⁶ to 10⁻⁸ torr for each material, and used for the manufacture of OLED.

Thereafter, organic light emitting devices were manufactured in the same manner as in Experimental Example 1, except that the compounds described in the following Table 5 were used instead of Compound NPB used when the hole transport layer was formed in Experimental Example 1.

### (2) Driving Voltage and Light Emitting Efficiency of Organic Light Emitting Device

For the organic light emitting device manufactured as described above, electroluminescence (EL) characteristics were measured by M7000 manufactured by McScience Inc., and based on the measurement result thereof, T₉₅ (unit: h) was measured by a service life measurement equipment (M6000) manufactured by McScience Inc., when the reference luminance was 6,000 cd/m².

Characteristics of the organic electroluminescence device of the present invention are as shown in the following Table 5.

TABLE 5

| | | Driving | Light emitting | Service |
|---|---|---|---|---|
| | | voltage | efficiency | life |
| | Compound | (V) | (cd/A) | (T95) |
| Example 1 | 003 | 4.17 | 120.45 | 132 |
| Example 2 | 010 | 4.31 | 118.44 | 121 |
| Example 3 | 014 | 4.16 | 123.94 | 139 |
| Example 4 | 015 | 4.22 | 122.11 | 144 |
| Example 5 | 023 | 4.14 | 114.53 | 140 |
| Example 6 | 030 | 4.09 | 116.44 | 131 |
| Example 7 | 035 | 4.10 | 117.32 | 125 |
| Example 8 | 042 | 4.13 | 115.91 | 136 |
| Example 9 | 048 | 4.25 | 120.05 | 136 |
| Example 10 | 058 | 4.05 | 117.38 | 141 |
| Example 11 | 063 | 4.01 | 118.35 | 142 |
| Example 12 | 082 | 4.12 | 120.35 | 139 |
| Example 13 | 083 | 3.99 | 118.21 | 125 |
| Example 14 | 097 | 3.96 | 114.41 | 140 |
| Example 15 | 110 | 3.95 | 121.17 | 138 |
| Example 16 | 117 | 4.07 | 120.11 | 130 |
| Example 17 | 127 | 4.17 | 121.19 | 128 |
| Example 18 | 135 | 4.01 | 120.10 | 125 |
| Example 19 | 155 | 3.94 | 115.74 | 132 |
| Example 20 | 175 | 4.03 | 119.76 | 122 |
| Example 21 | 203 | 4.11 | 116.33 | 135 |
| Example 22 | 204 | 4.06 | 115.96 | 136 |
| Example 23 | 236 | 3.99 | 121.31 | 142 |
| Example 24 | 250 | 4.22 | 120.52 | 137 |
| Example 25 | 260 | 4.15 | 120.21 | 131 |
| Example 26 | 273 | 4.17 | 120.46 | 130 |
| Example 27 | 284 | 4.05 | 121.67 | 132 |
| Example 28 | 290 | 4.09 | 120.75 | 124 |
| Example 29 | 293 | 3.96 | 116.45 | 136 |
| Example 30 | 323 | 4.12 | 115.35 | 131 |
| Example 31 | 355 | 3.97 | 113.15 | 134 |
| Example 32 | 397 | 4.16 | 121.19 | 141 |
| Example 33 | 408 | 4.07 | 116.54 | 143 |
| Example 34 | 422 | 4.10 | 114.85 | 131 |
| Example 35 | 454 | 3.94 | 118.21 | 133 |
| Example 36 | 464 | 4.18 | 120.14 | 132 |
| Example 37 | 482 | 4.07 | 117.51 | 135 |
| Example 38 | 571 | 4.06 | 120.36 | 126 |
| Example 39 | 611 | 4.08 | 115.35 | 124 |
| Example 40 | 635 | 3.95 | 117.38 | 142 |
| Example 41 | 698 | 4.11 | 118.11 | 123 |
| Example 42 | 741 | 4.13 | 118.78 | 168 |
| Example 43 | 742 | 4.11 | 119.62 | 172 |
| Comparative | NPB | 5.58 | 84.22 | 113 |
| Example 1 | | | | |
| Comparative | M1 | 5.24 | 96.27 | 95 |
| Example 2 | | | | |
| Comparative | M2 | 6.29 | 47.80 | 46 |
| Example 3 | | | | |
| Comparative | M3 | 6.87 | 45.47 | 39 |
| Example 4 | | | | |
| Comparative | M4 | 5.56 | 94.55 | 90 |
| Example 5 | | | | |
| Comparative | M5 | 5.13 | 95.49 | 63 |
| Example 6 | | | | |
| Comparative | M6 | 5.21 | 92.18 | 69 |
| Example 7 | | | | |

In this case, the hole transport compounds (Compounds M1 to M6) of Comparative Examples 2 to 7 are as follows.

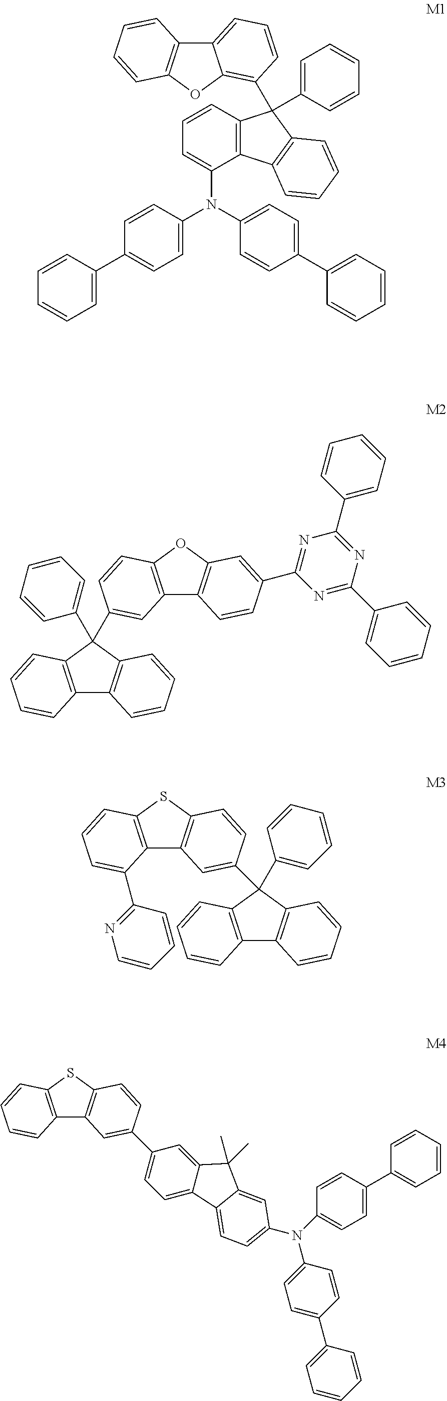 [SMILES: c1ccc(-c2ccc(N(c3ccc(-c4ccccc4)cc3)c3cccc4c3-c3ccccc3C4(c3ccccc3)c3cccc4c3oc3ccccc34)cc2)cc1 ; c1ccc(-c2nc(-c3ccccc3)nc(-c3ccc4c(c3)oc3ccc(C5(c6ccccc6)c6ccccc6-c6ccccc65)cc34)n2)cc1 ; c1ccc(C2(c3ccc4sc5cccc(-c6ccccn6)c5c4c3)c3ccccc3-c3ccccc32)cc1 ; CC1(C)c2cc(-c3ccc4sc5ccccc5c4c3)ccc2-c2ccc(N(c3ccc(-c4ccccc4)cc3)c3ccc(-c4ccccc4)cc3)cc21]

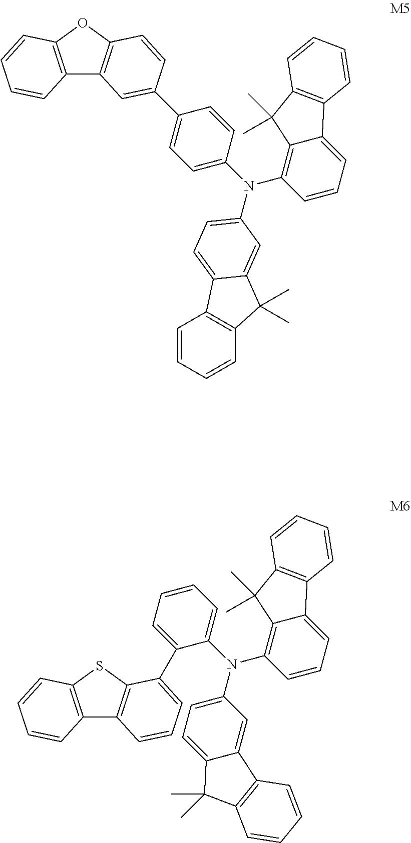 [SMILES: CC1(C)c2ccccc2-c2ccc(N(c3ccc(-c4ccc5oc6ccccc6c5c4)cc3)c3cccc4c3C(C)(C)c3ccccc3-4)cc21 ; CC1(C)c2ccccc2-c2cc(N(c3ccccc3-c3cccc4c3sc3ccccc34)c3cccc4c3C(C)(C)c3ccccc3-4)ccc21]

As can be seen from the results of Table 5, in Examples 1 to 43, which are organic light emitting devices using the compound represented by Chemical Formula 1 of the present invention as a hole transport layer material, the drive voltage was low, and the light emitting efficiency and service life were remarkably improved compared to Comparative Examples 1 to 7, which are organic light emitting devices that do not use the compound represented by Chemical Formula 1 of the present invention as a material for the hole transport layer.

When NPB, which is the hole transport layer material of Comparative Example 1 is compared to the compound represented by Chemical Formula 1 of the present invention, which is the hole transport layer material of Examples 1 to 43, the materials are similar in having an arylamine group, but there is a difference in that the compound represented by Chemical Formula 1 of the present invention has a structure in which an arylamine group is substituted with a substituent in which a fluorenyl group and a dibenzofuran group are linked. In this manner, the arylamine group is substituted with a substituent in which a fluorenyl group and a dibenzofuran group are linked, thereby suppressing the pi-pi stacking of the aromatic ring, and accordingly, it is determined that the driving voltage of the organic light emitting device is lowered to prevent a phenomenon in which the device characteristics deteriorate.

Further, when M1, which is the hole transport layer material of Comparative Example 2, is compared to the compound represented by Chemical Formula 1 of the present invention, which is the hole transport layer material of Examples 1 to 43, they are similar in having a substituent in which a fluorene group as an arylamine group and a dibenzofuran group are linked, but it can be confirmed that M1 of Comparative Example 2 and the compound represented by Chemical Formula 1 of the present invention have different substitution positions.

In addition, in the case of M2 to M6, which are the hole transport layer materials of Comparative Examples 3 to 7, it could be confirmed that when the compounds represented by Chemical Formula 1 of the present invention are compared, the structures of some substituents are similar, but the substitution position or the type of substituent was different, or the structure itself of the compound was different.

That is, since the amine group of the compound represented by Chemical Formula 1 of the present invention is substituted based on a dibenzofuran group, the hole mobility is higher than when an amine group substituted for a fluorene group is located as in M1 of Comparative Example 2, and due to an amine group substituted with a dibenzofuran group, the electron cloud distribution of the highest occupied molecular orbital (HOMO) of the present compound is distributed up to the dibenzofuran group, and thus the compound represented by Chemical Formula 1 of the present invention has an appropriate energy level, and accordingly, it is determined that the charge balance between holes and electrons in the light emitting layer was increased, and a device using the compound exhibited excellent results in terms of driving voltage and efficiency lifetime.

Likewise, even when compared to M2 to M6 used in Comparative Examples 3 to 7, the compound represented by Chemical Formula 1 of the present invention has high hole mobility and the charge balance between holes and electrons in the light emitting layer is increased, so that it is determined that excellent results are exhibited in terms of the driving voltage, efficiency and service life of the device using the compound represented by Chemical Formula 1 of the present invention.

### Experimental Example 2

### (1) Manufacture of Organic Light Emitting Device

Trichloroethylene, acetone, ethanol, and distilled water were each sequentially used to ultrasonically wash a transparent electrode ITO thin film obtained from glass for OLED (manufactured by Samsung-Corning Co., Ltd.) for 5 minutes, and then the ITO thin film was placed in isopropanol, stored, and then used. Next, the ITO substrate was disposed in a substrate folder of a vacuum deposition apparatus, and the following 4,4′,4″-tris(N,N-(2-naphthyl)-phenylamino)triphenyl amine (2-TNATA) was placed in a cell in the vacuum deposition apparatus.

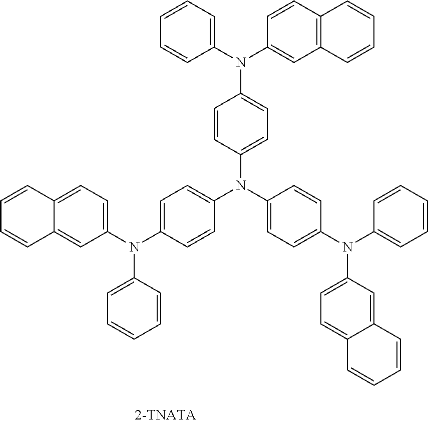 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 1000 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB.

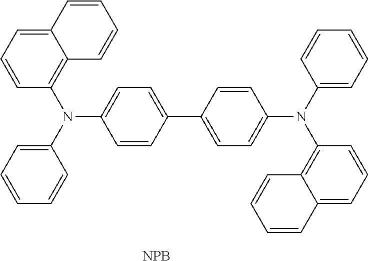 [SMILES: c1ccc(N(c2ccc(-c3ccc(N(c4ccccc4)c4cccc5ccccc45)cc3)cc2)c2cccc3ccccc23)cc1 ; "NPB"]

Subsequently, a compound having the following structural formula M1 as an electron blocking layer was deposited to have a thickness of 100 Å.

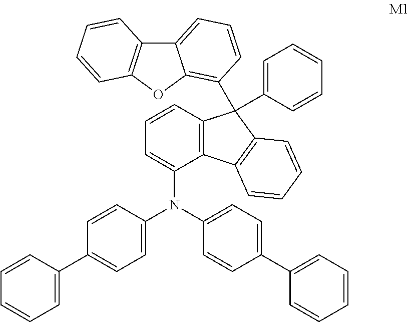 [SMILES: c1ccc(-c2ccc(N(c3ccc(-c4ccccc4)cc3)c3cccc4c3-c3ccccc3C4(c3ccccc3)c3cccc4c3oc3ccccc34)cc2)cc1]

A blue light emitting material having the following structure was deposited as a light emitting layer thereon. Specifically, a blue light emitting host material H1 was vacuum deposited to have a thickness of 300 Å on one cell in the vacuum deposition apparatus, and a blue light emitting dopant material D1 was vacuum deposited thereon in an amount of 5% based on the host material.

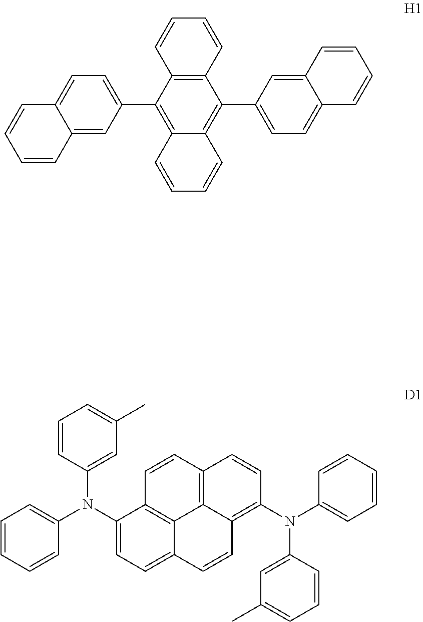

Subsequently, a compound having the following structural formula E1 as an electron transport layer was deposited to have a thickness of 300 Å.

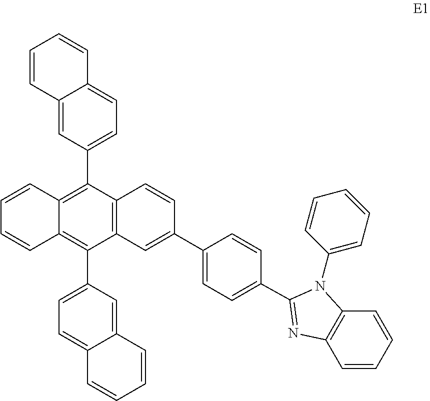 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An OLED device was manufactured by depositing lithium fluoride (LiF) as an electron injection layer to have a thickness of 10 Å and allowing the Al negative electrode to have a thickness of 1,000 Å. Meanwhile, all the organic compounds required for manufacturing an OLED device were subjected to vacuum sublimed purification under 10⁻⁶ to 10⁻⁸ torr for each material, and used for the manufacture of OLED.

Thereafter, organic light emitting devices were manufactured in the same manner as in Experimental Example 2, except that the compounds described in the following Table 6 were used instead of Compound M1 used when the electron blocking layer was formed in Experimental Example 2.

### (2) Driving Voltage and Light Emitting Efficiency of Organic Light Emitting Device

For the organic light emitting device manufactured as described above, electroluminescence (EL) characteristics were measured by M7000 manufactured by McScience Inc., and based on the measurement result thereof, T95 (unit: h) was measured by a service life measurement equipment (M6000) manufactured by McScience Inc., when the reference luminance was 6,000 cd/m².

Characteristics of the organic electroluminescence device of the present invention are as shown in the following Table 6.

TABLE 6

| | | Driving | Light emitting | Service |
|---|---|---|---|---|
| | | voltage | efficiency | life |
| | Compound | (V) | (cd/A) | (T95) |
| Example 44 | 003 | 5.42 | 6.87 | 52 |
| Example 45 | 011 | 5.38 | 6.78 | 49 |
| Example 46 | 023 | 5.37 | 6.81 | 56 |
| Example 47 | 035 | 5.49 | 7.12 | 57 |
| Example 48 | 048 | 5.44 | 6.85 | 50 |
| Example 49 | 058 | 5.41 | 6.54 | 60 |
| Example 50 | 069 | 5.35 | 7.11 | 63 |
| Example 51 | 083 | 5.35 | 6.56 | 54 |
| Example 52 | 084 | 5.47 | 6.61 | 54 |
| Example 53 | 117 | 5.37 | 6.46 | 62 |
| Example 54 | 138 | 5.33 | 6.53 | 54 |
| Example 55 | 155 | 5.31 | 6.83 | 51 |
| Example 56 | 203 | 5.42 | 6.77 | 61 |
| Example 57 | 204 | 5.35 | 6.70 | 53 |
| Example 58 | 260 | 5.40 | 6.83 | 55 |
| Example 59 | 290 | 5.39 | 6.72 | 53 |
| Example 60 | 323 | 5.36 | 6.69 | 55 |
| Example 61 | 408 | 5.32 | 6.97 | 57 |
| Example 62 | 571 | 5.39 | 6.84 | 59 |
| Example 63 | 635 | 5.25 | 6.88 | 52 |
| Example 64 | 725 | 5.45 | 6.64 | 78 |
| Comparative | M1 | 6.43 | 5.72 | 38 |
| Example 8 | | | | |
| Comparative | NPB | 6.60 | 5.35 | 33 |
| Example 9 | | | | |
| Comparative | M2 | 6.89 | 4.26 | 19 |
| Example 10 | | | | |
| Comparative | M3 | 6.84 | 4.52 | 22 |
| Example 11 | | | | |
| Comparative | M4 | 6.13 | 5.65 | 36 |
| Example 12 | | | | |
| Comparative | M5 | 6.25 | 5.78 | 39 |
| Example 13 | | | | |
| Comparative | M6 | 6.20 | 5.69 | 32 |
| Example 14 | | | | |

As can be seen from the results of Table 6, in Examples 44 to 64, which are organic light emitting devices using the compound represented by Chemical Formula 1 of the present invention as an electron blocking layer material, the drive voltage was low, and the light emitting efficiency and service life were remarkably improved compared to Comparative Examples 8 to 14, which are organic light emitting devices using M1 to M6 and the NPB compound as electron blocking layer materials.

In general, when electrons are not combined in the light emitting layer and pass through the hole transport layer and go to the positive electrode, there occurs a phenomenon in which the efficiency and service life of the organic light emitting device are reduced. In this case, when a compound having a high lowest unoccupied molecular orbital (LUMO) level is used as the electron blocking layer, the energy barrier of the electron blocking layer prevents the electrons from passing through the light emitting layer and going to the positive electrode, and as a result, it is possible to prevent the phenomenon in which the efficiency and service life of the organic light emitting device are reduced. That is, when a compound having a high lowest unoccupied molecular orbital (LUMO) level is used as the electron blocking layer, the probability that holes and electrons form excitons increases, and the exciton is highly likely to be emitted as light in the light emitting layer.

Therefore, it is determined that since the compound of the present invention has a higher LUMO level and a wider band gap than the compounds of Comparative Examples 8 to 14, when the compound of the present invention is used as an electron blocking layer of an organic light emitting device, the electron blocking ability is even better, and the holes and electrons form a charge balance to emit light inside the light emitting layer rather than at the interface of the hole transport layer, and thus the driving voltage, efficiency, and service life of a device using the compound of the present invention are excellent.

### EXPLANATION OF REFERENCE NUMERALS AND SYMBOLS

100: Substrate
200: Positive electrode
300: Organic material layer
301: Hole injection layer
302: Hole transport layer
303: Light emitting layer
304: Hole blocking layer
305: Electron transport layer
306: Electron injection layer
400: Negative electrode

1. A heterocyclic compound represented by the following Chemical Formula 1: 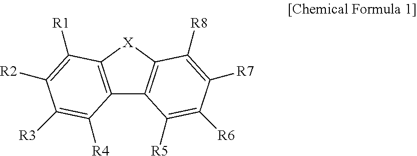
wherein, in Chemical Formula 1,
X is O; or S,
one of R1 to R8 is the following Chemical Formula A, one of the others is the following Chemical Formula B, and the others are the same as or different from each other, and are each independently selected from the group consisting of hydrogen; deuterium; a halogen group; a cyano group; a substituted or unsubstituted alkyl group having 1 to 60 carbon atoms; a substituted or unsubstituted aryl group having 6 to 60 carbon atoms; and a substituted or unsubstituted heteroaryl group having 2 to 60 carbon atoms, 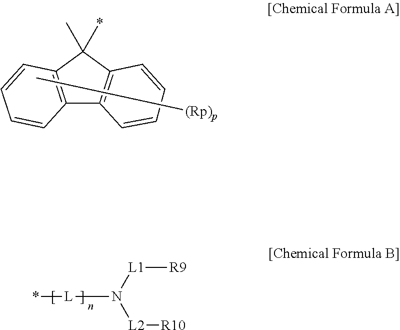
in Chemical Formulae A and B,
Rp is hydrogen; deuterium; a halogen group; a cyano group; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms, p is an integer from 0 to 8, and when p is 2 or higher, Rp's in the parenthesis are the same as or different from each other,
L, L1 and L2 are the same as or different from each other, and are each independently a direct bond; a substituted or unsubstituted arylene group having 6 to 60 carbon atoms; or a substituted or unsubstituted heteroarylene group having 2 to 60 carbon atoms,
R9 and R10 are the same as or different from each other, and are each independently a substituted or unsubstituted aryl group having 6 to 60 carbon atoms; or a substituted or unsubstituted heteroaryl group having 2 to 60 carbon atoms,
n is an integer from 0 to 3, and when n is 2 or higher, L's in the parenthesis are the same as or different from each other,
* is a position where Chemical Formula 1, Chemical Formula A and Chemical Formula B are each bonded, and
when R1 is Chemical Formula A, X is S.

2. The heterocyclic compound of claim 1, wherein Chemical Formula 1 is represented by any one of the following Chemical Formulae 2 to 5: 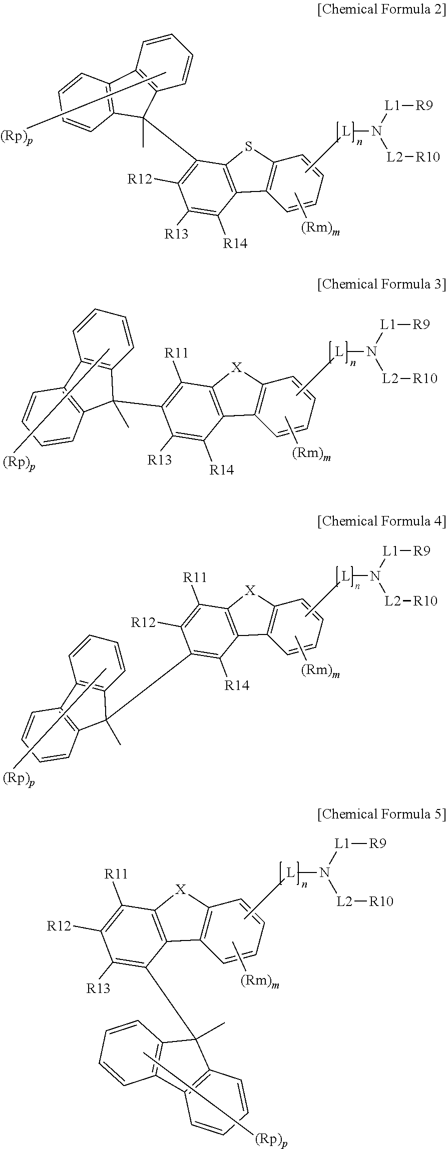
in Chemical Formulae 2 to 5,
R11 to R14 and Rm are the same as or different from each other, and are each independently hydrogen; deuterium; a halogen group; a cyano group; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms, m is an integer from 0 to 3, and when m is 2 or higher, Rm's in the parenthesis are the same as or different from each other, and
the definitions of X, L, L1, L2, R9, R10, Rp, n and p are the same as those in Chemical Formula 1.

3. The heterocyclic compound of claim 1, wherein Chemical Formula 1 is represented by any one of the following Chemical Formulae 6 to 9: 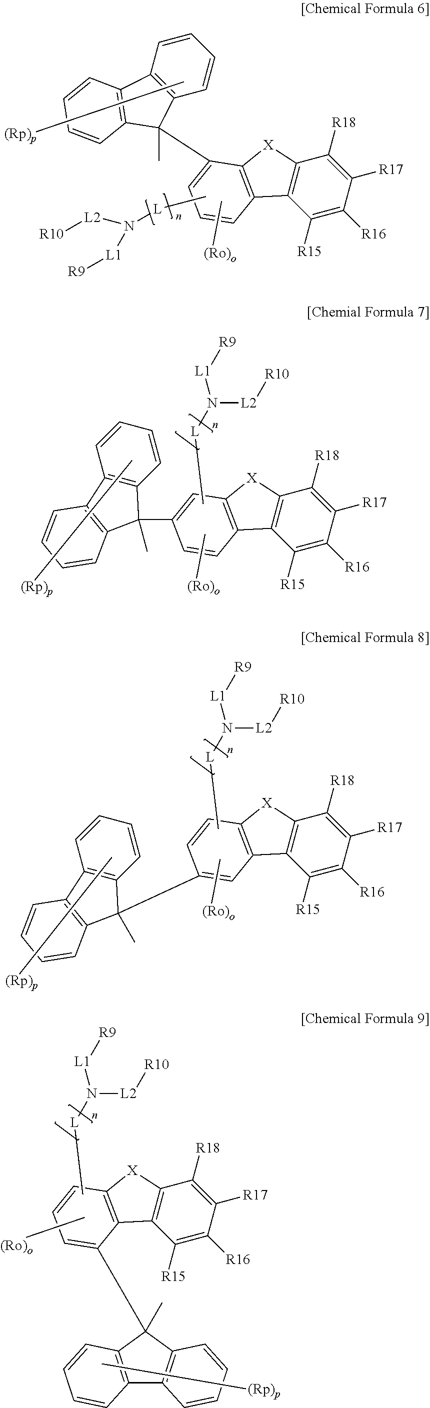
in Chemical Formulae 6 to 9,
R15 to R18 and Ro are the same as or different from each other, and are each independently hydrogen; deuterium; a halogen group; a cyano group; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms, o is an integer from 0 to 3, and when o is 2 or higher, Ro's in the parenthesis are the same as or different from each other, and
the definitions of X, L, L1, L2, R9, R10, Rp, n and p are the same as those in Chemical Formula 1.

4. The heterocyclic compound of claim 1, wherein one of R1 to R8 is Chemical Formula A, one of the others is Chemical Formula B, and the others are hydrogen; or deuterium.

5. The heterocyclic compound of claim 1, wherein a deuterium content of the compound represented by Chemical Formula 1 is 10% or more and 100% or less.

6. The heterocyclic compound of claim 1, wherein R9 and R10 are the same as or different from each other, and are each independently a substituted or unsubstituted aryl group having 6 to 40 carbon atoms; or a substituted or unsubstituted heteroaryl group having 2 to 40 carbon atoms.

7. The heterocyclic compound of claim 1, wherein Chemical Formula 1 is represented by any one of the following compounds:

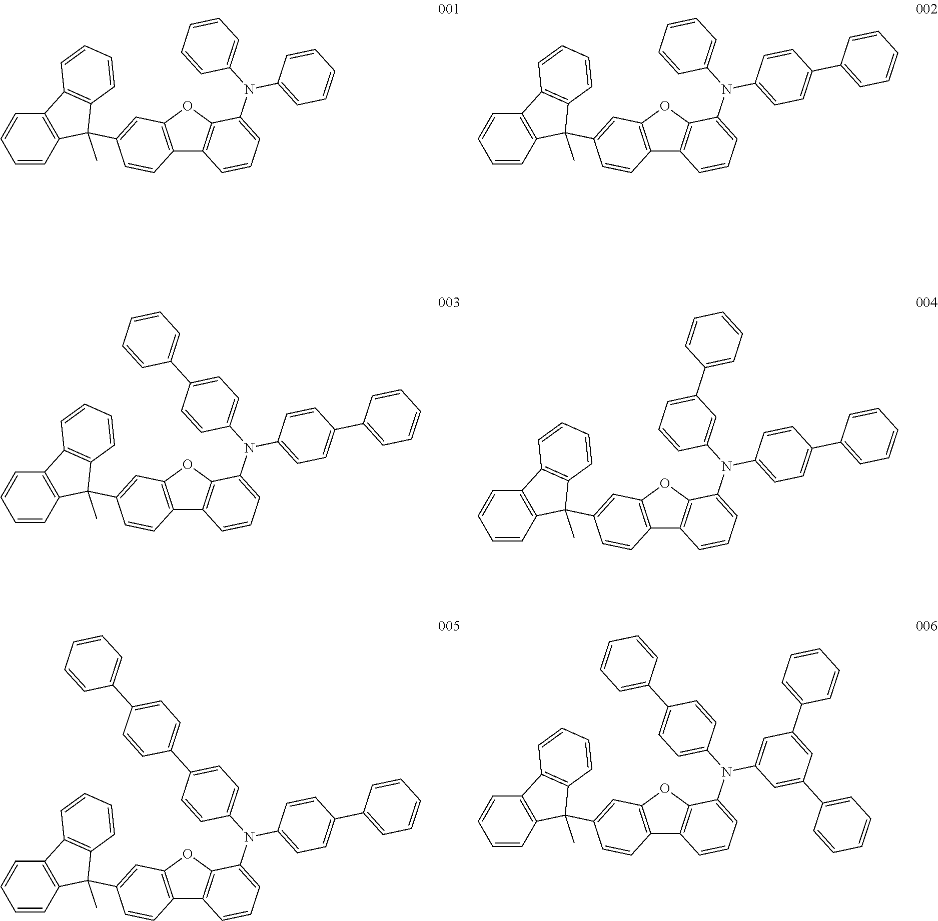

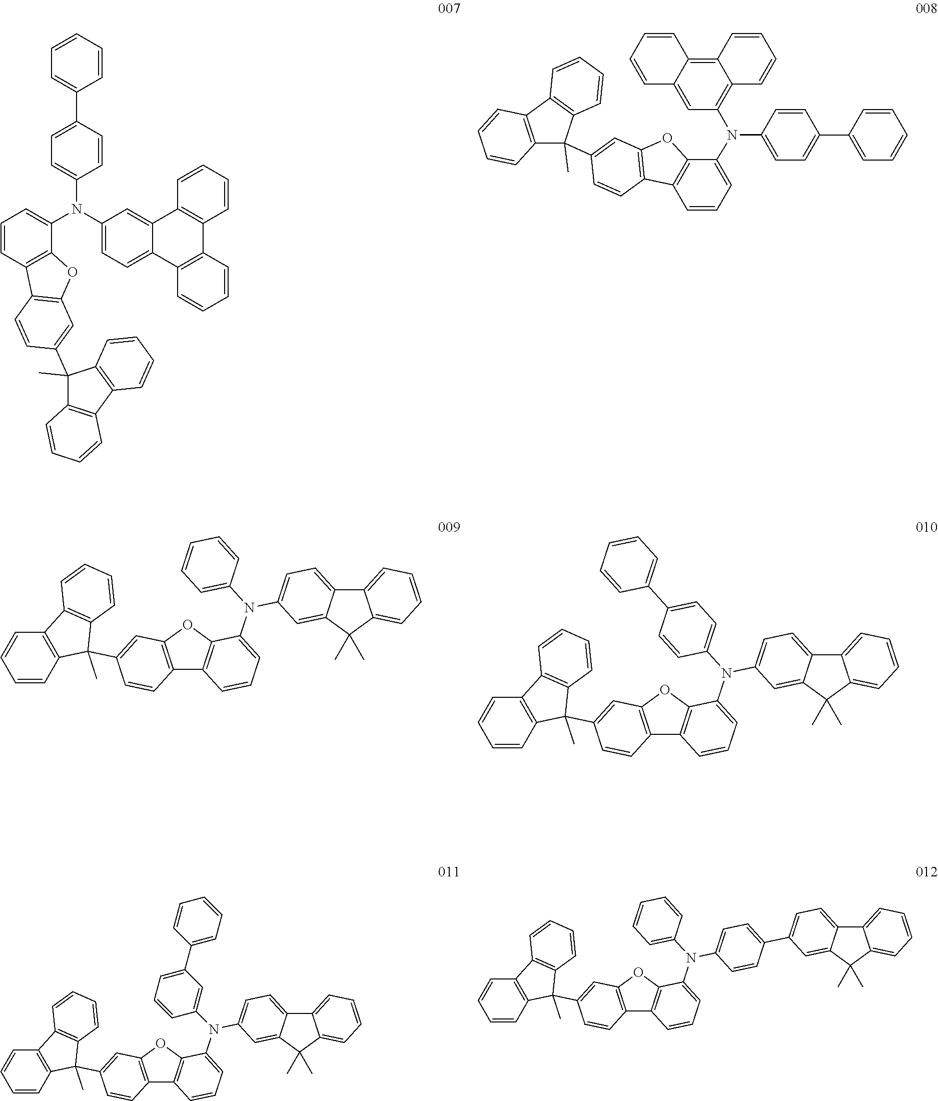

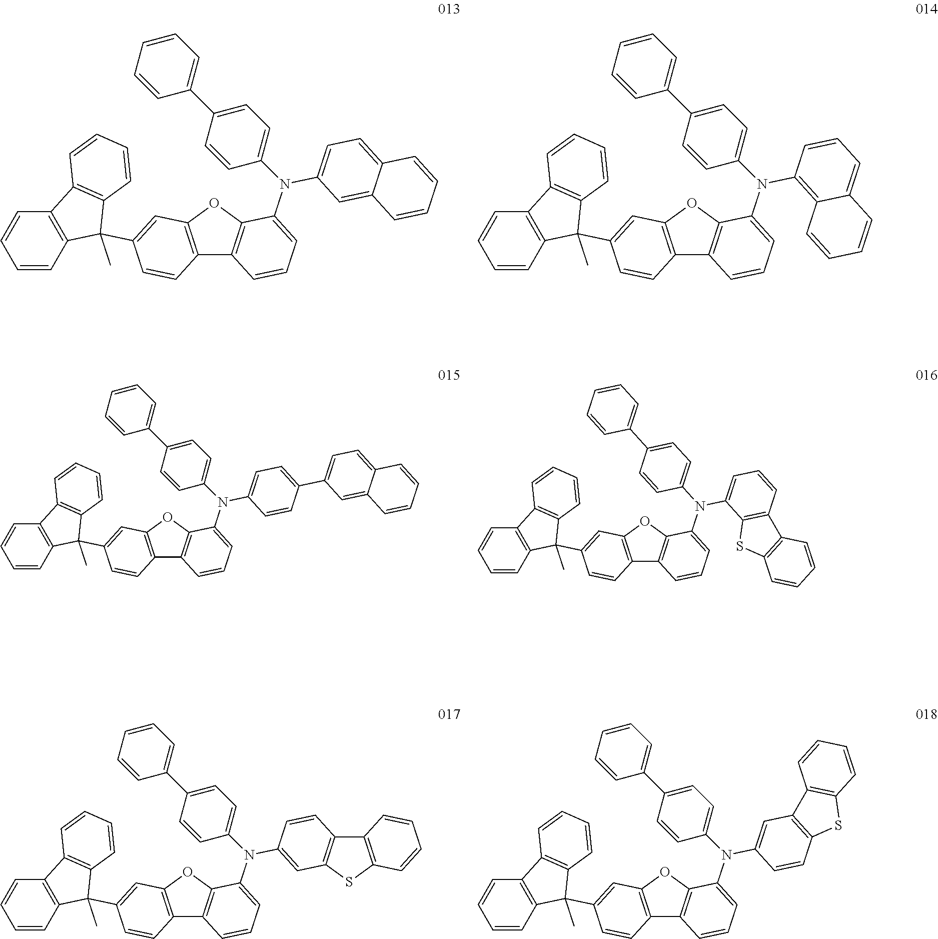

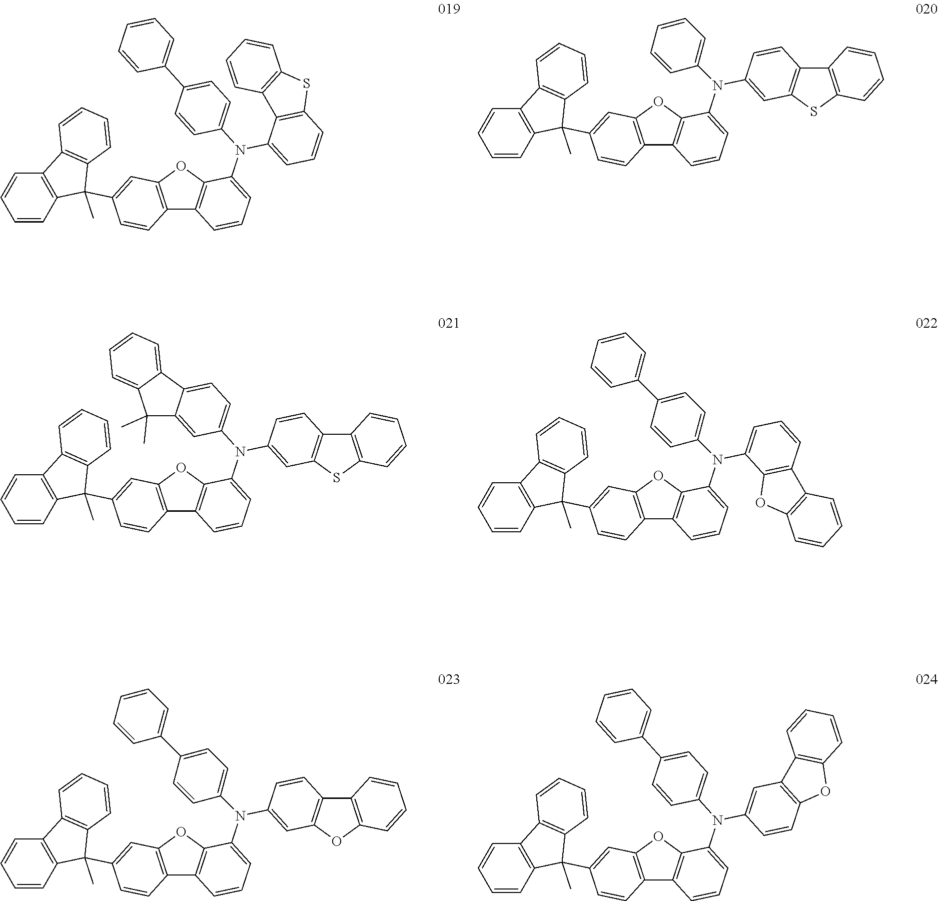

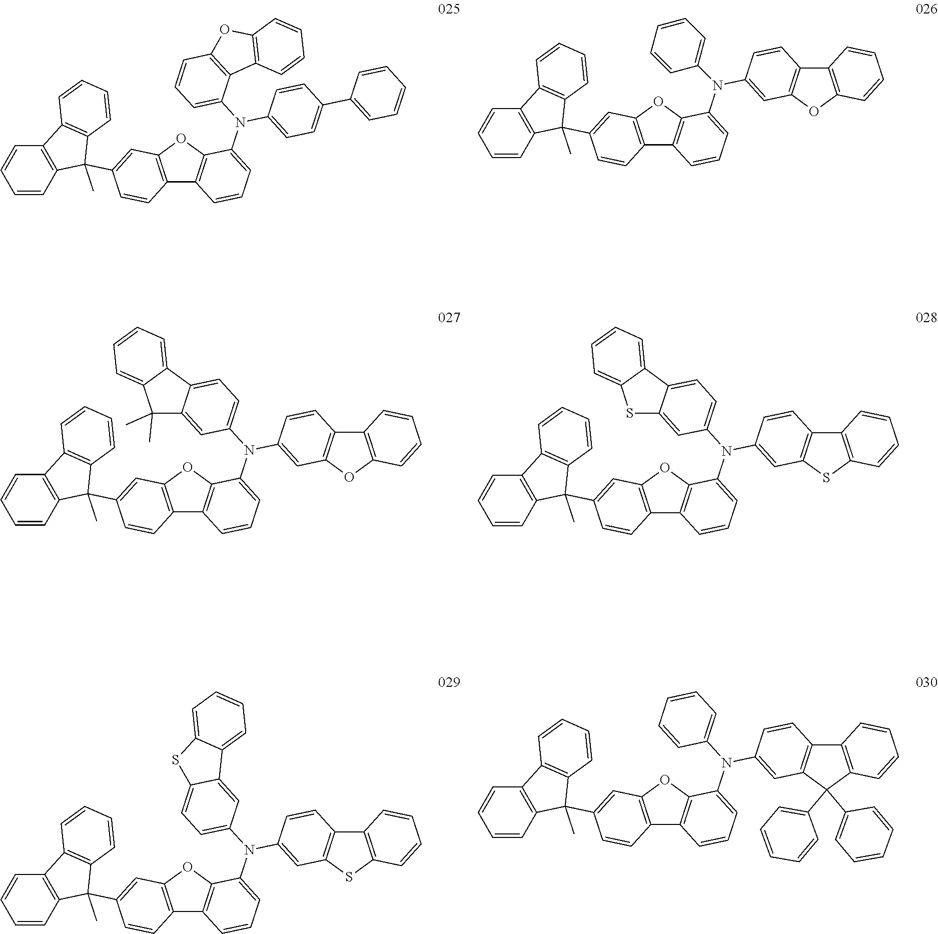

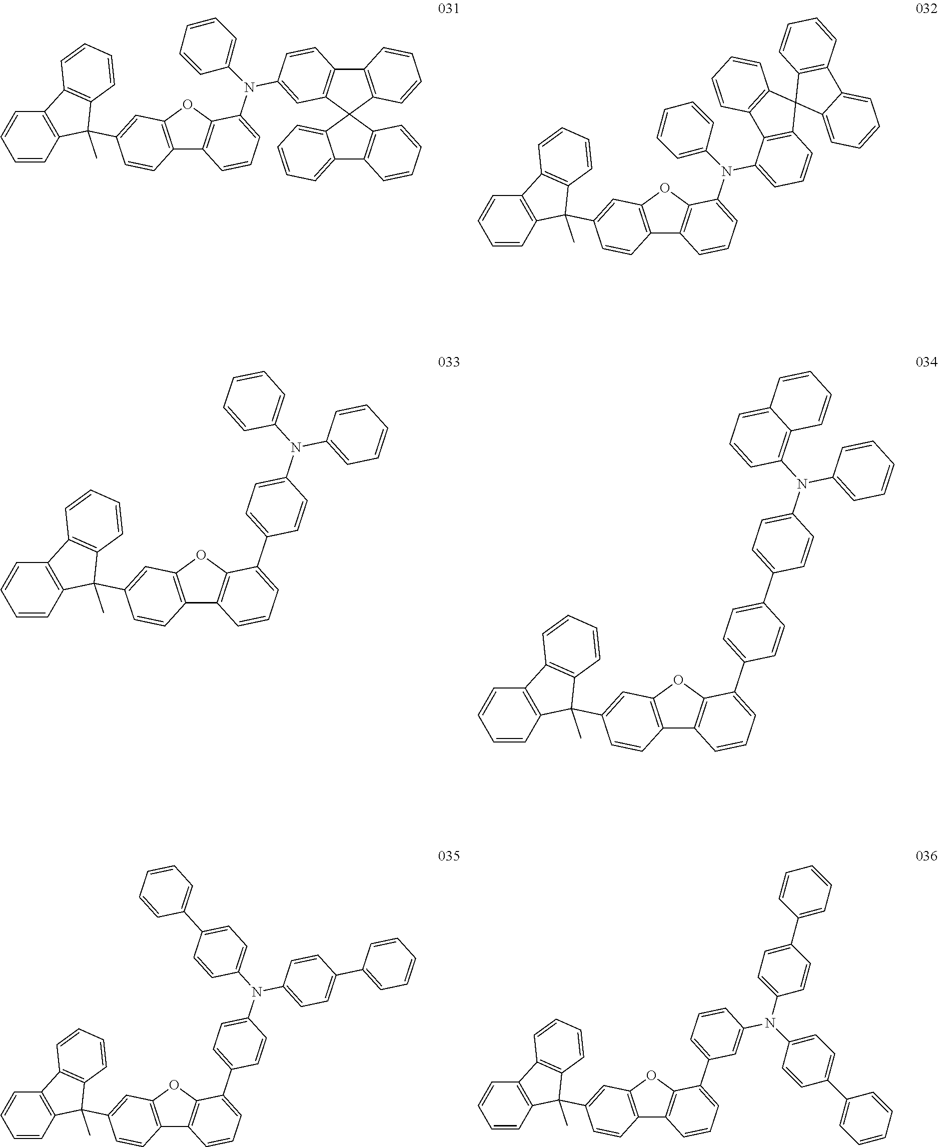

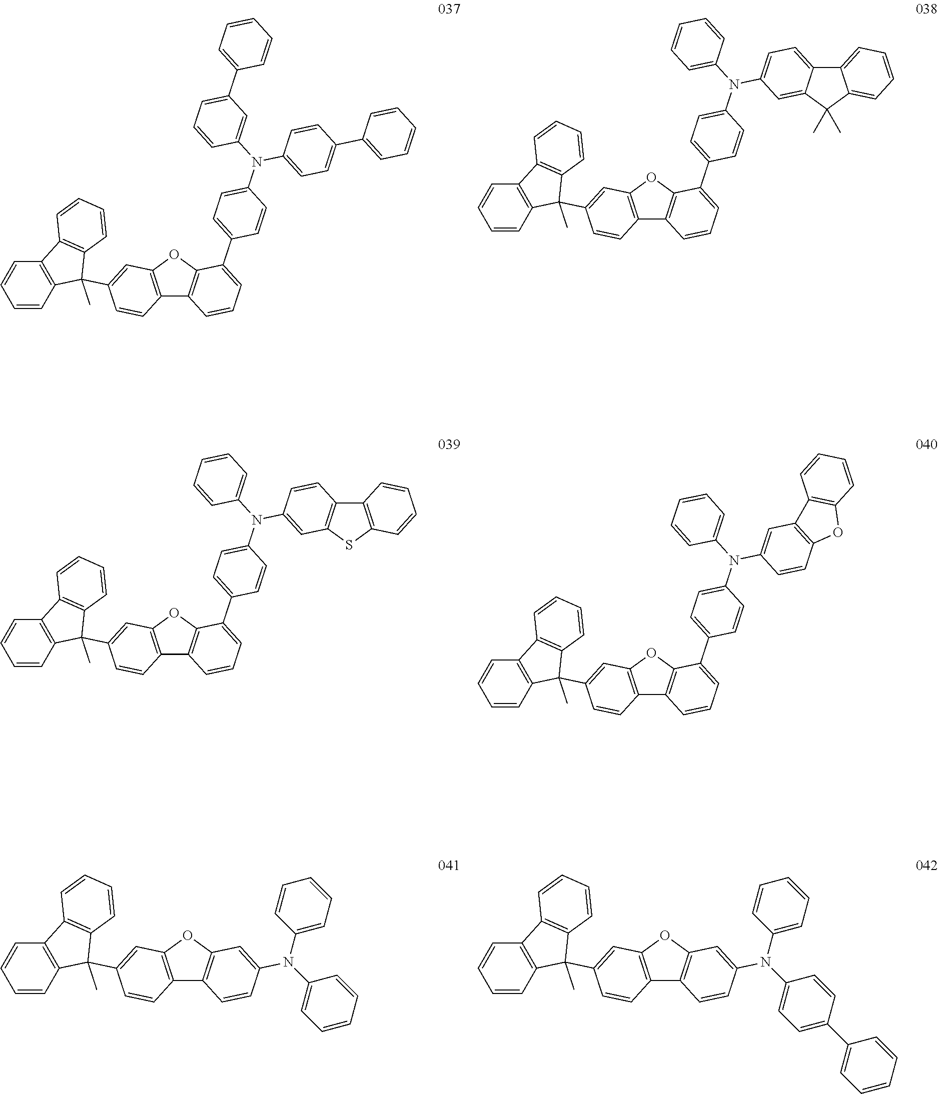

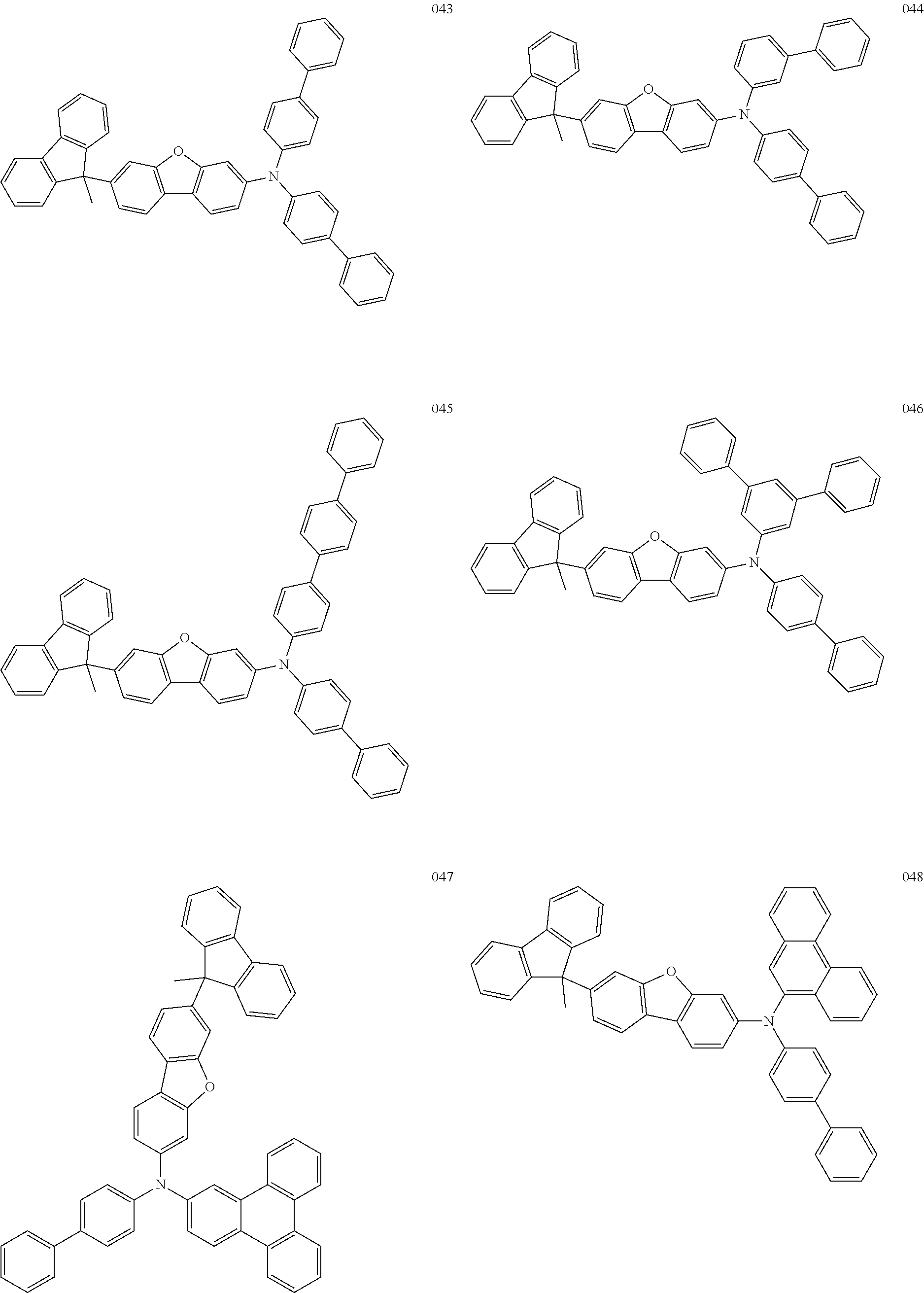

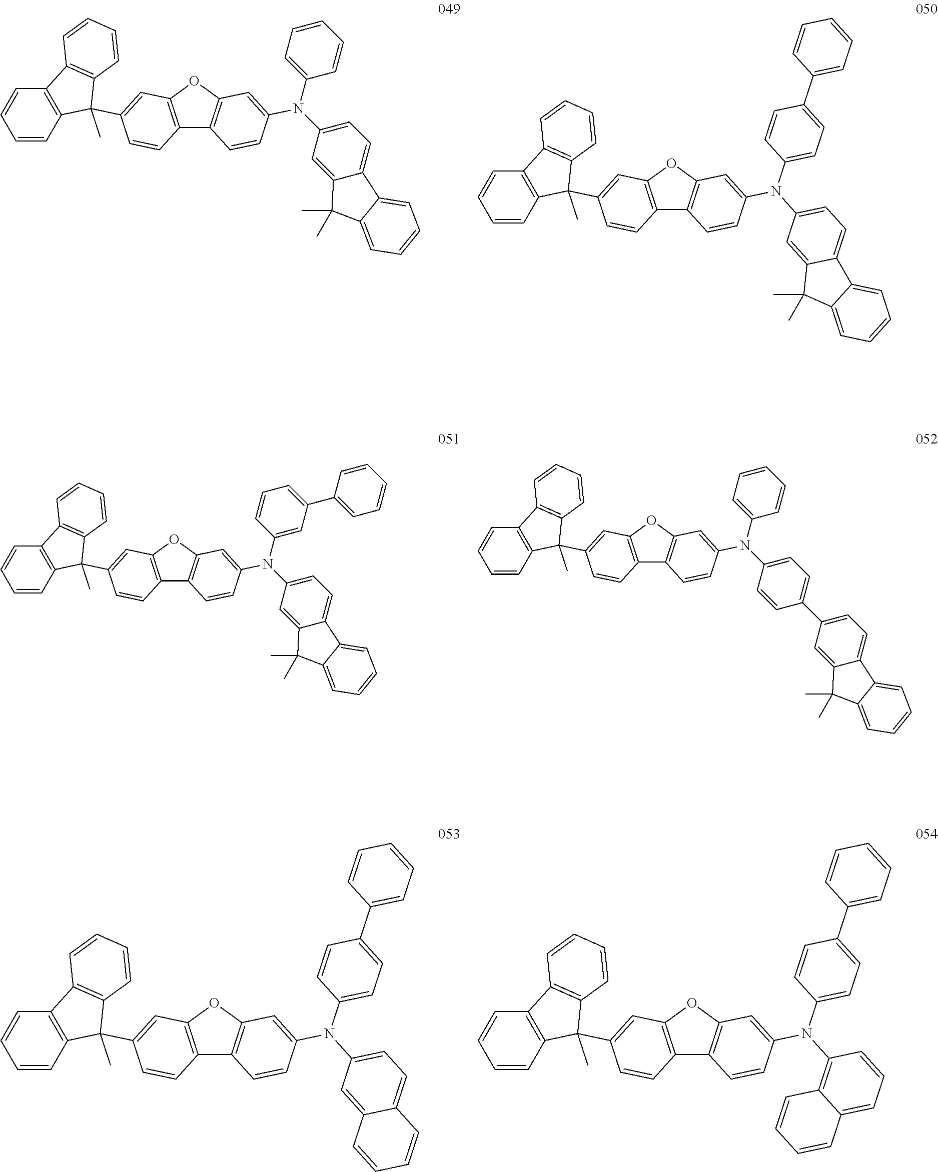

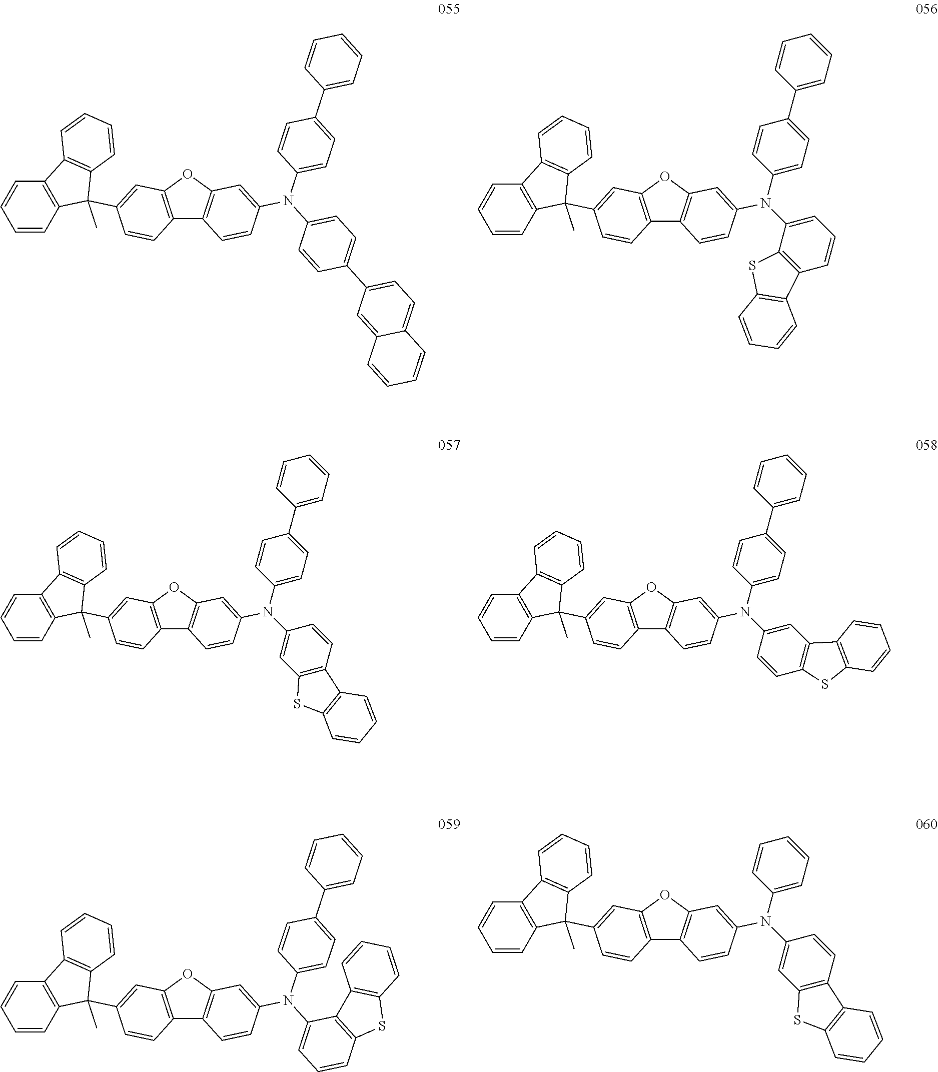

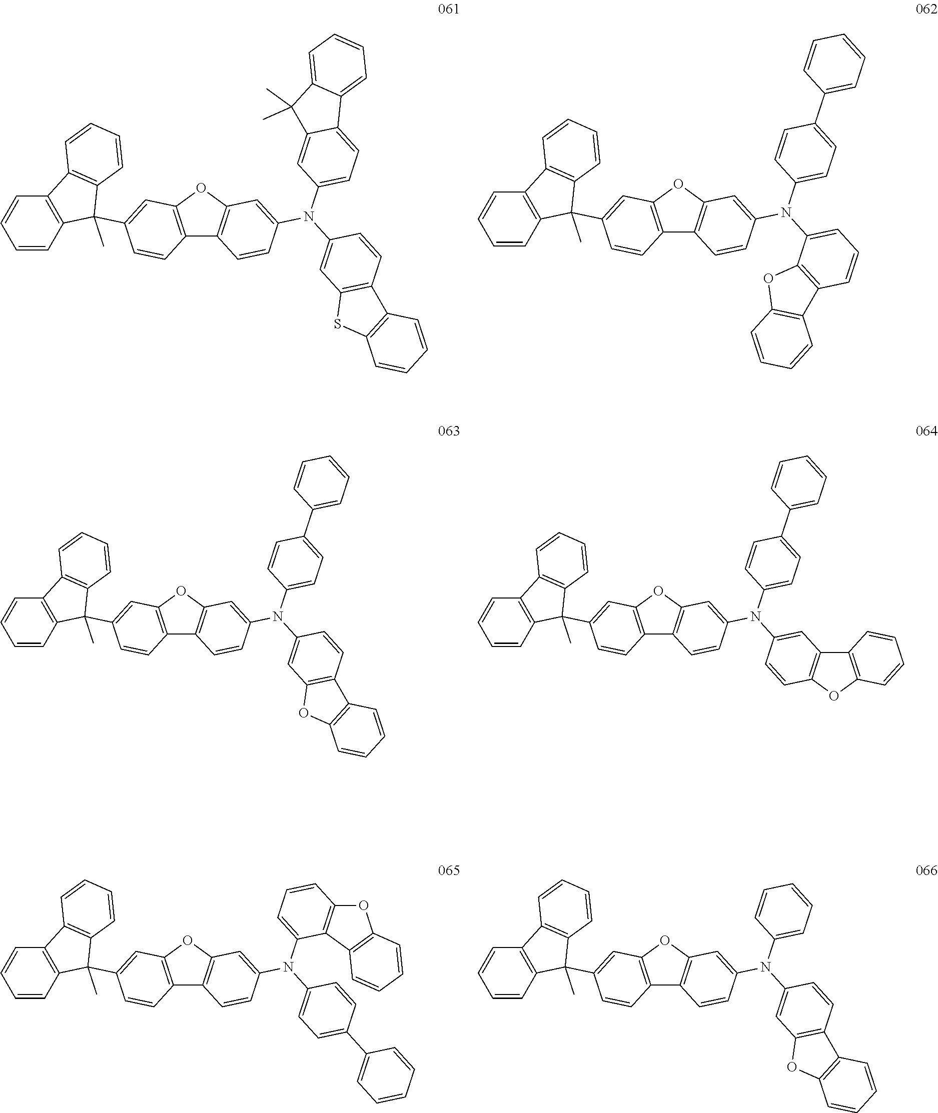

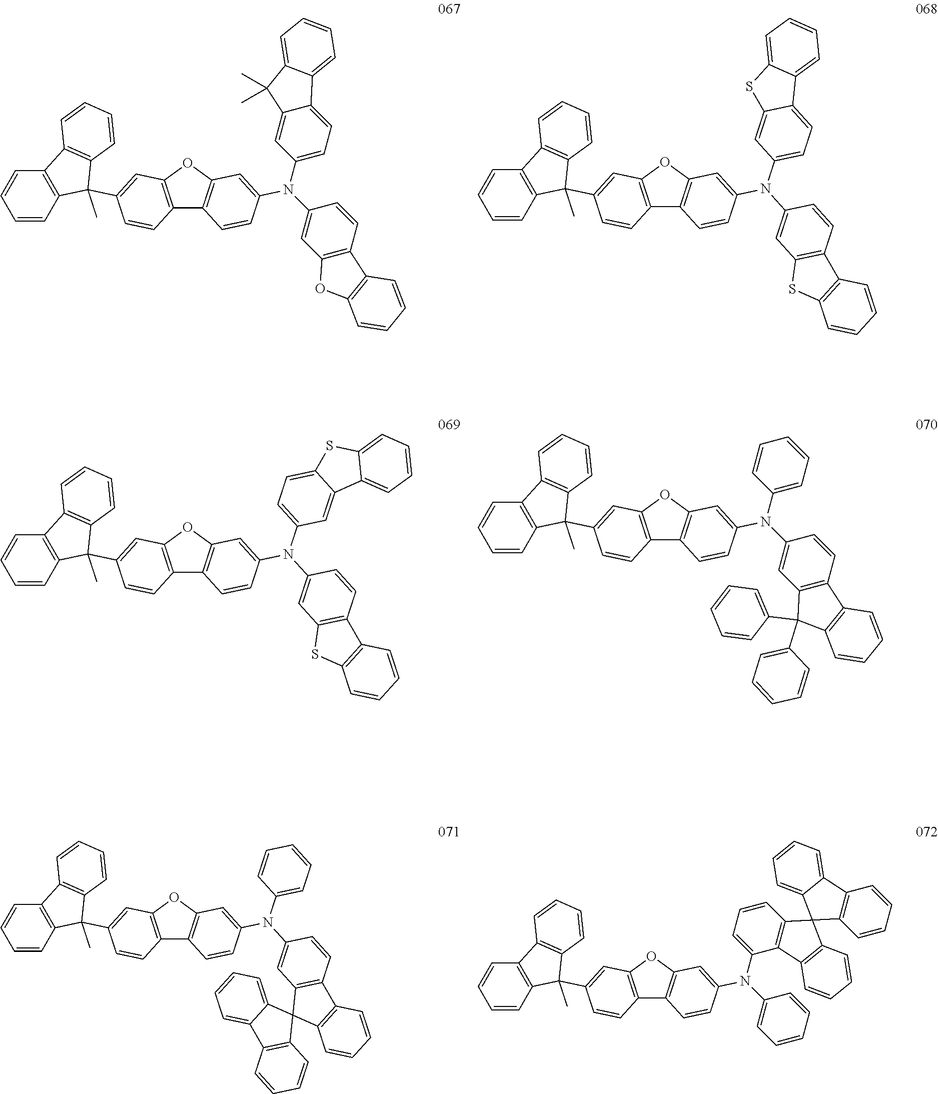

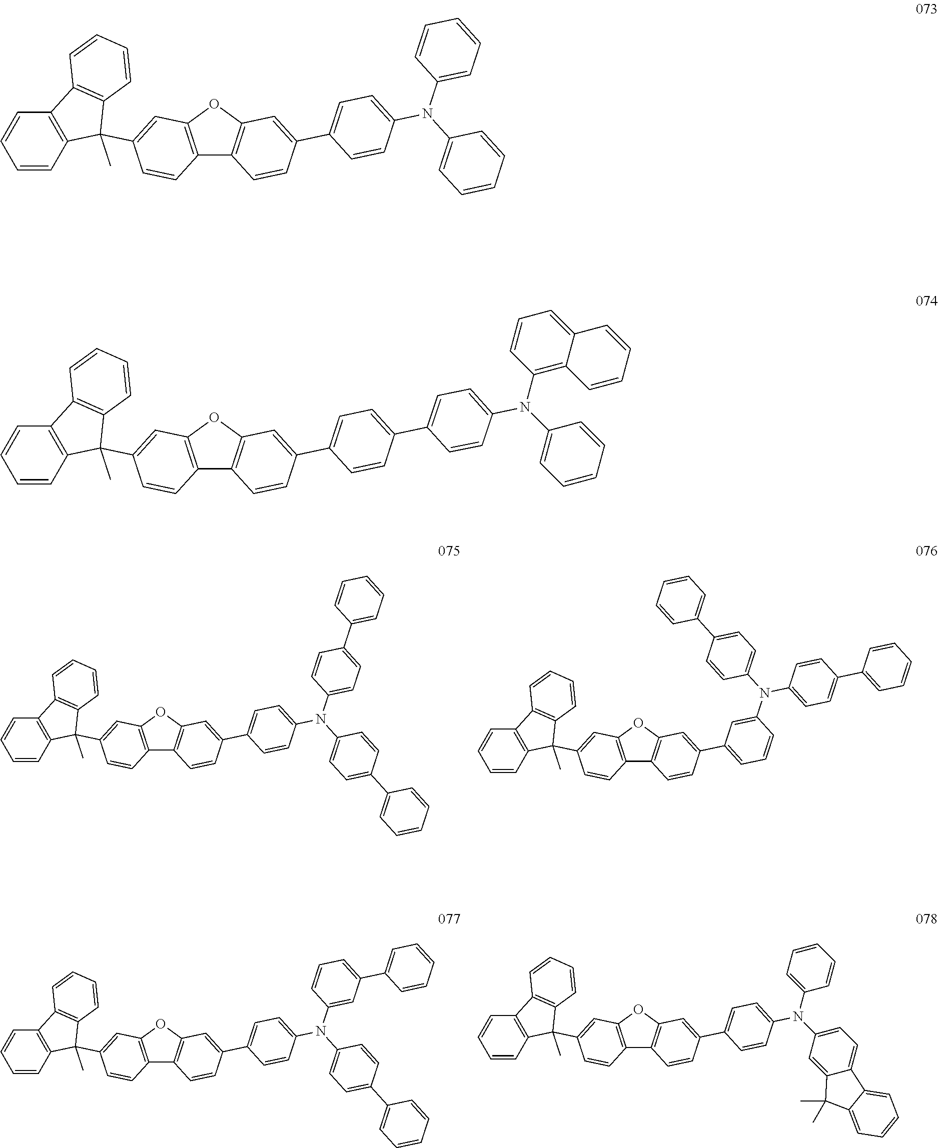

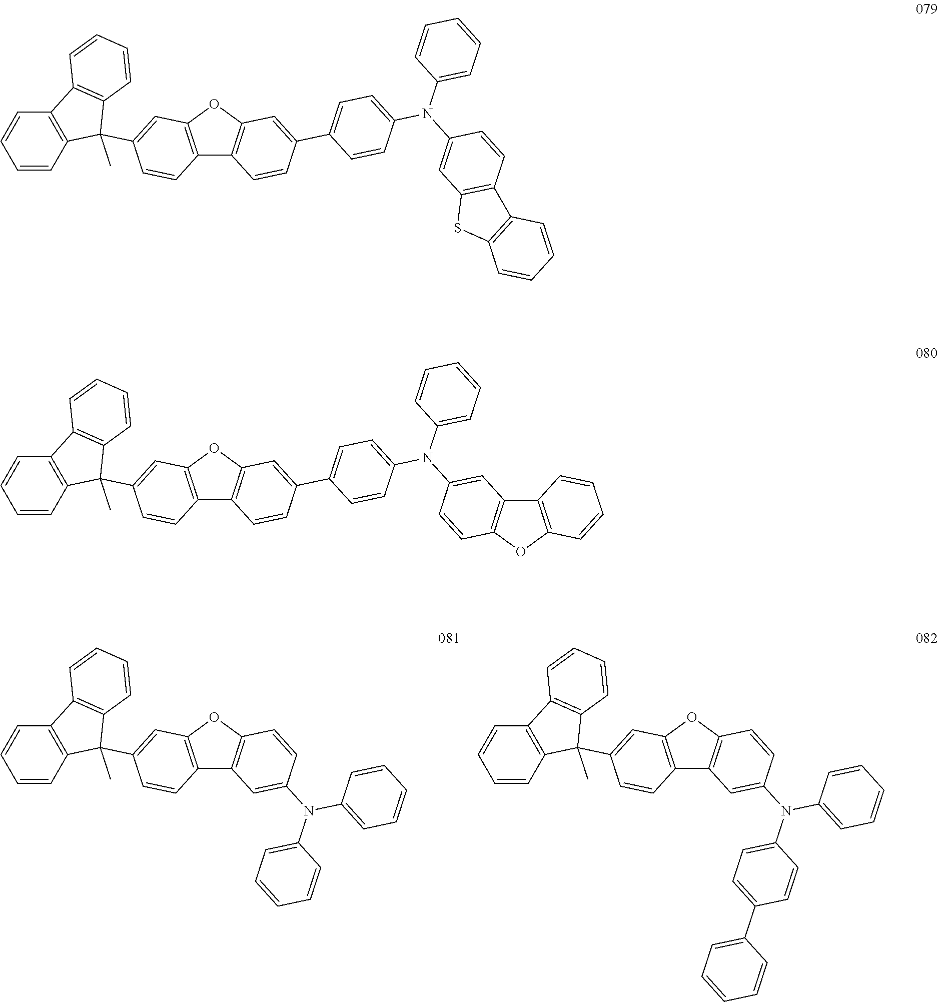 [SMILES: CC1(c2ccc3c(c2)oc2cc(-c4ccc(N(c5ccccc5)c5ccc6c(c5)sc5ccccc56)cc4)ccc23)c2ccccc2-c2ccccc21 ; CC1(c2ccc3c(c2)oc2cc(-c4ccc(N(c5ccccc5)c5ccc6oc7ccccc7c6c5)cc4)ccc23)c2ccccc2-c2ccccc21 ; CC1(c2ccc3c(c2)oc2ccc(N(c4ccccc4)c4ccccc4)cc23)c2ccccc2-c2ccccc21 ; CC1(c2ccc3c(c2)oc2ccc(N(c4ccccc4)c4ccc(-c5ccccc5)cc4)cc23)c2ccccc2-c2ccccc21]

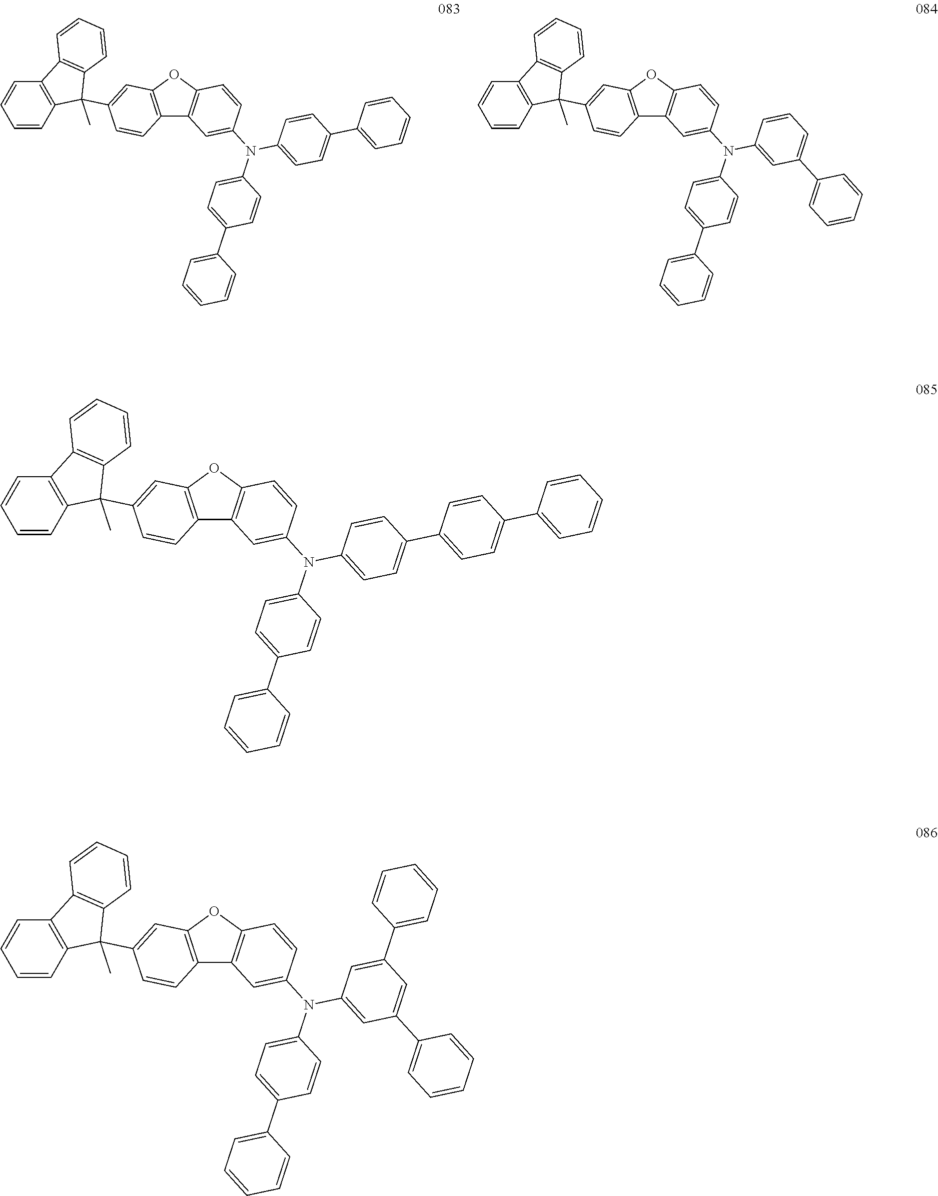 [SMILES: CC1(c2ccc3c(c2)oc2ccc(N(c4ccc(-c5ccccc5)cc4)c4ccc(-c5ccccc5)cc4)cc23)c2ccccc2-c2ccccc21 ; CC1(c2ccc3c(c2)oc2ccc(N(c4ccc(-c5ccccc5)cc4)c4cccc(-c5ccccc5)c4)cc23)c2ccccc2-c2ccccc21 ; CC1(c2ccc3c(c2)oc2ccc(N(c4ccc(-c5ccccc5)cc4)c4ccc(-c5ccc(-c6ccccc6)cc5)cc4)cc23)c2ccccc2-c2ccccc21 ; CC1(c2ccc3c(c2)oc2ccc(N(c4ccc(-c5ccccc5)cc4)c4cc(-c5ccccc5)cc(-c5ccccc5)c4)cc23)c2ccccc2-c2ccccc21]

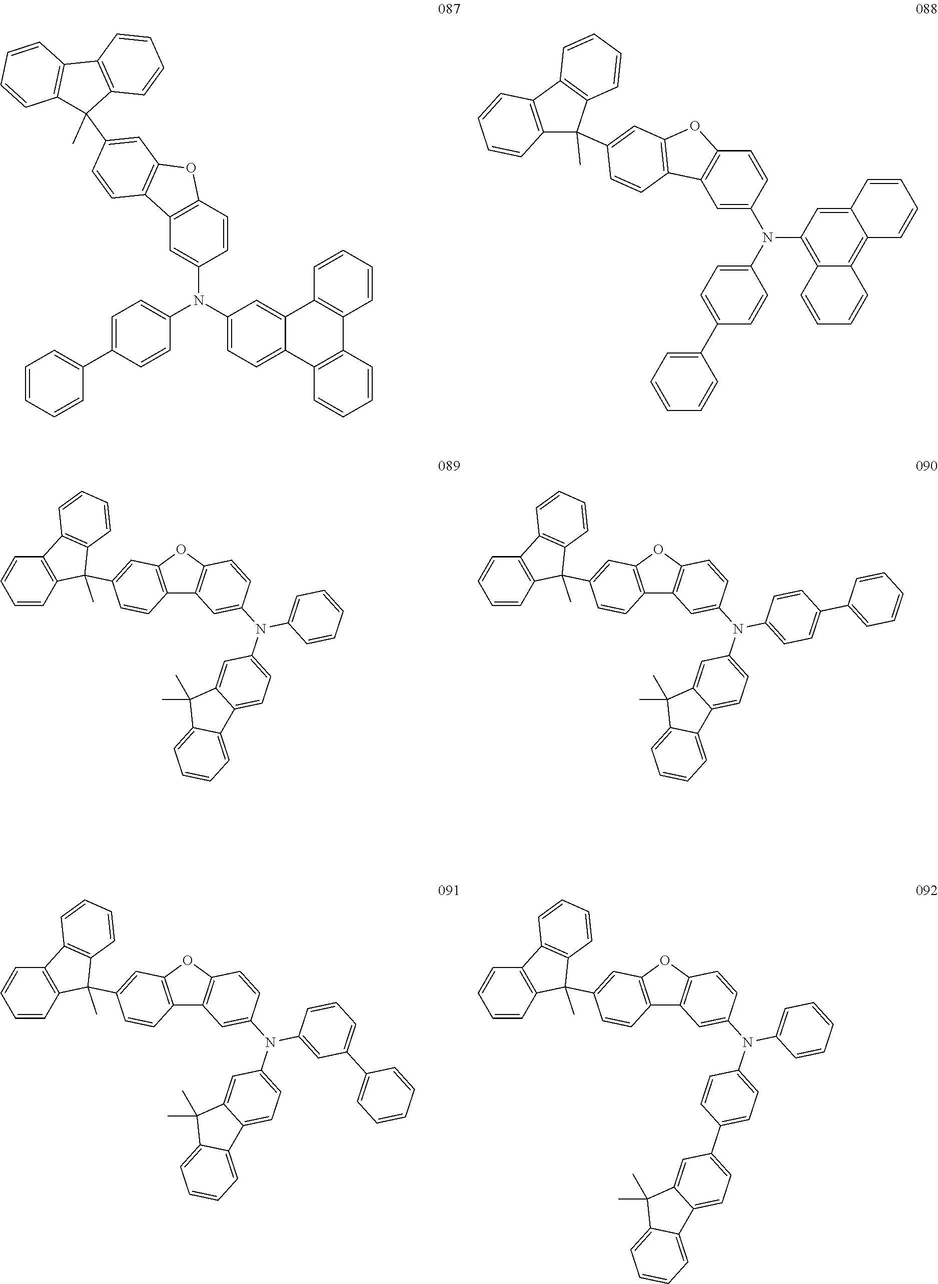

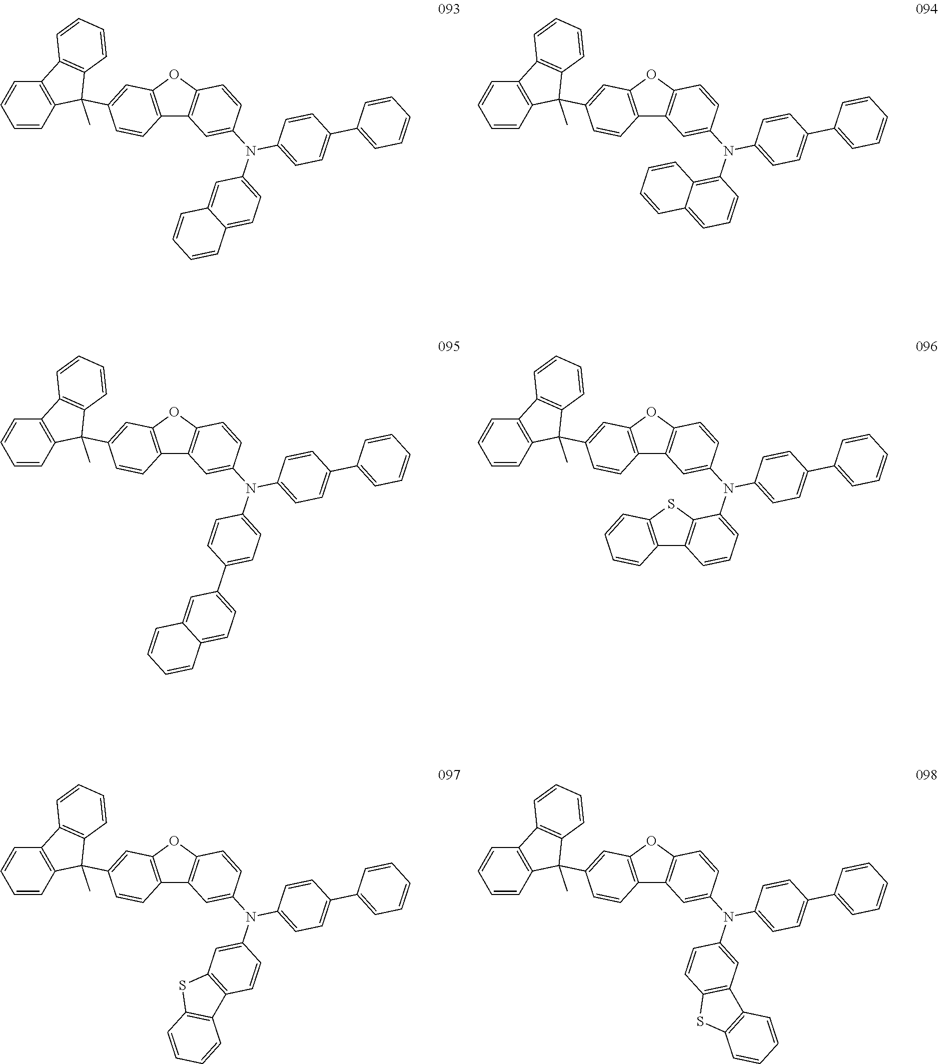

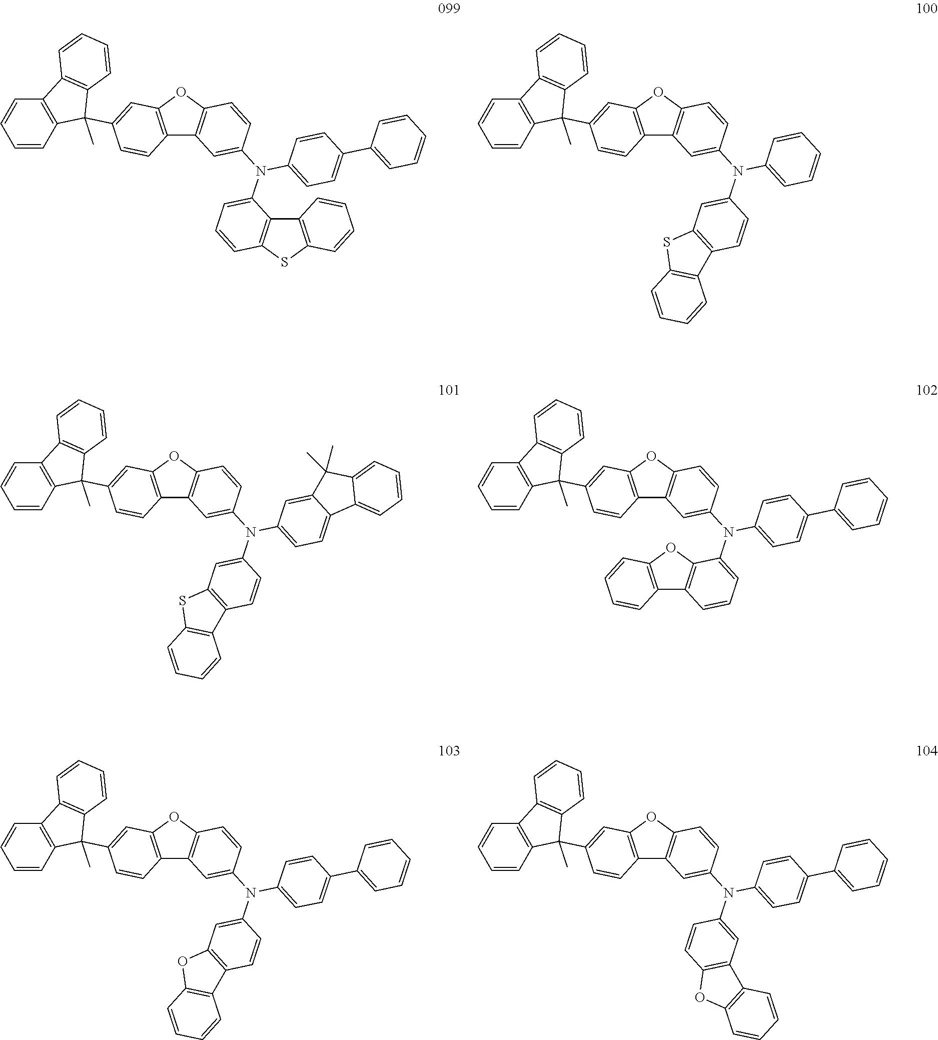

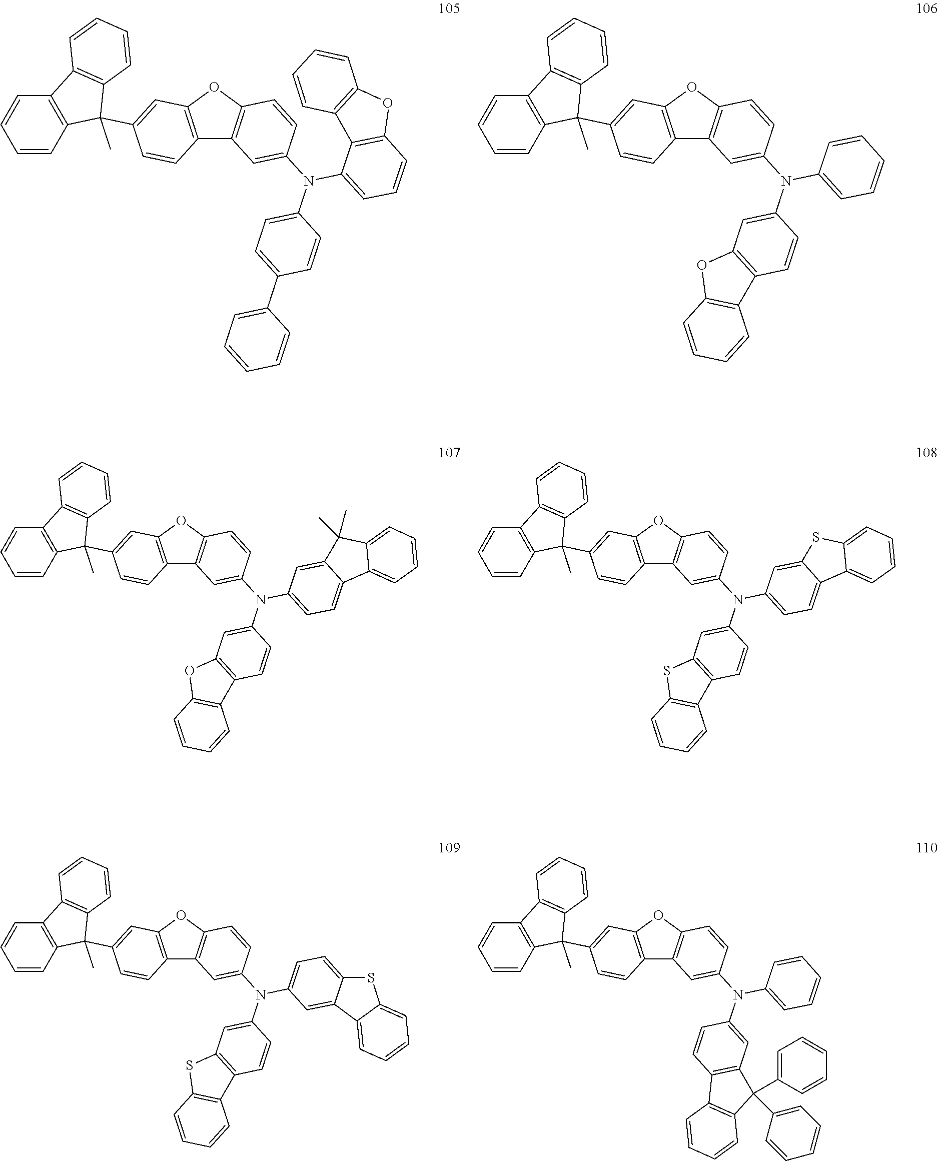

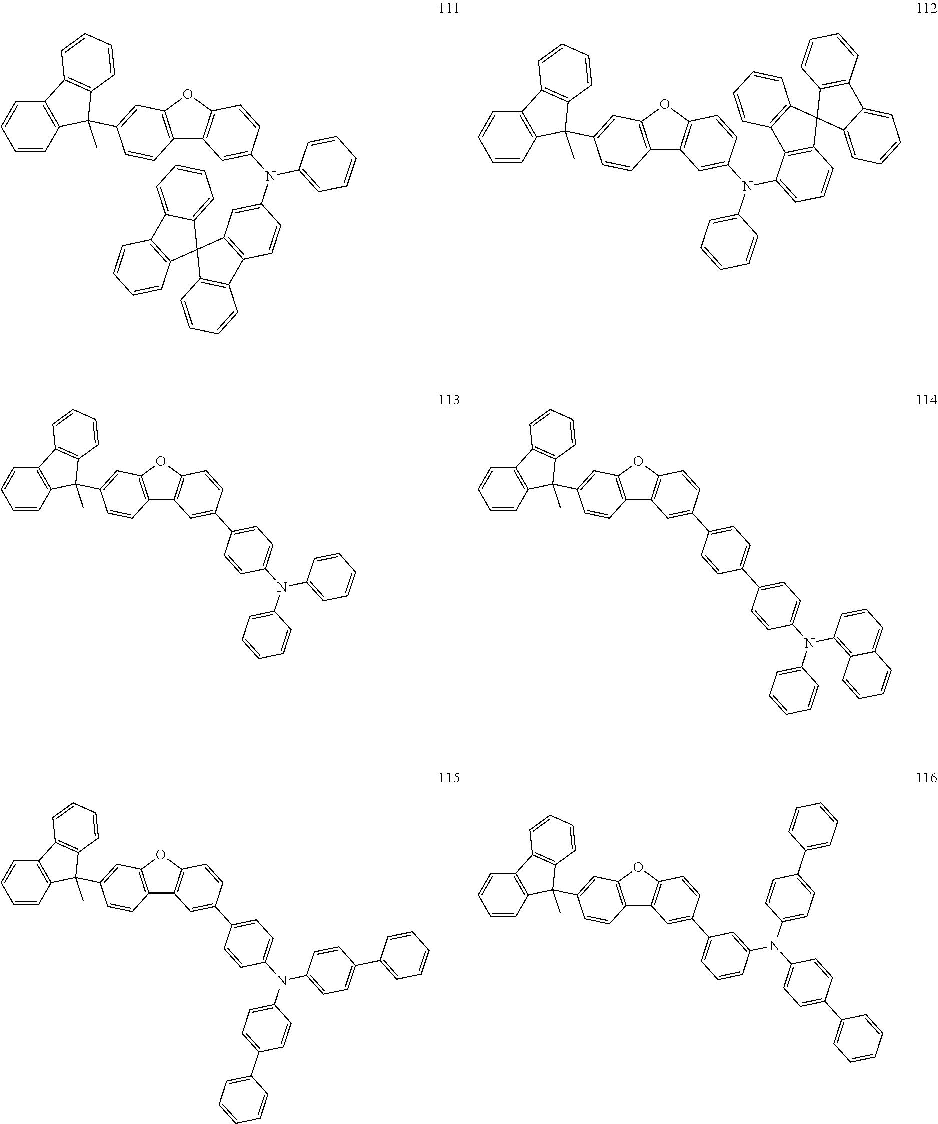

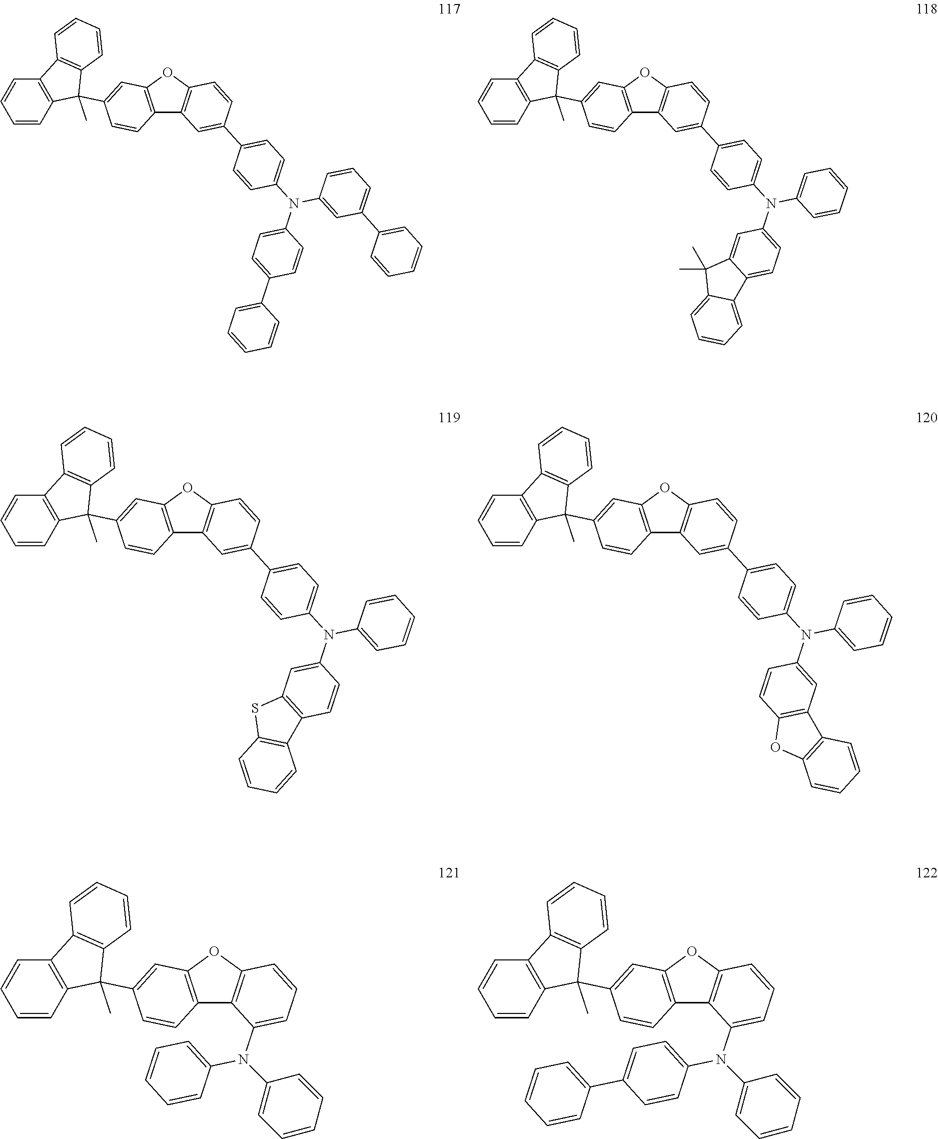

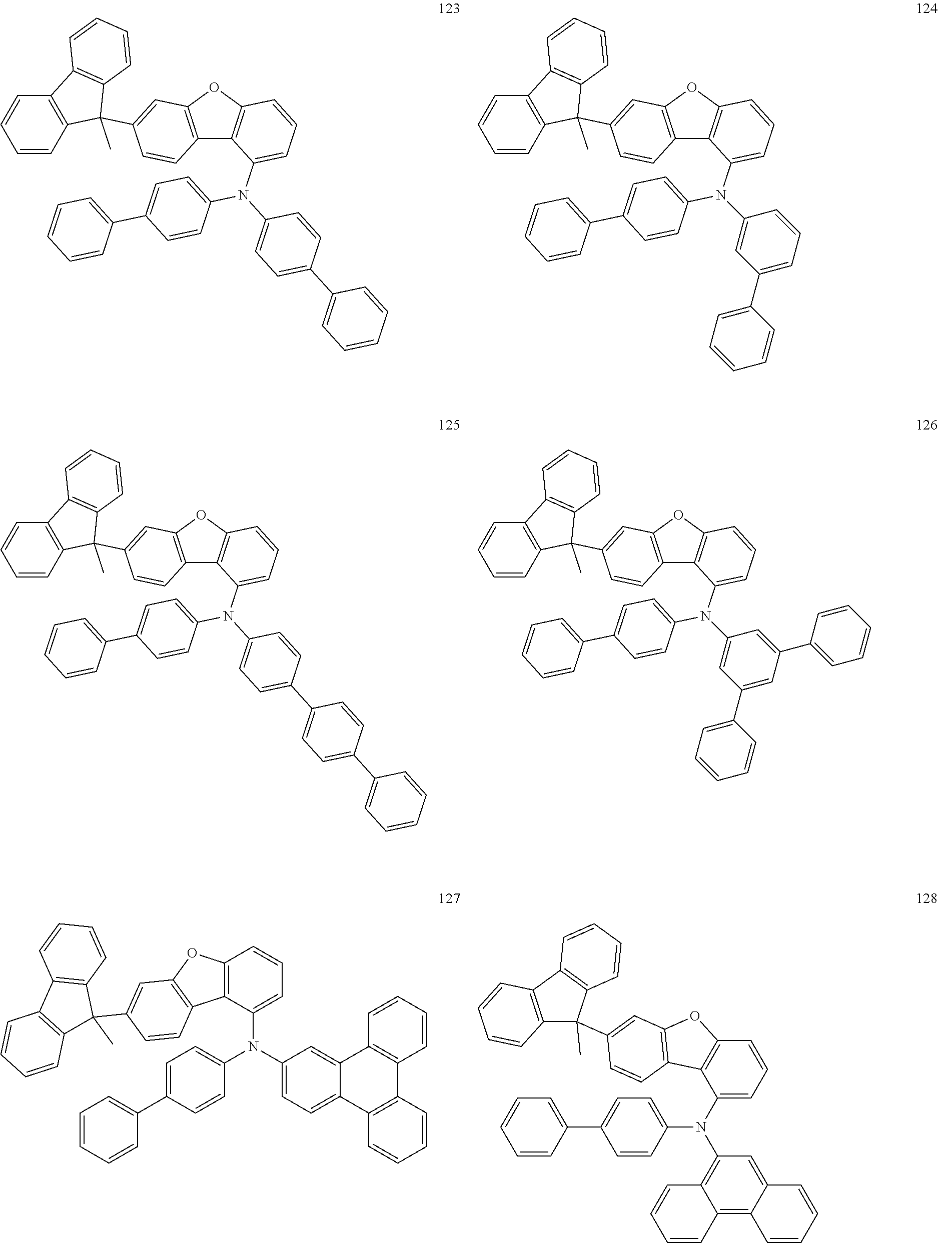

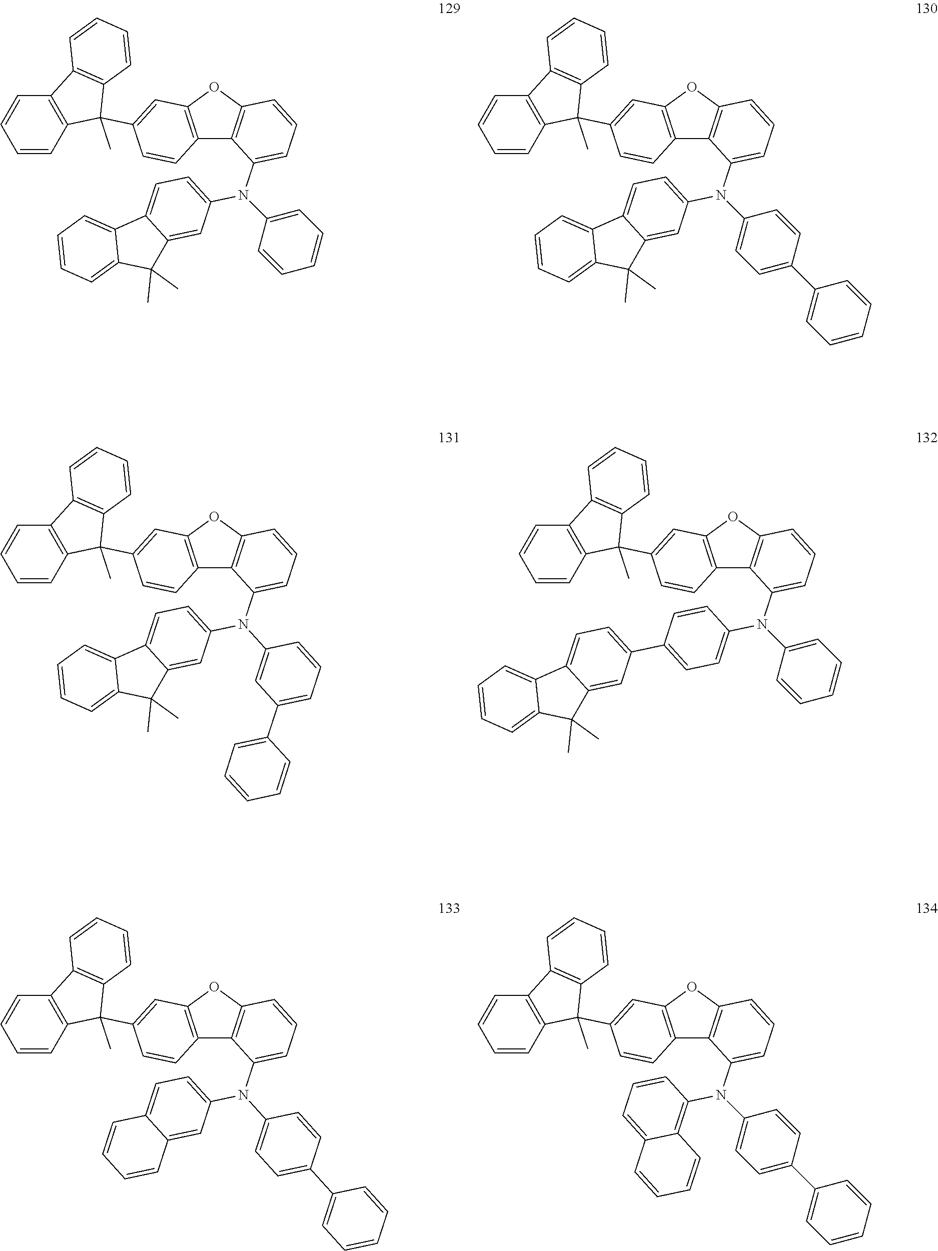

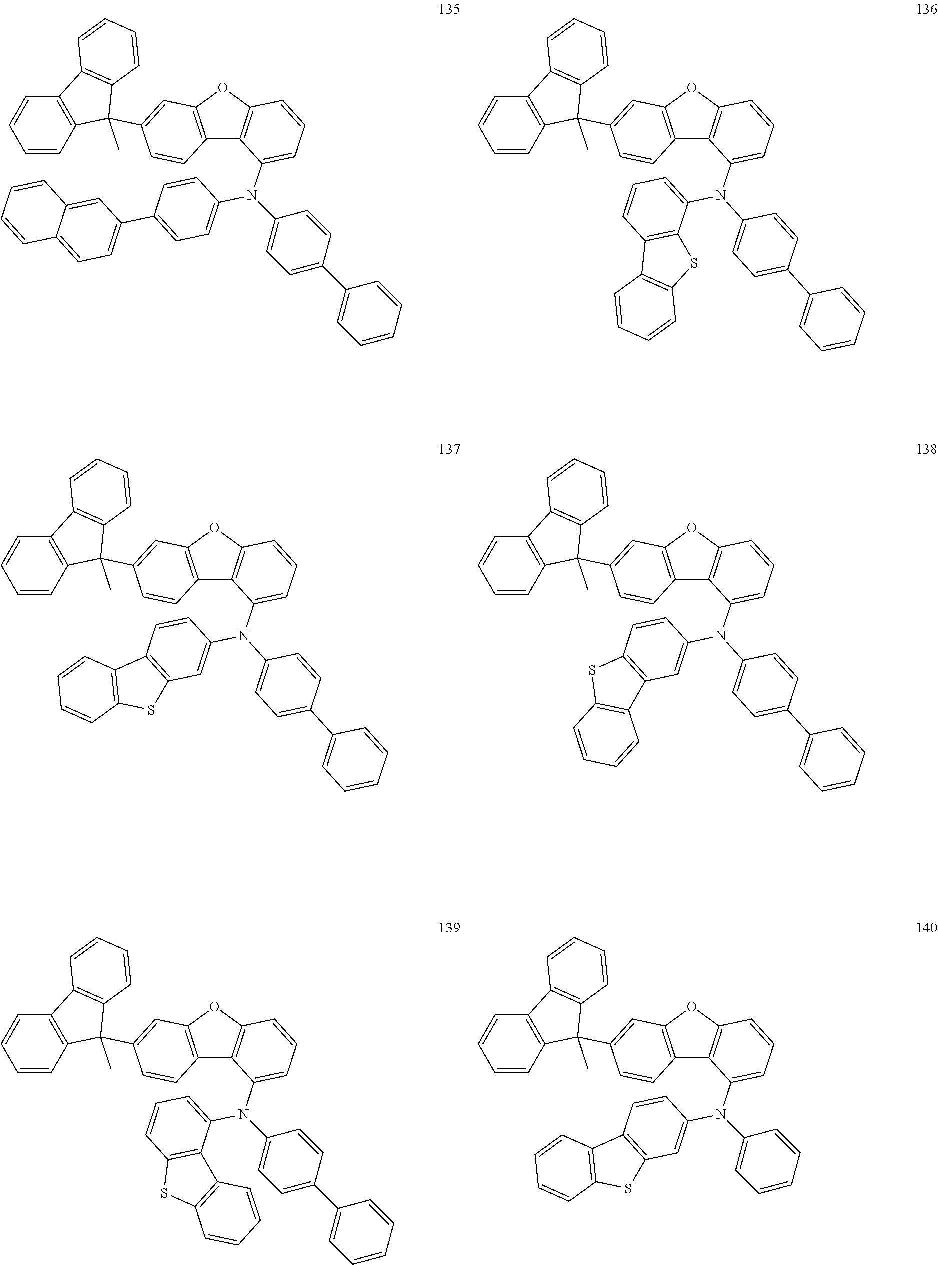

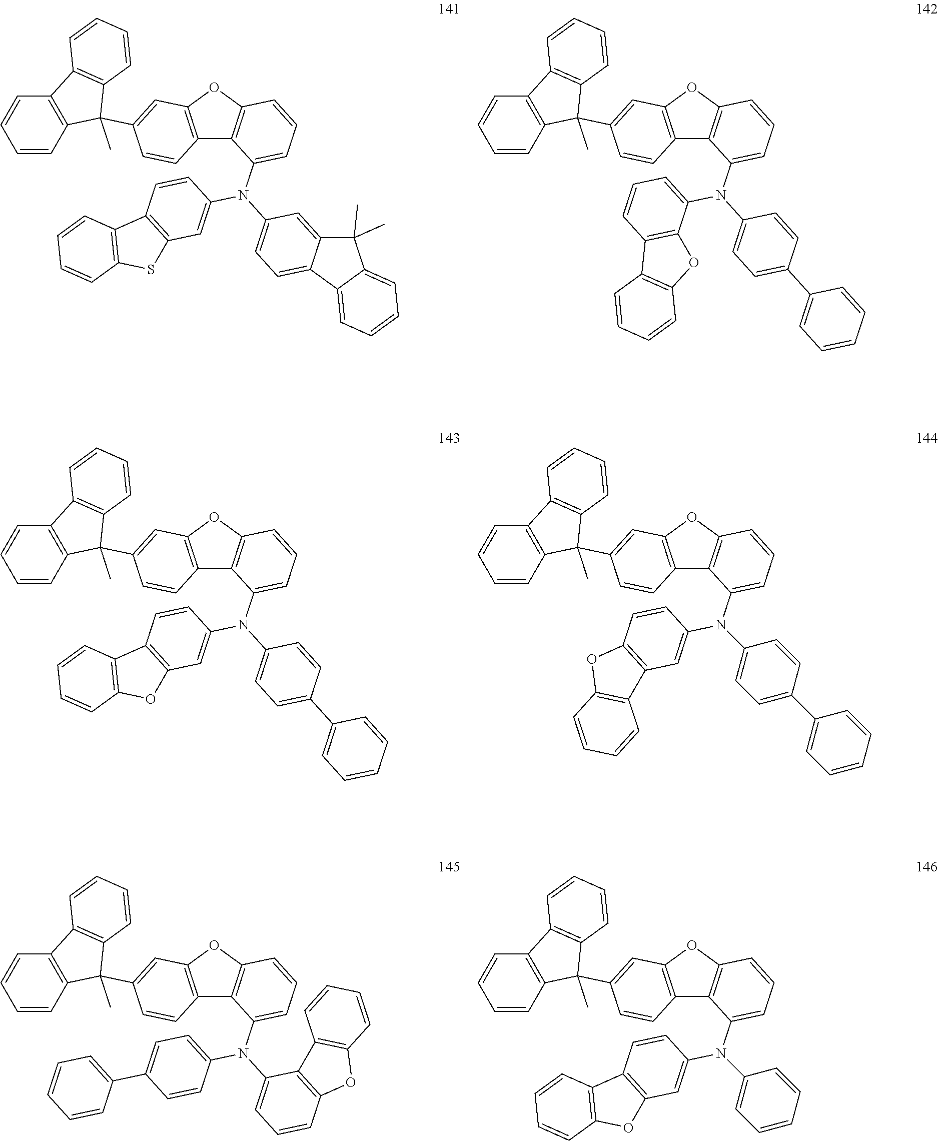

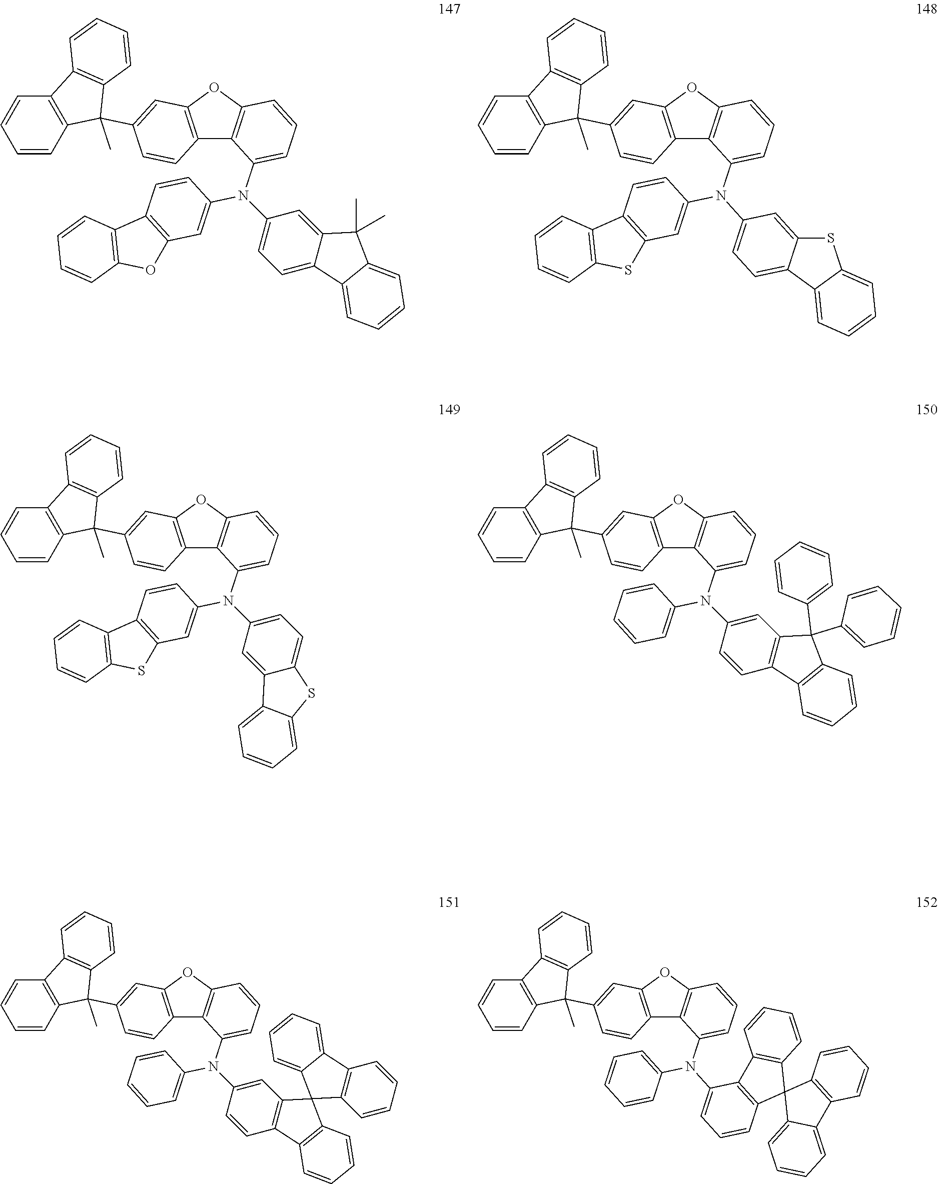

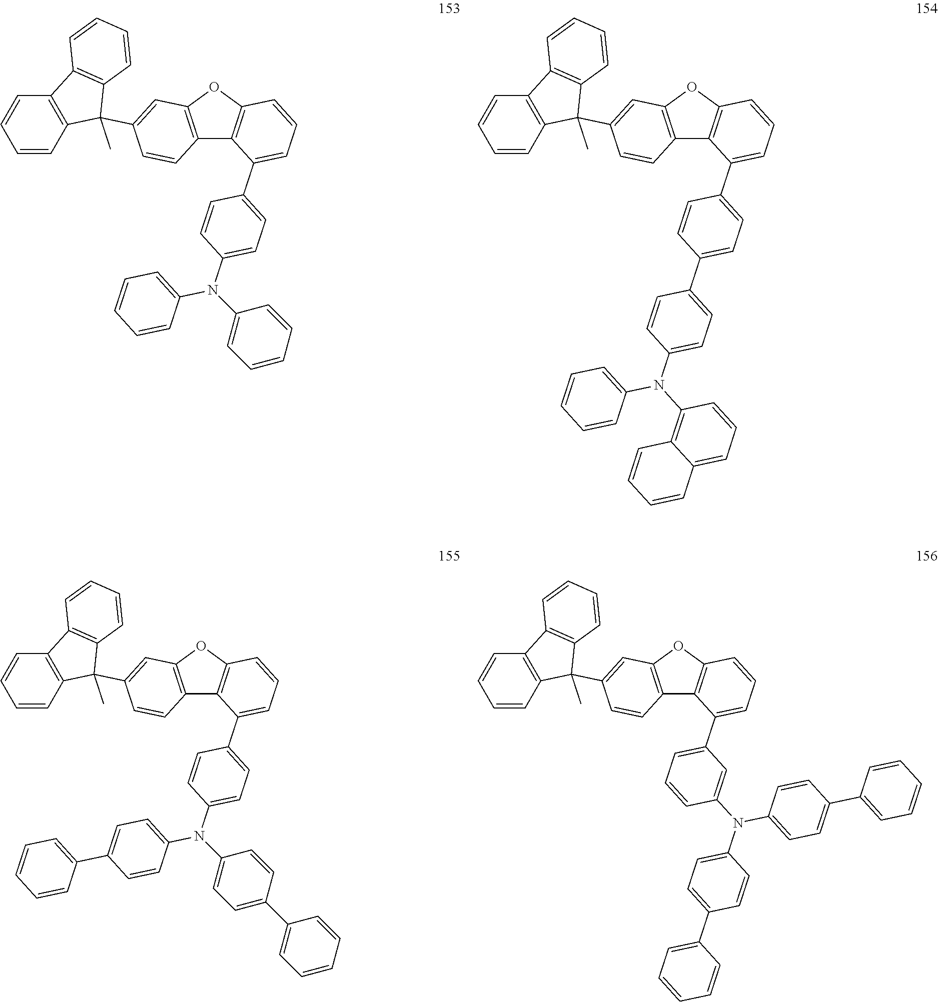 [SMILES: CC1(c2ccc3c(c2)oc2cccc(-c4ccc(N(c5ccccc5)c5ccccc5)cc4)c23)c2ccccc2-c2ccccc21 ; CC1(c2ccc3c(c2)oc2cccc(-c4ccc(-c5ccc(N(c6ccccc6)c6cccc7ccccc67)cc5)cc4)c23)c2ccccc2-c2ccccc21 ; CC1(c2ccc3c(c2)oc2cccc(-c4ccc(N(c5ccc(-c6ccccc6)cc5)c5ccc(-c6ccccc6)cc5)cc4)c23)c2ccccc2-c2ccccc21 ; CC1(c2ccc3c(c2)oc2cccc(-c4cccc(N(c5ccc(-c6ccccc6)cc5)c5ccc(-c6ccccc6)cc5)c4)c23)c2ccccc2-c2ccccc21]

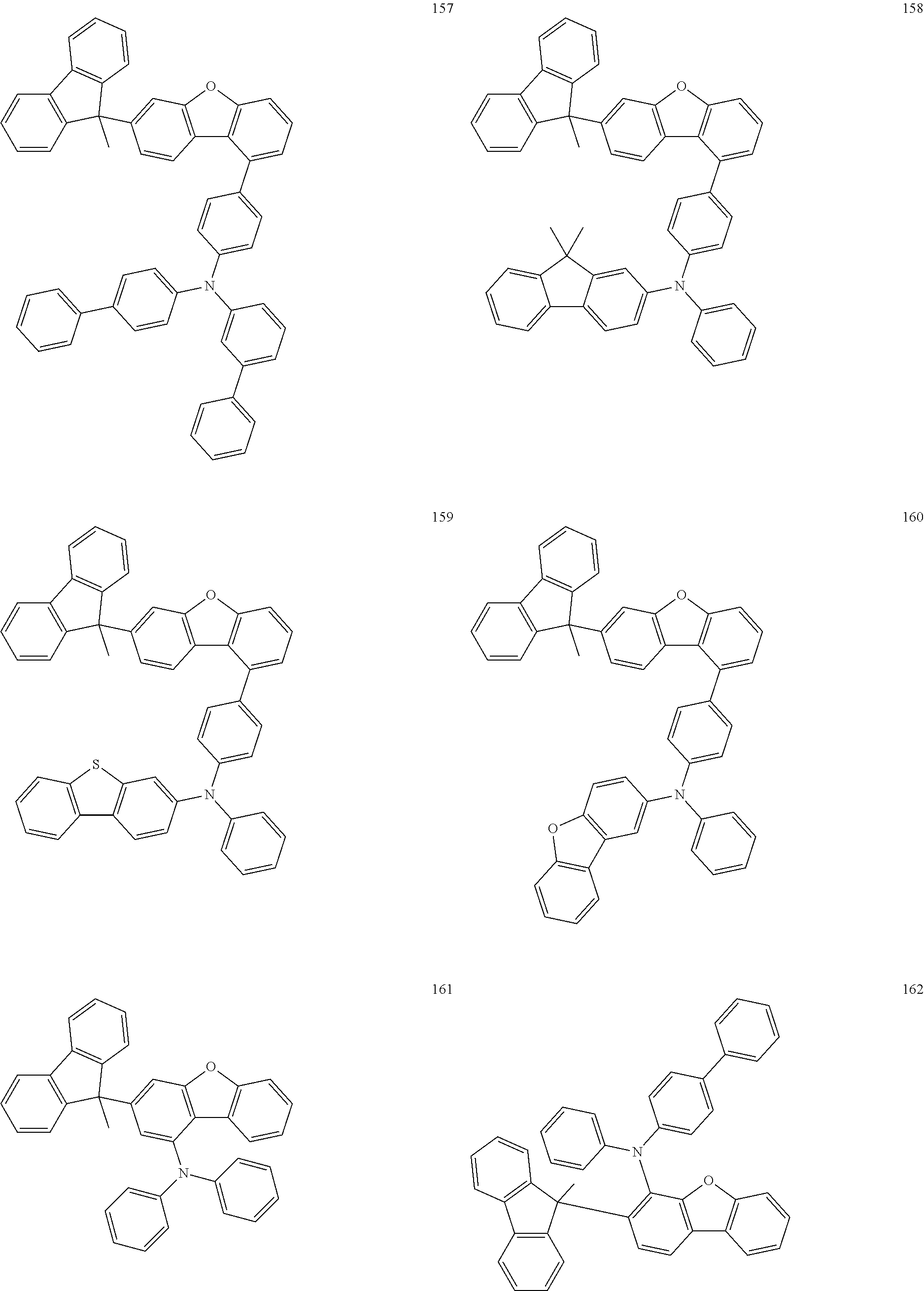

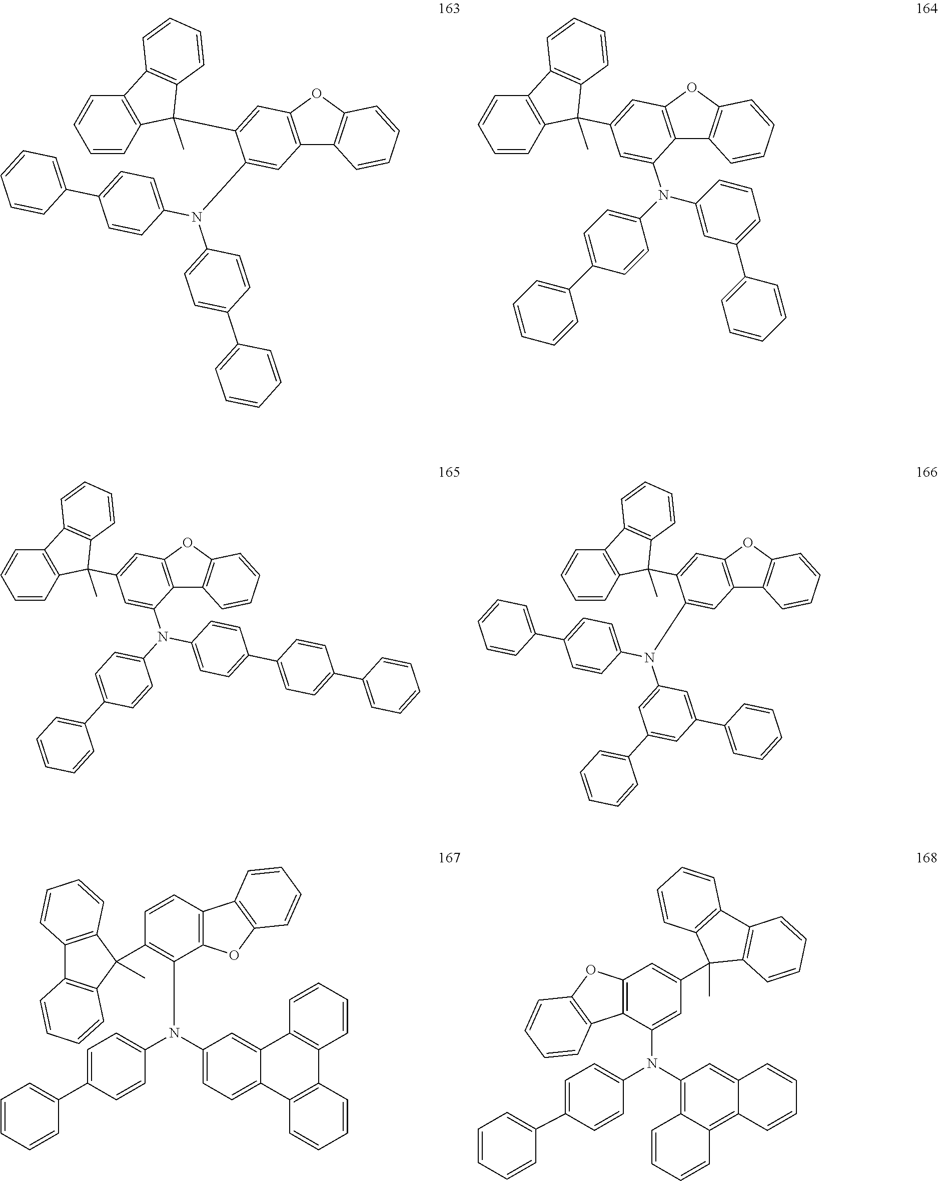

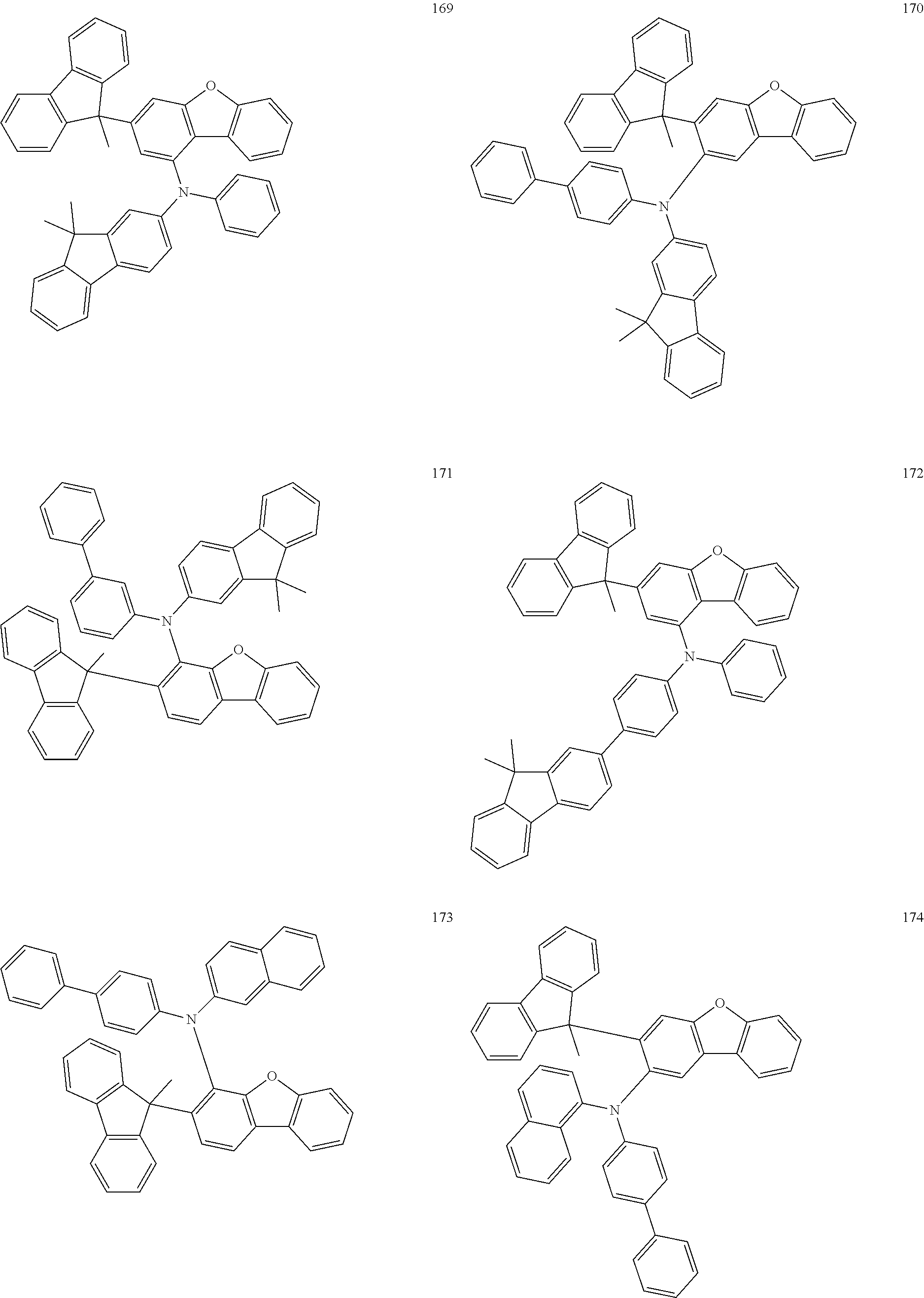

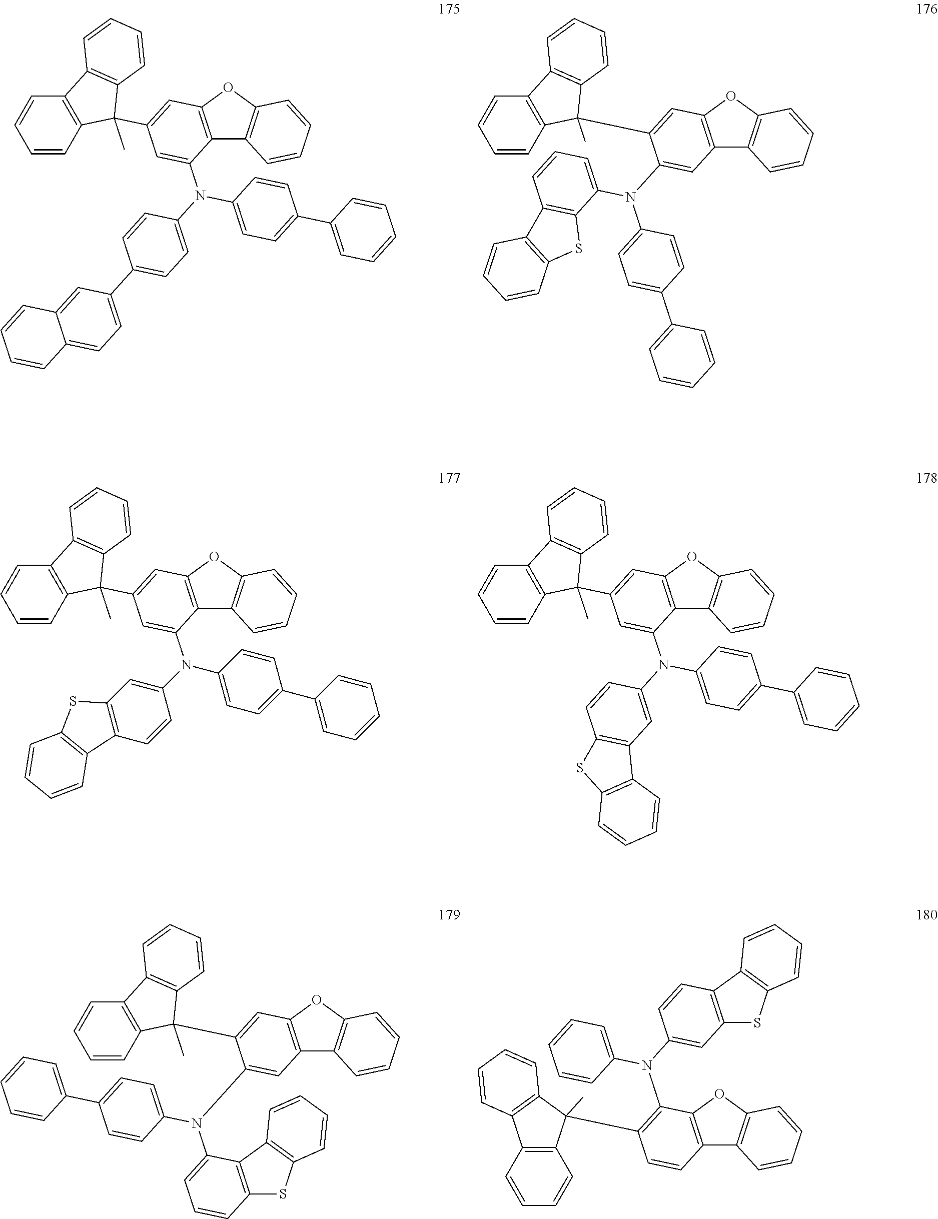

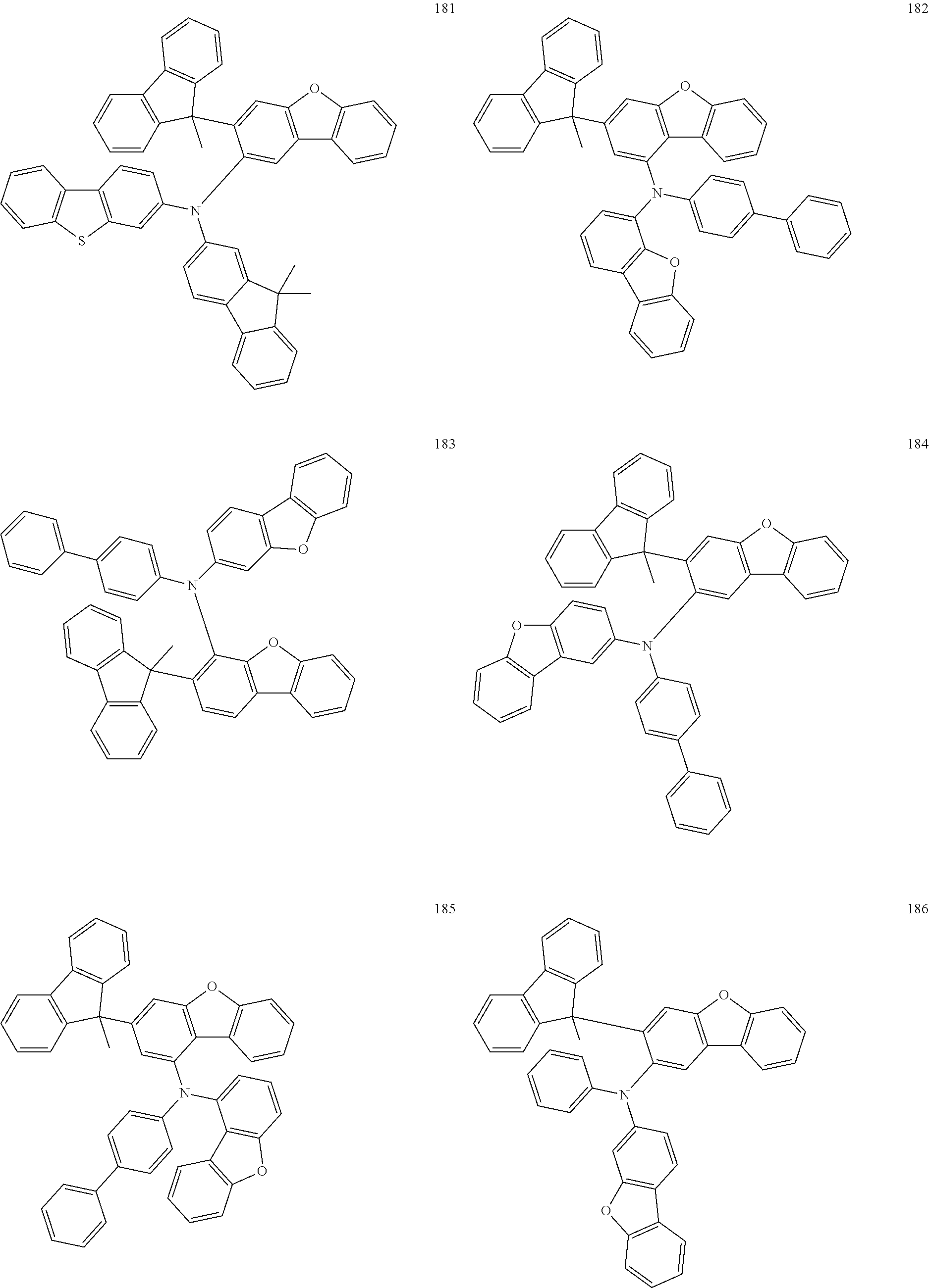

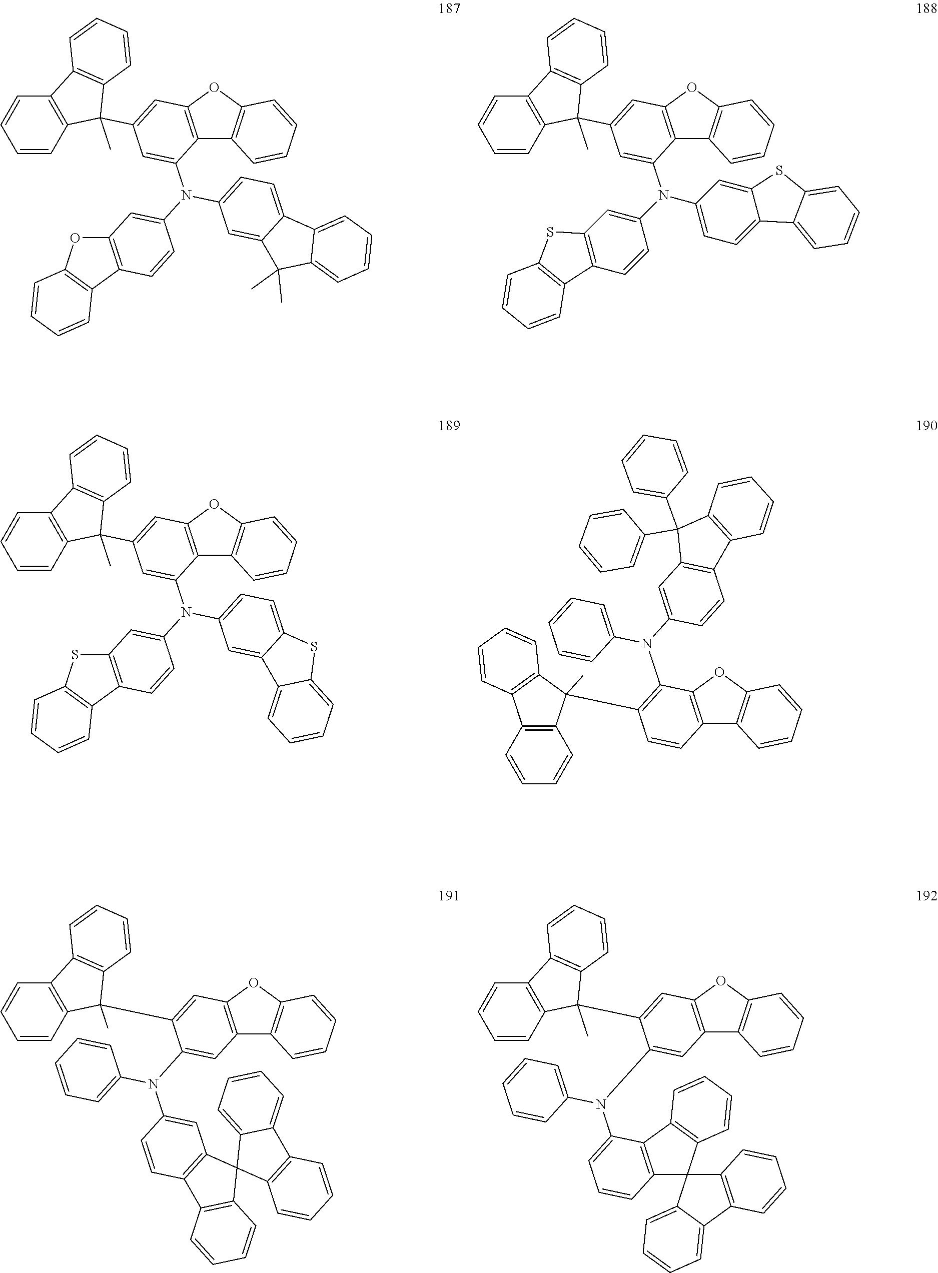

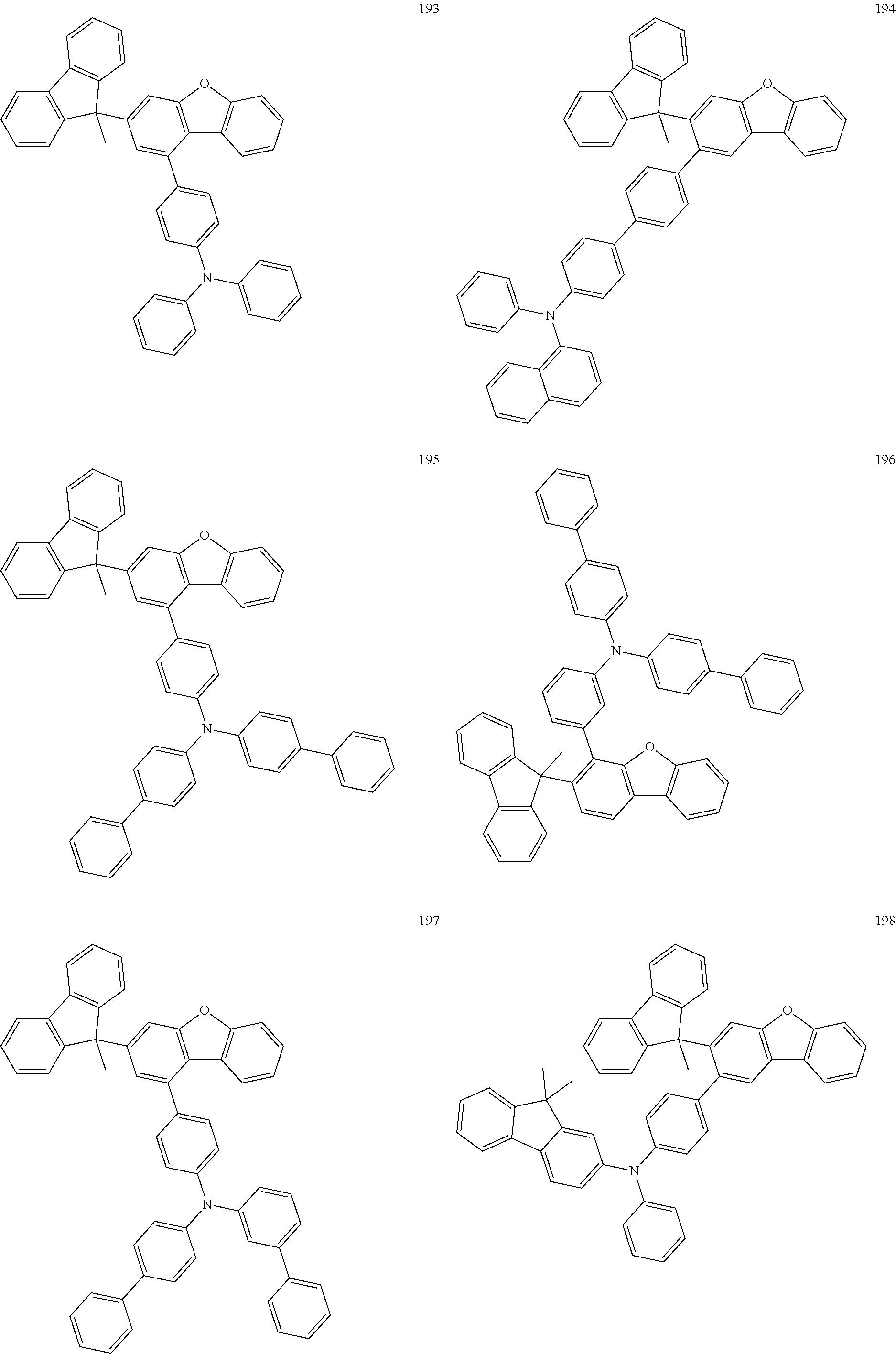

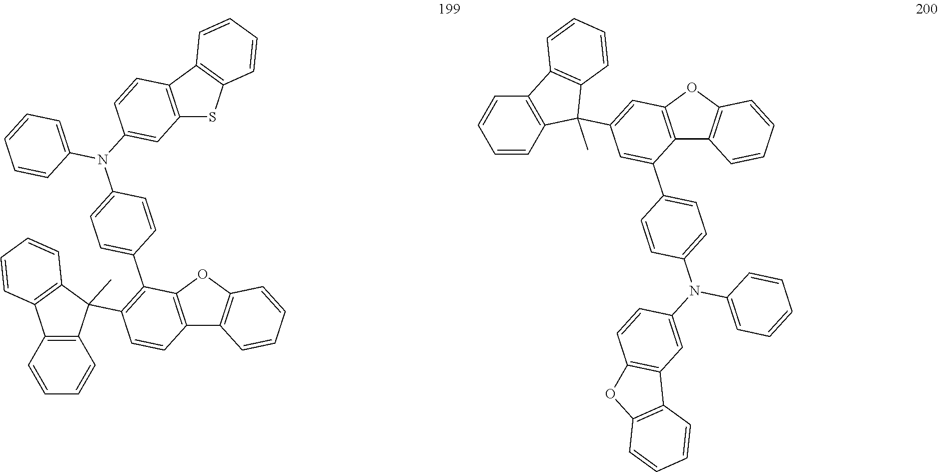 [SMILES: CC1(c2ccc3c(oc4ccccc43)c2-c2ccc(N(c3ccccc3)c3ccc4c(c3)sc3ccccc34)cc2)c2ccccc2-c2ccccc21 ; CC1(c2cc(-c3ccc(N(c4ccccc4)c4ccc5oc6ccccc6c5c4)cc3)c3c(c2)oc2ccccc23)c2ccccc2-c2ccccc21]

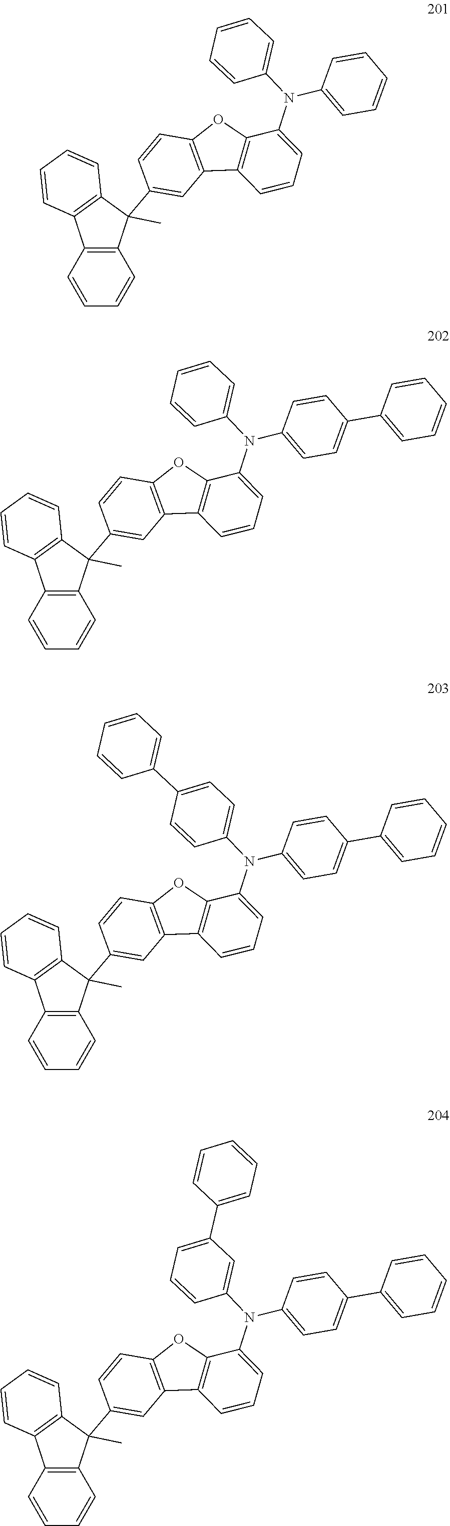 [SMILES: CC1(c2ccc3oc4c(N(c5ccccc5)c5ccccc5)cccc4c3c2)c2ccccc2-c2ccccc21 ; CC1(c2ccc3oc4c(N(c5ccccc5)c5ccc(-c6ccccc6)cc5)cccc4c3c2)c2ccccc2-c2ccccc21 ; CC1(c2ccc3oc4c(N(c5ccc(-c6ccccc6)cc5)c5ccc(-c6ccccc6)cc5)cccc4c3c2)c2ccccc2-c2ccccc21 ; CC1(c2ccc3oc4c(N(c5ccc(-c6ccccc6)cc5)c5cccc(-c6ccccc6)c5)cccc4c3c2)c2ccccc2-c2ccccc21]

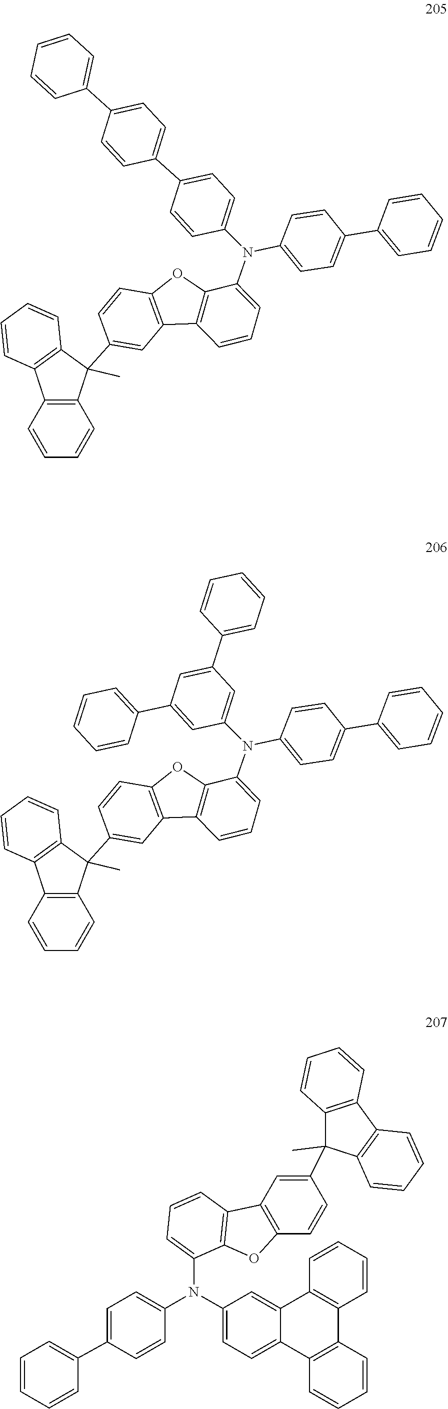 [SMILES: CC1(c2ccc3oc4c(N(c5ccc(-c6ccccc6)cc5)c5ccc(-c6ccc(-c7ccccc7)cc6)cc5)cccc4c3c2)c2ccccc2-c2ccccc21 ; CC1(c2ccc3oc4c(N(c5ccc(-c6ccccc6)cc5)c5cc(-c6ccccc6)cc(-c6ccccc6)c5)cccc4c3c2)c2ccccc2-c2ccccc21 ; CC1(c2ccc3oc4c(N(c5ccc(-c6ccccc6)cc5)c5ccc6c7ccccc7c7ccccc7c6c5)cccc4c3c2)c2ccccc2-c2ccccc21]

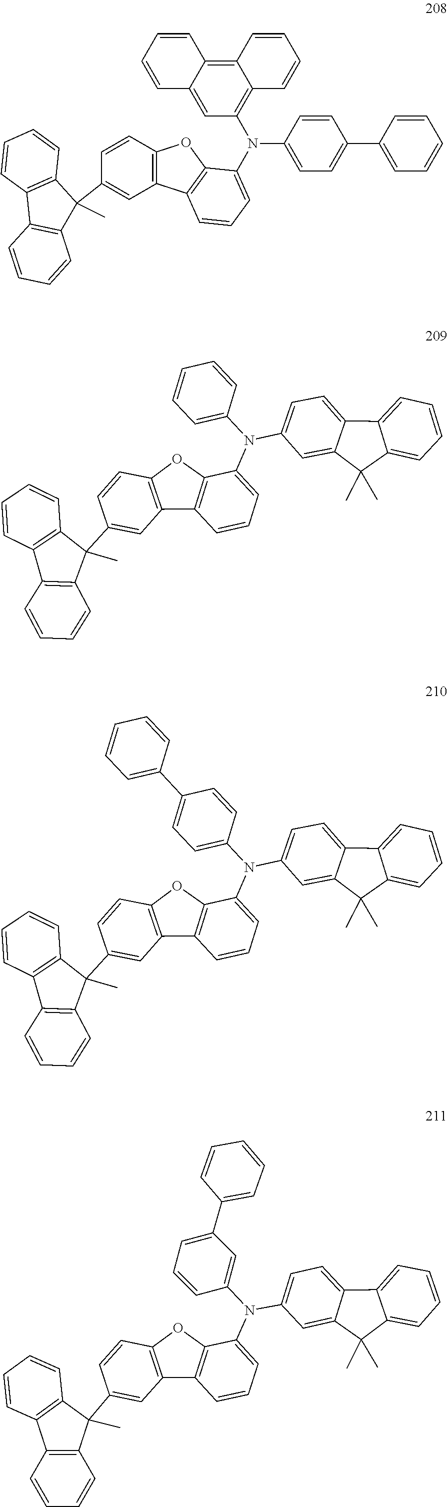 [SMILES: CC1(c2ccc3oc4c(N(c5ccc(-c6ccccc6)cc5)c5cc6ccccc6c6ccccc56)cccc4c3c2)c2ccccc2-c2ccccc21 ; CC1(C)c2ccccc2-c2ccc(N(c3ccccc3)c3cccc4c3oc3ccc(C5(C)c6ccccc6-c6ccccc65)cc34)cc21 ; CC1(C)c2ccccc2-c2ccc(N(c3ccc(-c4ccccc4)cc3)c3cccc4c3oc3ccc(C5(C)c6ccccc6-c6ccccc65)cc34)cc21 ; CC1(C)c2ccccc2-c2ccc(N(c3cccc(-c4ccccc4)c3)c3cccc4c3oc3ccc(C5(C)c6ccccc6-c6ccccc65)cc34)cc21]

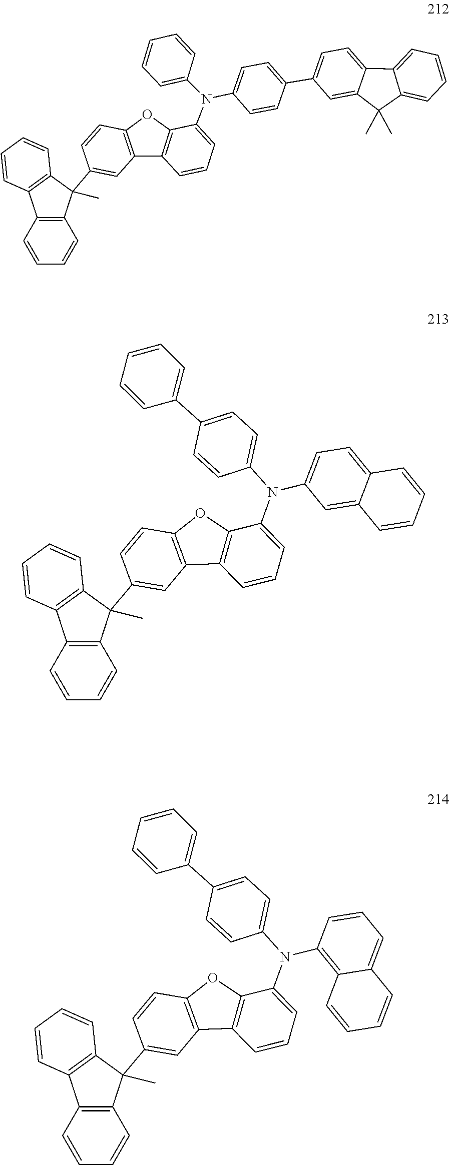 [SMILES: CC1(C)c2ccccc2-c2ccc(-c3ccc(N(c4ccccc4)c4cccc5c4oc4ccc(C6(C)c7ccccc7-c7ccccc76)cc45)cc3)cc21 ; CC1(c2ccc3oc4c(N(c5ccc(-c6ccccc6)cc5)c5ccc6ccccc6c5)cccc4c3c2)c2ccccc2-c2ccccc21 ; CC1(c2ccc3oc4c(N(c5ccc(-c6ccccc6)cc5)c5cccc6ccccc56)cccc4c3c2)c2ccccc2-c2ccccc21]

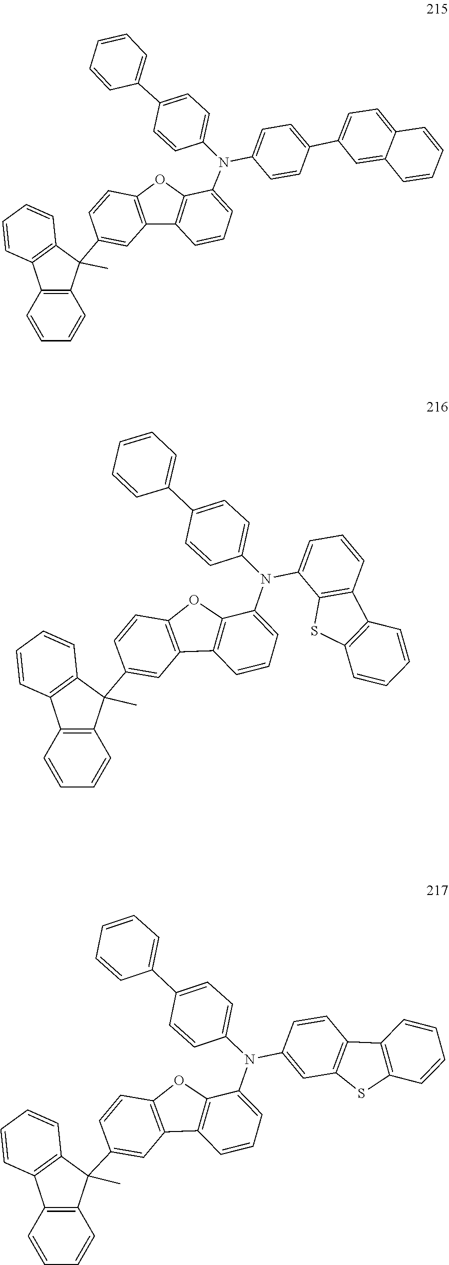 [SMILES: CC1(c2ccc3oc4c(N(c5ccc(-c6ccccc6)cc5)c5ccc(-c6ccc7ccccc7c6)cc5)cccc4c3c2)c2ccccc2-c2ccccc21 ; CC1(c2ccc3oc4c(N(c5ccc(-c6ccccc6)cc5)c5cccc6c5sc5ccccc56)cccc4c3c2)c2ccccc2-c2ccccc21 ; CC1(c2ccc3oc4c(N(c5ccc(-c6ccccc6)cc5)c5ccc6c(c5)sc5ccccc56)cccc4c3c2)c2ccccc2-c2ccccc21]

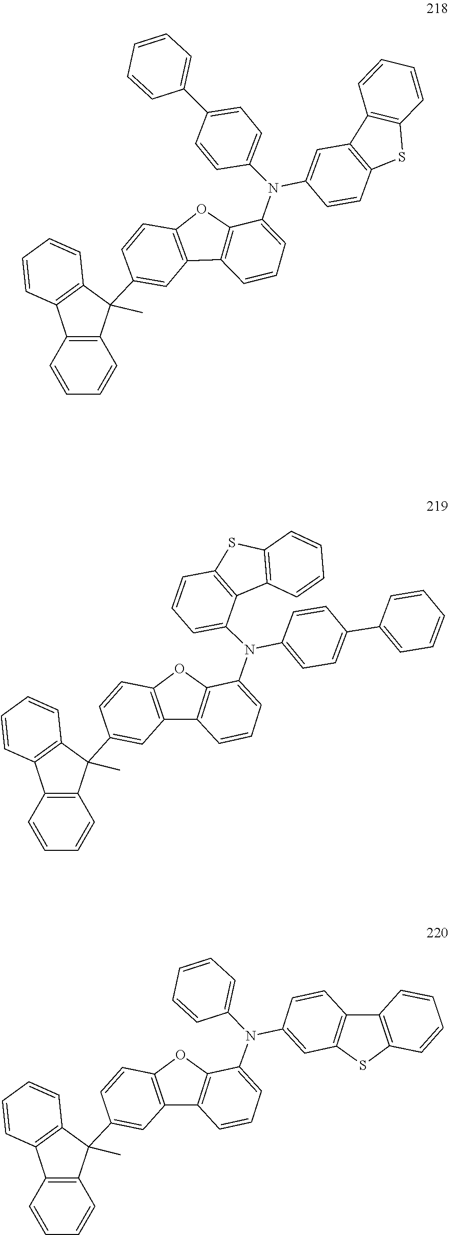 [SMILES: CC1(c2ccc3oc4c(N(c5ccc(-c6ccccc6)cc5)c5ccc6sc7ccccc7c6c5)cccc4c3c2)c2ccccc2-c2ccccc21 ; CC1(c2ccc3oc4c(N(c5ccc(-c6ccccc6)cc5)c5cccc6sc7ccccc7c56)cccc4c3c2)c2ccccc2-c2ccccc21 ; CC1(c2ccc3oc4c(N(c5ccccc5)c5ccc6c(c5)sc5ccccc56)cccc4c3c2)c2ccccc2-c2ccccc21]

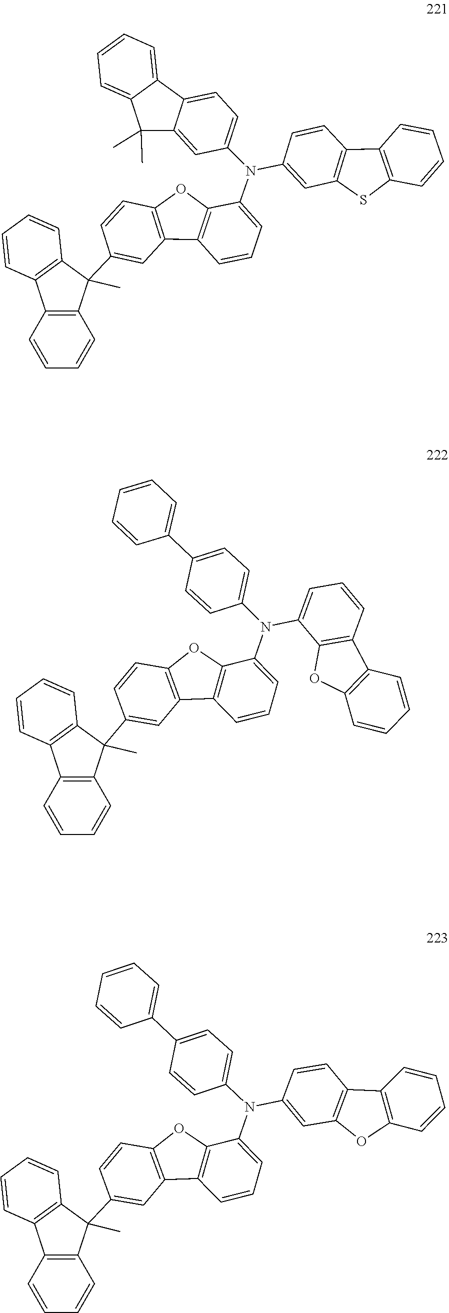 [SMILES: CC1(C)c2ccccc2-c2ccc(N(c3ccc4c(c3)sc3ccccc34)c3cccc4c3oc3ccc(C5(C)c6ccccc6-c6ccccc65)cc34)cc21 ; CC1(c2ccc3oc4c(N(c5ccc(-c6ccccc6)cc5)c5cccc6c5oc5ccccc56)cccc4c3c2)c2ccccc2-c2ccccc21 ; CC1(c2ccc3oc4c(N(c5ccc(-c6ccccc6)cc5)c5ccc6c(c5)oc5ccccc56)cccc4c3c2)c2ccccc2-c2ccccc21]

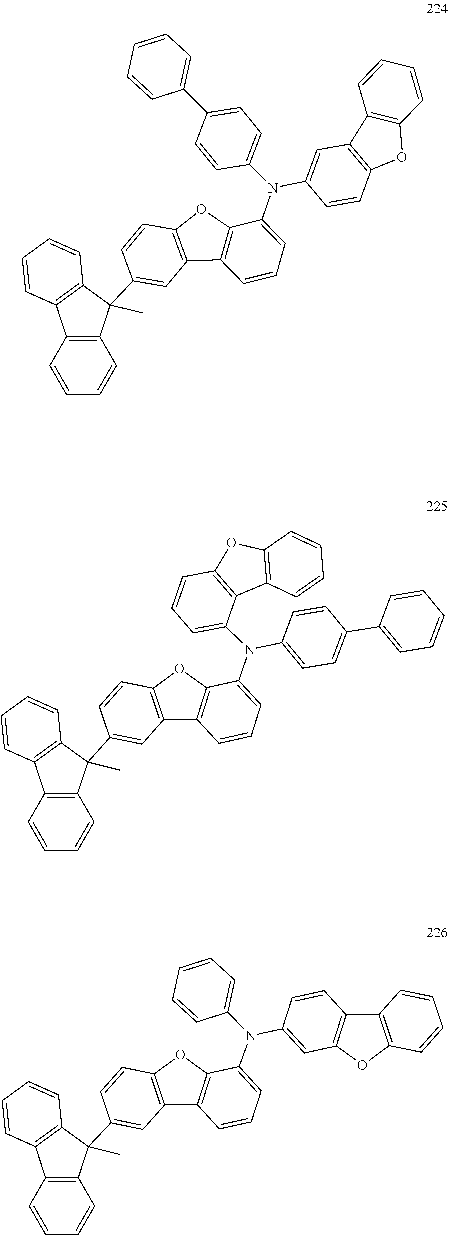 [SMILES: CC1(c2ccc3oc4c(N(c5ccc(-c6ccccc6)cc5)c5ccc6oc7ccccc7c6c5)cccc4c3c2)c2ccccc2-c2ccccc21 ; CC1(c2ccc3oc4c(N(c5ccc(-c6ccccc6)cc5)c5cccc6oc7ccccc7c56)cccc4c3c2)c2ccccc2-c2ccccc21 ; CC1(c2ccc3oc4c(N(c5ccccc5)c5ccc6c(c5)oc5ccccc56)cccc4c3c2)c2ccccc2-c2ccccc21]

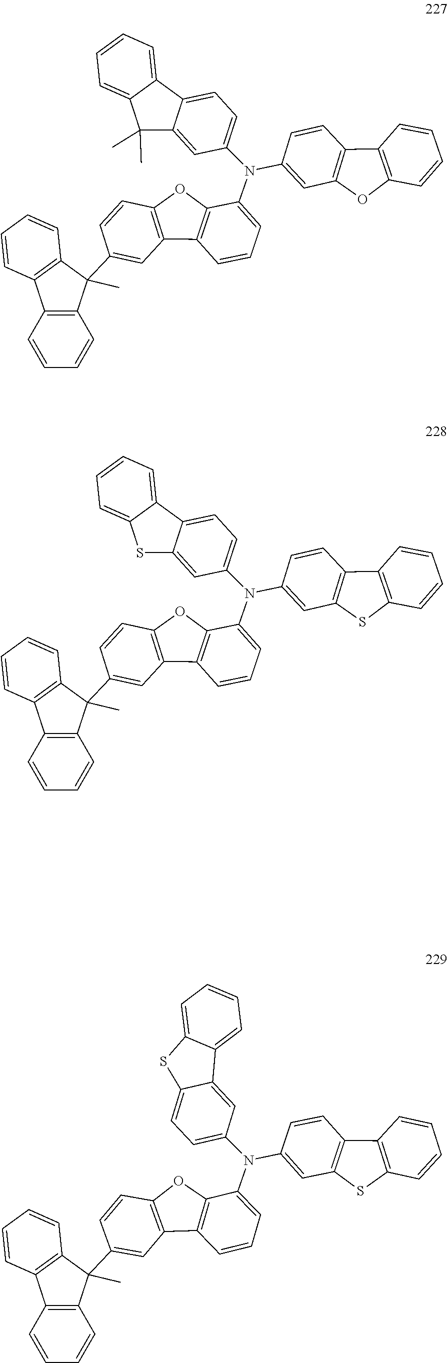 [SMILES: CC1(C)c2ccccc2-c2ccc(N(c3ccc4c(c3)oc3ccccc34)c3cccc4c3oc3ccc(C5(C)c6ccccc6-c6ccccc65)cc34)cc21 ; CC1(c2ccc3oc4c(N(c5ccc6c(c5)sc5ccccc56)c5ccc6c(c5)sc5ccccc56)cccc4c3c2)c2ccccc2-c2ccccc21 ; CC1(c2ccc3oc4c(N(c5ccc6c(c5)sc5ccccc56)c5ccc6sc7ccccc7c6c5)cccc4c3c2)c2ccccc2-c2ccccc21]

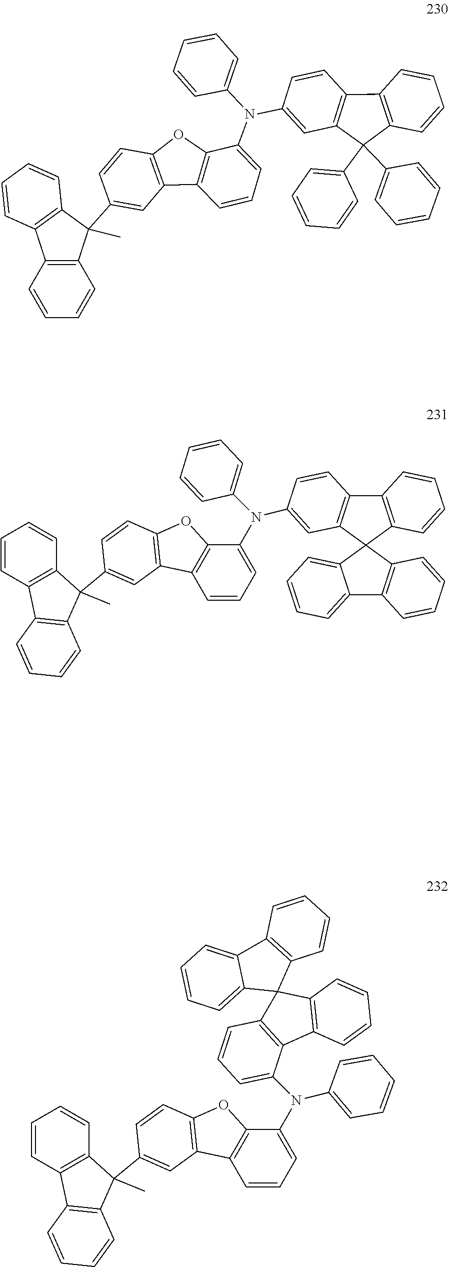 [SMILES: CC1(c2ccc3oc4c(N(c5ccccc5)c5ccc6c(c5)C(c5ccccc5)(c5ccccc5)c5ccccc5-6)cccc4c3c2)c2ccccc2-c2ccccc21 ; CC1(c2ccc3oc4c(N(c5ccccc5)c5ccc6c(c5)C5(c7ccccc7-c7ccccc75)c5ccccc5-6)cccc4c3c2)c2ccccc2-c2ccccc21 ; CC1(c2ccc3oc4c(N(c5ccccc5)c5cccc6c5-c5ccccc5C65c6ccccc6-c6ccccc65)cccc4c3c2)c2ccccc2-c2ccccc21]

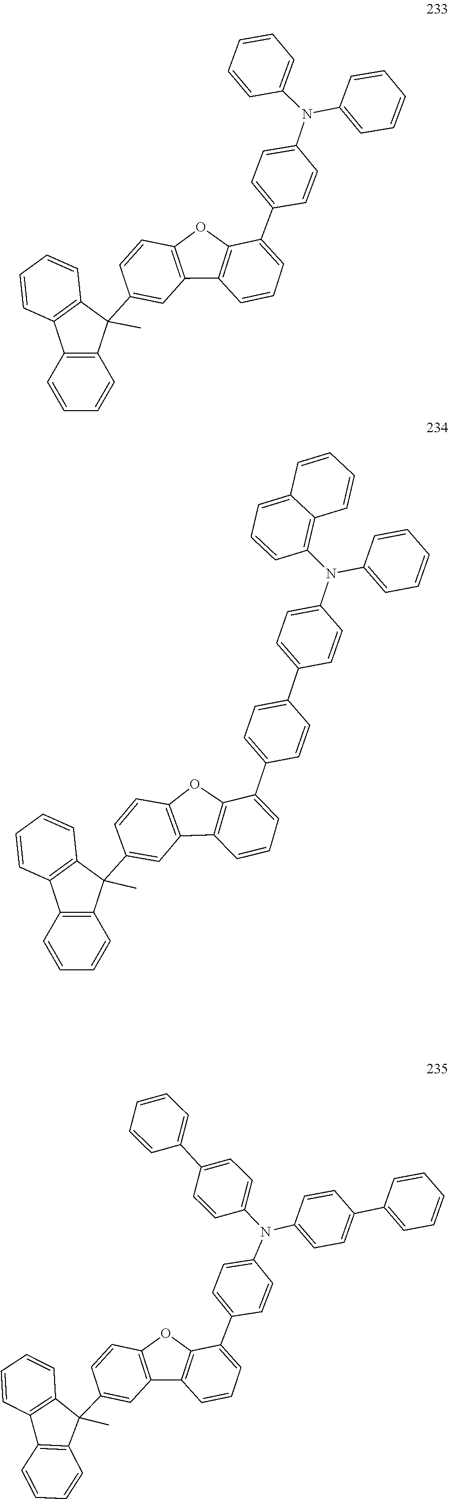 [SMILES: CC1(c2ccc3oc4c(-c5ccc(N(c6ccccc6)c6ccccc6)cc5)cccc4c3c2)c2ccccc2-c2ccccc21 ; CC1(c2ccc3oc4c(-c5ccc(-c6ccc(N(c7ccccc7)c7cccc8ccccc78)cc6)cc5)cccc4c3c2)c2ccccc2-c2ccccc21 ; CC1(c2ccc3oc4c(-c5ccc(N(c6ccc(-c7ccccc7)cc6)c6ccc(-c7ccccc7)cc6)cc5)cccc4c3c2)c2ccccc2-c2ccccc21]

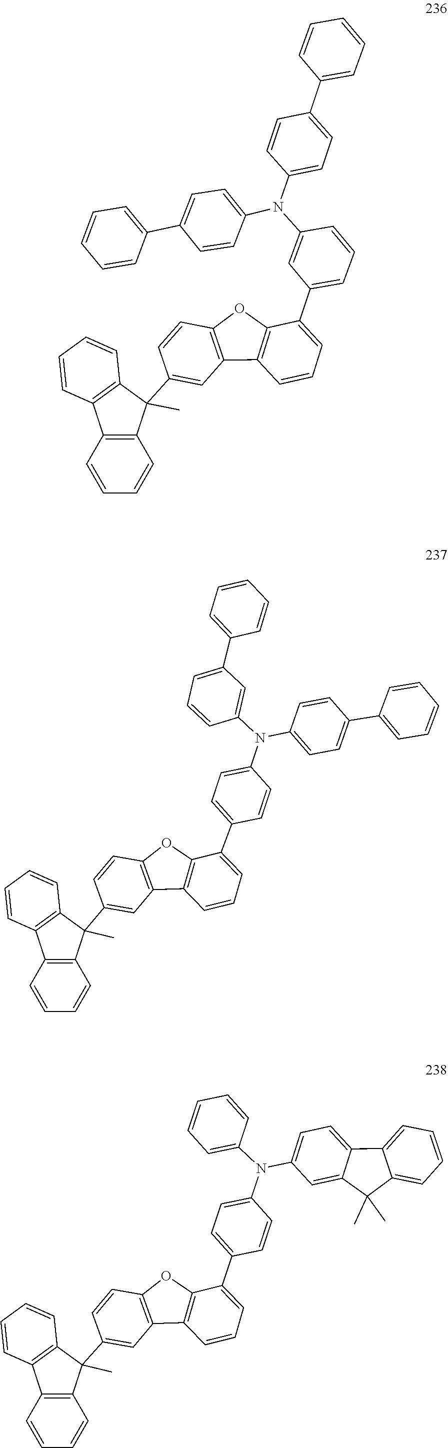 [SMILES: CC1(c2ccc3oc4c(-c5cccc(N(c6ccc(-c7ccccc7)cc6)c6ccc(-c7ccccc7)cc6)c5)cccc4c3c2)c2ccccc2-c2ccccc21 ; CC1(c2ccc3oc4c(-c5ccc(N(c6ccc(-c7ccccc7)cc6)c6cccc(-c7ccccc7)c6)cc5)cccc4c3c2)c2ccccc2-c2ccccc21 ; CC1(C)c2ccccc2-c2ccc(N(c3ccccc3)c3ccc(-c4cccc5c4oc4ccc(C6(C)c7ccccc7-c7ccccc76)cc45)cc3)cc21]

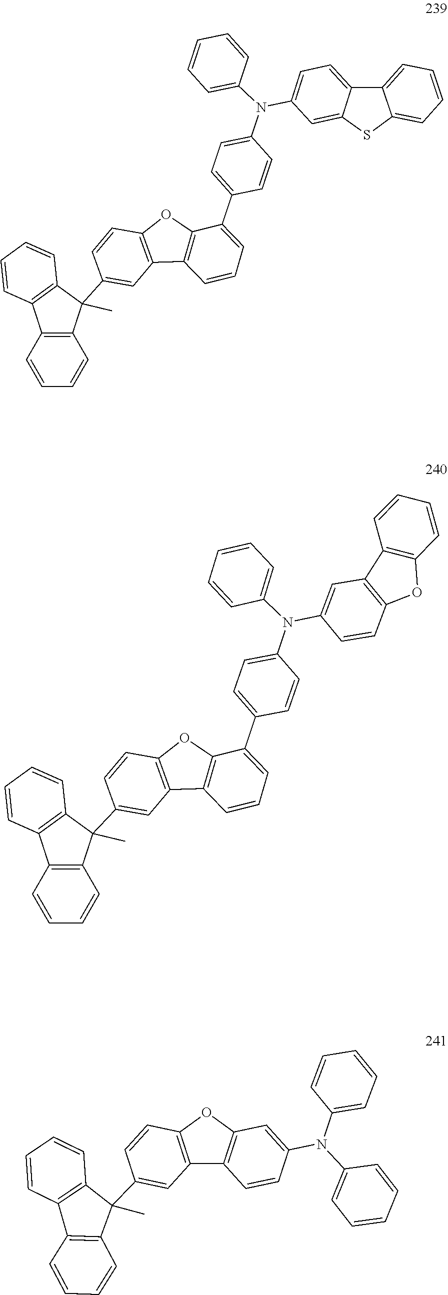 [SMILES: CC1(c2ccc3oc4c(-c5ccc(N(c6ccccc6)c6ccc7c(c6)sc6ccccc67)cc5)cccc4c3c2)c2ccccc2-c2ccccc21 ; CC1(c2ccc3oc4c(-c5ccc(N(c6ccccc6)c6ccc7oc8ccccc8c7c6)cc5)cccc4c3c2)c2ccccc2-c2ccccc21 ; CC1(c2ccc3oc4cc(N(c5ccccc5)c5ccccc5)ccc4c3c2)c2ccccc2-c2ccccc21]

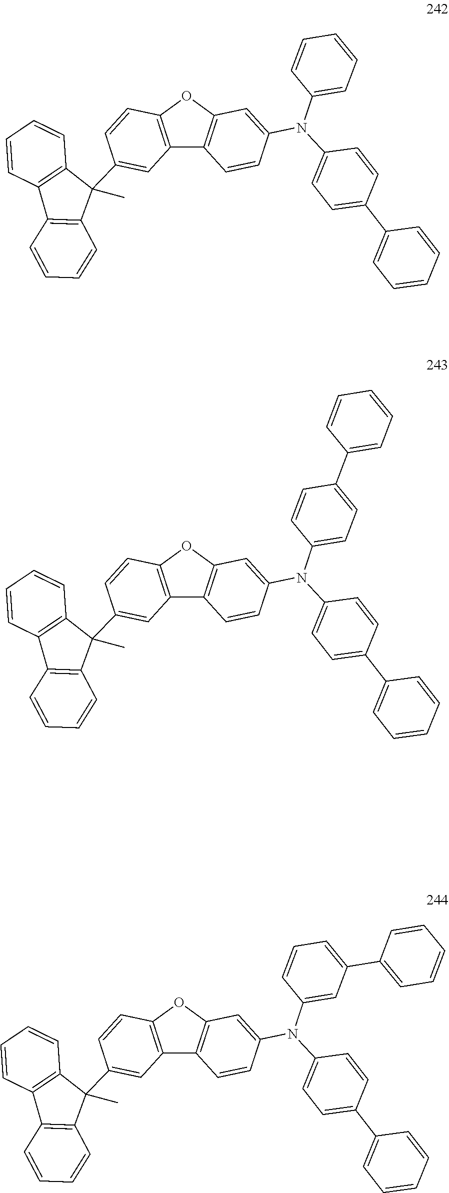 [SMILES: CC1(c2ccc3oc4cc(N(c5ccccc5)c5ccc(-c6ccccc6)cc5)ccc4c3c2)c2ccccc2-c2ccccc21 ; CC1(c2ccc3oc4cc(N(c5ccc(-c6ccccc6)cc5)c5ccc(-c6ccccc6)cc5)ccc4c3c2)c2ccccc2-c2ccccc21 ; CC1(c2ccc3oc4cc(N(c5ccc(-c6ccccc6)cc5)c5cccc(-c6ccccc6)c5)ccc4c3c2)c2ccccc2-c2ccccc21]

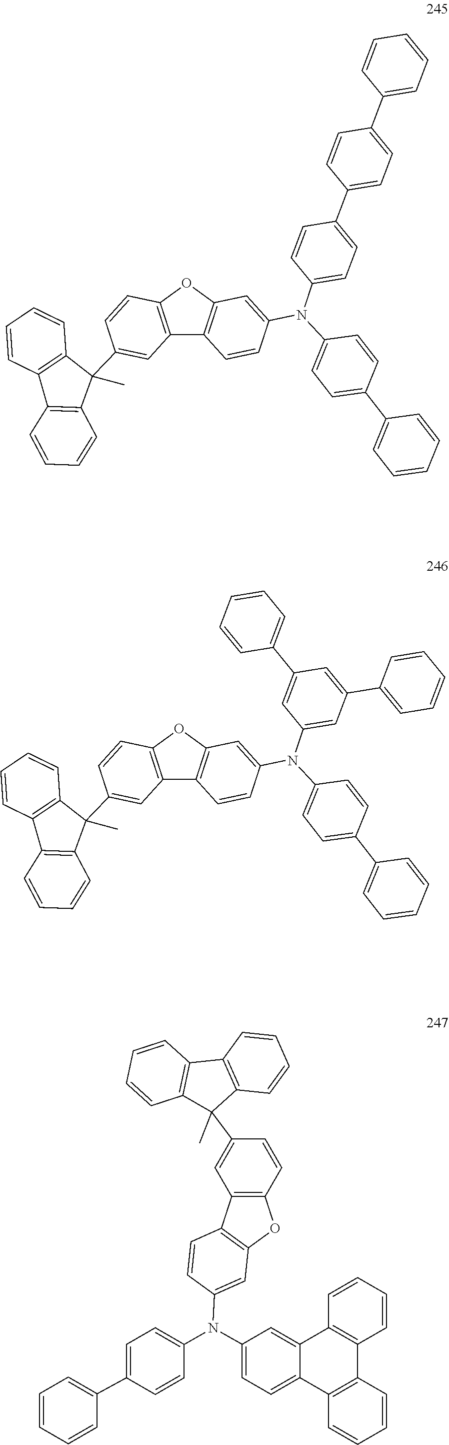 [SMILES: CC1(c2ccc3oc4cc(N(c5ccc(-c6ccccc6)cc5)c5ccc(-c6ccc(-c7ccccc7)cc6)cc5)ccc4c3c2)c2ccccc2-c2ccccc21 ; CC1(c2ccc3oc4cc(N(c5ccc(-c6ccccc6)cc5)c5cc(-c6ccccc6)cc(-c6ccccc6)c5)ccc4c3c2)c2ccccc2-c2ccccc21 ; CC1(c2ccc3oc4cc(N(c5ccc(-c6ccccc6)cc5)c5ccc6c7ccccc7c7ccccc7c6c5)ccc4c3c2)c2ccccc2-c2ccccc21]

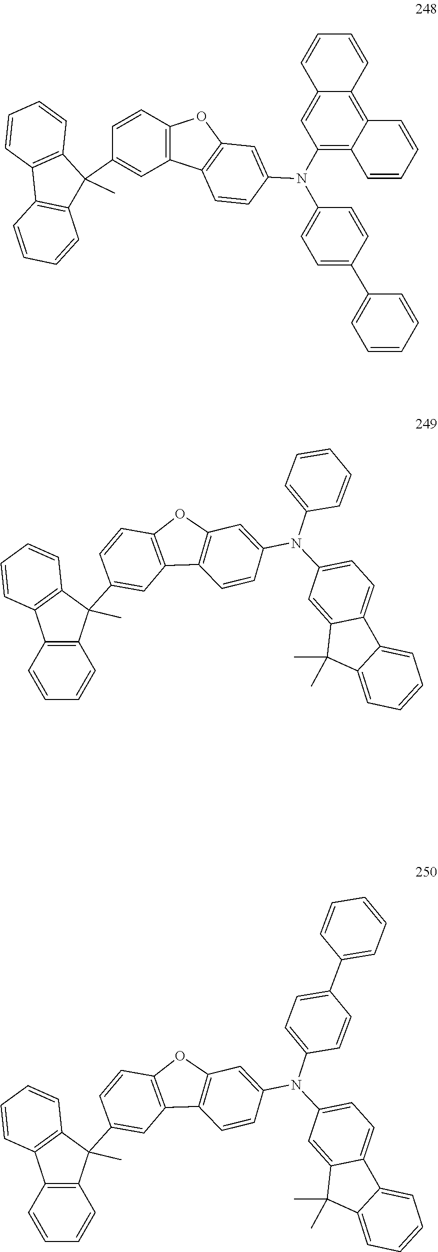 [SMILES: CC1(c2ccc3oc4cc(N(c5ccc(-c6ccccc6)cc5)c5cc6ccccc6c6ccccc56)ccc4c3c2)c2ccccc2-c2ccccc21 ; CC1(C)c2ccccc2-c2ccc(N(c3ccccc3)c3ccc4c(c3)oc3ccc(C5(C)c6ccccc6-c6ccccc65)cc34)cc21 ; CC1(C)c2ccccc2-c2ccc(N(c3ccc(-c4ccccc4)cc3)c3ccc4c(c3)oc3ccc(C5(C)c6ccccc6-c6ccccc65)cc34)cc21]

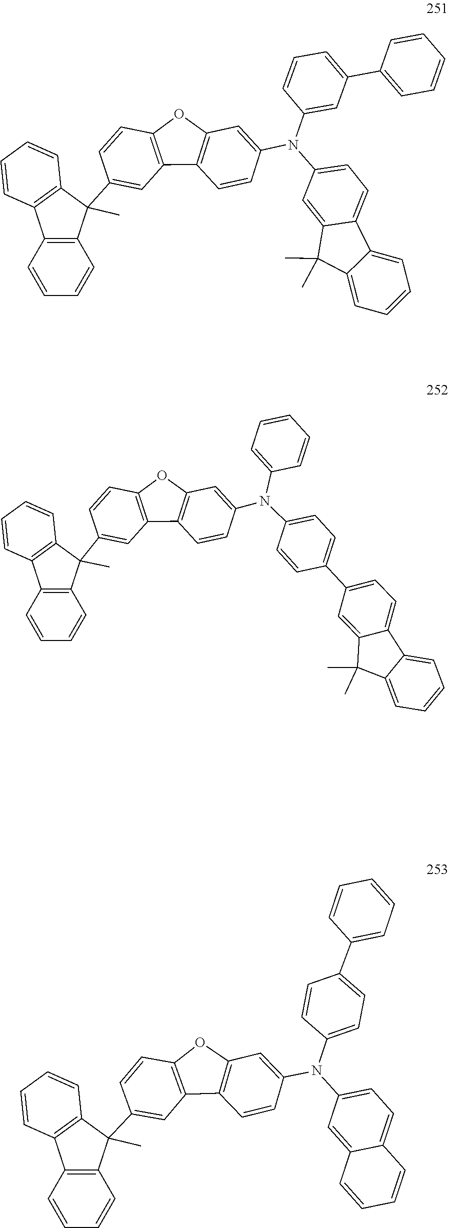 [SMILES: CC1(C)c2ccccc2-c2ccc(N(c3cccc(-c4ccccc4)c3)c3ccc4c(c3)oc3ccc(C5(C)c6ccccc6-c6ccccc65)cc34)cc21 ; CC1(C)c2ccccc2-c2ccc(-c3ccc(N(c4ccccc4)c4ccc5c(c4)oc4ccc(C6(C)c7ccccc7-c7ccccc76)cc45)cc3)cc21 ; CC1(c2ccc3oc4cc(N(c5ccc(-c6ccccc6)cc5)c5ccc6ccccc6c5)ccc4c3c2)c2ccccc2-c2ccccc21]

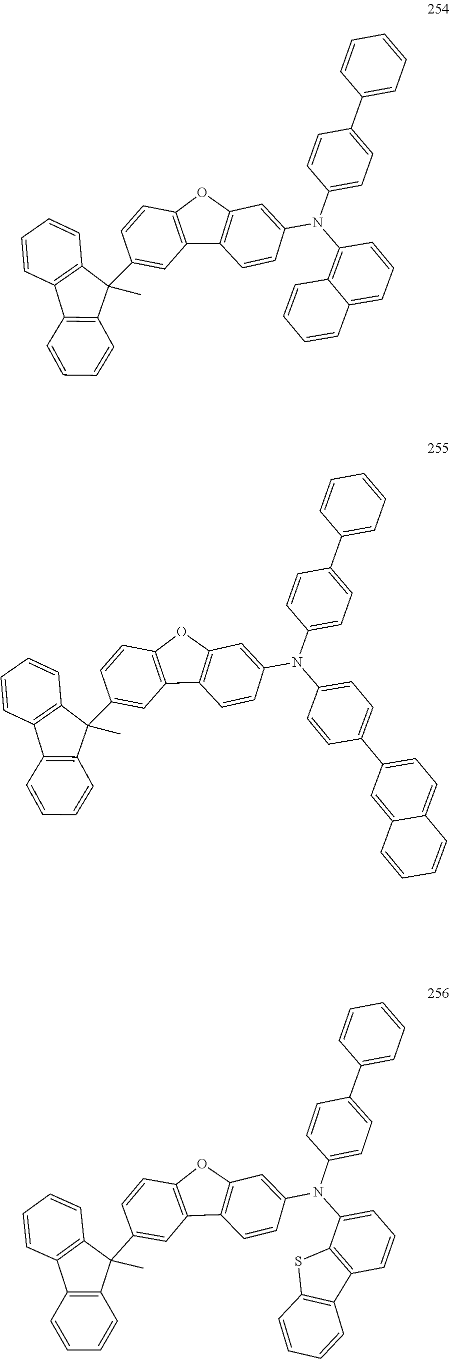 [SMILES: CC1(c2ccc3oc4cc(N(c5ccc(-c6ccccc6)cc5)c5cccc6ccccc56)ccc4c3c2)c2ccccc2-c2ccccc21 ; CC1(c2ccc3oc4cc(N(c5ccc(-c6ccccc6)cc5)c5ccc(-c6ccc7ccccc7c6)cc5)ccc4c3c2)c2ccccc2-c2ccccc21 ; CC1(c2ccc3oc4cc(N(c5ccc(-c6ccccc6)cc5)c5cccc6c5sc5ccccc56)ccc4c3c2)c2ccccc2-c2ccccc21]

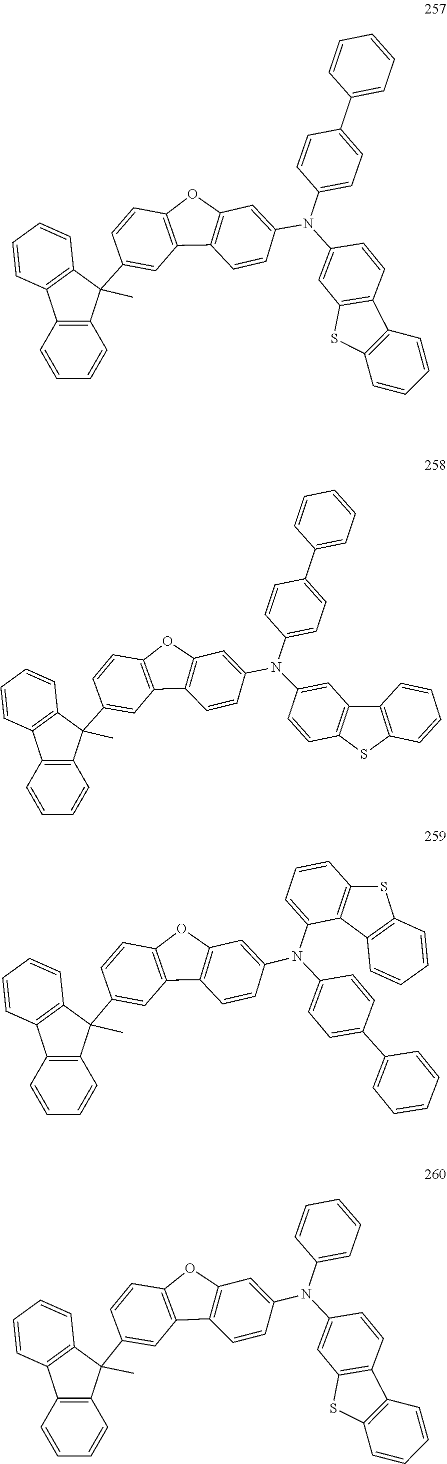 [SMILES: CC1(c2ccc3oc4cc(N(c5ccc(-c6ccccc6)cc5)c5ccc6c(c5)sc5ccccc56)ccc4c3c2)c2ccccc2-c2ccccc21 ; CC1(c2ccc3oc4cc(N(c5ccc(-c6ccccc6)cc5)c5ccc6sc7ccccc7c6c5)ccc4c3c2)c2ccccc2-c2ccccc21 ; CC1(c2ccc3oc4cc(N(c5ccc(-c6ccccc6)cc5)c5cccc6sc7ccccc7c56)ccc4c3c2)c2ccccc2-c2ccccc21 ; CC1(c2ccc3oc4cc(N(c5ccccc5)c5ccc6c(c5)sc5ccccc56)ccc4c3c2)c2ccccc2-c2ccccc21]

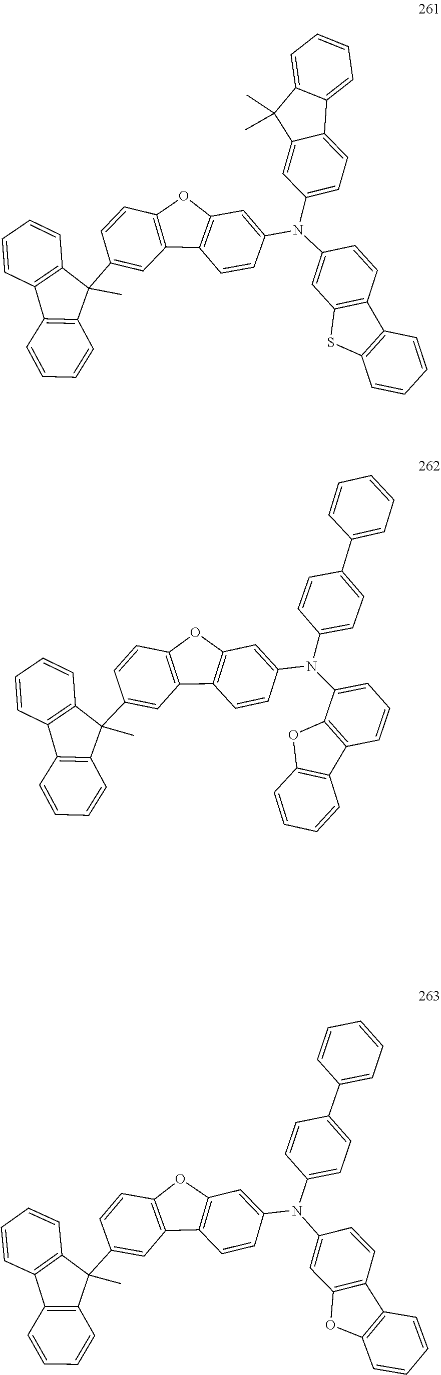 [SMILES: CC1(C)c2ccccc2-c2ccc(N(c3ccc4c(c3)oc3ccc(C5(C)c6ccccc6-c6ccccc65)cc34)c3ccc4c(c3)sc3ccccc34)cc21 ; CC1(c2ccc3oc4cc(N(c5ccc(-c6ccccc6)cc5)c5cccc6c5oc5ccccc56)ccc4c3c2)c2ccccc2-c2ccccc21 ; CC1(c2ccc3oc4cc(N(c5ccc(-c6ccccc6)cc5)c5ccc6c(c5)oc5ccccc56)ccc4c3c2)c2ccccc2-c2ccccc21]

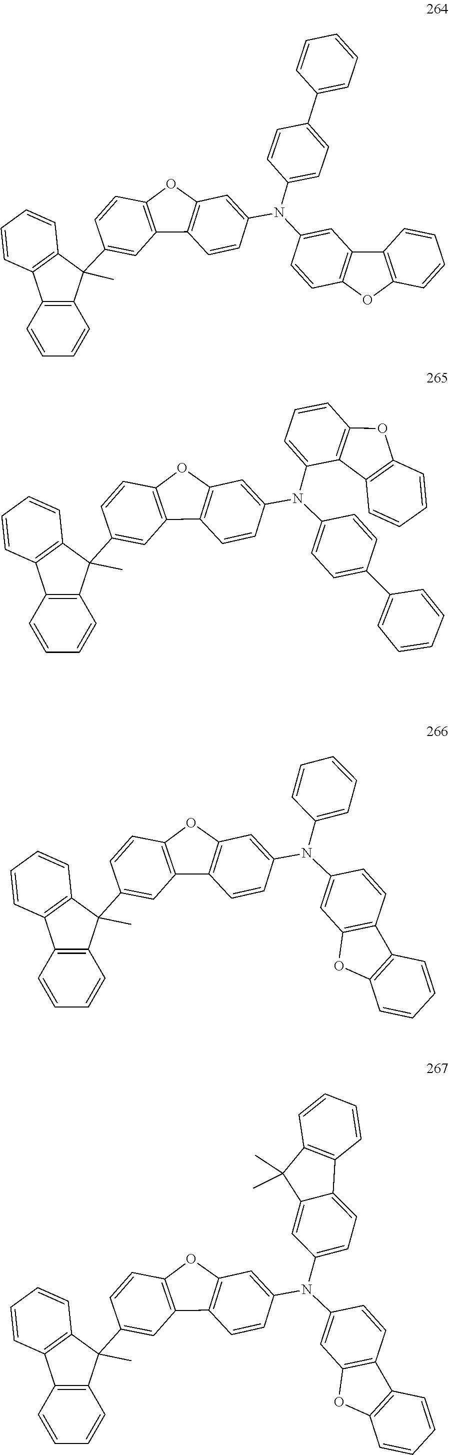 [SMILES: CC1(c2ccc3oc4cc(N(c5ccc(-c6ccccc6)cc5)c5ccc6oc7ccccc7c6c5)ccc4c3c2)c2ccccc2-c2ccccc21 ; CC1(c2ccc3oc4cc(N(c5ccc(-c6ccccc6)cc5)c5cccc6oc7ccccc7c56)ccc4c3c2)c2ccccc2-c2ccccc21 ; CC1(c2ccc3oc4cc(N(c5ccccc5)c5ccc6c(c5)oc5ccccc56)ccc4c3c2)c2ccccc2-c2ccccc21 ; CC1(C)c2ccccc2-c2ccc(N(c3ccc4c(c3)oc3ccccc34)c3ccc4c(c3)oc3ccc(C5(C)c6ccccc6-c6ccccc65)cc34)cc21]

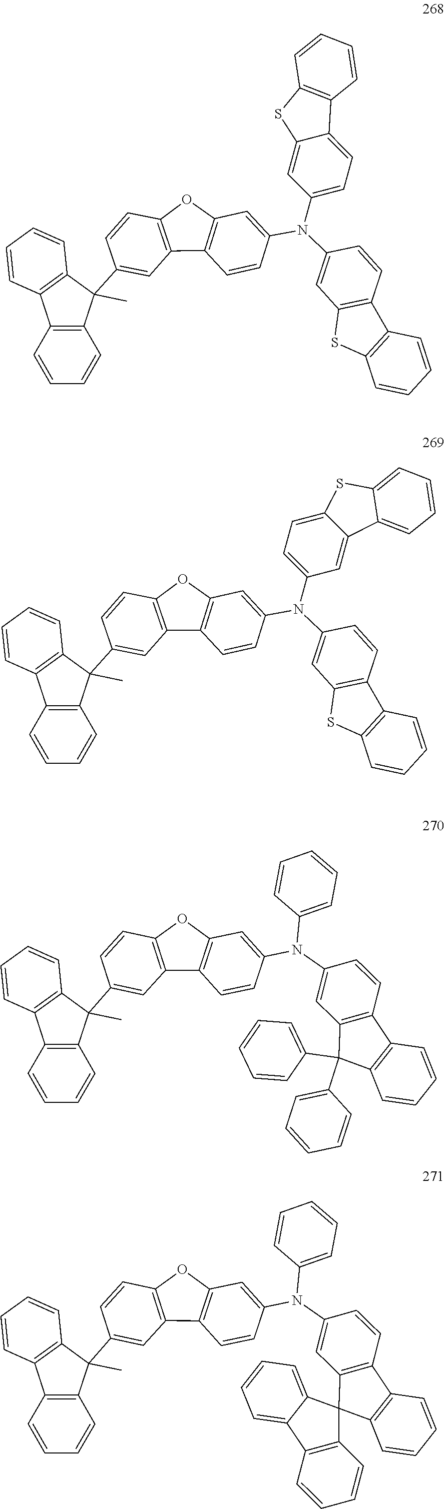 [SMILES: CC1(c2ccc3oc4cc(N(c5ccc6c(c5)sc5ccccc56)c5ccc6c(c5)sc5ccccc56)ccc4c3c2)c2ccccc2-c2ccccc21 ; CC1(c2ccc3oc4cc(N(c5ccc6c(c5)sc5ccccc56)c5ccc6sc7ccccc7c6c5)ccc4c3c2)c2ccccc2-c2ccccc21 ; CC1(c2ccc3oc4cc(N(c5ccccc5)c5ccc6c(c5)C(c5ccccc5)(c5ccccc5)c5ccccc5-6)ccc4c3c2)c2ccccc2-c2ccccc21 ; CC1(c2ccc3oc4cc(N(c5ccccc5)c5ccc6c(c5)C5(c7ccccc7-c7ccccc75)c5ccccc5-6)ccc4c3c2)c2ccccc2-c2ccccc21]

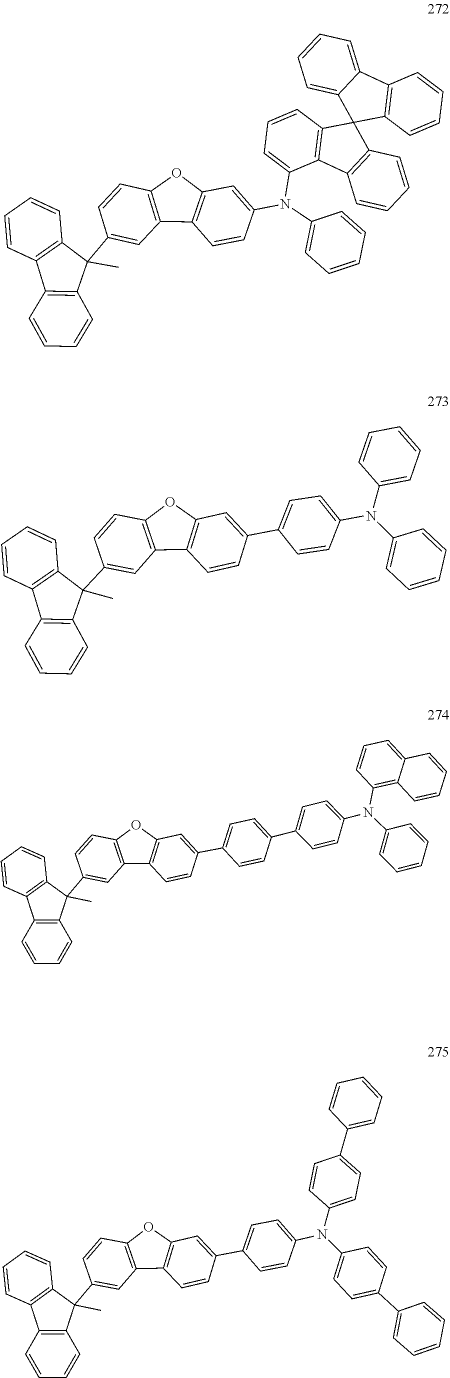 [SMILES: CC1(c2ccc3oc4cc(N(c5ccccc5)c5cccc6c5-c5ccccc5C65c6ccccc6-c6ccccc65)ccc4c3c2)c2ccccc2-c2ccccc21 ; CC1(c2ccc3oc4cc(-c5ccc(N(c6ccccc6)c6ccccc6)cc5)ccc4c3c2)c2ccccc2-c2ccccc21 ; CC1(c2ccc3oc4cc(-c5ccc(-c6ccc(N(c7ccccc7)c7cccc8ccccc78)cc6)cc5)ccc4c3c2)c2ccccc2-c2ccccc21 ; CC1(c2ccc3oc4cc(-c5ccc(N(c6ccc(-c7ccccc7)cc6)c6ccc(-c7ccccc7)cc6)cc5)ccc4c3c2)c2ccccc2-c2ccccc21]

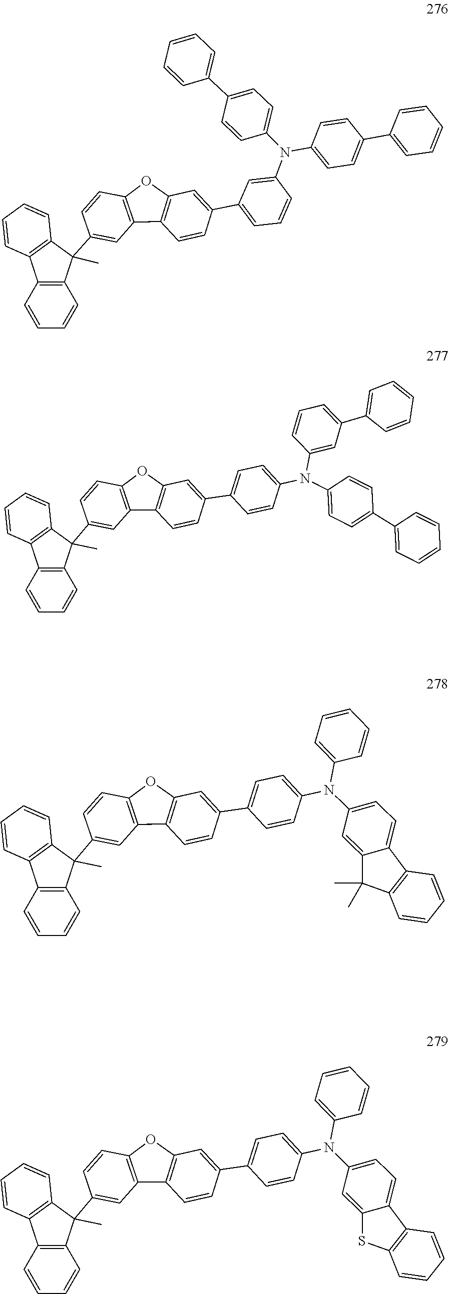 [SMILES: CC1(c2ccc3oc4cc(-c5cccc(N(c6ccc(-c7ccccc7)cc6)c6ccc(-c7ccccc7)cc6)c5)ccc4c3c2)c2ccccc2-c2ccccc21 ; CC1(c2ccc3oc4cc(-c5ccc(N(c6ccc(-c7ccccc7)cc6)c6cccc(-c7ccccc7)c6)cc5)ccc4c3c2)c2ccccc2-c2ccccc21 ; CC1(C)c2ccccc2-c2ccc(N(c3ccccc3)c3ccc(-c4ccc5c(c4)oc4ccc(C6(C)c7ccccc7-c7ccccc76)cc45)cc3)cc21 ; CC1(c2ccc3oc4cc(-c5ccc(N(c6ccccc6)c6ccc7c(c6)sc6ccccc67)cc5)ccc4c3c2)c2ccccc2-c2ccccc21]

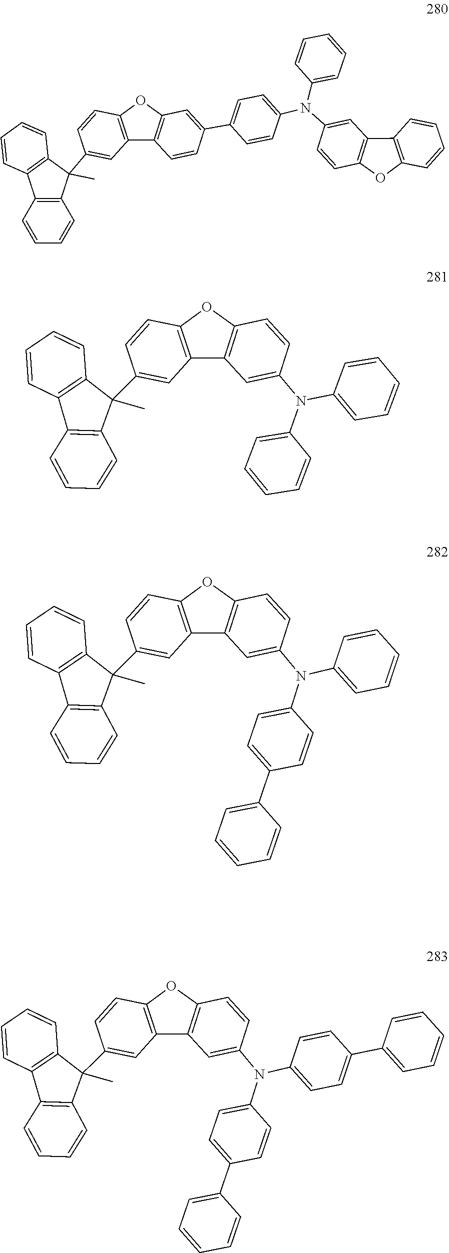 [SMILES: CC1(c2ccc3oc4cc(-c5ccc(N(c6ccccc6)c6ccc7oc8ccccc8c7c6)cc5)ccc4c3c2)c2ccccc2-c2ccccc21 ; CC1(c2ccc3oc4ccc(N(c5ccccc5)c5ccccc5)cc4c3c2)c2ccccc2-c2ccccc21 ; CC1(c2ccc3oc4ccc(N(c5ccccc5)c5ccc(-c6ccccc6)cc5)cc4c3c2)c2ccccc2-c2ccccc21 ; CC1(c2ccc3oc4ccc(N(c5ccc(-c6ccccc6)cc5)c5ccc(-c6ccccc6)cc5)cc4c3c2)c2ccccc2-c2ccccc21]

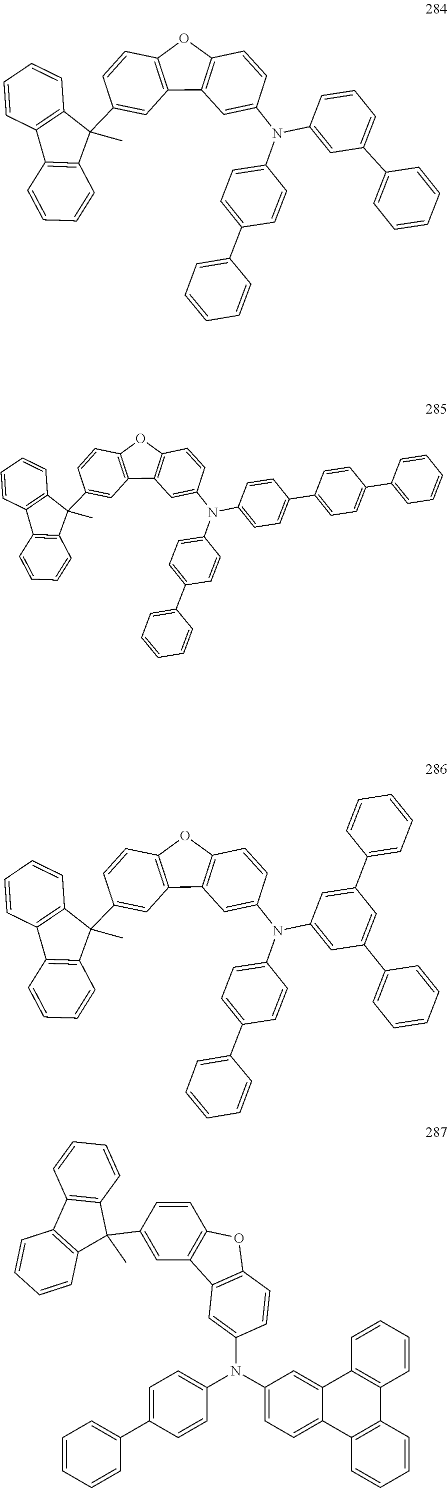 [SMILES: CC1(c2ccc3oc4ccc(N(c5ccc(-c6ccccc6)cc5)c5cccc(-c6ccccc6)c5)cc4c3c2)c2ccccc2-c2ccccc21 ; CC1(c2ccc3oc4ccc(N(c5ccc(-c6ccccc6)cc5)c5ccc(-c6ccc(-c7ccccc7)cc6)cc5)cc4c3c2)c2ccccc2-c2ccccc21 ; CC1(c2ccc3oc4ccc(N(c5ccc(-c6ccccc6)cc5)c5cc(-c6ccccc6)cc(-c6ccccc6)c5)cc4c3c2)c2ccccc2-c2ccccc21 ; CC1(c2ccc3oc4ccc(N(c5ccc(-c6ccccc6)cc5)c5ccc6c7ccccc7c7ccccc7c6c5)cc4c3c2)c2ccccc2-c2ccccc21]

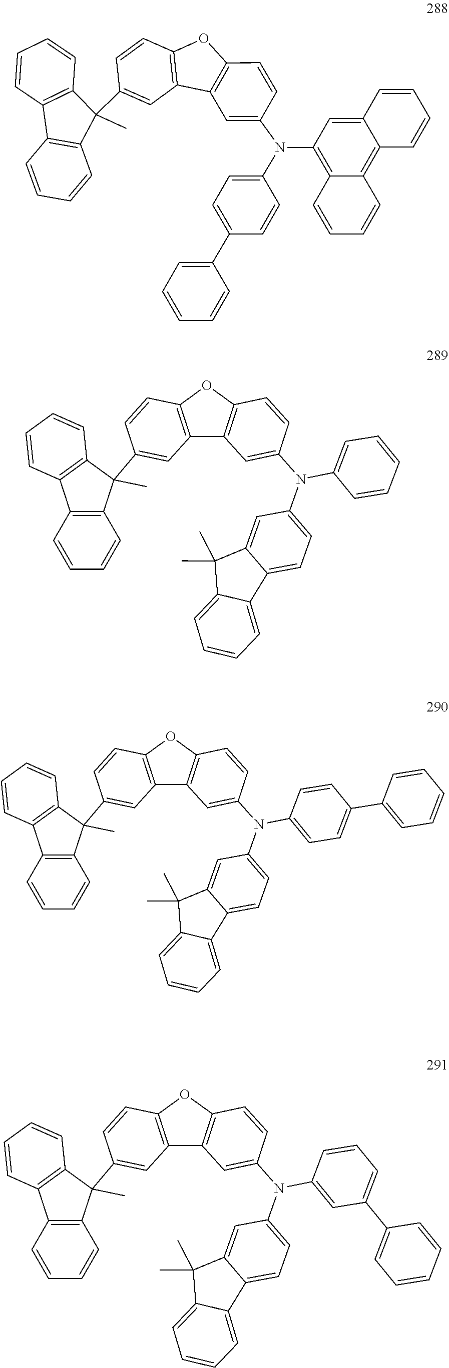 [SMILES: CC1(c2ccc3oc4ccc(N(c5ccc(-c6ccccc6)cc5)c5cc6ccccc6c6ccccc56)cc4c3c2)c2ccccc2-c2ccccc21 ; CC1(C)c2ccccc2-c2ccc(N(c3ccccc3)c3ccc4oc5ccc(C6(C)c7ccccc7-c7ccccc76)cc5c4c3)cc21 ; CC1(C)c2ccccc2-c2ccc(N(c3ccc(-c4ccccc4)cc3)c3ccc4oc5ccc(C6(C)c7ccccc7-c7ccccc76)cc5c4c3)cc21 ; CC1(C)c2ccccc2-c2ccc(N(c3cccc(-c4ccccc4)c3)c3ccc4oc5ccc(C6(C)c7ccccc7-c7ccccc76)cc5c4c3)cc21]

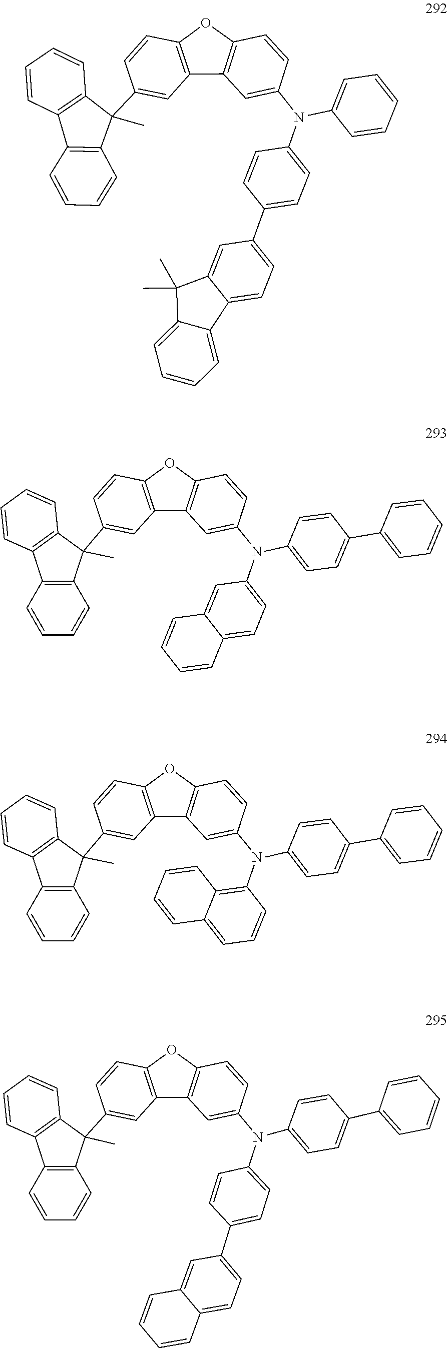 [SMILES: CC1(C)c2ccccc2-c2ccc(-c3ccc(N(c4ccccc4)c4ccc5oc6ccc(C7(C)c8ccccc8-c8ccccc87)cc6c5c4)cc3)cc21 ; CC1(c2ccc3oc4ccc(N(c5ccc(-c6ccccc6)cc5)c5ccc6ccccc6c5)cc4c3c2)c2ccccc2-c2ccccc21 ; CC1(c2ccc3oc4ccc(N(c5ccc(-c6ccccc6)cc5)c5cccc6ccccc56)cc4c3c2)c2ccccc2-c2ccccc21 ; CC1(c2ccc3oc4ccc(N(c5ccc(-c6ccccc6)cc5)c5ccc(-c6ccc7ccccc7c6)cc5)cc4c3c2)c2ccccc2-c2ccccc21]

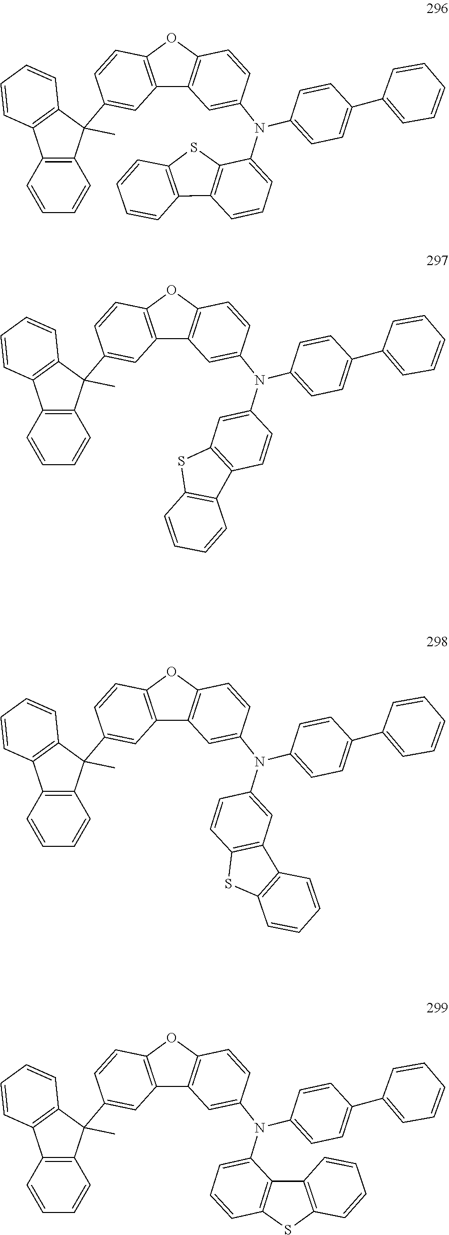 [SMILES: CC1(c2ccc3oc4ccc(N(c5ccc(-c6ccccc6)cc5)c5cccc6c5sc5ccccc56)cc4c3c2)c2ccccc2-c2ccccc21 ; CC1(c2ccc3oc4ccc(N(c5ccc(-c6ccccc6)cc5)c5ccc6c(c5)sc5ccccc56)cc4c3c2)c2ccccc2-c2ccccc21 ; CC1(c2ccc3oc4ccc(N(c5ccc(-c6ccccc6)cc5)c5ccc6sc7ccccc7c6c5)cc4c3c2)c2ccccc2-c2ccccc21 ; CC1(c2ccc3oc4ccc(N(c5ccc(-c6ccccc6)cc5)c5cccc6sc7ccccc7c56)cc4c3c2)c2ccccc2-c2ccccc21]

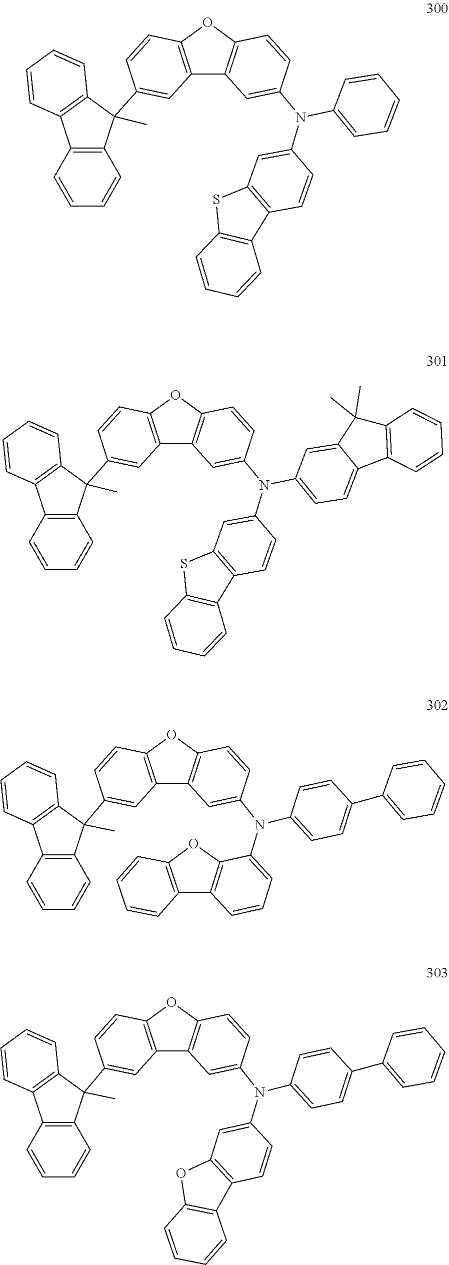 [SMILES: CC1(c2ccc3oc4ccc(N(c5ccccc5)c5ccc6c(c5)sc5ccccc56)cc4c3c2)c2ccccc2-c2ccccc21 ; CC1(C)c2ccccc2-c2ccc(N(c3ccc4c(c3)sc3ccccc34)c3ccc4oc5ccc(C6(C)c7ccccc7-c7ccccc76)cc5c4c3)cc21 ; CC1(c2ccc3oc4ccc(N(c5ccc(-c6ccccc6)cc5)c5cccc6c5oc5ccccc56)cc4c3c2)c2ccccc2-c2ccccc21 ; CC1(c2ccc3oc4ccc(N(c5ccc(-c6ccccc6)cc5)c5ccc6c(c5)oc5ccccc56)cc4c3c2)c2ccccc2-c2ccccc21]

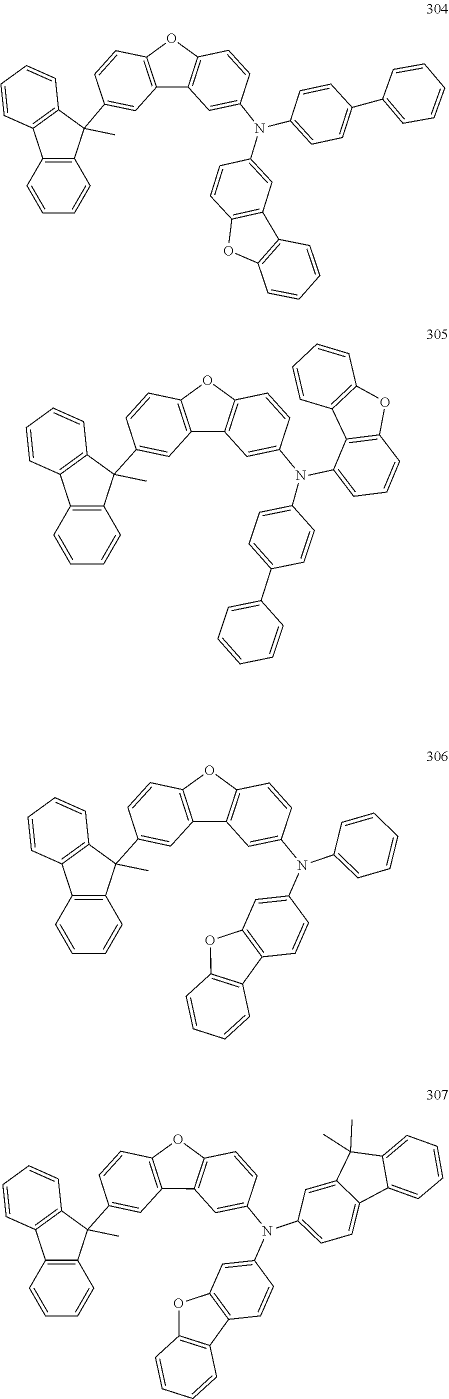 [SMILES: CC1(c2ccc3oc4ccc(N(c5ccc(-c6ccccc6)cc5)c5ccc6oc7ccccc7c6c5)cc4c3c2)c2ccccc2-c2ccccc21 ; CC1(c2ccc3oc4ccc(N(c5ccc(-c6ccccc6)cc5)c5cccc6oc7ccccc7c56)cc4c3c2)c2ccccc2-c2ccccc21 ; CC1(c2ccc3oc4ccc(N(c5ccccc5)c5ccc6c(c5)oc5ccccc56)cc4c3c2)c2ccccc2-c2ccccc21 ; CC1(C)c2ccccc2-c2ccc(N(c3ccc4c(c3)oc3ccccc34)c3ccc4oc5ccc(C6(C)c7ccccc7-c7ccccc76)cc5c4c3)cc21]

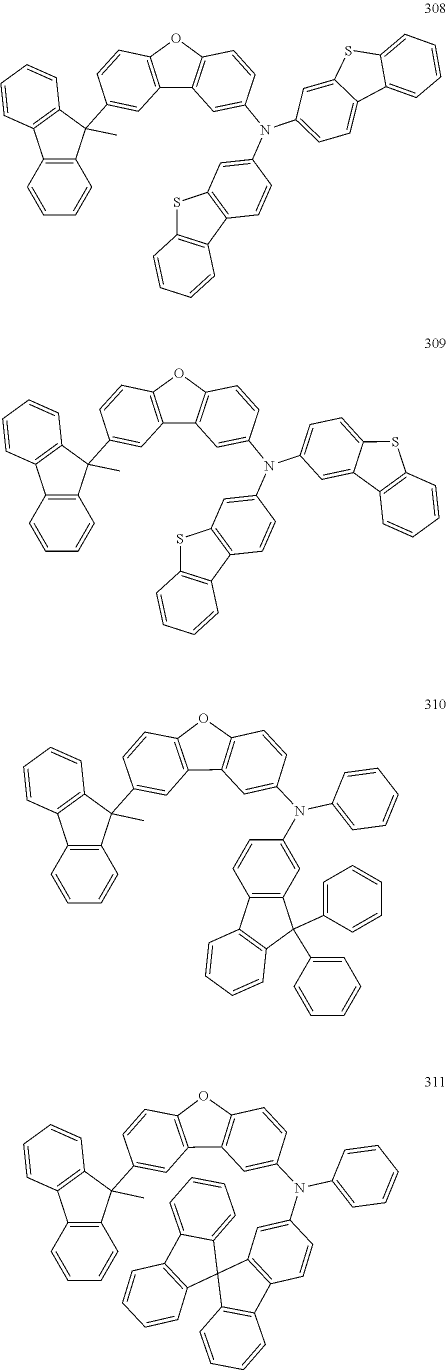 [SMILES: CC1(c2ccc3oc4ccc(N(c5ccc6c(c5)sc5ccccc56)c5ccc6c(c5)sc5ccccc56)cc4c3c2)c2ccccc2-c2ccccc21 ; CC1(c2ccc3oc4ccc(N(c5ccc6c(c5)sc5ccccc56)c5ccc6sc7ccccc7c6c5)cc4c3c2)c2ccccc2-c2ccccc21 ; CC1(c2ccc3oc4ccc(N(c5ccccc5)c5ccc6c(c5)C(c5ccccc5)(c5ccccc5)c5ccccc5-6)cc4c3c2)c2ccccc2-c2ccccc21 ; CC1(c2ccc3oc4ccc(N(c5ccccc5)c5ccc6c(c5)C5(c7ccccc7-c7ccccc75)c5ccccc5-6)cc4c3c2)c2ccccc2-c2ccccc21]

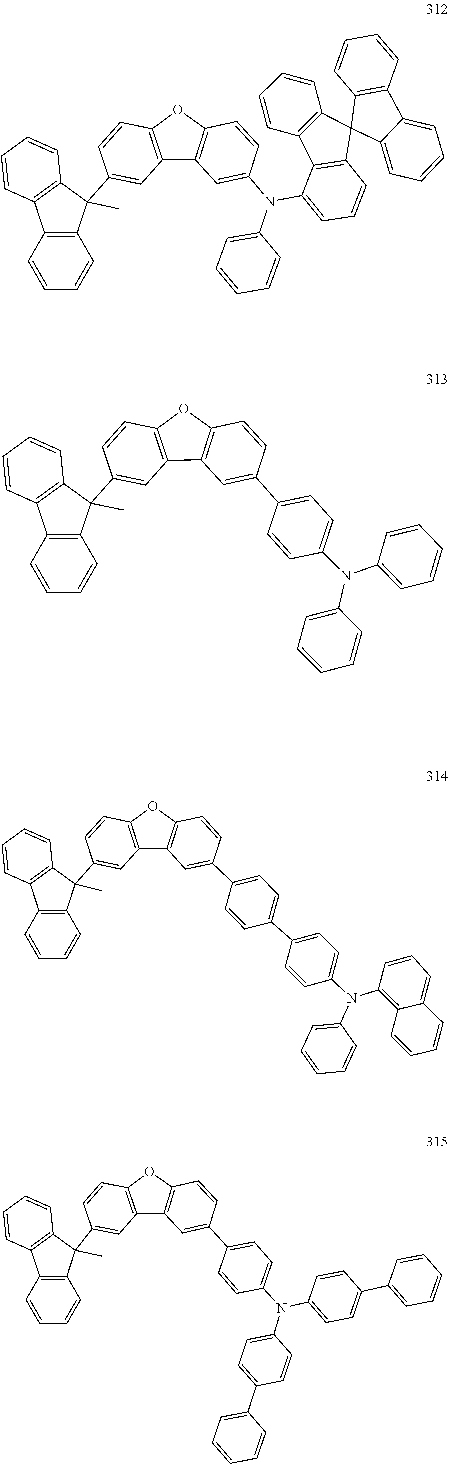 [SMILES: CC1(c2ccc3oc4ccc(N(c5ccccc5)c5cccc6c5-c5ccccc5C65c6ccccc6-c6ccccc65)cc4c3c2)c2ccccc2-c2ccccc21 ; CC1(c2ccc3oc4ccc(-c5ccc(N(c6ccccc6)c6ccccc6)cc5)cc4c3c2)c2ccccc2-c2ccccc21 ; CC1(c2ccc3oc4ccc(-c5ccc(-c6ccc(N(c7ccccc7)c7cccc8ccccc78)cc6)cc5)cc4c3c2)c2ccccc2-c2ccccc21 ; CC1(c2ccc3oc4ccc(-c5ccc(N(c6ccc(-c7ccccc7)cc6)c6ccc(-c7ccccc7)cc6)cc5)cc4c3c2)c2ccccc2-c2ccccc21]

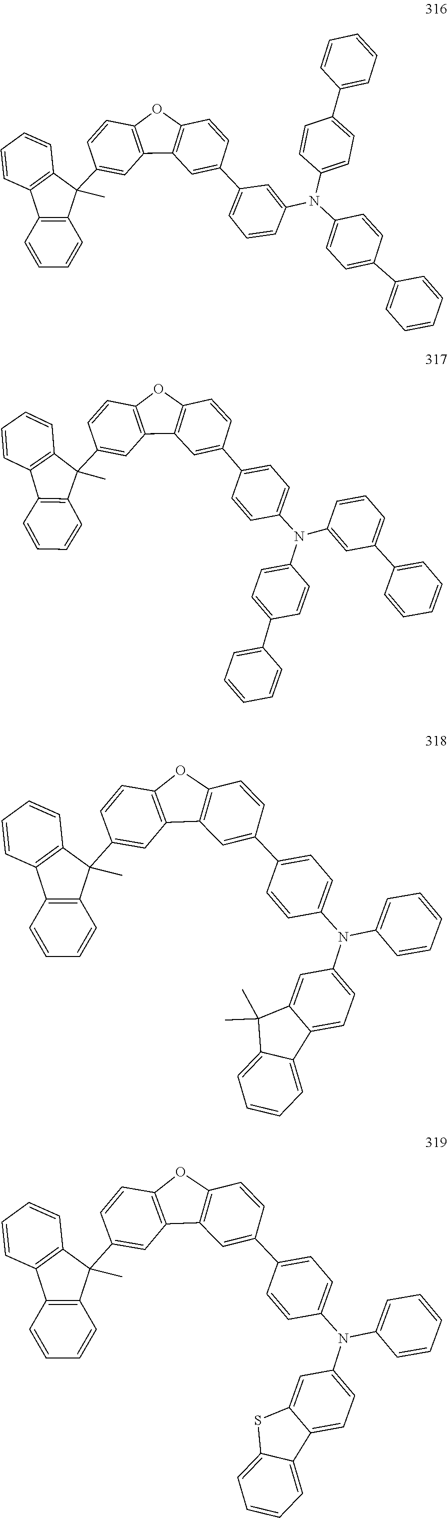 [SMILES: CC1(c2ccc3oc4ccc(-c5cccc(N(c6ccc(-c7ccccc7)cc6)c6ccc(-c7ccccc7)cc6)c5)cc4c3c2)c2ccccc2-c2ccccc21 ; CC1(c2ccc3oc4ccc(-c5ccc(N(c6ccc(-c7ccccc7)cc6)c6cccc(-c7ccccc7)c6)cc5)cc4c3c2)c2ccccc2-c2ccccc21 ; CC1(C)c2ccccc2-c2ccc(N(c3ccccc3)c3ccc(-c4ccc5oc6ccc(C7(C)c8ccccc8-c8ccccc87)cc6c5c4)cc3)cc21 ; CC1(c2ccc3oc4ccc(-c5ccc(N(c6ccccc6)c6ccc7c(c6)sc6ccccc67)cc5)cc4c3c2)c2ccccc2-c2ccccc21]

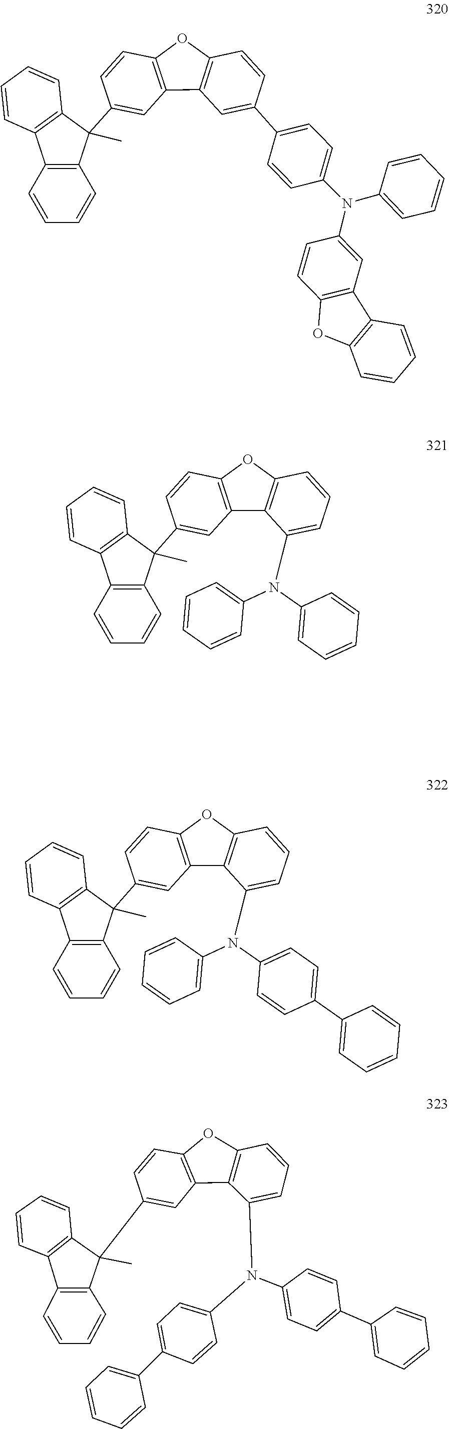 [SMILES: CC1(c2ccc3oc4ccc(-c5ccc(N(c6ccccc6)c6ccc7oc8ccccc8c7c6)cc5)cc4c3c2)c2ccccc2-c2ccccc21 ; CC1(c2ccc3oc4cccc(N(c5ccccc5)c5ccccc5)c4c3c2)c2ccccc2-c2ccccc21 ; CC1(c2ccc3oc4cccc(N(c5ccccc5)c5ccc(-c6ccccc6)cc5)c4c3c2)c2ccccc2-c2ccccc21 ; CC1(c2ccc3oc4cccc(N(c5ccc(-c6ccccc6)cc5)c5ccc(-c6ccccc6)cc5)c4c3c2)c2ccccc2-c2ccccc21]

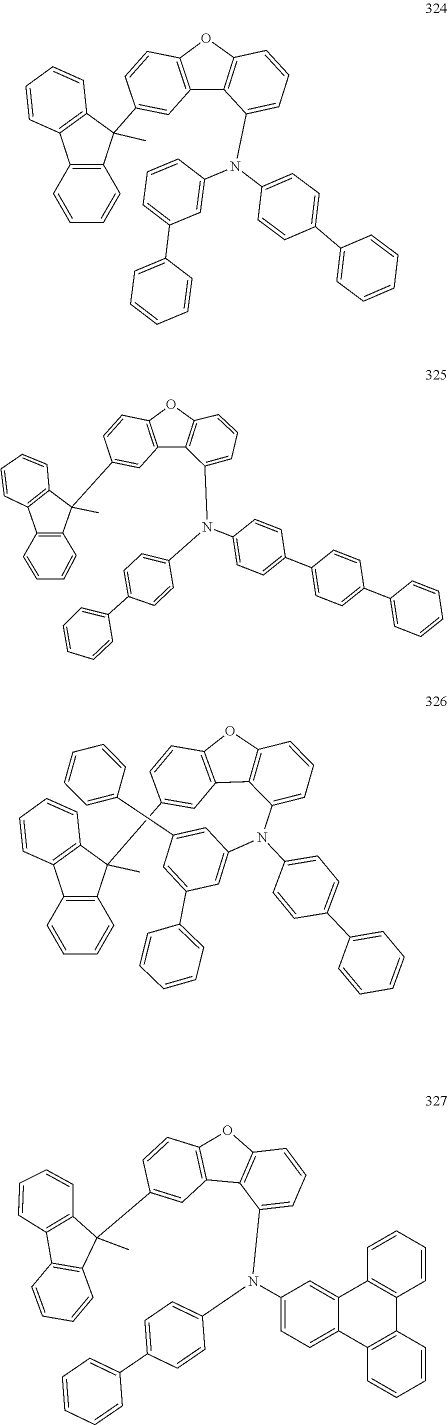 [SMILES: CC1(c2ccc3oc4cccc(N(c5ccc(-c6ccccc6)cc5)c5cccc(-c6ccccc6)c5)c4c3c2)c2ccccc2-c2ccccc21 ; CC1(c2ccc3oc4cccc(N(c5ccc(-c6ccccc6)cc5)c5ccc(-c6ccc(-c7ccccc7)cc6)cc5)c4c3c2)c2ccccc2-c2ccccc21 ; CC1(c2ccc3oc4cccc(N(c5ccc(-c6ccccc6)cc5)c5cc(-c6ccccc6)cc(-c6ccccc6)c5)c4c3c2)c2ccccc2-c2ccccc21 ; CC1(c2ccc3oc4cccc(N(c5ccc(-c6ccccc6)cc5)c5ccc6c7ccccc7c7ccccc7c6c5)c4c3c2)c2ccccc2-c2ccccc21]

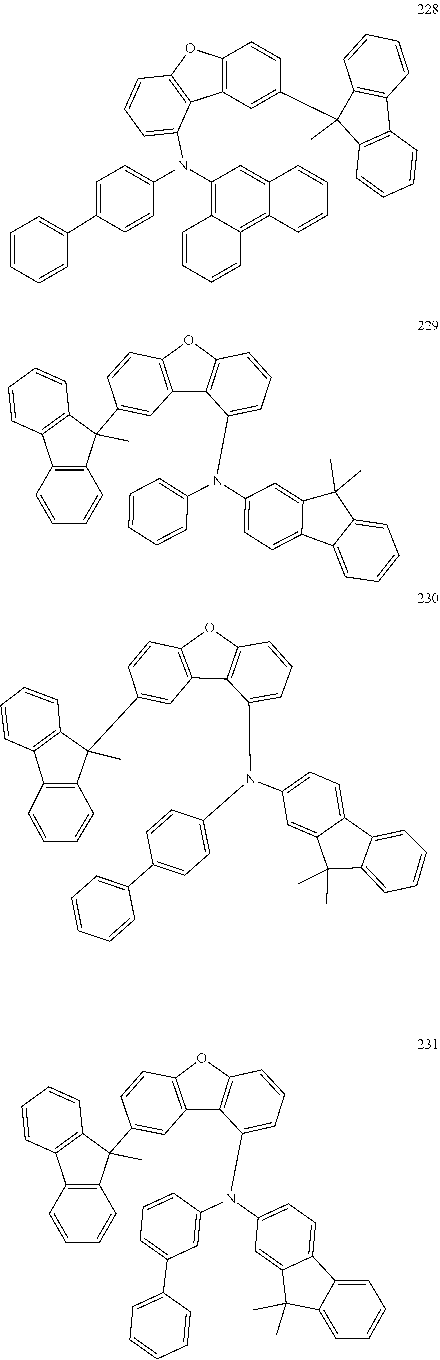 [SMILES: CC1(c2ccc3oc4cccc(N(c5ccc(-c6ccccc6)cc5)c5cc6ccccc6c6ccccc56)c4c3c2)c2ccccc2-c2ccccc21 ; CC1(C)c2ccccc2-c2ccc(N(c3ccccc3)c3cccc4oc5ccc(C6(C)c7ccccc7-c7ccccc76)cc5c34)cc21 ; CC1(C)c2ccccc2-c2ccc(N(c3ccc(-c4ccccc4)cc3)c3cccc4oc5ccc(C6(C)c7ccccc7-c7ccccc76)cc5c34)cc21 ; CC1(C)c2ccccc2-c2ccc(N(c3cccc(-c4ccccc4)c3)c3cccc4oc5ccc(C6(C)c7ccccc7-c7ccccc76)cc5c34)cc21]

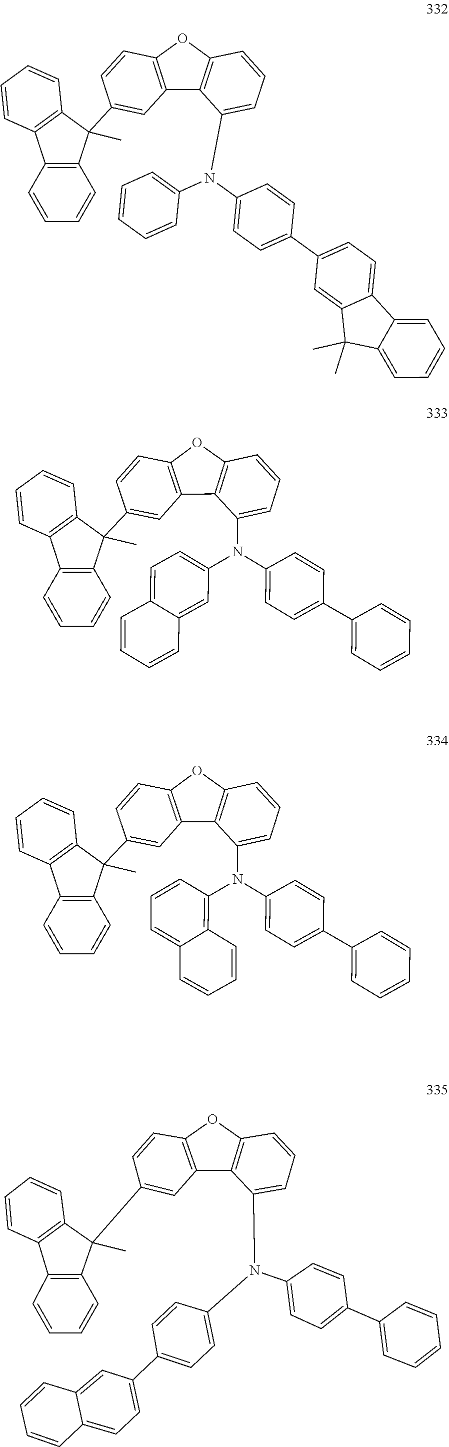 [SMILES: CC1(C)c2ccccc2-c2ccc(-c3ccc(N(c4ccccc4)c4cccc5oc6ccc(C7(C)c8ccccc8-c8ccccc87)cc6c45)cc3)cc21 ; CC1(c2ccc3oc4cccc(N(c5ccc(-c6ccccc6)cc5)c5ccc6ccccc6c5)c4c3c2)c2ccccc2-c2ccccc21 ; CC1(c2ccc3oc4cccc(N(c5ccc(-c6ccccc6)cc5)c5cccc6ccccc56)c4c3c2)c2ccccc2-c2ccccc21 ; CC1(c2ccc3oc4cccc(N(c5ccc(-c6ccccc6)cc5)c5ccc(-c6ccc7ccccc7c6)cc5)c4c3c2)c2ccccc2-c2ccccc21]

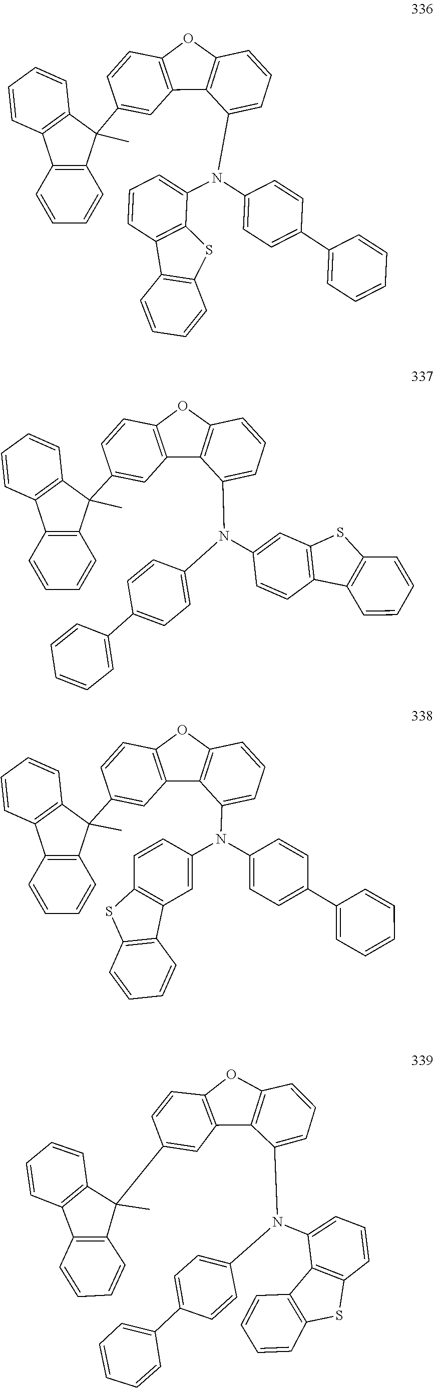 [SMILES: CC1(c2ccc3oc4cccc(N(c5ccc(-c6ccccc6)cc5)c5cccc6c5sc5ccccc56)c4c3c2)c2ccccc2-c2ccccc21 ; CC1(c2ccc3oc4cccc(N(c5ccc(-c6ccccc6)cc5)c5ccc6c(c5)sc5ccccc56)c4c3c2)c2ccccc2-c2ccccc21 ; CC1(c2ccc3oc4cccc(N(c5ccc(-c6ccccc6)cc5)c5ccc6sc7ccccc7c6c5)c4c3c2)c2ccccc2-c2ccccc21 ; CC1(c2ccc3oc4cccc(N(c5ccc(-c6ccccc6)cc5)c5cccc6sc7ccccc7c56)c4c3c2)c2ccccc2-c2ccccc21]

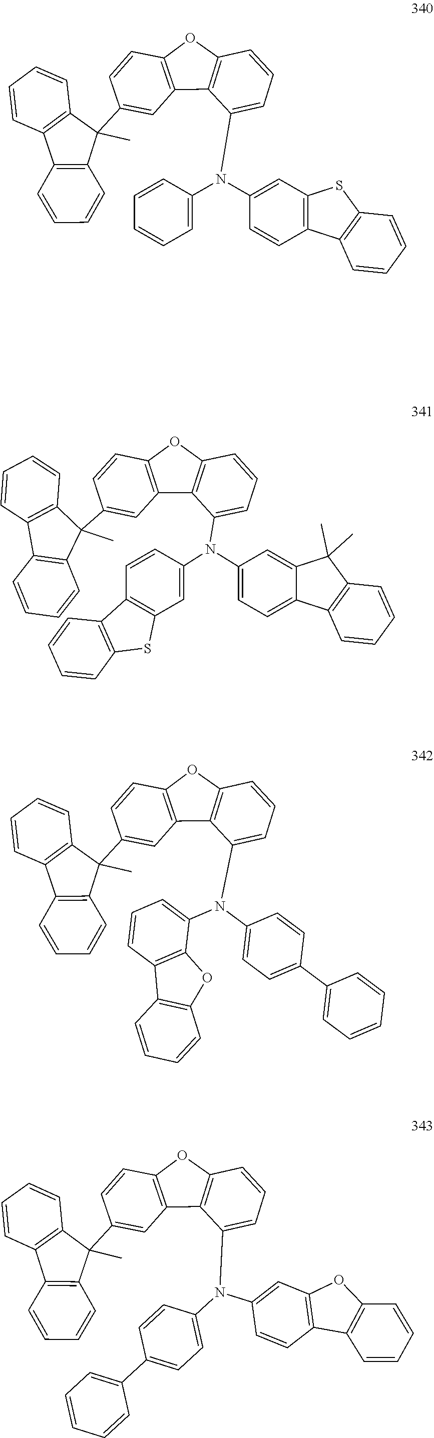 [SMILES: CC1(c2ccc3oc4cccc(N(c5ccccc5)c5ccc6c(c5)sc5ccccc56)c4c3c2)c2ccccc2-c2ccccc21 ; CC1(C)c2ccccc2-c2ccc(N(c3ccc4c(c3)sc3ccccc34)c3cccc4oc5ccc(C6(C)c7ccccc7-c7ccccc76)cc5c34)cc21 ; CC1(c2ccc3oc4cccc(N(c5ccc(-c6ccccc6)cc5)c5cccc6c5oc5ccccc56)c4c3c2)c2ccccc2-c2ccccc21 ; CC1(c2ccc3oc4cccc(N(c5ccc(-c6ccccc6)cc5)c5ccc6c(c5)oc5ccccc56)c4c3c2)c2ccccc2-c2ccccc21]

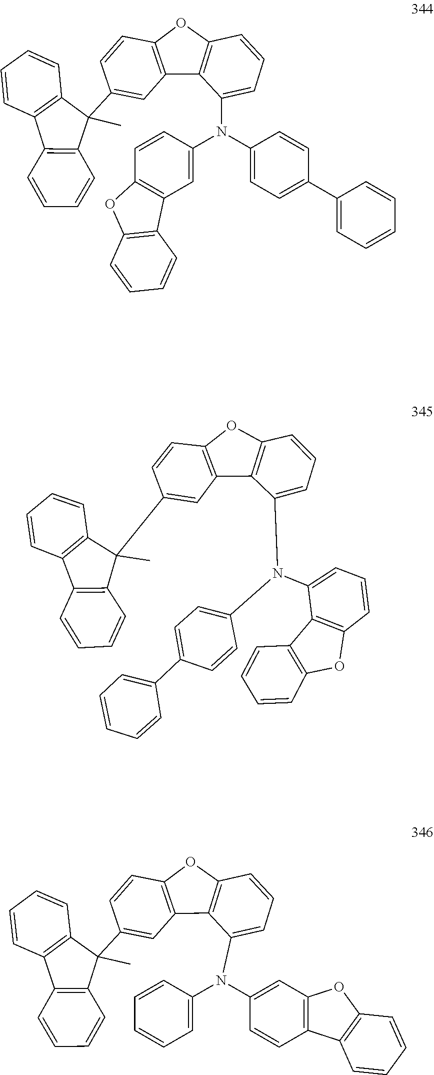 [SMILES: CC1(c2ccc3oc4cccc(N(c5ccc(-c6ccccc6)cc5)c5ccc6oc7ccccc7c6c5)c4c3c2)c2ccccc2-c2ccccc21 ; CC1(c2ccc3oc4cccc(N(c5ccc(-c6ccccc6)cc5)c5cccc6oc7ccccc7c56)c4c3c2)c2ccccc2-c2ccccc21 ; CC1(c2ccc3oc4cccc(N(c5ccccc5)c5ccc6c(c5)oc5ccccc56)c4c3c2)c2ccccc2-c2ccccc21]

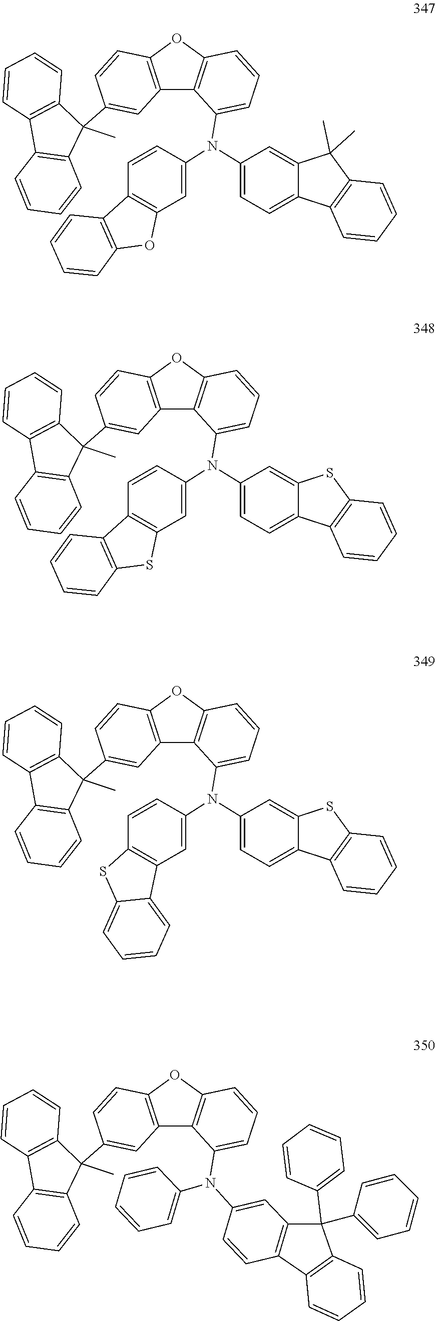 [SMILES: CC1(C)c2ccccc2-c2ccc(N(c3ccc4c(c3)oc3ccccc34)c3cccc4oc5ccc(C6(C)c7ccccc7-c7ccccc76)cc5c34)cc21 ; CC1(c2ccc3oc4cccc(N(c5ccc6c(c5)sc5ccccc56)c5ccc6c(c5)sc5ccccc56)c4c3c2)c2ccccc2-c2ccccc21 ; CC1(c2ccc3oc4cccc(N(c5ccc6c(c5)sc5ccccc56)c5ccc6sc7ccccc7c6c5)c4c3c2)c2ccccc2-c2ccccc21 ; CC1(c2ccc3oc4cccc(N(c5ccccc5)c5ccc6c(c5)C(c5ccccc5)(c5ccccc5)c5ccccc5-6)c4c3c2)c2ccccc2-c2ccccc21]

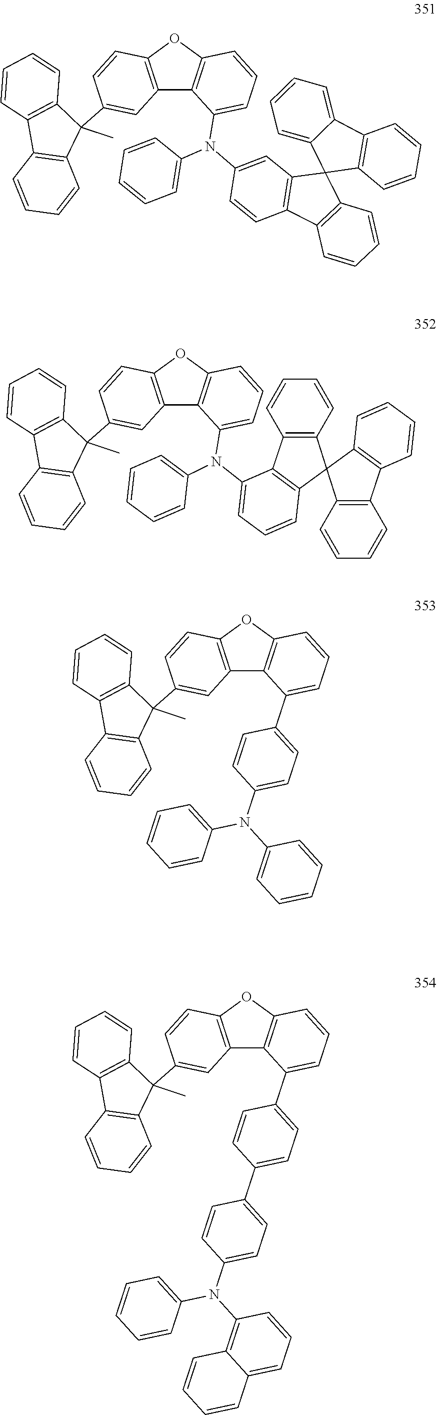 [SMILES: CC1(c2ccc3oc4cccc(N(c5ccccc5)c5ccc6c(c5)C5(c7ccccc7-c7ccccc75)c5ccccc5-6)c4c3c2)c2ccccc2-c2ccccc21 ; CC1(c2ccc3oc4cccc(N(c5ccccc5)c5cccc6c5-c5ccccc5C65c6ccccc6-c6ccccc65)c4c3c2)c2ccccc2-c2ccccc21 ; CC1(c2ccc3oc4cccc(-c5ccc(N(c6ccccc6)c6ccccc6)cc5)c4c3c2)c2ccccc2-c2ccccc21 ; CC1(c2ccc3oc4cccc(-c5ccc(-c6ccc(N(c7ccccc7)c7cccc8ccccc78)cc6)cc5)c4c3c2)c2ccccc2-c2ccccc21]

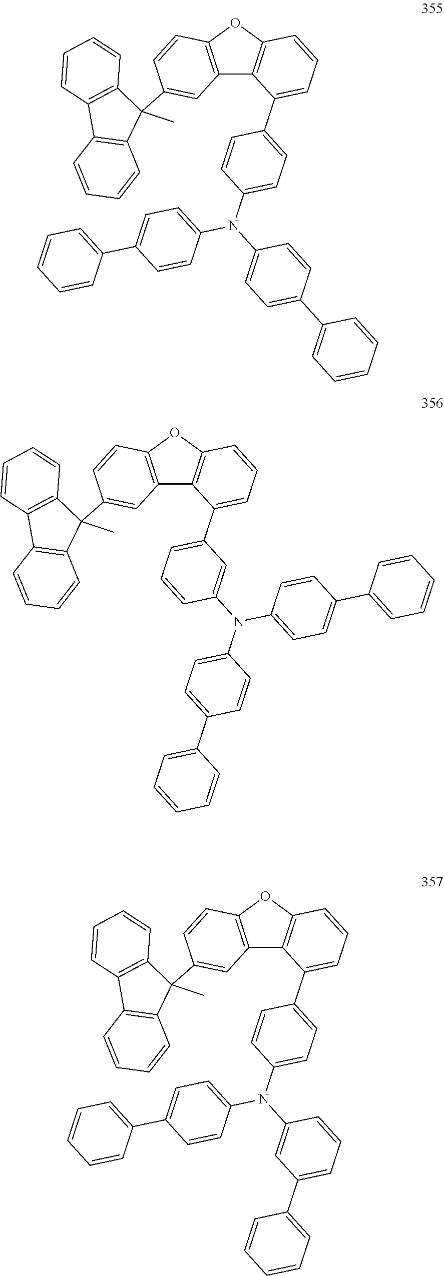 [SMILES: CC1(c2ccc3oc4cccc(-c5ccc(N(c6ccc(-c7ccccc7)cc6)c6ccc(-c7ccccc7)cc6)cc5)c4c3c2)c2ccccc2-c2ccccc21 ; CC1(c2ccc3oc4cccc(-c5cccc(N(c6ccc(-c7ccccc7)cc6)c6ccc(-c7ccccc7)cc6)c5)c4c3c2)c2ccccc2-c2ccccc21 ; CC1(c2ccc3oc4cccc(-c5ccc(N(c6ccc(-c7ccccc7)cc6)c6cccc(-c7ccccc7)c6)cc5)c4c3c2)c2ccccc2-c2ccccc21]

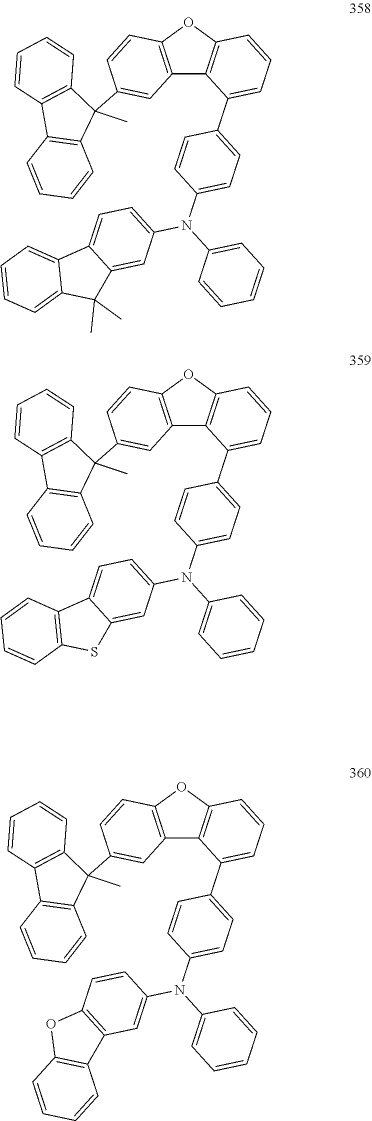 [SMILES: CC1(C)c2ccccc2-c2ccc(N(c3ccccc3)c3ccc(-c4cccc5oc6ccc(C7(C)c8ccccc8-c8ccccc87)cc6c45)cc3)cc21 ; CC1(c2ccc3oc4cccc(-c5ccc(N(c6ccccc6)c6ccc7c(c6)sc6ccccc67)cc5)c4c3c2)c2ccccc2-c2ccccc21 ; CC1(c2ccc3oc4cccc(-c5ccc(N(c6ccccc6)c6ccc7oc8ccccc8c7c6)cc5)c4c3c2)c2ccccc2-c2ccccc21]

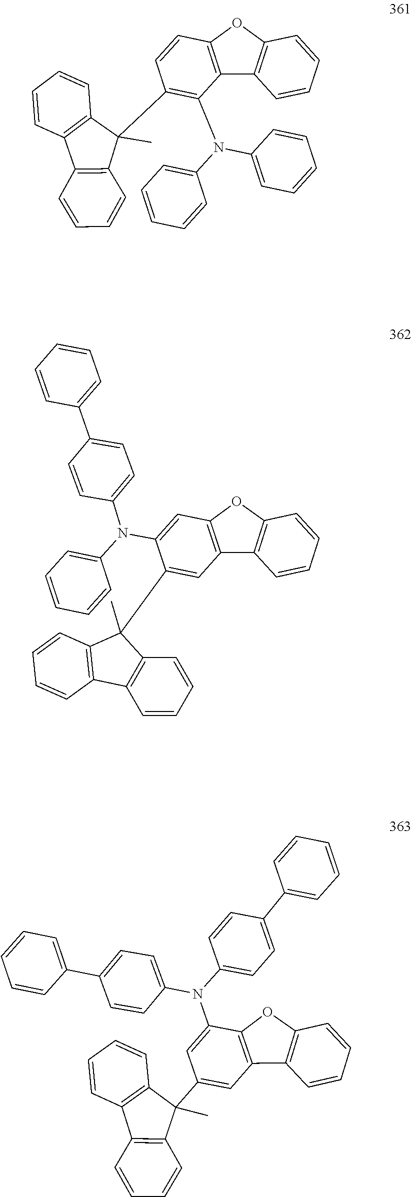 [SMILES: CC1(c2ccc3oc4ccccc4c3c2N(c2ccccc2)c2ccccc2)c2ccccc2-c2ccccc21 ; CC1(c2cc3c(cc2N(c2ccccc2)c2ccc(-c4ccccc4)cc2)oc2ccccc23)c2ccccc2-c2ccccc21 ; CC1(c2cc(N(c3ccc(-c4ccccc4)cc3)c3ccc(-c4ccccc4)cc3)c3oc4ccccc4c3c2)c2ccccc2-c2ccccc21]

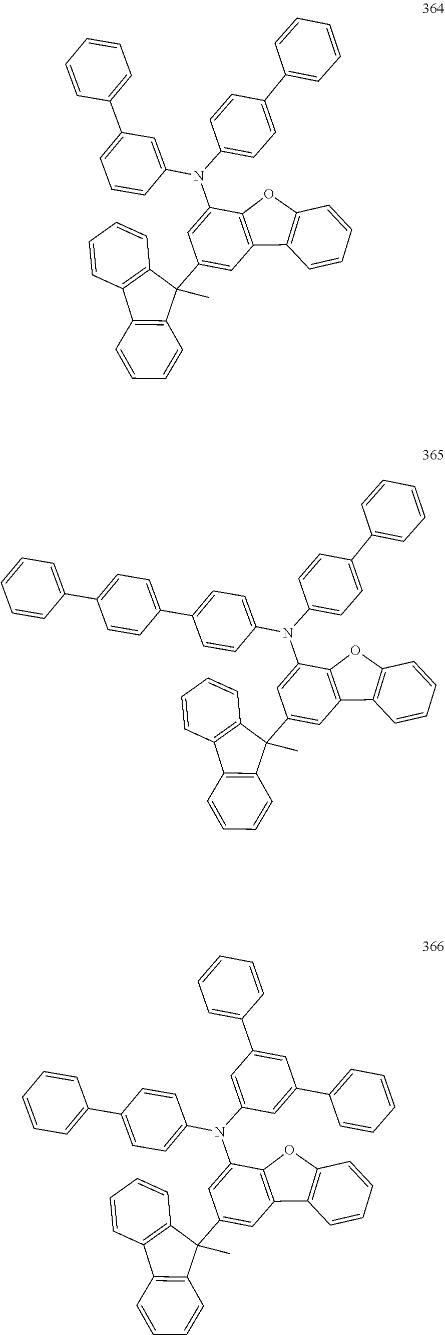 [SMILES: CC1(c2cc(N(c3ccc(-c4ccccc4)cc3)c3cccc(-c4ccccc4)c3)c3oc4ccccc4c3c2)c2ccccc2-c2ccccc21 ; CC1(c2cc(N(c3ccc(-c4ccccc4)cc3)c3ccc(-c4ccc(-c5ccccc5)cc4)cc3)c3oc4ccccc4c3c2)c2ccccc2-c2ccccc21 ; CC1(c2cc(N(c3ccc(-c4ccccc4)cc3)c3cc(-c4ccccc4)cc(-c4ccccc4)c3)c3oc4ccccc4c3c2)c2ccccc2-c2ccccc21]

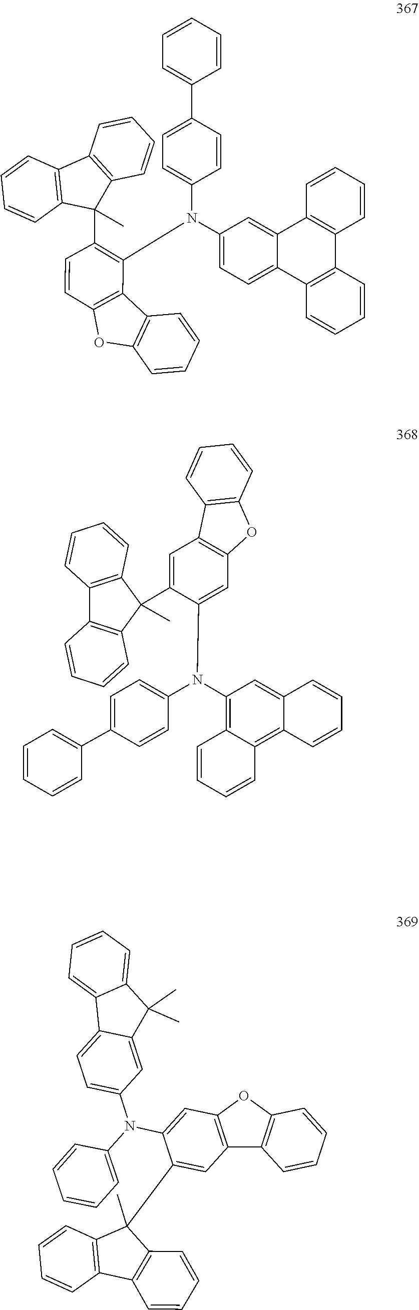 [SMILES: CC1(c2ccc3oc4ccccc4c3c2N(c2ccc(-c3ccccc3)cc2)c2ccc3c4ccccc4c4ccccc4c3c2)c2ccccc2-c2ccccc21 ; CC1(c2cc3c(cc2N(c2ccc(-c4ccccc4)cc2)c2cc4ccccc4c4ccccc24)oc2ccccc23)c2ccccc2-c2ccccc21 ; CC1(C)c2ccccc2-c2ccc(N(c3ccccc3)c3cc4oc5ccccc5c4cc3C3(C)c4ccccc4-c4ccccc43)cc21]

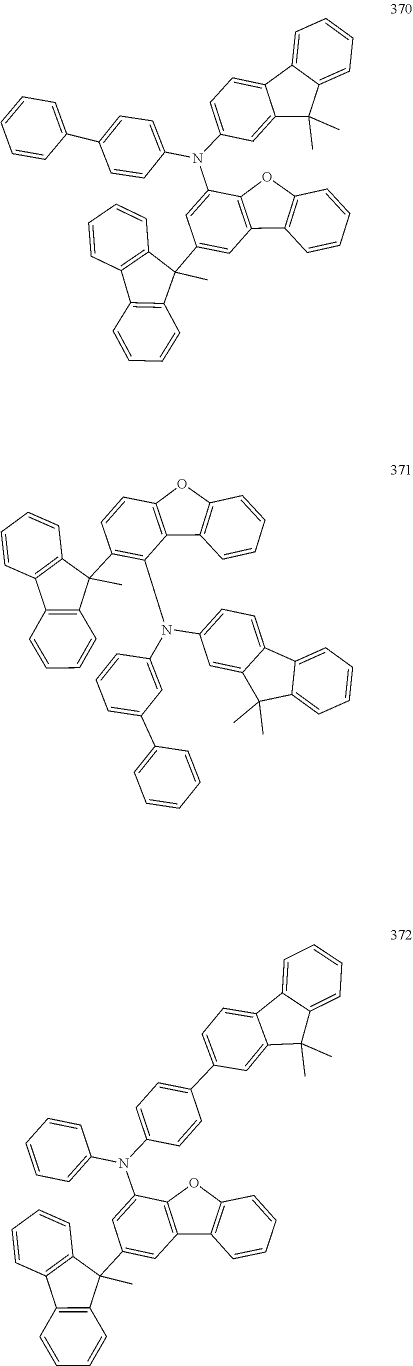 [SMILES: CC1(C)c2ccccc2-c2ccc(N(c3ccc(-c4ccccc4)cc3)c3cc(C4(C)c5ccccc5-c5ccccc54)cc4c3oc3ccccc34)cc21 ; CC1(C)c2ccccc2-c2ccc(N(c3cccc(-c4ccccc4)c3)c3c(C4(C)c5ccccc5-c5ccccc54)ccc4oc5ccccc5c34)cc21 ; CC1(C)c2ccccc2-c2ccc(-c3ccc(N(c4ccccc4)c4cc(C5(C)c6ccccc6-c6ccccc65)cc5c4oc4ccccc45)cc3)cc21]

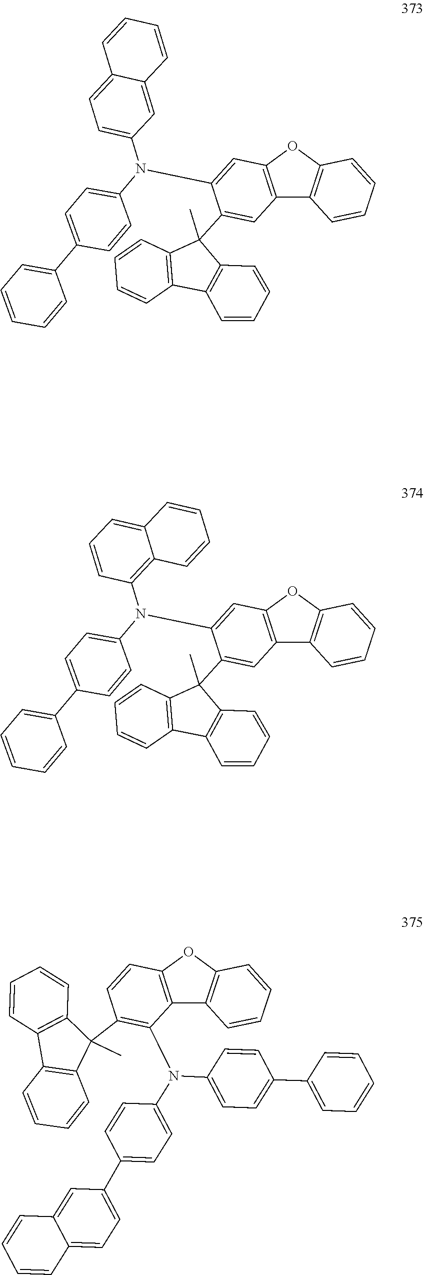 [SMILES: CC1(c2cc3c(cc2N(c2ccc(-c4ccccc4)cc2)c2ccc4ccccc4c2)oc2ccccc23)c2ccccc2-c2ccccc21 ; CC1(c2cc3c(cc2N(c2ccc(-c4ccccc4)cc2)c2cccc4ccccc24)oc2ccccc23)c2ccccc2-c2ccccc21 ; CC1(c2ccc3oc4ccccc4c3c2N(c2ccc(-c3ccccc3)cc2)c2ccc(-c3ccc4ccccc4c3)cc2)c2ccccc2-c2ccccc21]

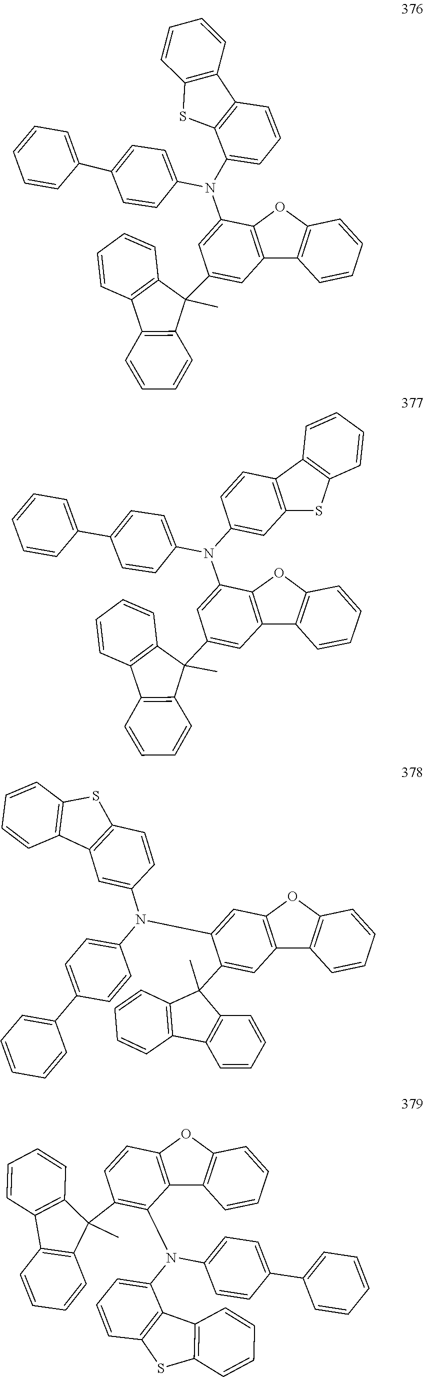 [SMILES: CC1(c2cc(N(c3ccc(-c4ccccc4)cc3)c3cccc4c3sc3ccccc34)c3oc4ccccc4c3c2)c2ccccc2-c2ccccc21 ; CC1(c2cc(N(c3ccc(-c4ccccc4)cc3)c3ccc4c(c3)sc3ccccc34)c3oc4ccccc4c3c2)c2ccccc2-c2ccccc21 ; CC1(c2cc3c(cc2N(c2ccc(-c4ccccc4)cc2)c2ccc4sc5ccccc5c4c2)oc2ccccc23)c2ccccc2-c2ccccc21 ; CC1(c2ccc3oc4ccccc4c3c2N(c2ccc(-c3ccccc3)cc2)c2cccc3sc4ccccc4c23)c2ccccc2-c2ccccc21]

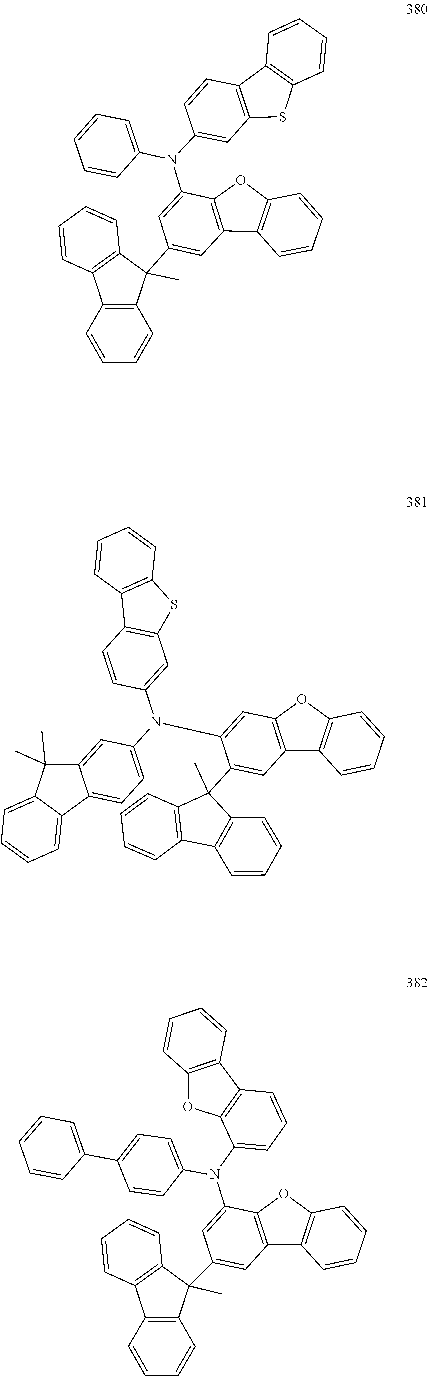 [SMILES: CC1(c2cc(N(c3ccccc3)c3ccc4c(c3)sc3ccccc34)c3oc4ccccc4c3c2)c2ccccc2-c2ccccc21 ; CC1(C)c2ccccc2-c2ccc(N(c3ccc4c(c3)sc3ccccc34)c3cc4oc5ccccc5c4cc3C3(C)c4ccccc4-c4ccccc43)cc21 ; CC1(c2cc(N(c3ccc(-c4ccccc4)cc3)c3cccc4c3oc3ccccc34)c3oc4ccccc4c3c2)c2ccccc2-c2ccccc21]

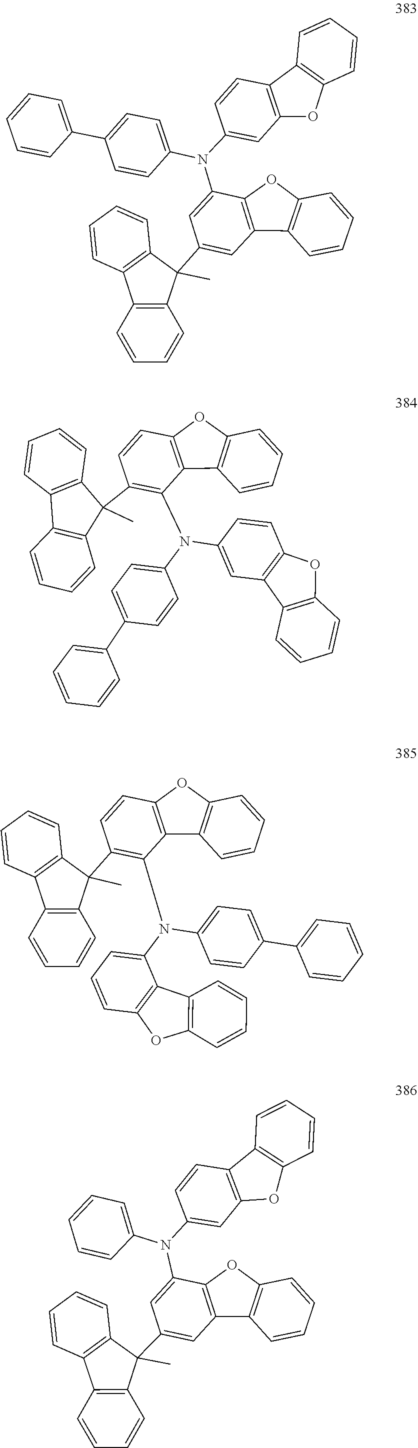 [SMILES: CC1(c2cc(N(c3ccc(-c4ccccc4)cc3)c3ccc4c(c3)oc3ccccc34)c3oc4ccccc4c3c2)c2ccccc2-c2ccccc21 ; CC1(c2ccc3oc4ccccc4c3c2N(c2ccc(-c3ccccc3)cc2)c2ccc3oc4ccccc4c3c2)c2ccccc2-c2ccccc21 ; CC1(c2ccc3oc4ccccc4c3c2N(c2ccc(-c3ccccc3)cc2)c2cccc3oc4ccccc4c23)c2ccccc2-c2ccccc21 ; CC1(c2cc(N(c3ccccc3)c3ccc4c(c3)oc3ccccc34)c3oc4ccccc4c3c2)c2ccccc2-c2ccccc21]

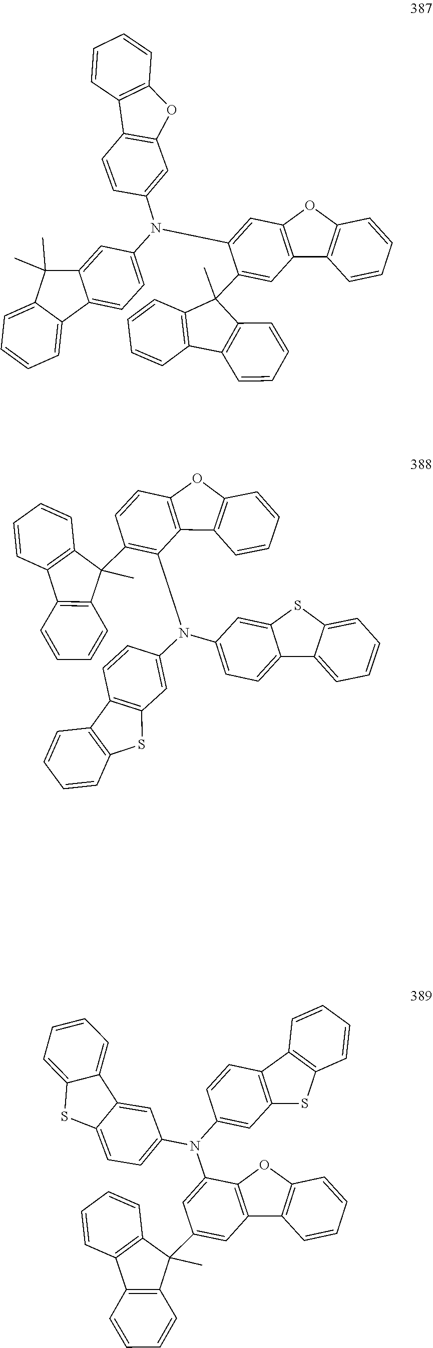 [SMILES: CC1(C)c2ccccc2-c2ccc(N(c3ccc4c(c3)oc3ccccc34)c3cc4oc5ccccc5c4cc3C3(C)c4ccccc4-c4ccccc43)cc21 ; CC1(c2ccc3oc4ccccc4c3c2N(c2ccc3c(c2)sc2ccccc23)c2ccc3c(c2)sc2ccccc23)c2ccccc2-c2ccccc21 ; CC1(c2cc(N(c3ccc4c(c3)sc3ccccc34)c3ccc4sc5ccccc5c4c3)c3oc4ccccc4c3c2)c2ccccc2-c2ccccc21]

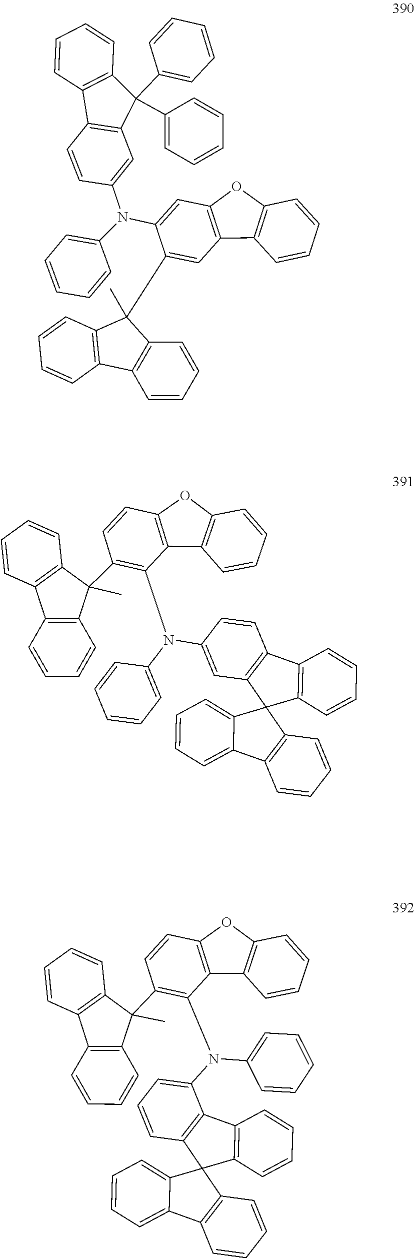 [SMILES: CC1(c2cc3c(cc2N(c2ccccc2)c2ccc4c(c2)C(c2ccccc2)(c2ccccc2)c2ccccc2-4)oc2ccccc23)c2ccccc2-c2ccccc21 ; CC1(c2ccc3oc4ccccc4c3c2N(c2ccccc2)c2ccc3c(c2)C2(c4ccccc4-c4ccccc42)c2ccccc2-3)c2ccccc2-c2ccccc21 ; CC1(c2ccc3oc4ccccc4c3c2N(c2ccccc2)c2cccc3c2-c2ccccc2C32c3ccccc3-c3ccccc32)c2ccccc2-c2ccccc21]

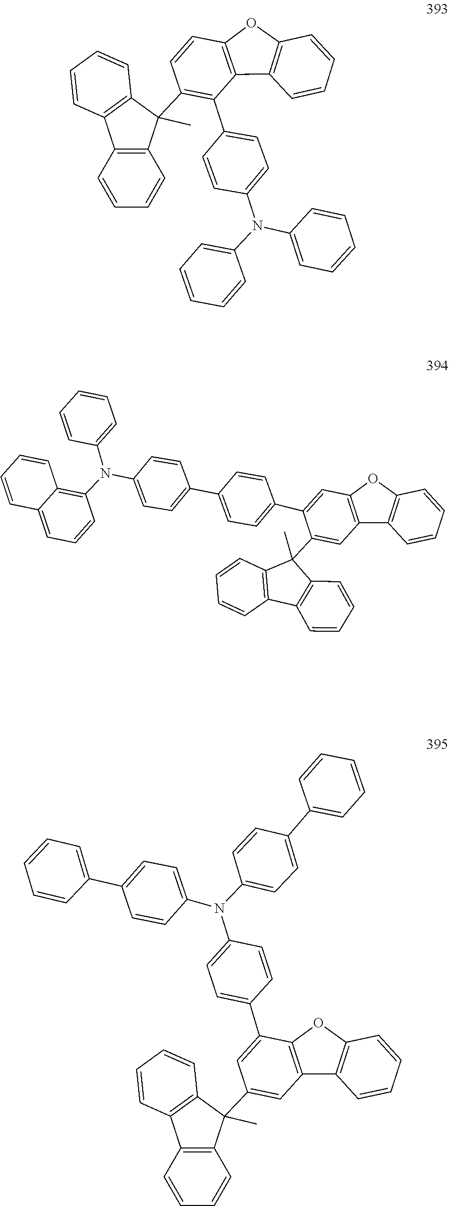 [SMILES: CC1(c2ccc3oc4ccccc4c3c2-c2ccc(N(c3ccccc3)c3ccccc3)cc2)c2ccccc2-c2ccccc21 ; CC1(c2cc3c(cc2-c2ccc(-c4ccc(N(c5ccccc5)c5cccc6ccccc56)cc4)cc2)oc2ccccc23)c2ccccc2-c2ccccc21 ; CC1(c2cc(-c3ccc(N(c4ccc(-c5ccccc5)cc4)c4ccc(-c5ccccc5)cc4)cc3)c3oc4ccccc4c3c2)c2ccccc2-c2ccccc21]

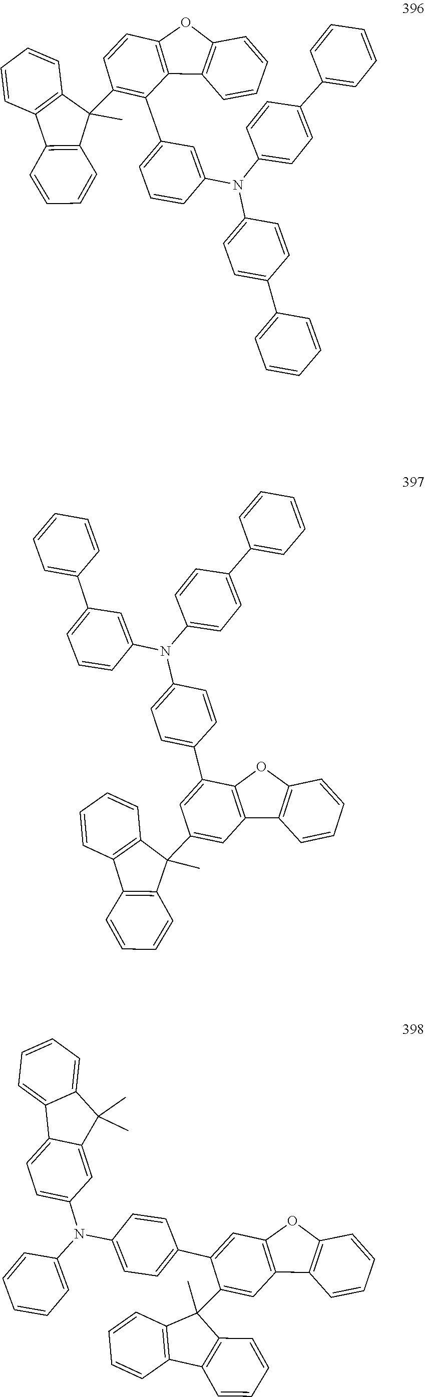 [SMILES: CC1(c2ccc3oc4ccccc4c3c2-c2cccc(N(c3ccc(-c4ccccc4)cc3)c3ccc(-c4ccccc4)cc3)c2)c2ccccc2-c2ccccc21 ; CC1(c2cc(-c3ccc(N(c4ccc(-c5ccccc5)cc4)c4cccc(-c5ccccc5)c4)cc3)c3oc4ccccc4c3c2)c2ccccc2-c2ccccc21 ; CC1(C)c2ccccc2-c2ccc(N(c3ccccc3)c3ccc(-c4cc5oc6ccccc6c5cc4C4(C)c5ccccc5-c5ccccc54)cc3)cc21]

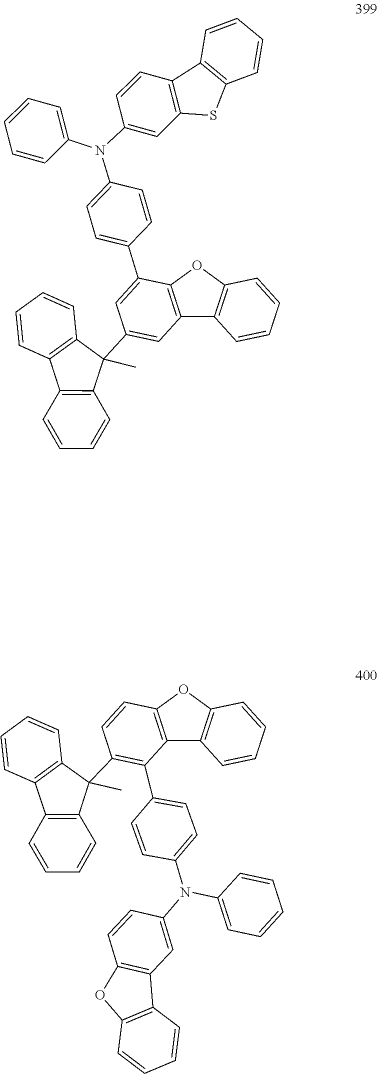 [SMILES: CC1(c2cc(-c3ccc(N(c4ccccc4)c4ccc5c(c4)sc4ccccc45)cc3)c3oc4ccccc4c3c2)c2ccccc2-c2ccccc21 ; CC1(c2ccc3oc4ccccc4c3c2-c2ccc(N(c3ccccc3)c3ccc4oc5ccccc5c4c3)cc2)c2ccccc2-c2ccccc21]

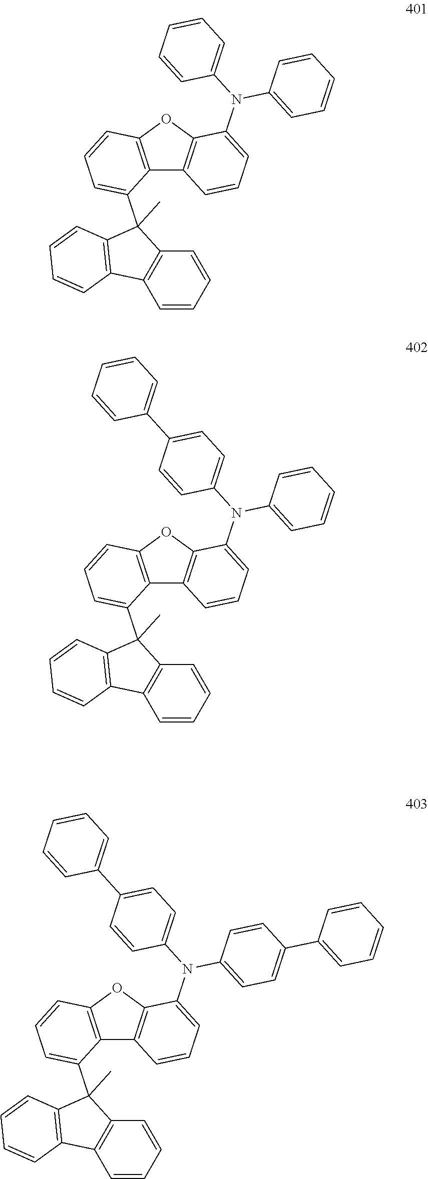 [SMILES: CC1(c2cccc3oc4c(N(c5ccccc5)c5ccccc5)cccc4c23)c2ccccc2-c2ccccc21 ; CC1(c2cccc3oc4c(N(c5ccccc5)c5ccc(-c6ccccc6)cc5)cccc4c23)c2ccccc2-c2ccccc21 ; CC1(c2cccc3oc4c(N(c5ccc(-c6ccccc6)cc5)c5ccc(-c6ccccc6)cc5)cccc4c23)c2ccccc2-c2ccccc21]

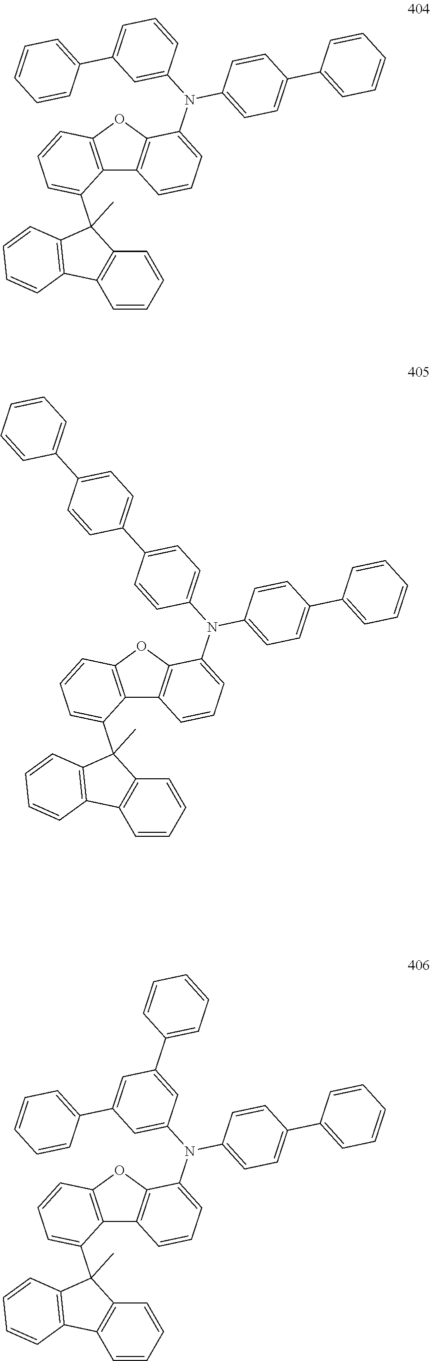 [SMILES: CC1(c2cccc3oc4c(N(c5ccc(-c6ccccc6)cc5)c5cccc(-c6ccccc6)c5)cccc4c23)c2ccccc2-c2ccccc21 ; CC1(c2cccc3oc4c(N(c5ccc(-c6ccccc6)cc5)c5ccc(-c6ccc(-c7ccccc7)cc6)cc5)cccc4c23)c2ccccc2-c2ccccc21 ; CC1(c2cccc3oc4c(N(c5ccc(-c6ccccc6)cc5)c5cc(-c6ccccc6)cc(-c6ccccc6)c5)cccc4c23)c2ccccc2-c2ccccc21]

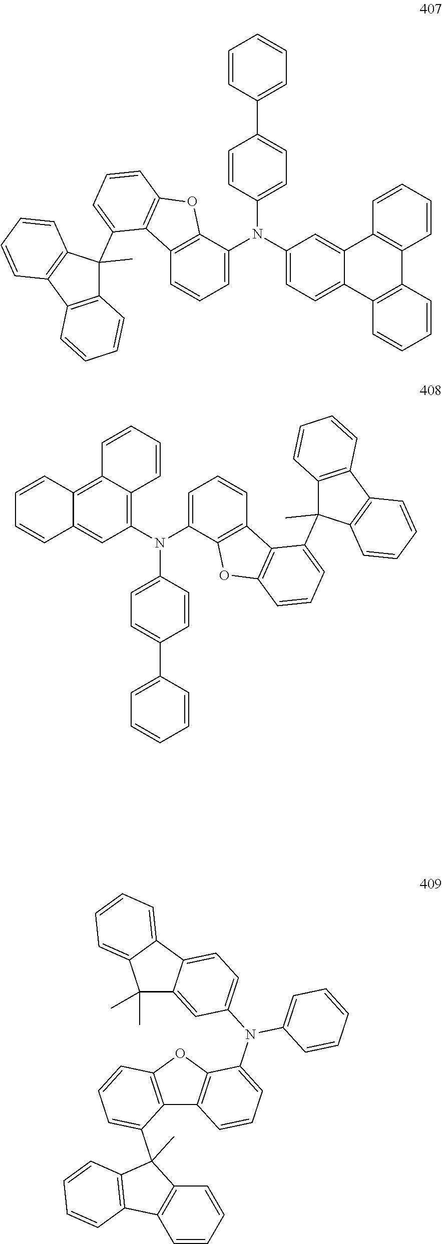 [SMILES: CC1(c2cccc3oc4c(N(c5ccc(-c6ccccc6)cc5)c5ccc6c7ccccc7c7ccccc7c6c5)cccc4c23)c2ccccc2-c2ccccc21 ; CC1(c2cccc3oc4c(N(c5ccc(-c6ccccc6)cc5)c5cc6ccccc6c6ccccc56)cccc4c23)c2ccccc2-c2ccccc21 ; CC1(C)c2ccccc2-c2ccc(N(c3ccccc3)c3cccc4c3oc3cccc(C5(C)c6ccccc6-c6ccccc65)c34)cc21]

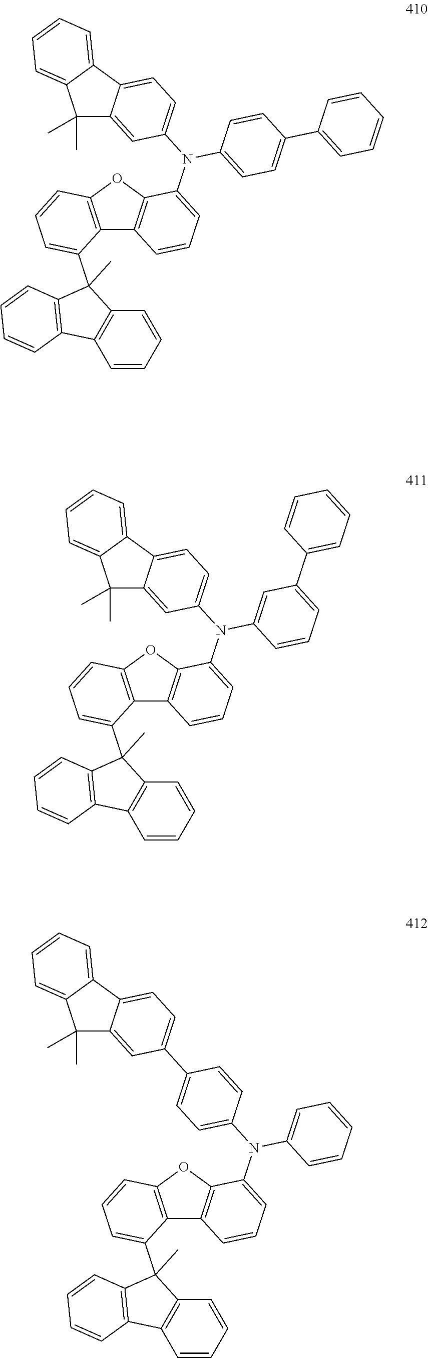 [SMILES: CC1(C)c2ccccc2-c2ccc(N(c3ccc(-c4ccccc4)cc3)c3cccc4c3oc3cccc(C5(C)c6ccccc6-c6ccccc65)c34)cc21 ; CC1(C)c2ccccc2-c2ccc(N(c3cccc(-c4ccccc4)c3)c3cccc4c3oc3cccc(C5(C)c6ccccc6-c6ccccc65)c34)cc21 ; CC1(C)c2ccccc2-c2ccc(-c3ccc(N(c4ccccc4)c4cccc5c4oc4cccc(C6(C)c7ccccc7-c7ccccc76)c45)cc3)cc21]

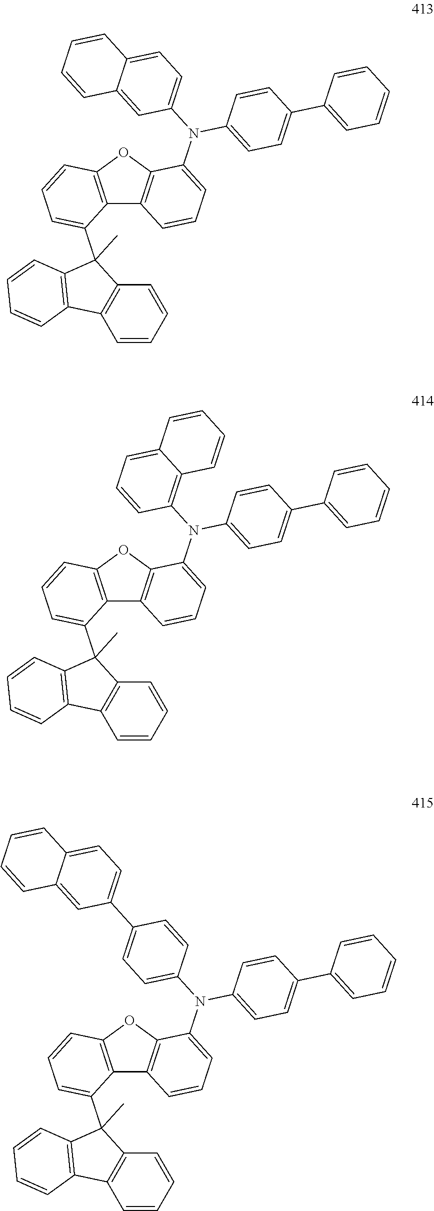 [SMILES: CC1(c2cccc3oc4c(N(c5ccc(-c6ccccc6)cc5)c5ccc6ccccc6c5)cccc4c23)c2ccccc2-c2ccccc21 ; CC1(c2cccc3oc4c(N(c5ccc(-c6ccccc6)cc5)c5cccc6ccccc56)cccc4c23)c2ccccc2-c2ccccc21 ; CC1(c2cccc3oc4c(N(c5ccc(-c6ccccc6)cc5)c5ccc(-c6ccc7ccccc7c6)cc5)cccc4c23)c2ccccc2-c2ccccc21]

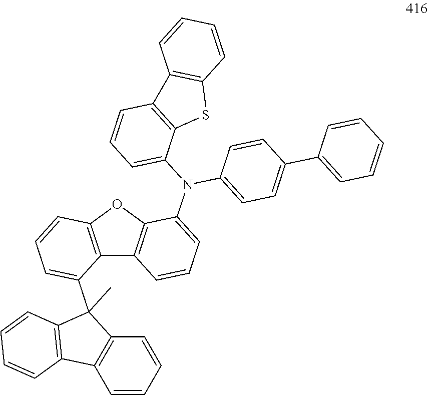 [SMILES: CC1(c2cccc3oc4c(N(c5ccc(-c6ccccc6)cc5)c5cccc6c5sc5ccccc56)cccc4c23)c2ccccc2-c2ccccc21]

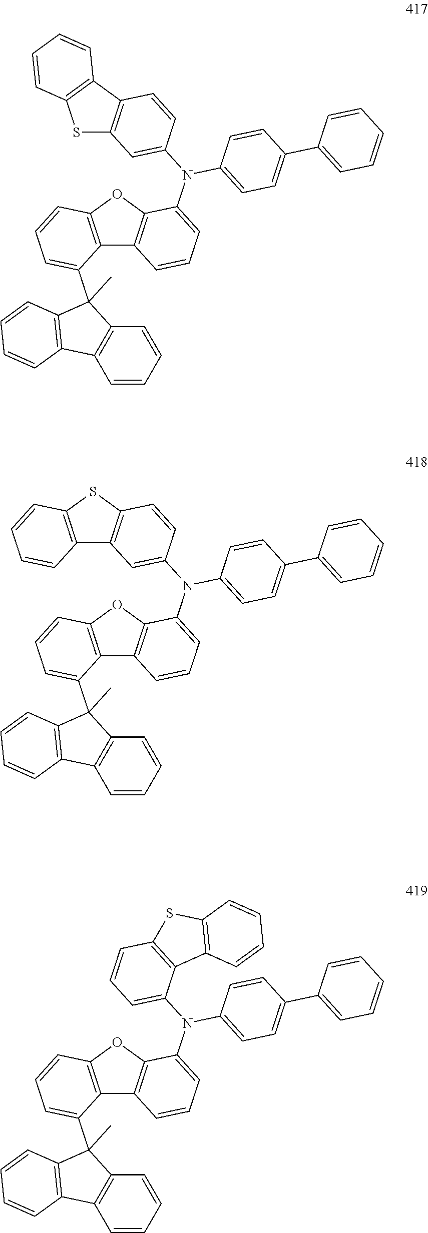 [SMILES: CC1(c2cccc3oc4c(N(c5ccc(-c6ccccc6)cc5)c5ccc6c(c5)sc5ccccc56)cccc4c23)c2ccccc2-c2ccccc21 ; CC1(c2cccc3oc4c(N(c5ccc(-c6ccccc6)cc5)c5ccc6sc7ccccc7c6c5)cccc4c23)c2ccccc2-c2ccccc21 ; CC1(c2cccc3oc4c(N(c5ccc(-c6ccccc6)cc5)c5cccc6sc7ccccc7c56)cccc4c23)c2ccccc2-c2ccccc21]

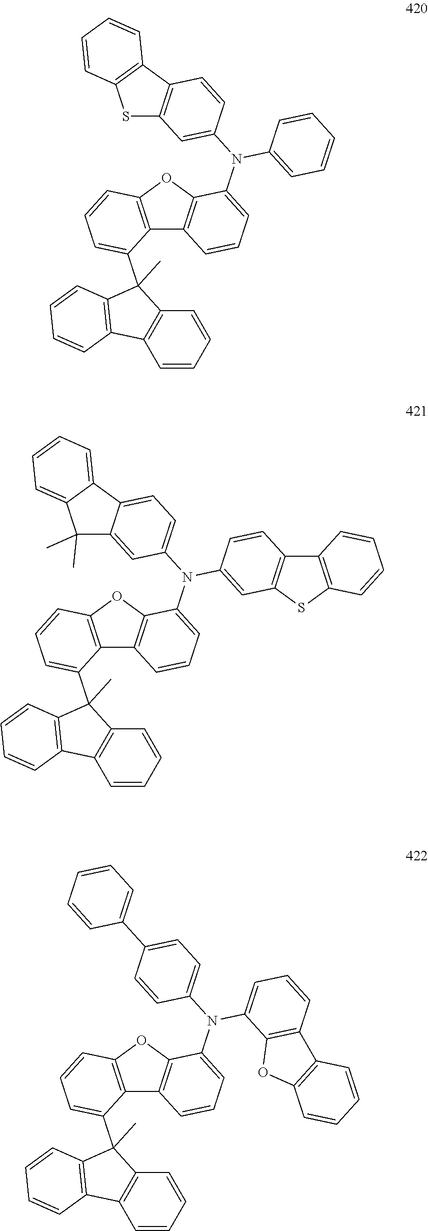 [SMILES: CC1(c2cccc3oc4c(N(c5ccccc5)c5ccc6c(c5)sc5ccccc56)cccc4c23)c2ccccc2-c2ccccc21 ; CC1(C)c2ccccc2-c2ccc(N(c3ccc4c(c3)sc3ccccc34)c3cccc4c3oc3cccc(C5(C)c6ccccc6-c6ccccc65)c34)cc21 ; CC1(c2cccc3oc4c(N(c5ccc(-c6ccccc6)cc5)c5cccc6c5oc5ccccc56)cccc4c23)c2ccccc2-c2ccccc21]

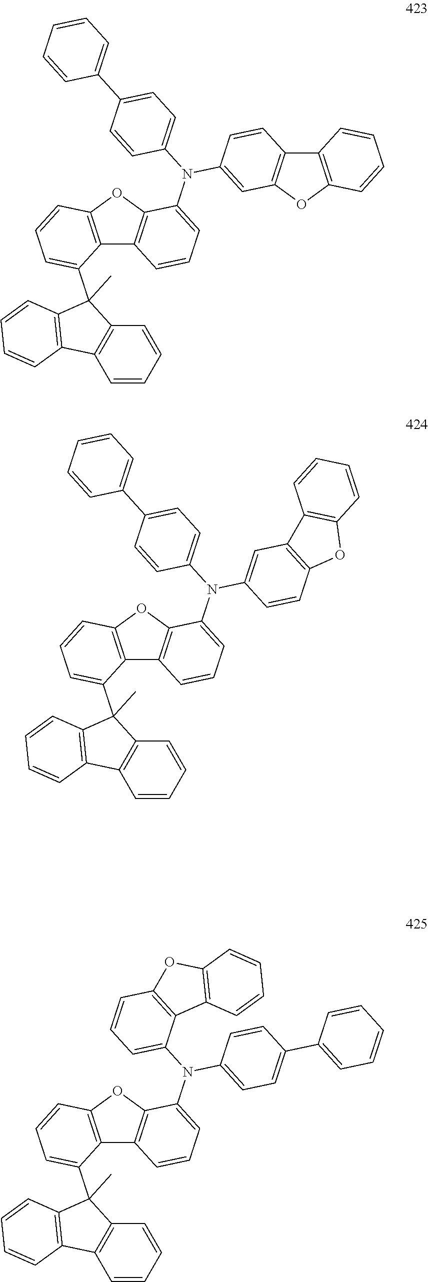 [SMILES: CC1(c2cccc3oc4c(N(c5ccc(-c6ccccc6)cc5)c5ccc6c(c5)oc5ccccc56)cccc4c23)c2ccccc2-c2ccccc21 ; CC1(c2cccc3oc4c(N(c5ccc(-c6ccccc6)cc5)c5ccc6oc7ccccc7c6c5)cccc4c23)c2ccccc2-c2ccccc21 ; CC1(c2cccc3oc4c(N(c5ccc(-c6ccccc6)cc5)c5cccc6oc7ccccc7c56)cccc4c23)c2ccccc2-c2ccccc21]

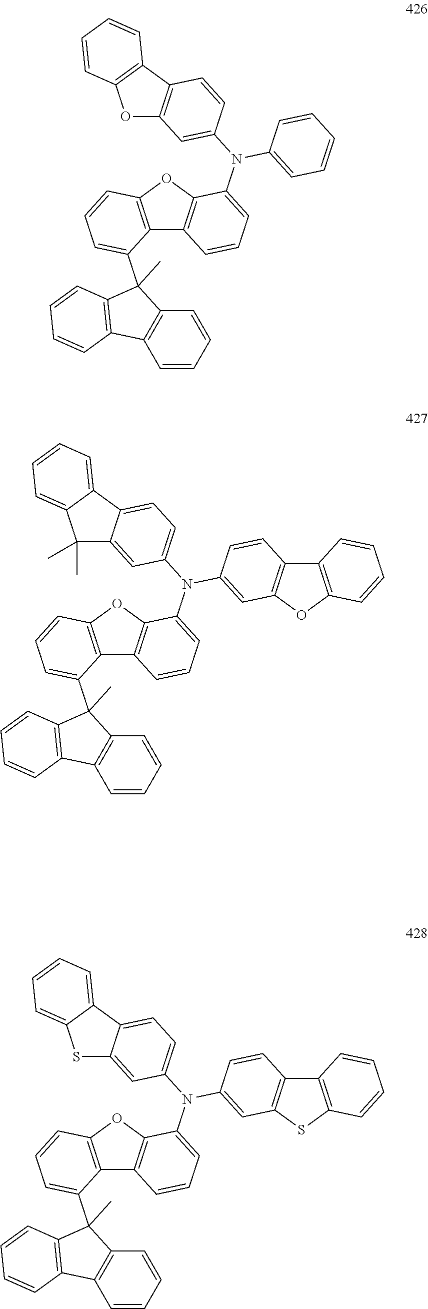 [SMILES: CC1(c2cccc3oc4c(N(c5ccccc5)c5ccc6c(c5)oc5ccccc56)cccc4c23)c2ccccc2-c2ccccc21 ; CC1(C)c2ccccc2-c2ccc(N(c3ccc4c(c3)oc3ccccc34)c3cccc4c3oc3cccc(C5(C)c6ccccc6-c6ccccc65)c34)cc21 ; CC1(c2cccc3oc4c(N(c5ccc6c(c5)sc5ccccc56)c5ccc6c(c5)sc5ccccc56)cccc4c23)c2ccccc2-c2ccccc21]

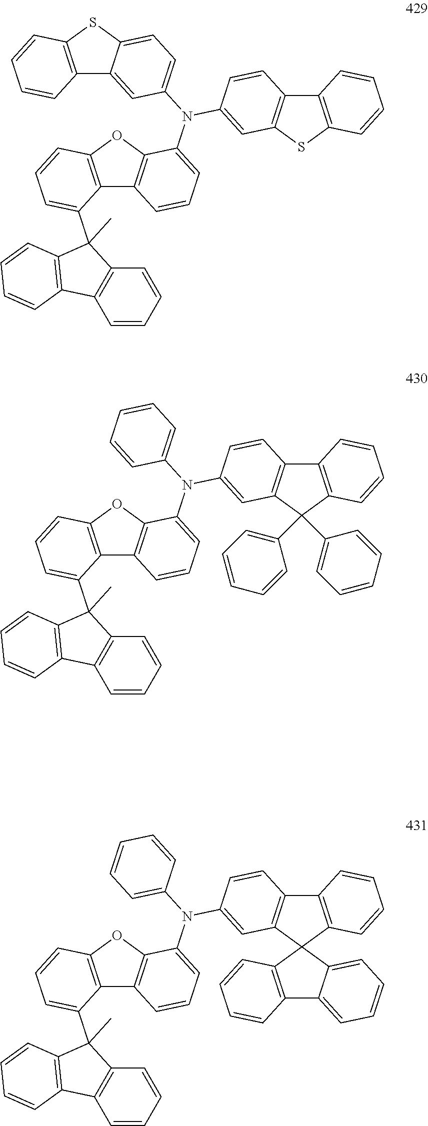 [SMILES: CC1(c2cccc3oc4c(N(c5ccc6c(c5)sc5ccccc56)c5ccc6sc7ccccc7c6c5)cccc4c23)c2ccccc2-c2ccccc21 ; CC1(c2cccc3oc4c(N(c5ccccc5)c5ccc6c(c5)C(c5ccccc5)(c5ccccc5)c5ccccc5-6)cccc4c23)c2ccccc2-c2ccccc21 ; CC1(c2cccc3oc4c(N(c5ccccc5)c5ccc6c(c5)C5(c7ccccc7-c7ccccc75)c5ccccc5-6)cccc4c23)c2ccccc2-c2ccccc21]

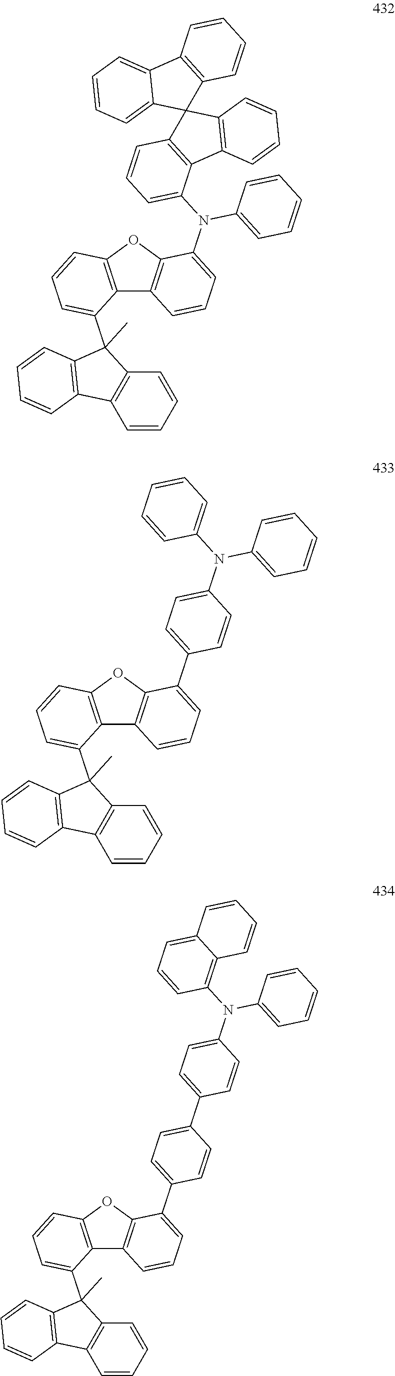 [SMILES: CC1(c2cccc3oc4c(N(c5ccccc5)c5cccc6c5-c5ccccc5C65c6ccccc6-c6ccccc65)cccc4c23)c2ccccc2-c2ccccc21 ; CC1(c2cccc3oc4c(-c5ccc(N(c6ccccc6)c6ccccc6)cc5)cccc4c23)c2ccccc2-c2ccccc21 ; CC1(c2cccc3oc4c(-c5ccc(-c6ccc(N(c7ccccc7)c7cccc8ccccc78)cc6)cc5)cccc4c23)c2ccccc2-c2ccccc21]

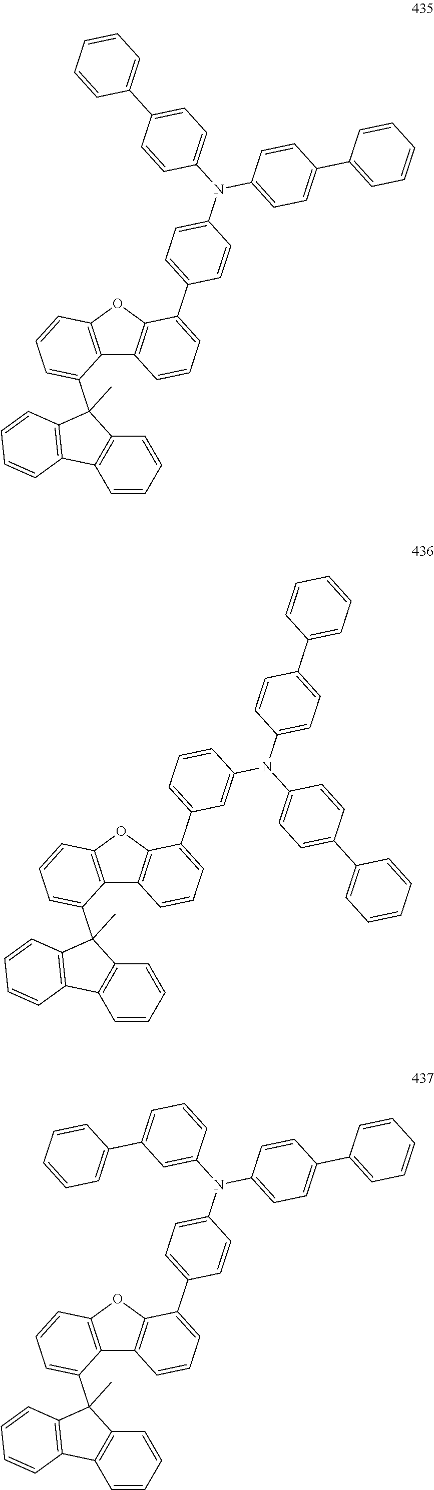 [SMILES: CC1(c2cccc3oc4c(-c5ccc(N(c6ccc(-c7ccccc7)cc6)c6ccc(-c7ccccc7)cc6)cc5)cccc4c23)c2ccccc2-c2ccccc21 ; CC1(c2cccc3oc4c(-c5cccc(N(c6ccc(-c7ccccc7)cc6)c6ccc(-c7ccccc7)cc6)c5)cccc4c23)c2ccccc2-c2ccccc21 ; CC1(c2cccc3oc4c(-c5ccc(N(c6ccc(-c7ccccc7)cc6)c6cccc(-c7ccccc7)c6)cc5)cccc4c23)c2ccccc2-c2ccccc21]

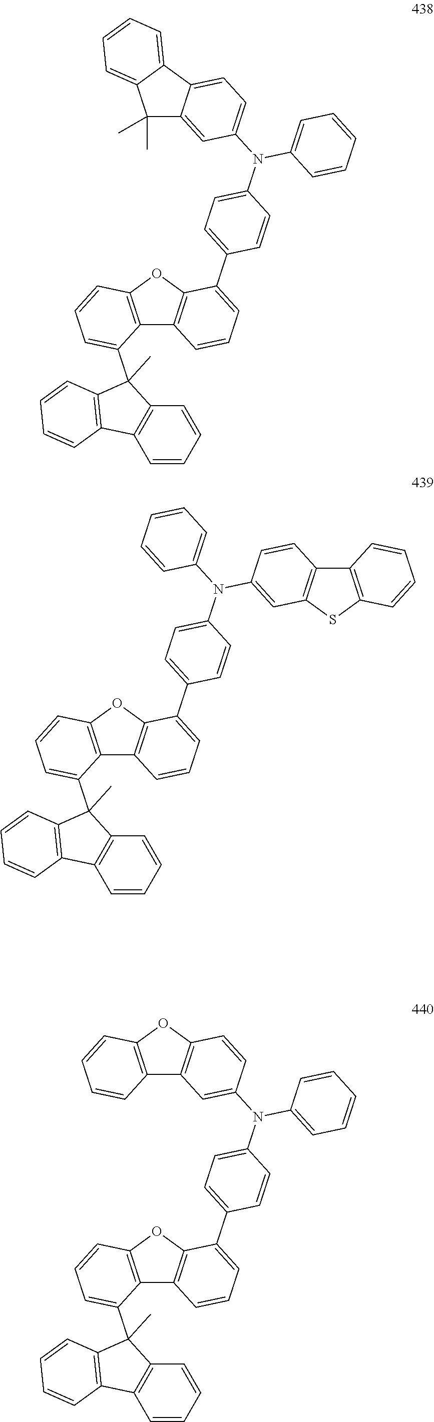

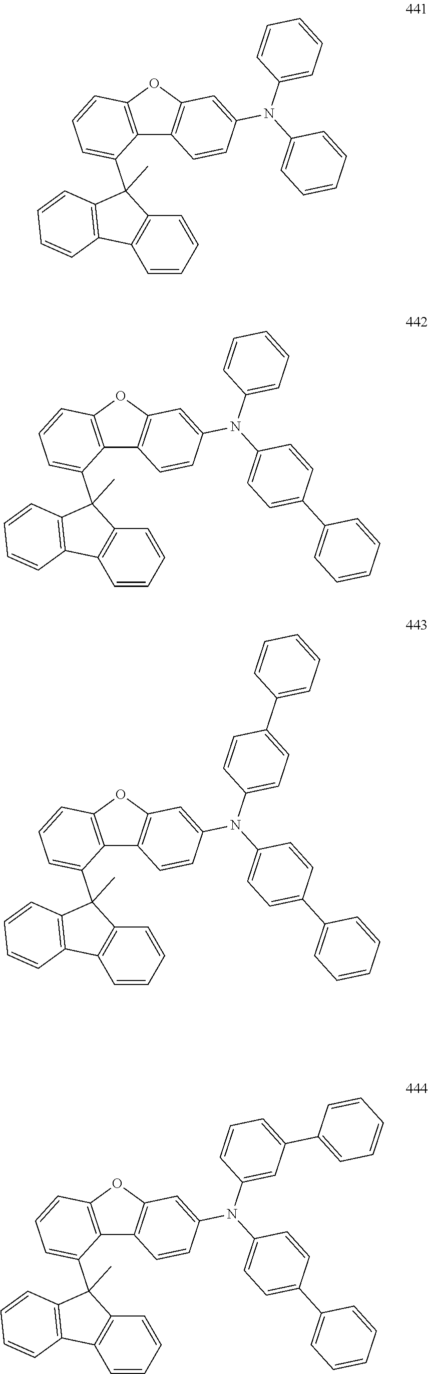 [SMILES: CC1(c2cccc3oc4cc(N(c5ccccc5)c5ccccc5)ccc4c23)c2ccccc2-c2ccccc21 ; CC1(c2cccc3oc4cc(N(c5ccccc5)c5ccc(-c6ccccc6)cc5)ccc4c23)c2ccccc2-c2ccccc21 ; CC1(c2cccc3oc4cc(N(c5ccc(-c6ccccc6)cc5)c5ccc(-c6ccccc6)cc5)ccc4c23)c2ccccc2-c2ccccc21 ; CC1(c2cccc3oc4cc(N(c5ccc(-c6ccccc6)cc5)c5cccc(-c6ccccc6)c5)ccc4c23)c2ccccc2-c2ccccc21]

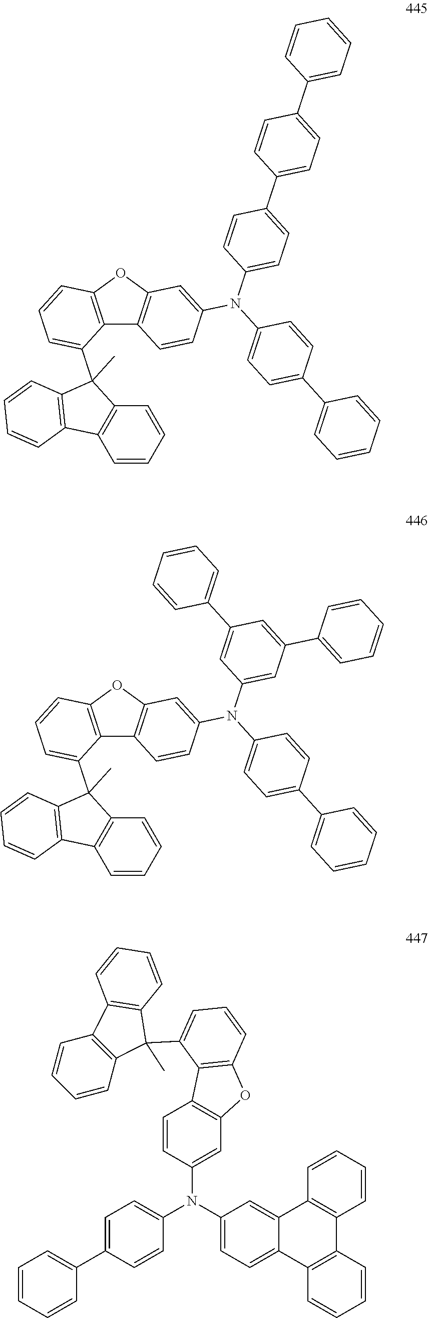 [SMILES: CC1(c2cccc3oc4cc(N(c5ccc(-c6ccccc6)cc5)c5ccc(-c6ccc(-c7ccccc7)cc6)cc5)ccc4c23)c2ccccc2-c2ccccc21 ; CC1(c2cccc3oc4cc(N(c5ccc(-c6ccccc6)cc5)c5cc(-c6ccccc6)cc(-c6ccccc6)c5)ccc4c23)c2ccccc2-c2ccccc21 ; CC1(c2cccc3oc4cc(N(c5ccc(-c6ccccc6)cc5)c5ccc6c7ccccc7c7ccccc7c6c5)ccc4c23)c2ccccc2-c2ccccc21]

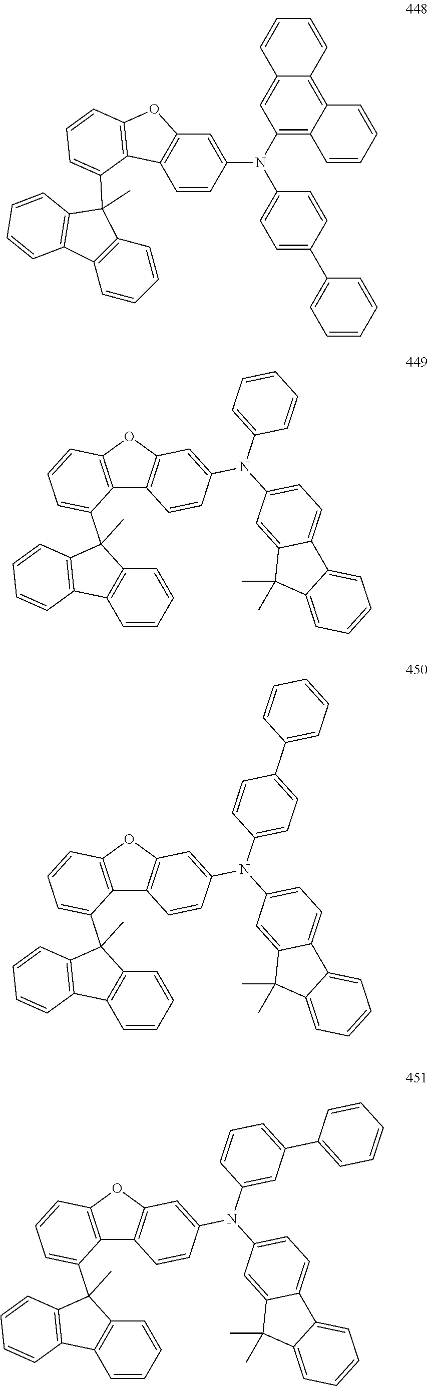 [SMILES: CC1(c2cccc3oc4cc(N(c5ccc(-c6ccccc6)cc5)c5cc6ccccc6c6ccccc56)ccc4c23)c2ccccc2-c2ccccc21 ; CC1(C)c2ccccc2-c2ccc(N(c3ccccc3)c3ccc4c(c3)oc3cccc(C5(C)c6ccccc6-c6ccccc65)c34)cc21 ; CC1(C)c2ccccc2-c2ccc(N(c3ccc(-c4ccccc4)cc3)c3ccc4c(c3)oc3cccc(C5(C)c6ccccc6-c6ccccc65)c34)cc21 ; CC1(C)c2ccccc2-c2ccc(N(c3cccc(-c4ccccc4)c3)c3ccc4c(c3)oc3cccc(C5(C)c6ccccc6-c6ccccc65)c34)cc21]

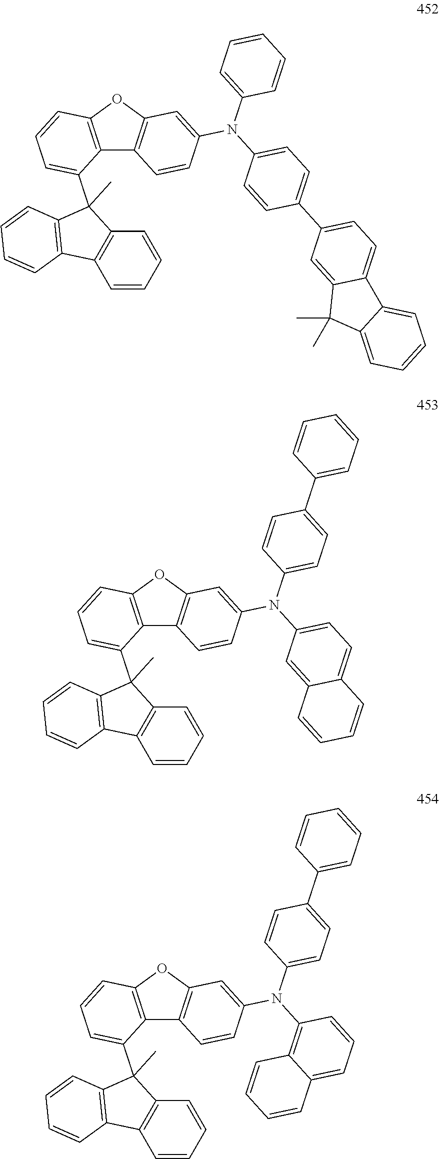 [SMILES: CC1(C)c2ccccc2-c2ccc(-c3ccc(N(c4ccccc4)c4ccc5c(c4)oc4cccc(C6(C)c7ccccc7-c7ccccc76)c45)cc3)cc21 ; CC1(c2cccc3oc4cc(N(c5ccc(-c6ccccc6)cc5)c5ccc6ccccc6c5)ccc4c23)c2ccccc2-c2ccccc21 ; CC1(c2cccc3oc4cc(N(c5ccc(-c6ccccc6)cc5)c5cccc6ccccc56)ccc4c23)c2ccccc2-c2ccccc21]

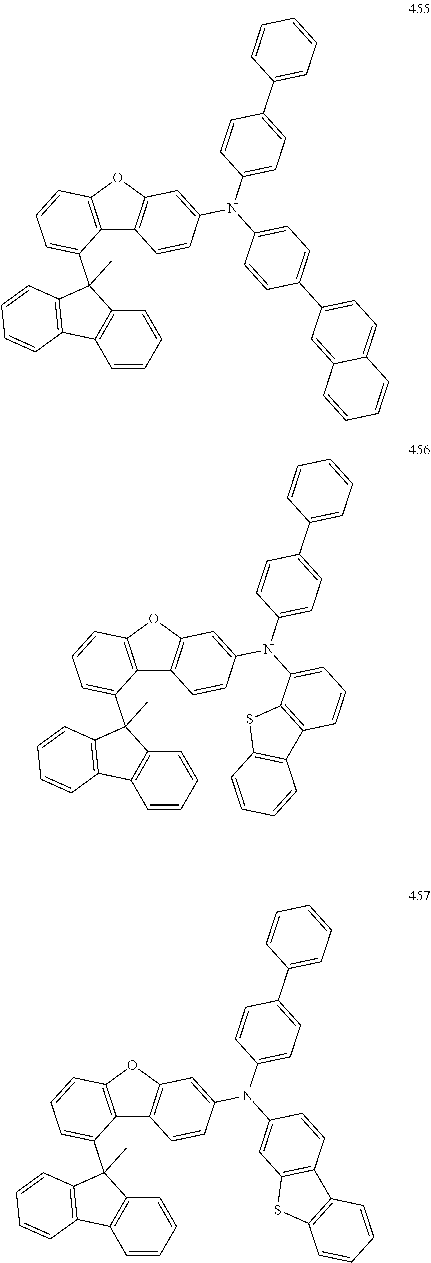

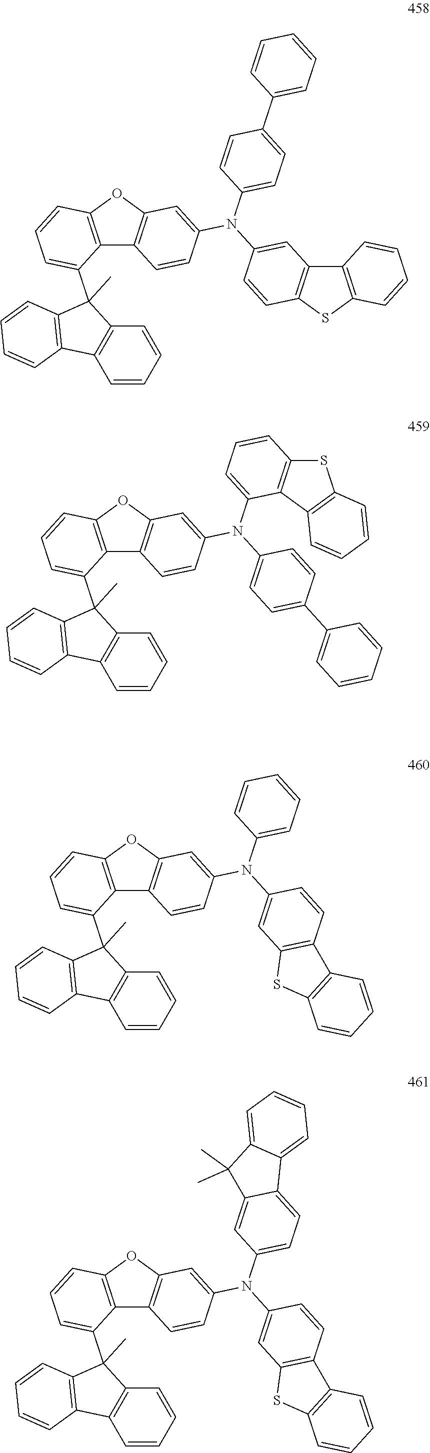 [SMILES: CC1(c2cccc3oc4cc(N(c5ccc(-c6ccccc6)cc5)c5ccc6sc7ccccc7c6c5)ccc4c23)c2ccccc2-c2ccccc21 ; CC1(c2cccc3oc4cc(N(c5ccc(-c6ccccc6)cc5)c5cccc6sc7ccccc7c56)ccc4c23)c2ccccc2-c2ccccc21 ; CC1(c2cccc3oc4cc(N(c5ccccc5)c5ccc6c(c5)sc5ccccc56)ccc4c23)c2ccccc2-c2ccccc21 ; CC1(C)c2ccccc2-c2ccc(N(c3ccc4c(c3)oc3cccc(C5(C)c6ccccc6-c6ccccc65)c34)c3ccc4c(c3)sc3ccccc34)cc21]

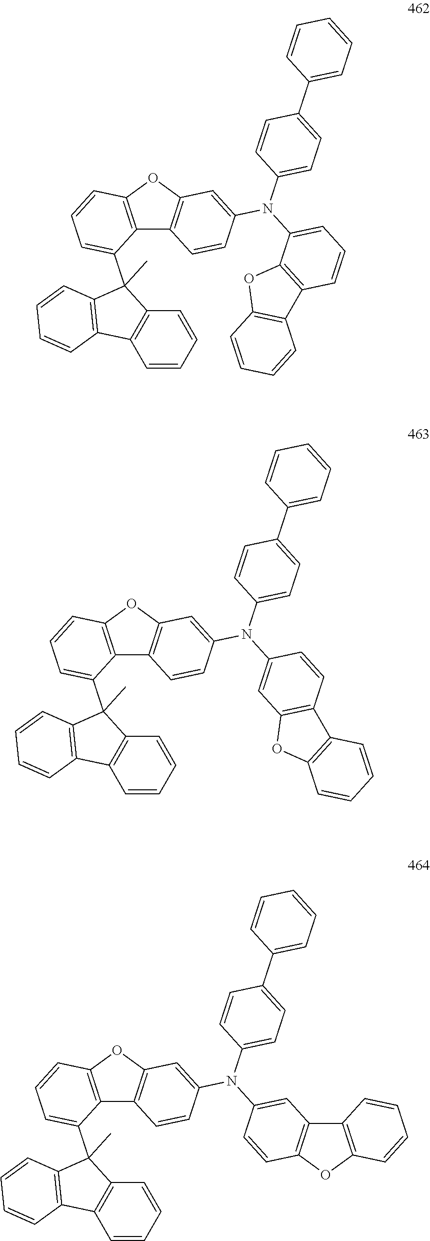 [SMILES: CC1(c2cccc3oc4cc(N(c5ccc(-c6ccccc6)cc5)c5cccc6c5oc5ccccc56)ccc4c23)c2ccccc2-c2ccccc21 ; CC1(c2cccc3oc4cc(N(c5ccc(-c6ccccc6)cc5)c5ccc6c(c5)oc5ccccc56)ccc4c23)c2ccccc2-c2ccccc21 ; CC1(c2cccc3oc4cc(N(c5ccc(-c6ccccc6)cc5)c5ccc6oc7ccccc7c6c5)ccc4c23)c2ccccc2-c2ccccc21]

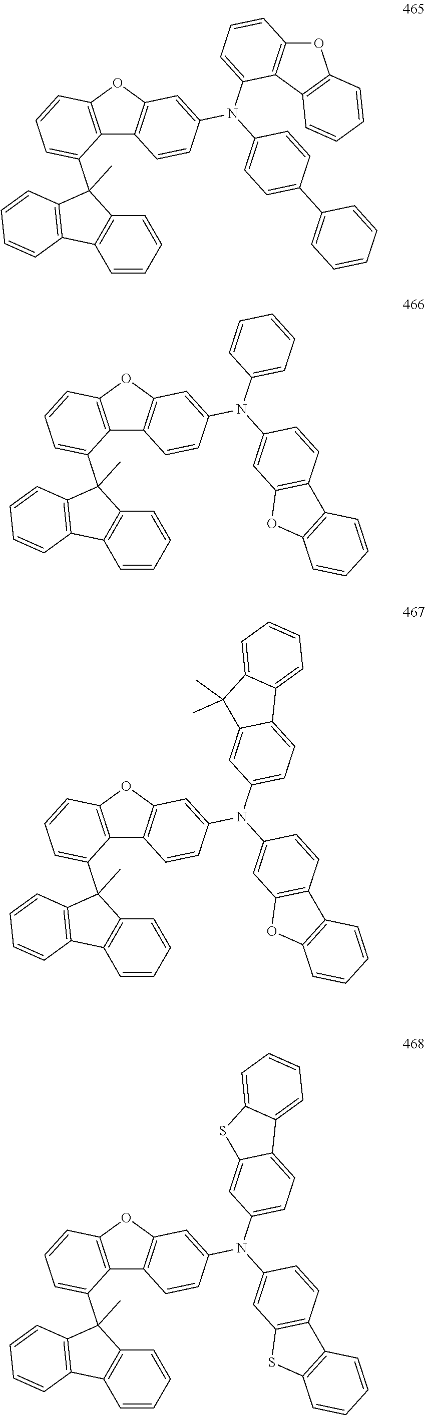 [SMILES: CC1(c2cccc3oc4cc(N(c5ccc(-c6ccccc6)cc5)c5cccc6oc7ccccc7c56)ccc4c23)c2ccccc2-c2ccccc21 ; CC1(c2cccc3oc4cc(N(c5ccccc5)c5ccc6c(c5)oc5ccccc56)ccc4c23)c2ccccc2-c2ccccc21 ; CC1(C)c2ccccc2-c2ccc(N(c3ccc4c(c3)oc3ccccc34)c3ccc4c(c3)oc3cccc(C5(C)c6ccccc6-c6ccccc65)c34)cc21 ; CC1(c2cccc3oc4cc(N(c5ccc6c(c5)sc5ccccc56)c5ccc6c(c5)sc5ccccc56)ccc4c23)c2ccccc2-c2ccccc21]

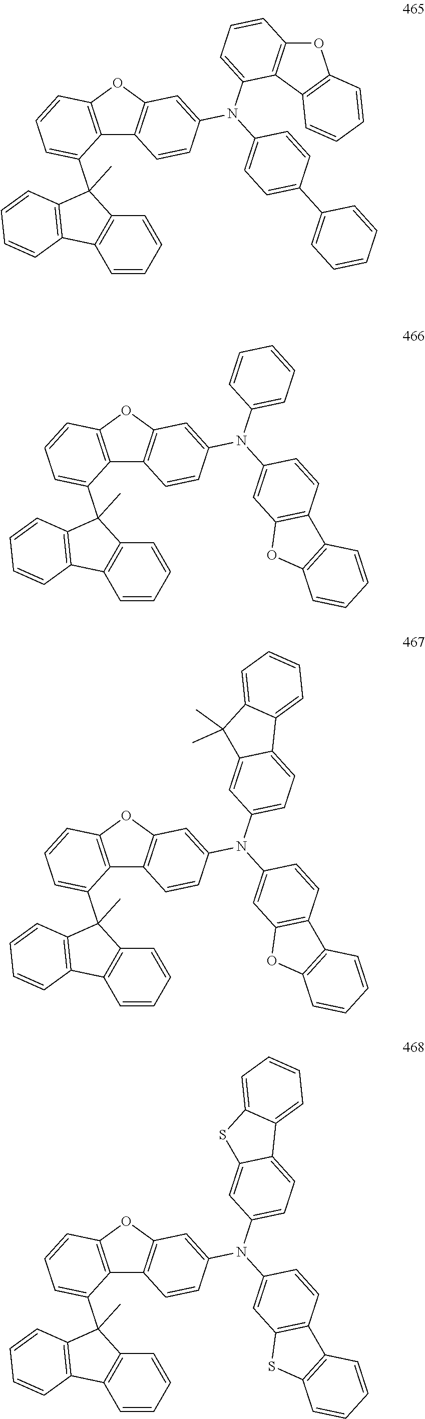 [SMILES: CC1(c2cccc3oc4cc(N(c5ccc(-c6ccccc6)cc5)c5cccc6oc7ccccc7c56)ccc4c23)c2ccccc2-c2ccccc21 ; CC1(c2cccc3oc4cc(N(c5ccccc5)c5ccc6c(c5)oc5ccccc56)ccc4c23)c2ccccc2-c2ccccc21 ; CC1(C)c2ccccc2-c2ccc(N(c3ccc4c(c3)oc3ccccc34)c3ccc4c(c3)oc3cccc(C5(C)c6ccccc6-c6ccccc65)c34)cc21 ; CC1(c2cccc3oc4cc(N(c5ccc6c(c5)sc5ccccc56)c5ccc6c(c5)sc5ccccc56)ccc4c23)c2ccccc2-c2ccccc21]

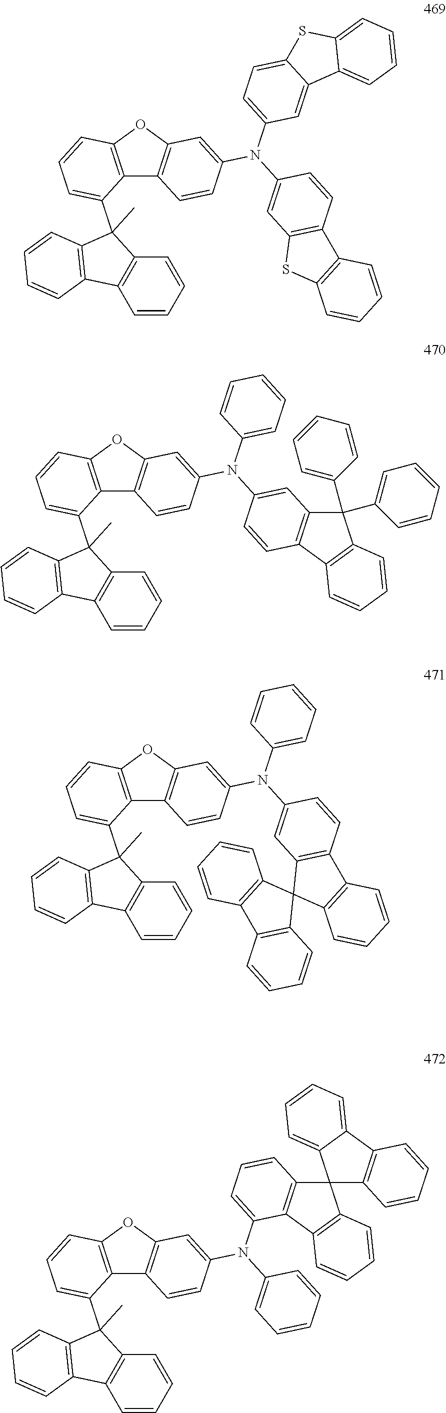 [SMILES: CC1(c2cccc3oc4cc(N(c5ccc6c(c5)sc5ccccc56)c5ccc6sc7ccccc7c6c5)ccc4c23)c2ccccc2-c2ccccc21 ; CC1(c2cccc3oc4cc(N(c5ccccc5)c5ccc6c(c5)C(c5ccccc5)(c5ccccc5)c5ccccc5-6)ccc4c23)c2ccccc2-c2ccccc21 ; CC1(c2cccc3oc4cc(N(c5ccccc5)c5ccc6c(c5)C5(c7ccccc7-c7ccccc75)c5ccccc5-6)ccc4c23)c2ccccc2-c2ccccc21 ; CC1(c2cccc3oc4cc(N(c5ccccc5)c5cccc6c5-c5ccccc5C65c6ccccc6-c6ccccc65)ccc4c23)c2ccccc2-c2ccccc21]

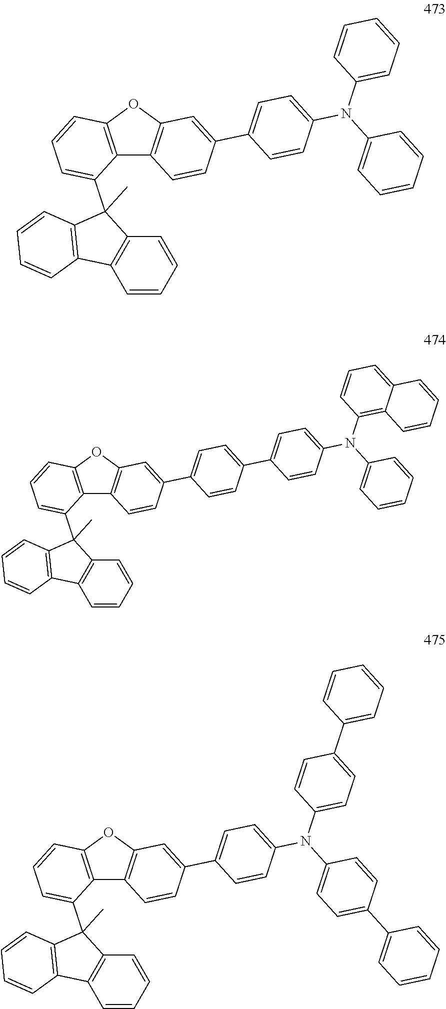 [SMILES: CC1(c2cccc3oc4cc(-c5ccc(N(c6ccccc6)c6ccccc6)cc5)ccc4c23)c2ccccc2-c2ccccc21 ; CC1(c2cccc3oc4cc(-c5ccc(-c6ccc(N(c7ccccc7)c7cccc8ccccc78)cc6)cc5)ccc4c23)c2ccccc2-c2ccccc21 ; CC1(c2cccc3oc4cc(-c5ccc(N(c6ccc(-c7ccccc7)cc6)c6ccc(-c7ccccc7)cc6)cc5)ccc4c23)c2ccccc2-c2ccccc21]

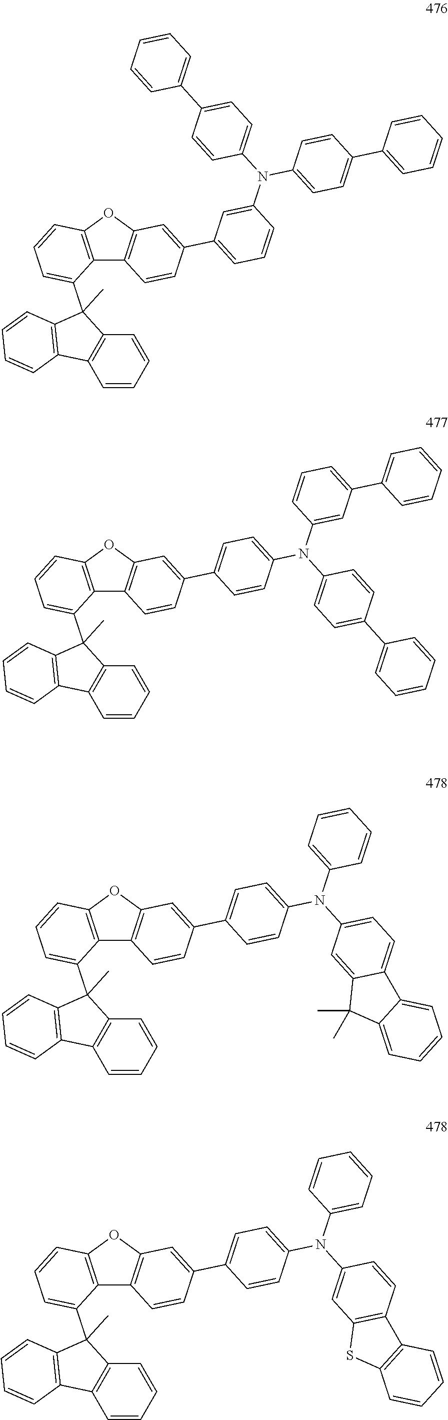 [SMILES: CC1(c2cccc3oc4cc(-c5cccc(N(c6ccc(-c7ccccc7)cc6)c6ccc(-c7ccccc7)cc6)c5)ccc4c23)c2ccccc2-c2ccccc21 ; CC1(c2cccc3oc4cc(-c5ccc(N(c6ccc(-c7ccccc7)cc6)c6cccc(-c7ccccc7)c6)cc5)ccc4c23)c2ccccc2-c2ccccc21 ; CC1(C)c2ccccc2-c2ccc(N(c3ccccc3)c3ccc(-c4ccc5c(c4)oc4cccc(C6(C)c7ccccc7-c7ccccc76)c45)cc3)cc21 ; CC1(c2cccc3oc4cc(-c5ccc(N(c6ccccc6)c6ccc7c(c6)sc6ccccc67)cc5)ccc4c23)c2ccccc2-c2ccccc21]

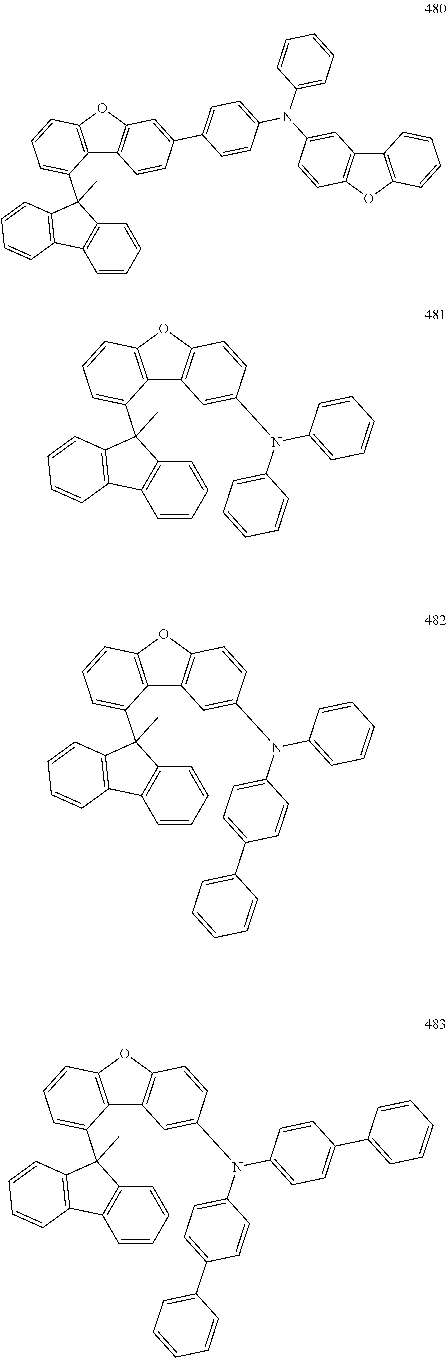 [SMILES: CC1(c2cccc3oc4cc(-c5ccc(N(c6ccccc6)c6ccc7oc8ccccc8c7c6)cc5)ccc4c23)c2ccccc2-c2ccccc21 ; CC1(c2cccc3oc4ccc(N(c5ccccc5)c5ccccc5)cc4c23)c2ccccc2-c2ccccc21 ; CC1(c2cccc3oc4ccc(N(c5ccccc5)c5ccc(-c6ccccc6)cc5)cc4c23)c2ccccc2-c2ccccc21 ; CC1(c2cccc3oc4ccc(N(c5ccc(-c6ccccc6)cc5)c5ccc(-c6ccccc6)cc5)cc4c23)c2ccccc2-c2ccccc21]

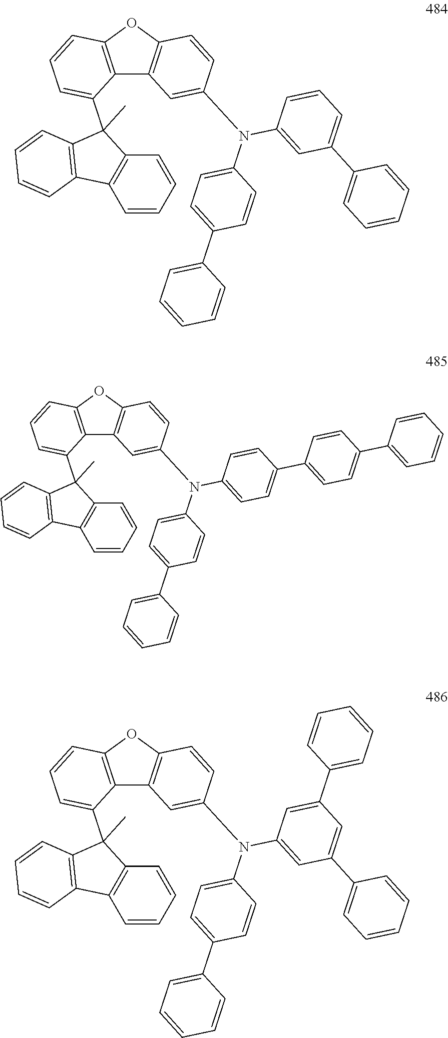 [SMILES: CC1(c2cccc3oc4ccc(N(c5ccc(-c6ccccc6)cc5)c5cccc(-c6ccccc6)c5)cc4c23)c2ccccc2-c2ccccc21 ; CC1(c2cccc3oc4ccc(N(c5ccc(-c6ccccc6)cc5)c5ccc(-c6ccc(-c7ccccc7)cc6)cc5)cc4c23)c2ccccc2-c2ccccc21 ; CC1(c2cccc3oc4ccc(N(c5ccc(-c6ccccc6)cc5)c5cc(-c6ccccc6)cc(-c6ccccc6)c5)cc4c23)c2ccccc2-c2ccccc21]

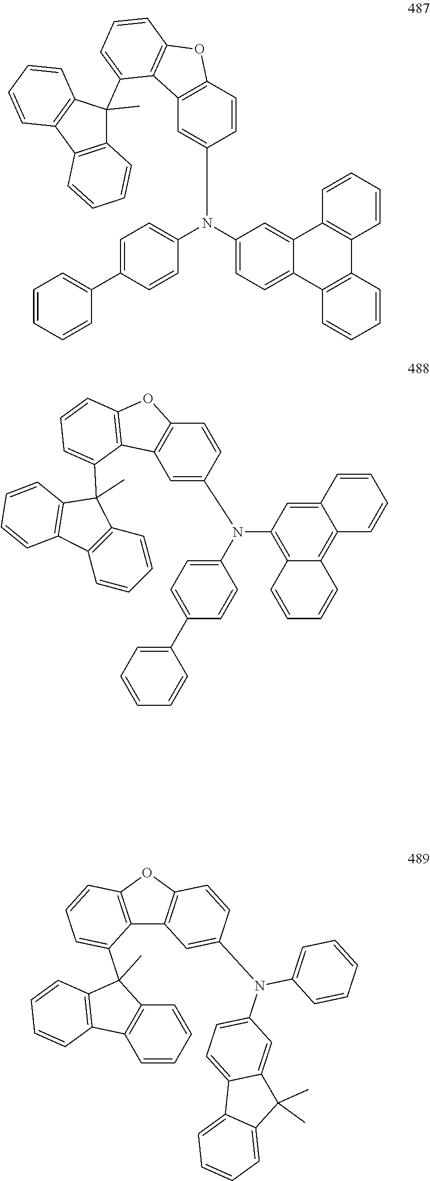 [SMILES: CC1(c2cccc3oc4ccc(N(c5ccc(-c6ccccc6)cc5)c5ccc6c7ccccc7c7ccccc7c6c5)cc4c23)c2ccccc2-c2ccccc21 ; CC1(c2cccc3oc4ccc(N(c5ccc(-c6ccccc6)cc5)c5cc6ccccc6c6ccccc56)cc4c23)c2ccccc2-c2ccccc21 ; CC1(C)c2ccccc2-c2ccc(N(c3ccccc3)c3ccc4oc5cccc(C6(C)c7ccccc7-c7ccccc76)c5c4c3)cc21]

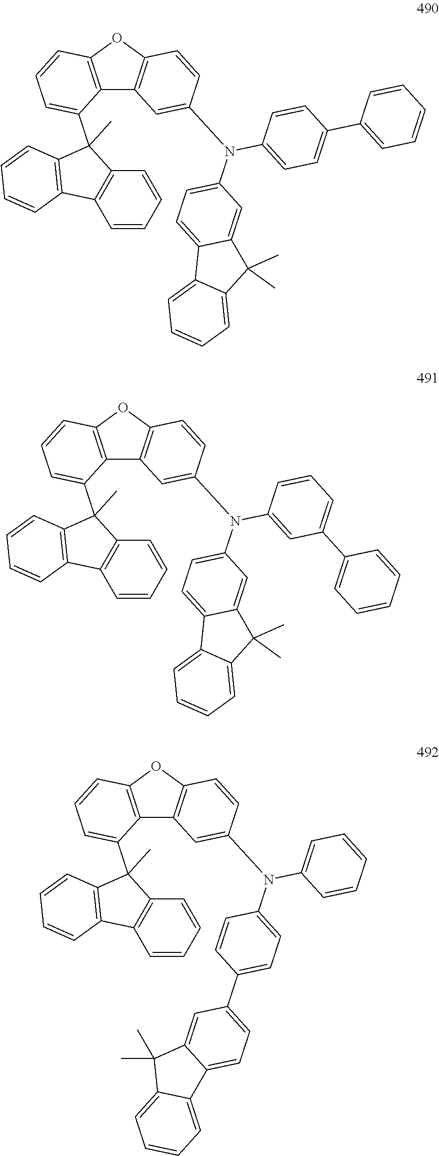 [SMILES: CC1(C)c2ccccc2-c2ccc(N(c3ccc(-c4ccccc4)cc3)c3ccc4oc5cccc(C6(C)c7ccccc7-c7ccccc76)c5c4c3)cc21 ; CC1(C)c2ccccc2-c2ccc(N(c3cccc(-c4ccccc4)c3)c3ccc4oc5cccc(C6(C)c7ccccc7-c7ccccc76)c5c4c3)cc21 ; CC1(C)c2ccccc2-c2ccc(-c3ccc(N(c4ccccc4)c4ccc5oc6cccc(C7(C)c8ccccc8-c8ccccc87)c6c5c4)cc3)cc21]

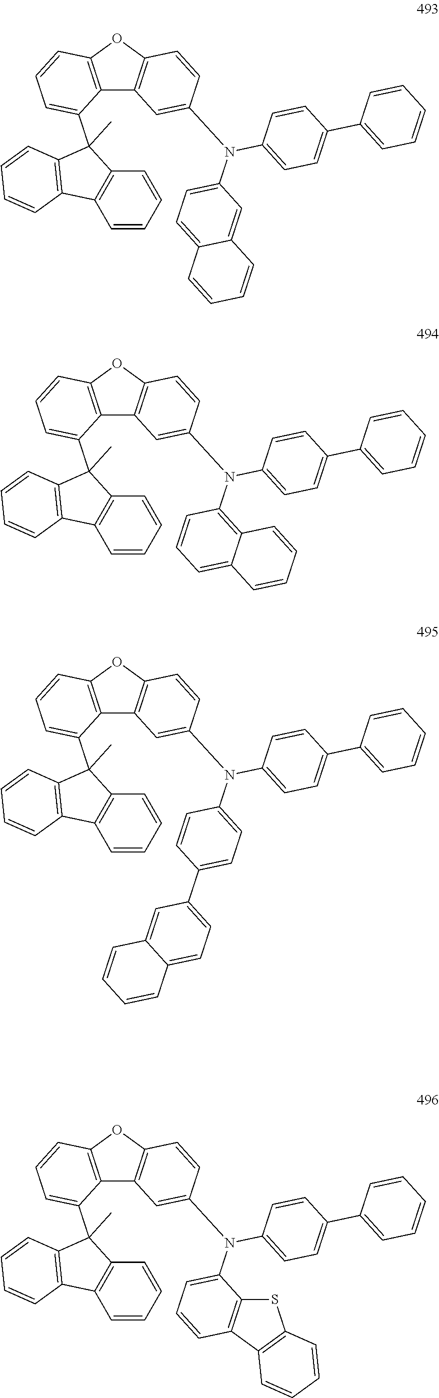 [SMILES: CC1(c2cccc3oc4ccc(N(c5ccc(-c6ccccc6)cc5)c5ccc6ccccc6c5)cc4c23)c2ccccc2-c2ccccc21 ; CC1(c2cccc3oc4ccc(N(c5ccc(-c6ccccc6)cc5)c5cccc6ccccc56)cc4c23)c2ccccc2-c2ccccc21 ; CC1(c2cccc3oc4ccc(N(c5ccc(-c6ccccc6)cc5)c5ccc(-c6ccc7ccccc7c6)cc5)cc4c23)c2ccccc2-c2ccccc21 ; CC1(c2cccc3oc4ccc(N(c5ccc(-c6ccccc6)cc5)c5cccc6c5sc5ccccc56)cc4c23)c2ccccc2-c2ccccc21]

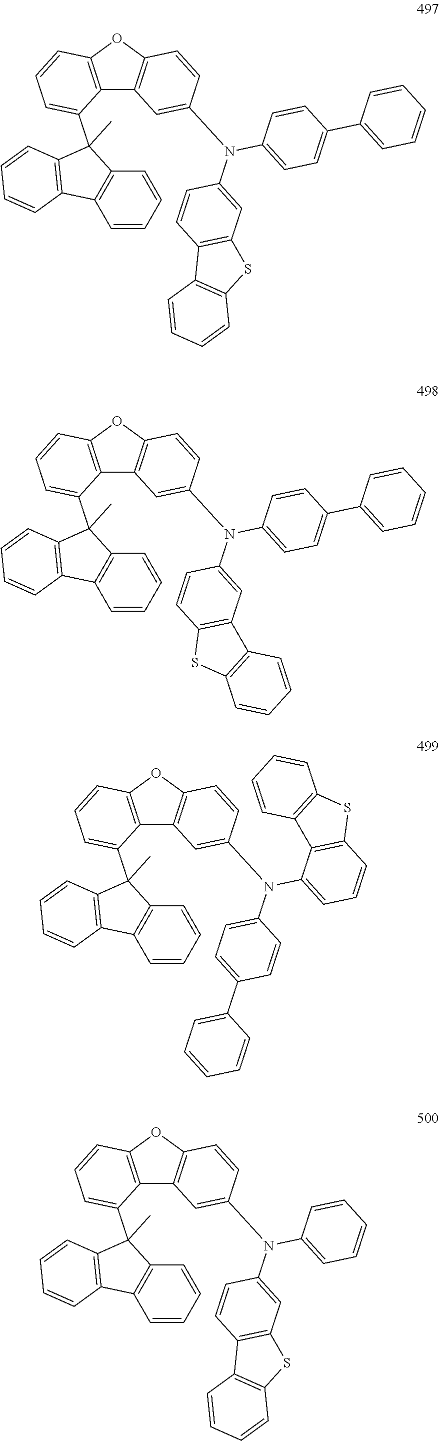

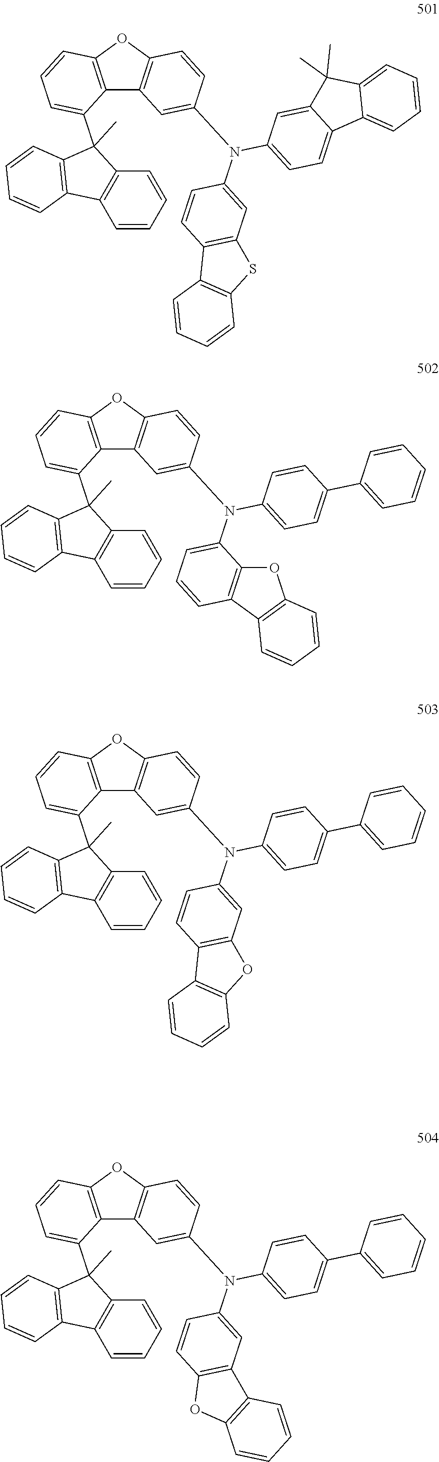 [SMILES: CC1(C)c2ccccc2-c2ccc(N(c3ccc4c(c3)sc3ccccc34)c3ccc4oc5cccc(C6(C)c7ccccc7-c7ccccc76)c5c4c3)cc21 ; CC1(c2cccc3oc4ccc(N(c5ccc(-c6ccccc6)cc5)c5cccc6c5oc5ccccc56)cc4c23)c2ccccc2-c2ccccc21 ; CC1(c2cccc3oc4ccc(N(c5ccc(-c6ccccc6)cc5)c5ccc6c(c5)oc5ccccc56)cc4c23)c2ccccc2-c2ccccc21 ; CC1(c2cccc3oc4ccc(N(c5ccc(-c6ccccc6)cc5)c5ccc6oc7ccccc7c6c5)cc4c23)c2ccccc2-c2ccccc21]

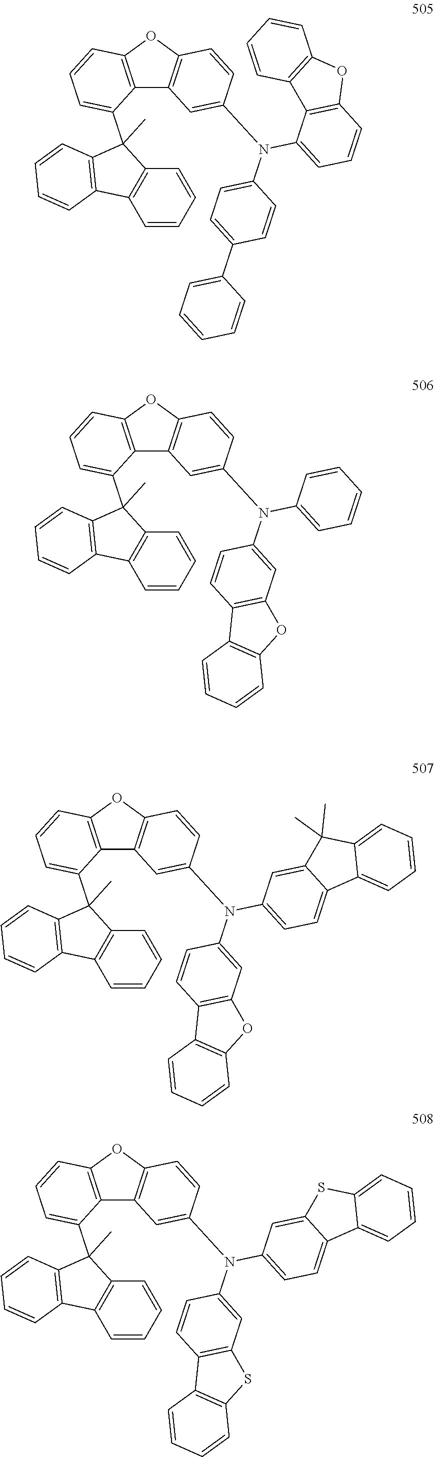 [SMILES: CC1(c2cccc3oc4ccc(N(c5ccc(-c6ccccc6)cc5)c5cccc6oc7ccccc7c56)cc4c23)c2ccccc2-c2ccccc21 ; CC1(c2cccc3oc4ccc(N(c5ccccc5)c5ccc6c(c5)oc5ccccc56)cc4c23)c2ccccc2-c2ccccc21 ; CC1(C)c2ccccc2-c2ccc(N(c3ccc4c(c3)oc3ccccc34)c3ccc4oc5cccc(C6(C)c7ccccc7-c7ccccc76)c5c4c3)cc21 ; CC1(c2cccc3oc4ccc(N(c5ccc6c(c5)sc5ccccc56)c5ccc6c(c5)sc5ccccc56)cc4c23)c2ccccc2-c2ccccc21]

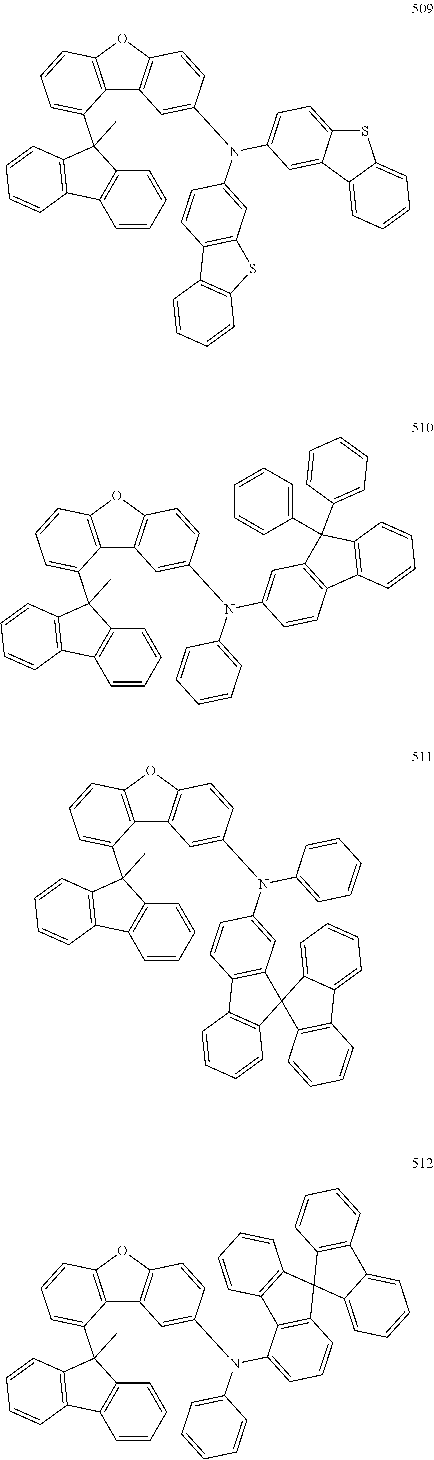 [SMILES: CC1(c2cccc3oc4ccc(N(c5ccc6c(c5)sc5ccccc56)c5ccc6sc7ccccc7c6c5)cc4c23)c2ccccc2-c2ccccc21 ; CC1(c2cccc3oc4ccc(N(c5ccccc5)c5ccc6c(c5)C(c5ccccc5)(c5ccccc5)c5ccccc5-6)cc4c23)c2ccccc2-c2ccccc21 ; CC1(c2cccc3oc4ccc(N(c5ccccc5)c5ccc6c(c5)C5(c7ccccc7-c7ccccc75)c5ccccc5-6)cc4c23)c2ccccc2-c2ccccc21 ; CC1(c2cccc3oc4ccc(N(c5ccccc5)c5cccc6c5-c5ccccc5C65c6ccccc6-c6ccccc65)cc4c23)c2ccccc2-c2ccccc21]

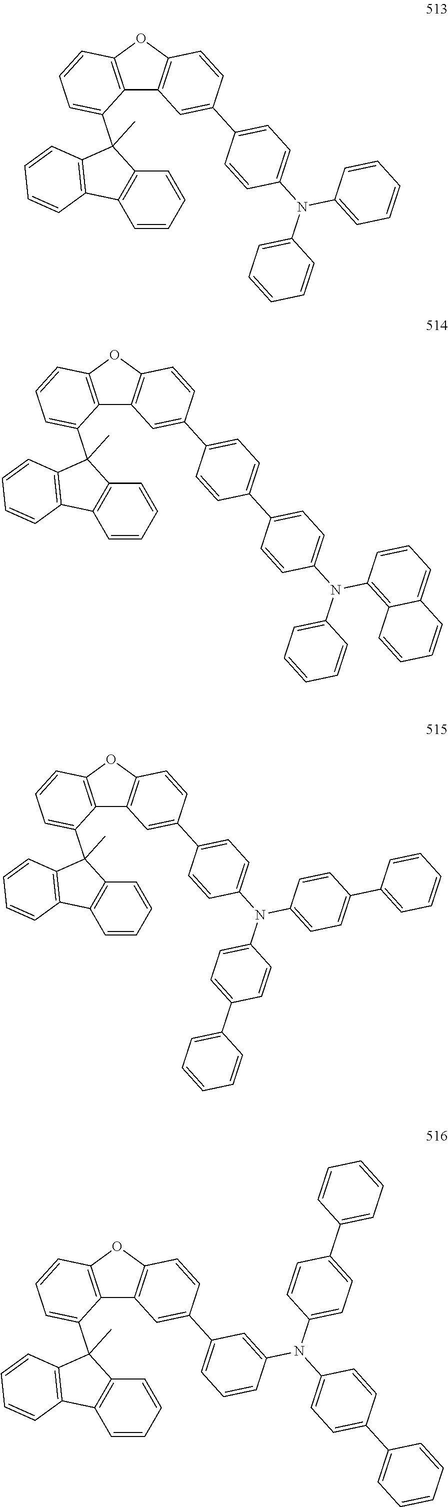 [SMILES: CC1(c2cccc3oc4ccc(-c5ccc(N(c6ccccc6)c6ccccc6)cc5)cc4c23)c2ccccc2-c2ccccc21 ; CC1(c2cccc3oc4ccc(-c5ccc(-c6ccc(N(c7ccccc7)c7cccc8ccccc78)cc6)cc5)cc4c23)c2ccccc2-c2ccccc21 ; CC1(c2cccc3oc4ccc(-c5ccc(N(c6ccc(-c7ccccc7)cc6)c6ccc(-c7ccccc7)cc6)cc5)cc4c23)c2ccccc2-c2ccccc21 ; CC1(c2cccc3oc4ccc(-c5cccc(N(c6ccc(-c7ccccc7)cc6)c6ccc(-c7ccccc7)cc6)c5)cc4c23)c2ccccc2-c2ccccc21]

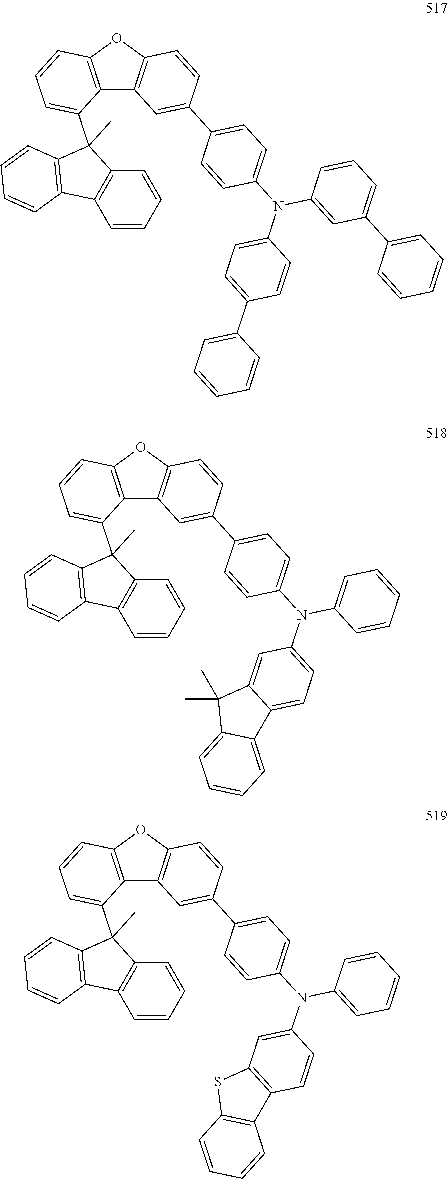 [SMILES: CC1(c2cccc3oc4ccc(-c5ccc(N(c6ccc(-c7ccccc7)cc6)c6cccc(-c7ccccc7)c6)cc5)cc4c23)c2ccccc2-c2ccccc21 ; CC1(C)c2ccccc2-c2ccc(N(c3ccccc3)c3ccc(-c4ccc5oc6cccc(C7(C)c8ccccc8-c8ccccc87)c6c5c4)cc3)cc21 ; CC1(c2cccc3oc4ccc(-c5ccc(N(c6ccccc6)c6ccc7c(c6)sc6ccccc67)cc5)cc4c23)c2ccccc2-c2ccccc21]

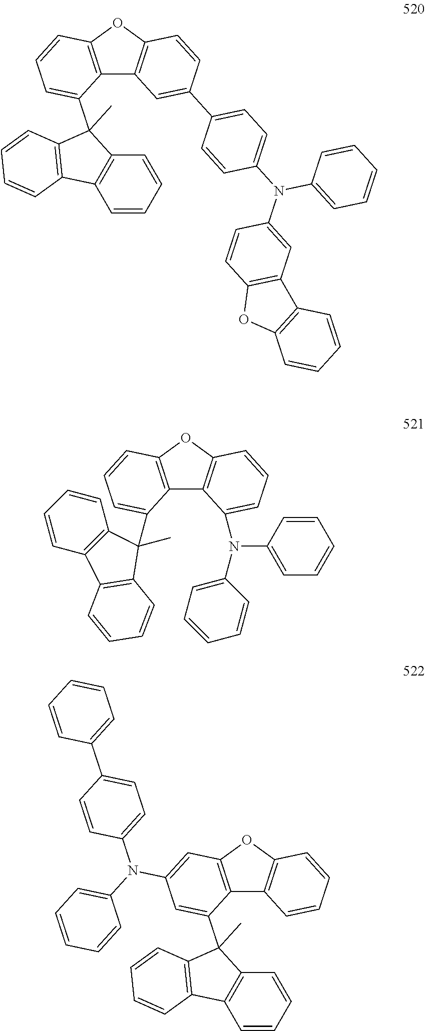

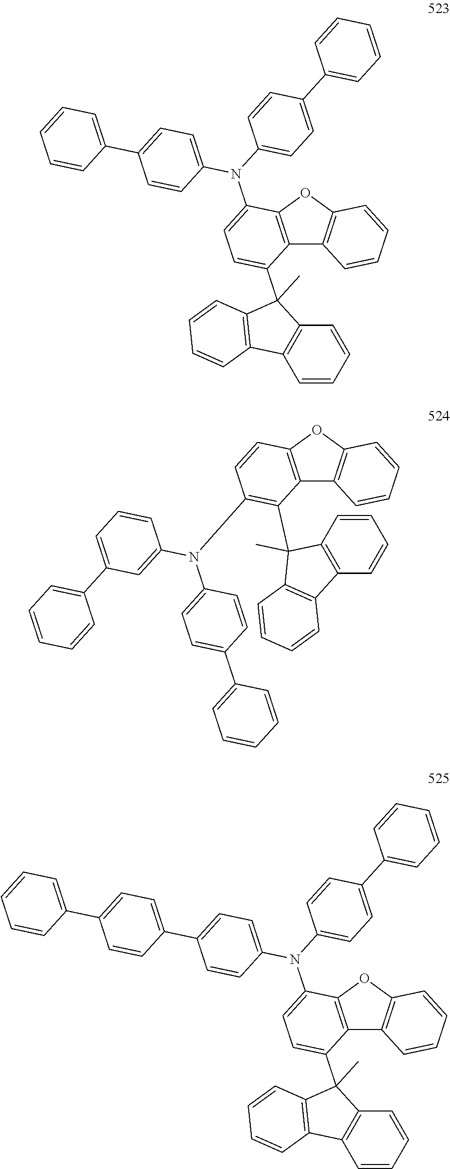 [SMILES: CC1(c2ccc(N(c3ccc(-c4ccccc4)cc3)c3ccc(-c4ccccc4)cc3)c3oc4ccccc4c23)c2ccccc2-c2ccccc21 ; CC1(c2c(N(c3ccc(-c4ccccc4)cc3)c3cccc(-c4ccccc4)c3)ccc3oc4ccccc4c23)c2ccccc2-c2ccccc21 ; CC1(c2ccc(N(c3ccc(-c4ccccc4)cc3)c3ccc(-c4ccc(-c5ccccc5)cc4)cc3)c3oc4ccccc4c23)c2ccccc2-c2ccccc21]

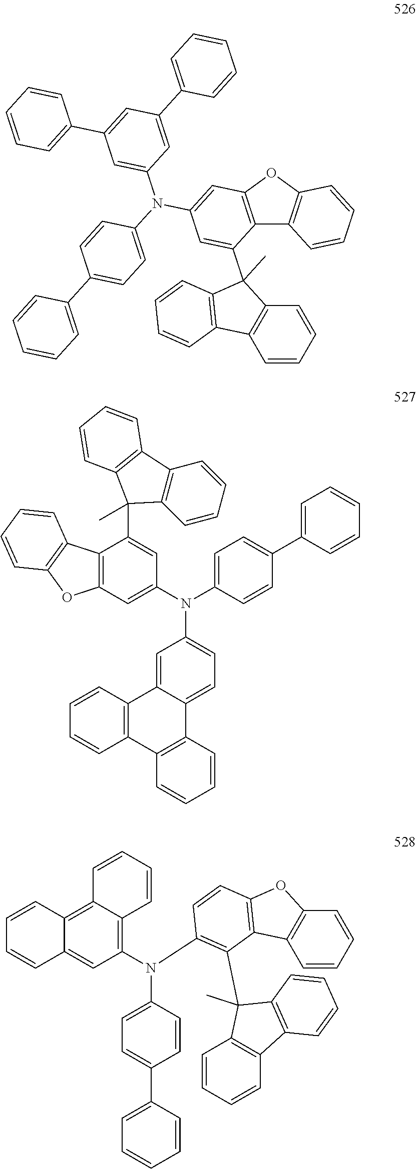 [SMILES: CC1(c2cc(N(c3ccc(-c4ccccc4)cc3)c3cc(-c4ccccc4)cc(-c4ccccc4)c3)cc3oc4ccccc4c23)c2ccccc2-c2ccccc21 ; CC1(c2cc(N(c3ccc(-c4ccccc4)cc3)c3ccc4c5ccccc5c5ccccc5c4c3)cc3oc4ccccc4c23)c2ccccc2-c2ccccc21 ; CC1(c2c(N(c3ccc(-c4ccccc4)cc3)c3cc4ccccc4c4ccccc34)ccc3oc4ccccc4c23)c2ccccc2-c2ccccc21]

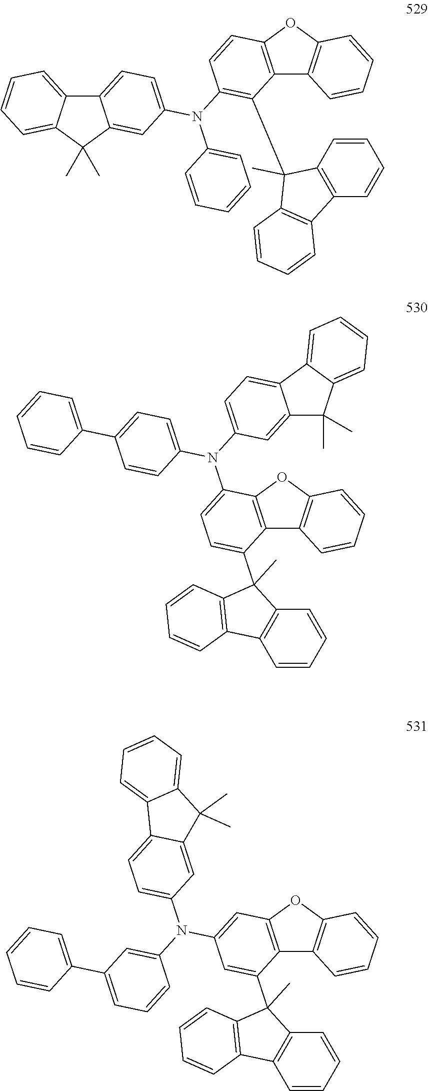 [SMILES: CC1(C)c2ccccc2-c2ccc(N(c3ccccc3)c3ccc4oc5ccccc5c4c3C3(C)c4ccccc4-c4ccccc43)cc21 ; CC1(C)c2ccccc2-c2ccc(N(c3ccc(-c4ccccc4)cc3)c3ccc(C4(C)c5ccccc5-c5ccccc54)c4c3oc3ccccc34)cc21 ; CC1(C)c2ccccc2-c2ccc(N(c3cccc(-c4ccccc4)c3)c3cc(C4(C)c5ccccc5-c5ccccc54)c4c(c3)oc3ccccc34)cc21]

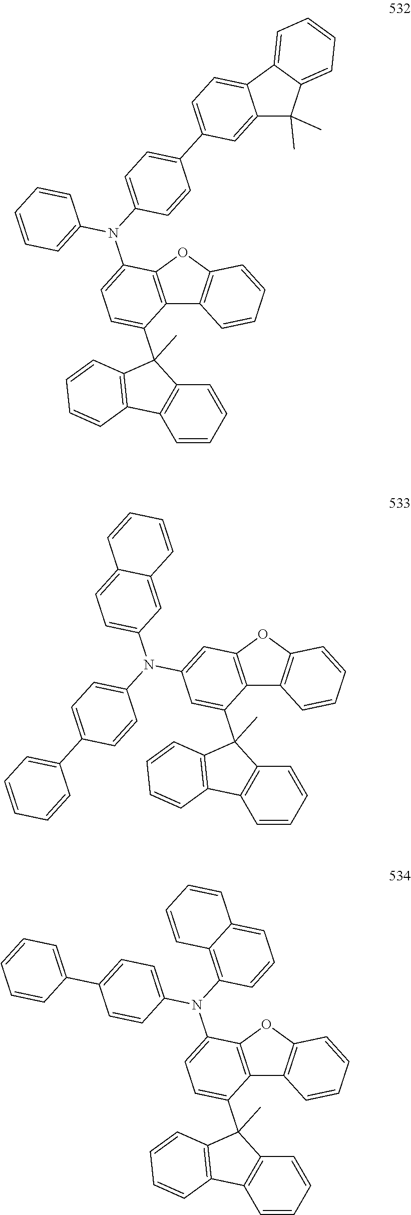 [SMILES: CC1(C)c2ccccc2-c2ccc(-c3ccc(N(c4ccccc4)c4ccc(C5(C)c6ccccc6-c6ccccc65)c5c4oc4ccccc45)cc3)cc21 ; CC1(c2cc(N(c3ccc(-c4ccccc4)cc3)c3ccc4ccccc4c3)cc3oc4ccccc4c23)c2ccccc2-c2ccccc21 ; CC1(c2ccc(N(c3ccc(-c4ccccc4)cc3)c3cccc4ccccc34)c3oc4ccccc4c23)c2ccccc2-c2ccccc21]

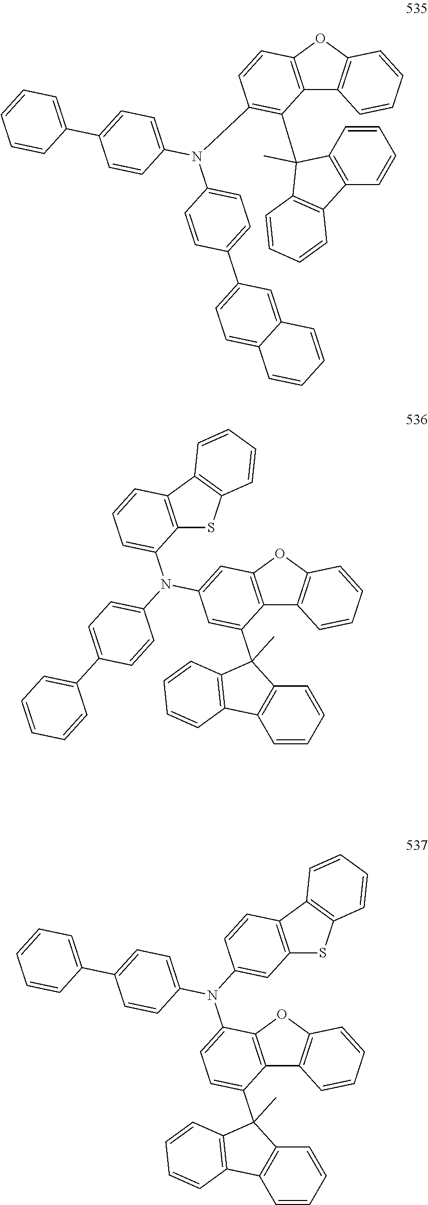 [SMILES: CC1(c2c(N(c3ccc(-c4ccccc4)cc3)c3ccc(-c4ccc5ccccc5c4)cc3)ccc3oc4ccccc4c23)c2ccccc2-c2ccccc21 ; CC1(c2cc(N(c3ccc(-c4ccccc4)cc3)c3cccc4c3sc3ccccc34)cc3oc4ccccc4c23)c2ccccc2-c2ccccc21 ; CC1(c2ccc(N(c3ccc(-c4ccccc4)cc3)c3ccc4c(c3)sc3ccccc34)c3oc4ccccc4c23)c2ccccc2-c2ccccc21]

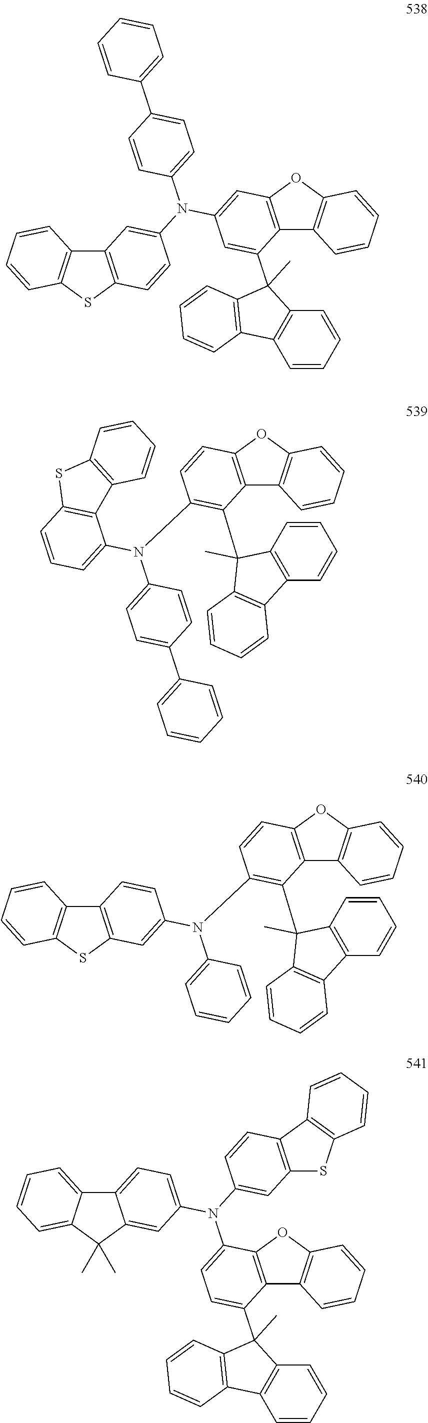 [SMILES: CC1(c2cc(N(c3ccc(-c4ccccc4)cc3)c3ccc4sc5ccccc5c4c3)cc3oc4ccccc4c23)c2ccccc2-c2ccccc21 ; CC1(c2c(N(c3ccc(-c4ccccc4)cc3)c3cccc4sc5ccccc5c34)ccc3oc4ccccc4c23)c2ccccc2-c2ccccc21 ; CC1(c2c(N(c3ccccc3)c3ccc4c(c3)sc3ccccc34)ccc3oc4ccccc4c23)c2ccccc2-c2ccccc21 ; CC1(C)c2ccccc2-c2ccc(N(c3ccc4c(c3)sc3ccccc34)c3ccc(C4(C)c5ccccc5-c5ccccc54)c4c3oc3ccccc34)cc21]

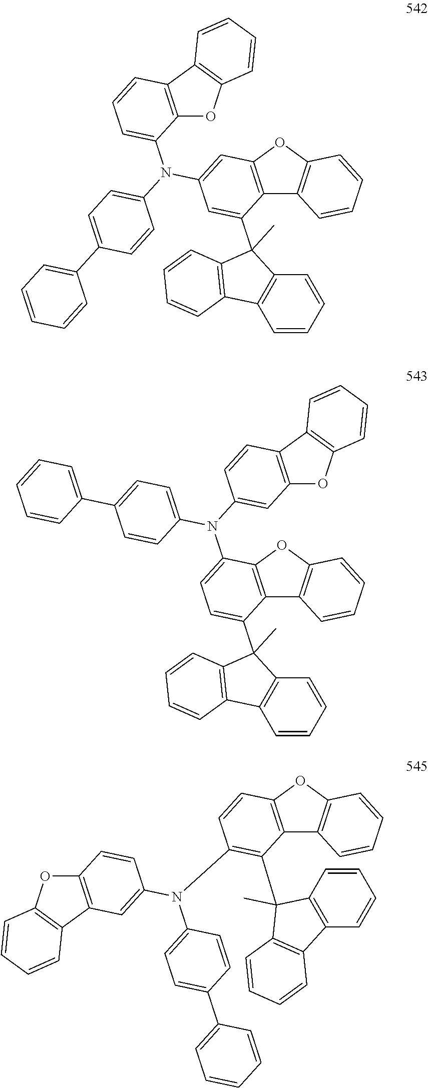

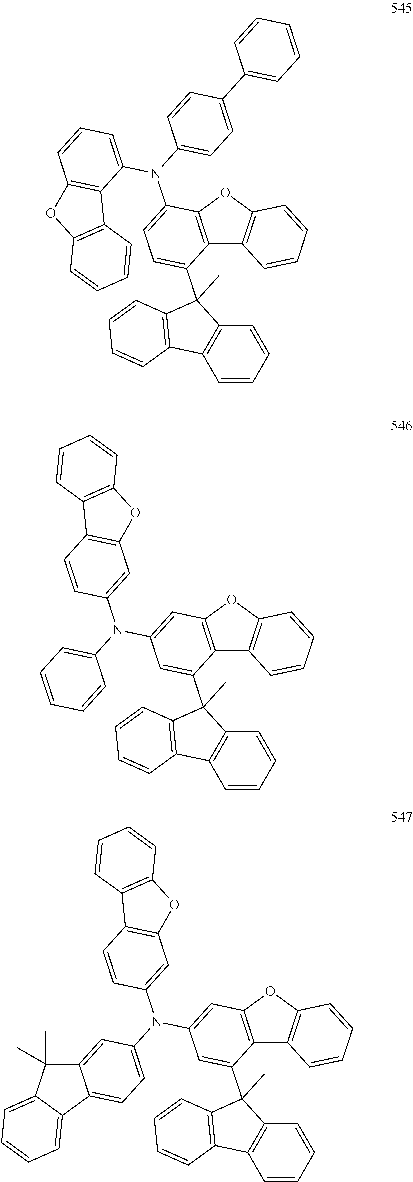 [SMILES: CC1(c2ccc(N(c3ccc(-c4ccccc4)cc3)c3cccc4oc5ccccc5c34)c3oc4ccccc4c23)c2ccccc2-c2ccccc21 ; CC1(c2cc(N(c3ccccc3)c3ccc4c(c3)oc3ccccc34)cc3oc4ccccc4c23)c2ccccc2-c2ccccc21 ; CC1(C)c2ccccc2-c2ccc(N(c3ccc4c(c3)oc3ccccc34)c3cc(C4(C)c5ccccc5-c5ccccc54)c4c(c3)oc3ccccc34)cc21]

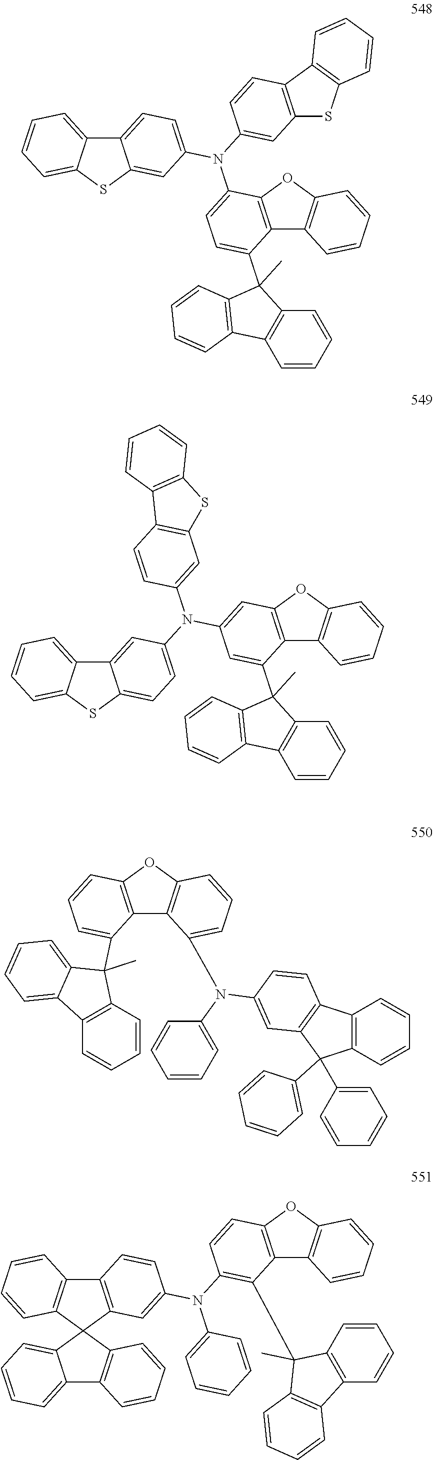 [SMILES: CC1(c2ccc(N(c3ccc4c(c3)sc3ccccc34)c3ccc4c(c3)sc3ccccc34)c3oc4ccccc4c23)c2ccccc2-c2ccccc21 ; CC1(c2cc(N(c3ccc4c(c3)sc3ccccc34)c3ccc4sc5ccccc5c4c3)cc3oc4ccccc4c23)c2ccccc2-c2ccccc21 ; CC1(c2cccc3oc4cccc(N(c5ccccc5)c5ccc6c(c5)C(c5ccccc5)(c5ccccc5)c5ccccc5-6)c4c23)c2ccccc2-c2ccccc21 ; CC1(c2c(N(c3ccccc3)c3ccc4c(c3)C3(c5ccccc5-c5ccccc53)c3ccccc3-4)ccc3oc4ccccc4c23)c2ccccc2-c2ccccc21]

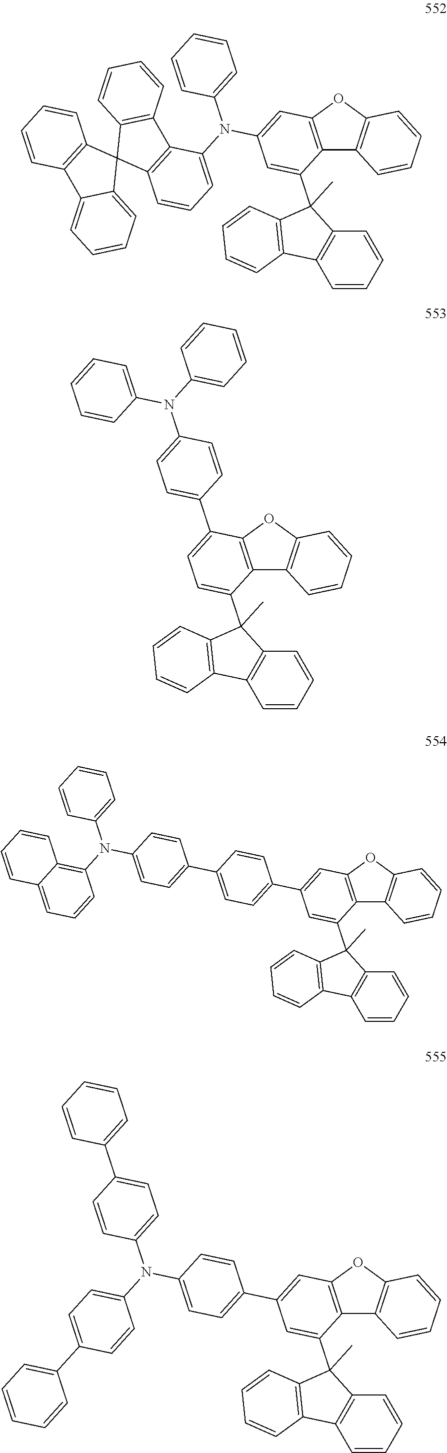 [SMILES: CC1(c2cc(N(c3ccccc3)c3cccc4c3-c3ccccc3C43c4ccccc4-c4ccccc43)cc3oc4ccccc4c23)c2ccccc2-c2ccccc21 ; CC1(c2ccc(-c3ccc(N(c4ccccc4)c4ccccc4)cc3)c3oc4ccccc4c23)c2ccccc2-c2ccccc21 ; CC1(c2cc(-c3ccc(-c4ccc(N(c5ccccc5)c5cccc6ccccc56)cc4)cc3)cc3oc4ccccc4c23)c2ccccc2-c2ccccc21 ; CC1(c2cc(-c3ccc(N(c4ccc(-c5ccccc5)cc4)c4ccc(-c5ccccc5)cc4)cc3)cc3oc4ccccc4c23)c2ccccc2-c2ccccc21]

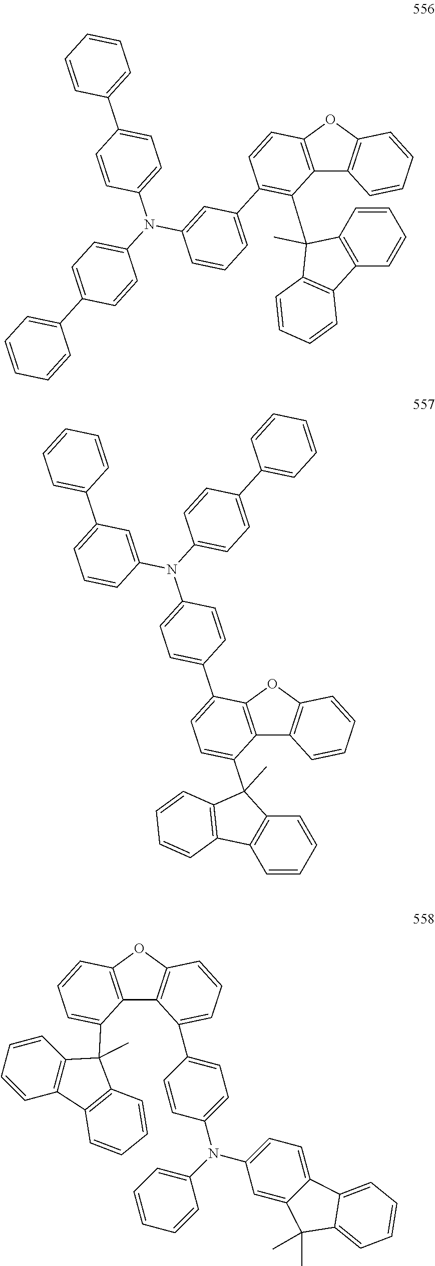 [SMILES: CC1(c2c(-c3cccc(N(c4ccc(-c5ccccc5)cc4)c4ccc(-c5ccccc5)cc4)c3)ccc3oc4ccccc4c23)c2ccccc2-c2ccccc21 ; CC1(c2ccc(-c3ccc(N(c4ccc(-c5ccccc5)cc4)c4cccc(-c5ccccc5)c4)cc3)c3oc4ccccc4c23)c2ccccc2-c2ccccc21 ; CC1(C)c2ccccc2-c2ccc(N(c3ccccc3)c3ccc(-c4cccc5oc6cccc(C7(C)c8ccccc8-c8ccccc87)c6c45)cc3)cc21]

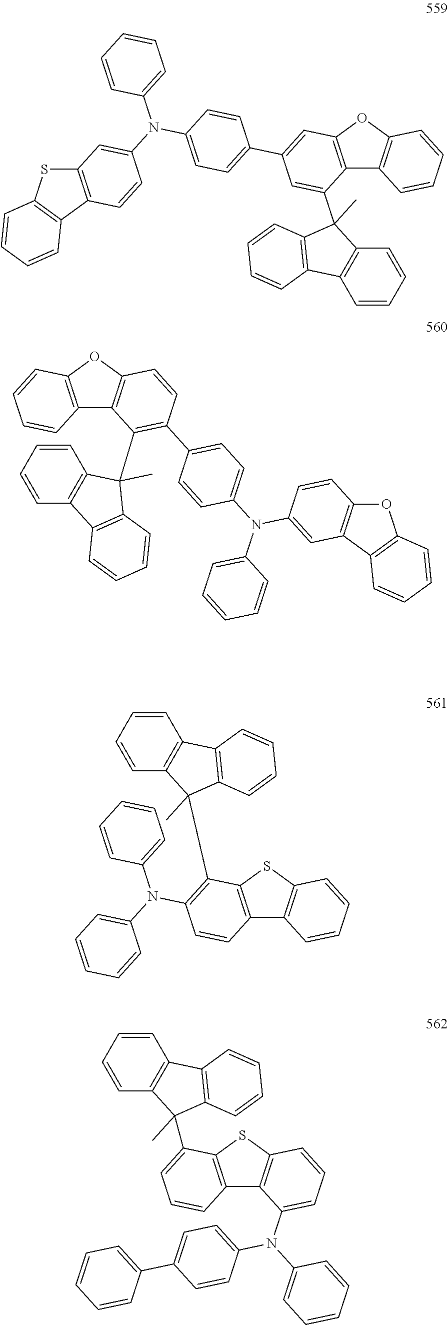 [SMILES: CC1(c2cc(-c3ccc(N(c4ccccc4)c4ccc5c(c4)sc4ccccc45)cc3)cc3oc4ccccc4c23)c2ccccc2-c2ccccc21 ; CC1(c2c(-c3ccc(N(c4ccccc4)c4ccc5oc6ccccc6c5c4)cc3)ccc3oc4ccccc4c23)c2ccccc2-c2ccccc21 ; CC1(c2c(N(c3ccccc3)c3ccccc3)ccc3c2sc2ccccc23)c2ccccc2-c2ccccc21 ; CC1(c2cccc3c2sc2cccc(N(c4ccccc4)c4ccc(-c5ccccc5)cc4)c23)c2ccccc2-c2ccccc21]

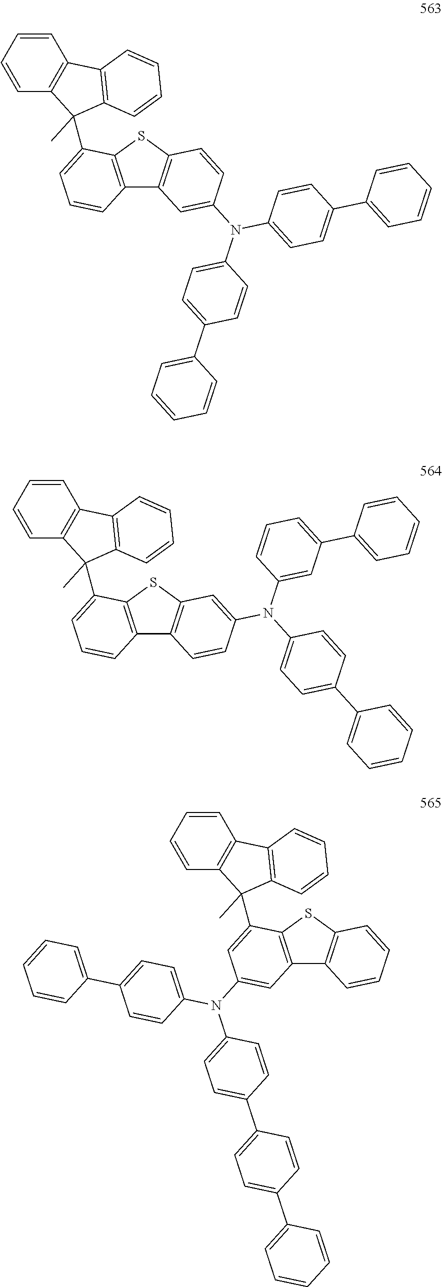 [SMILES: CC1(c2cccc3c2sc2ccc(N(c4ccc(-c5ccccc5)cc4)c4ccc(-c5ccccc5)cc4)cc23)c2ccccc2-c2ccccc21 ; CC1(c2cccc3c2sc2cc(N(c4ccc(-c5ccccc5)cc4)c4cccc(-c5ccccc5)c4)ccc23)c2ccccc2-c2ccccc21 ; CC1(c2cc(N(c3ccc(-c4ccccc4)cc3)c3ccc(-c4ccc(-c5ccccc5)cc4)cc3)cc3c2sc2ccccc23)c2ccccc2-c2ccccc21]

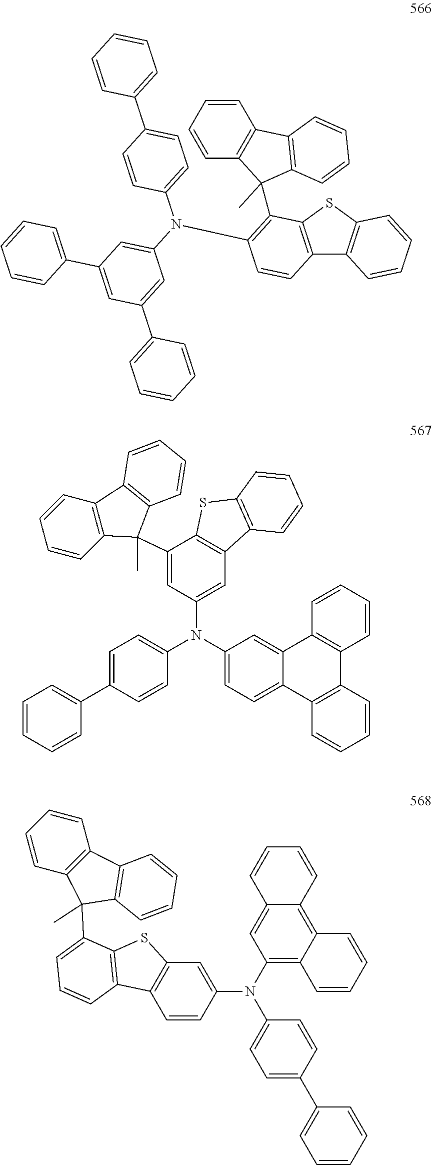

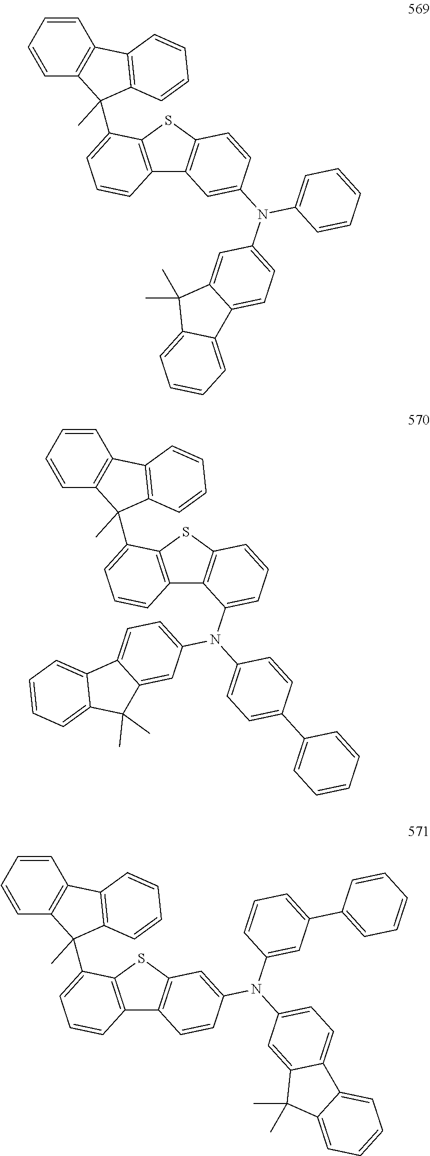 [SMILES: CC1(C)c2ccccc2-c2ccc(N(c3ccccc3)c3ccc4sc5c(C6(C)c7ccccc7-c7ccccc76)cccc5c4c3)cc21 ; CC1(C)c2ccccc2-c2ccc(N(c3ccc(-c4ccccc4)cc3)c3cccc4sc5c(C6(C)c7ccccc7-c7ccccc76)cccc5c34)cc21 ; CC1(C)c2ccccc2-c2ccc(N(c3cccc(-c4ccccc4)c3)c3ccc4c(c3)sc3c(C5(C)c6ccccc6-c6ccccc65)cccc34)cc21]

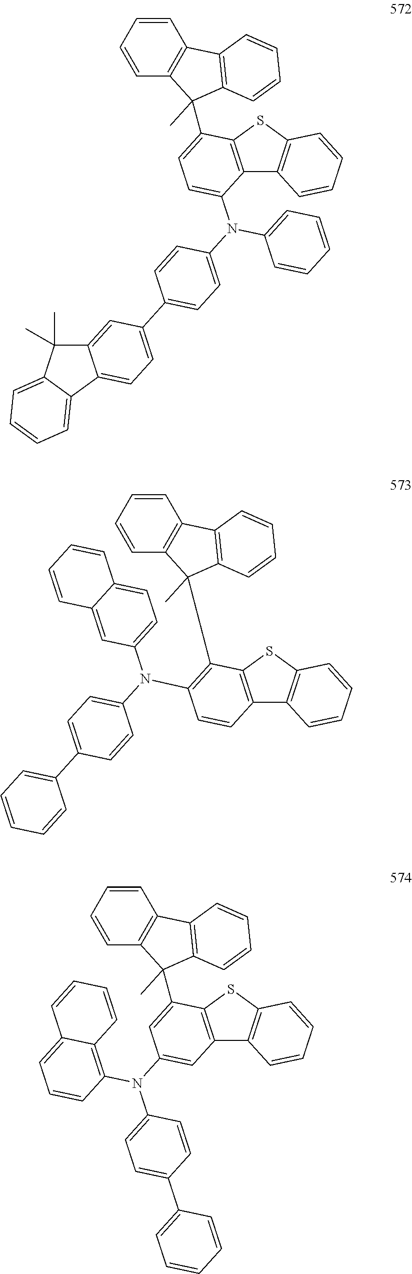 [SMILES: CC1(C)c2ccccc2-c2ccc(-c3ccc(N(c4ccccc4)c4ccc(C5(C)c6ccccc6-c6ccccc65)c5sc6ccccc6c45)cc3)cc21 ; CC1(c2c(N(c3ccc(-c4ccccc4)cc3)c3ccc4ccccc4c3)ccc3c2sc2ccccc23)c2ccccc2-c2ccccc21 ; CC1(c2cc(N(c3ccc(-c4ccccc4)cc3)c3cccc4ccccc34)cc3c2sc2ccccc23)c2ccccc2-c2ccccc21]

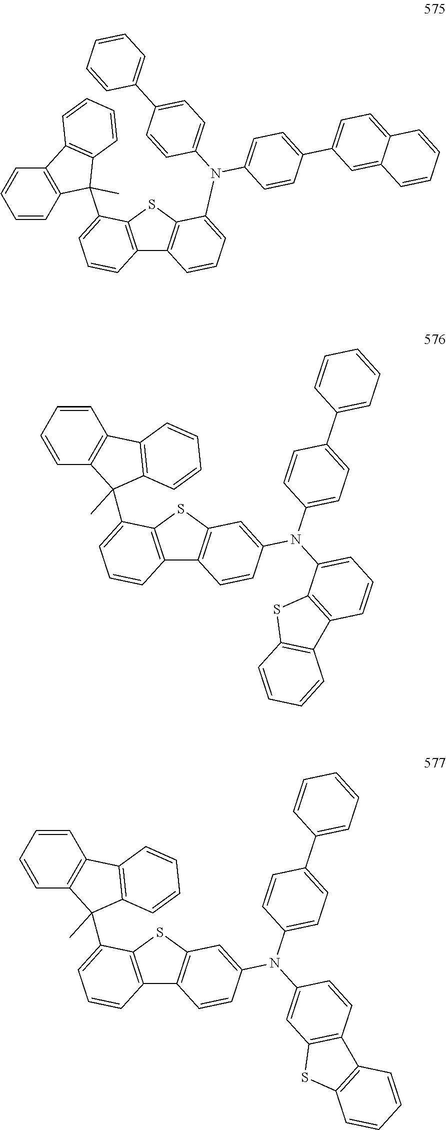 [SMILES: CC1(c2cccc3c2sc2c(N(c4ccc(-c5ccccc5)cc4)c4ccc(-c5ccc6ccccc6c5)cc4)cccc23)c2ccccc2-c2ccccc21 ; CC1(c2cccc3c2sc2cc(N(c4ccc(-c5ccccc5)cc4)c4cccc5c4sc4ccccc45)ccc23)c2ccccc2-c2ccccc21 ; CC1(c2cccc3c2sc2cc(N(c4ccc(-c5ccccc5)cc4)c4ccc5c(c4)sc4ccccc45)ccc23)c2ccccc2-c2ccccc21]

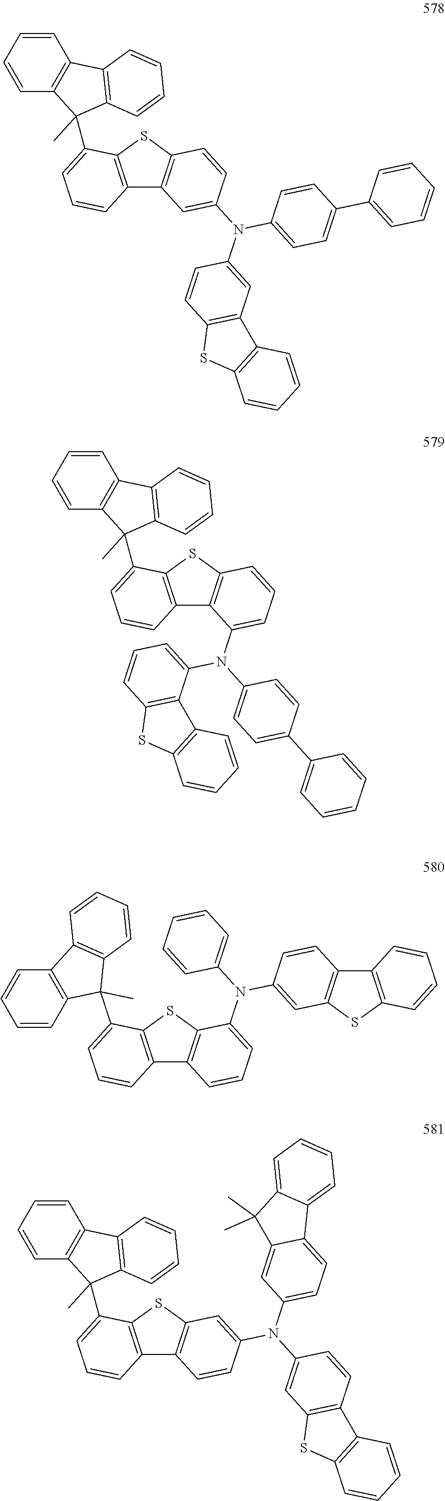 [SMILES: CC1(c2cccc3c2sc2ccc(N(c4ccc(-c5ccccc5)cc4)c4ccc5sc6ccccc6c5c4)cc23)c2ccccc2-c2ccccc21 ; CC1(c2cccc3c2sc2cccc(N(c4ccc(-c5ccccc5)cc4)c4cccc5sc6ccccc6c45)c23)c2ccccc2-c2ccccc21 ; CC1(c2cccc3c2sc2c(N(c4ccccc4)c4ccc5c(c4)sc4ccccc45)cccc23)c2ccccc2-c2ccccc21 ; CC1(C)c2ccccc2-c2ccc(N(c3ccc4c(c3)sc3ccccc34)c3ccc4c(c3)sc3c(C5(C)c6ccccc6-c6ccccc65)cccc34)cc21]

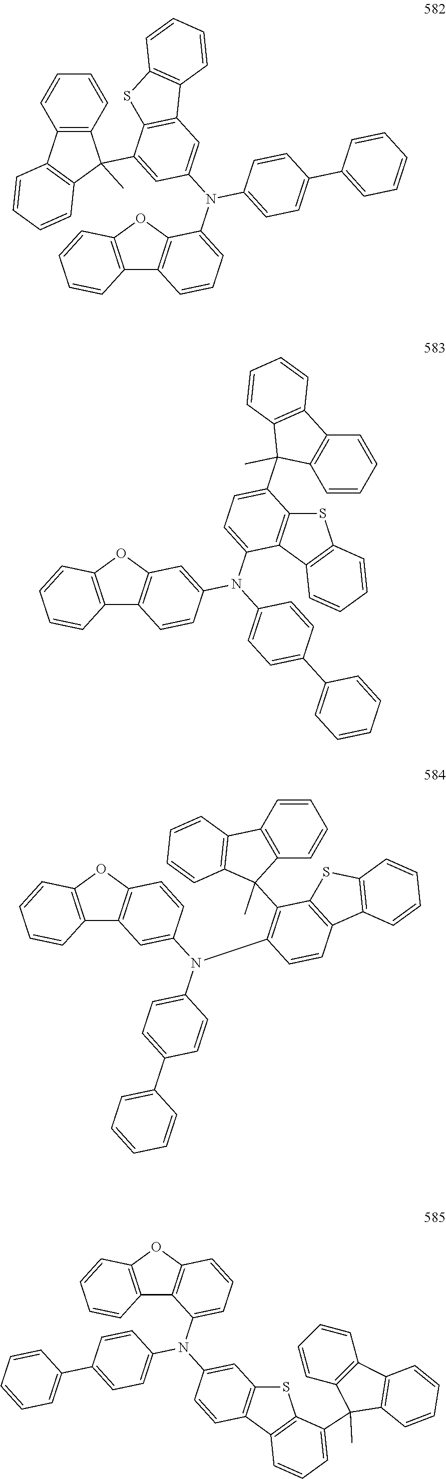 [SMILES: CC1(c2cc(N(c3ccc(-c4ccccc4)cc3)c3cccc4c3oc3ccccc34)cc3c2sc2ccccc23)c2ccccc2-c2ccccc21 ; CC1(c2ccc(N(c3ccc(-c4ccccc4)cc3)c3ccc4c(c3)oc3ccccc34)c3c2sc2ccccc23)c2ccccc2-c2ccccc21 ; CC1(c2c(N(c3ccc(-c4ccccc4)cc3)c3ccc4oc5ccccc5c4c3)ccc3c2sc2ccccc23)c2ccccc2-c2ccccc21 ; CC1(c2cccc3c2sc2cc(N(c4ccc(-c5ccccc5)cc4)c4cccc5oc6ccccc6c45)ccc23)c2ccccc2-c2ccccc21]

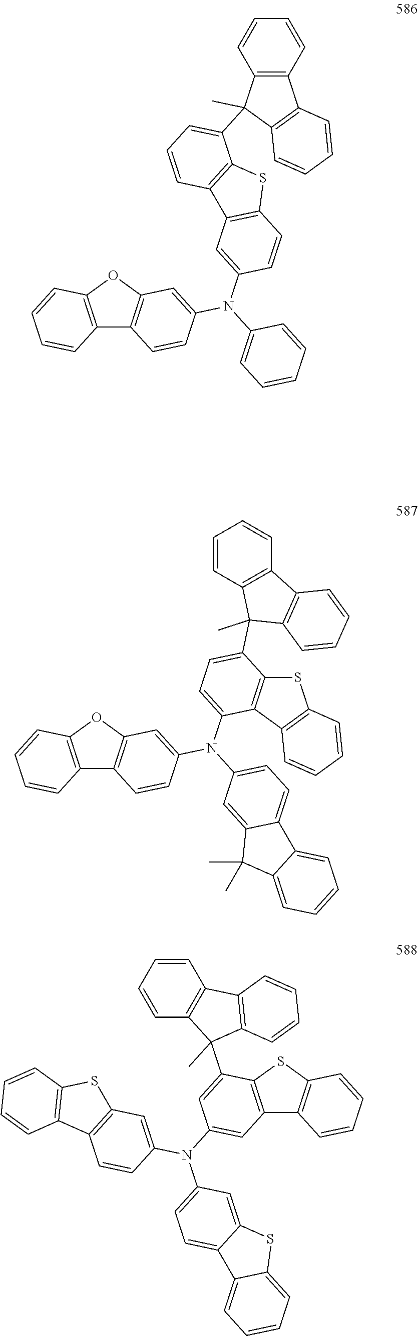 [SMILES: CC1(c2cccc3c2sc2ccc(N(c4ccccc4)c4ccc5c(c4)oc4ccccc45)cc23)c2ccccc2-c2ccccc21 ; CC1(C)c2ccccc2-c2ccc(N(c3ccc4c(c3)oc3ccccc34)c3ccc(C4(C)c5ccccc5-c5ccccc54)c4sc5ccccc5c34)cc21 ; CC1(c2cc(N(c3ccc4c(c3)sc3ccccc34)c3ccc4c(c3)sc3ccccc34)cc3c2sc2ccccc23)c2ccccc2-c2ccccc21]

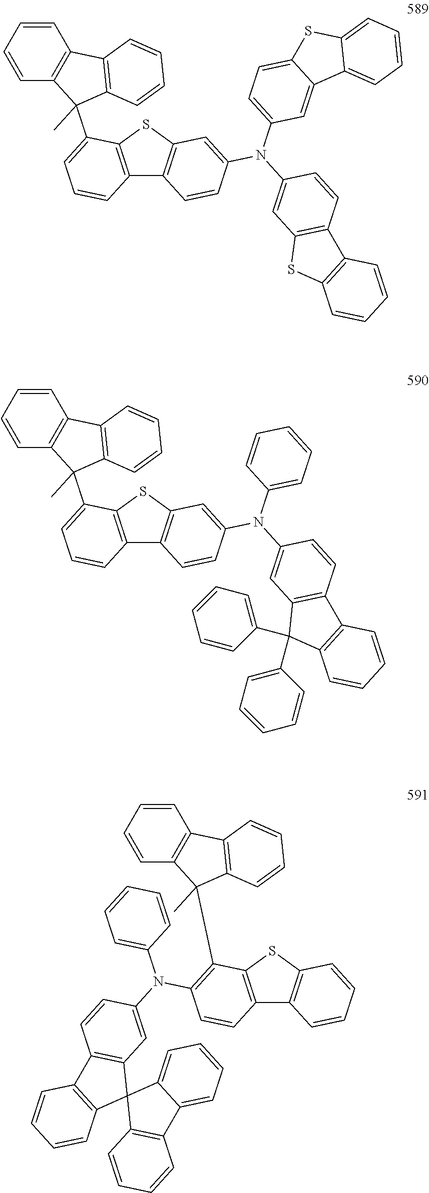 [SMILES: CC1(c2cccc3c2sc2cc(N(c4ccc5c(c4)sc4ccccc45)c4ccc5sc6ccccc6c5c4)ccc23)c2ccccc2-c2ccccc21 ; CC1(c2cccc3c2sc2cc(N(c4ccccc4)c4ccc5c(c4)C(c4ccccc4)(c4ccccc4)c4ccccc4-5)ccc23)c2ccccc2-c2ccccc21 ; CC1(c2c(N(c3ccccc3)c3ccc4c(c3)C3(c5ccccc5-c5ccccc53)c3ccccc3-4)ccc3c2sc2ccccc23)c2ccccc2-c2ccccc21]

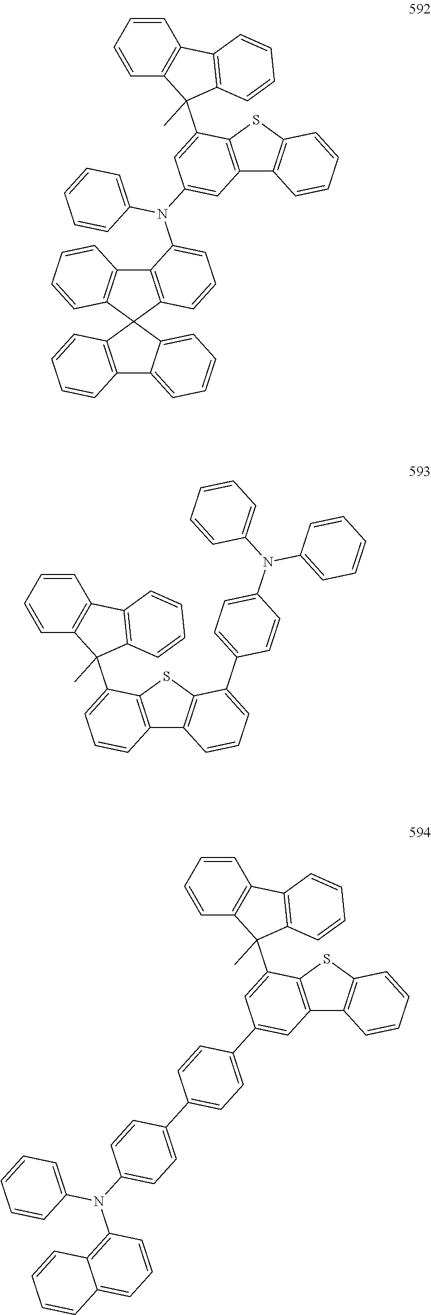 [SMILES: CC1(c2cc(N(c3ccccc3)c3cccc4c3-c3ccccc3C43c4ccccc4-c4ccccc43)cc3c2sc2ccccc23)c2ccccc2-c2ccccc21 ; CC1(c2cccc3c2sc2c(-c4ccc(N(c5ccccc5)c5ccccc5)cc4)cccc23)c2ccccc2-c2ccccc21 ; CC1(c2cc(-c3ccc(-c4ccc(N(c5ccccc5)c5cccc6ccccc56)cc4)cc3)cc3c2sc2ccccc23)c2ccccc2-c2ccccc21]

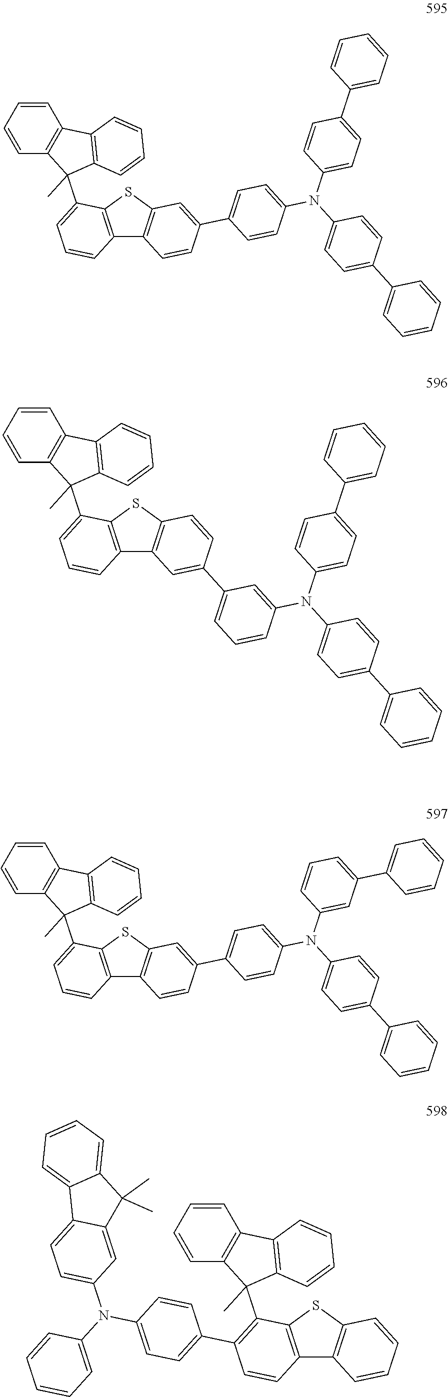 [SMILES: CC1(c2cccc3c2sc2cc(-c4ccc(N(c5ccc(-c6ccccc6)cc5)c5ccc(-c6ccccc6)cc5)cc4)ccc23)c2ccccc2-c2ccccc21 ; CC1(c2cccc3c2sc2ccc(-c4cccc(N(c5ccc(-c6ccccc6)cc5)c5ccc(-c6ccccc6)cc5)c4)cc23)c2ccccc2-c2ccccc21 ; CC1(c2cccc3c2sc2cc(-c4ccc(N(c5ccc(-c6ccccc6)cc5)c5cccc(-c6ccccc6)c5)cc4)ccc23)c2ccccc2-c2ccccc21 ; CC1(C)c2ccccc2-c2ccc(N(c3ccccc3)c3ccc(-c4ccc5c(sc6ccccc65)c4C4(C)c5ccccc5-c5ccccc54)cc3)cc21]

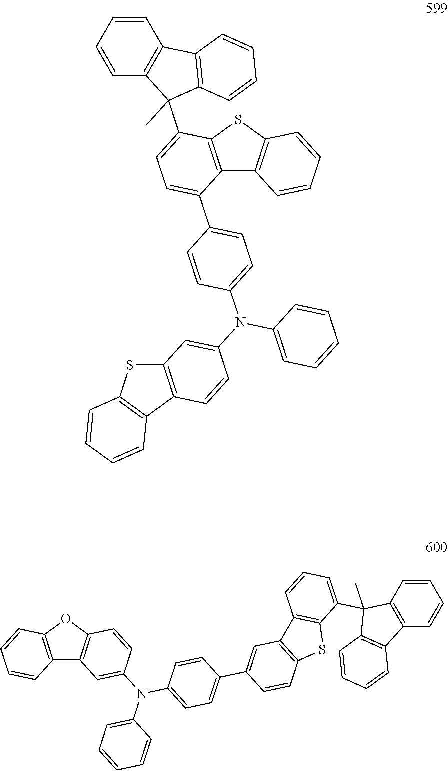 [SMILES: CC1(c2ccc(-c3ccc(N(c4ccccc4)c4ccc5c(c4)sc4ccccc45)cc3)c3c2sc2ccccc23)c2ccccc2-c2ccccc21 ; CC1(c2cccc3c2sc2ccc(-c4ccc(N(c5ccccc5)c5ccc6oc7ccccc7c6c5)cc4)cc23)c2ccccc2-c2ccccc21]

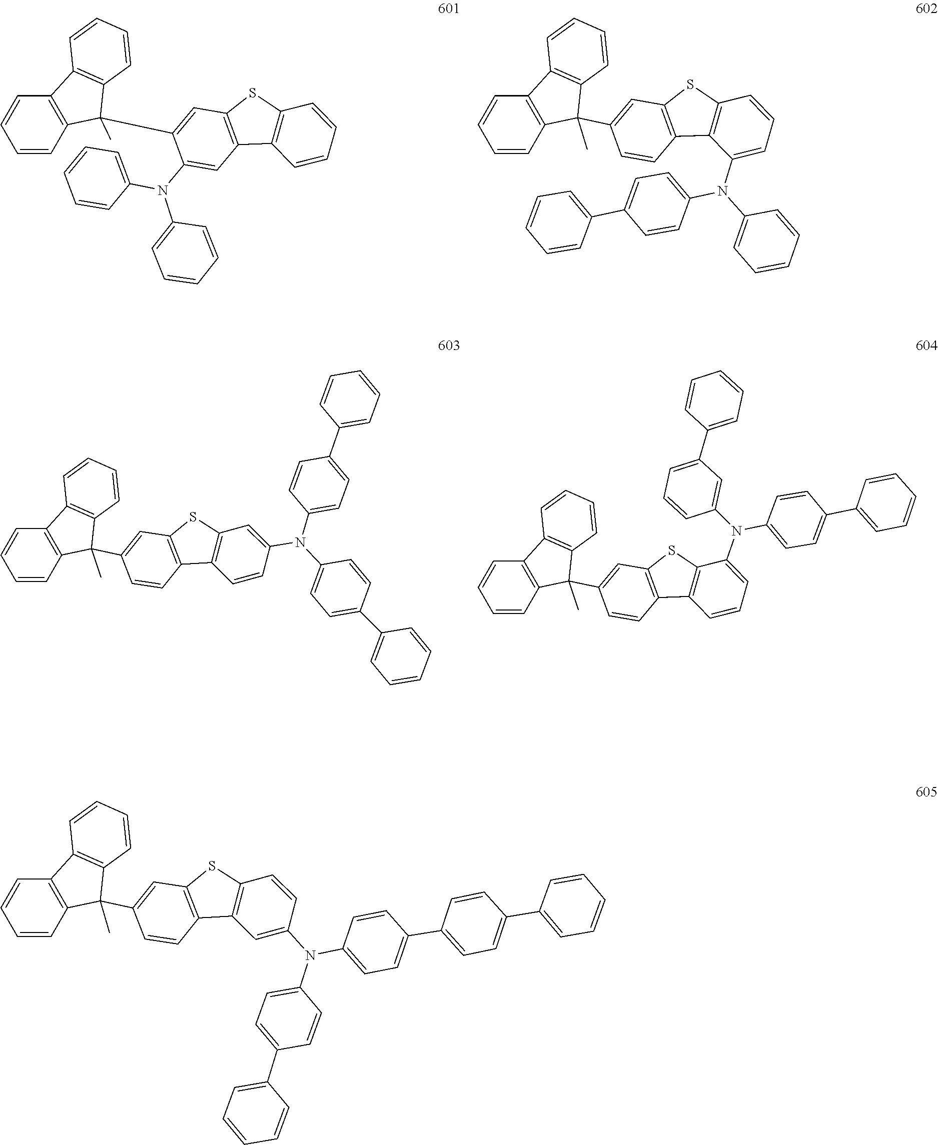 [SMILES: CC1(c2cc3sc4ccccc4c3cc2N(c2ccccc2)c2ccccc2)c2ccccc2-c2ccccc21 ; CC1(c2ccc3c(c2)sc2cccc(N(c4ccccc4)c4ccc(-c5ccccc5)cc4)c23)c2ccccc2-c2ccccc21 ; CC1(c2ccc3c(c2)sc2cc(N(c4ccc(-c5ccccc5)cc4)c4ccc(-c5ccccc5)cc4)ccc23)c2ccccc2-c2ccccc21 ; CC1(c2ccc3c(c2)sc2c(N(c4ccc(-c5ccccc5)cc4)c4cccc(-c5ccccc5)c4)cccc23)c2ccccc2-c2ccccc21 ; CC1(c2ccc3c(c2)sc2ccc(N(c4ccc(-c5ccccc5)cc4)c4ccc(-c5ccc(-c6ccccc6)cc5)cc4)cc23)c2ccccc2-c2ccccc21]

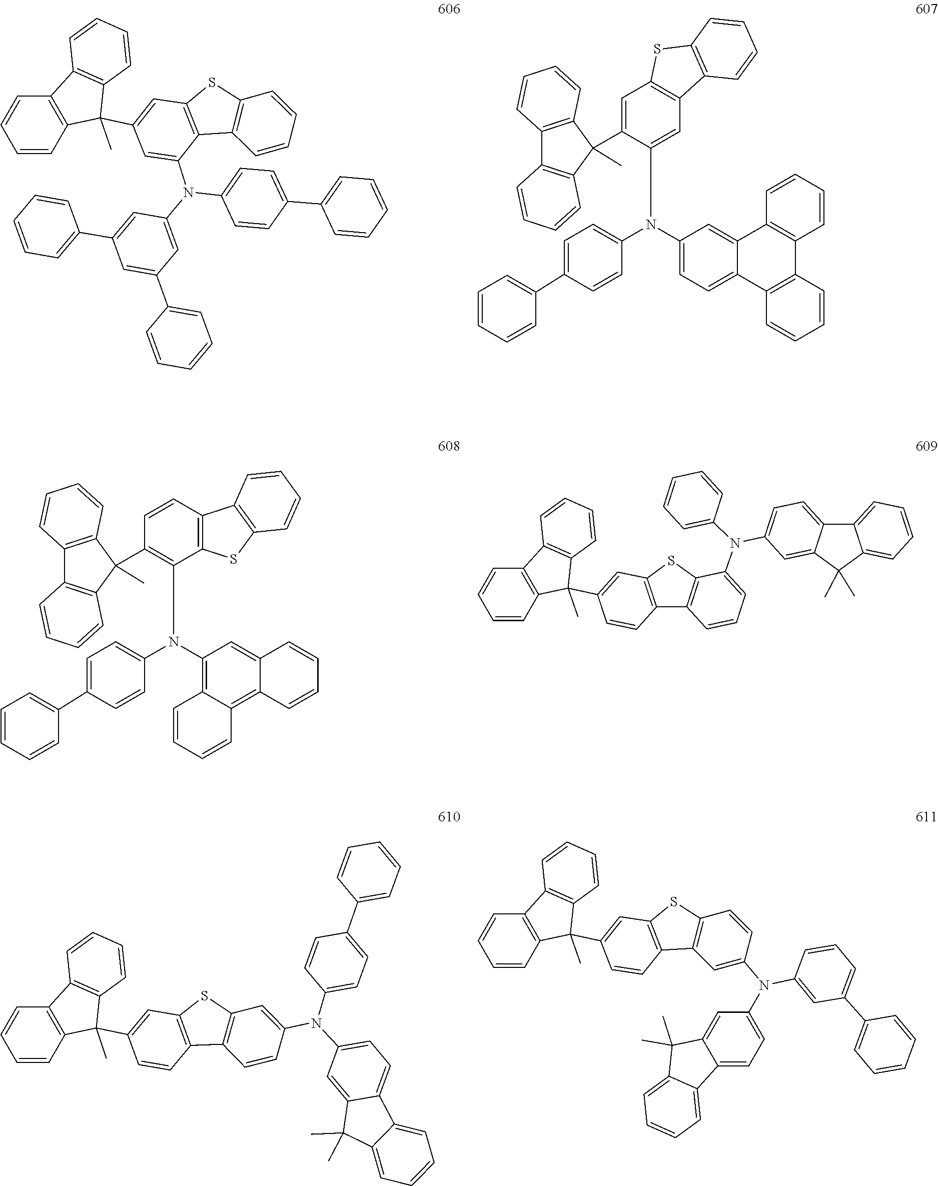

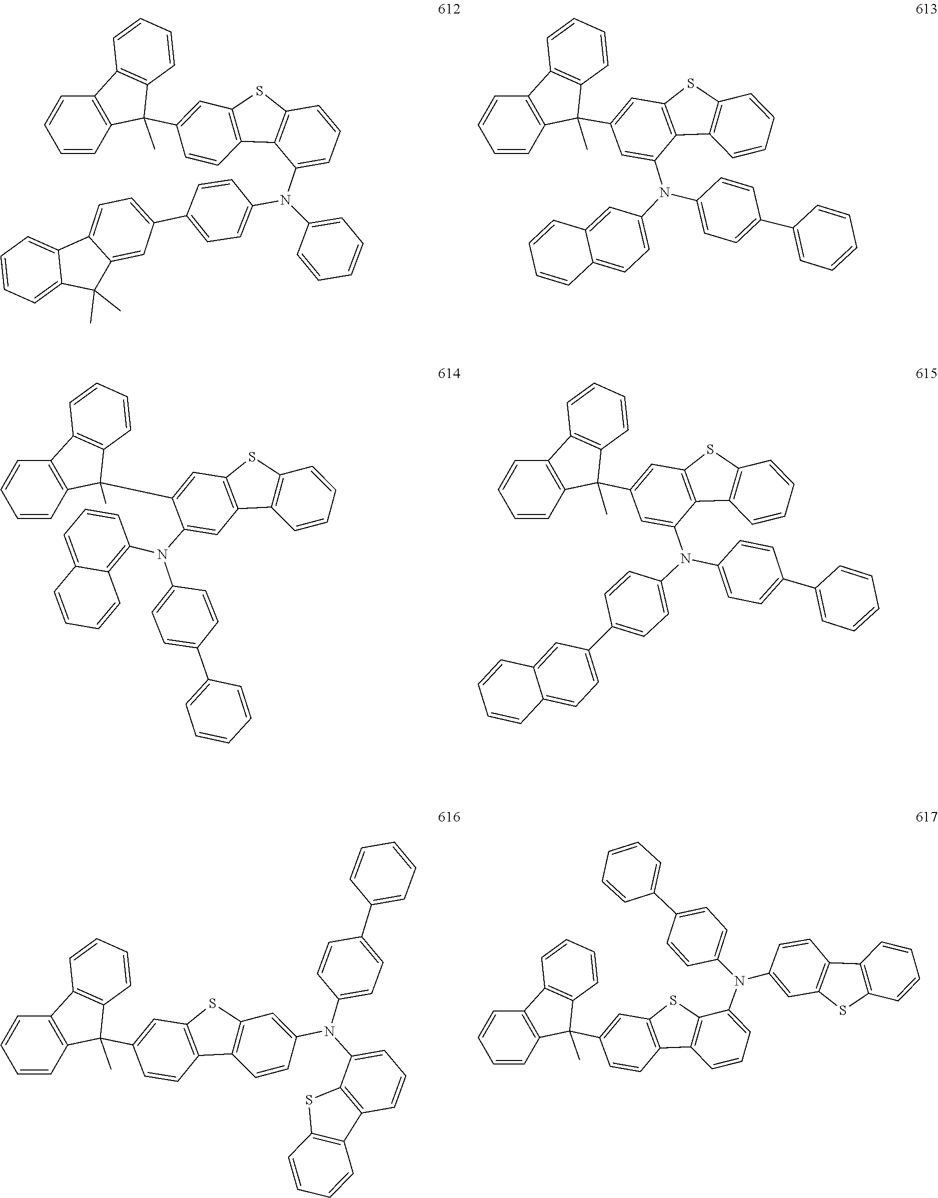

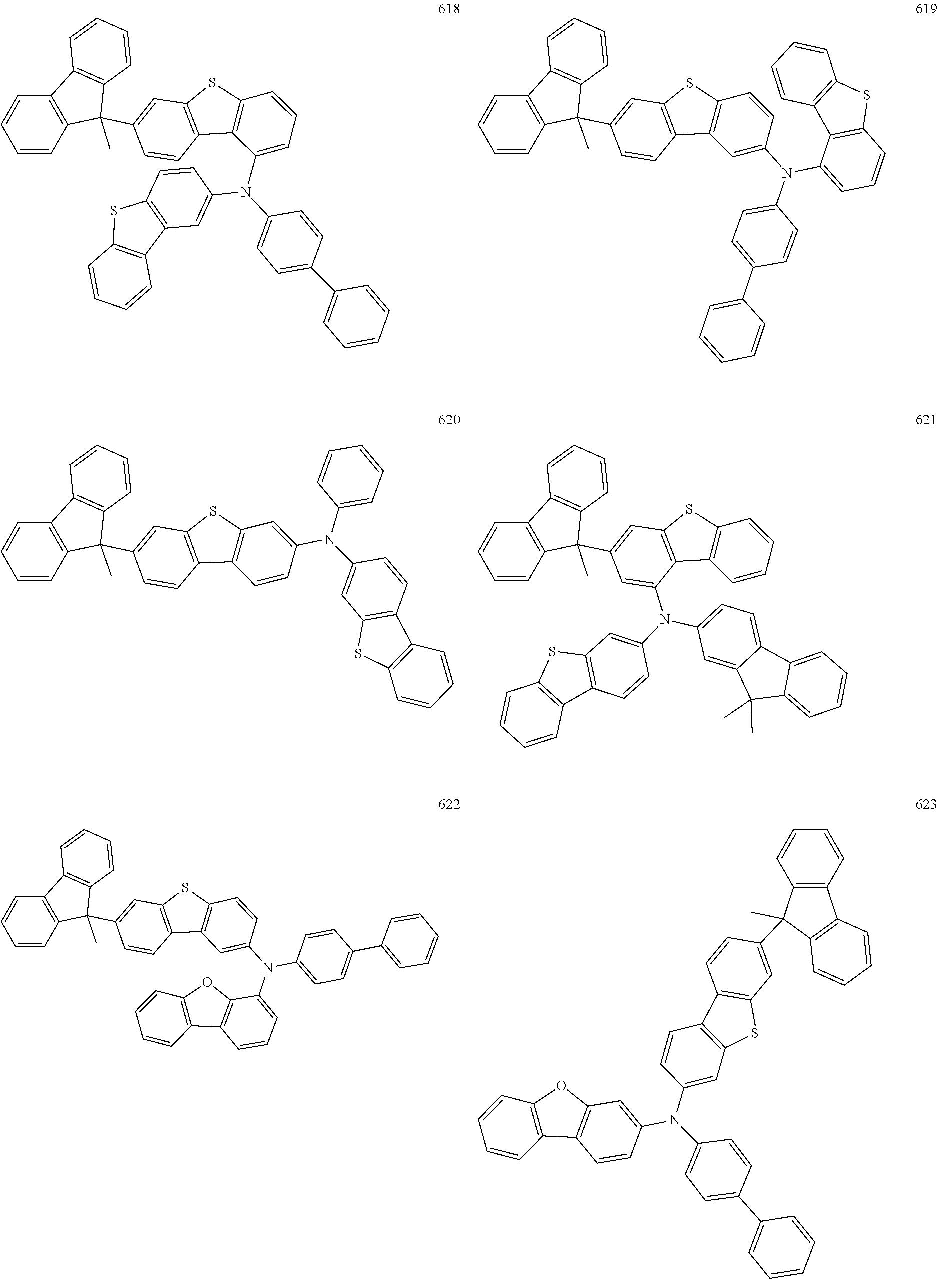

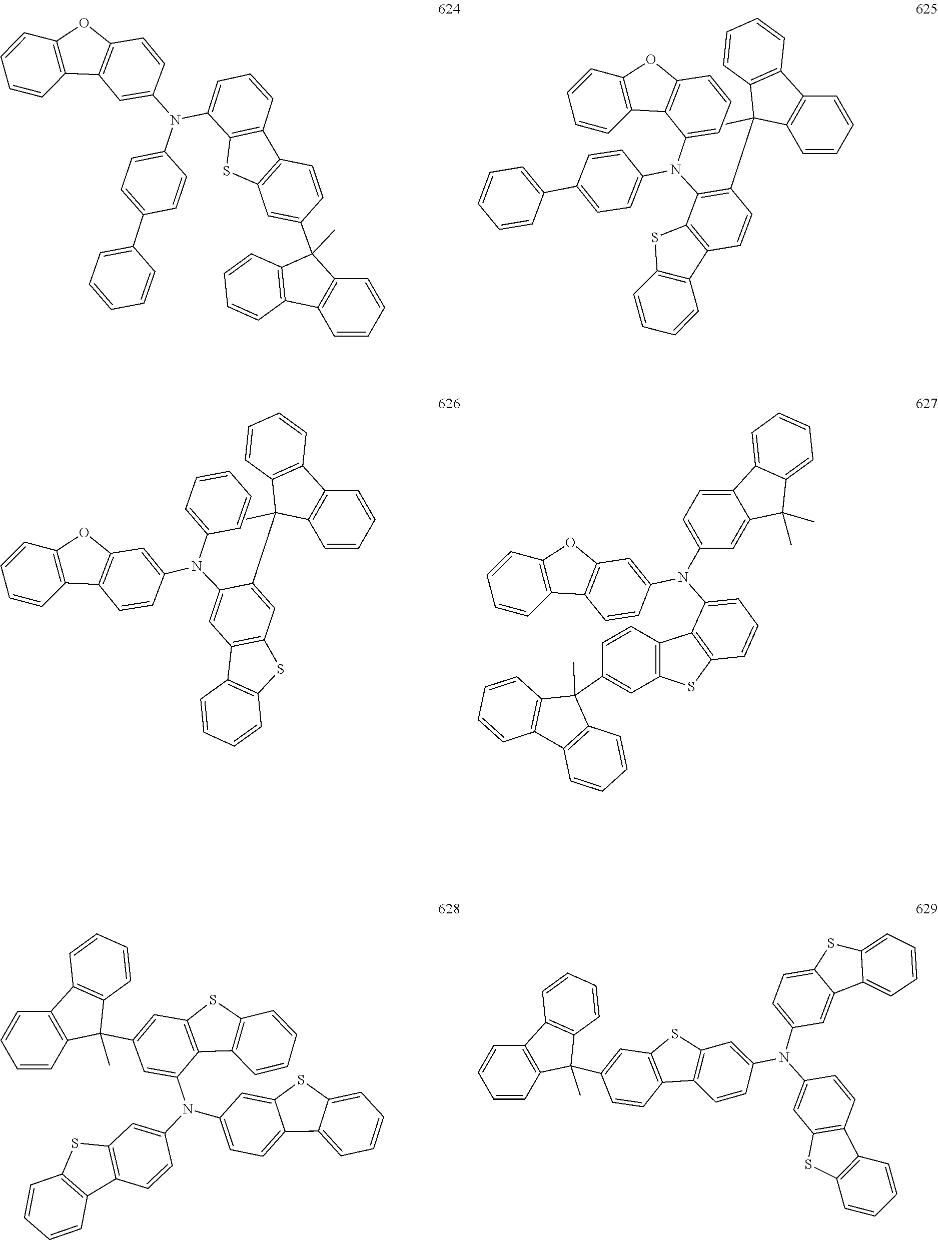

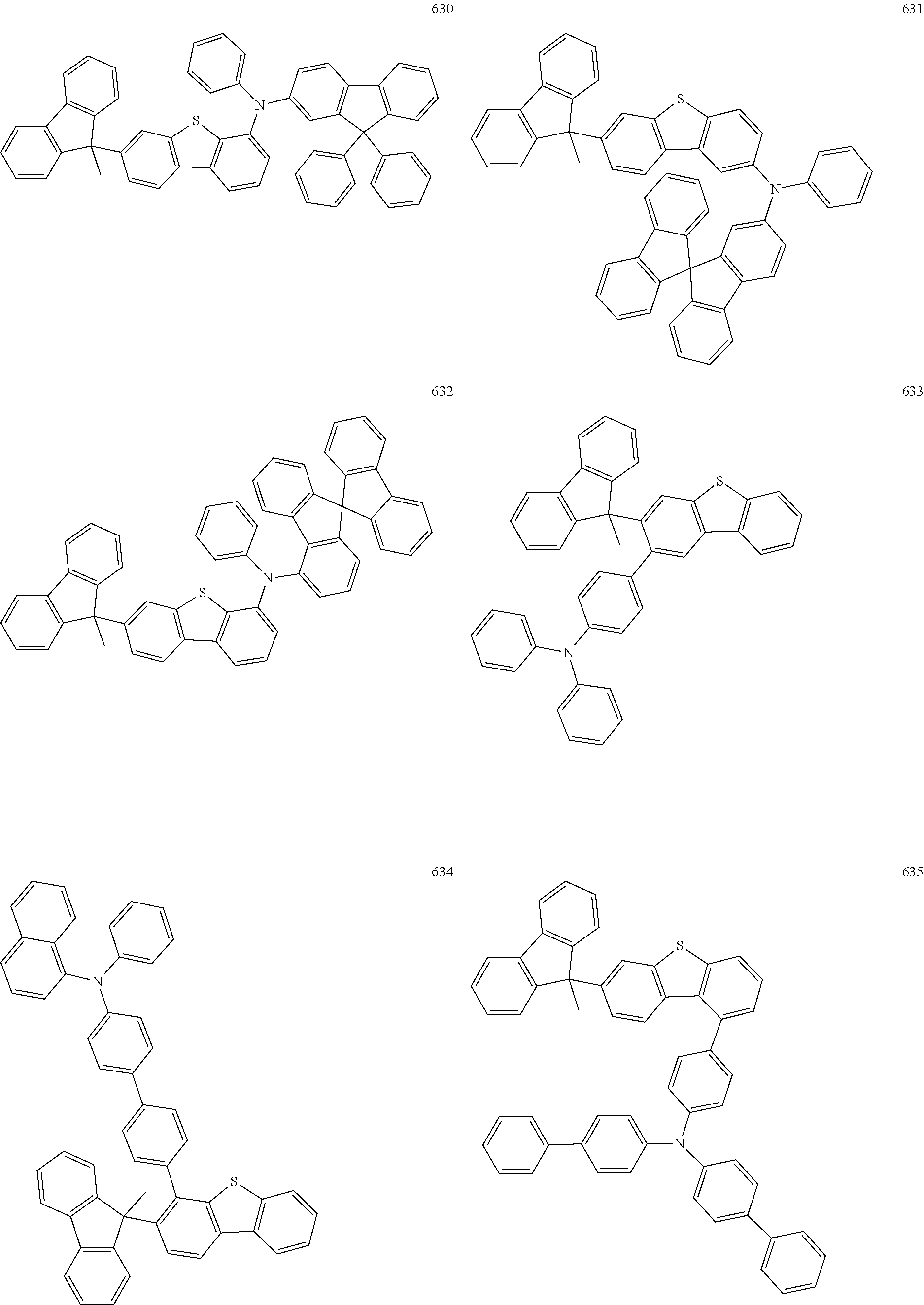

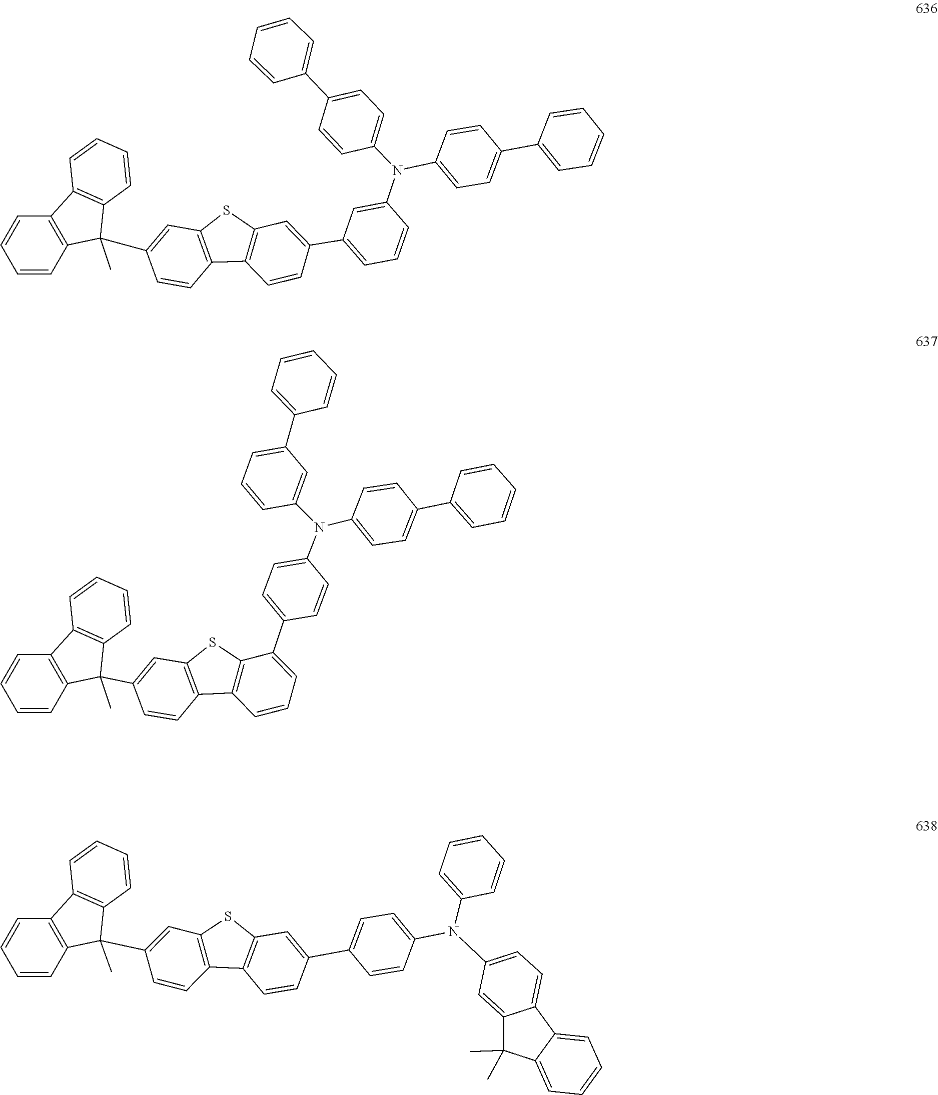 [SMILES: CC1(c2ccc3c(c2)sc2cc(-c4cccc(N(c5ccc(-c6ccccc6)cc5)c5ccc(-c6ccccc6)cc5)c4)ccc23)c2ccccc2-c2ccccc21 ; CC1(c2ccc3c(c2)sc2c(-c4ccc(N(c5ccc(-c6ccccc6)cc5)c5cccc(-c6ccccc6)c5)cc4)cccc23)c2ccccc2-c2ccccc21 ; CC1(C)c2ccccc2-c2ccc(N(c3ccccc3)c3ccc(-c4ccc5c(c4)sc4cc(C6(C)c7ccccc7-c7ccccc76)ccc45)cc3)cc21]

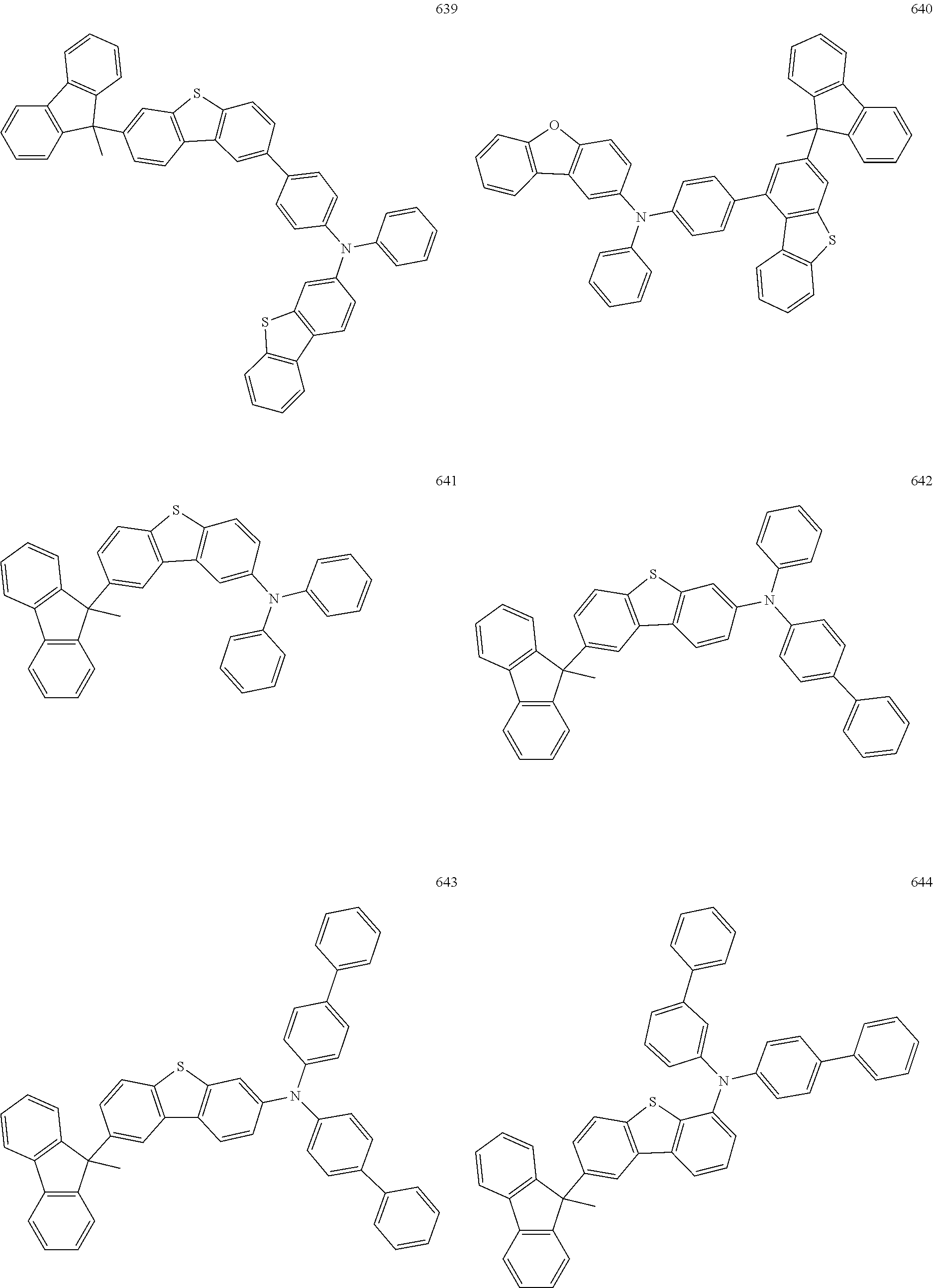 [SMILES: CC1(c2ccc3c(c2)sc2ccc(-c4ccc(N(c5ccccc5)c5ccc6c(c5)sc5ccccc56)cc4)cc23)c2ccccc2-c2ccccc21 ; CC1(c2cc(-c3ccc(N(c4ccccc4)c4ccc5oc6ccccc6c5c4)cc3)c3c(c2)sc2ccccc23)c2ccccc2-c2ccccc21 ; CC1(c2ccc3sc4ccc(N(c5ccccc5)c5ccccc5)cc4c3c2)c2ccccc2-c2ccccc21 ; CC1(c2ccc3sc4cc(N(c5ccccc5)c5ccc(-c6ccccc6)cc5)ccc4c3c2)c2ccccc2-c2ccccc21 ; CC1(c2ccc3sc4cc(N(c5ccc(-c6ccccc6)cc5)c5ccc(-c6ccccc6)cc5)ccc4c3c2)c2ccccc2-c2ccccc21 ; CC1(c2ccc3sc4c(N(c5ccc(-c6ccccc6)cc5)c5cccc(-c6ccccc6)c5)cccc4c3c2)c2ccccc2-c2ccccc21]

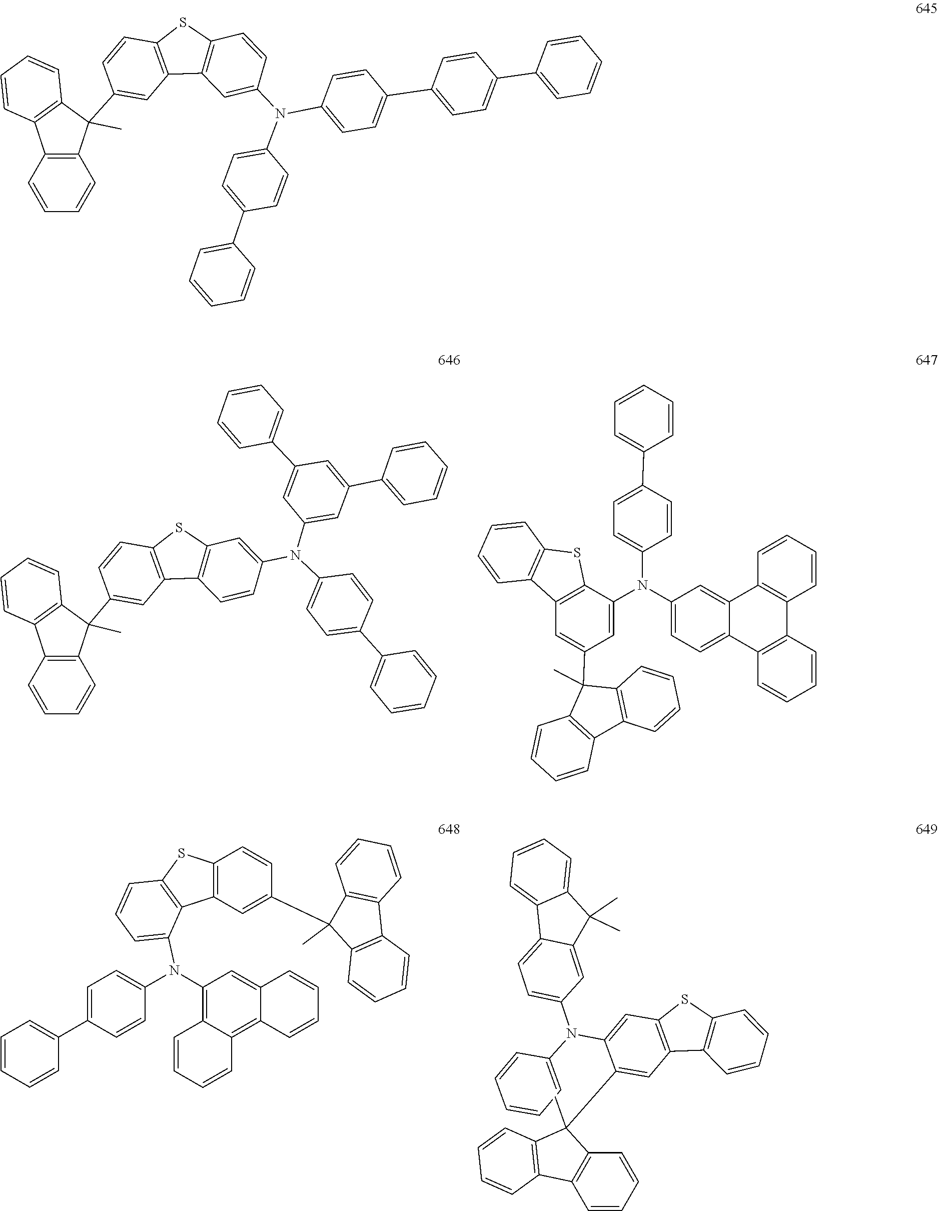

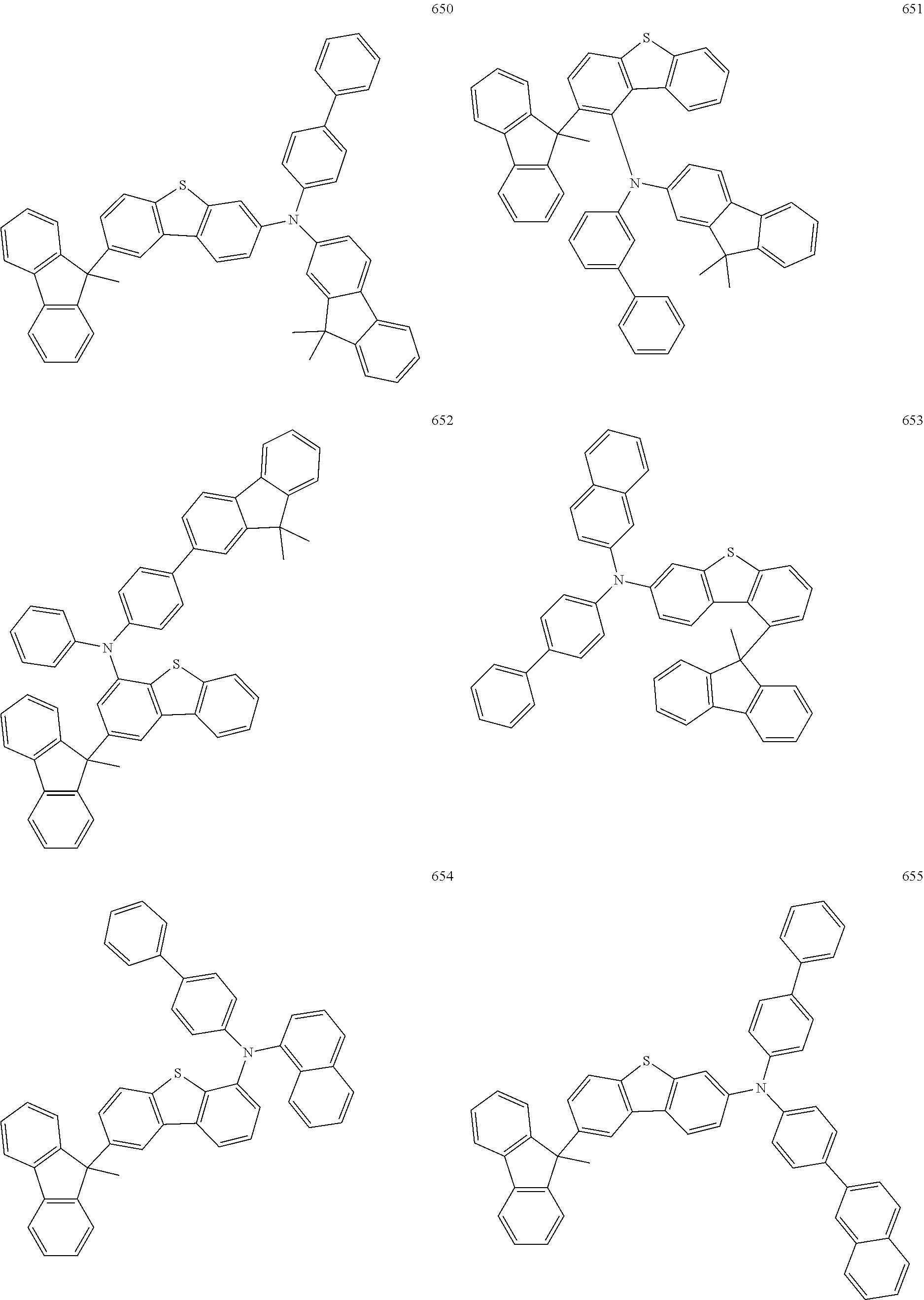

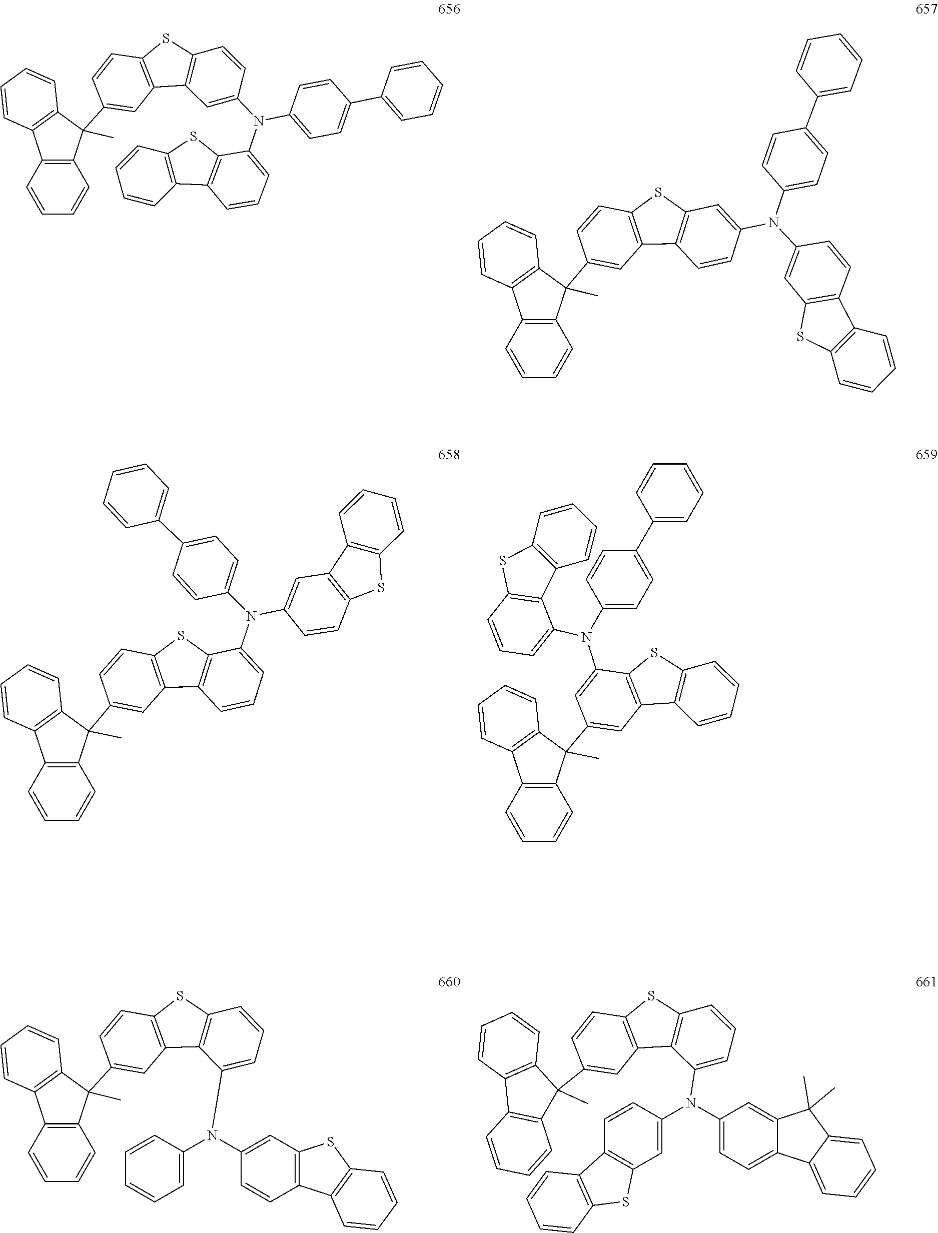

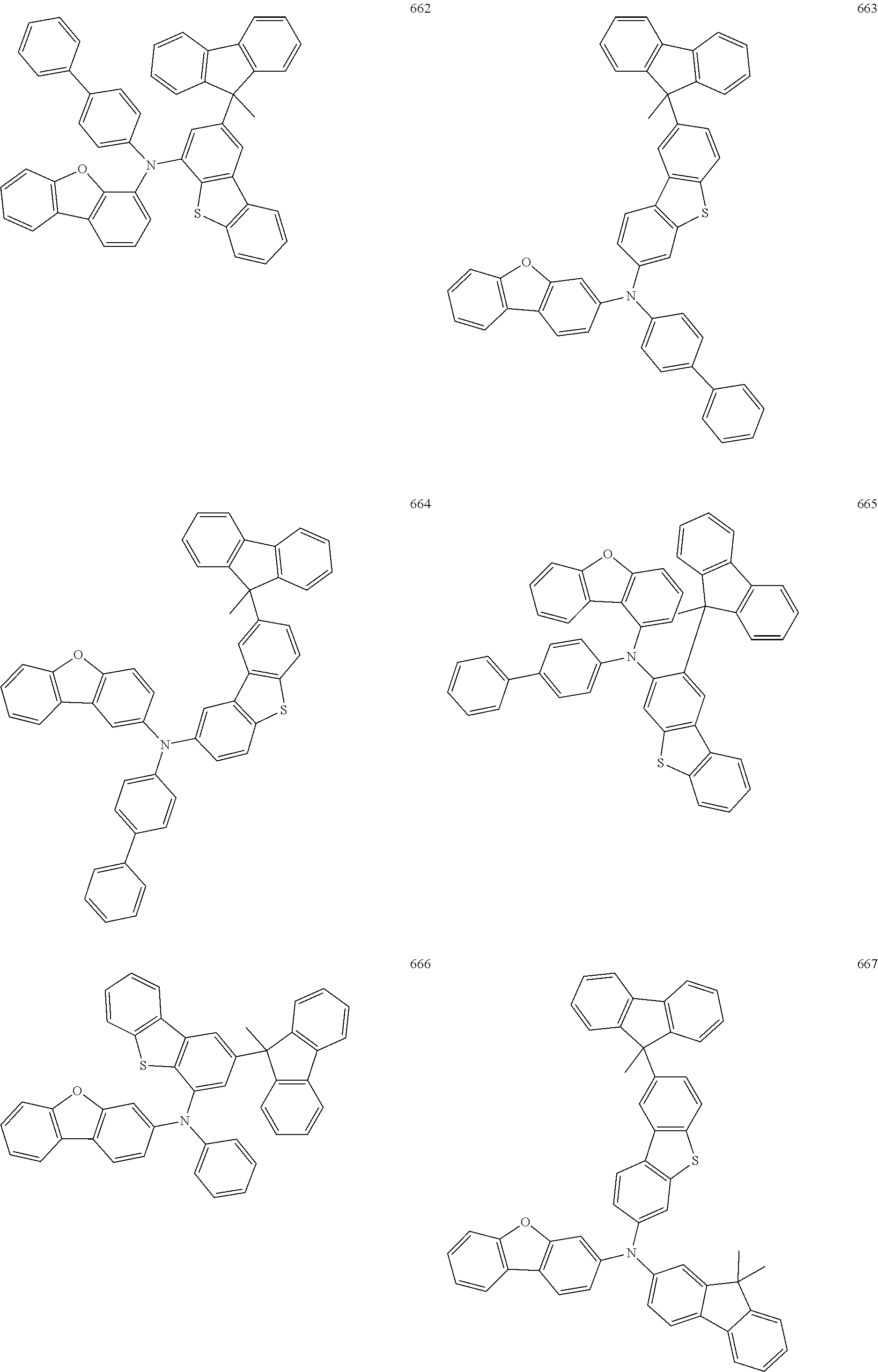

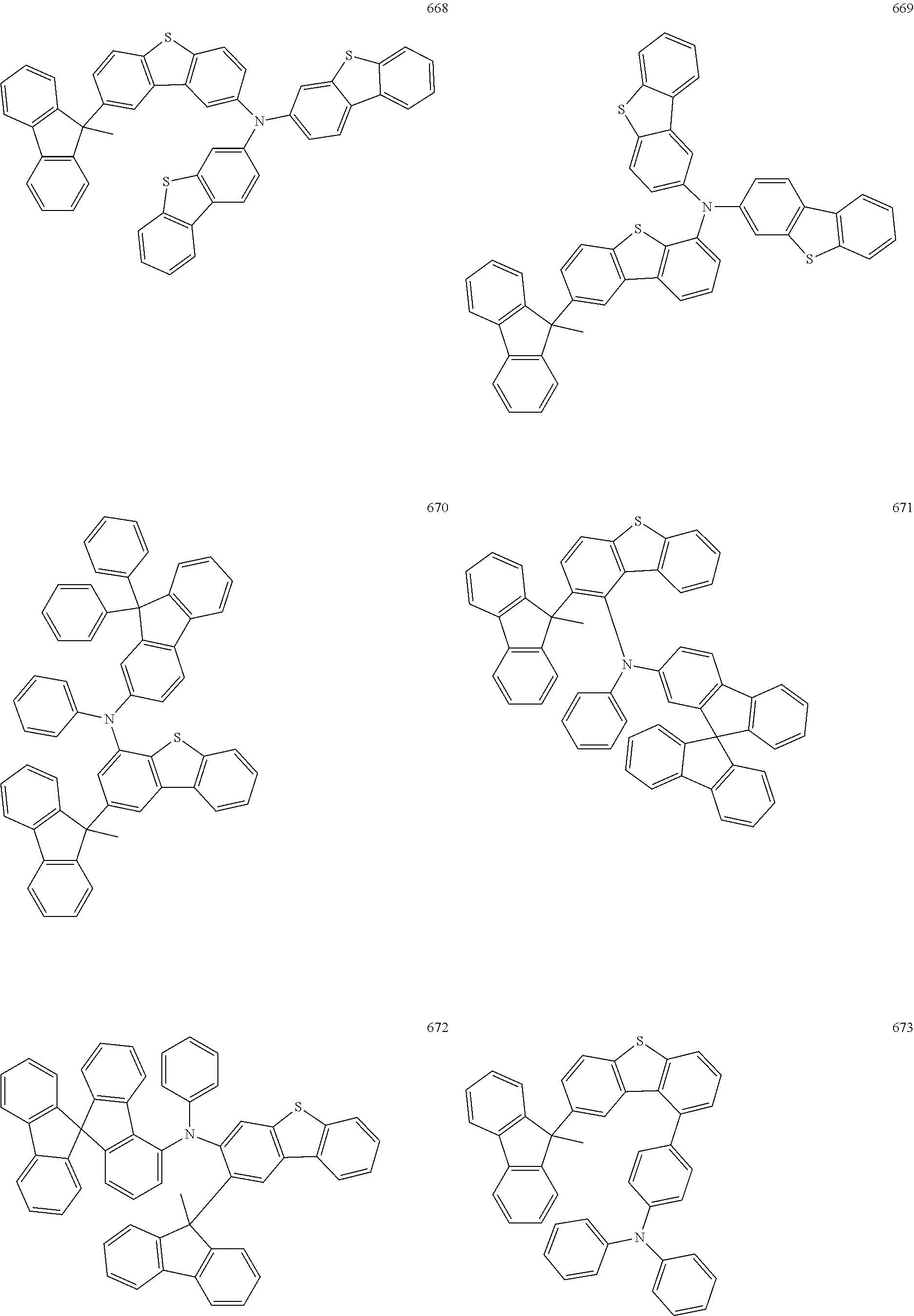

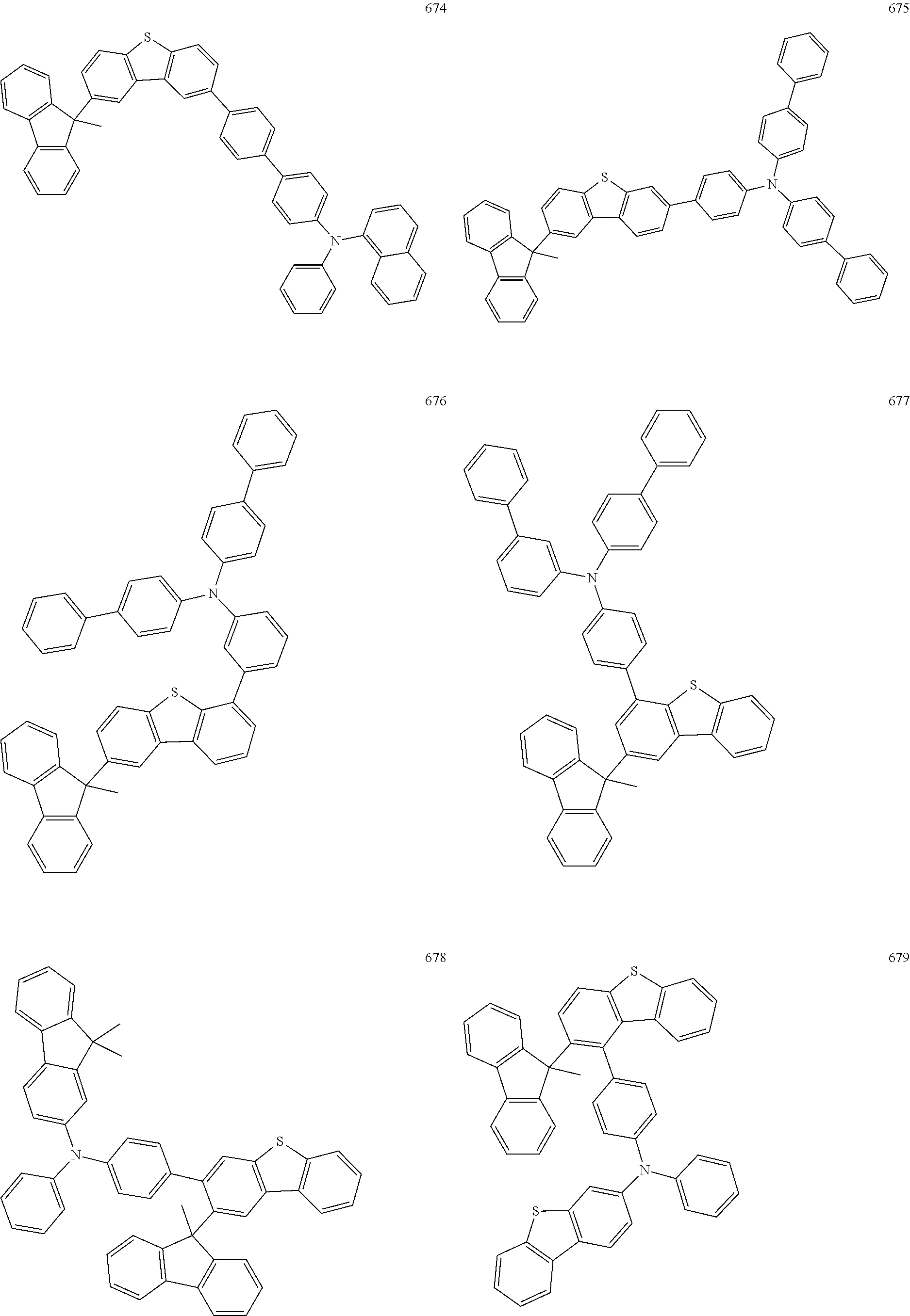

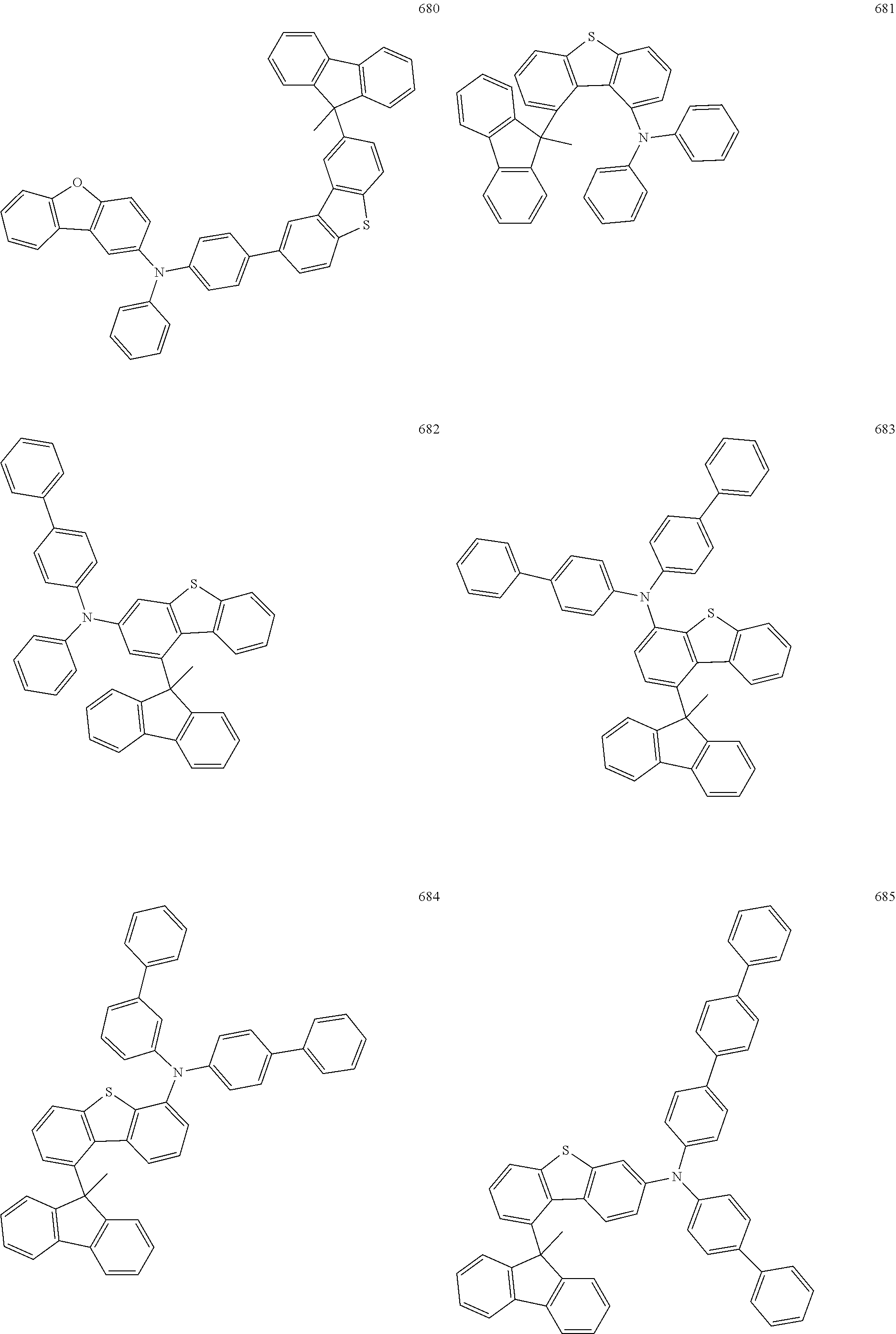

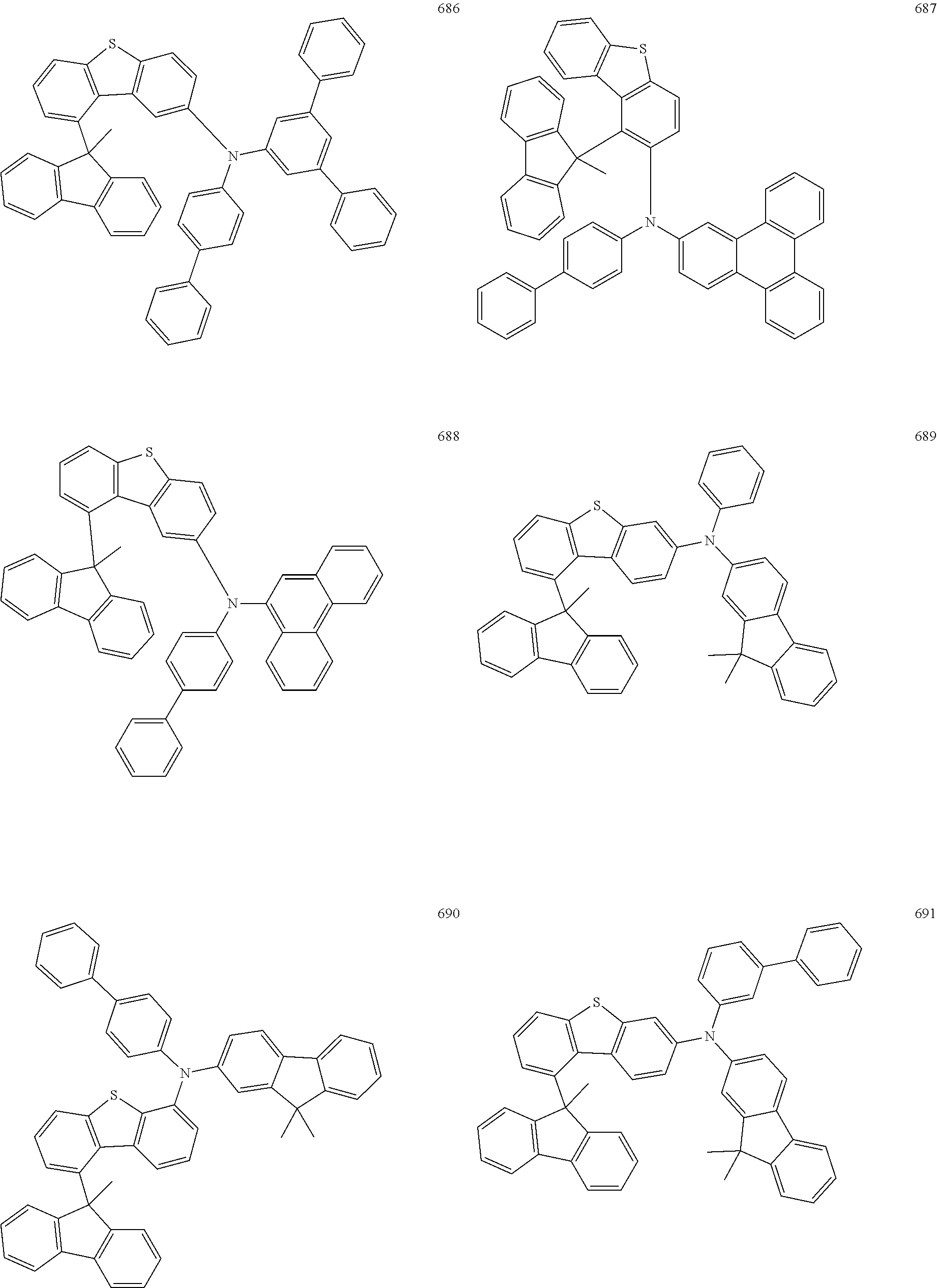

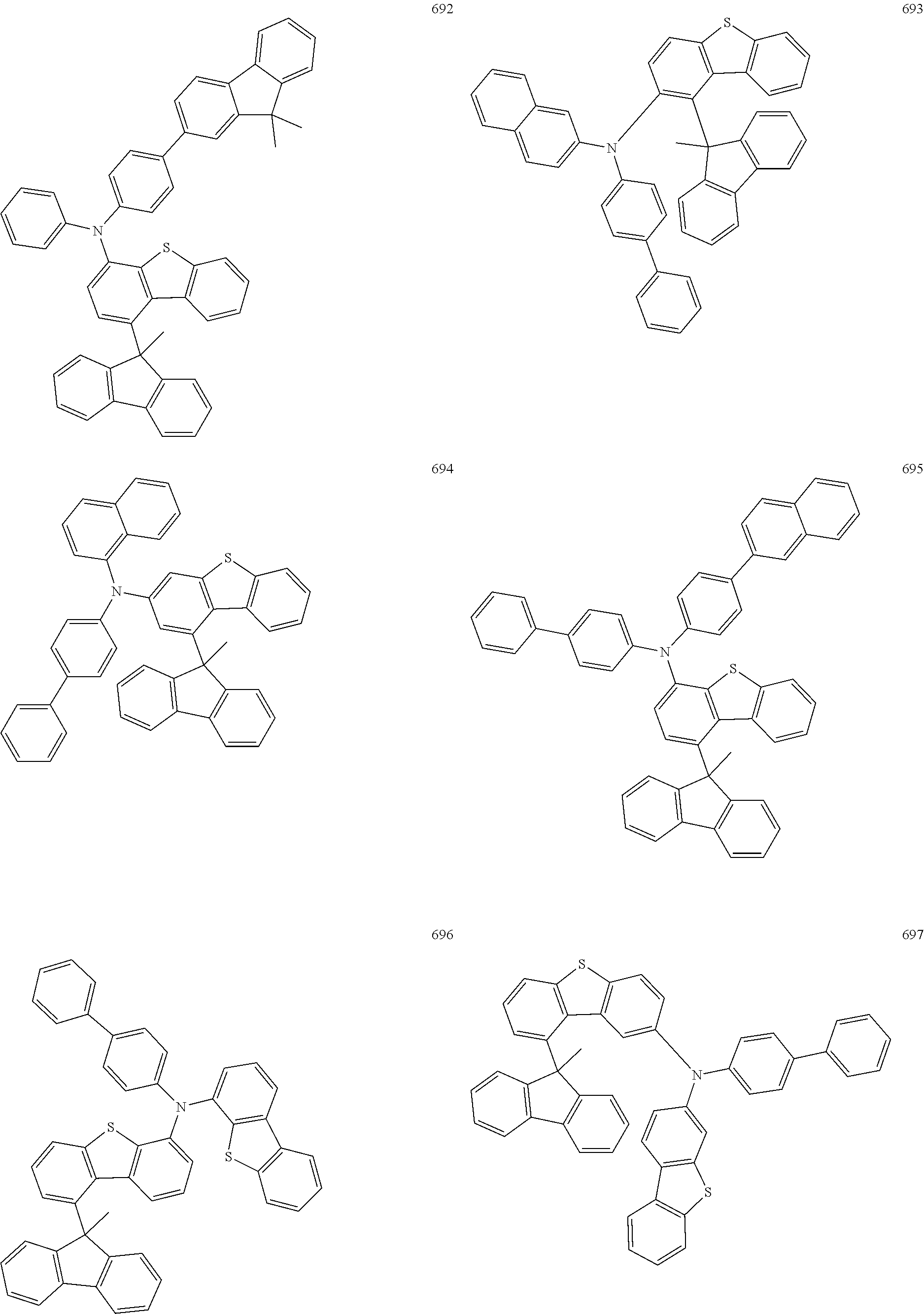

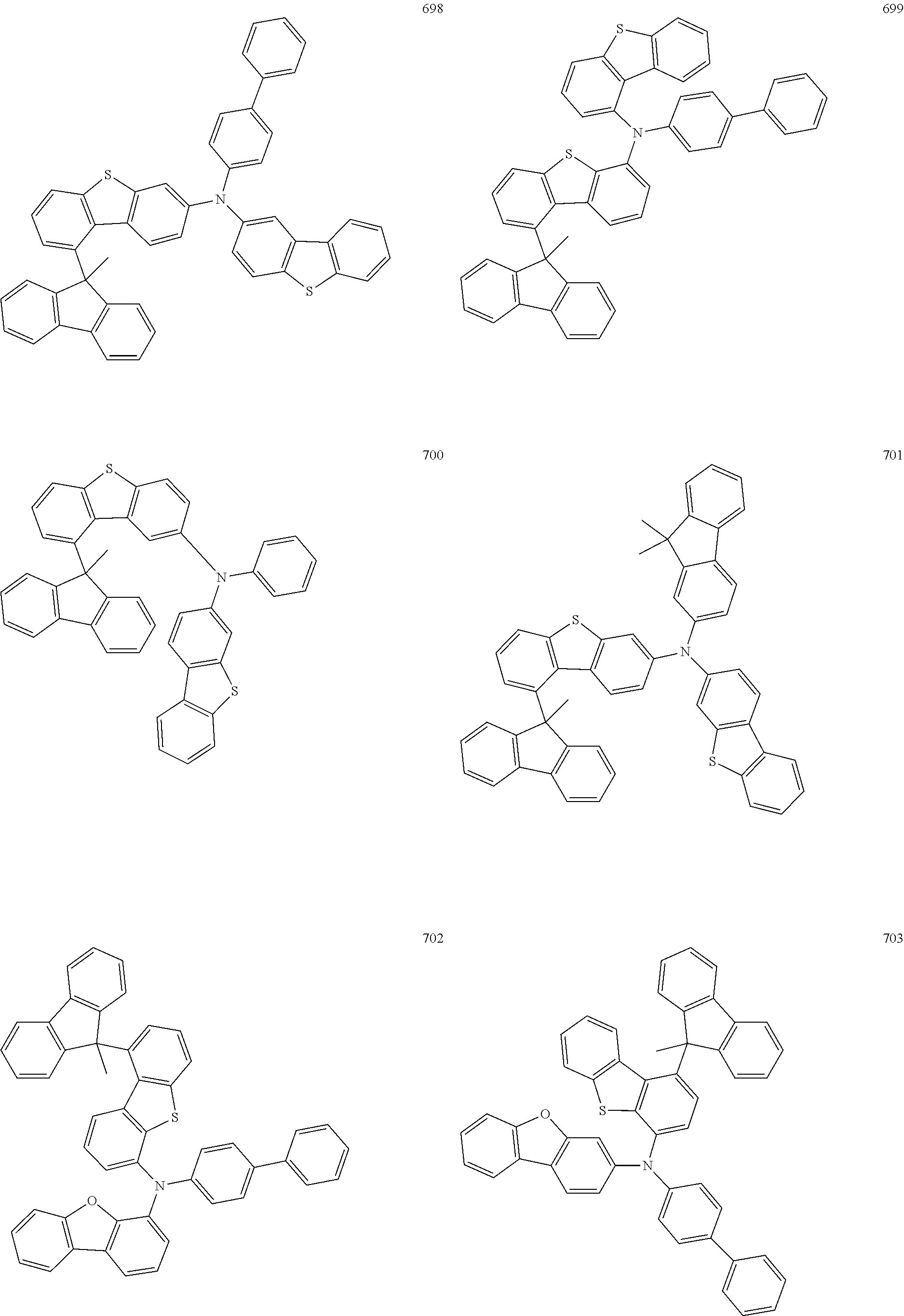

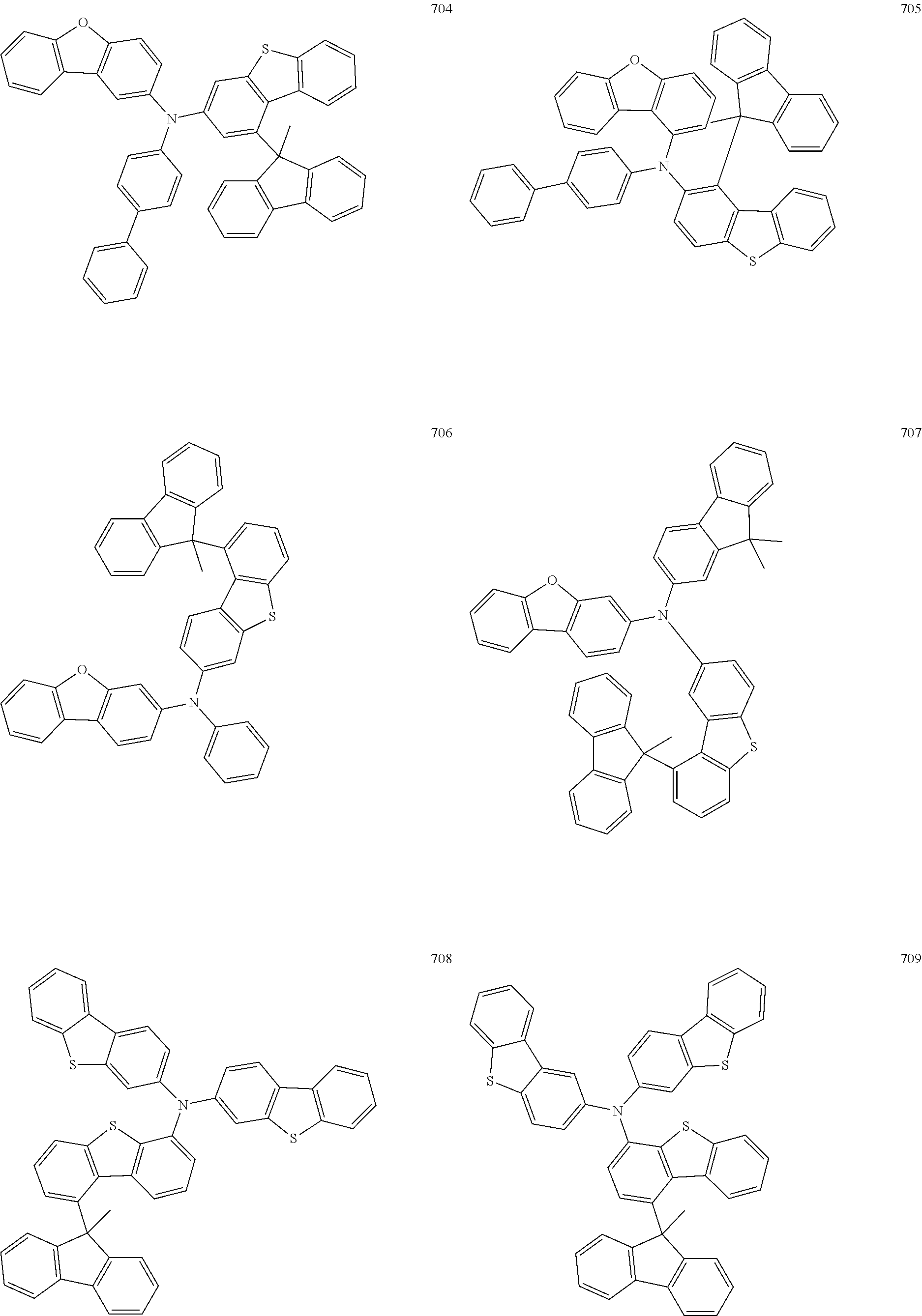

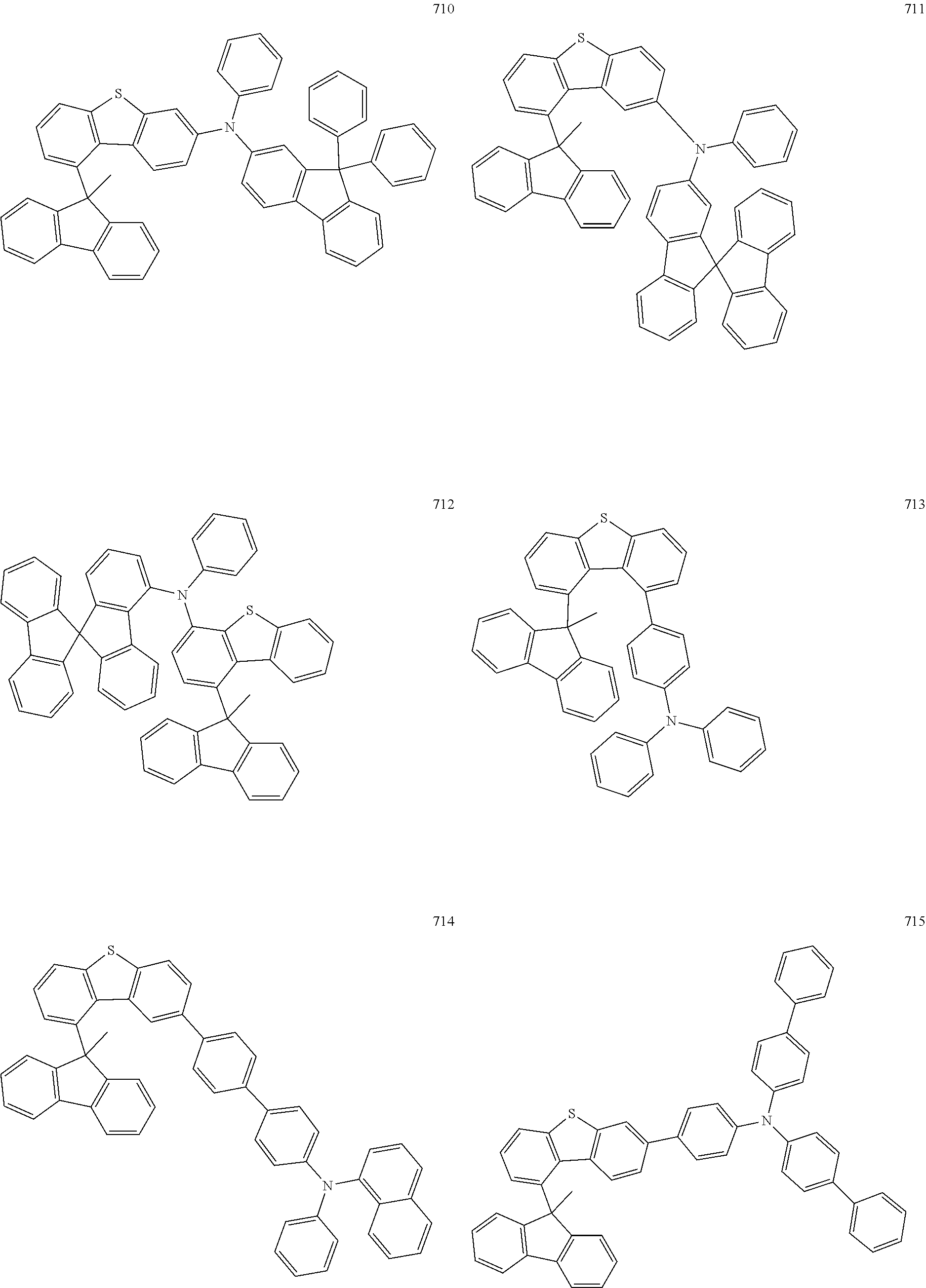

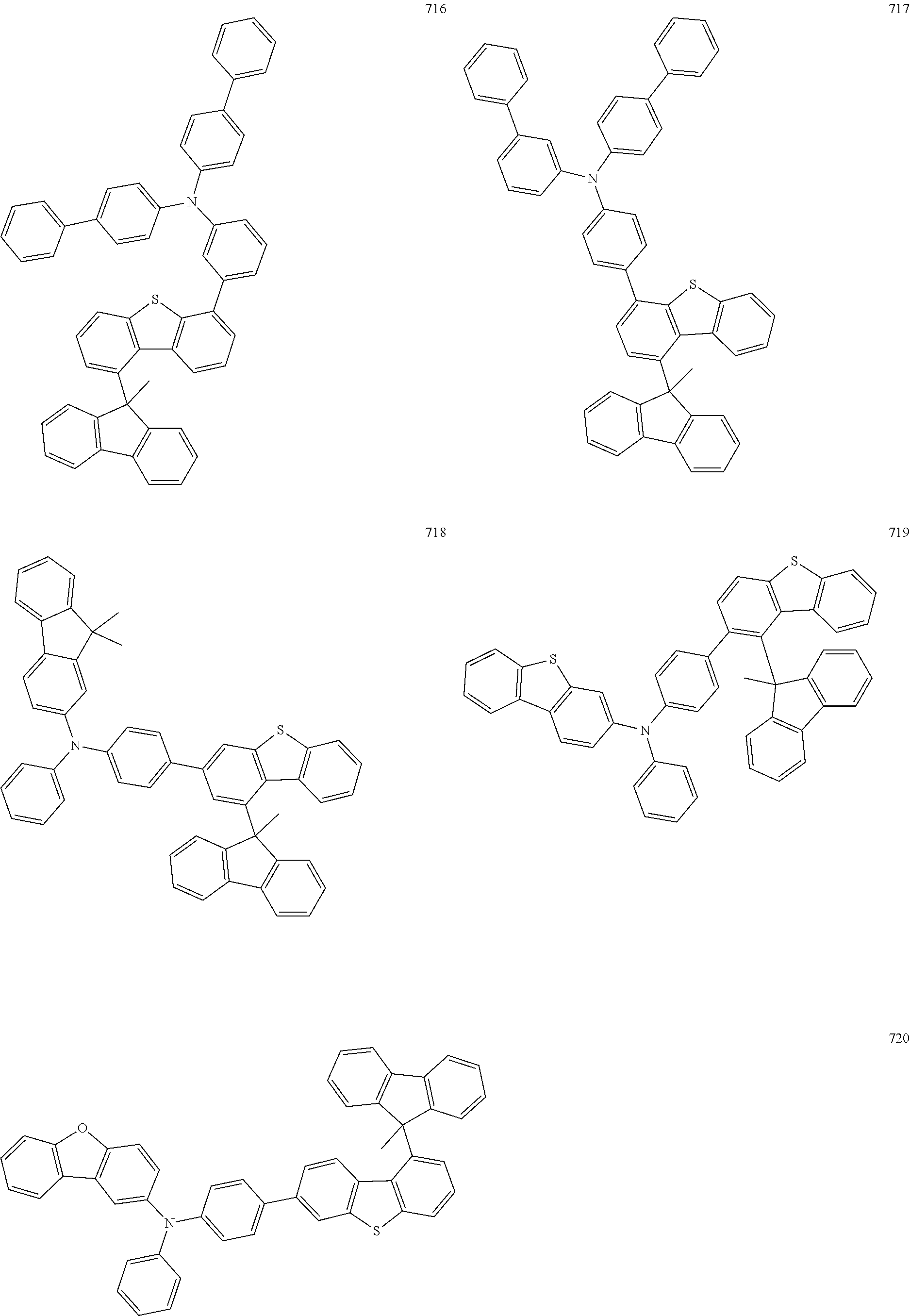 [SMILES: CC1(c2cccc3sc4c(-c5cccc(N(c6ccc(-c7ccccc7)cc6)c6ccc(-c7ccccc7)cc6)c5)cccc4c23)c2ccccc2-c2ccccc21 ; CC1(c2ccc(-c3ccc(N(c4ccc(-c5ccccc5)cc4)c4cccc(-c5ccccc5)c4)cc3)c3sc4ccccc4c23)c2ccccc2-c2ccccc21 ; CC1(C)c2ccccc2-c2ccc(N(c3ccccc3)c3ccc(-c4cc(C5(C)c6ccccc6-c6ccccc65)c5c(c4)sc4ccccc45)cc3)cc21 ; CC1(c2c(-c3ccc(N(c4ccccc4)c4ccc5c(c4)sc4ccccc45)cc3)ccc3sc4ccccc4c23)c2ccccc2-c2ccccc21 ; CC1(c2cccc3sc4cc(-c5ccc(N(c6ccccc6)c6ccc7oc8ccccc8c7c6)cc5)ccc4c23)c2ccccc2-c2ccccc21]

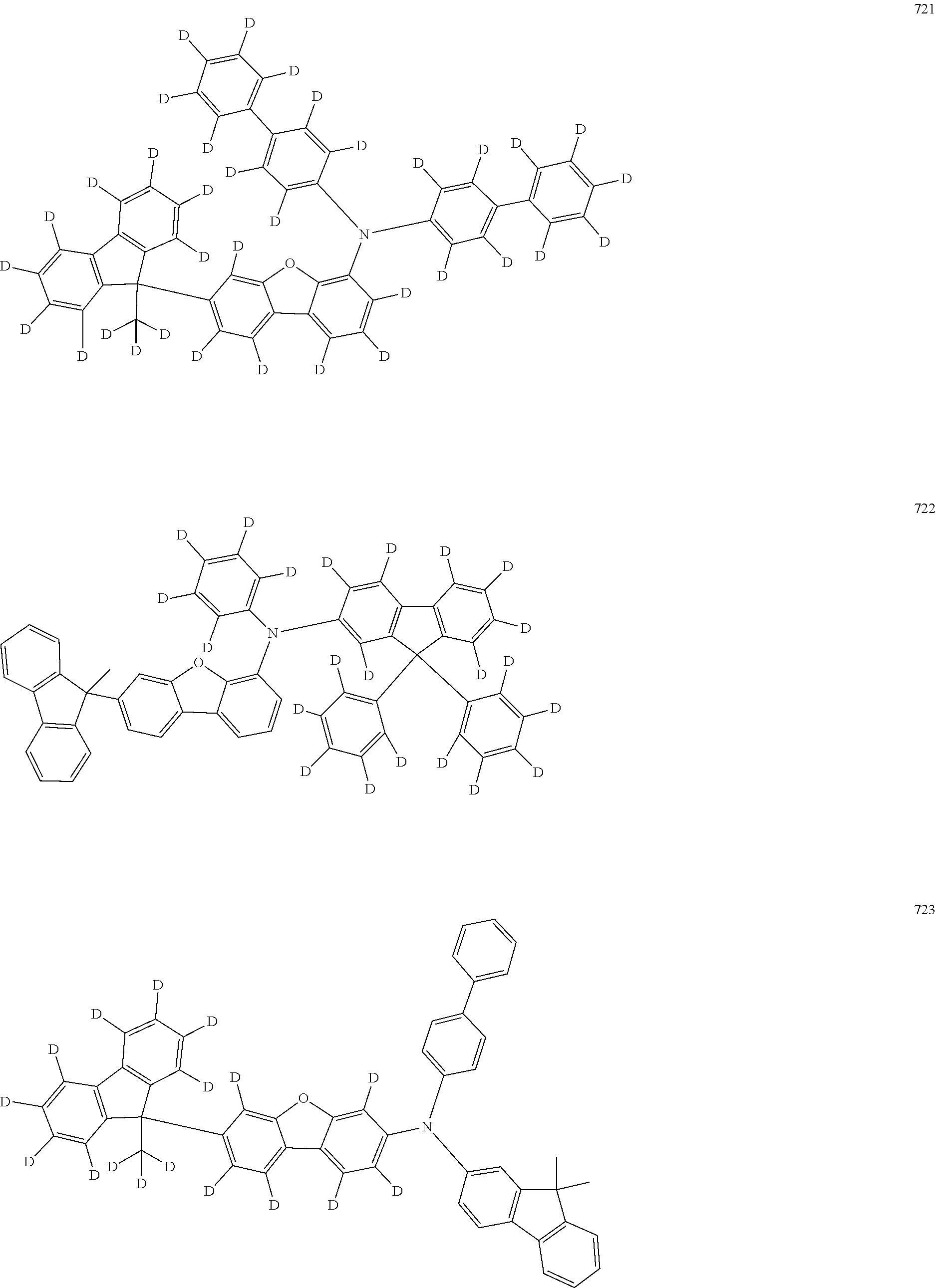

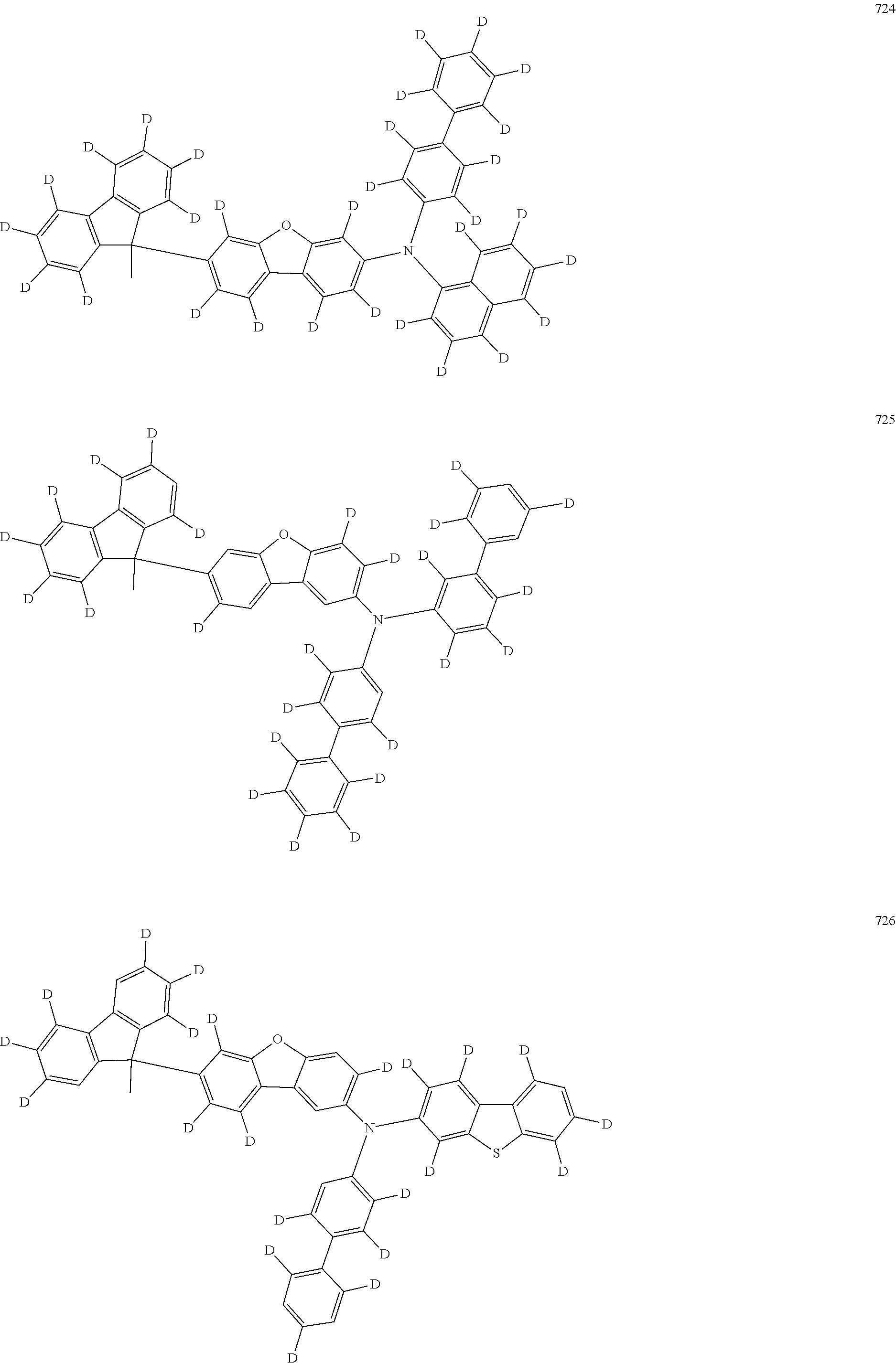

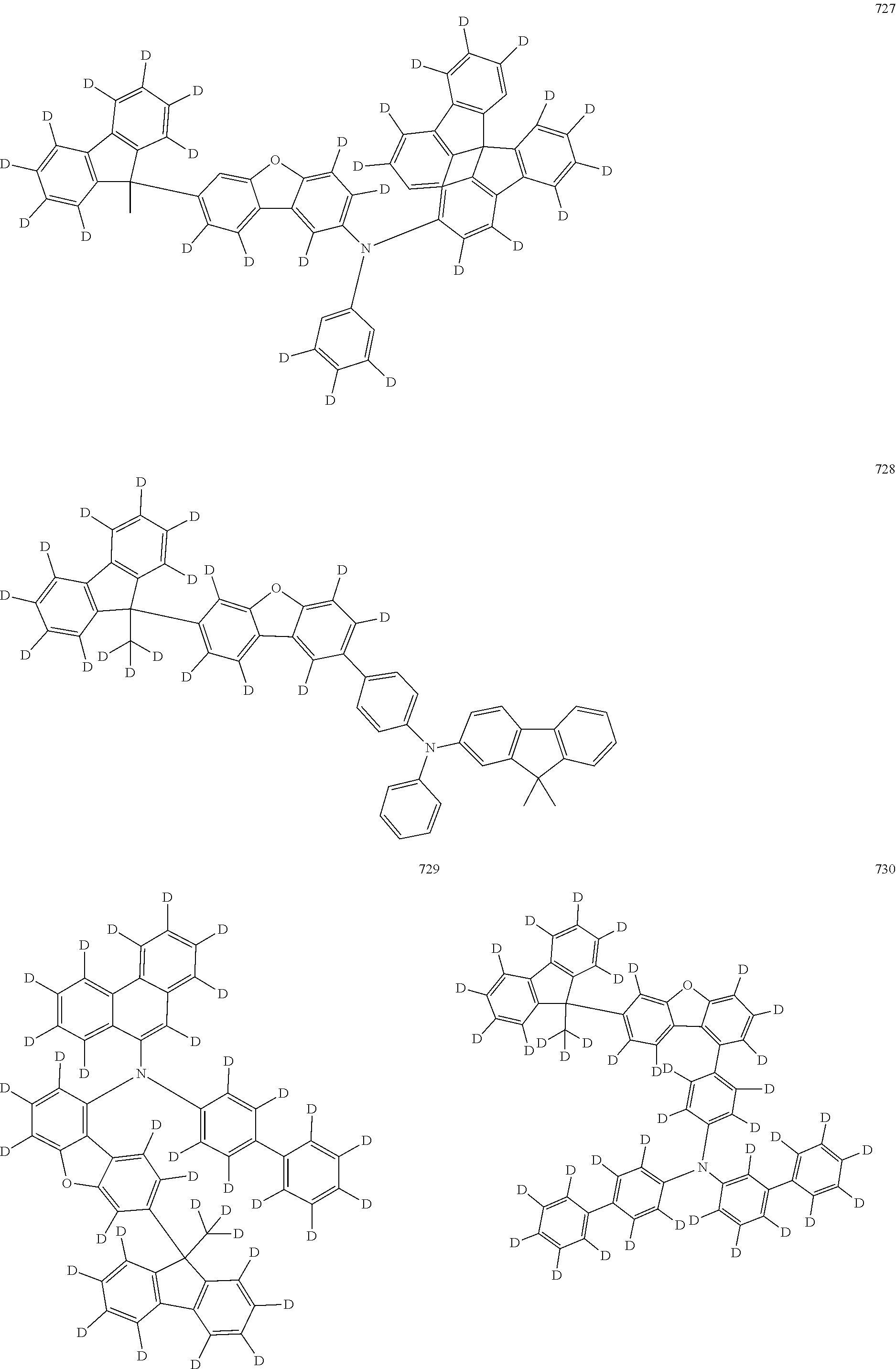

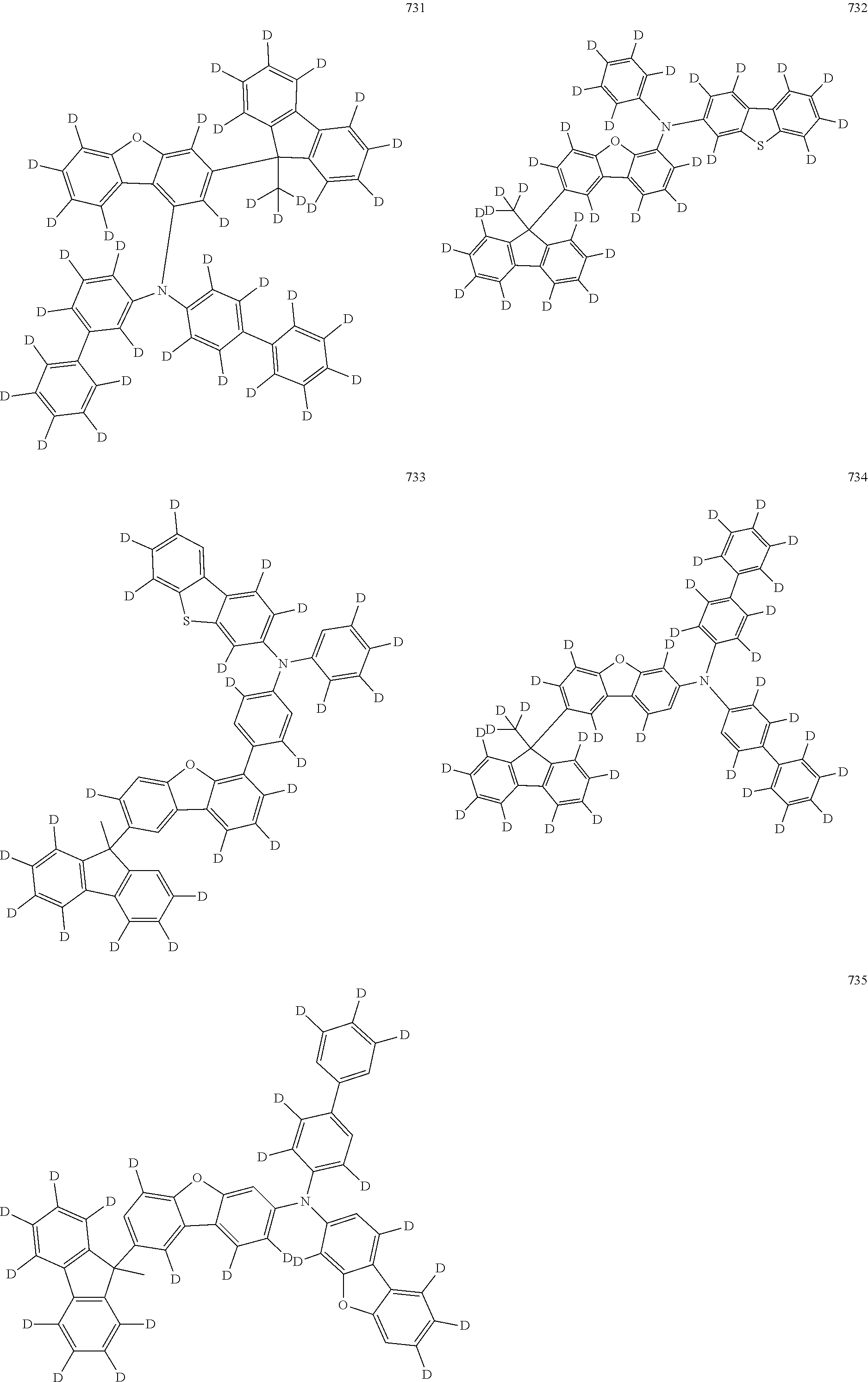

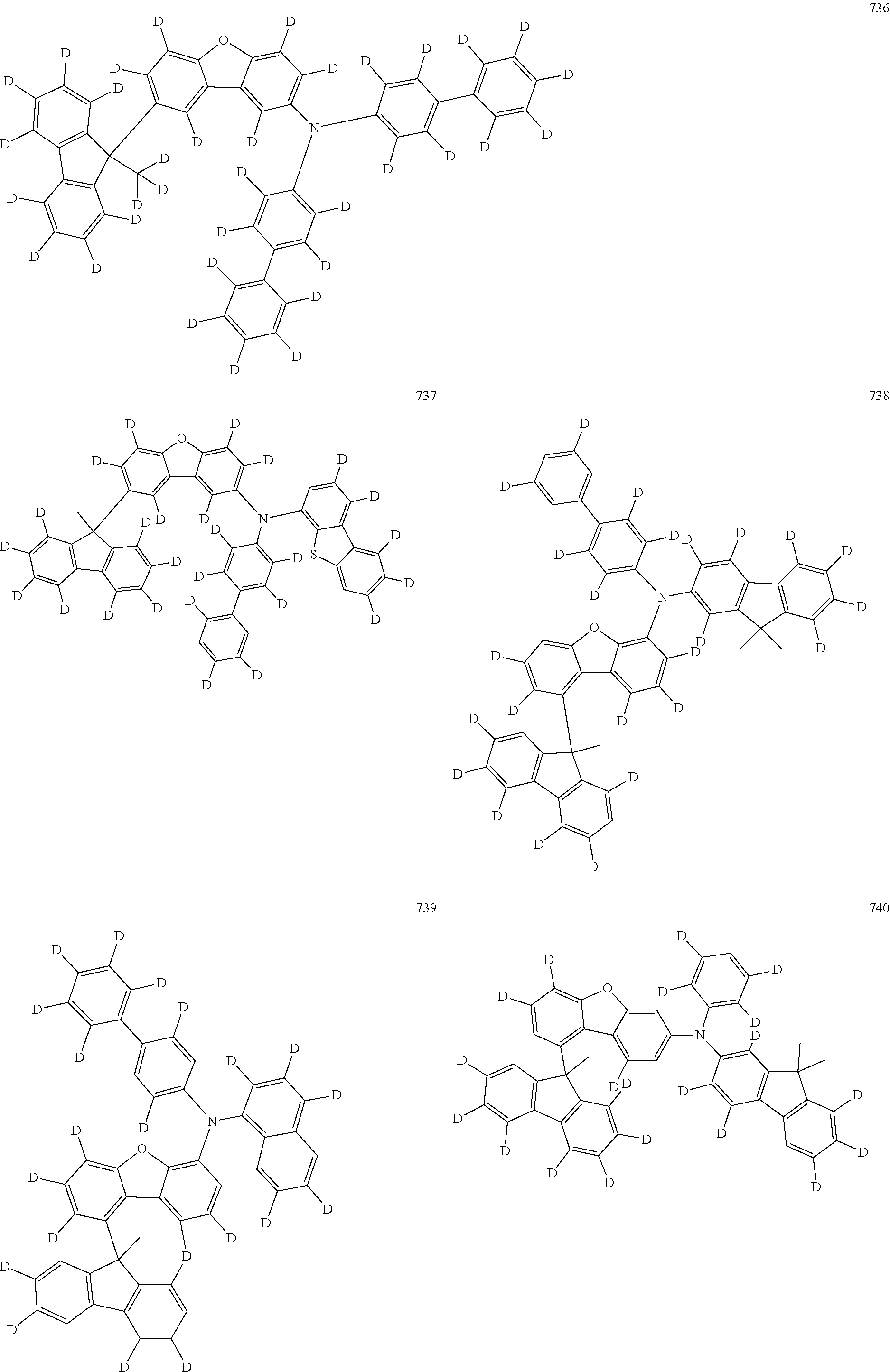

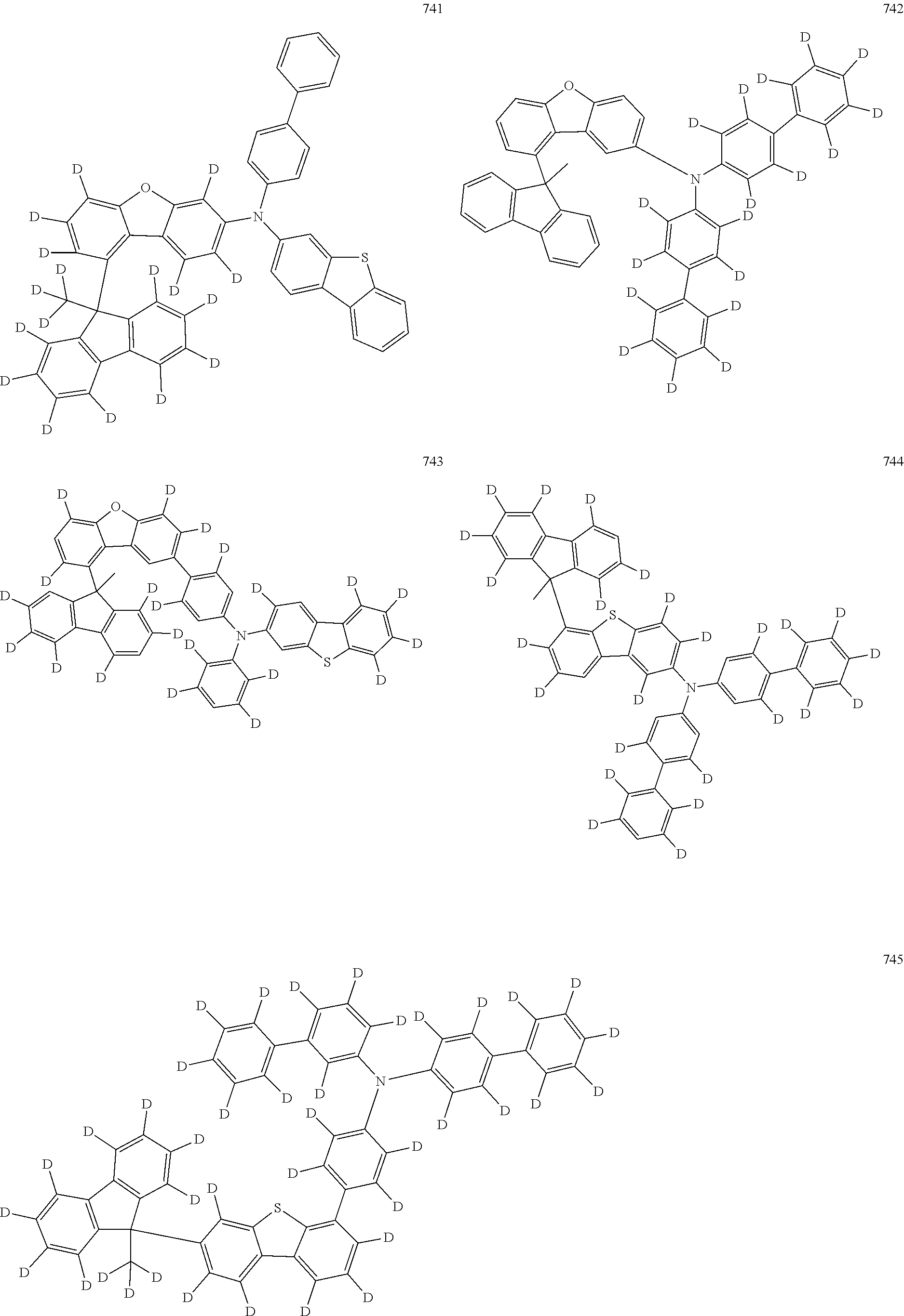

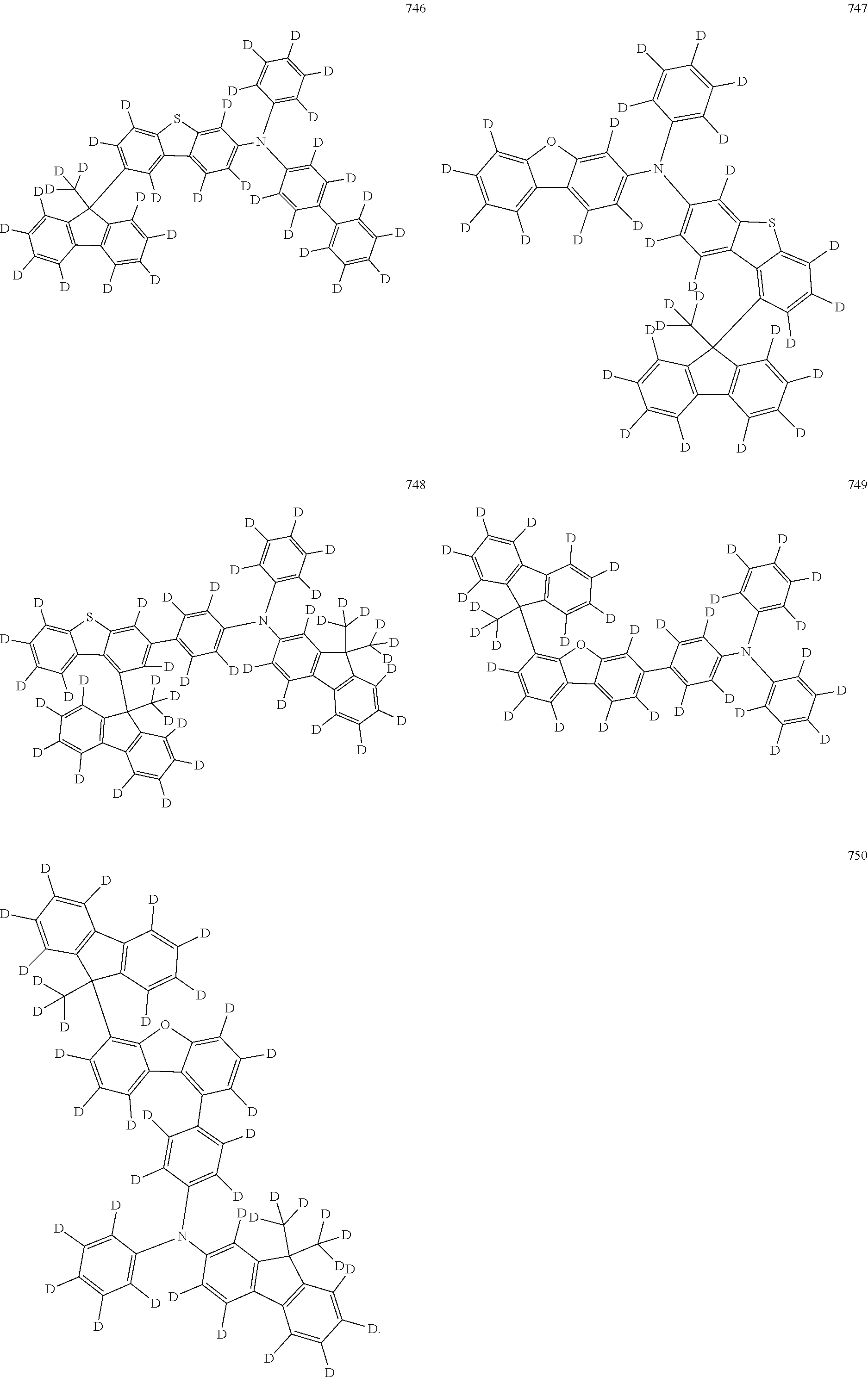

8. An organic light emitting device comprising a first electrode, a second electrode, and an organic material layer having one or more layers provided between the first electrode and the second electrode, wherein one or more layers of the organic material layer comprise the heterocyclic compound according to claim 1.

9. The organic light emitting device of claim 8, wherein the organic material layer comprises a hole transport layer, and the hole transport layer comprises the heterocyclic compound.

10. The organic light emitting device of claim 8, wherein the organic material layer comprises an electron blocking layer, and the electron blocking layer comprises the heterocyclic compound.

11. The organic light emitting device of claim 8, further comprising one or two or more layers selected from the group consisting of a light emitting layer, a hole injection layer, a hole transport layer, an electron blocking layer, an electron injection layer, an electron transport layer, a hole auxiliary layer, and a hole blocking layer.
